SEQUENCE LISTING (124 sequences)

SEQ ID NO: 1 - protein, 252 aa, Artificial Sequence
  Synthetic Construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 232 aa, Mus musculus
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 251 aa, Artificial Sequence
  Synthetic Construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 231 aa, Artificial Sequence
  Synthetic Construct
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 258 aa, Artificial Sequence
  Synthetic Construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 6 - protein, 238 aa, Artificial Sequence
  Synthetic Construct
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 7 - protein, 257 aa, Artificial Sequence
  Synthetic Construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 8 - protein, 237 aa, Artificial Sequence
  Synthetic Construct
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 9 - protein, 262 aa, Artificial Sequence
  Synthetic Construct
MYRMQLLSCIALSLALVTNSMVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 10 - protein, 242 aa, Artificial Sequence
  Synthetic Construct
MVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 11 - protein, 236 aa, Artificial Sequence
  Synthetic Construct
MVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 258 aa, Artificial Sequence
  Synthetic Construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 13 - protein, 238 aa, Artificial Sequence
  Synthetic Construct
PRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 14 - protein, 257 aa, Artificial Sequence
  Synthetic Construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 15 - protein, 237 aa, Artificial Sequence
  Synthetic Construct
MVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 16 - protein, 252 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 18 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 19 - protein, 252 aa, Artificial Sequence
  Synthetic Construct
MKWVTFISLLFLFSSAYSISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 234 aa, Artificial Sequence
  Synthetic Construct
ISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 266 aa, Artificial Sequence
  Synthetic Construct
MKWVTFISLLFLFSSAYSISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 22 - protein, 248 aa, Artificial Sequence
  Synthetic Construct
ISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 23 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 24 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 25 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGGGGGSEQKLISEEDL

SEQ ID NO: 26 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGGGGGSEQKLISEEDL

SEQ ID NO: 27 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 28 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 29 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 30 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 31 - protein, 259 aa, Artificial Sequence
  Synthetic Construct
MKWVTFISLLFLFSSAYSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 32 - protein, 245 aa, Artificial Sequence
  Synthetic Construct
MKWVTFISLLFLFSSAYSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 231 aa, Artificial Sequence
  Synthetic Construct
MVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 250 aa, Artificial Sequence
  Synthetic Construct
MKWVTFISLLFLFSSAYSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 245 aa, Artificial Sequence
  Synthetic Construct
MGWSCIILFLVATATGVHSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 226 aa, Artificial Sequence
  Synthetic Construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 226 aa, Artificial Sequence
  Synthetic Construct
KTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 38 - protein, 259 aa, Artificial Sequence
  Synthetic Construct
MKWVTFISLLFLFSSAYSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 39 - protein, 226 aa, Artificial Sequence
  Synthetic Construct
  MISC_FEATURE (32)..(32): Xaa is Met or Trp
  MISC_FEATURE (34)..(34): Xaa is Ser or Thr
  MISC_FEATURE (36)..(36): Xaa is Thr or Glu
  MISC_FEATURE (136)..(136): Xaa is Asp or Glu
  MISC_FEATURE (138)..(138): Xaa is Leu or Met
  MISC_FEATURE (208)..(208): Xaa is Met or Leu
  MISC_FEATURE (214)..(214): Xaa is Asn or Ser
DKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 40 - protein, 226 aa, Homo sapiens
  MISC_FEATURE (136)..(136): Xaa is Asp or Glu
  MISC_FEATURE (138)..(138): Xaa is Leu or Met
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 226 aa, Artificial Sequence
  Synthetic Construct
  MISC_FEATURE (136)..(136): Xaa is Asp or Glu
  MISC_FEATURE (138)..(138): Xaa is Leu or Met
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 226 aa, Artificial Sequence
  Synthetic Construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 226 aa, Artificial Sequence
  Synthetic Construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 226 aa, Artificial Sequence
  Synthetic Construct
  MISC_FEATURE (136)..(136): Xaa is Asp or Glu
  MISC_FEATURE (138)..(138): Xaa is Leu or Met
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 45 - protein, 226 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 46 - protein, 226 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 47 - protein, 265 aa, Artificial Sequence
  Synthetic Construct
  MISC_FEATURE (71)..(71): Xaa is Met or Trp
  MISC_FEATURE (73)..(73): Xaa is Ser or Thr
  MISC_FEATURE (75)..(75): Xaa is Thr or Glu
  MISC_FEATURE (175)..(175): Xaa is Asp or Glu
  MISC_FEATURE (177)..(177): Xaa is Leu or Met
  MISC_FEATURE (247)..(247): Xaa is Met or Leu
  MISC_FEATURE (253)..(253): Xaa is Asn or Ser
MGWSCIILFLVATATGVHSNVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 48 - protein, 266 aa, Artificial Sequence
  Synthetic Construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 266 aa, Artificial Sequence
  Synthetic Construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 266 aa, Artificial Sequence
  Synthetic Construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 51 - protein, 266 aa, Artificial Sequence
  Synthetic Construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 52 - protein, 266 aa, Artificial Sequence
  Synthetic Construct
MGWSCIILFLVATATGVHSNVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 266 aa, Artificial Sequence
  Synthetic Construct
MGWSCIILFLVATATGVHSNVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 267 aa, Artificial Sequence
  Synthetic Construct
MGWSCIILFLVATATGVHSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSEQKLISEEDL

SEQ ID NO: 55 - protein, 267 aa, Artificial Sequence
  Synthetic Construct
MGWSCIILFLVATATGVHSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSEQKLISEEDL

SEQ ID NO: 56 - protein, 267 aa, Artificial Sequence
  Synthetic Construct
MGWSCIILFLVATATGVHSISAMVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 57 - protein, 267 aa, Artificial Sequence
  Synthetic Construct
MGWSCIILFLVATATGVHSISAMVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 58 - protein, 275 aa, Artificial Sequence
  Synthetic Construct
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSQRNPRLRLIRRHPTLRIPPI

SEQ ID NO: 59 - protein, 275 aa, Artificial Sequence
  Synthetic Construct
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSQRNPRLRLIRRHPTLRIPPI

SEQ ID NO: 60 - protein, 247 aa, Artificial Sequence
  Synthetic Construct
  MISC_FEATURE (20)..(20): Xaa is Cys or Ser
  MISC_FEATURE (52)..(52): Xaa is Met or Trp
  MISC_FEATURE (54)..(54): Xaa is Ser or Thr
  MISC_FEATURE (56)..(56): Xaa is Thr or Glu
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 61 - protein, 247 aa, Artificial Sequence
  Synthetic Construct
  MISC_FEATURE (52)..(52): Xaa is Met or Trp
  MISC_FEATURE (54)..(54): Xaa is Ser or Thr
  MISC_FEATURE (56)..(56): Xaa is Thr or Glu
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 62 - protein, 247 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 66 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 67 - protein, 247 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 247 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 246 aa, Artificial Sequence
  Synthetic Construct
  MISC_FEATURE (20)..(20): Xaa is Cys or Ser
  MISC_FEATURE (52)..(52): Xaa is Met or Trp
  MISC_FEATURE (54)..(54): Xaa is Ser or Thr
  MISC_FEATURE (56)..(56): Xaa is Thr or Glu
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 70 - protein, 246 aa, Artificial Sequence
  Synthetic Construct
  MISC_FEATURE (52)..(52): Xaa is Met or Trp
  MISC_FEATURE (54)..(54): Xaa is Ser or Thr
  MISC_FEATURE (56)..(56): Xaa is Thr or Glu
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 71 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 75 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 76 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 246 aa, Artificial Sequence
  Synthetic Construct
  MISC_FEATURE (19)..(19): Xaa is Cys or Ser
  MISC_FEATURE (51)..(51): Xaa is Met or Trp
  MISC_FEATURE (53)..(53): Xaa is Ser or Thr
  MISC_FEATURE (55)..(55): Xaa is Thr or Glu
  MISC_FEATURE (155)..(155): Xaa is Asp or Glu
  MISC_FEATURE (157)..(157): Xaa is Leu or Met
  MISC_FEATURE (227)..(227): Xaa is Met or Leu
  MISC_FEATURE (233)..(233): Xaa is Asn or Ser
VNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 79 - protein, 246 aa, Artificial Sequence
  Synthetic Construct
  MISC_FEATURE (51)..(51): Xaa is Met or Trp
  MISC_FEATURE (53)..(53): Xaa is Ser or Thr
  MISC_FEATURE (55)..(55): Xaa is Thr or Glu
  MISC_FEATURE (155)..(155): Xaa is Asp or Glu
  MISC_FEATURE (157)..(157): Xaa is Leu or Met
  MISC_FEATURE (227)..(227): Xaa is Met or Leu
  MISC_FEATURE (233)..(233): Xaa is Asn or Ser
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 80 - protein, 246 aa, Artificial Sequence
  Synthetic Construct
  MISC_FEATURE (155)..(155): Xaa is Asp or Glu
  MISC_FEATURE (157)..(157): Xaa is Leu or Met
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 246 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 246 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 246 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 84 - protein, 246 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 85 - protein, 246 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 246 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 245 aa, Artificial Sequence
  Synthetic Construct
  MISC_FEATURE (19)..(19): Xaa is Cys or Ser
  MISC_FEATURE (51)..(51): Xaa is Met or Trp
  MISC_FEATURE (53)..(53): Xaa is Ser or Thr
  MISC_FEATURE (55)..(55): Xaa is Thr or Glu
  MISC_FEATURE (155)..(155): Xaa is Asp or Glu
  MISC_FEATURE (157)..(157): Xaa is Leu or Met
  MISC_FEATURE (227)..(227): Xaa is Met or Leu
  MISC_FEATURE (233)..(233): Xaa is Asn or Ser
VNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 88 - protein, 245 aa, Artificial Sequence
  Synthetic Construct
  MISC_FEATURE (51)..(51): Xaa is Met or Trp
  MISC_FEATURE (53)..(53): Xaa is Ser or Thr
  MISC_FEATURE (55)..(55): Xaa is Thr or Glu
  MISC_FEATURE (155)..(155): Xaa is Asp or Glu
  MISC_FEATURE (157)..(157): Xaa is Leu or Met
  MISC_FEATURE (227)..(227): Xaa is Met or Leu
  MISC_FEATURE (233)..(233): Xaa is Asn or Ser
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 89 - protein, 245 aa, Artificial Sequence
  Synthetic Construct
  MISC_FEATURE (155)..(155): Xaa is Asp or Glu
  MISC_FEATURE (157)..(157): Xaa is Leu or Met
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 245 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 245 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 245 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 93 - protein, 245 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 94 - protein, 245 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 245 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 585 aa, Homo sapiens [sequence omitted]

SEQ ID NO: 97 - protein, 591 aa, Homo sapiens
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 98 - protein, 590 aa, Mus musculus
RGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 99 - protein, 6 aa, Artificial Sequence
  Synthetic Construct
HHHHHH

SEQ ID NO: 100 - protein, 20 aa, Artificial Sequence
  Synthetic Construct
MYRMQLLSCIALSLALVTNS

SEQ ID NO: 101 - protein, 22 aa, Artificial Sequence
  Synthetic Construct
  MISC_FEATURE (1)..(2): Xaa is any amino acid
  MISC_FEATURE (4)..(8): Xaa is any amino acid
  MISC_FEATURE (10)..(11): Xaa is any amino acid
  MISC_FEATURE (14)..(14): Xaa is any amino acid
  MISC_FEATURE (18)..(20): Xaa is any amino acid
XXCXXXXXCXXFCXDWPXXXSC

SEQ ID NO: 102 - protein, 9 aa, Artificial Sequence
  Synthetic Construct
  MISC_FEATURE (4)..(6): Xaa is any amino acid
VCYXXXICF

SEQ ID NO: 103 - protein, 7 aa, Artificial Sequence
  Synthetic Construct
  MISC_FEATURE (3)..(3): Xaa is any amino aicd
  MISC_FEATURE (6)..(6): Xaa is any amino aicd
CYXPGXC

SEQ ID NO: 104 - protein, 11 aa, Artificial Sequence
  Synthetic Construct
  MISC_FEATURE (2)..(2): Xaa is any amino acid
  MISC_FEATURE (6)..(6): Xaa is any amino acid
DXCLPXWGCLW

SEQ ID NO: 105 - protein, 12 aa, Artificial Sequence
  Synthetic Construct
  MISC_FEATURE (4)..(5): Xaa is any amino acid
  MISC_FEATURE (7)..(7): Xaa is any amino acid
  MISC_FEATURE (9)..(9): Xaa is any amino acid
WCDXXLXAXDLC

SEQ ID NO: 106 - protein, 10 aa, Artificial Sequence
  Synthetic Construct
  MISC_FEATURE (4)..(4): Xaa is any amino acid
DLVXLGLECW

SEQ ID NO: 107 - protein, 11 aa, Artificial Sequence
  Synthetic Construct
DLCLRDWGCLW

SEQ ID NO: 108 - protein, 11 aa, Artificial Sequence
  Synthetic Construct
DICLPRWGCLW

SEQ ID NO: 109 - protein, 15 aa, Artificial Sequence
  Synthetic Construct
MEDICLPRWGCLWGD

SEQ ID NO: 110 - protein, 20 aa, Artificial Sequence
  Synthetic Construct
QRLMEDICLPRWGCLWEDDE

SEQ ID NO: 111 - protein, 20 aa, Artificial Sequence
  Synthetic Construct
QGLIGDICLPRWGCLWGRSV

SEQ ID NO: 112 - protein, 21 aa, Artificial Sequence
  Synthetic Construct
QGLIGDICLPRWGCLWGRSVK

SEQ ID NO: 113 - protein, 15 aa, Artificial Sequence
  Synthetic Construct
EDICLPRWGCLWEDD

SEQ ID NO: 114 - protein, 18 aa, Artificial Sequence
  Synthetic Construct
RLMEDICLPRWGCLWEDD

SEQ ID NO: 115 - protein, 16 aa, Artificial Sequence
  Synthetic Construct
MEDICLPRWGCLWEDD

SEQ ID NO: 116 - protein, 15 aa, Artificial Sequence
  Synthetic Construct
MEDICLPRWGCLWED

SEQ ID NO: 117 - protein, 18 aa, Artificial Sequence
  Synthetic Construct
RLMEDICLARWGCLWEDD

SEQ ID NO: 118 - protein, 20 aa, Artificial Sequence
  Synthetic Construct
EVRSFCTRWPAEKSCKPLRG

SEQ ID NO: 119 - protein, 20 aa, Artificial Sequence
  Synthetic Construct
RAPESFVCYWETICFERSEQ

SEQ ID NO: 120 - protein, 11 aa, Artificial Sequence
  Synthetic Construct
EMCYFPGICWM

SEQ ID NO: 121 - protein, 330 aa, Homo sapiens [sequence omitted]

SEQ ID NO: 122 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 123 - protein, 378 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 124 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2023/0082611 A1
(43) Pub. Date: Mar. 16, 2023

(54) COMPOSITIONS AND METHODS FOR THE TREATMENT OF RESPIRATORY SYNCYTIAL VIRUS

(71) Applicant: Cidara Therapeutics, Inc., San Diego, CA (US)

(72) Inventors: Allen BORCHARDT, San Diego, CA (US); Thomas P. BRADY, San Diego, CA (US); Zhi-Yong CHEN, La Jolla, CA (US); Quyen-Quyen Thuy DO, San Diego, CA (US); Travis James HAUSSENER, San Diego, CA (US); Alain NONCOVICH, La Jolla, CA (US); Leslie W. TARI, Solana Beach, CA (US)

(21) Appl. No.: 17/618,347

(22) PCT Filed: Jun. 12, 2020
(86) PCT No.: PCT/US2020/037606

§ 371 Date: Dec. 10, 2021

Related U.S. Application Data
(60) Provisional application No. 62/994,203, filed on Mar. 24, 2020.
(60) Provisional application No. 62/970,506, filed on Feb. 5, 2020.
(60) Provisional application No. 62/959,772, filed on Jan. 10, 2020.
(60) Provisional application No. 62/897,964, filed on Sep. 9, 2019.
(60) Provisional application No. 62/861,127, filed on Jun. 13, 2019.

(51) Int. Cl. A61K 47/68; A61K 45/06; A61K 47/54

(52) U.S. Cl. CPC A61K 47/6811; A61K 45/06; A61K 47/545

(57) ABSTRACT

Compositions and methods for the treatment of viral infections include conjugates containing inhibitors of viral RSV F protein (e.g., Presatovir, MDT 637, JNJ 179, or an analog thereof) linked to an Fc monomer, an Fc domain, and Fc-binding peptide, an albumin protein, or albumin-binding peptide. In particular, conjugates can be used in the treatment of viral infections (e.g., RSV infections).

### BACKGROUND

The need for novel antiviral treatments for respiratory syncytial virus (RSV) is significant and especially critical in the medical field. RSV is the causative agent of respiratory tract infection in several vulnerable patient populations, including children up to the age of two, immunocompromised patients, and the elderly. The viral infection progresses from the upper to the lower respiratory tract, and this results in airway inflammation, bronchiolitis, pneumonia and, in extreme cases, respiratory failure. In the aged community in the U.S., it was found that over four years RSV resulted in 177,500 hospital admissions and 14,000 deaths. RSV is a common infection in infants, almost all of whom will be infected in the first 24 months of life. Severe disease resulting from RSV infection is estimated to be the cause of approximately 3.2 million hospitalizations worldwide and around 66,000 deaths in children less than 5 years old.

The development of antiviral treatments for RSV has been a continuing challenge. Palivizumab, a monoclonal antibody, is approved for prophylactic use but is only 60% effective at reducing hospitalization rates. Ribavirin is approved as an inhaled treatment option in infants but has very limited efficacy and significant safety concerns for caregivers. The standard of care for RSV-infected patients is supportive, including fluids and oxygen. New, more effective therapies for treating RSV are needed.

### SUMMARY

The disclosure relates to conjugates, compositions, and methods for inhibiting viral growth, and methods for the treatment of viral infections. In particular, such conjugates contain monomers or dimers of a moiety that inhibits respiratory syncytial virus (RSV) fusion protein (F protein) (e.g., Presatovir, MDT 637, JNJ 179, or an analog thereof) conjugated to Fc monomers, Fc domains, Fc-binding peptides, albumin proteins, or albumin protein-binding peptides. The RSV F protein inhibitor (e.g., Presatovir, MDT 637, JNJ 179, or an analog thereof) in the conjugates targets RSV fusion protein on the surface of the viral particle. The Fc monomers or Fc domains in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC), thus leading to the engulfment and destruction of viral particles by immune cells and further enhancing the antiviral activity of the conjugates. The albumin or albumin-binding peptide may extend the half-life of the conjugate, for example, by binding of albumin to the recycling neonatal Fc receptor. Such compositions are useful in methods for the inhibition of viral growth and in methods for the treatment of viral infections, such as those caused by an RSV A and RSV B.

In a first aspect, the invention features a conjugate described by any one of formulas (D-I), (M-I), (1), or (2):

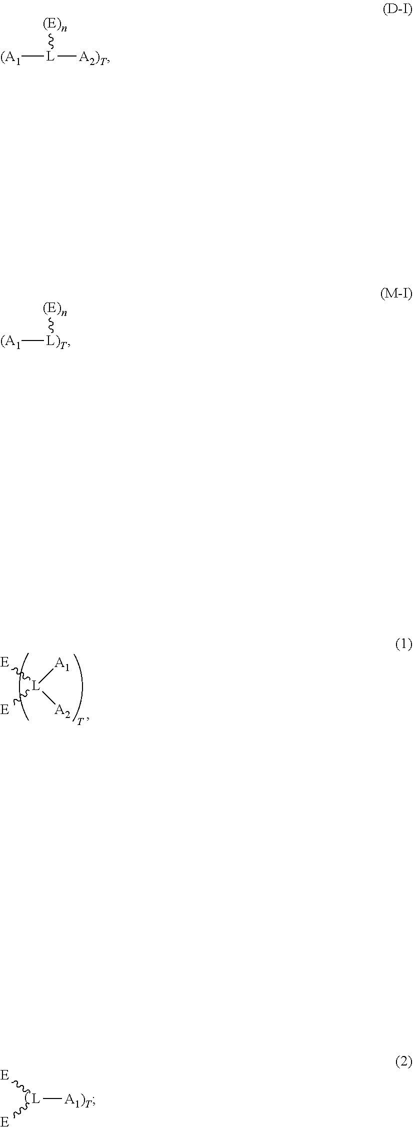

wherein each A₁ and each A₂ is independently selected from any one of formulas (A-I)-(A-III):

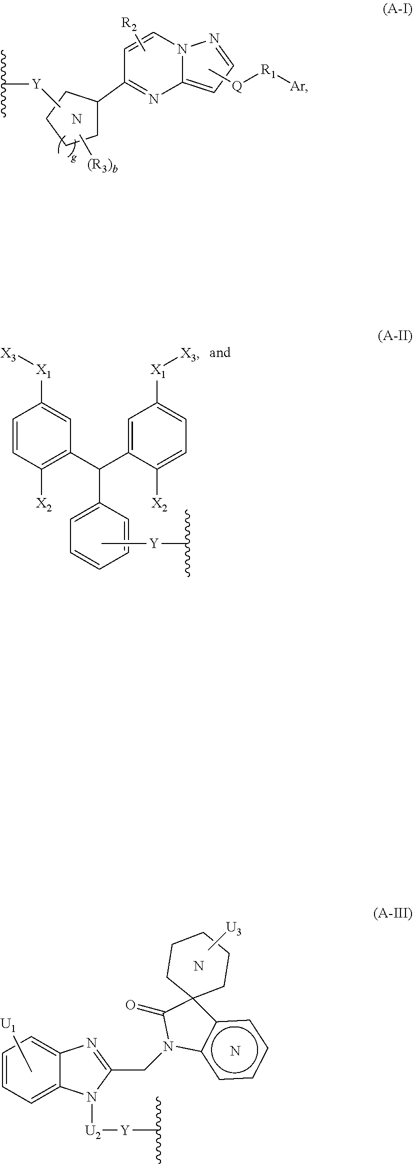

Q is selected from optionally substituted C₁-C₂₀ alkyl, optionally substituted C₃-C₂₀ cycloalkyl, optionally substituted C₂-C₂₀ heterocycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;

R₁, each X₁, and Y are each independently selected from —O—, —S—, —NR₅—, —CH═N—, —C(C═O)O—, —(C═O)NH—, —(C═O)—, —O(C═O)NR₅—, —O(C═S)NR₅—, —O(C═O)O—, —O(C═O)—, —NH(C═O)O—, —NH(C═O)—, —NH(C═NH)—, —NH(C═O)NR₅—, —NH(C═NH)NR₅—, —NH(C═S)NR₅—, —NH(C═S)—, —OCH₂(C═O)NR₅—, —R₅OR₆C(═O)NH—, —R₅NH(C═O)—, —R₅N—, —NH(SO₂)—, —NH(SO₂)NR₅—, —OR₆—, —NHR₆—, —SO₂—, and —SR₆—;

R₂, each R₃, each X₂, and U₁, are each independently selected from OH, halogen, nitrile, nitro, optionally substituted amine, optionally substituted imine, optionally substituted C₁-C₂₀ alkamino, optionally substituted sulfhydryl, optionally substituted carboxyl, optionally substituted cyano, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;

each X₃ is independently selected from optionally substituted C₁-C₂₀ alkyl, optionally substituted C₃-C₂₀ cycloalkyl, optionally substituted C₂-C₂₀ heterocycloalkyl; optionally substituted C₅-C₁₅ aryl, and optionally substituted C₁-C₁₅ heteroaryl;

U₂ is a substituent of the ring nitrogen atom and is selected from optionally substituted C₁-C₂₀ alkyl, optionally substituted C₃-C₂₀ cycloalkyl, optionally substituted C₂-C₂₀ heterocycloalkyl, optionally substituted C₅-C₁₅ aryl, optionally substituted C₃-C₁₅ heteroaryl, and a bond;

U₃ is a substituent of ring nitrogen atom and is selected from H, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy, optionally substituted C₁-C₂₀ alkamino, optionally substituted carboxyl, optionally substituted cyano;

Ar is selected from optionally substituted C₃-C₂₀ cycloalkyl, optionally substituted C₂-C₂₀ heterocycloalkyl, optionally substituted C₅-C₁₅ aryl, and optionally substituted C₁-C₁₅ heteroaryl;

R₅ and R₆ are each independently selected from H, optionally substituted C₁-C₂₀ alkyl, optionally substituted C₃-C₂₀ cycloalkyl, optionally substituted C₂-C₂₀ heterocycloalkyl; optionally substituted C₅-C₁₅ aryl, and optionally substituted C₂-C₁₅ heteroaryl;

b and g are each independently 0, 1, 2, or 3;

n is 1 or 2;

when n is 1, each E includes an Fc domain monomer (e.g., an Fc domain monomer having the sequence of any one of SEQ ID NOs: 1-95), an albumin protein (e.g., an albumin protein having the sequence of any one of SEQ ID NOs: 96-98), an albumin protein-binding peptide, or an Fc-binding peptide;

when n is 2, each E includes an Fc domain monomer (e.g., an Fc domain monomer having the sequence of any one of SEQ ID NOs: 1-95), wherein the Fc domain monomers dimerize to form and Fc domain;

L is a linker covalently attached to E and to each Y of each of A₁ or A₁ and A₂; and

T is an integer from 1 to 20 (e.g., 1, 2, 3, 4, 5, 6, 7, 8, 9, 10, 11, 12, 13, 14, 15, 16, 17, 18, 19, or 20), and each squiggly line in formulas (D-I), (M-I), (1), or (2) indicates that L is covalently attached (e.g., by way of a covalent bond or linker) to each E; or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁-L or each A₁-L-A₂ may be independently selected (e.g., independently selected from any of the A₁-L or A₁-L-A₂ structures described herein).

In preferred embodiments of any of the aspects described herein, n is 2 and each E includes an Fc domain monomer (e.g., an Fc domain monomer having the sequence of any one of SEQ ID NOs: 1-95). In a conjugate having two Fc domain monomers (e.g., a conjugate of formula (1), formula (2), formula (D-I) where n equals 2, or (M-I) where n equals 2), the Fc domain monomers dimerize to form an Fc domain.

In another aspect, the invention features a conjugate described by formula (D-I):

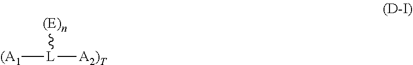

wherein each E includes an Fc domain monomer (e.g., an Fc domain monomer having the sequence of any one of SEQ ID NOs: 1-95); L in each A₁-L-A₂ is a linker covalently attached (e.g., by way of a covalent bond or linker) to a sulfur atom of a hinge cysteine in E and to each of A₁ and A₂; n is 1 or 2 (e.g., when n is 2, the two Fc domain monomers dimerize to form and Fc domain); T is an integer from 1 to 20 (e.g., 1, 2, 3, 4, 5, 6, 7, 8, 9, 10, 11, 12, 13, 14, 15, 16, 17, 18, 19, or 20), and the squiggly line connected to the E indicates that each A₁-L-A₂ is covalently attached to a sulfur atom of a hinge cysteine in E, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁-L-A₂ may be independently selected (e.g., independently selected from any of the A₁-L-A₂ structures described herein).

In another aspect, the invention features a conjugate described by formula (D-I):

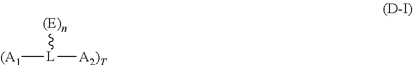

wherein each E includes an Fc domain monomer (e.g., an Fc domain monomer having the sequence of any one of SEQ ID NOs: 1-95); L in each A₁-L-A₂ is a linker covalently attached to a nitrogen atom of a surface exposed lysine in E and to each of A₁ and A₂; n is 1 or 2 (e.g., when n is 2, the two Fc domain monomers dimerize to form and Fc domain); T is an integer from 1 to 20 (e.g., 1, 2, 3, 4, 5, 6, 7, 8, 9, 10, 11, 12, 13, 14, 15, 16, 17, 18, 19, or 20), and the squiggly line connected to the E indicates that each A₁-L-A₂ is covalently attached (e.g., by way of a covalent bond or linker) to the nitrogen atom of a surface exposed lysine in E, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁-L-A₂ may be independently selected (e.g., independently selected from any of the A₁-L-A₂ structures described herein).

In another aspect, the invention features a conjugate described by formula (M-I):

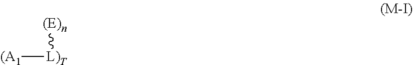

wherein each E includes an Fc domain monomer (e.g., an Fc domain monomer having the sequence of any one of SEQ ID NOs: 1-95); L in each L-A₁ is a linker covalently attached to a sulfur atom of a hinge cysteine in E and to A₁; n is 1 or 2 (e.g., when n is 2, the two Fc domain monomers dimerize to form and Fc domain); T is an integer from 1 to 20 (e.g., 1, 2, 3, 4, 5, 6, 7, 8, 9, 10, 11, 12, 13, 14, 15, 16, 17, 18, 19, or 20); and the squiggly line connected to E indicates that each L-A₁ is covalently attached to the sulfur atom of the hinge cysteine in E, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁ may be independently selected from a structure described by any one of formulas (A-I)-(A-III).

In another aspect, the invention features a conjugate described by formula (M-I):

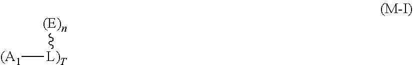

wherein each E includes an Fc domain monomer (e.g., an Fc domain monomer having the sequence of any one of SEQ ID NOs: 1-95); L in each L-A₁ is a linker covalently attached to a nitrogen atom of a surface exposed lysine in E and to A₁; n is 1 or 2 (e.g., when n is 2, the two Fc domain monomers dimerize to form and Fc domain); T is an integer from 1 to 20 (e.g., 1, 2, 3, 4, 5, 6, 7, 8, 9, 10, 11, 12, 13, 14, 15, 16, 17, 18, 19, or 20), the squiggly line connected to E indicates that each L-A₁ is covalently attached (e.g., by way of a covalent bond or linker) to the nitrogen atom of a surface exposed lysine in E, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁ may be independently selected from a structure described by any one of formulas (A-I)-(A-III).

In another aspect, the invention features a conjugate described by formula (1):

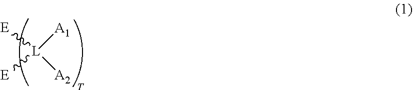

wherein each E includes an Fc domain monomer (e.g., an Fc domain monomer having the sequence of any one of SEQ ID NOs: 1-95); L in each A₁-L-A₂ is a linker covalently attached to a sulfur atom of a hinge cysteine in each E and to each of A₁ and A₂; T is an integer from 1 to 20 (e.g., 1, 2, 3, 4, 5, 6, 7, 8, 9, 10, 11, 12, 13, 14, 15, 16, 17, 18, 19, or 20), and the two squiggly lines connected to the two Es indicate that each A₁-L-A₂ is covalently attached to a pair of sulfur atoms of two hinge cysteines in the two Es, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁-L-A₂ may be independently selected (e.g., independently selected from any of the A₁-L-A₂ structures described herein).

In another aspect, the invention features a conjugate described by formula (2):

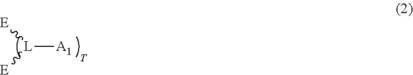

wherein each E includes an Fc domain monomer (e.g., an Fc domain monomer having the sequence of any one of SEQ ID NOs: 1-95); L in each L-A₁ is a linker covalently attached to a sulfur atom in a hinge cysteine in E and to A₁; T is an integer from 1 to 20 (e.g., 1, 2, 3, 4, 5, 6, 7, 8, 9, 10, 11, 12, 13, 14, 15, 16, 17, 18, 19, or 20), and the two squiggly lines connected to the two sulfur atoms indicate that each L-A₁ is covalently attached to a pair of sulfur atoms of two hinge cysteines in the two Es, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁ may be independently selected from a structure described by any one of formulas (A-I)-(A-III).

In some embodiments of any of the aspects described herein, each E includes an Fc domain monomer having the sequence of any one of SEQ ID NOs: 1-95.

In some embodiments, at least one of the pair of sulfur atoms is the sulfur atom corresponding to (e.g., the sulfur atom of) a hinge cysteine of SEQ ID NO: 10 or SEQ ID NO: 11, i.e., Cys10, Cys13, Cys16, or Cys18 of SEQ ID NO: 10 or SEQ ID NO: 11. In some embodiments, the pair of sulfur atoms are the sulfur atoms corresponding to (e.g., the sulfur atoms of) Cys10 and Cys13 in SEQ ID NO: 10 or SEQ ID NO: 11, Cys10 and Cys16 in SEQ ID NO: 10 or SEQ ID NO: 11, Cys 30 and Cys18 in SEQ ID NO: 10 or SEQ ID NO: 11, Cys13 and Cys 36 in SEQ ID NO: 10 or SEQ ID NO: 11, Cys13 and Cys 38 in SEQ ID NO: 10 or SEQ ID NO: 11, and/or Cys 36 and Cys 38 in SEQ ID NO: 10 or SEQ ID NO: 11.

In some embodiments, when T is 2, the pair of sulfur atoms are (e.g., the sulfur atoms corresponding to) Cys10 and Cys13 in SEQ ID NO: 10 or SEQ ID NO: 11 or Cys 36 and Cys 38 in SEQ ID NO: 10 or SEQ ID NO: 11.

In some embodiments, the pair of sulfur atoms include one sulfur atom of a cysteine from each E, i.e., L-A along with the sulfur atoms to which it is attached forms a bridge between two Fc domains (e.g., two Fc domains including the sequence of SEQ ID NO: 10 or SEQ ID NO: 11). In some embodiments, the pair of sulfur atoms are the sulfur atom corresponding to (e.g., the sulfur atom of) Cys10 of SEQ ID NO: 10 or SEQ ID NO: 11 from one E and the sulfur atom corresponding to (e.g., the sulfur atom of) Cys10 of SEQ ID NO: 10 or SEQ ID NO: 11 from another E. In some embodiments, the pair of sulfur atoms are the sulfur atom corresponding to (e.g., the sulfur atom of) Cys13 of SEQ ID NO: 10 or SEQ ID NO: 11 from one E and the sulfur atom corresponding to (e.g., the sulfur atom of) Cys13 of SEQ ID NO: 10 or SEQ ID NO: 11 from another E. In some embodiments, the pair of sulfur atoms are the sulfur atom corresponding to (e.g., the sulfur atom of) Cys16 of SEQ ID NO: 10 or SEQ ID NO: 11 from one E and the sulfur atom corresponding to (e.g., the sulfur atom of) Cys16 of SEQ ID NO: 10 or SEQ ID NO: 11 from another E. In some embodiments, the pair of sulfur atoms are the sulfur atom corresponding to (e.g., the sulfur atom of) Cys18 of SEQ ID NO: 10 or SEQ ID NO: 11 from one E and the sulfur atom corresponding to (e.g., the sulfur atom of) Cys18 of SEQ ID NO: 10 or SEQ ID NO: 11 from another E.

In some embodiments, when T is 2, the pairs of sulfur atoms are the sulfur atom corresponding to (e.g., the sulfur atom of) Cys10 of SEQ ID NO: 10 or SEQ ID NO: 11 from one E and the sulfur atom corresponding to (e.g., the sulfur atom of) Cys10 of SEQ ID NO: 10 or SEQ ID NO: 11 from another E and the sulfur atom corresponding to (e.g., the sulfur atom of) Cys13 of SEQ ID NO: 10 or SEQ ID NO: 11 from one E and the sulfur atom corresponding to (e.g., the sulfur atom of) Cys13 of SEQ ID NO: 10 or SEQ ID NO: 11 from another E. In some embodiments, when T is 2, the pairs of sulfur atoms are the sulfur atom corresponding to (e.g., the sulfur atom of) Cys10 of SEQ ID NO: 10 or SEQ ID NO: 11 from one E and the sulfur atom corresponding to (e.g., the sulfur atom of) Cys10 of SEQ ID NO: 10 or SEQ ID NO: 11 from another E and the sulfur atom corresponding to (e.g., the sulfur atom of) Cys16 of SEQ ID NO: 10 or SEQ ID NO: 11 from one E and the sulfur atom corresponding to (e.g., the sulfur atom of) Cys16 of SEQ ID NO: 10 or SEQ ID NO: 11 from another E. In some embodiments, when T is 2, the pairs of sulfur atoms are the sulfur atom corresponding to (e.g., the sulfur atom of) Cys10 of SEQ ID NO: 10 or SEQ ID NO: 11 from one E and the sulfur atom corresponding to (e.g., the sulfur atom of) Cys10 of SEQ ID NO: 10 or SEQ ID NO: 11 from another E and the sulfur atom corresponding to (e.g., the sulfur atom of) Cys18 of SEQ ID NO: 10 or SEQ ID NO: 11 from one E and the sulfur atom corresponding to (e.g., the sulfur atom of) Cys18 of SEQ ID NO: 10 or SEQ ID NO: 11 from another E.

In some embodiments, when T is 2, the pairs of sulfur atoms are the sulfur atom corresponding to (e.g., the sulfur atom of) Cys13 of SEQ ID NO: 10 or SEQ ID NO: 11 from one E and the sulfur atom corresponding to (e.g., the sulfur atom of) Cys13 of SEQ ID NO: 10 or SEQ ID NO: 11 from another E and the sulfur atom corresponding to (e.g., the sulfur atom of) Cys16 of SEQ ID NO: 10 or SEQ ID NO: 11 from one E and the sulfur atom corresponding to (e.g., the sulfur atom of) Cys16 of SEQ ID NO: 10 or SEQ ID NO: 11 from another E. In some embodiments, when T is 2, the pairs of sulfur atoms are the sulfur atom corresponding to (e.g., the sulfur atom of) Cys13 of SEQ ID NO: 10 or SEQ ID NO: 11 from one E and the sulfur atom corresponding to (e.g., the sulfur atom of) Cys13 of SEQ ID NO: 10 or SEQ ID NO: 11 from another E and the sulfur atom corresponding to (e.g., the sulfur atom of) Cys18 of SEQ ID NO: 10 or SEQ ID NO: 11 from one E and the sulfur atom corresponding to (e.g., the sulfur atom of) Cys18 of SEQ ID NO: 10 or SEQ ID NO: 11 from another E.

In some embodiments, when T is 2, the pairs of sulfur atoms are the sulfur atom corresponding to (e.g., the sulfur atom of) Cys16 of SEQ ID NO: 10 or SEQ ID NO: 11 from one E and the sulfur atom corresponding to (e.g., the sulfur atom of) Cys16 of SEQ ID NO: 10 or SEQ ID NO: 11 from another E and the sulfur atom corresponding to (e.g., the sulfur atom of) Cys18 of SEQ ID NO: 10 or SEQ ID NO: 11 from one E and the sulfur atom corresponding to (e.g., the sulfur atom of) Cys18 of SEQ ID NO: 10 or SEQ ID NO: 11 from another E.

In some embodiments, when T is 3, the pairs of sulfur atoms are the sulfur atom corresponding to (e.g., the sulfur atom of) Cys10 of SEQ ID NO: 10 or SEQ ID NO: 11 from one E and the sulfur atom corresponding to (e.g., the sulfur atom of) Cys10 of SEQ ID NO: 10 or SEQ ID NO: 11 from another E; the sulfur atom corresponding to (e.g., the sulfur atom of) Cys13 of SEQ ID NO: 10 or SEQ ID NO: 11 from one E and the sulfur atom corresponding to (e.g., the sulfur atom of) Cys13 of SEQ ID NO: 10 or SEQ ID NO: 11 from another E; and the sulfur atom corresponding to (e.g., the sulfur atom of) Cys16 of SEQ ID NO: 10 or SEQ ID NO: 11 from one E and the sulfur atom corresponding to (e.g., the sulfur atom of) Cys16 of SEQ ID NO: 10 or SEQ ID NO: 11 from another E. In some embodiments, when T is 3, the pairs of sulfur atoms are the sulfur atom corresponding to (e.g., the sulfur atom of) Cys10 of SEQ ID NO: 10 or SEQ ID NO: 11 from one E and the sulfur atom corresponding to (e.g., the sulfur atom of) Cys10 of SEQ ID NO: 10 or SEQ ID NO: 11 from another E; the sulfur atom corresponding to (e.g., the sulfur atom of) Cys13 of SEQ ID NO: 10 or SEQ ID NO: 11 from one E and the sulfur atom corresponding to (e.g., the sulfur atom of) Cys13 of SEQ ID NO: 10 or SEQ ID NO: 11 from another E; and the sulfur atom corresponding to (e.g., the sulfur atom of) Cys18 of SEQ ID NO: 10 or SEQ ID NO: 11 from one E and the sulfur atom corresponding to (e.g., the sulfur atom of) Cys18 of SEQ ID NO: 10 or SEQ ID NO: 11 from another E. In some embodiments, when T is 3, the pairs of sulfur atoms are the sulfur atom corresponding to (e.g., the sulfur atom of) Cys10 of SEQ ID NO: 10 or SEQ ID NO: 11 from one E and the sulfur atom corresponding to (e.g., the sulfur atom of) Cys10 of SEQ ID NO: 10 or SEQ ID NO: 11 from another E; the sulfur atom corresponding to (e.g., the sulfur atom of) Cys18 of SEQ ID NO: 10 or SEQ ID NO: 11 from one E and the sulfur atom corresponding to (e.g., the sulfur atom of) Cys18 of SEQ ID NO: 10 or SEQ ID NO: 11 from another E; and the sulfur atom corresponding to (e.g., the sulfur atom of) Cys16 of SEQ ID NO: 10 or SEQ ID NO: 11 from one E and the sulfur atom corresponding to (e.g., the sulfur atom of) Cys16 of SEQ ID NO: 10 or SEQ ID NO: 11 from another E. In some embodiments, when T is 3, the pairs of sulfur atoms are the sulfur atom corresponding to (e.g., the sulfur atom of) Cys13 of SEQ ID NO: 10 or SEQ ID NO: 11 from one E and the sulfur atom corresponding to (e.g., the sulfur atom of) Cys13 of SEQ ID NO: 10 or SEQ ID NO: 11 from another E; the sulfur atom corresponding to (e.g., the sulfur atom of) Cys18 of SEQ ID NO: 10 or SEQ ID NO: 11 from one E and the sulfur atom corresponding to (e.g., the sulfur atom of) Cys18 of SEQ ID NO: 10 or SEQ ID NO: 11 from another E; and the sulfur atom corresponding to (e.g., the sulfur atom of) Cys16 of SEQ ID NO: 10 or SEQ ID NO: 11 from one E and the sulfur atom corresponding to (e.g., the sulfur atom of) Cys16 of SEQ ID NO: 10 or SEQ ID NO: 11 from another E.

In some embodiments, when T is 3, the pairs of sulfur atoms are the sulfur atom corresponding to (e.g., the sulfur atom of) Cys10 of SEQ ID NO: 10 or SEQ ID NO: 11 from one E and the sulfur atom corresponding to (e.g., the sulfur atom of) Cys10 of SEQ ID NO: 10 or SEQ ID NO: 11 from another E; the sulfur atom corresponding to (e.g., the sulfur atom of) Cys13 of SEQ ID NO: 10 or SEQ ID NO: 11 from one E and the sulfur atom corresponding to (e.g., the sulfur atom of) Cys13 of SEQ ID NO: 10 or SEQ ID NO: 11 from another E; the sulfur atom corresponding to (e.g., the sulfur atom of) Cys16 of SEQ ID NO: 10 or SEQ ID NO: 11 from one E and the sulfur atom corresponding to (e.g., the sulfur atom of) Cys16 of SEQ ID NO: 10 or SEQ ID NO: 11 from another E; and the sulfur atom corresponding to (e.g., the sulfur atom of) Cys18 of SEQ ID NO: 10 or SEQ ID NO: 11 from one E and the sulfur atom corresponding to (e.g., the sulfur atom of) Cys18 of SEQ ID NO: 10 or SEQ ID NO: 11 from another E.

In some embodiments, the conjugate has the structure:

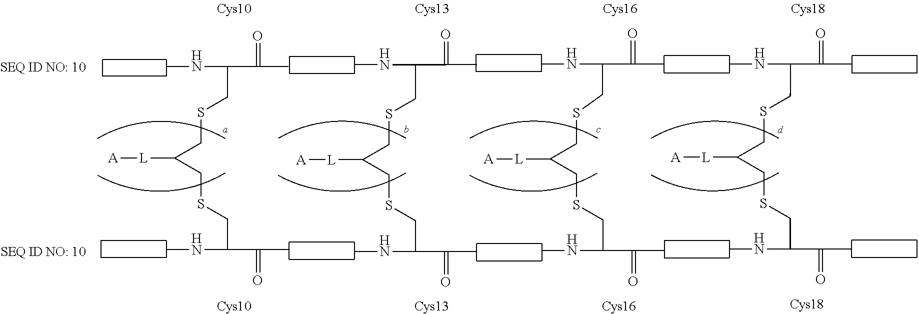

wherein each of a, b, c, and d is, independently, 0 or 1 and wherein when a, b, c, or d is 0, the two sulfur atoms form a disulfide bond.

In some embodiments, a is 1 and b, c, and d are 0. In some embodiments, a and b are 1 and c and d are 0. In some embodiments, a and c are 1 and b and d are 0. In some embodiments, a and d are 1 and b and c are 0. In some embodiments, a, b, and c are 1 and d is 0. In some embodiments, a, b, and d are 1 and c is 0. In some embodiments, a, c, and d are 1 and b is 0. In some embodiments, b and c are 1 and a and d are 0. In some embodiments, b and d are 1 and a and c are 0. In some embodiments, b, c, and d are 1 and a is 0. In some embodiments, c and d are 1 and a and b are 0. In some embodiments, a, b, c, and d are 1.

In some embodiments, at least one of the pair of sulfur atoms is the sulfur atom corresponding to (e.g., the sulfur atom of) a hinge cysteine of SEQ ID NO: 4 or SEQ ID NO: 33, i.e., Cys10 and/or Cys13. In some embodiments, the pair of sulfur atoms are the sulfur atoms corresponding to (e.g., the sulfur atoms of) Cys10 and Cys13 in SEQ ID NO: 4 or SEQ ID NO: 33.

In some embodiments, the pair of sulfur atoms include one sulfur atom of a cysteine from each E, i.e., L-A along with the sulfur atoms to which it is attached forms a bridge between two Fc domains (e.g., two Fc domains including the sequence of SEQ ID NO: 4 or SEQ ID NO: 33). In some embodiments, the pair of sulfur atoms are the sulfur atom corresponding to (e.g., the sulfur atom of) Cys10 of SEQ ID NO: 4 or SEQ ID NO: 33 from one E and the sulfur atom corresponding to (e.g., the sulfur atom of) Cys10 of SEQ ID NO: 4 or SEQ ID NO: 33 from another E. In some embodiments, the pair of sulfur atoms are the sulfur atom corresponding to (e.g., the sulfur atom of) Cys13 of SEQ ID NO: 4 or SEQ ID NO: 33 from one E and the sulfur atom corresponding to (e.g., the sulfur atom of) Cys13 of SEQ ID NO: 4 or SEQ ID NO: 33 from another E. In some embodiments, when T is 2, the pairs of sulfur atoms are the sulfur atom corresponding to (e.g., the sulfur atom of) Cys10 of SEQ ID NO: 4 or SEQ ID NO: 33 from one E and the sulfur atom corresponding to (e.g., the sulfur atom of) Cys10 of SEQ ID NO: 4 or SEQ ID NO: 33 from another E and the sulfur atom corresponding to (e.g., the sulfur atom of) Cys13 of SEQ ID NO: 4 or SEQ ID NO: 33 from one E and the sulfur atom corresponding to (e.g., the sulfur atom of) Cys13 of SEQ ID NO: 4 or SEQ ID NO: 33 from another E.

In some embodiments, the conjugate has the structure:

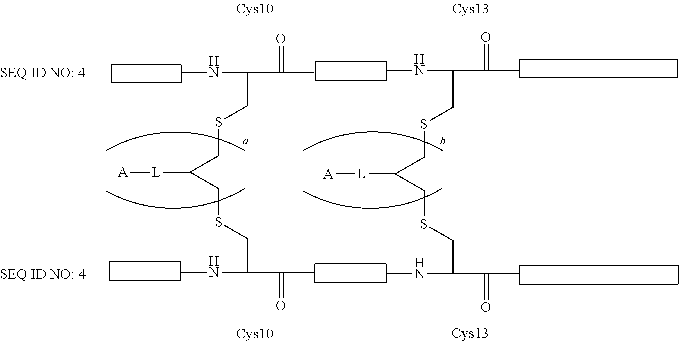

wherein each of a and b is, independently, 0 or 1 and wherein when a or b is 0, the two sulfur atoms form a disulfide bond. In some embodiments, a is 1 and b is 0. In some embodiments, a is 0 and b is 1. In some embodiments, a and b are 1.

In some embodiments, at least one of the pair of sulfur atoms is the sulfur atom corresponding to (e.g., the sulfur atom of) a hinge cysteine of SEQ ID NO: 8, i.e., Cys10 and/or Cys13. In some embodiments, the pair of sulfur atoms are the sulfur atoms corresponding to (e.g., the sulfur atoms of) Cys10 and Cys13 in SEQ ID NO: 8.

In some embodiments, the pair of sulfur atoms include one sulfur atom of a cysteine from each E, i.e., L-A along with the sulfur atoms to which it is attached forms a bridge between two Fc domains (e.g., two Fc domains including the sequence of SEQ ID NO: 8). In some embodiments, the pair of sulfur atoms are the sulfur atom corresponding to (e.g., the sulfur atom of) Cys10 of SEQ ID NO: 8 from one E and the sulfur atom corresponding to (e.g., the sulfur atom of) Cys10 of SEQ ID NO: 8 from another E. In some embodiments, the pair of sulfur atoms are the sulfur atom corresponding to (e.g., the sulfur atom of) Cys13 of SEQ ID NO: 8 from one E and the sulfur atom corresponding to (e.g., the sulfur atom of) Cys13 of SEQ ID NO: 8 from another E. In some embodiments, when T is 2, the pairs of sulfur atoms are the sulfur atom corresponding to (e.g., the sulfur atom of) Cys10 of SEQ ID NO: 8 from one E and the sulfur atom corresponding to (e.g., the sulfur atom of) Cys10 of SEQ ID NO: 8 from another E and the sulfur atom corresponding to (e.g., the sulfur atom of) Cys13 of SEQ ID NO: 8 from one E and the sulfur atom corresponding to (e.g., the sulfur atom of) Cys13 of SEQ ID NO: 8 from another E.

In some embodiments, the conjugate has the structure:

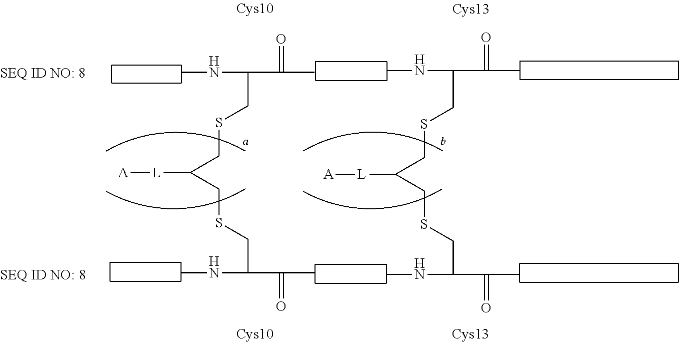

wherein each of a and b is, independently, 0 or 1 and wherein when a or b is 0, the two sulfur atoms form a disulfide bond. In some embodiments, a is 1 and b is 0. In some embodiments, a is 0 and b is 1. In some embodiments, a and b are 1.

In some embodiments, the conjugate has the structure:

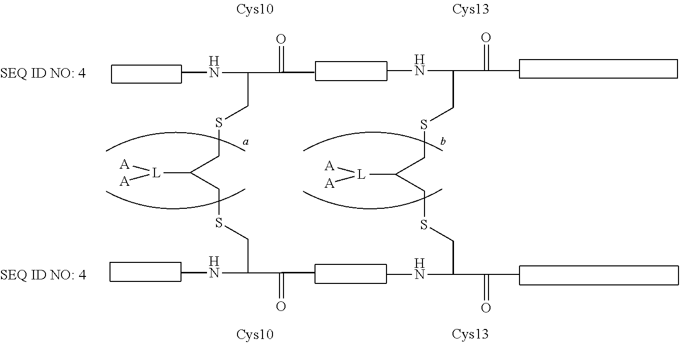

wherein each of a and b is, independently, 0 or 1 and wherein when a or b is 0, the two sulfur atoms form a disulfide bond. In some embodiments, a is 1 and b is 0. In some embodiments, a is 0 and b is 1. In some embodiments, a and b are 1.

In some embodiments, the conjugate has the structure:

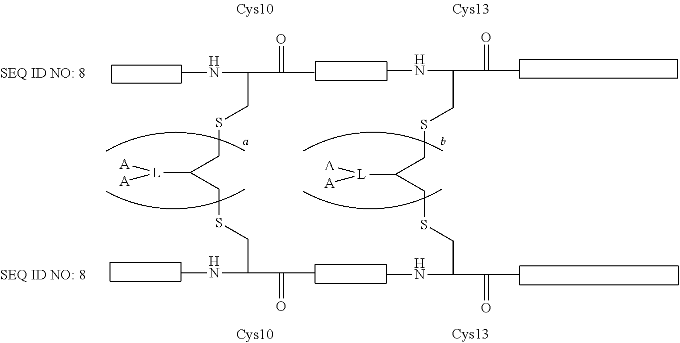

wherein each of a and b is, independently, 0 or 1 and wherein when a or b is 0, the two sulfur atoms form a disulfide bond. In some embodiments, a is 1 and b is 0. In some embodiments, a is 0 and b is 1. In some embodiments, a and b are 1.

In some embodiments, the conjugate has the structure:

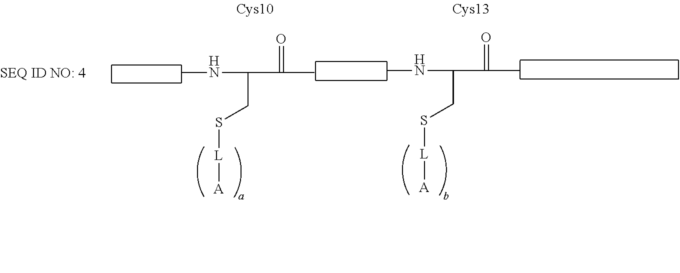

wherein each of a and b is, independently, 0 or 1 and wherein when a or b is 0, the sulfur atoms is a thiol. In some embodiments, a is 1 and b is 0. In some embodiments, a is 0 and b is 1. In some embodiments, a and b are 1.

In some embodiments of the previous three aspects, the nitrogen atom is the nitrogen of a surface exposed lysine, e.g., the nitrogen atom corresponding to (e.g., the nitrogen atom of) Lys35, Lys63, Lys77, Lys79, Lys106, Lys123, Lys129, Lys181, Lys203, Lys228, or Lys236 of SEQ ID NO: 10 or SEQ ID NO: 11. In some embodiments, the nitrogen atom is the nitrogen atom corresponding to (e.g., the nitrogen atom of) Lys65, Lys79, Lys108, Lys230, and/or Lys238 of SEQ ID NO: 10 or SEQ ID NO: 11.

In some embodiments, the conjugate has the structure:

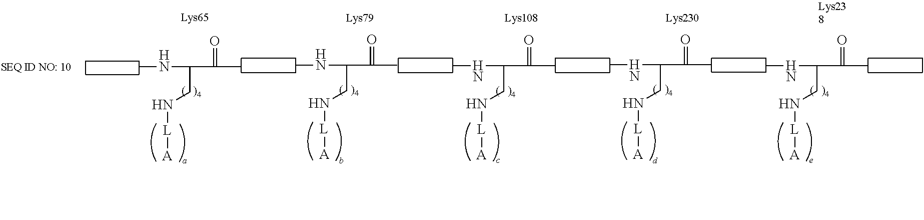

wherein each of a, b, c, d, and e is, independently, 0 or 1 and wherein when a, b, c, d, ore is 0, the two nitrogen atom is NH₂. In some embodiments, a is 1 and b, c, d, and e are 0. In some embodiments, b is 1 and a, c, d, and e are 0. In some embodiments, c is 1 and a, b, d, and e are 0. In some embodiments, d is 1 and a, b, c, and e are 0. In some embodiments, e is 1 and a, b, c, and d are 0. In some embodiments, a and b are 1 and c, d, and e are 0. In some embodiments, a and c are 1 and b, d, and e are 0. In some embodiments, a and d are 1 and b, c, and e are 0. In some embodiments, a and e are 1 and b, c, and d are 0. In some embodiments, b and c are 1 and a, d, and e are 0. In some embodiments, b and d are 1 and a, c, and e are 0. In some embodiments, b and e are 1 and a, c, and d are 0. In some embodiments, c and d are 1 and a, b, and e are 0. In some embodiments, c and e are 1 and a, b, and d are 0. In some embodiments, d and e are 1 and a, b, and c are 0. In some embodiments, a, b, and c are 1 and d and e are 0. In some embodiments, a, b, and d are 1 and c and e are 0. In some embodiments, a, b, and e are 1 and c and d are 0. In some embodiments, a, c, and d are 1 and b and e are 0. In some embodiments, a, c, and e are 1 and b and d are 0. In some embodiments, a, d, and e are 1 and b and c are 0. In some embodiments, b, c, and d are 1 and a and e are 0. In some embodiments, b, d, and e are 1 and a and c are 0. In some embodiments, c, d, and e are 1 and a and b are 0.

In some embodiments of the conjugates described herein, the conjugate forms a homodimer including an Fc domain. In some embodiments of any of the conjugates described herein, E homodimerizes with another E to form an Fc domain.

In another aspect, the invention features a conjugate described by formula (D-I):

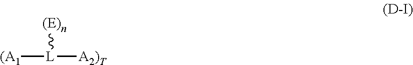

wherein E includes an albumin protein (e.g., an albumin protein having the sequence of any one of SEQ ID NOs: 96-98), an albumin protein-binding peptide, or an Fc-binding peptide; L in each A₁-L-A₂ is a linker independently covalently attached to a sulfur atom of a surface exposed cysteine or a nitrogen atom of a surface exposed lysine in E and to each of A₁ and A₂; n is 1; T is an integer from 1 to 20 (e.g., 1, 2, 3, 4, 5, 6, 7, 8, 9, 10, 11, 12, 13, 14, 15, 16, 17, 18, 19, or 20), and the squiggly line connected to the E indicates that each A₁-L-A₂ is independently covalently attached to the sulfur atom of a solvent-exposed cysteine or the nitrogen atom of a solvent-exposed lysine in E, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁-L-A₂ may be independently selected (e.g., independently selected from any of the A₁-L-A₂ structures described herein).

In another aspect, the invention features a conjugate described by formula (M-I):

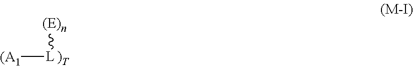

E includes an albumin protein (e.g., an albumin protein having the sequence of any one of SEQ ID NOs: 96-98), an albumin protein-binding peptide, or an Fc-binding peptide; L in each L-A₁ is a linker independently covalently attached to a sulfur atom of a surface exposed cysteine or a nitrogen atom of a surface exposed lysine in E and to A₁; n is 1; T is an integer from 1 to 20 (e.g., 1, 2, 3, 4, 5, 6, 7, 8, 9, 10, 11, 12, 13, 14, 15, 16, 17, 18, 19, or 20); and the squiggly line connected to E indicates that each L-A₁ is independently covalently attached to the sulfur atom of the solvent-exposed cysteine or the nitrogen atom of the solvent-exposed lysine in E, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁ may be independently selected from a structure described by any one of formulas (A-I)-(A-III).

In some embodiments, each E includes an albumin protein having the sequence of any one of SEQ ID NOs: 96-98.

In some embodiments, T is 1 and L-A is covalently attached to the sulfur atom corresponding to Cys34 of SEQ ID NO: 96.

In another aspect, the invention features an intermediate (Int) of Table 1. These intermediates include one or inhibitors of RSV F protein (e.g., Presatovir, MDT 637, JNJ 179, or an analog thereof) and a linker (e.g., a PEG₂-PEG₂₀ linker) and may be used in the synthesis of a conjugate described herein. Intermediates of Table 1 may be conjugated to, for example, an Fc domain or Fc domain monomer, albumin protein, albumin protein-binding peptide, or Fc-binding peptide (e.g., by way of a linker) by any suitable methods known to those of skill in the art, including any of the methods described or exemplified herein. In some embodiments, the conjugate (e.g., a conjugate described by any one of formulas (1), (2), (D-I)-(D-IV), or (M-I)-(M-IV)) includes E, wherein E is an Fc domain monomer or an Fc domain (e.g., an Fc domain monomer or an Fc domain, each Fc domain monomer having, independently, the sequence of any one of SEQ ID NOs: 1-95). In preferred embodiments, one or more nitrogen atoms of one or more surface exposed lysine residues of E or one or more sulfur atoms of one or more surface exposed cysteines in E is covalently conjugated to a linker (e.g., a PEG₂-PEG₂₀ linker). The linker conjugated to E may be functionalized such that it may react to form a covalent bond with any of the Ints described herein (e.g., an Int of Table 1). In preferred embodiments, E is conjugated to a linker functionalized with an azido group and the Int (e.g., an Int of Table 1) is functionalized with an alkyne group. Conjugation (e.g., by click chemistry) of the linker-azido of E and linker-alkyne of the Int forms a conjugate of the invention, for example a conjugate described by any one of formulas (1), (2), (D-I)-(D-IV), or (M-I)-(M-IV). In yet other embodiments, E is conjugated to a linker functionalized with an alkyne group and the Int (e.g., an Int of Table 1) is functionalized with an azido group. Conjugation (e.g., by click chemistry) of the linker-alkyne of E and the linker-azido of the Int forms a conjugate of the invention, for example a conjugate described by any one of formulas (1), (2), (D-I)-(D-IV), or (M-I)-(M-IV).

TABLE 1
Intermediates

| Inter- | |
|---|---|
| mediate | Structure |
| Int-1 | 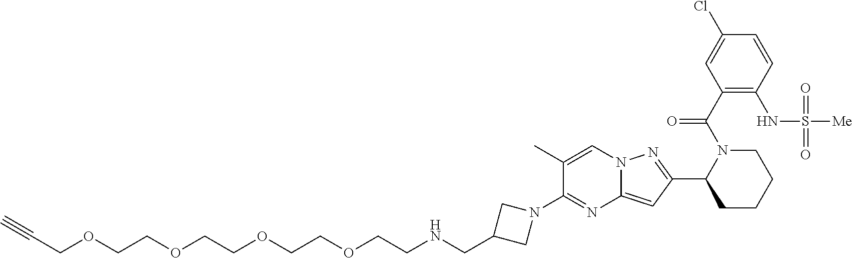 [SMILES: C#CCOCCOCCOCCOCCNCC1CN(c2nc3cc([C@@H]4CCCCN4C(=O)c4cc(Cl)ccc4NS(C)(=O)=O)nn3cc2C)C1] |
| Int-2 | 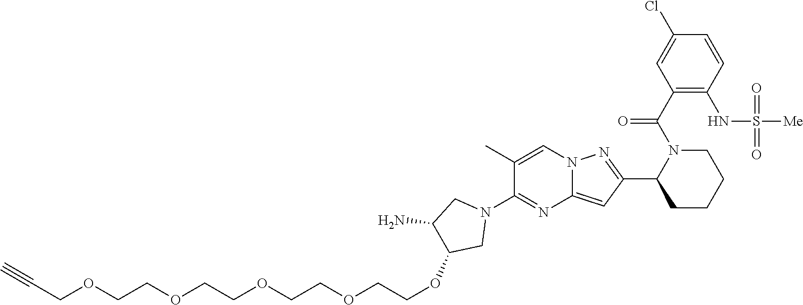 [SMILES: C#CCOCCOCCOCCOCCO[C@H]1CN(c2nc3cc([C@@H]4CCCCN4C(=O)c4cc(Cl)ccc4NS(C)(=O)=O)nn3cc2C)C[C@H]1N] |
| Int-3 | 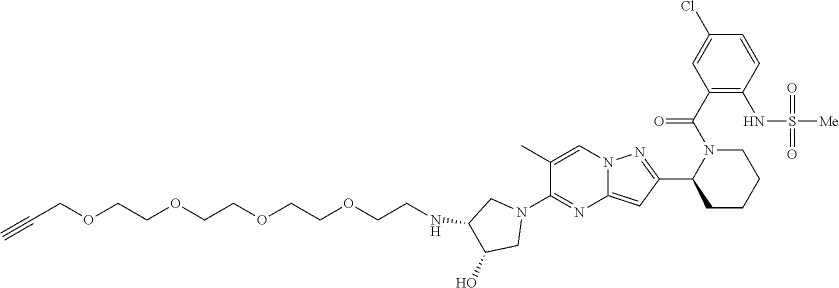 [SMILES: C#CCOCCOCCOCCOCCN[C@@H]1CN(c2nc3cc([C@@H]4CCCCN4C(=O)c4cc(Cl)ccc4NS(C)(=O)=O)nn3cc2C)C[C@@H]1O] |
| Int-4 | 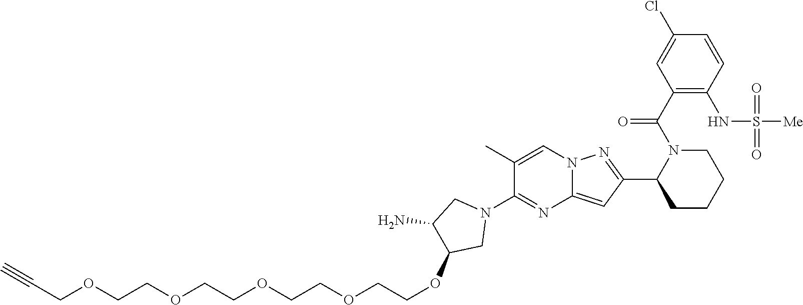 [SMILES: C#CCOCCOCCOCCOCCO[C@@H]1CN(c2nc3cc([C@@H]4CCCCN4C(=O)c4cc(Cl)ccc4NS(C)(=O)=O)nn3cc2C)C[C@H]1N] |
| Int-5 | 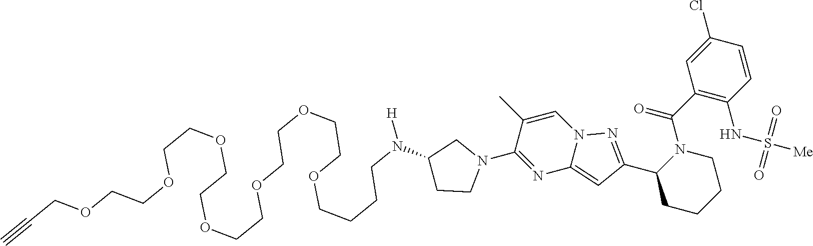 [SMILES: C#CCOCCOCCOCCOCCOCCOCCOCCCCN[C@H]1CCN(c2nc3cc([C@@H]4CCCCN4C(=O)c4cc(Cl)ccc4NS(C)(=O)=O)nn3cc2C)C1] |
| Int-6 | 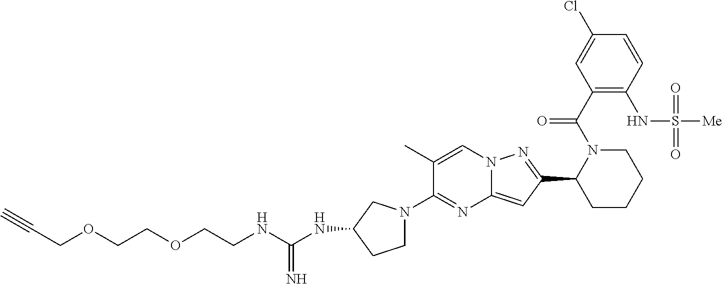 [SMILES: C#CCOCCOCCNC(=N)N[C@H]1CCN(c2nc3cc([C@@H]4CCCCN4C(=O)c4cc(Cl)ccc4NS(C)(=O)=O)nn3cc2C)C1] |
| Int-7 | 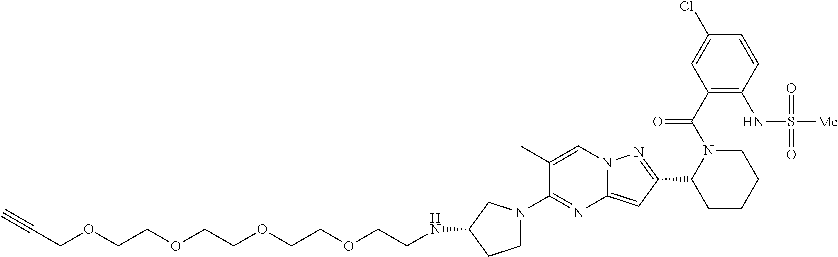 |
| Int-8 | 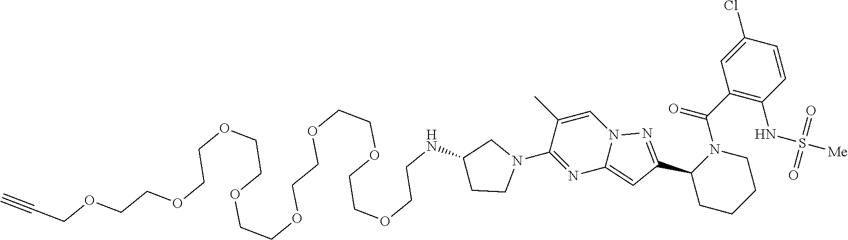 [SMILES: C#CCOCCOCCOCCOCCOCCOCCOCCOCCN[C@H]1CCN(c2nc3cc([C@@H]4CCCCN4C(=O)c4cc(Cl)ccc4NS(C)(=O)=O)nn3cc2C)C1] |
| Int-9 | 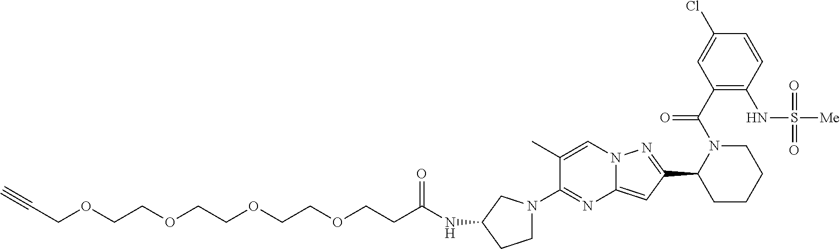 [SMILES: C#CCOCCOCCOCCOCCC(=O)N[C@H]1CCN(c2nc3cc([C@@H]4CCCCN4C(=O)c4cc(Cl)ccc4NS(C)(=O)=O)nn3cc2C)C1] |
| Int-10 | 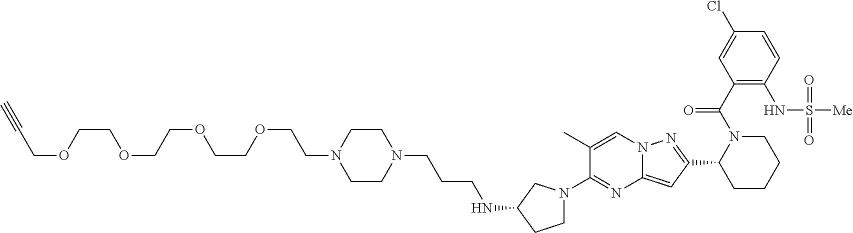 [SMILES: C#CCOCCOCCOCCOCCN1CCN(CCCN[C@H]2CCN(c3nc4cc([C@H]5CCCCN5C(=O)c5cc(Cl)ccc5NS(C)(=O)=O)nn4cc3C)C2)CC1] |
| Int-23 | 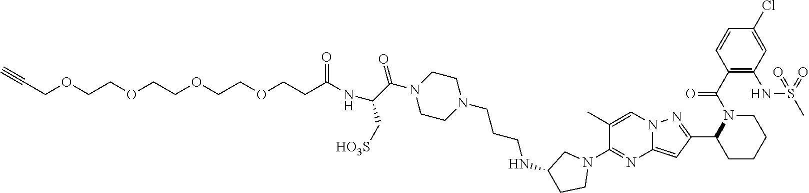 [SMILES: C#CCOCCOCCOCCOCCC(=O)N[C@@H](CS(=O)(=O)O)C(=O)N1CCN(CCCN[C@H]2CCN(c3nc4cc([C@@H]5CCCCN5C(=O)c5ccc(Cl)cc5NS(C)(=O)=O)nn4cc3C)C2)CC1] |
| Int-24 | 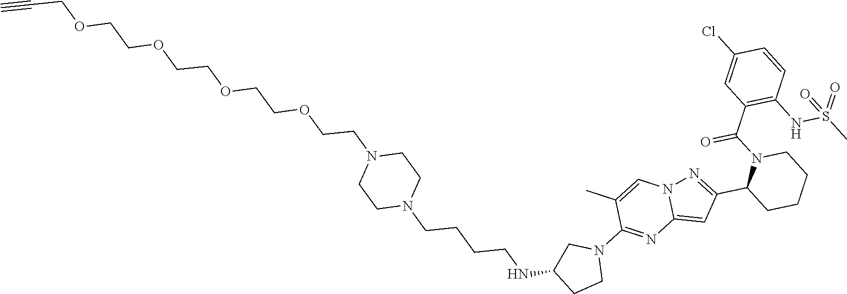 [SMILES: C#CCOCCOCCOCCOCCN1CCN(CCCCN[C@H]2CCN(c3nc4cc([C@@H]5CCCCN5C(=O)c5cc(Cl)ccc5NS(C)(=O)=O)nn4cc3C)C2)CC1] |
| Int-25 | 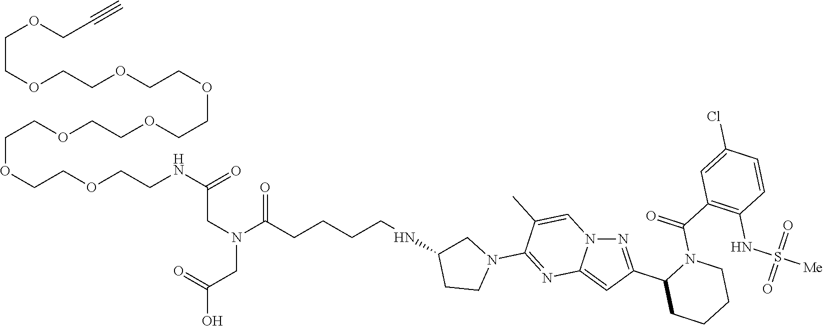 [SMILES: C#CCOCCOCCOCCOCCOCCOCCOCCOCCNC(=O)CN(CC(=O)O)C(=O)CCCCN[C@H]1CCN(c2nc3cc([C@@H]4CCCCN4C(=O)c4cc(Cl)ccc4NS(C)(=O)=O)nn3cc2C)C1] |
| Int-26 | 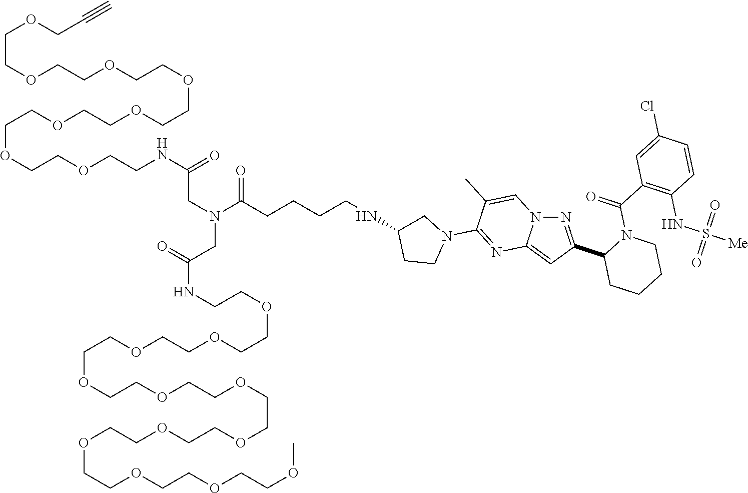 |
| Int-27 | 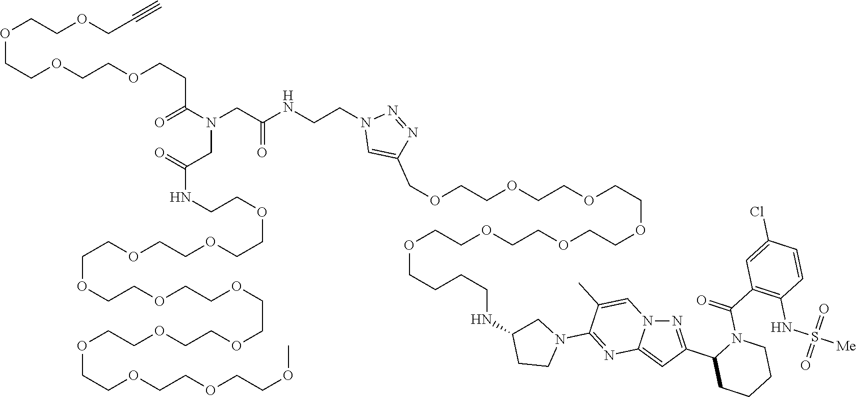 |
| Int-28 | 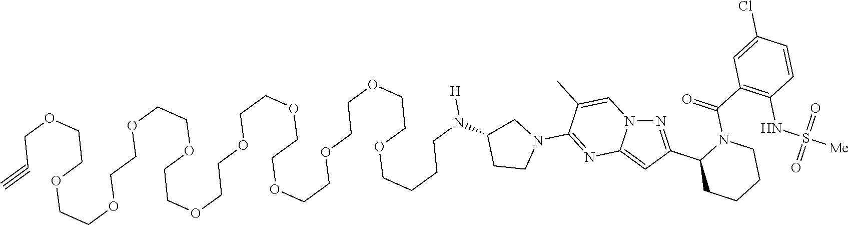 [SMILES: C#CCOCCOCCOCCOCCOCCOCCOCCOCCOCCOCCOCCOCCCCN[C@H]1CCN(c2nc3cc([C@@H]4CCCCN4C(=O)c4cc(Cl)ccc4NS(C)(=O)=O)nn3cc2C)C1] |
| Int-29 | 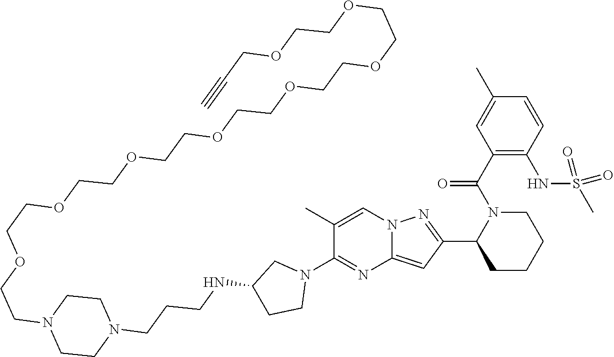 [SMILES: C#CCOCCOCCOCCOCCOCCOCCOCCOCCN1CCN(CCCN[C@H]2CCN(c3nc4cc([C@@H]5CCCCN5C(=O)c5cc(C)ccc5NS(C)(=O)=O)nn4cc3C)C2)CC1] |
| Int-34 | 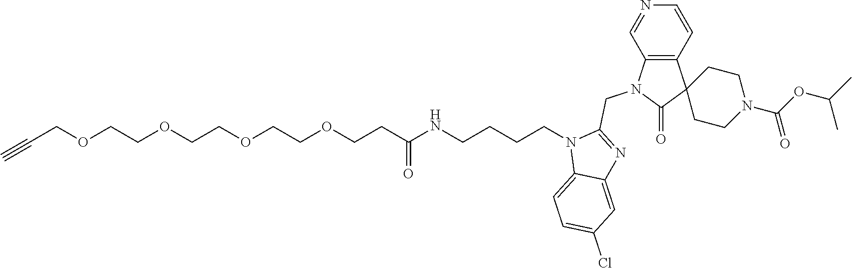 [SMILES: C#CCOCCOCCOCCOCCC(=O)NCCCCn1c(CN2C(=O)C3(CCN(C(=O)OC(C)C)CC3)c3ccncc32)nc2cc(Cl)ccc21] |
| Int-35 | 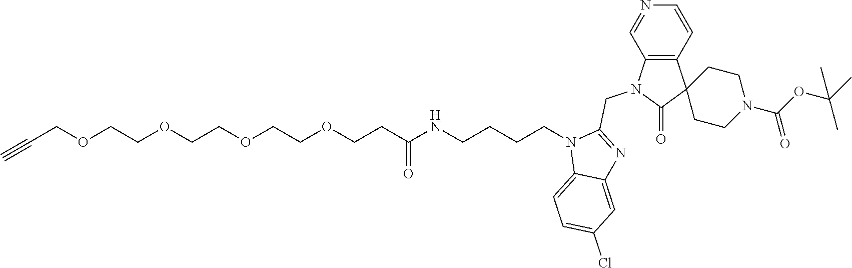 [SMILES: C#CCOCCOCCOCCOCCC(=O)NCCCCn1c(CN2C(=O)C3(CCN(C(=O)OC(C)(C)C)CC3)c3ccncc32)nc2cc(Cl)ccc21] |
| Int-36 | 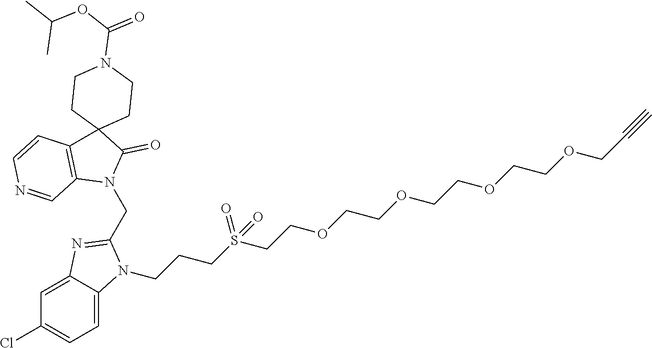 [SMILES: C#CCOCCOCCOCCOCCS(=O)(=O)CCCn1c(CN2C(=O)C3(CCN(C(=O)OC(C)C)CC3)c3ccncc32)nc2cc(Cl)ccc21] |
| Int-37 | 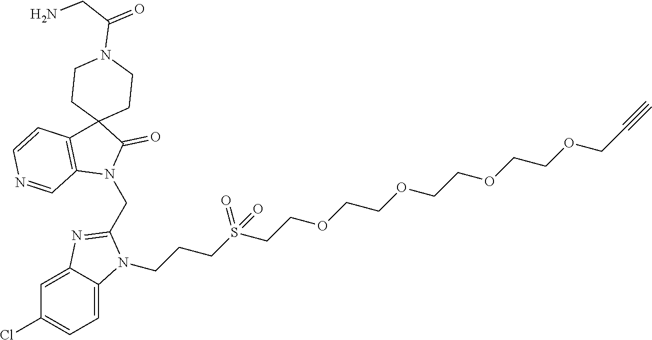 [SMILES: C#CCOCCOCCOCCOCCS(=O)(=O)CCCn1c(CN2C(=O)C3(CCN(C(=O)CN)CC3)c3ccncc32)nc2cc(Cl)ccc21] |
| Int-38 | 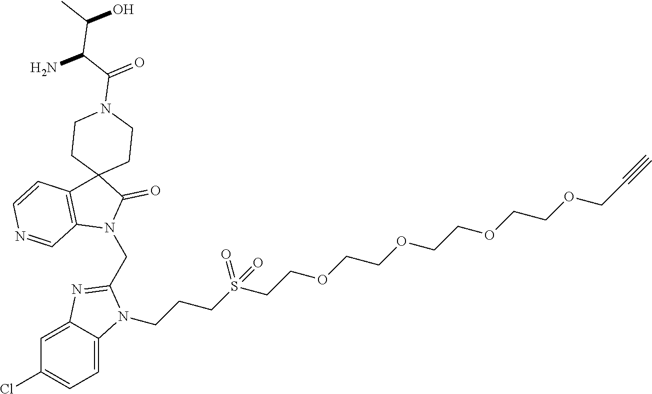 [SMILES: C#CCOCCOCCOCCOCCS(=O)(=O)CCCn1c(CN2C(=O)C3(CCN(C(=O)[C@@H](N)[C@@H](C)O)CC3)c3ccncc32)nc2cc(Cl)ccc21] |
| Int-39 | 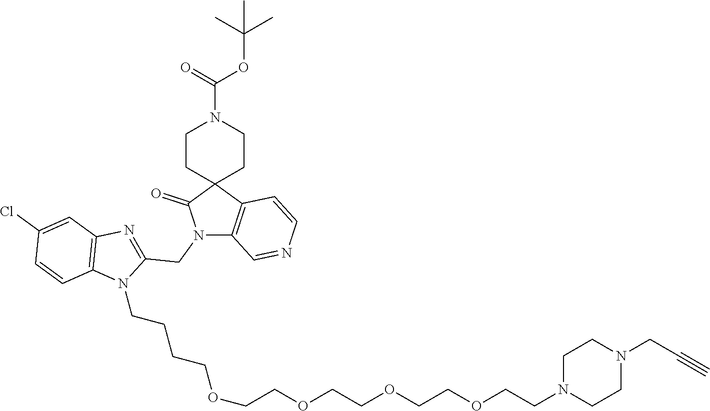 [SMILES: C#CCN1CCN(CCOCCOCCOCCOCCCCn2c(CN3C(=O)C4(CCN(C(=O)OC(C)(C)C)CC4)c4ccncc43)nc3cc(Cl)ccc32)CC1] |
| Int-40 | 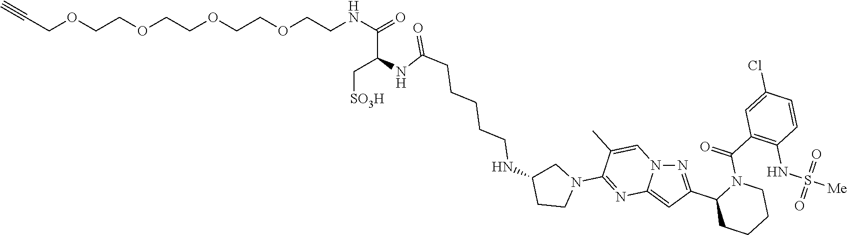 [SMILES: C#CCOCCOCCOCCOCCNC(=O)[C@H](CS(=O)(=O)O)NC(=O)CCCCCN[C@H]1CCN(c2nc3cc([C@@H]4CCCCN4C(=O)c4cc(Cl)ccc4NS(C)(=O)=O)nn3cc2C)C1] |
| Int-41 | 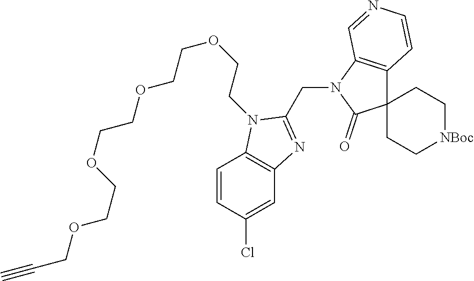 [SMILES: C#CCOCCOCCOCCOCCn1c(CN2C(=O)C3(CCN(C(=O)OC(C)(C)C)CC3)c3ccncc32)nc2cc(Cl)ccc21] |
| Int-42 | 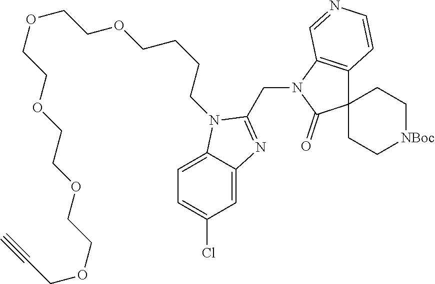 [SMILES: C#CCOCCOCCOCCOCCOCCCCn1c(CN2C(=O)C3(CCN(C(=O)OC(C)(C)C)CC3)c3ccncc32)nc2cc(Cl)ccc21] |
| Int-43 | 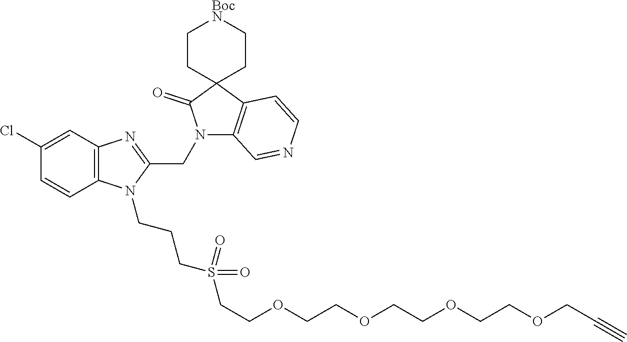 [SMILES: C#CCOCCOCCOCCOCCS(=O)(=O)CCCn1c(CN2C(=O)C3(CCN(C(=O)OC(C)(C)C)CC3)c3ccncc32)nc2cc(Cl)ccc21] |
| Int-44 | 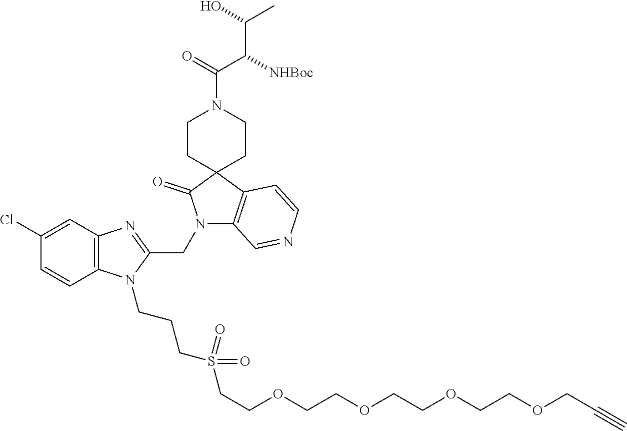 [SMILES: C#CCOCCOCCOCCOCCS(=O)(=O)CCCn1c(CN2C(=O)C3(CCN(C(=O)[C@@H](NC(=O)OC(C)(C)C)[C@@H](C)O)CC3)c3ccncc32)nc2cc(Cl)ccc21] |
| Int-45 | 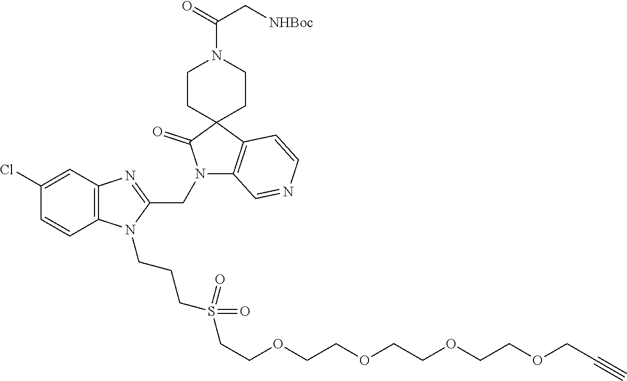 [SMILES: C#CCOCCOCCOCCOCCS(=O)(=O)CCCn1c(CN2C(=O)C3(CCN(C(=O)CNC(=O)OC(C)(C)C)CC3)c3ccncc32)nc2cc(Cl)ccc21] |
| Int-46 | 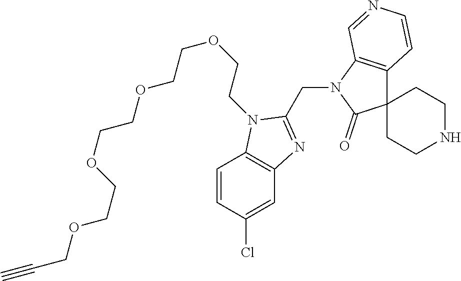 [SMILES: C#CCOCCOCCOCCOCCn1c(CN2C(=O)C3(CCNCC3)c3ccncc32)nc2cc(Cl)ccc21] |
| Int-47 | 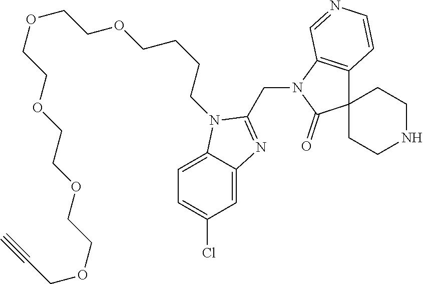 [SMILES: C#CCOCCOCCOCCOCCOCCCCn1c(CN2C(=O)C3(CCNCC3)c3ccncc32)nc2cc(Cl)ccc21] |
| Int-48 | 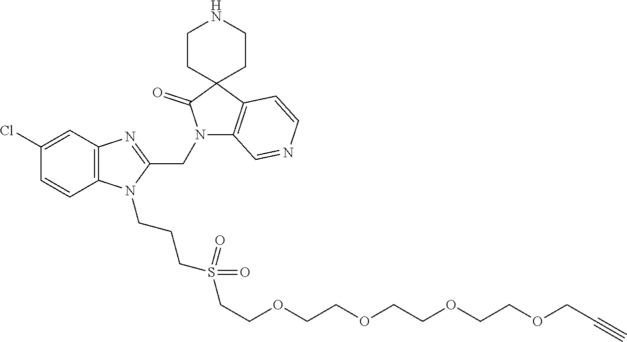 [SMILES: C#CCOCCOCCOCCOCCS(=O)(=O)CCCn1c(CN2C(=O)C3(CCNCC3)c3ccncc32)nc2cc(Cl)ccc21] |

In another aspect, the invention features a conjugate of Table 2. Each conjugate of Table 2 corresponds to a conjugate of either formula (M-I) or formula (D-I), as indicated. Conjugates of Table 2 include conjugates formed by the covalent reaction of an Int of Table 1 with a linker which is in turn conjugated to E (e.g., an Fc domain monomer, an albumin protein, an albumin protein-binding peptide, or an Fc-binding peptide). In some embodiments, the reactive moiety of the Int (e.g., the alkyne or azido group) reacts with a corresponding reactive group (e.g., an alkyne or azido group) of a linker (represented by L′) covalently attached to E, such that an Int of Table 1 is covalently attached to E. As represented in Table 2, L′ corresponds to the remainder of L as defined in (M-I) or (D-I) (e.g., L′ is a linker that covalently joins the Int and E). For example, L′ may include a triazole (formed by the click chemistry reaction between the Int and a linker conjugated to E) and a linker (e.g., a PEG₂-PEG₂₀ linker) which in turn is conjugated to an amino acid side chain of E.

In some embodiments in any conjugate of Table 2, n is 1 or 2. When n is 1, each E includes an Fc domain monomer (e.g., an Fc domain monomer having the sequence of any one of SEQ ID NOs: 1-95), an albumin protein (e.g., an albumin protein having the sequence of any one of SEQ ID NOs: 96-98), an albumin protein-binding peptide, or an Fc-binding peptide. When n is 2, each E includes an Fc domain monomer (e.g., an Fc domain monomer having the sequence of any one of SEQ ID NOs: 1-95), and the Fc domain monomers dimerize to form and Fc domain.

In some embodiments in any conjugate of Table 2, T is an integer from 1 to 20 (e.g., 1, 2, 3, 4, 5, 6, 7, 8, 9, 10, 11, 12, 13, 14, 15, 16, 17, 18, 19, or 20). The disclosure also provides a population of any of the conjugates of Table 2 wherein the average value of T is 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20.

The squiggly line in the conjugates of Table 2 indicates that each L′-Int is covalently attached to an amino acid side chain in E (e.g., the nitrogen atom of a surface exposed lysine or the sulfur atom of a surface exposed cysteine in E), or a pharmaceutically acceptable salt thereof.

TABLE 2
Conjugates Corresponding to Intermediates of Table 1

| Corre- | |
|---|---|
| sponding | |
| Inter- | |
| mediate | |
| of | |
| Table 1 | Conjugate Structure |
| Int-1 | 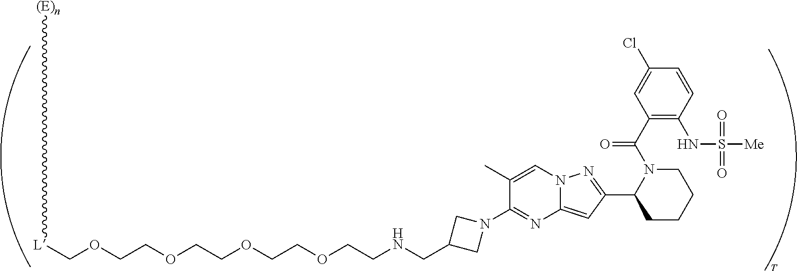 |
| Int-2 | 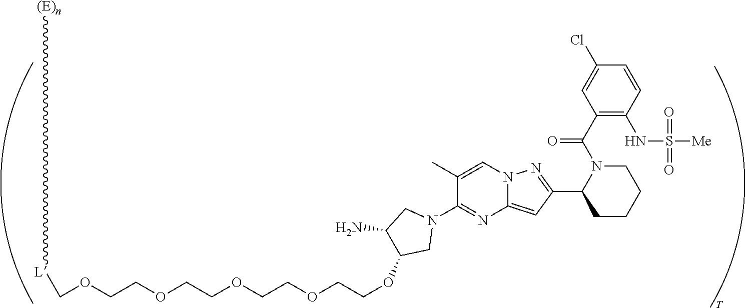 |
| Int-3 | 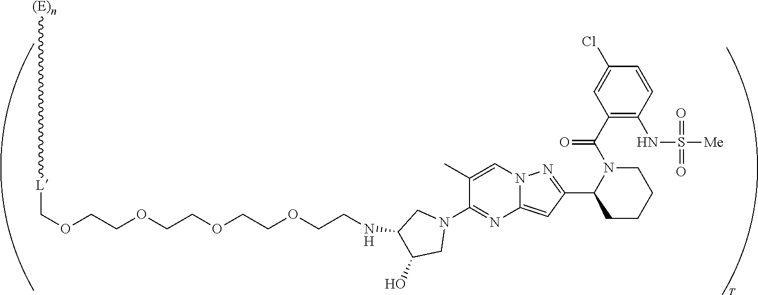 |
| Int-4 | 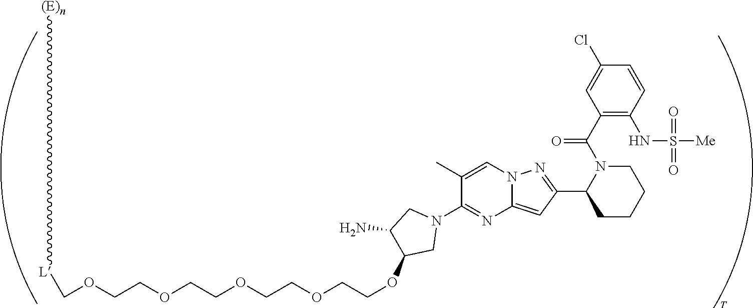 |
| Int-5 | 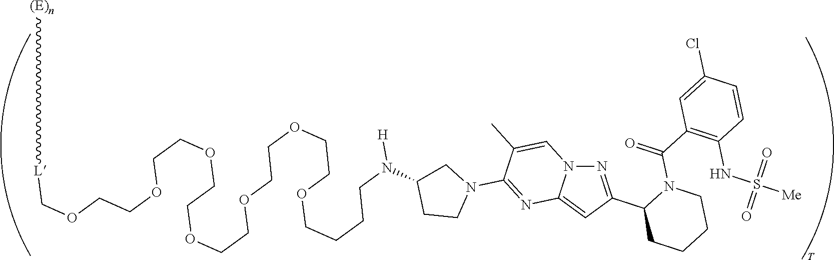 |
| Int-6 | 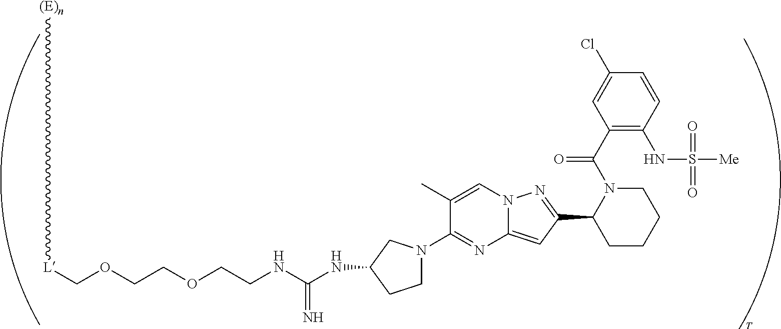 |
| Int-7 | 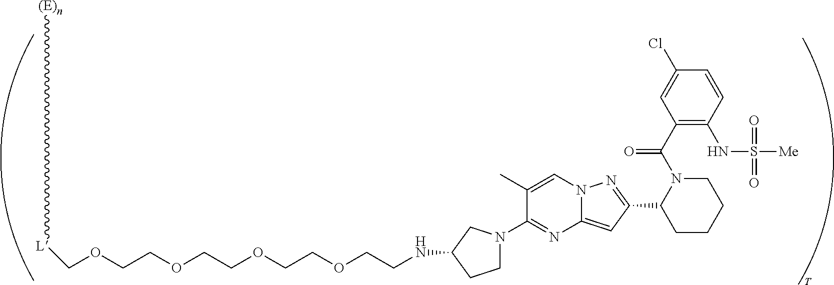 |
| Int-8 | 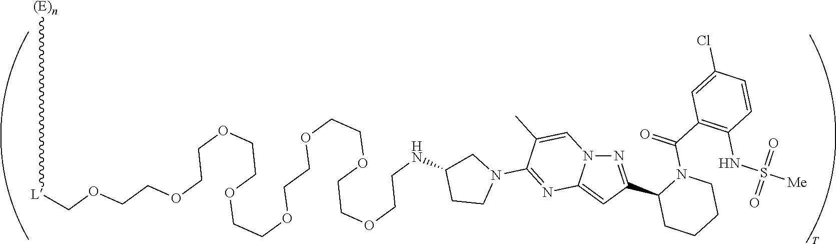 |
| Int-9 | 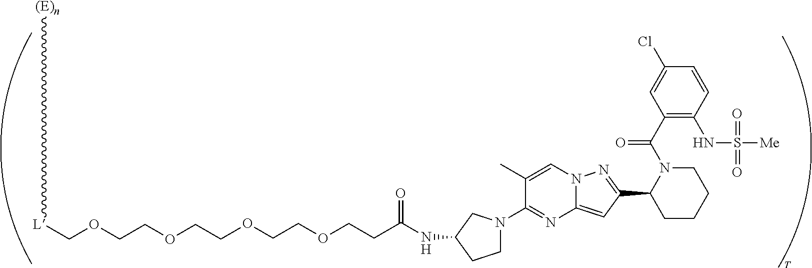 |
| Int-10 | 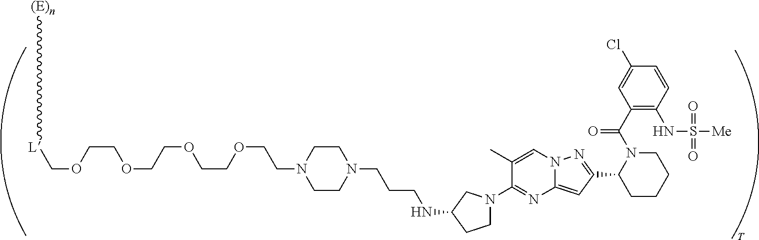 |
| Int-23 | 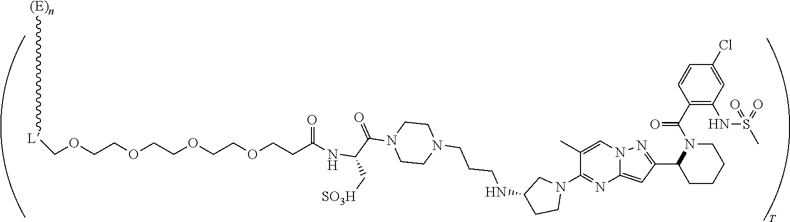 |
| Int-24 | 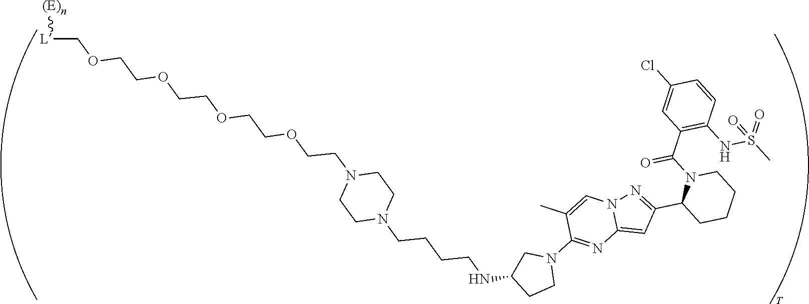 |
| Int-25 | 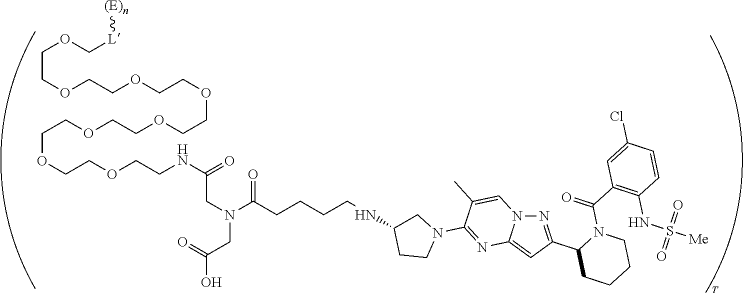 |
| Int-26 | 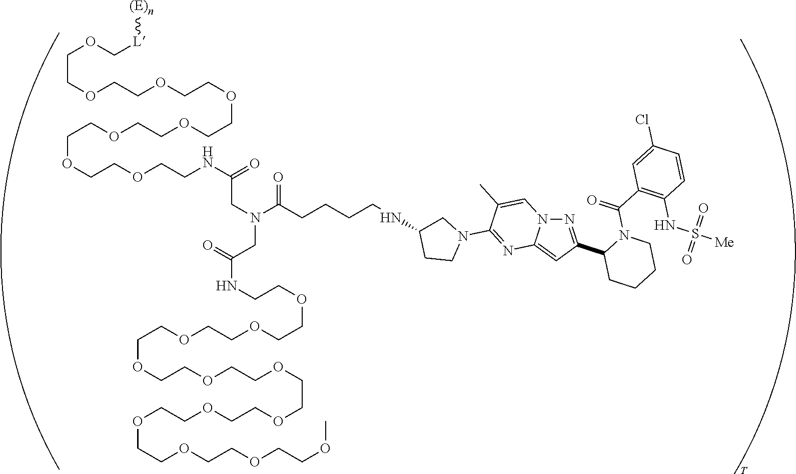 |
| Int-27 | 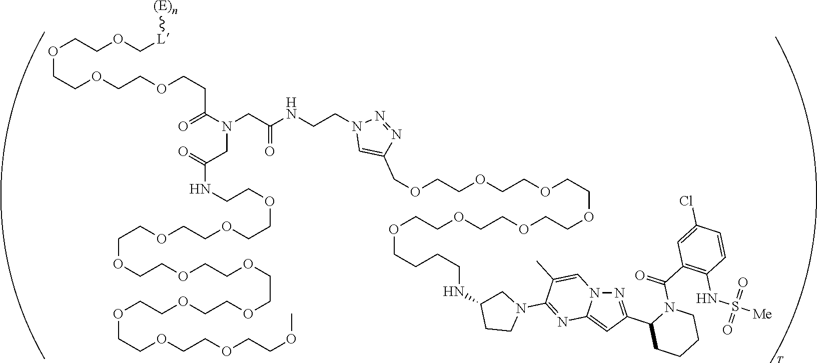 |
| Int-28 | 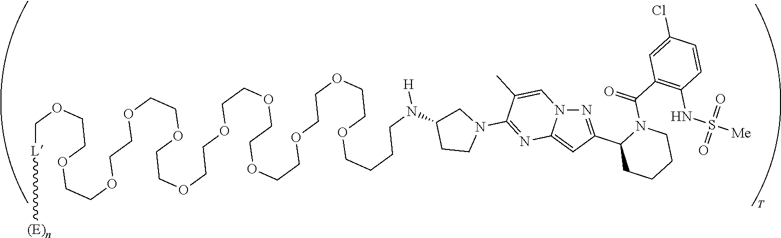 |
| Int-29 | 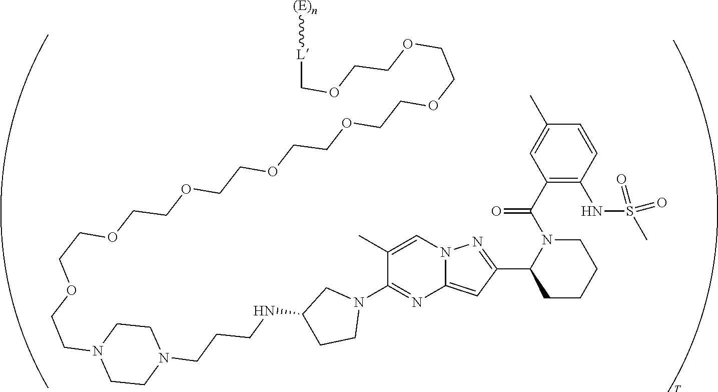 |
| Int-34 | 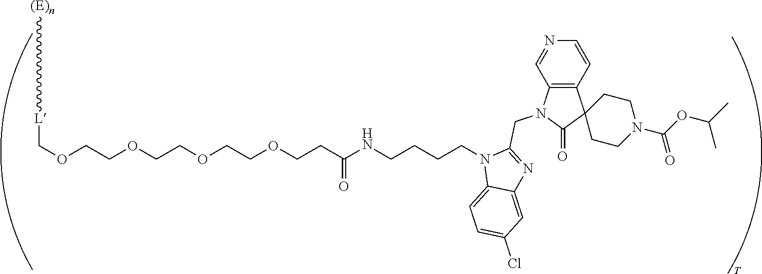 |
| Int-35 | 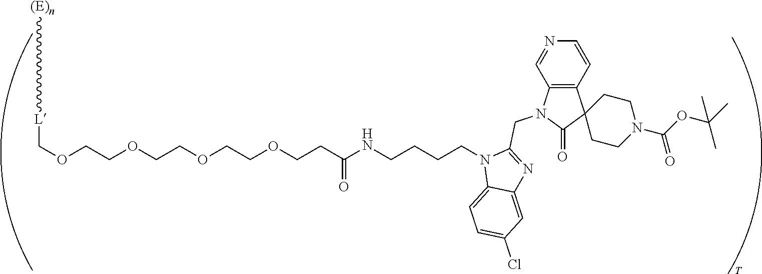 |
| Int-36 | 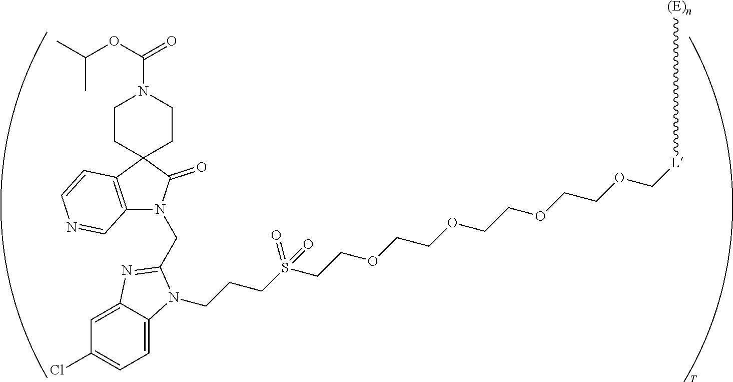 |
| Int-37 | 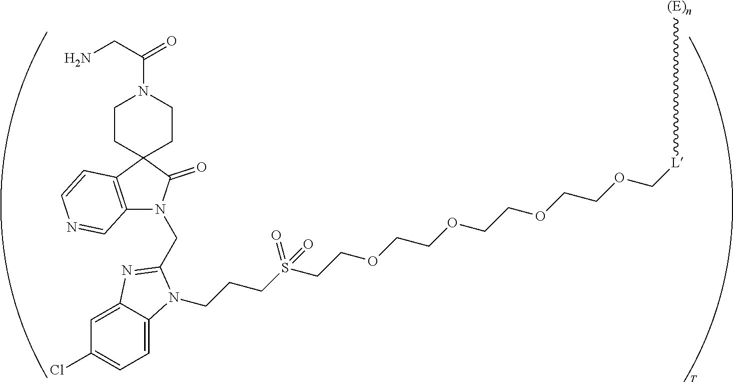 |
| Int-38 | 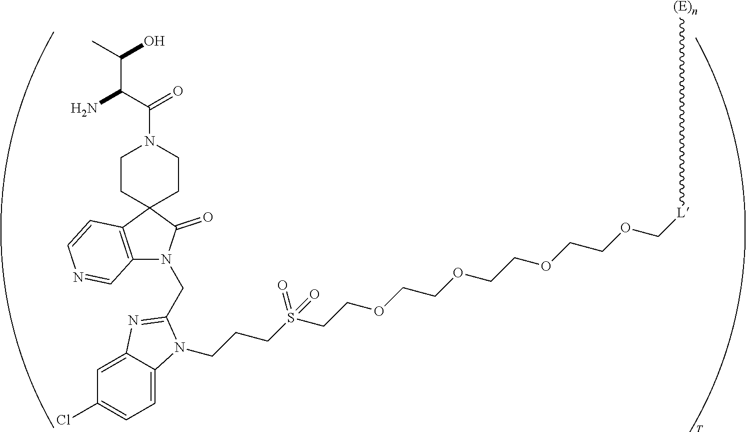 |
| Int-39 | 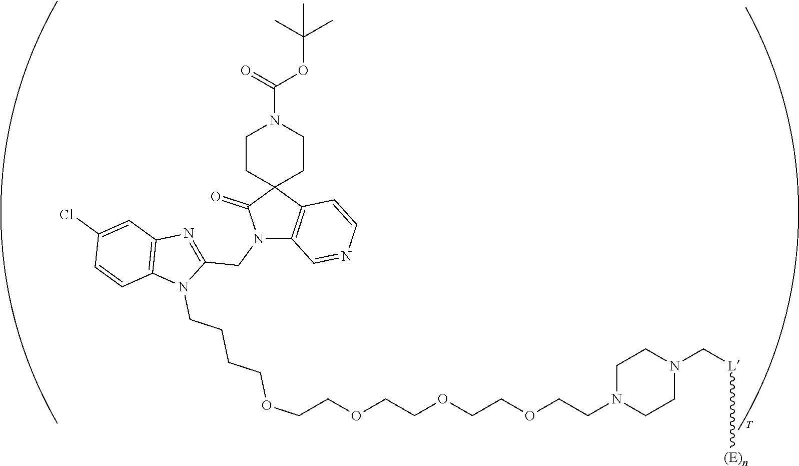 |
| Int-40 | 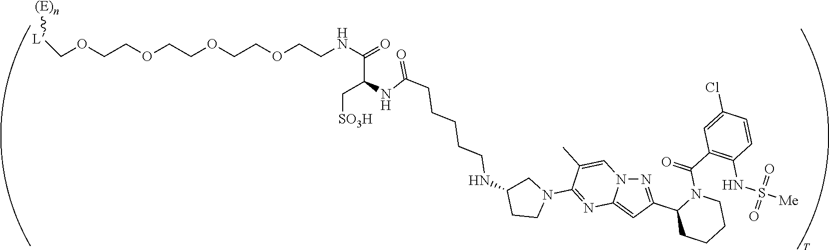 |
| Int-41 | 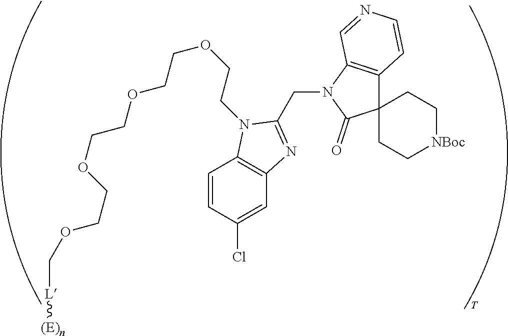 |
| Int-42 | 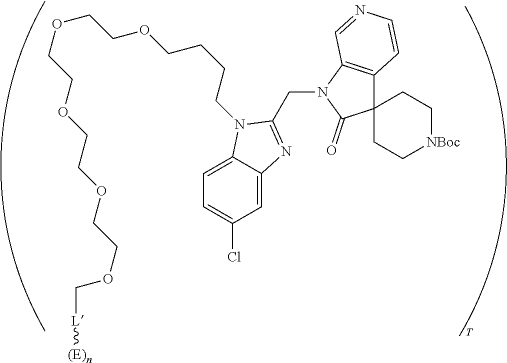 |
| Int-43 | 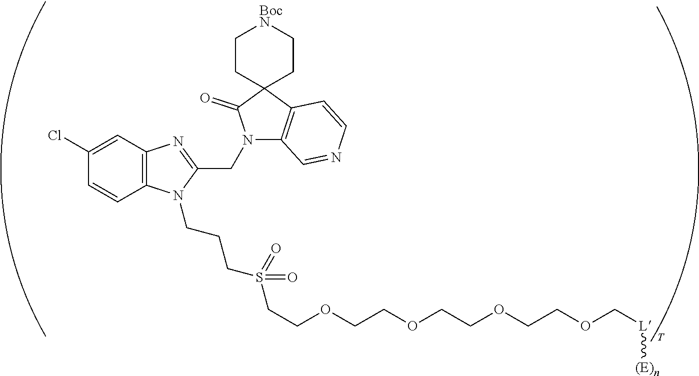 |
| Int-44 | 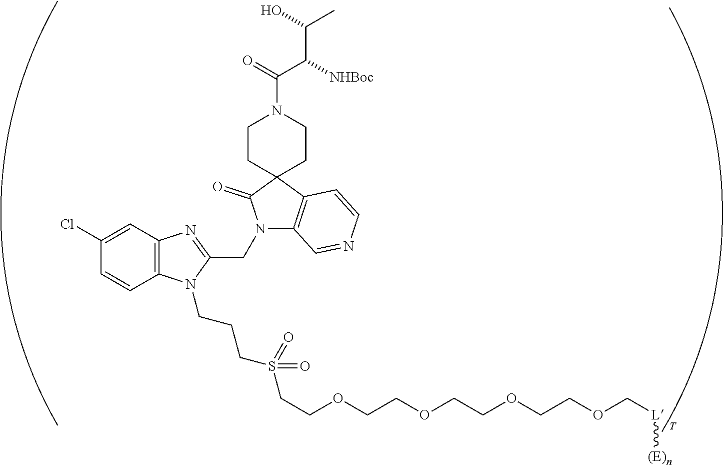 |
| Int-45 | 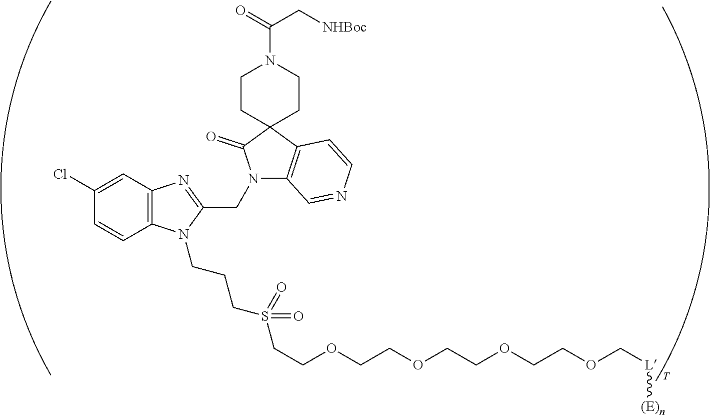 |
| Int-46 | 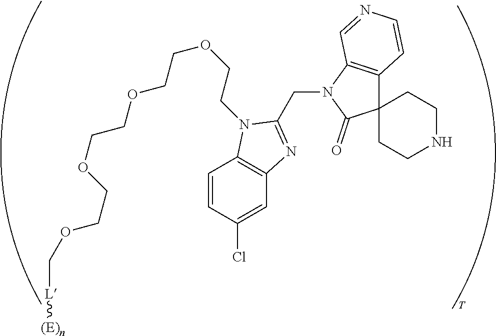 |
| Int-47 | 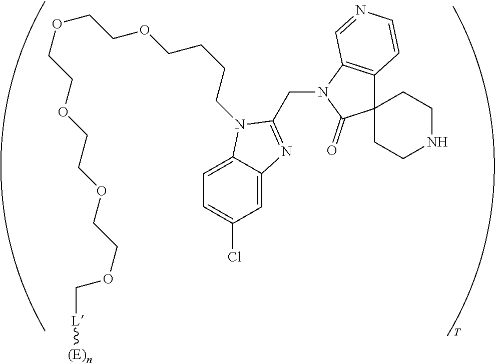 |
| Int-48 | 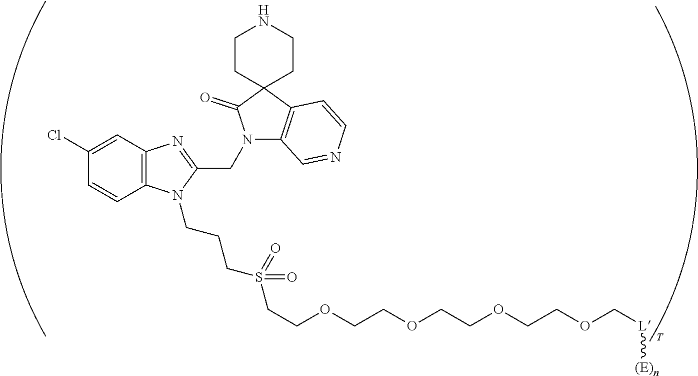 |

In another aspect, the invention features a conjugate including (i) a first moiety, A₁; (ii) a second moiety, A₂; (iii) an Fc domain monomer or an Fc domain; and (iv) a linker covalently attached to A₁ and A₂, and to the Fc domain monomer or the Fc domain; wherein each A₁ and each A₂ is independently selected from a structure described by any one of formulas (A-I)-(A-III).

In another aspect, the invention features a conjugate including (i) a first moiety, Int; (ii) an Fc domain monomer or an Fc domain; and (iv) a linker covalently attached to Int, and to the Fc domain monomer or the Fc domain; wherein each Int is independently selected from any one of the intermediates of Table 1.

In another aspect, the invention features a conjugate including (i) a first moiety, A₁; (ii) a second moiety, A₂; (iii) an albumin protein, an albumin protein-binding peptide, or an Fc-binding peptide; and (iv) a linker covalently attached to A₁ and A₂, and to the Fc domain monomer or the Fc domain; wherein each A₁ and each A₂ is independently selected from a structure described by any one of formulas (A-I)-(A-III).

In another aspect, the invention features a conjugate described by formula (D-I):

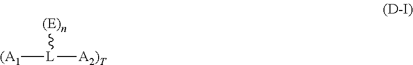

wherein each A₁ and each A₂ is independently described by formula (A-I)-(A-III).

each E independently includes an Fc domain monomer (e.g., an Fc domain monomer having the sequence of any one of SEQ ID NOs: 1-95), an albumin protein (e.g., an albumin protein having the sequence of any one of SEQ ID NOs: 96-98), an albumin protein-binding peptide, or an Fc-binding peptide; n is 1 or 2; T is an integer from 1 to 20 (e.g., T is 1, 2, 3, 4, 5, 6, 7, 8, 9, 10, 11, 12, 13, 14, 15, 16, 17, 18, 19, or 20); and L is a linker covalently attached to each of E, A₁, and A₂, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁-L-A₂ may be independently selected (e.g., independently selected from any of the A₁-L-A₂ structures described herein).

In some embodiments, the conjugate is described by formula (D-II):

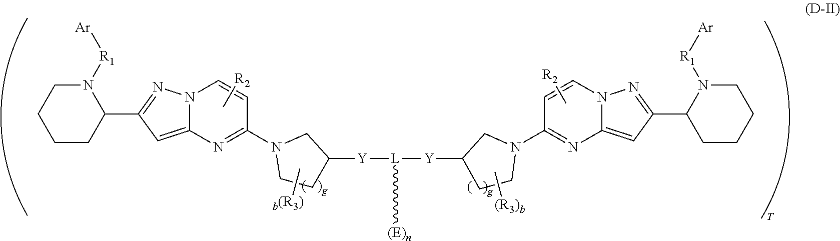

or a pharmaceutically acceptable salt thereof.

In some embodiments, conjugate is described by formula (D-II-1):

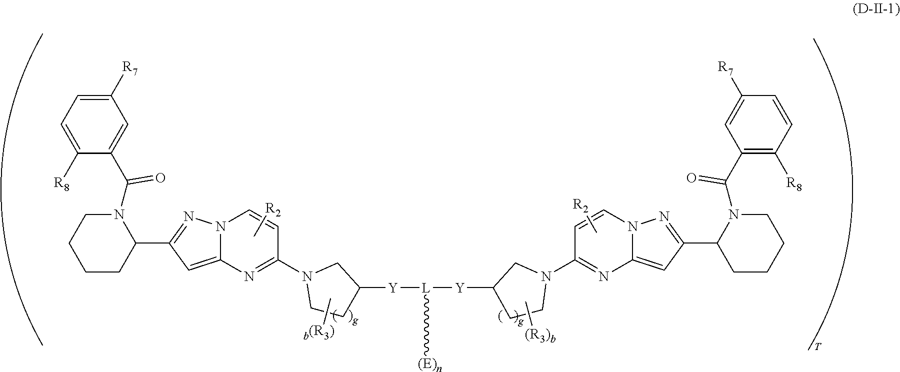

wherein R₇ and R₈ are each independently selected from OH, halogen, nitrile, nitro, optionally substituted amine, optionally substituted imine, optionally substituted C₁-C₂₀ alkamino, optionally substituted sulfhydryl, optionally substituted carboxyl, optionally substituted cyano, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy; or a pharmaceutically acceptable salt thereof.

In some embodiments, the conjugate is described by formula (D-II-2):

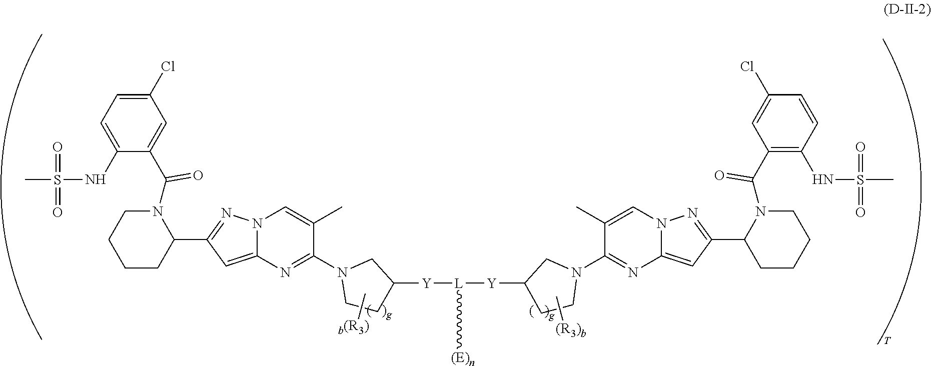

or a pharmaceutically acceptable salt thereof.

In some embodiments, the conjugate is described by the formula (D-II-3)

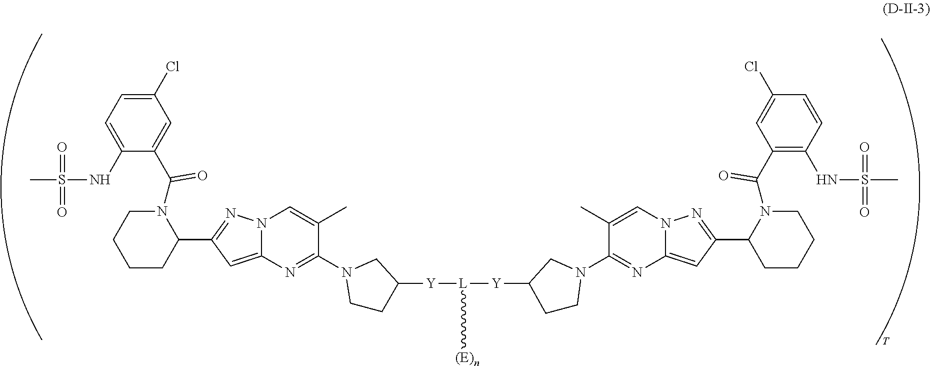

or a pharmaceutically acceptable salt thereof.

In some embodiments, the conjugate is described by the formula (D-II-4):

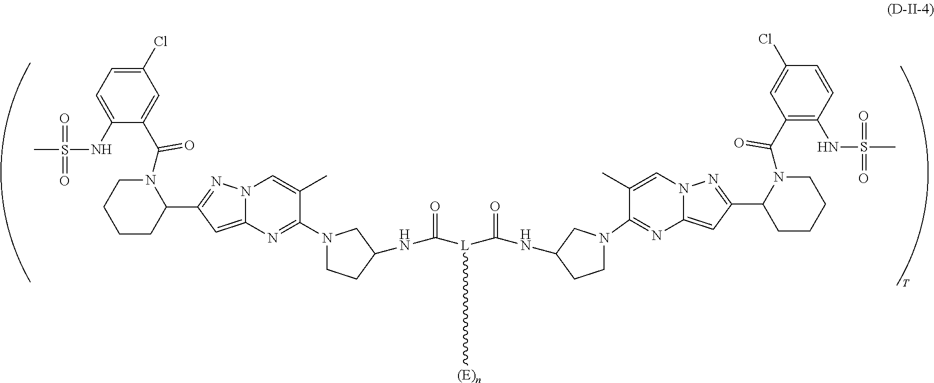

or a pharmaceutically acceptable salt thereof.

In some embodiments, the conjugate is described by the formula (D-II-5):

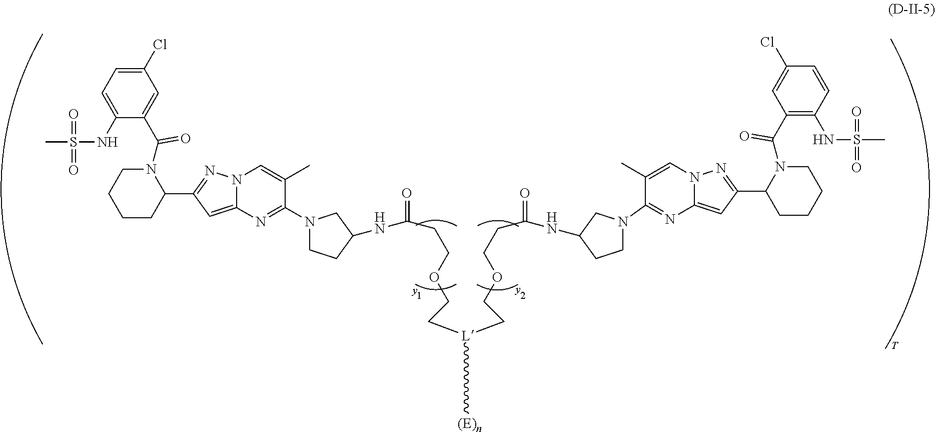

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-II-6):

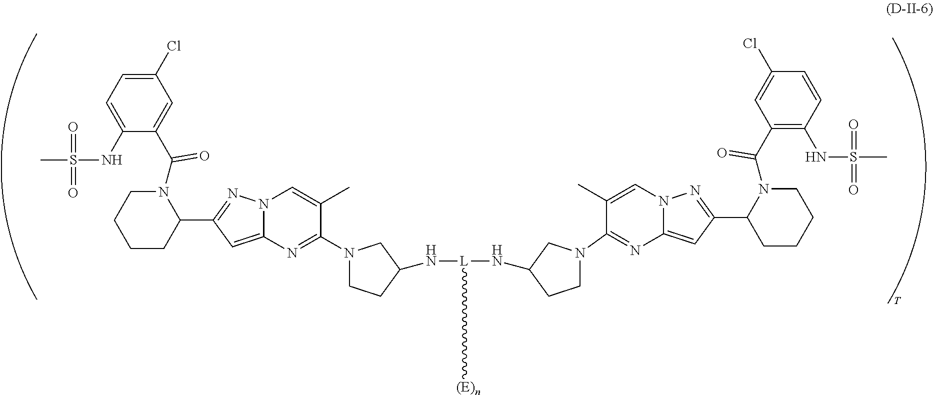

or a pharmaceutically acceptable salt thereof.

In some embodiments, the conjugate is described by the formula (D-II-7):

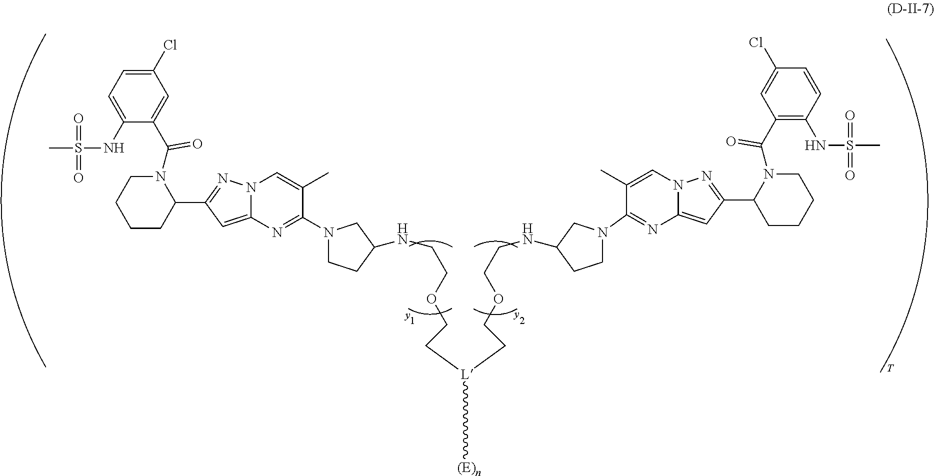

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-II-8):

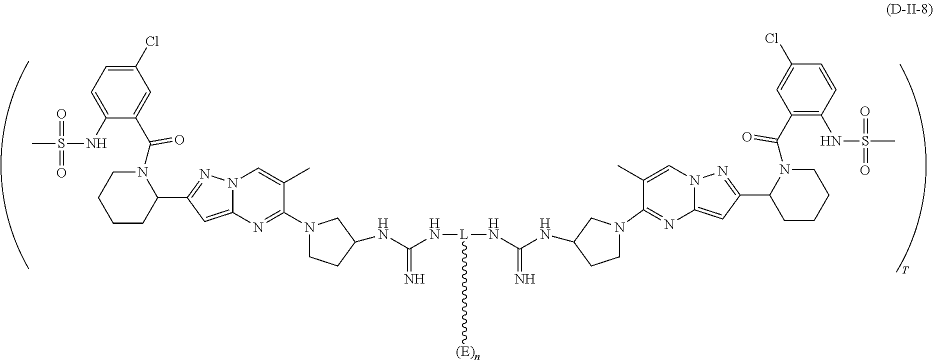

or a pharmaceutically acceptable salt thereof.

In some embodiments, the conjugate is described by formula (D-II-9):

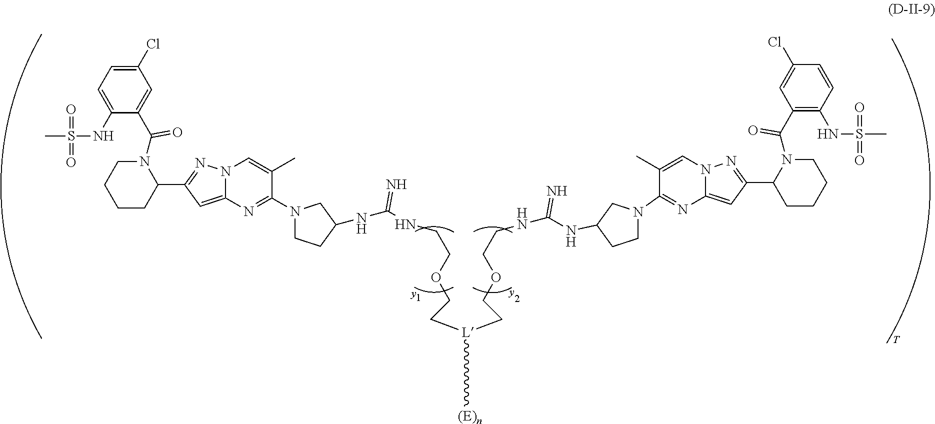

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, conjugate is described by the formula (D-II-10):

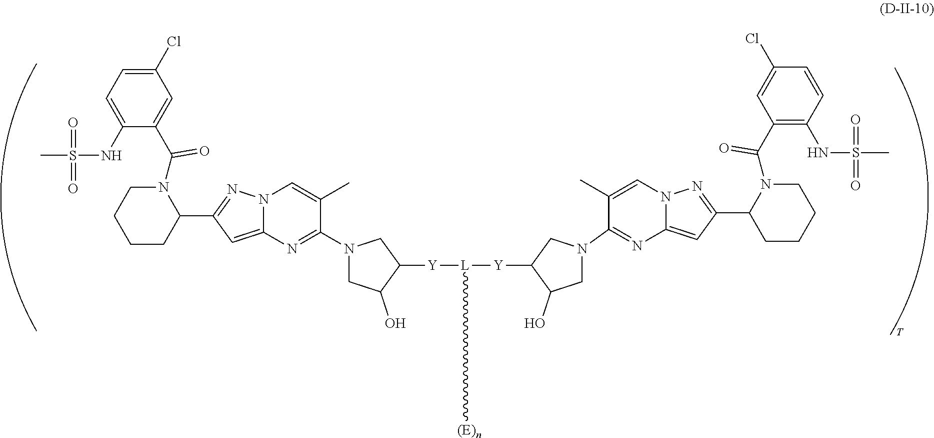

or a pharmaceutical acceptable salt thereof.

In some embodiments, the conjugate is described by formula (D-II-11):

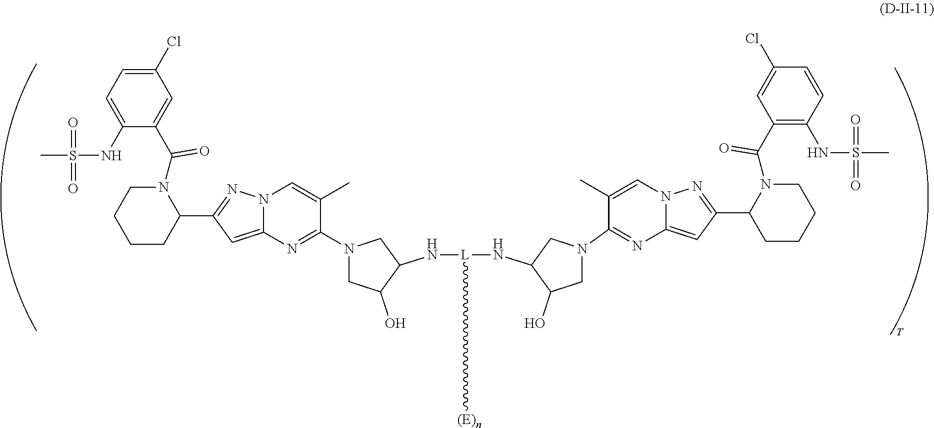

or a pharmaceutically acceptable salt thereof.

In some embodiments, the conjugate is described by the formula (D-II-12):

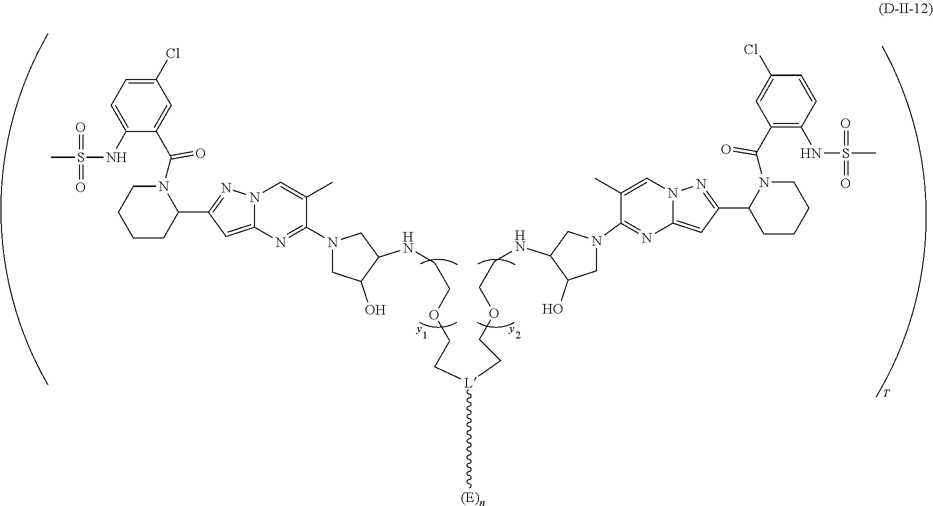

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-II-13):

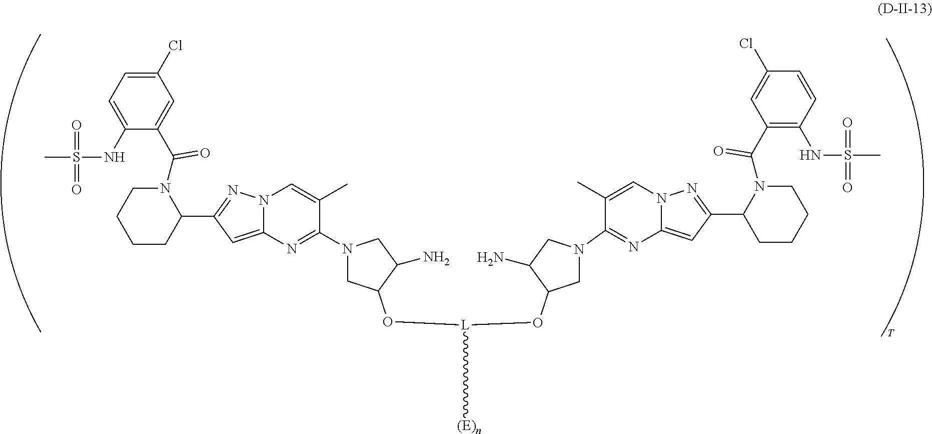

or a pharmaceutically acceptable salt thereof.

In some embodiments, the conjugate is described by formula (D-II-14):

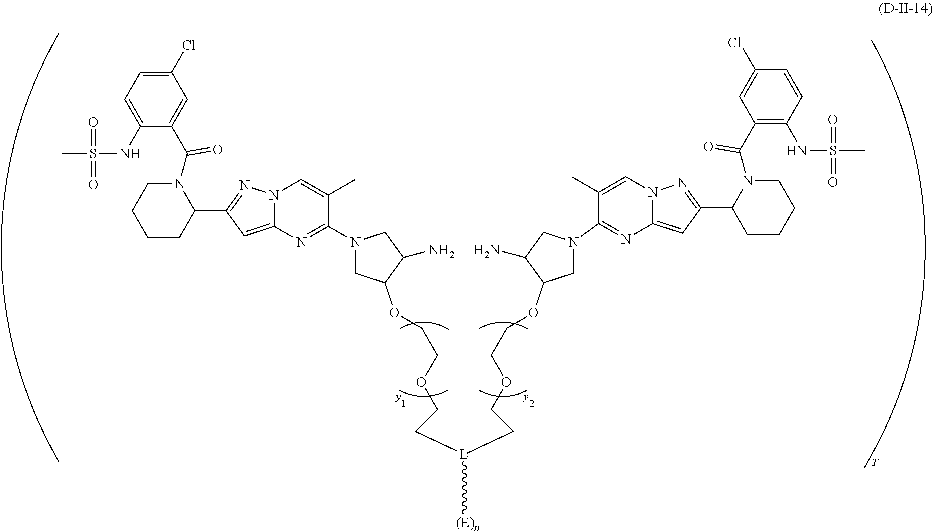

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-II-15):

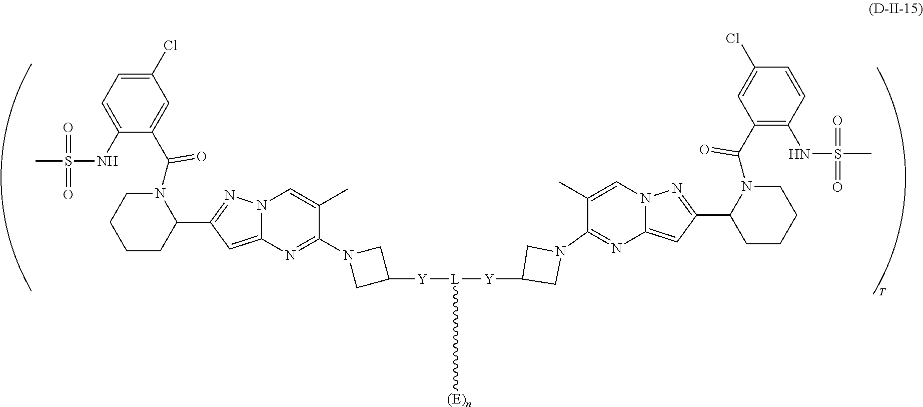

or a pharmaceutically acceptable salt thereof.

In some embodiments, the conjugate is described by formula (D-II-16):

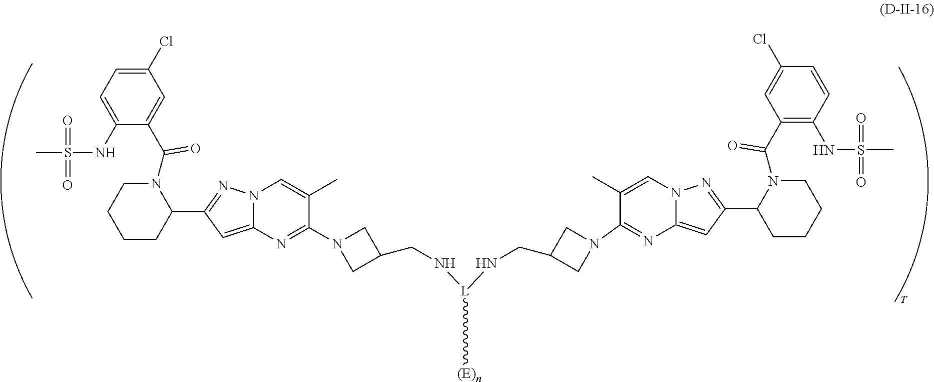

or a pharmaceutically acceptable salt thereof.

In some embodiments, the conjugate is described by formula (D-II-17):

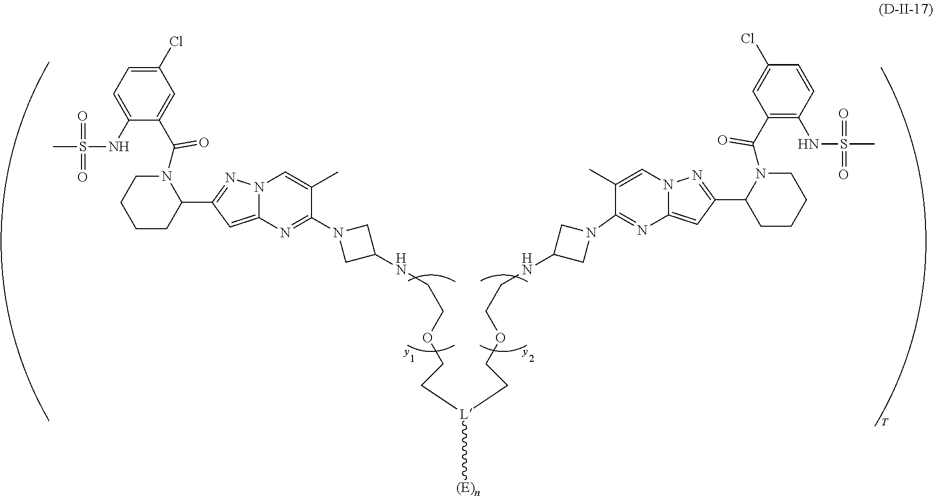

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-III):

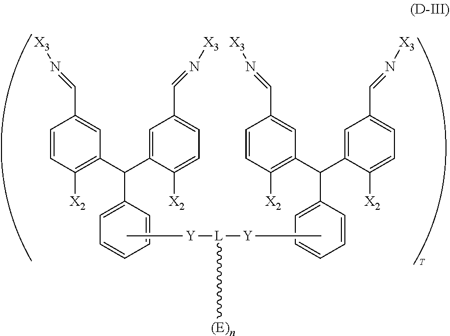

or a pharmaceutically acceptable salt thereof.

In some embodiments, the conjugate is described by formula (D-III-1):

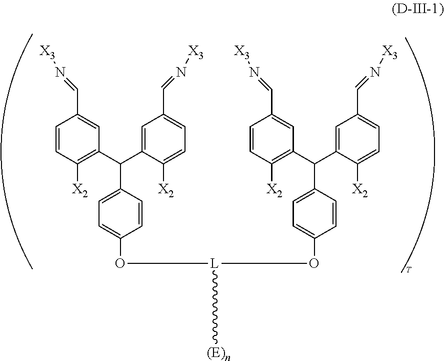

or a pharmaceutically acceptable salt thereof.

In some embodiments, the conjugate is described by formula (D-III-2):

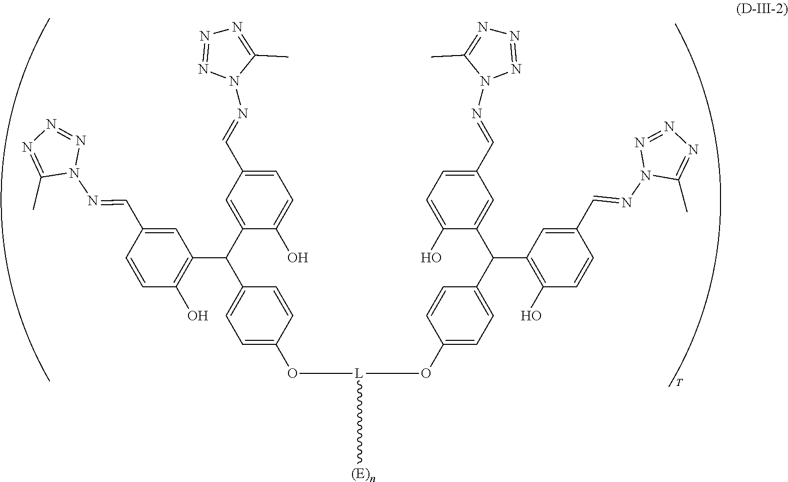

or a pharmaceutically acceptable salt thereof.

In some embodiments, the conjugate is described by formula (D-III-3):

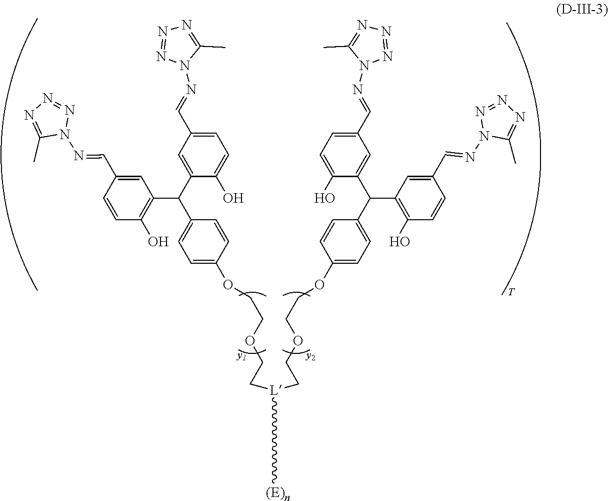

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-IV):

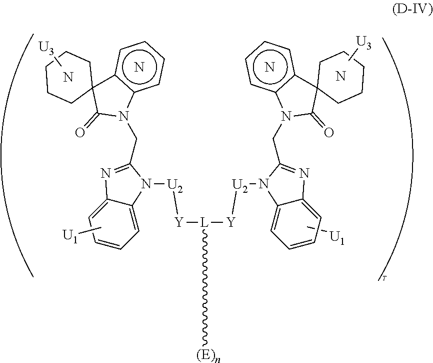

or a pharmaceutically acceptable salt thereof. In preferred embodiments, U₂ is an optionally substituted C₁-C₆ alkyl (e.g., C₁, C₂, C₃, C₄, C₅, or C₆ alkyl).

In some embodiments, the conjugate is described by formula (D-IV-1):

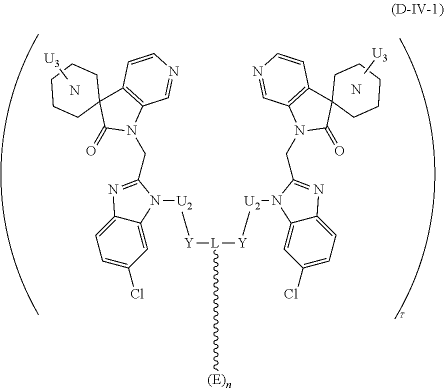

or a pharmaceutically acceptable salt thereof.

In some embodiments, the conjugate is described by formula (D-IV-2):

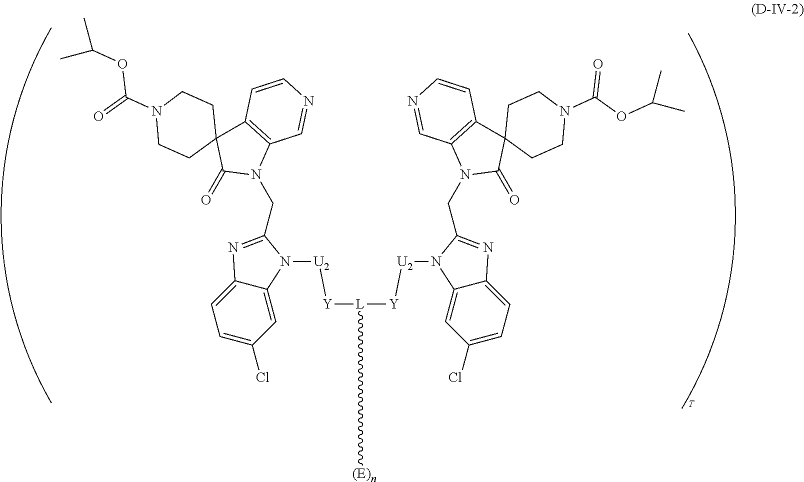

or a pharmaceutically acceptable salt thereof.

In some embodiments, the conjugate is described by formula (D-IV-3):

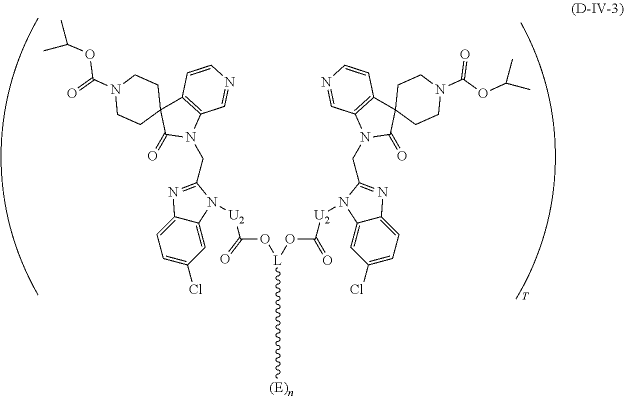

or a pharmaceutically acceptable salt thereof.

In some embodiments, the conjugate is described by formula (D-IV-4):

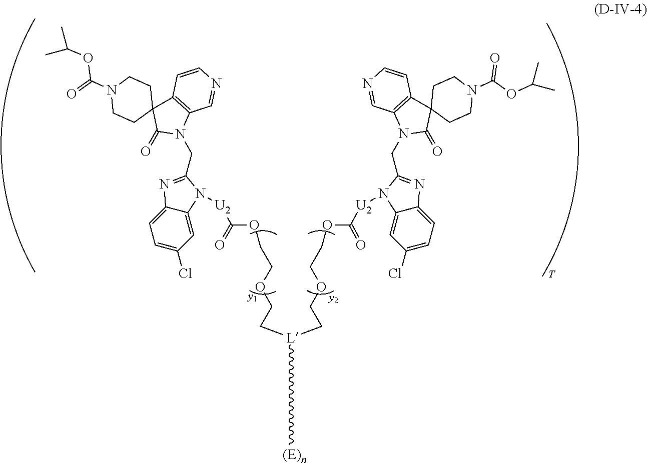

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-IV-5):

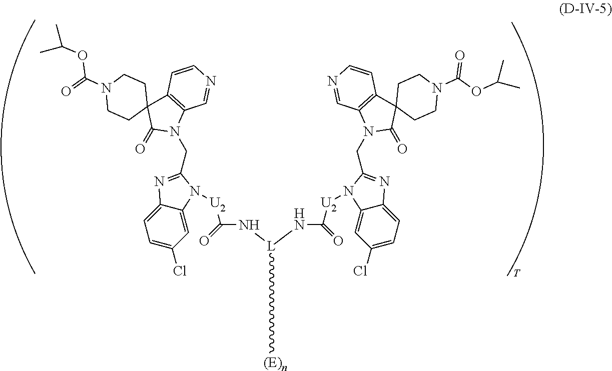

or a pharmaceutically acceptable salt thereof.

In some embodiments, the conjugate is described by formula (D-IV-6):

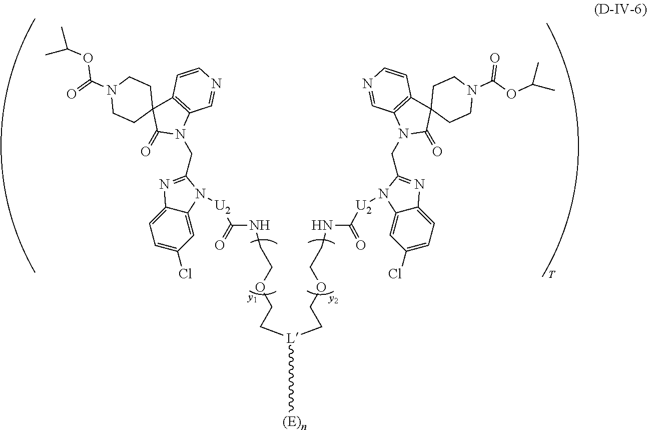

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-IV-7):

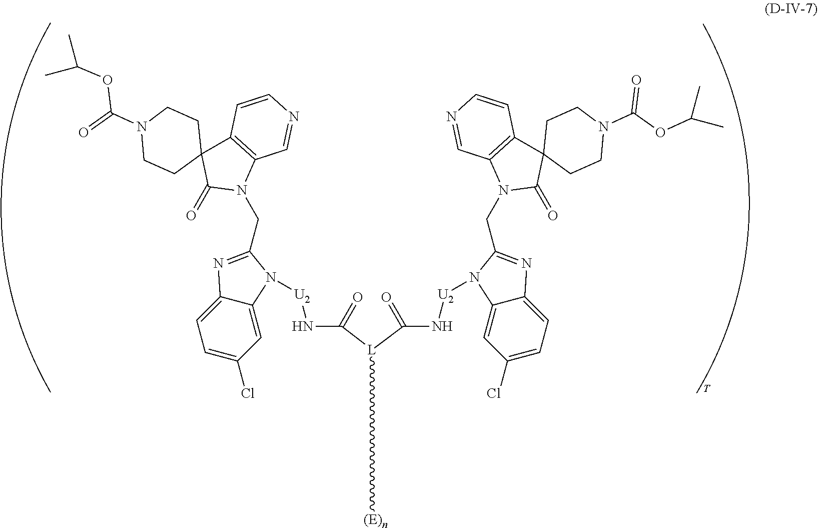

or a pharmaceutically acceptable salt thereof.

In some embodiments, the conjugate is described by formula (D-IV-8):

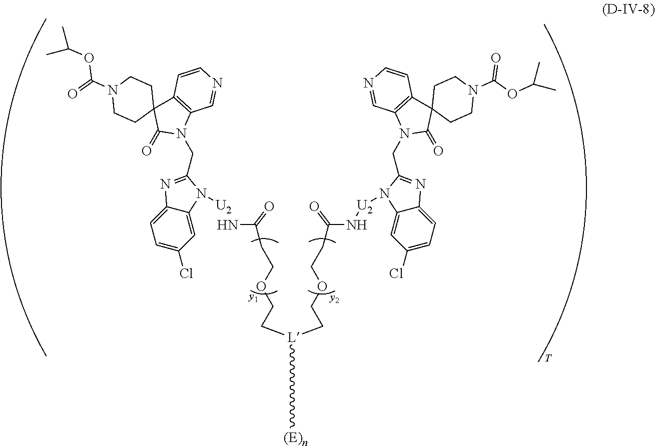

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-IV-9):

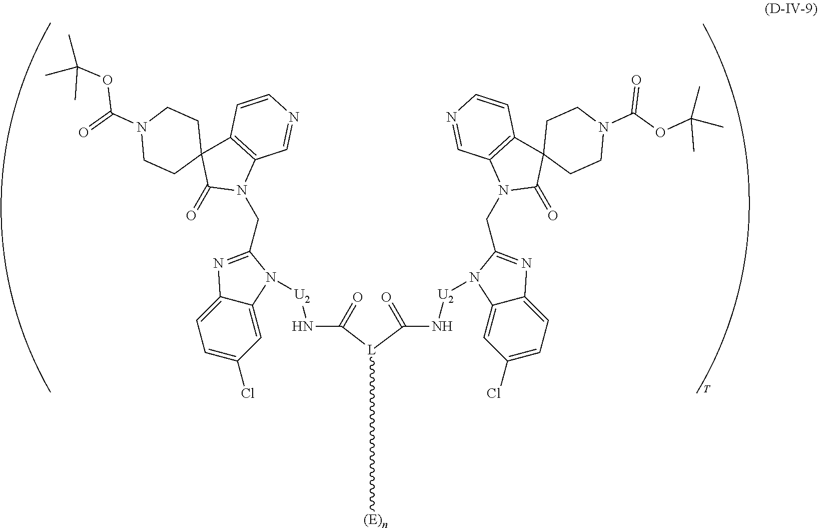

or a pharmaceutically acceptable salt thereof.

In certain embodiments, the conjugate is described by formula (D-IV-10):

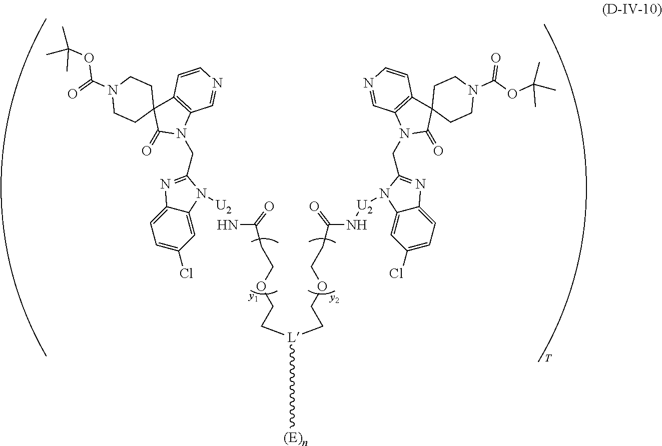

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-IV-11):

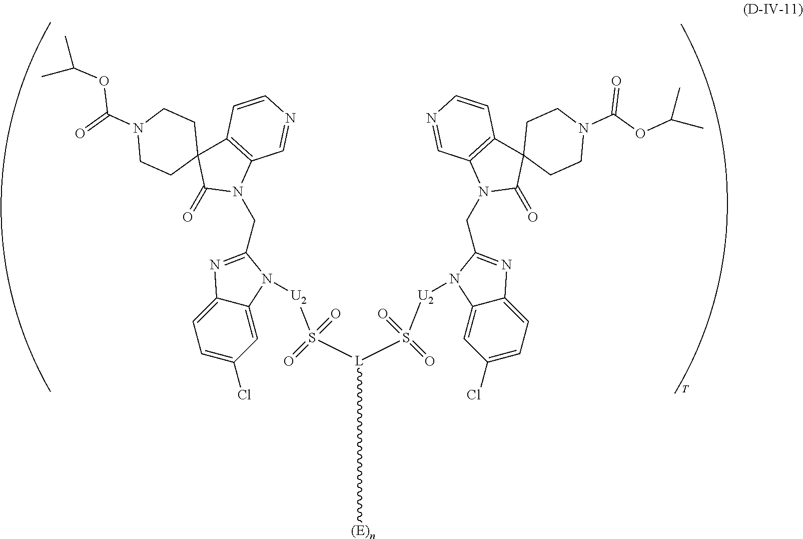

or a pharmaceutically acceptable salt thereof.

In some embodiments, the conjugate is described by formula (D-IV-12):

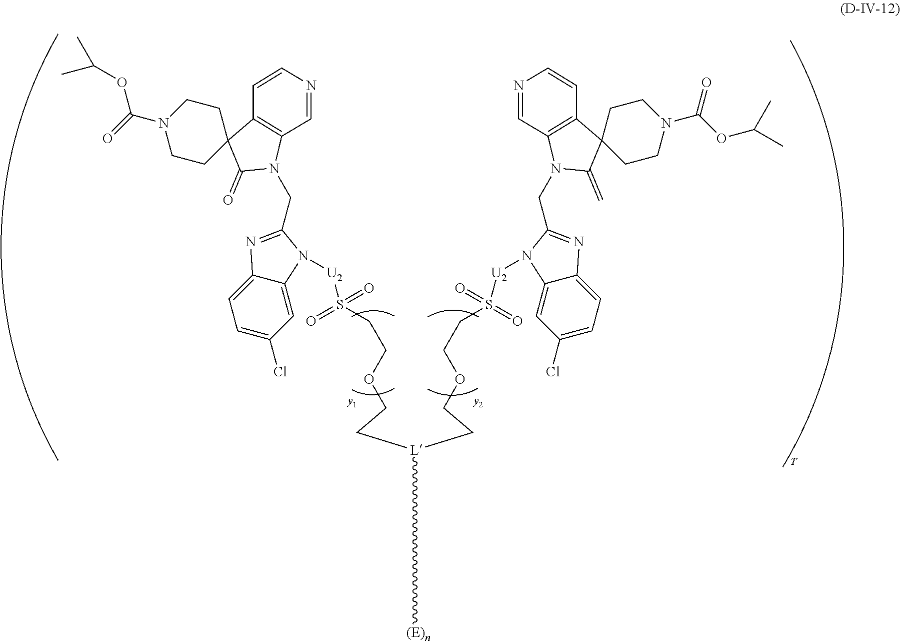

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-IV-13):

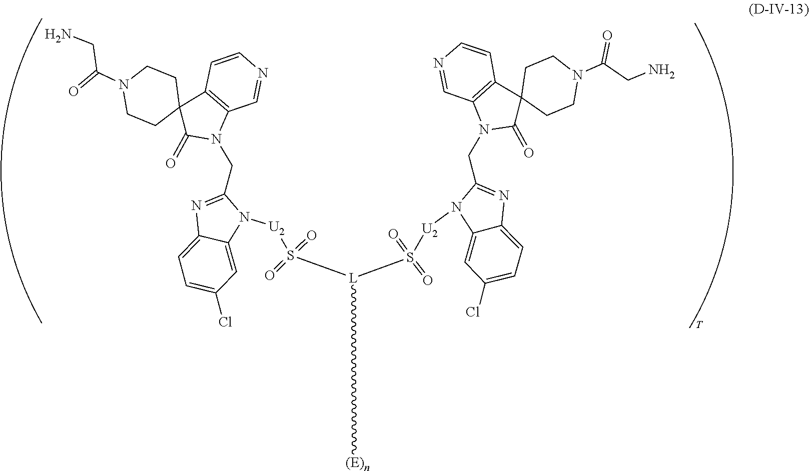

or a pharmaceutically acceptable salt thereof.

In certain embodiments, the conjugate is described by formula (D-IV-14):

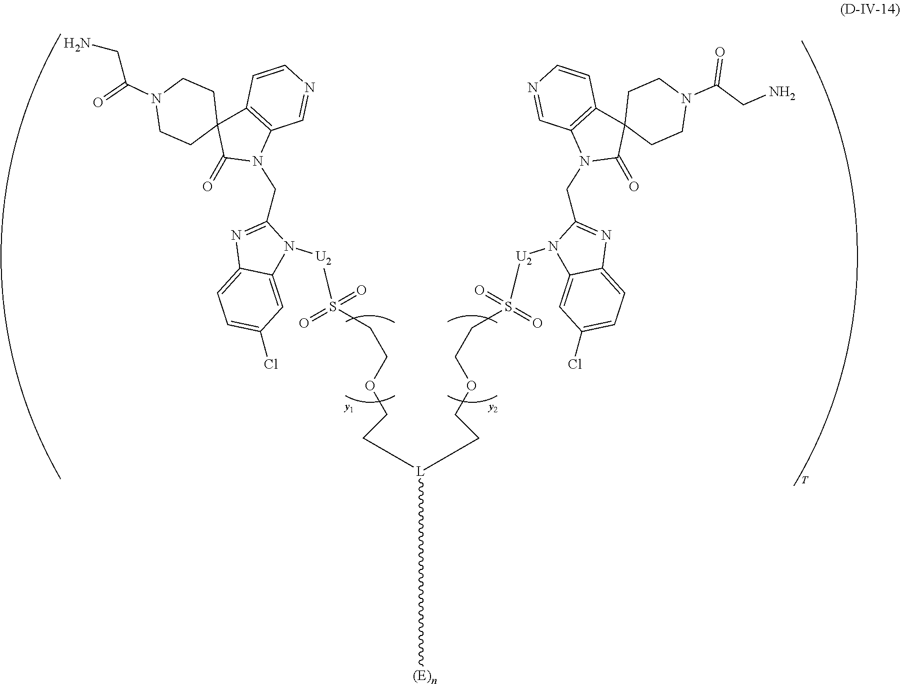

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In certain embodiments, the conjugate is described by formula (D-IV-15):

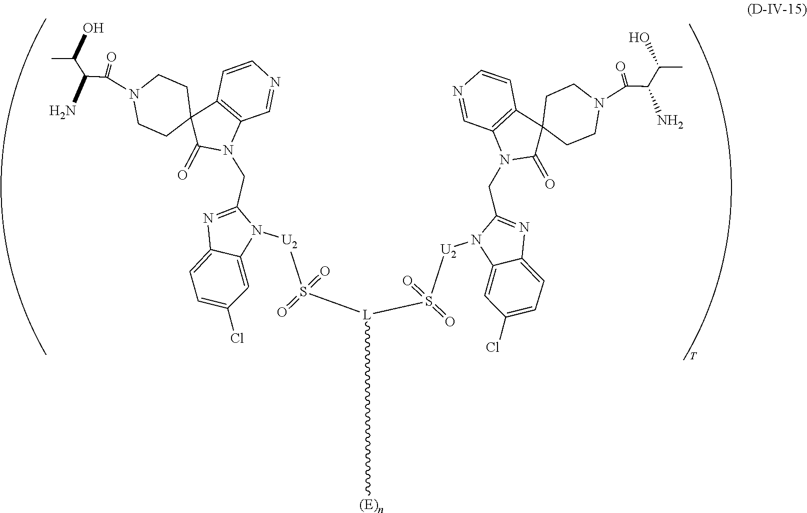

or a pharmaceutically acceptable salt thereof.

In some embodiments, the conjugate is described by formula (D-IV-16):

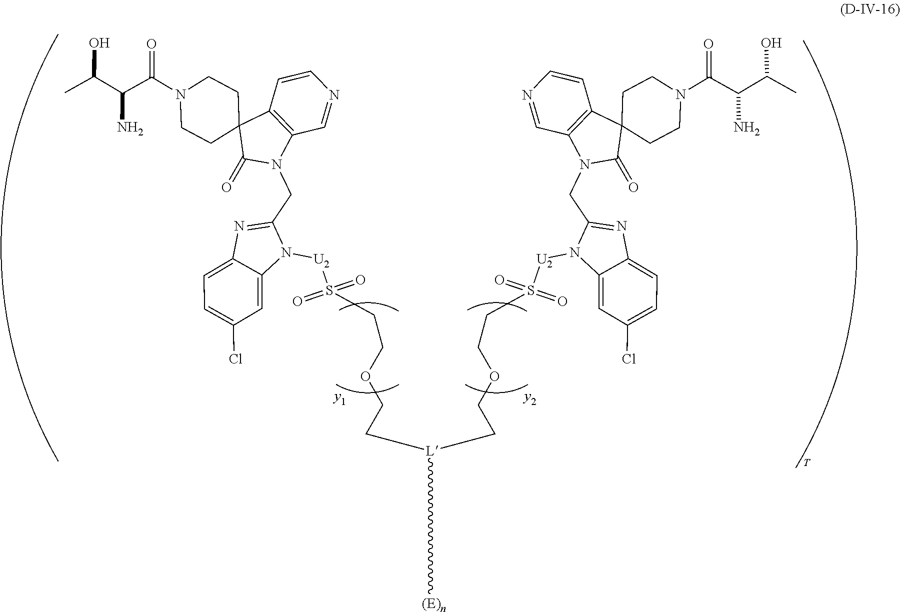

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-IV-17):

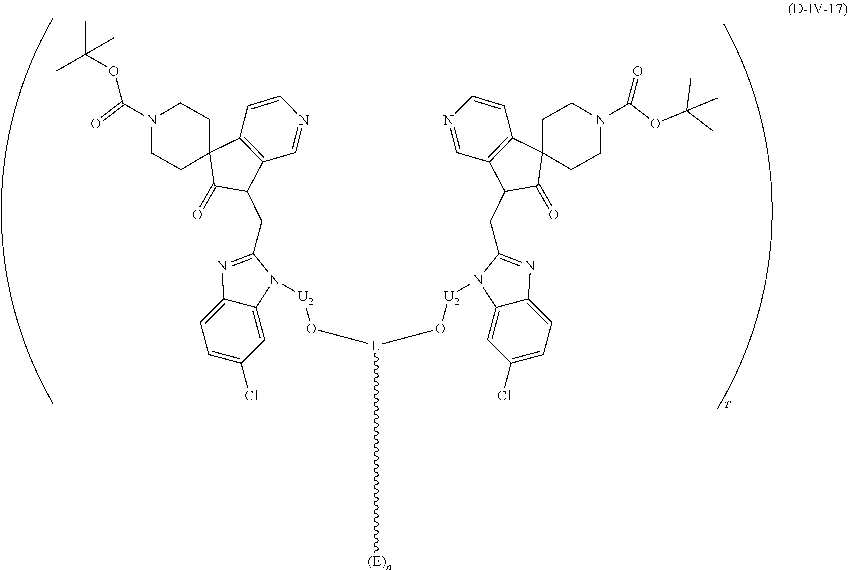

or a pharmaceutically acceptable salt thereof.

In certain embodiments, the conjugate is described by formula (D-IV-18):

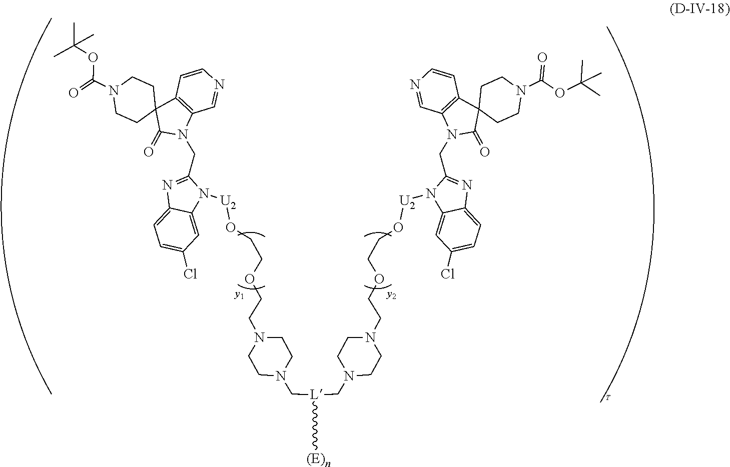

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments of any of the aspects described herein, L or L′ includes one or more optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene, optionally substituted C₂-C₁₅ heteroarylene, O, S, NRⁱ, P, carbonyl, thiocarbonyl, sulfonyl, phosphate, phosphoryl, or imino, wherein Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₂-C₁₅ heteroaryl.

In some embodiments of any of the aspects described herein, the backbone of L or L′ consists of one or more optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene, optionally substituted C₂-C₁₅ heteroarylene, O, S, NRⁱ, P, carbonyl, thiocarbonyl, sulfonyl, phosphate, phosphoryl, or imino, wherein Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₂-C₁₅ heteroaryl.

In some embodiments of any of the aspects described herein, L or L′ is oxo substituted. In some embodiments, the backbone of L or L′ includes no more than 250 atoms. In some embodiments, L or L′ is capable of forming an amide, a carbamate, a sulfonyl, or a urea linkage. In some embodiments L or L′ is a bond. In some embodiments, L or L′ is an atom.

In some embodiments of any of the aspects described herein, each L is described by formula (D-L-I):

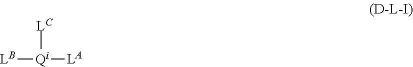

wherein L^{A} is described by formula G^{A1}-(Z^{A1})_{g1}—(Y^{A1})ₕ₁—(Z^{A2})ᵢ₁—(Y^{A2})ⱼ₁—(Z^{A3})ₖ₁—(Y^{A3})ₗ₁—(Z^{A4})ₘ₁—(Y^{A4})ₙ₁—(Z^{A5})_{O1}-G^{A2}; L^{B} is described by formula G^{B1}-(Z^{B1})_{g2}—(Y^{B1})ₕ₂—(Z^{B2})ᵢ₂—(Y^{B2})ⱼ₂—(Z^{B3})ₖ₂—(Y^{B3})ₗ₂—(Z^{B4})ₘ₂—(Y^{B4})ₙ₂—(Z^{B5})_{O2}-G^{B2}; L^{C} is described by formula G^{C1}-(Z^{C1})_{g3}—(Y^{C1})ₕ₃—(Z^{C2})ᵢ₃—(Y^{C2})ⱼ₃—(Z^{C3})ₖ₃—(Y^{C3})ₗ₃—(Z^{C4})ₘ₃—(Y^{C4})ₙ₃—(Z^{C5})_{O3}-G^{C2}; G^{A1} is a bond attached to Qⁱ; G^{A2} is a bond attached to A1; G^{B1} is a bond attached to Qⁱ; G^{B2} is a bond attached to A2; G^{C1} is a bond attached to Qⁱ; G^{C2} is a bond attached to E or a functional group capable of reacting with a functional group conjugated to E (e.g., maleimide and cysteine, amine and activated carboxylic acid, thiol and maleimide, activated sulfonic acid and amine, isocyanate and amine, azide and alkyne, and alkene and tetrazine); each of Z^{A1}, Z^{A2}, Z^{A3}, Z^{A4}, Z^{A5}, Z^{B1}, Z^{B2}, Z^{B3}, Z^{B4}, Z^{B5}, Z^{C1}, Z^{C2}, Z^{C3}, Z^{C4}, and Z^{C5} is, independently, optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene, or optionally substituted C₂-C₁₅ heteroarylene; each of Y^{A1}, Y^{A2}, Y^{A3}, Y^{A4}, Y^{B1}, Y^{B2}, Y^{B3}, Y^{B4}, Y^{C1}, Y^{C2}, Y^{C3}, and Y^{C4} is, independently, O, S, NRⁱ, P, carbonyl, thiocarbonyl, sulfonyl, phosphate, phosphoryl, or imino; Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₂-C₁₅ heteroaryl; each of g1, h1, i1, j1, k1, l1, m1, n1, o1, g2, h2, i2, j2, k2, l2, m2, n2, o2, g3, h3, i3, j3, k3, l3, m3, n3, and o3 is, independently, 0 or 1; Qⁱ is a nitrogen atom, optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene, or optionally substituted C₂-C₁₅ heteroarylene.

In some embodiments, optionally substituted includes substitution with a polyethylene glycol (PEG). A PEG has a repeating unit structure (—CH₂CH₂O—)ₙ, wherein n is an integer from 2 to 100. A polyethylene glycol may selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀).

In some embodiments, L^{C} may have two points of attachment to the Fc domain, Fc-binding peptide, albumin protein, or albumin protein-binding peptide (e.g., two G^{C2}).

In some embodiments of any of the aspects described herein, L includes a polyethylene glycol (PEG) linker. A PEG linker includes a linker having the repeating unit structure (—CH₂CH₂O—)ₙ, wherein n is an integer from 2 to 100. A polyethylene glycol linker may covalently join a RSV F protein inhibitor and E (e.g., in a conjugate of any one of formulas (M-I)-(M-IV)). A polyethylene glycol linker may covalently join a first RSV F protein inhibitor and a second RSV F protein inhibitor (e.g., in a conjugate of any one of formulas (D-I)-(D-IV)). A polyethylene glycol linker may covalently join an RSV F protein inhibitor dimer and E (e.g., in a conjugate of any one of formulas (D-I)-(D-IV)). A polyethylene glycol linker may selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀). In some embodiments, L^{c} includes a PEG linker, where L^{C} is covalently attached to each of Qⁱ and E.

In some embodiments, L is

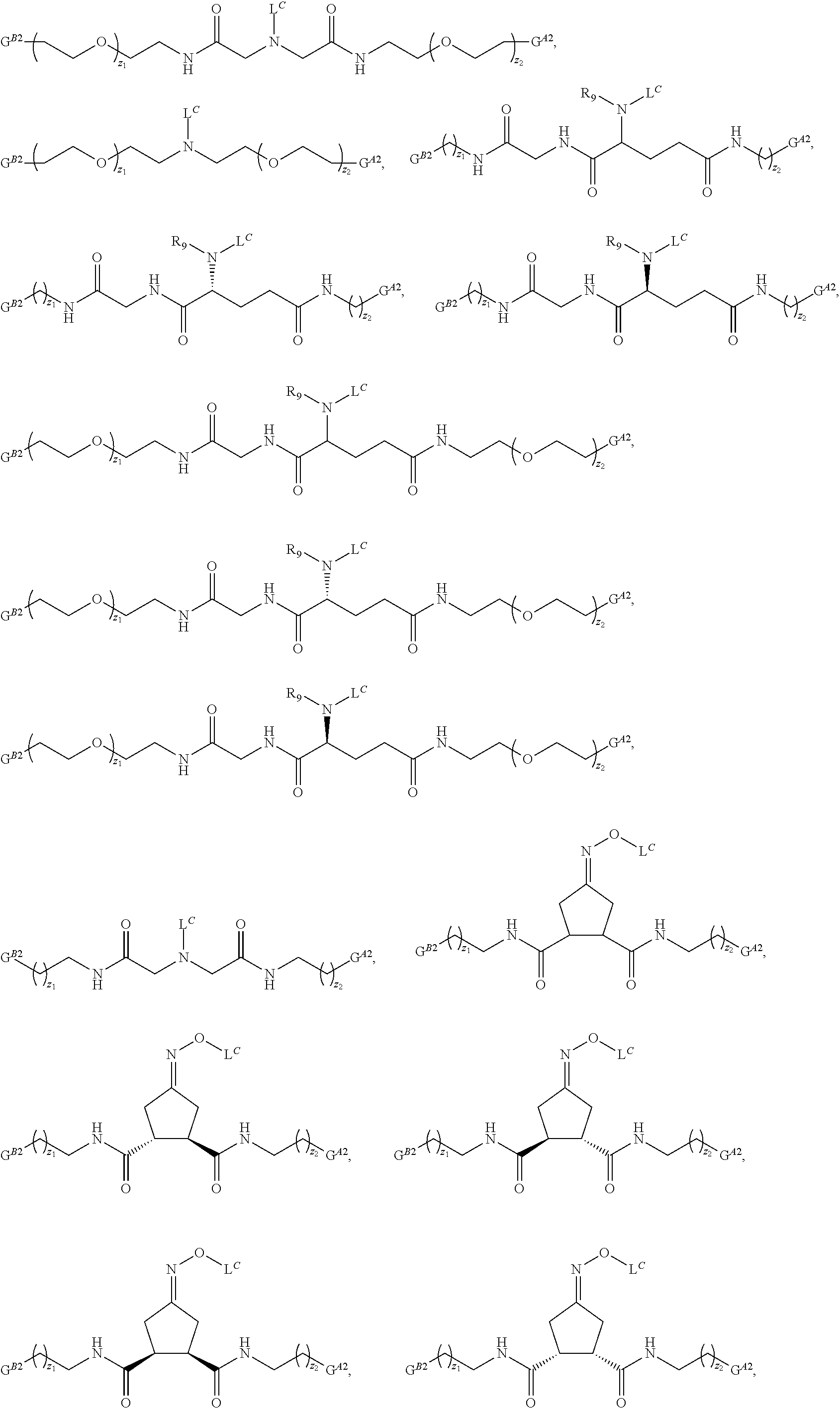

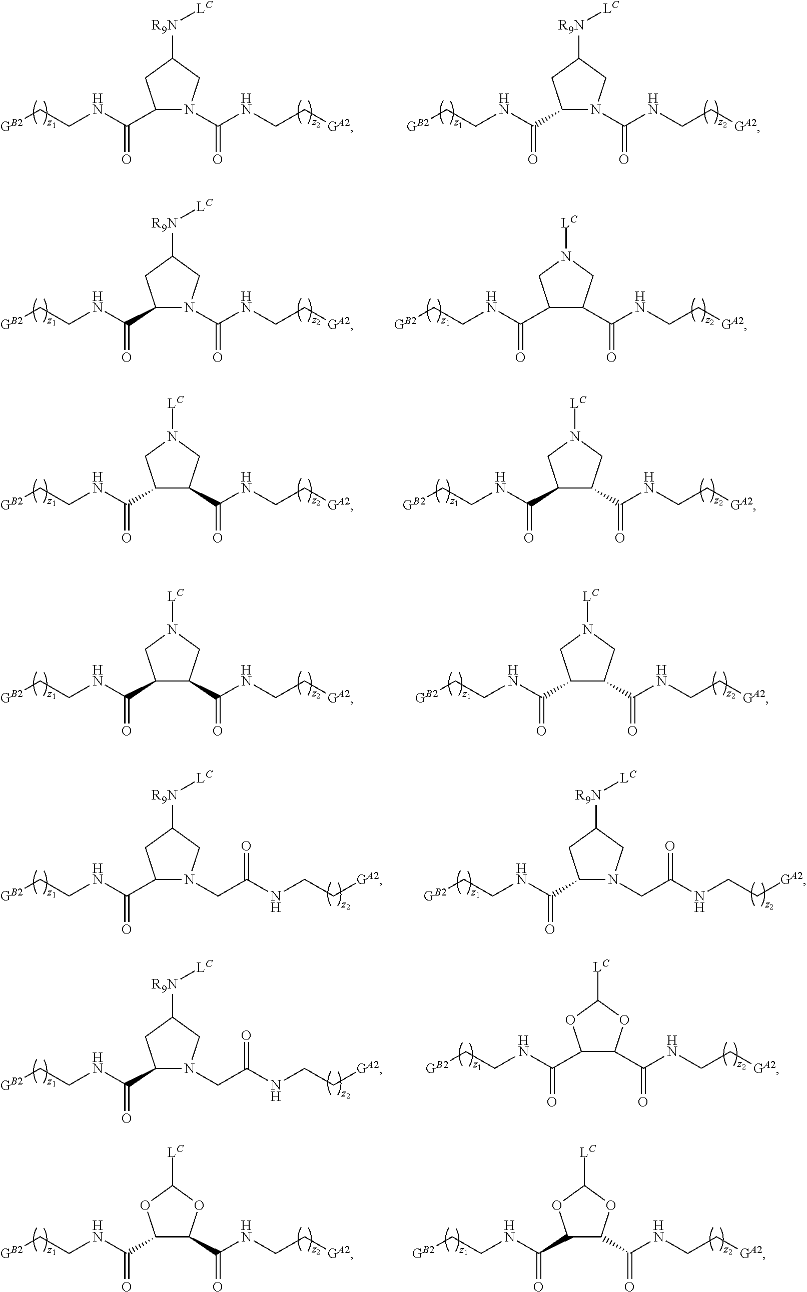

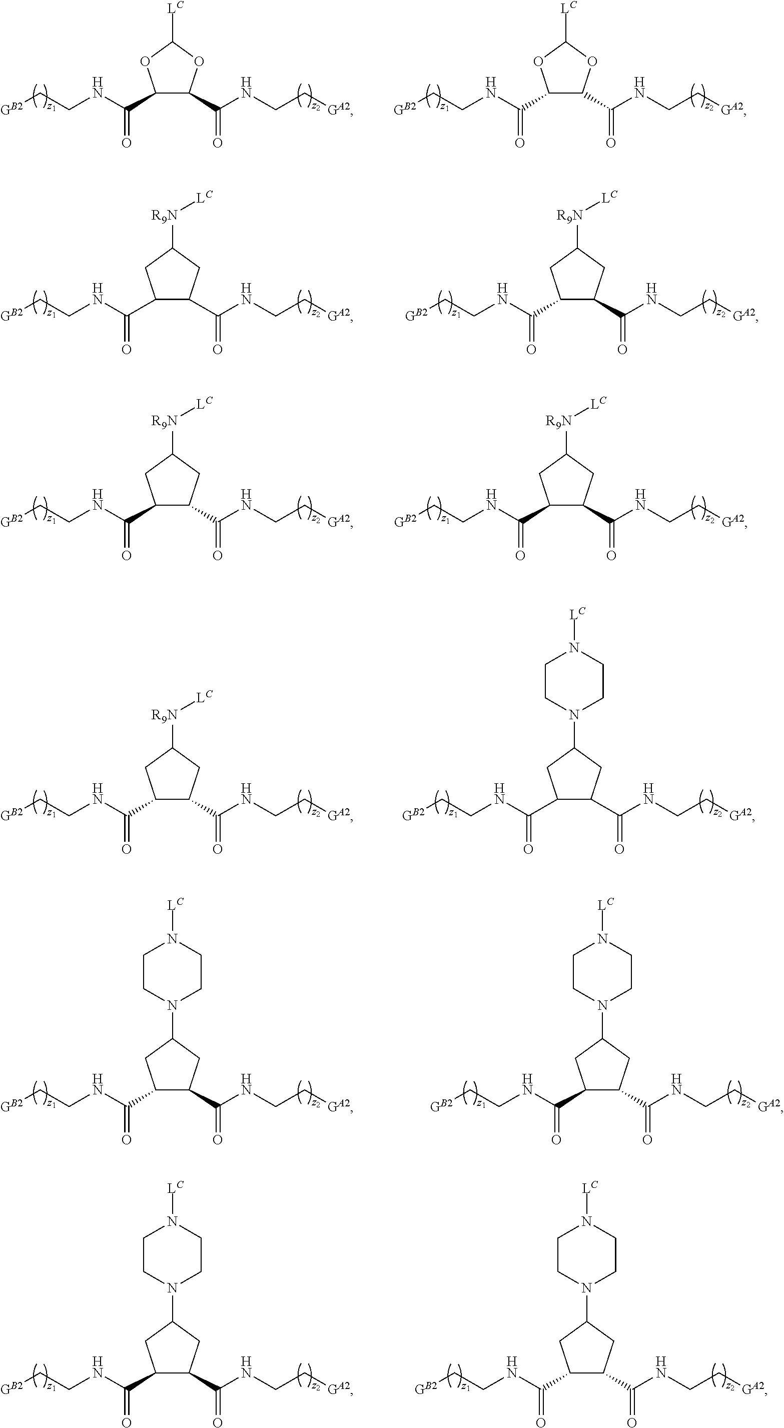

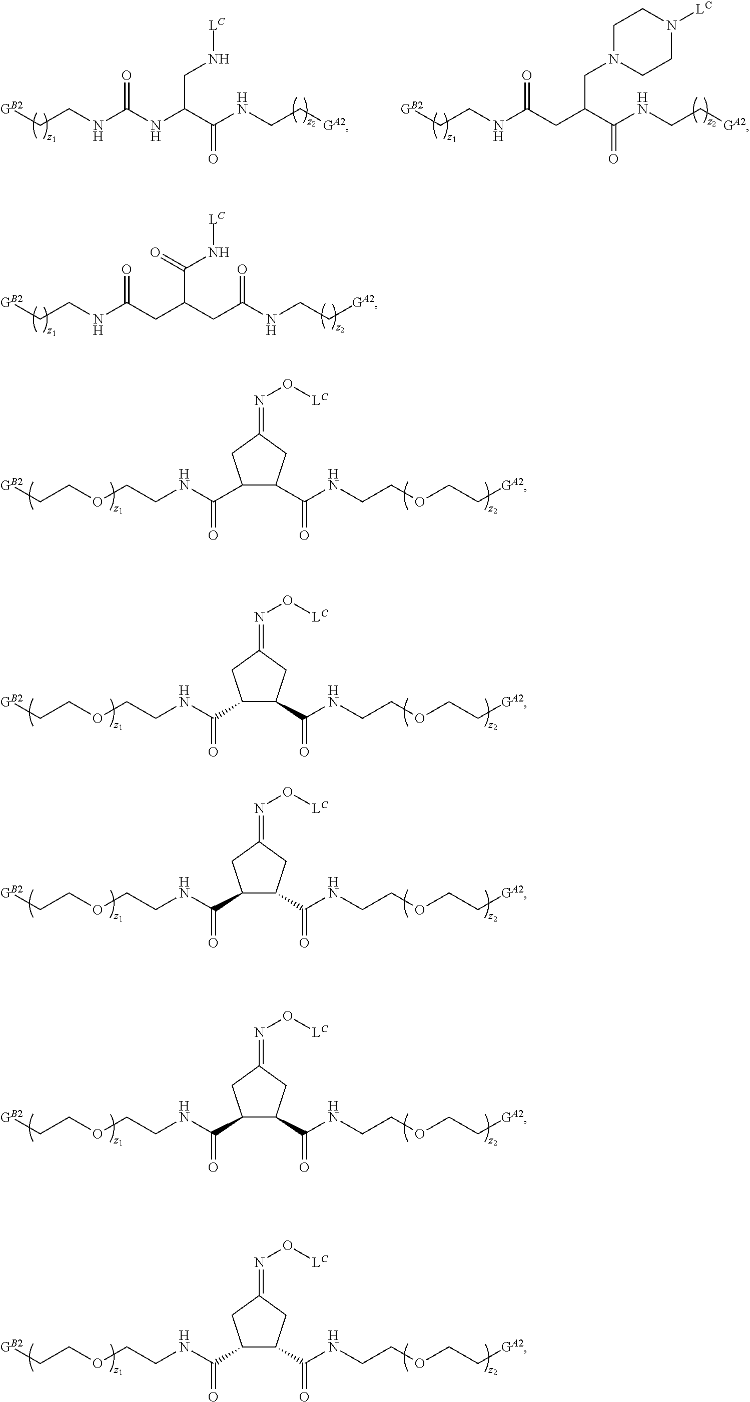

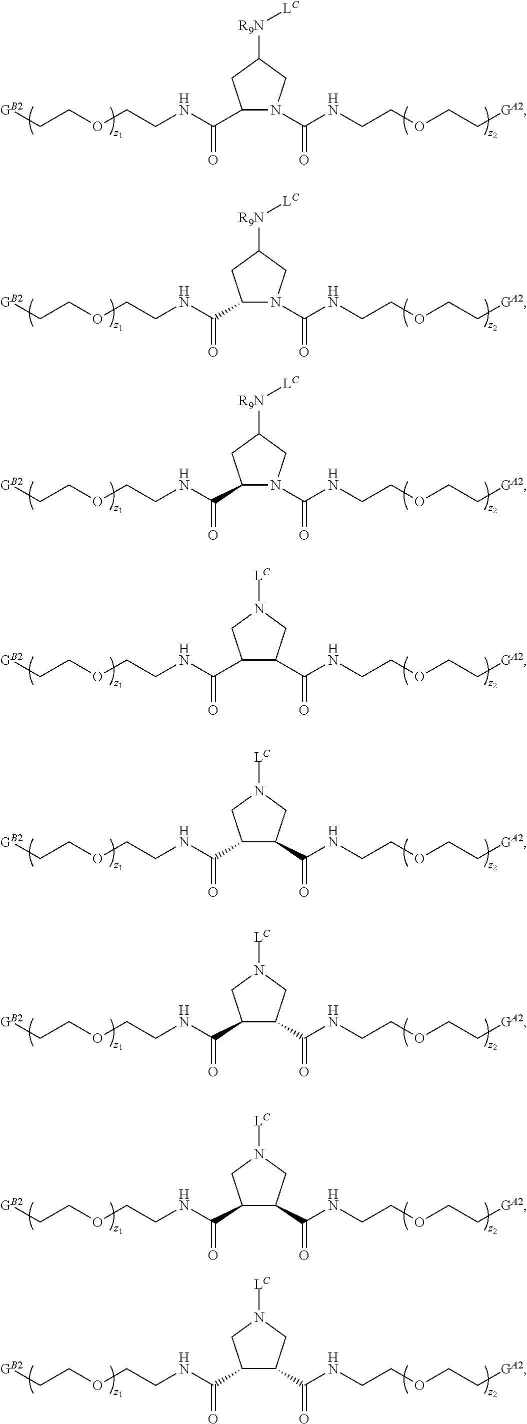

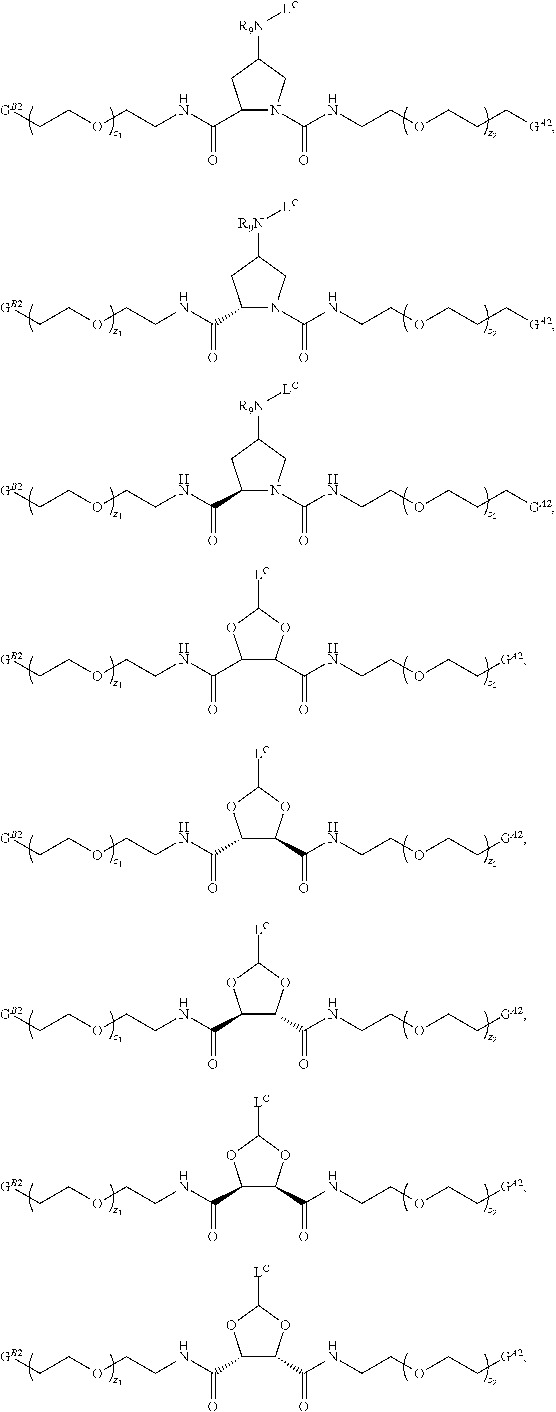

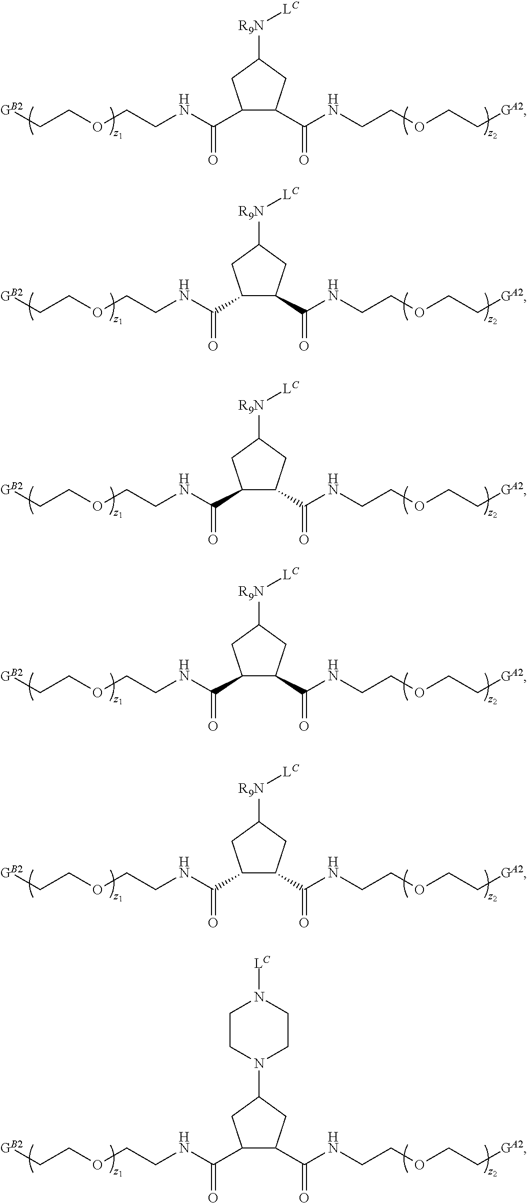

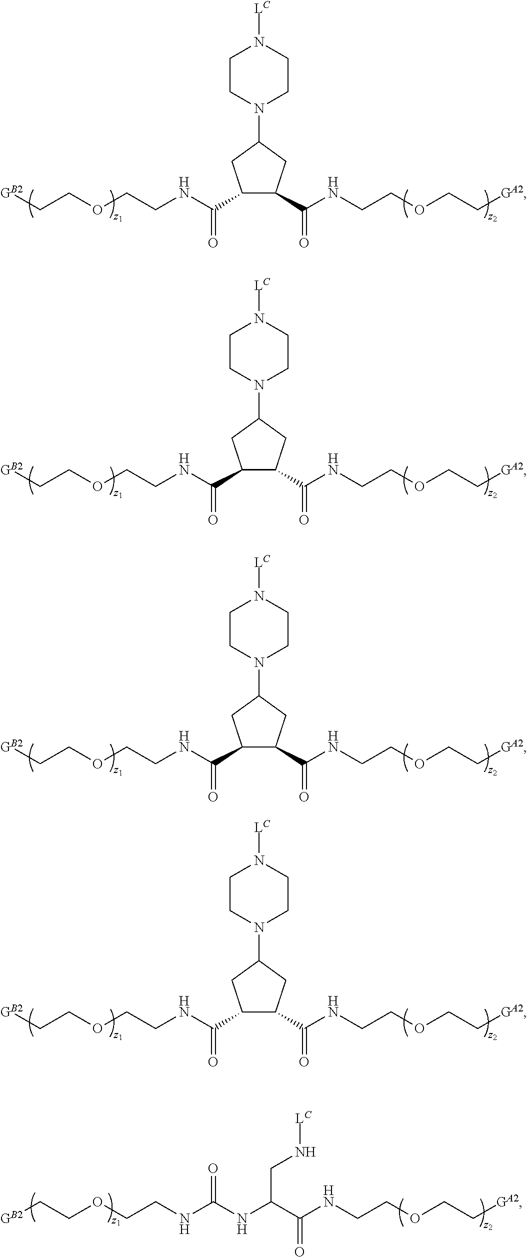

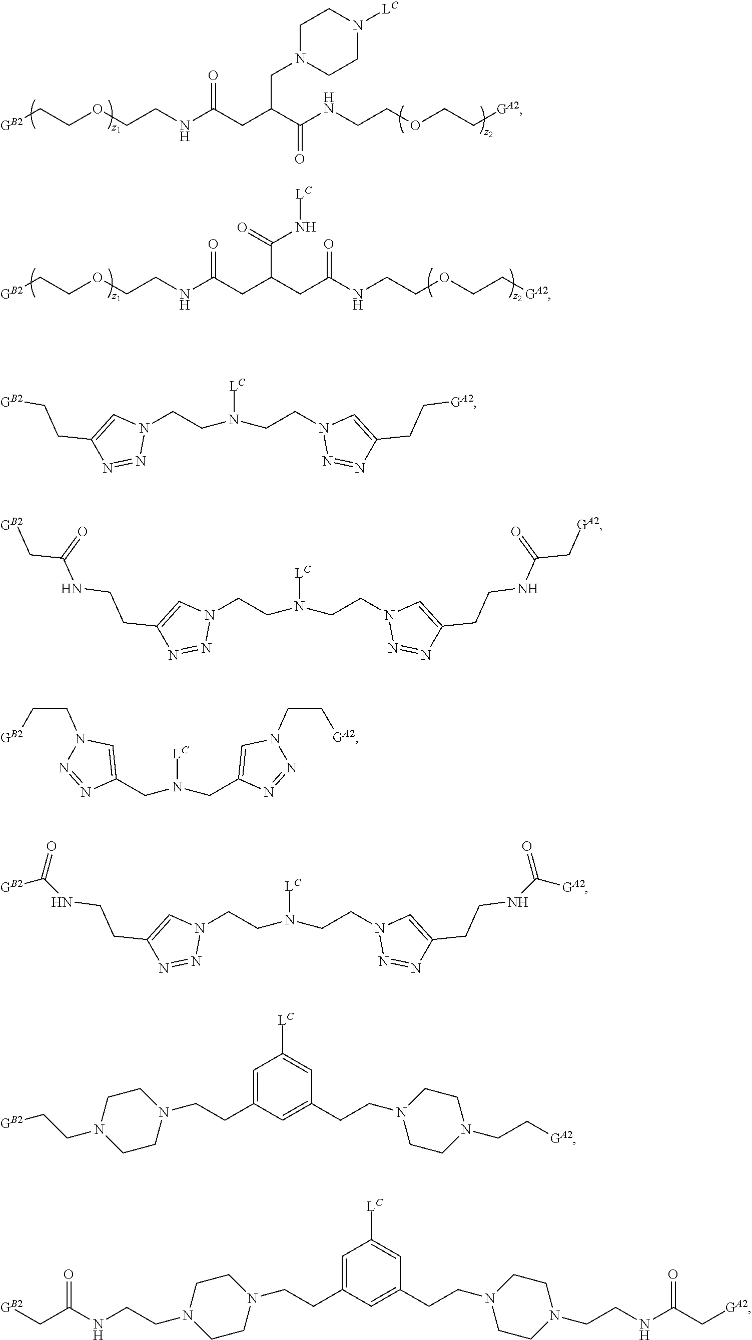

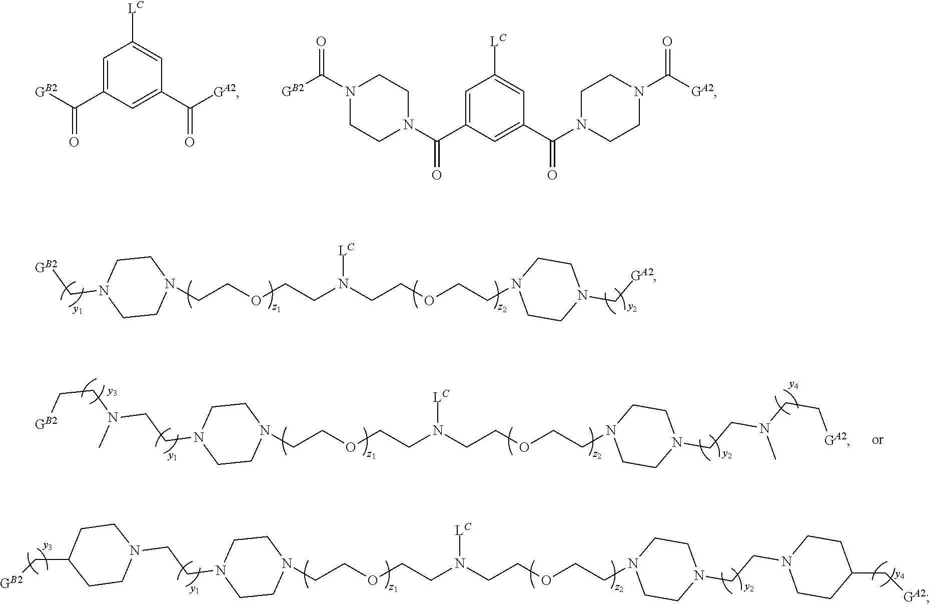

wherein z₁ and z₂ are each, independently, and integer from 1 to 20; and R₉ is selected from H, C1-C20 alkyl, C3-C20 cycloalkyl, C₂-C₂₀ heterocycloalkyl; C5-C15 aryl, and C2-C15 heteroaryl.

In some embodiments, L is

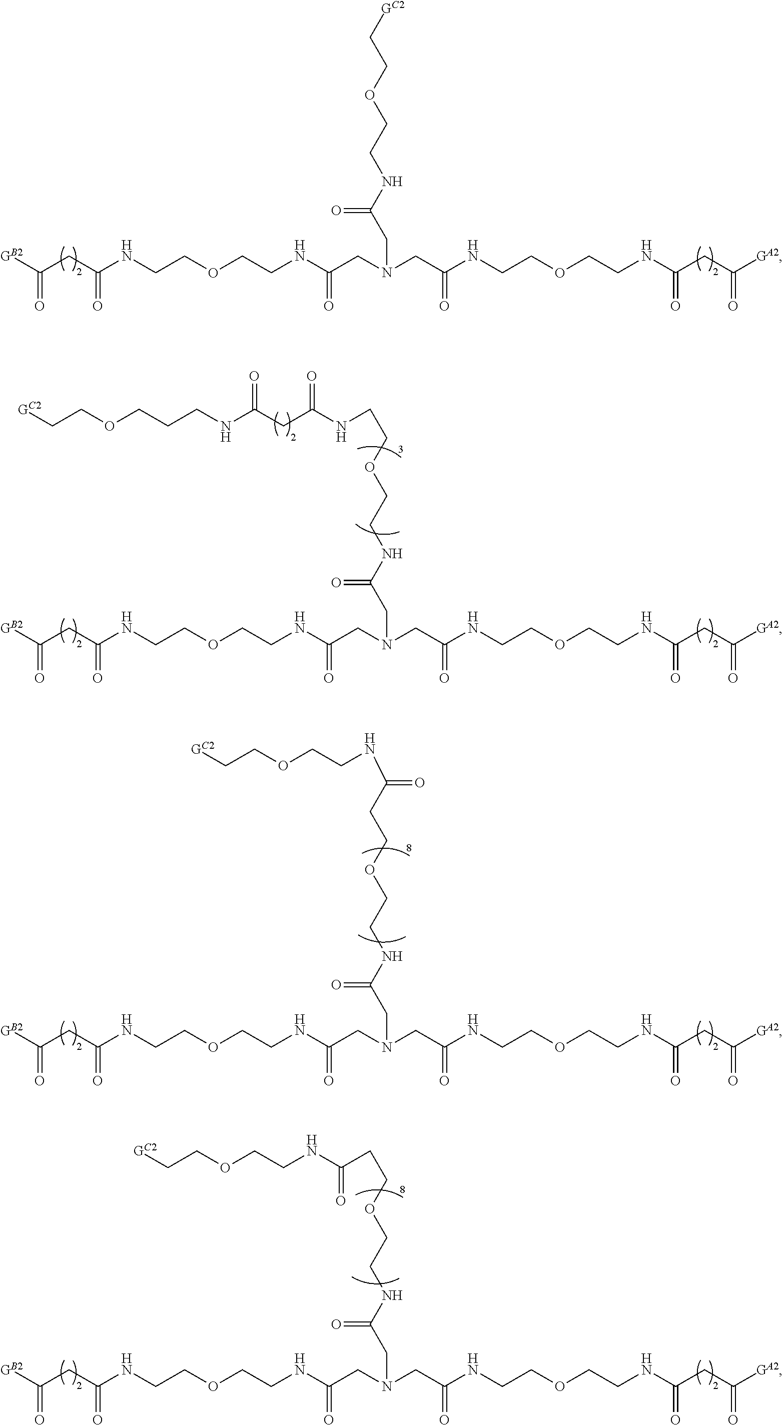

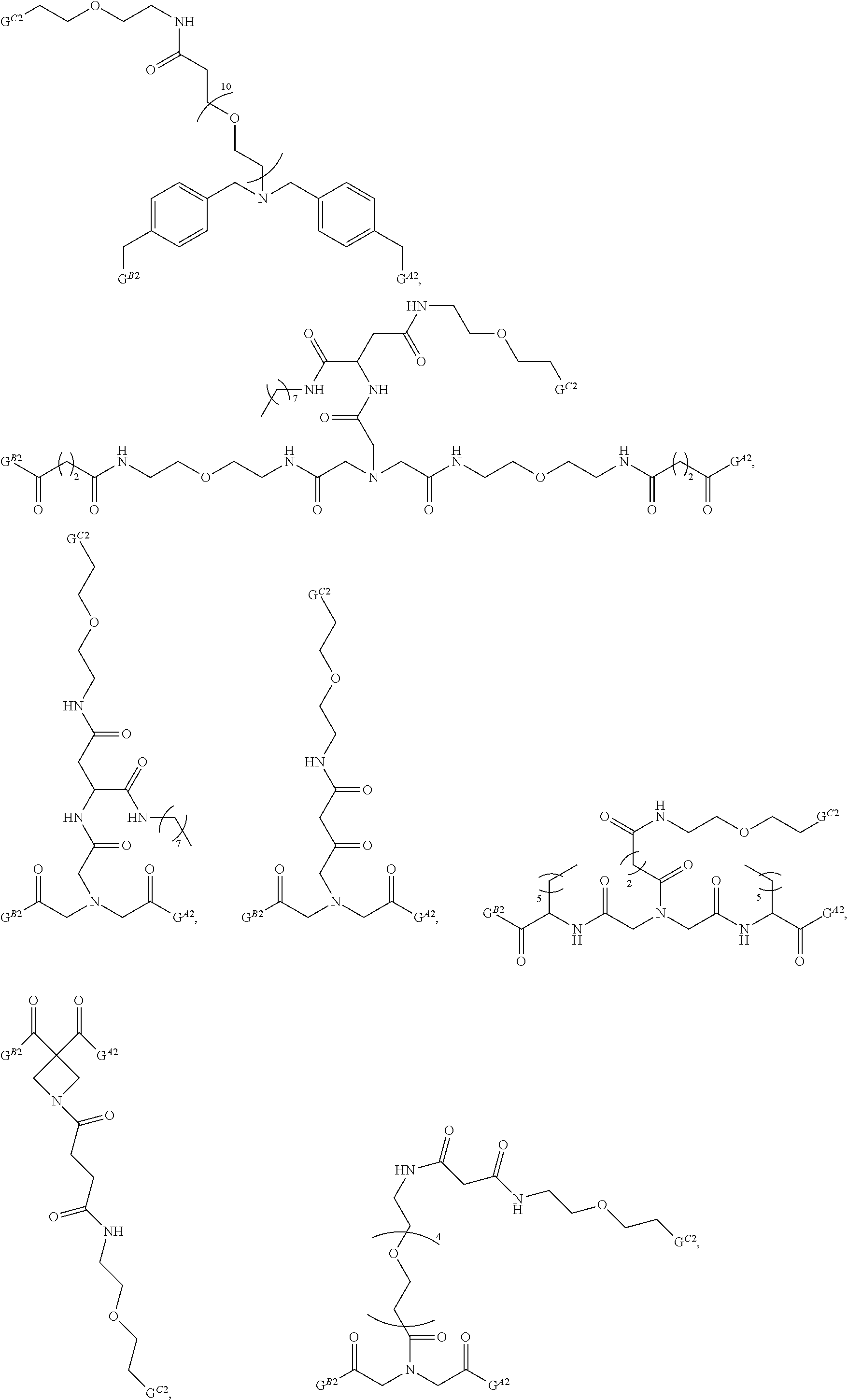

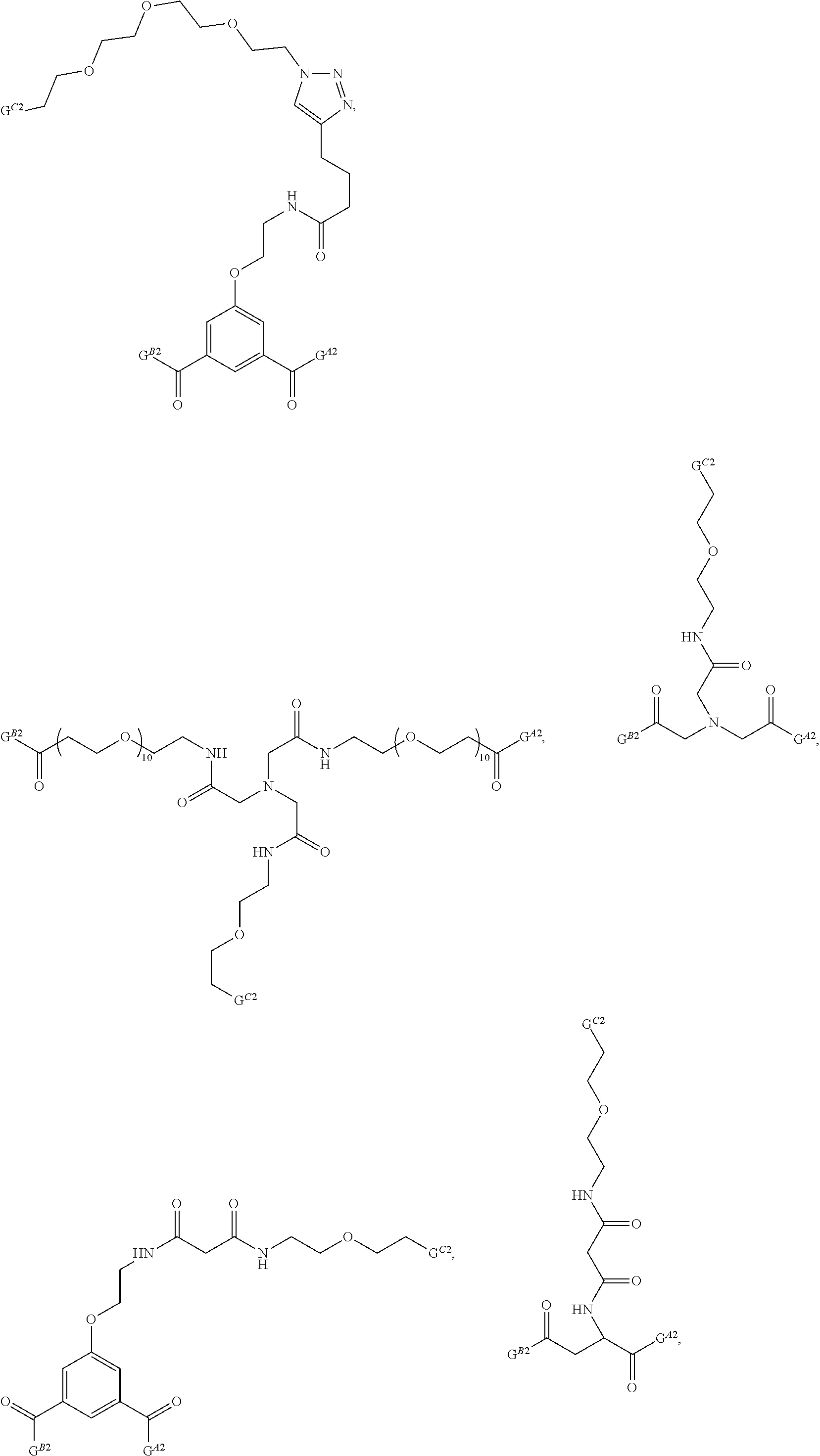

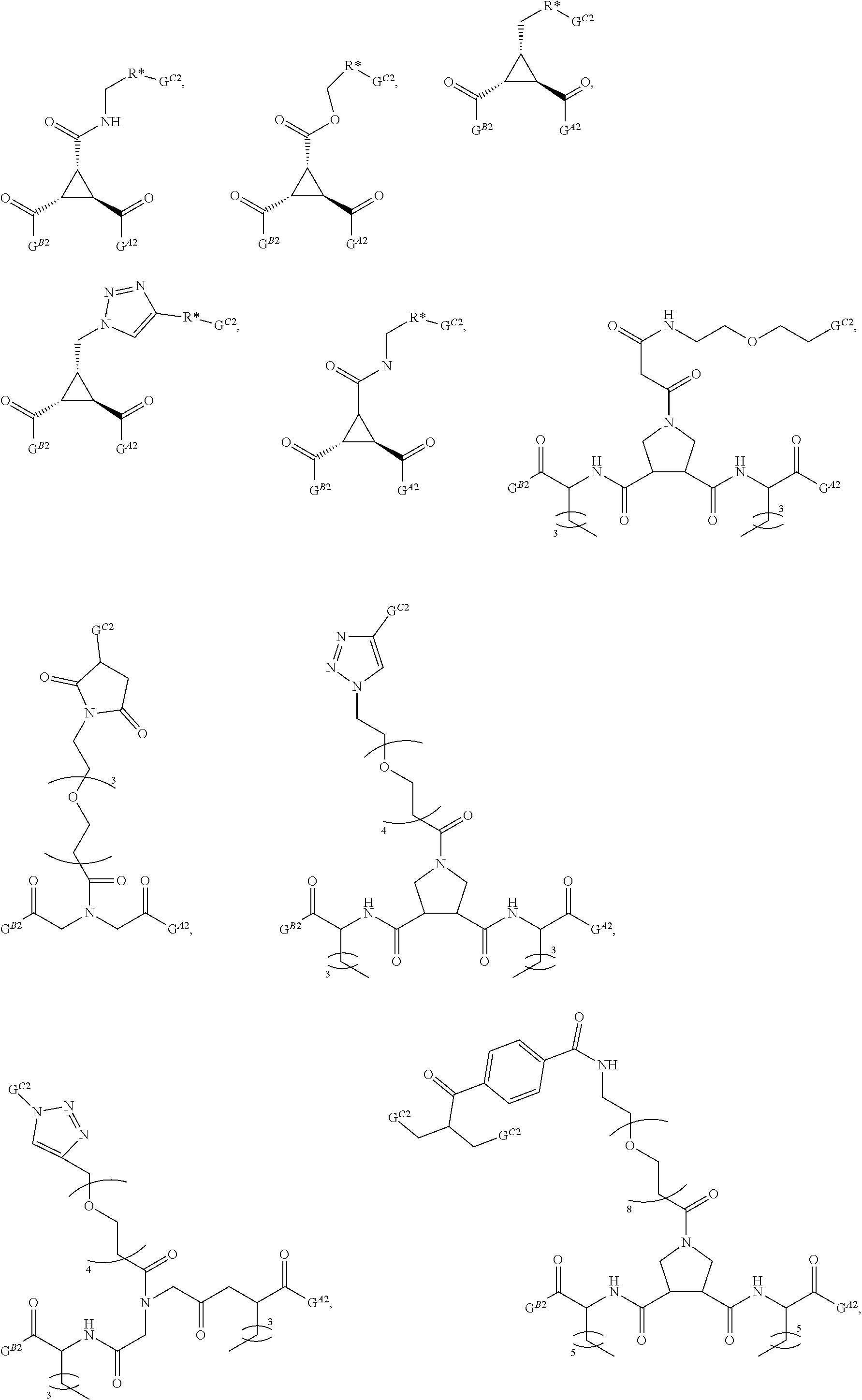

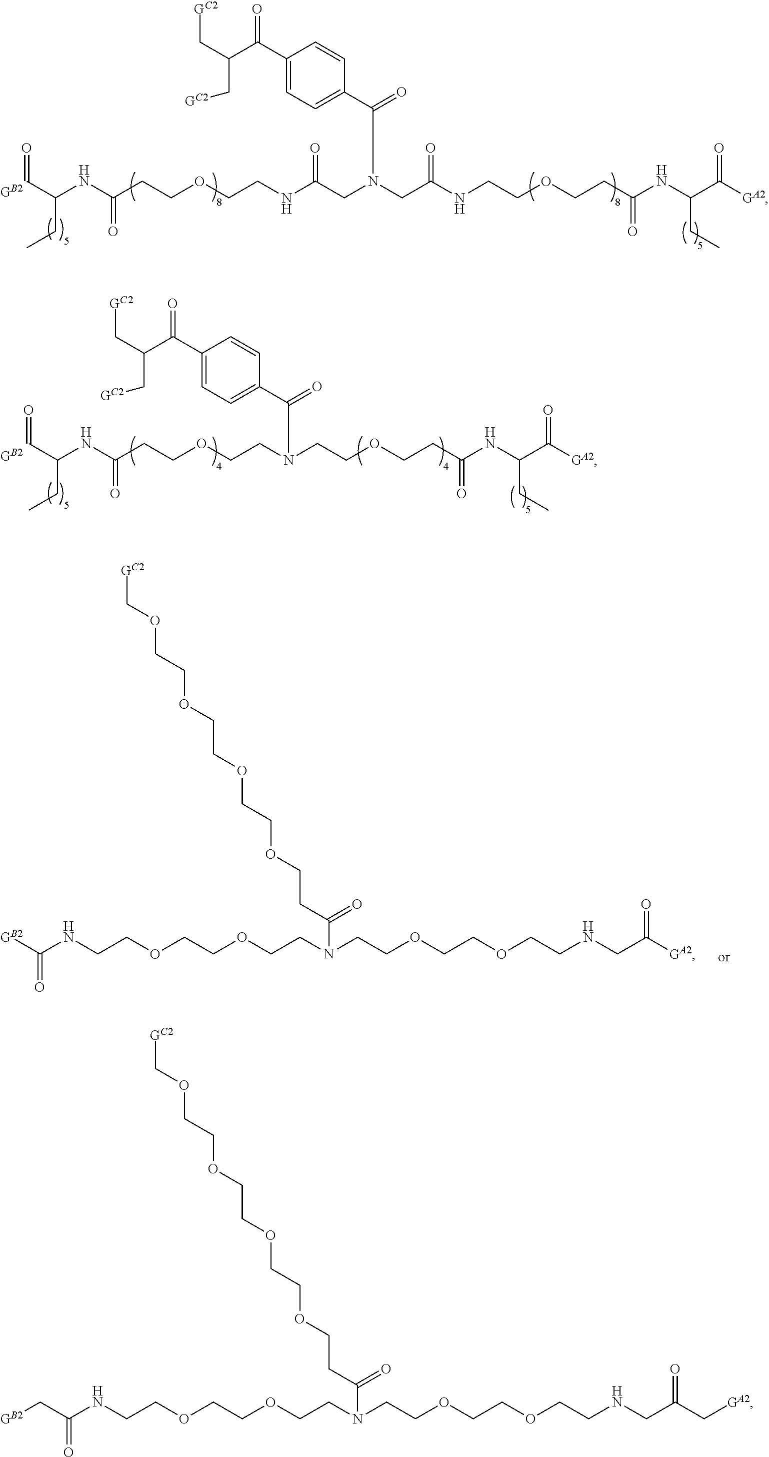

wherein R* is a bond or includes one or more of optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₅-C₂₀ heterocycloalkynylene, optionally substituted C₅-C₁₅ arylene, optionally substituted C₃-C₁₅ heteroarylene, O, S, NRⁱ, P, carbonyl, thiocarbonyl, sulfonyl, phosphate, and imino, and wherein Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₃-C₁₅ heteroaryl.

In another aspect, the invention features a conjugate described by formula (M-I):

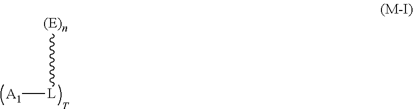

wherein each E independently includes an Fc domain monomer (e.g., an Fc domain monomer having the sequence of any one of SEQ ID NOs: 1-95), an albumin protein (e.g., an albumin protein having the sequence of any one of SEQ ID NOs: 96-98), an albumin protein-binding peptide, or an Fc-binding peptide; n is 1 or 2; T is an integer from 1 to 20 (e.g., T is 1, 2, 3, 4, 5, 6, 7, 8, 9, 10, 11, 12, 13, 14, 15, 16, 17, 18, 19, or 20); and L is a linker covalently attached to each of E and A₁, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each A₁ may be independently selected from a structure described by any one of formulas (A-I)-(A-III).

In some embodiments, the conjugate is described by formula (M-II):

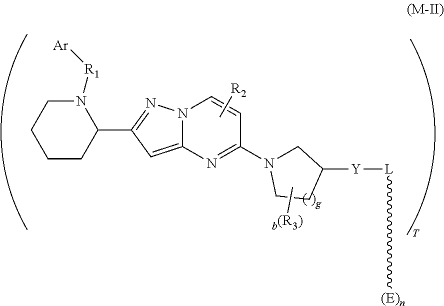

or a pharmaceutically acceptable salt thereof.

In some embodiments, conjugate is described by formula (M-II-1):

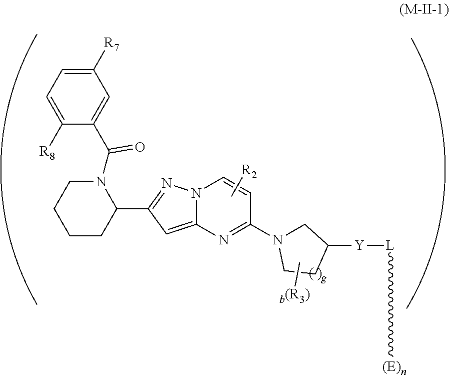

wherein R₇ and R₈ are each independently selected from OH, halogen, nitrile, nitro, optionally substituted amine, optionally substituted imine, optionally substituted C₁-C₂₀ alkamino, optionally substituted sulfhydryl, optionally substituted carboxyl, optionally substituted cyano, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;
or a pharmaceutically acceptable salt thereof.

In some embodiments, the conjugate is described by formula (M-II-2):

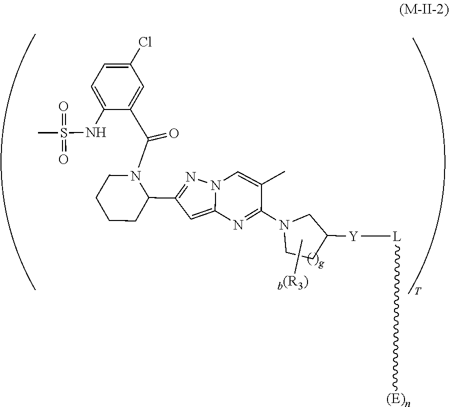

or a pharmaceutically acceptable salt thereof.

In some embodiments, the conjugate is described by the formula (M-II-3)

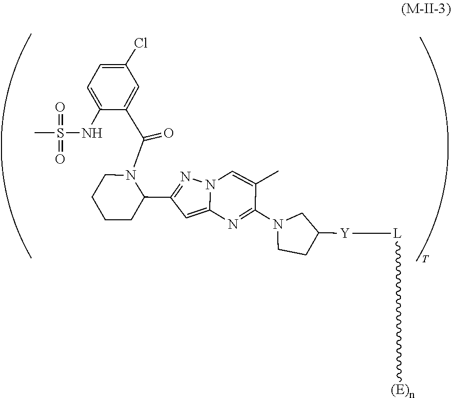

or a pharmaceutically acceptable salt thereof.

In some embodiments, the conjugate is described by the formula (M-II-4):

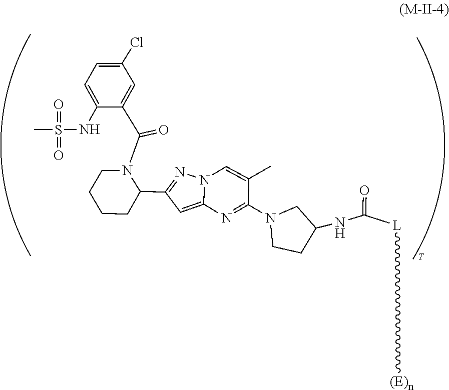

or a pharmaceutically acceptable salt thereof.

In some embodiments, the conjugate is described by the formula (M-II-5):

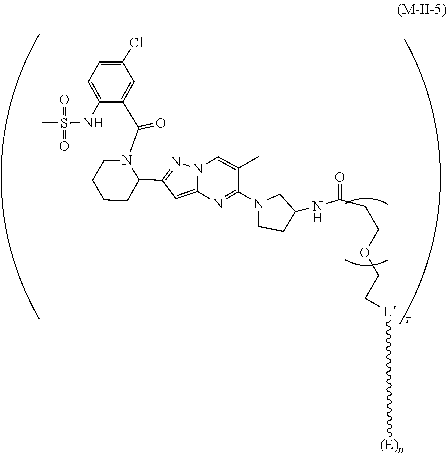

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-II-6):

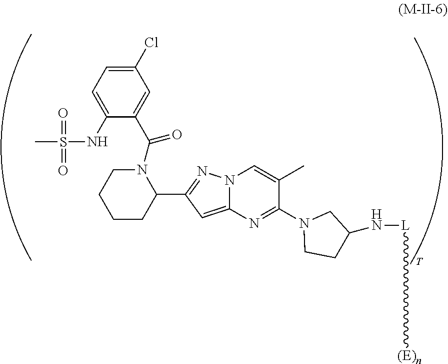

or a pharmaceutically acceptable salt thereof.

In some embodiments, the conjugate is described by the formula (M-II-7):

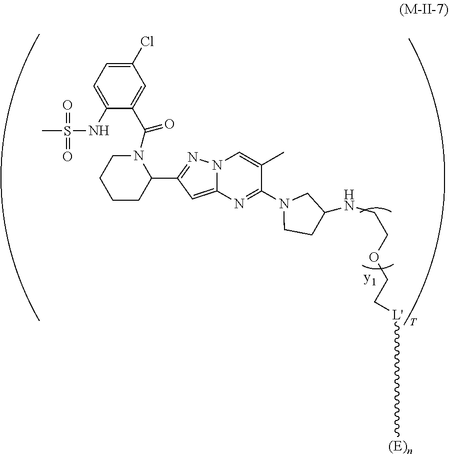

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-II-8):

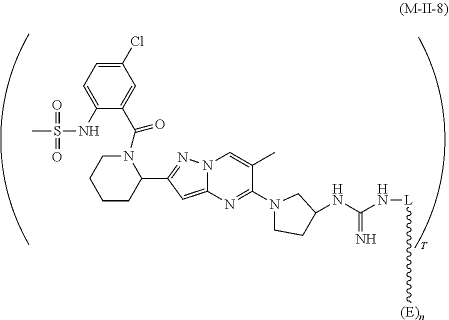

or a pharmaceutically acceptable salt thereof.

In some embodiments, the conjugate is described by formula (M-II-9):

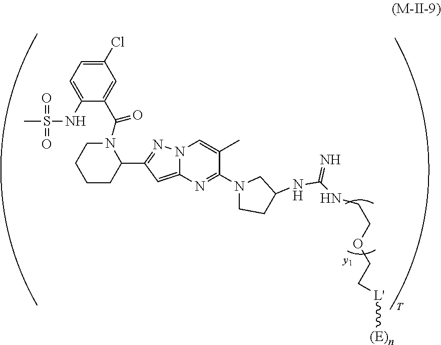

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, conjugate is described by the formula (M-II-10):

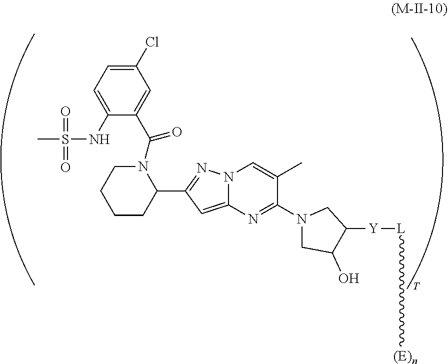

or a pharmaceutical acceptable salt thereof.

In some embodiments, the conjugate is described by formula (M-II-11):

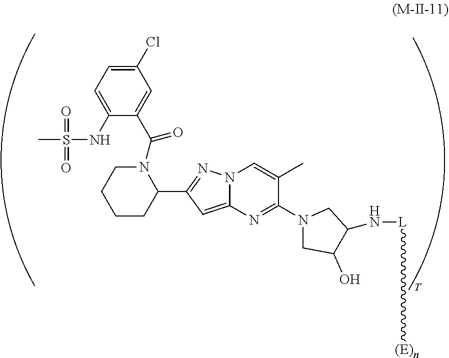

or a pharmaceutically acceptable salt thereof.

In some embodiments, the conjugate is described by the formula (M-II-12):

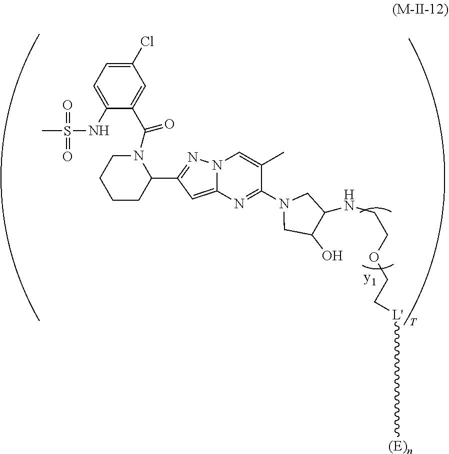

wherein L′ is the remainder of L, and y₁ is an integer from 1-20 (e.g., y₁ is independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-II-13):

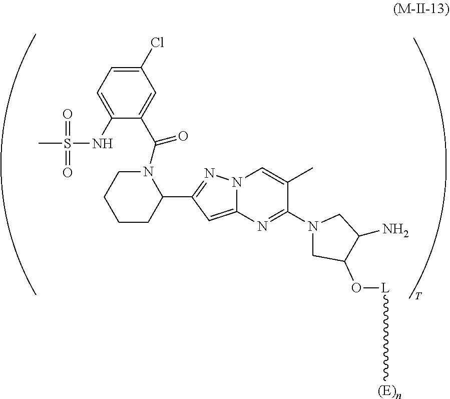

or a pharmaceutically acceptable salt thereof.

In some embodiments, the conjugate is described by formula (M-II-14):

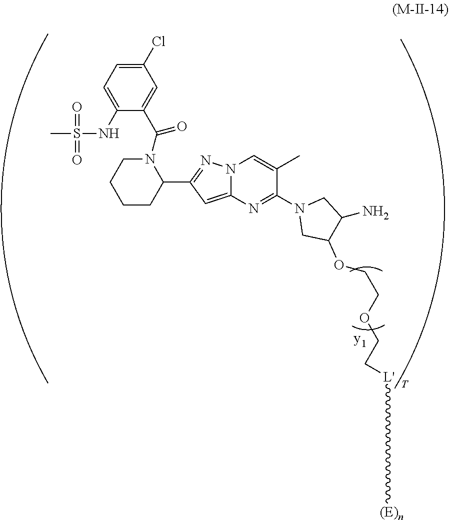

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-II-15):

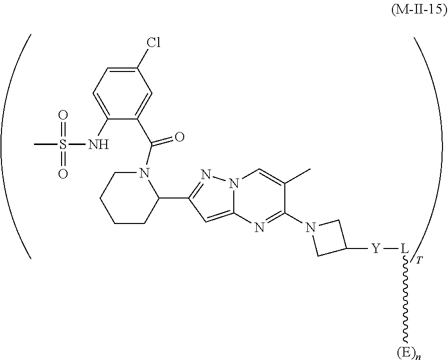

or a pharmaceutically acceptable salt thereof.

In some embodiments, the conjugate is described by formula (M-II-16):

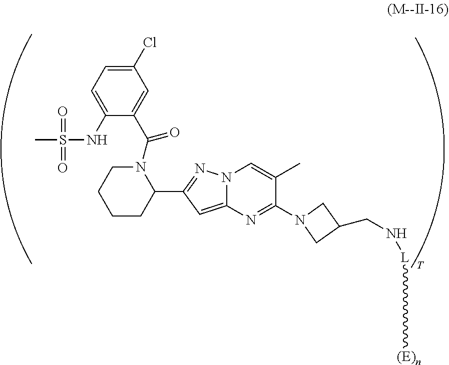

or a pharmaceutically acceptable salt thereof.

In some embodiments, the conjugate is described by formula (M-II-17):

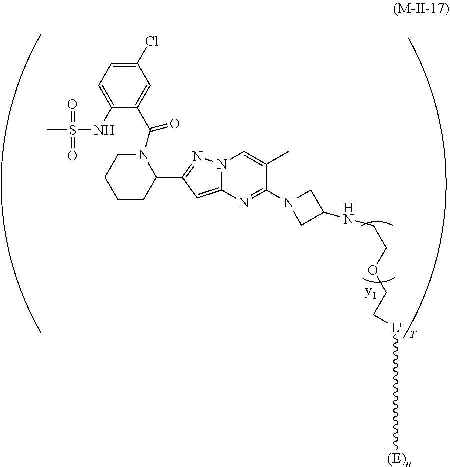

wherein L′ is the remainder of L, and y₁ is an integer from 1-20 (e.g., y₁ is independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-III):

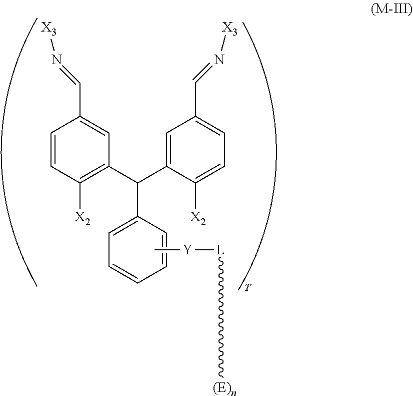

or a pharmaceutically acceptable salt thereof.

In some embodiments, the conjugate is described by formula (M-III-1):

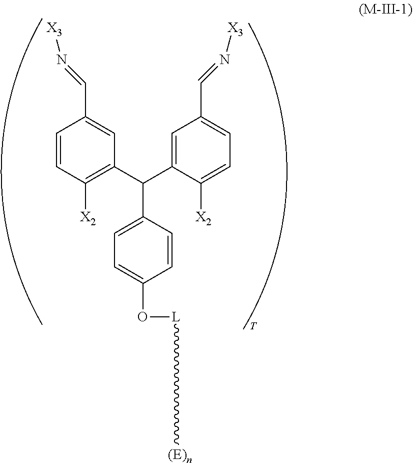

or a pharmaceutically acceptable salt thereof.

In some embodiments, the conjugate is described by formula (M-III-2):

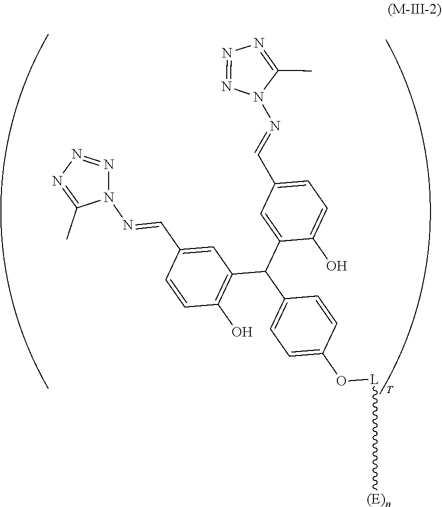

or a pharmaceutically acceptable salt thereof.

In some embodiments, the conjugate is described by formula (M-III-3):

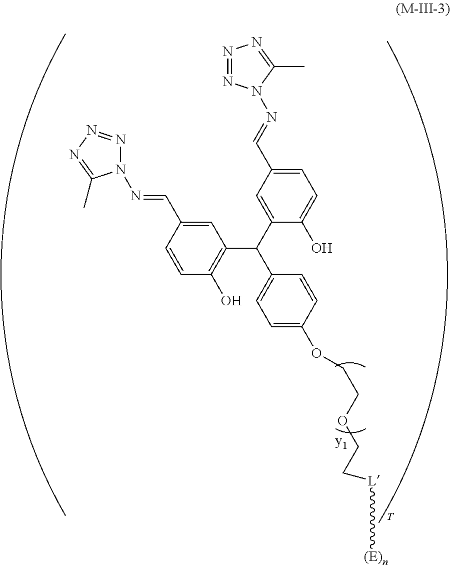

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-IV):

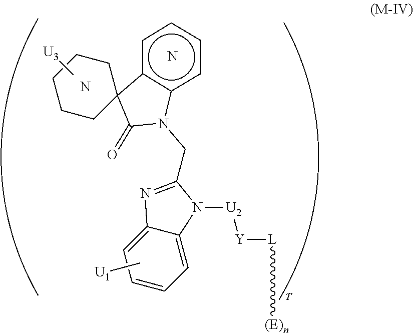

or a pharmaceutically acceptable salt thereof. In preferred embodiments, U₂ is an optionally substituted C₁-C₆ alkyl (e.g., C₁, C₂, C₃, C₄, C₅, or C₆ alkyl),

In some embodiments, the conjugate is described by formula (M-IV-1):

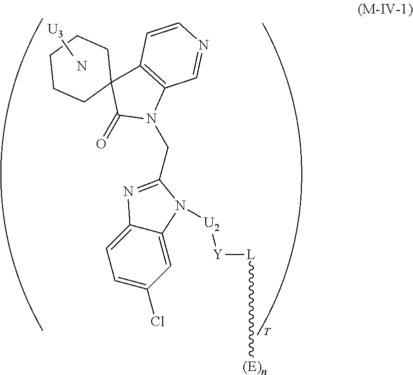

or a pharmaceutically acceptable salt thereof.

In some embodiments, the conjugate is described by formula (M-IV-2):

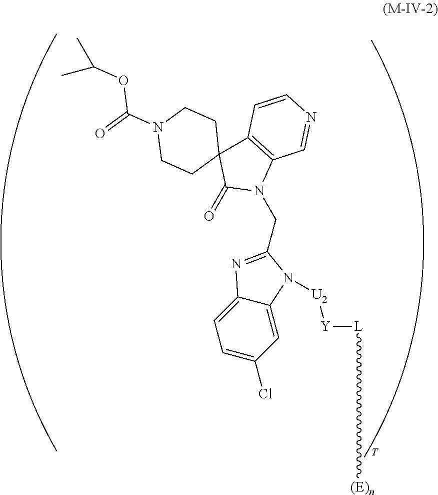

or a pharmaceutically acceptable salt thereof.

In some embodiments, the conjugate is described by formula (M-IV-3):

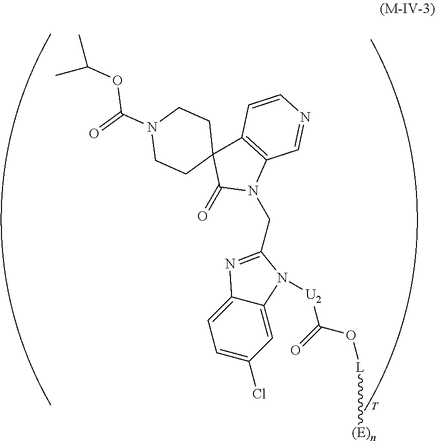

or a pharmaceutically acceptable salt thereof.

In some embodiments, the conjugate is described by formula (M-IV-4):

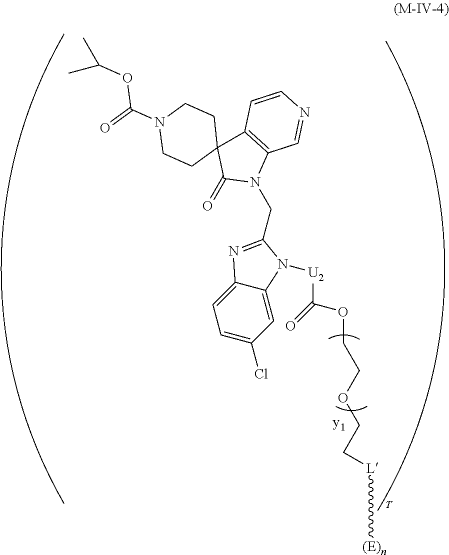

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-IV-5):

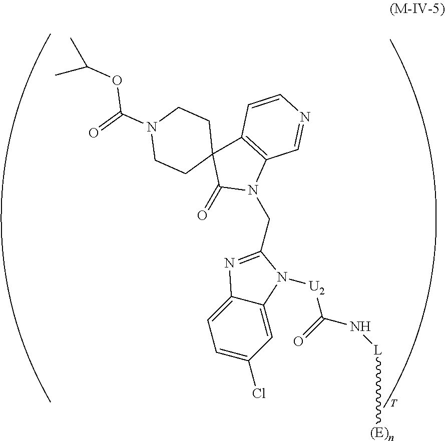

or a pharmaceutically acceptable salt thereof.

In some embodiments, the conjugate is described by formula (M-IV-6):

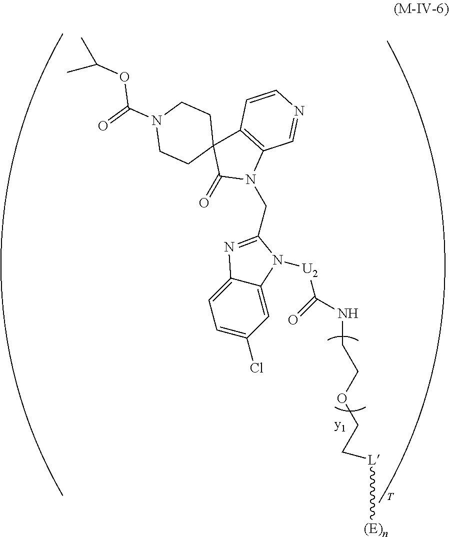

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-IV-7):

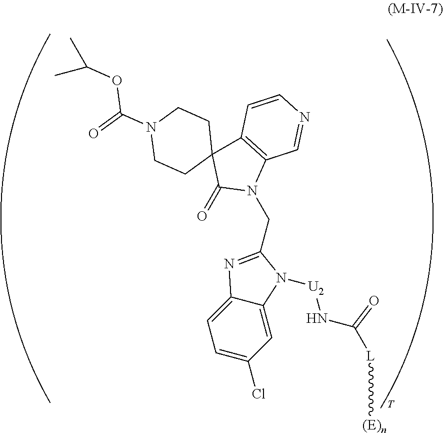

or a pharmaceutically acceptable salt thereof.

In some embodiments, the conjugate is described by formula (M-IV-8):

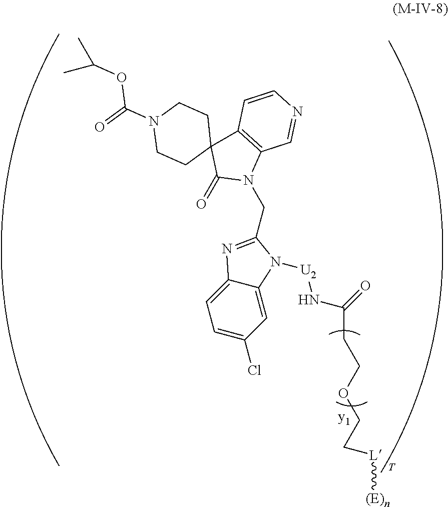

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In certain embodiments, the conjugate is described by formula (M-IV-9):

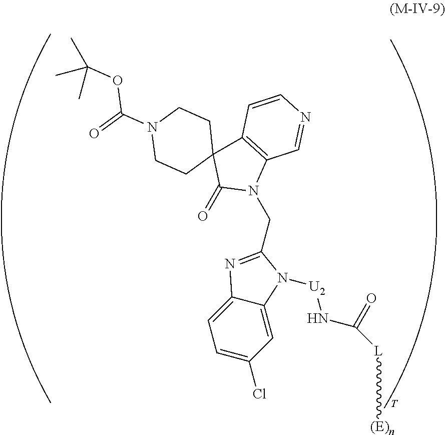

or a pharmaceutically acceptable salt thereof.

In some embodiments, the conjugate is described by formula (M-IV-10):

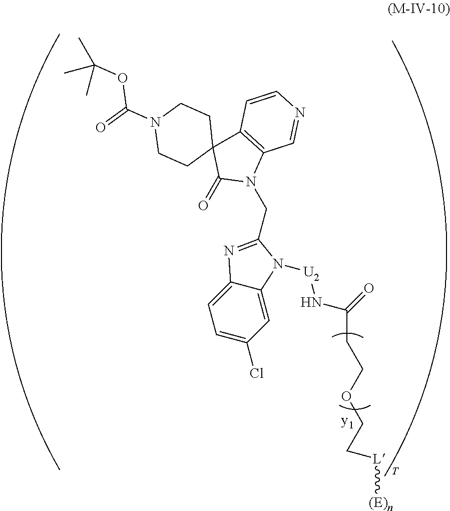

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-IV-11):

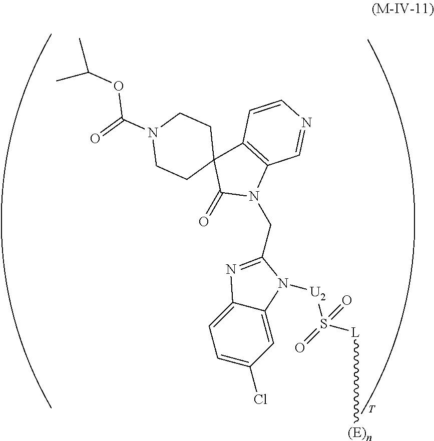

or a pharmaceutically acceptable salt thereof.

In some embodiments, the conjugate is described by formula (M-IV-12):

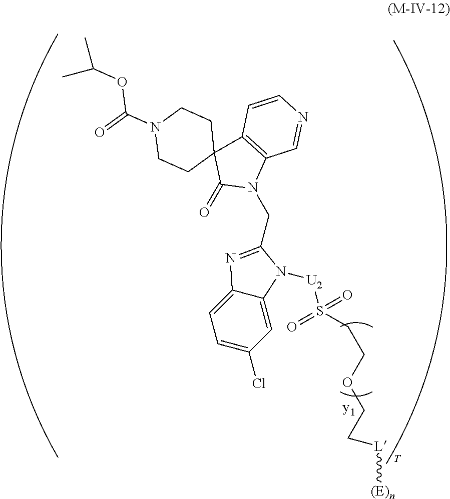

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-IV-13):

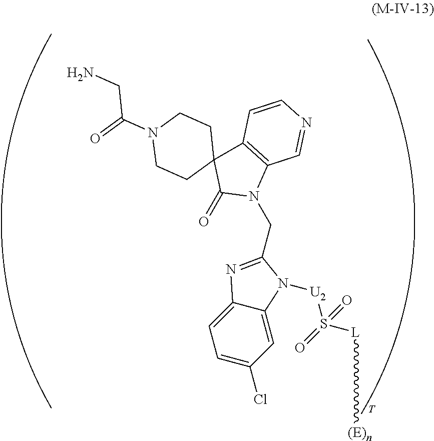

or a pharmaceutically acceptable salt thereof.

In some embodiments, the conjugate is described by formula (M-IV-14):

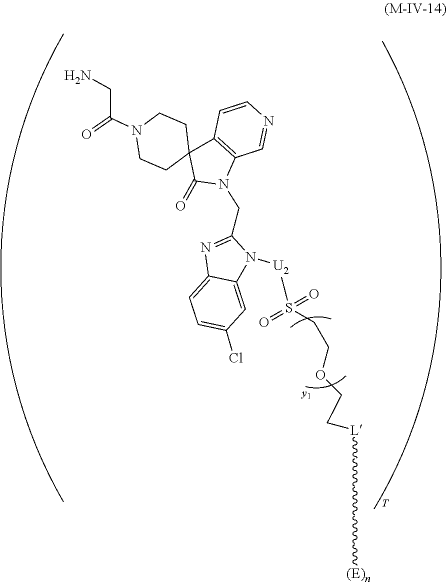

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-IV-15):

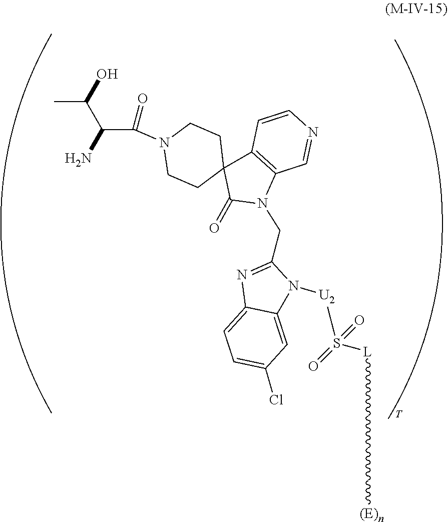

or a pharmaceutically acceptable salt thereof.

In some embodiments, the conjugate is described by formula (M-IV-16):

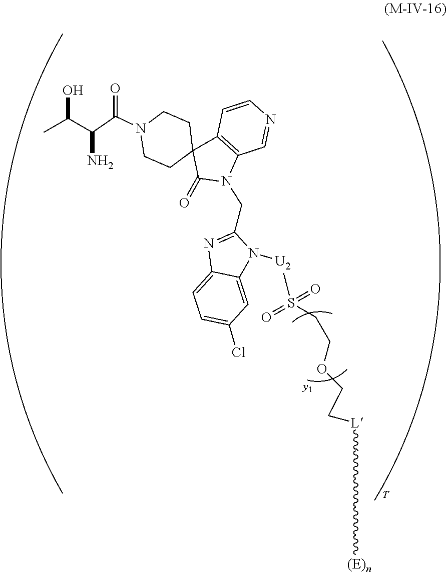

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (M-IV-17):

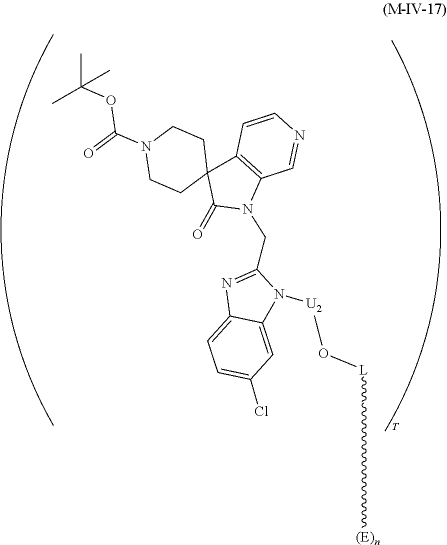

or a pharmaceutically acceptable salt thereof.

In some embodiments, the conjugate is described by formula (M-IV-18):

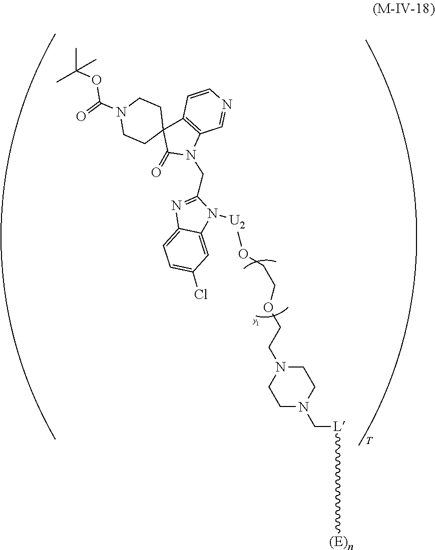

wherein L′ is the remainder of L, and y₁ is an integer from 1-20 (e.g., y₁ is 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments of any of the aspects described herein, L or L′ includes one or more optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₅-C₂₀ heterocycloalkynylene, optionally substituted C₅-C₁₅ arylene, optionally substituted C₃-C₁₅ heteroarylene, O, S, NRⁱ, P, carbonyl, thiocarbonyl, sulfonyl, phosphate, phosphoryl, or imino, wherein Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₃-C₁₅ heteroaryl.

In some embodiments of any of the aspects described herein, the backbone of L or L′ consists of one or more optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene, optionally substituted C₃-C₁₅ heteroarylene, O, S, NRⁱ, P, carbonyl, thiocarbonyl, sulfonyl, phosphate, phosphoryl, or imino, wherein Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₃-C₁₅ heteroaryl.

In some embodiments of any of the aspects described herein, L or L′ is oxo substituted. In some embodiments, the backbone of L or L′ includes no more than 250 atoms. In some embodiments, L or L′ is capable of forming an amide, a carbamate, a sulfonyl, or a urea linkage. In some embodiments, L or L′ is a bond. In some embodiments, L or L′ is an atom. In some embodiments, L′ is a nitrogen atom.

In some embodiments, each L is described by formula (M-L-1):

J¹-(Q¹)_{g}-(T¹)ₕ-(Q²)ᵢ-(T²)ⱼ-(Q³)ₖ-(T³)ₗ-(Q⁴)ₘ-(T⁴)ₙ-(Q⁵)ₒ-J²

wherein: J¹ is a bond attached to A₁; J² is a bond attached to E or a functional group capable of reacting with a functional group conjugated to E (e.g., maleimide and cysteine, amine and activated carboxylic acid, thiol and maleimide, activated sulfonic acid and amine, isocyanate and amine, azide and alkyne, and alkene and tetrazine); each of Q¹, Q², Q³, Q⁴, and Q⁵ is, independently, optionally substituted C₁-C₂₀ alkylene, optionally substituted C₁-C₂₀ heteroalkylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene, or optionally substituted C₃-C₁₅ heteroarylene; each of T¹, T², T³, T⁴ is, independently, O, S, NRⁱ, P, carbonyl, thiocarbonyl, sulfonyl, phosphate, phosphoryl, or imino; Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₃-C₁₅ heteroaryl; and each of g, h, i, j, k, l, m, n, and o is, independently, 0 or 1; or a pharmaceutically acceptable salt thereof.

In some embodiments, optionally substituted includes substitution with a polyethylene glycol (PEG). A PEG has a repeating unit structure (—CH₂CH₂O—)ₙ, wherein n is an integer from 2 to 100. A polyethylene glycol may be selected from any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀).

In some embodiments, J² may have two points of attachment to the Fc domain, Fc-binding peptide, albumin protein, or albumin protein-binding peptide (e.g., two J²).

In some embodiments, L is

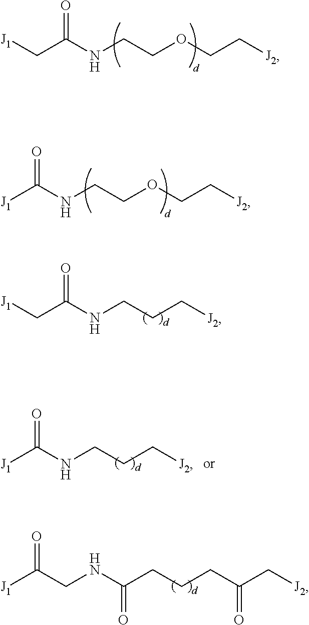

wherein d is an integer from 1 to 20 (e.g., d is 1, 2, 3, 4, 5, 6, 7, 8, 9, 10, 11, 12, 13, 14, 15, 16, 17, 18, 19, or 20).

In some embodiments, L is

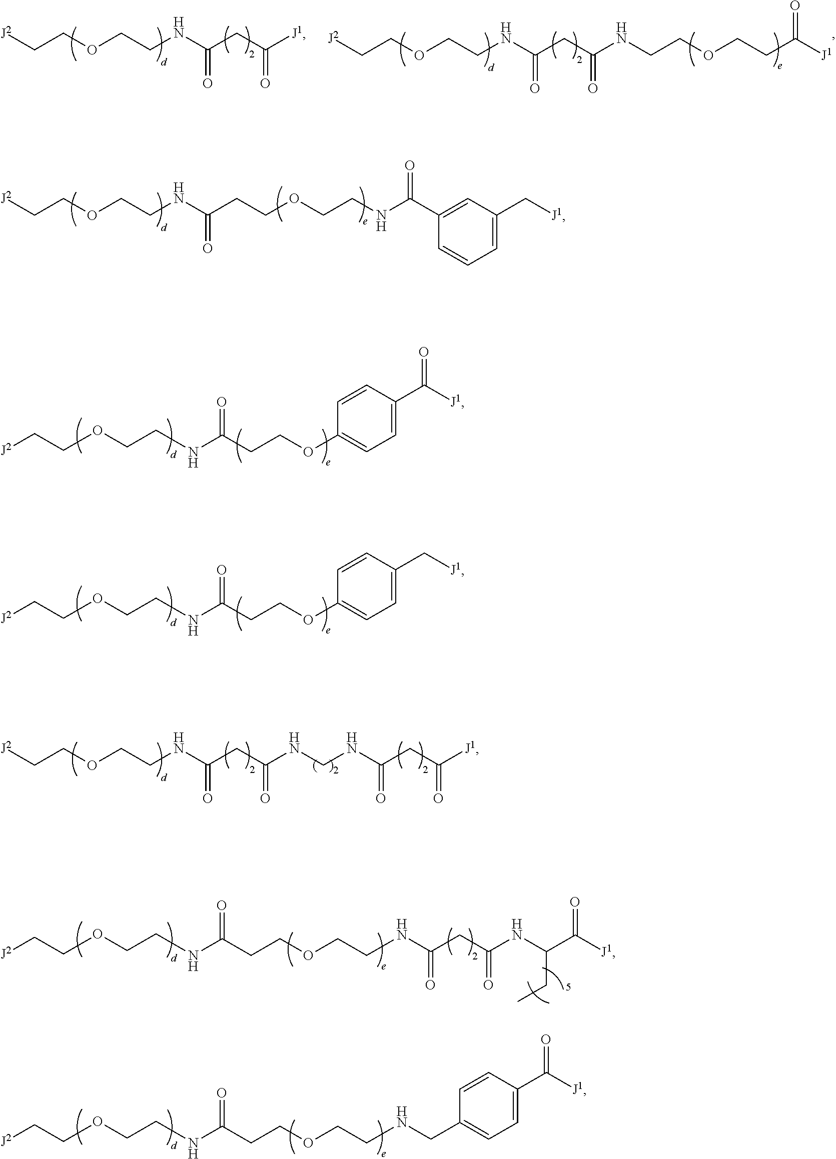

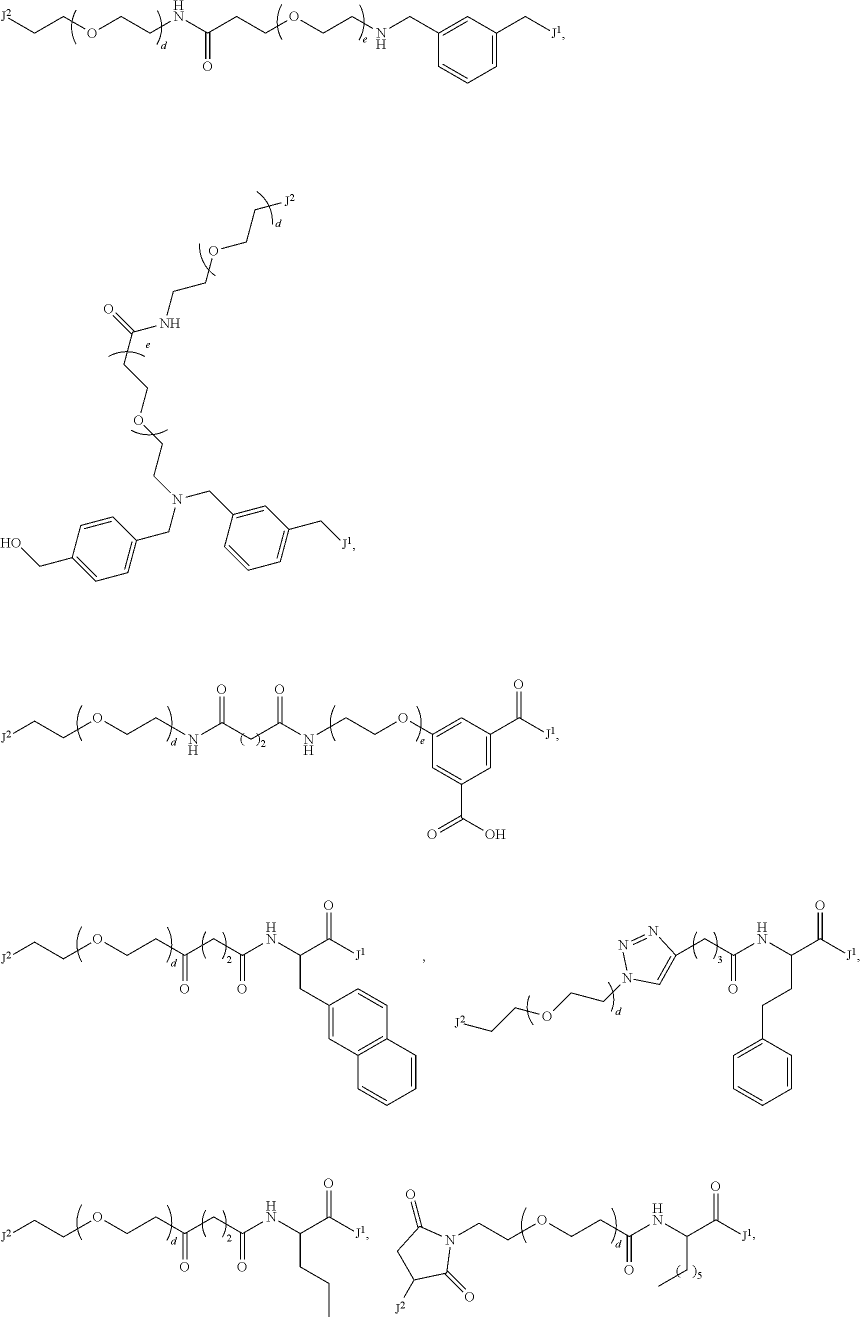

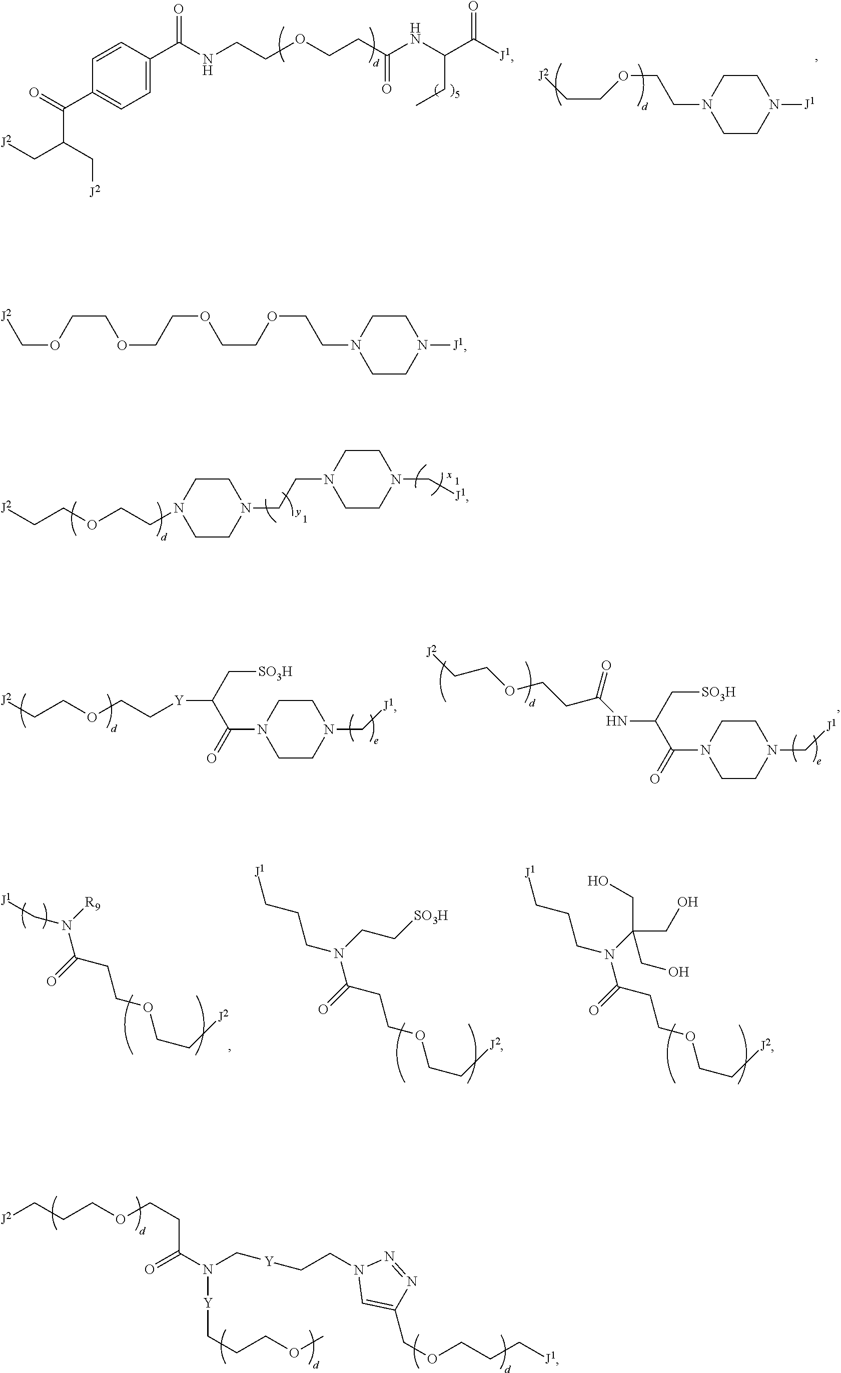

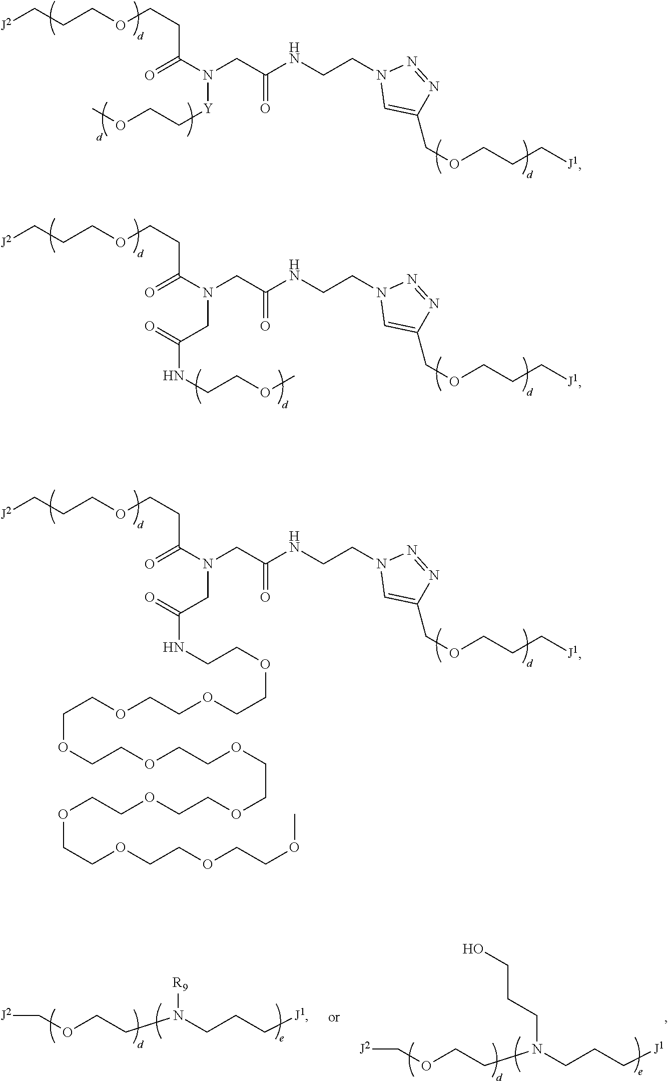

wherein each Y is independently selected from (—O—), (—S—), (—R₈—), (—O(C═O)NR₈—), (—O(C═S)NR₈—), (—O(C═O)O—), (—O(C═O)—), (—NH(C═O)O—), (—NH(C═O)—), (—NH(C═NH)—), (—NH(C═O)NR₈—), (—NH(C═NH)NR₈—), (—NH(C═S)NR₈—), (—NH(C═S)—), (—OCH₂(C═O)NR₈—), (—NH(SO₂)—), (—NH(SO₂)NR₈—), (—OR₉—), (—NR₉—), (—SR₉—), (—R₉NH(C═O)—), (—R₉OR₉C(═O)NH—), (—CH₂NH(C═O)—), (—CH₂OCH₂(C═O)NH—), (—(C═NR₈)NH—), (—NH(SO₂)—), (—(C═O)NH—), (—C(═O)—), (—C(NR₈)—), or (—R₉C(═O)—);

each R₈ is independently selected from H, optionally substituted C₁-C₂₀ alkyl, optionally substituted C₁-C₂₀ alkylene, optionally substituted C₃-C₂₀ cycloalkyl, optionally substituted C₂-C₂₀ heterocycloalkyl, optionally substituted C₅-C₁₅ aryl, and optionally substituted C₂-C₁₅ heteroaryl;

each R₉ is independently selected from optionally substituted C₁-C₂₀ alkylene, optionally substituted C₃-C₂₀ cycloalkyl, optionally substituted C₂-C₂₀ heterocycloalkyl, optionally substituted C₅-C₁₅ aryl, and optionally substituted C₂-C₁₅ heteroaryl; and

each of d, e, y₁, and x₁ is, independently, an integer from 1 to 26 (e.g., d is 1, 2, 3, 4, 5, 6, 7, 8, 9, 10, 11, 12, 13, 14, 15, 16, 17, 18, 19, 20, 21, 22, 23, 24, 25, or 26).

In some embodiments of any of the aspects described herein, L includes a polyethylene glycol (PEG) linker. A PEG linker includes a linker having the repeating unit structure (—CH₂CH₂O—)ₙ, wherein n is an integer from 2 to 100. A polyethylene glycol linker may covalently join a RSV F protein inhibitor and E (e.g., in a conjugate of any one of formulas). A polyethylene glycol linker may covalently join a first RSV F protein inhibitor and a second RSV F protein inhibitor (e.g., in a conjugate of any one of formulas). A polyethylene glycol linker may covalently join a RSV F protein inhibitor dimer and E (e.g., in a conjugate of any one of formulas). A polyethylene glycol linker may selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀). In some embodiments, L^{c} includes a PEG linker, where L^{C} is covalently attached to each of Qⁱ and E.

In some embodiments of any of the aspects described herein, L is covalently attached to the nitrogen atom of a surface exposed lysine of E or L is covalently attached to the sulfur atom of a surface exposed cysteine of E.

In some embodiments of any of the aspects described herein, E is an Fc domain monomer. In some embodiments, n is 2 and each E dimerizes to form an Fc domain.

In some embodiments, n is 2, each E is an Fc domain monomer, each E dimerizes to form an Fc domain, and the conjugate is described by formula (D-I-1):

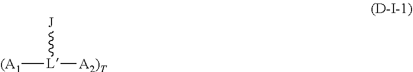

wherein J is an Fc domain; and T is an integer from 1 to 20 (e.g., T is 1, 2, 3, 4, 5, 6, 7, 8, 9, 10, 11, 12, 13, 14, 15, 16, 17, 18, 19, or 20), or a pharmaceutically acceptable salt thereof.

In some embodiments, n is 2, each E is an Fc domain monomer, each E dimerizes to form an Fc domain, and the conjugate is described by formula (M-I-1):

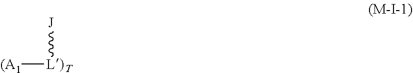

wherein J is an Fc domain; and T is an integer from 1 to 20 (e.g., T is 1, 2, 3, 4, 5, 6, 7, 8, 9, 10, 11, 12, 13, 14, 15, 16, 17, 18, 19, or 20), or a pharmaceutically acceptable salt thereof.

In some embodiments of any of the aspects described herein, E has the sequence of any one of SEQ ID NOs: 1-95.

In some embodiments of any of the aspects described herein, E is an albumin protein, an albumin protein-binding peptide, or an Fc-binding peptide. In some embodiments, where E is an albumin protein, an albumin protein-binding peptide, or an Fc-binding peptide, n is 1.

In some embodiments, n is 1, E is an albumin protein, an albumin protein-binding peptide, or an Fc-binding peptide and the conjugate is described by formula (D-I-2):

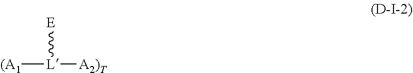

wherein E is an albumin protein, an albumin protein-binding peptide, or Fc-binding peptide; and T is an integer from 1 to 20, or a pharmaceutically acceptable salt thereof.

In some embodiments, n is 1, E is an albumin protein, an albumin protein-binding peptide, or an Fc-binding peptide, and the conjugate is described by formula (M-I-2):

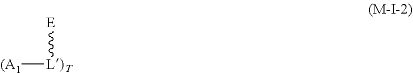

wherein E is an albumin protein, an albumin protein-binding peptide, or an Fc-binding peptide; and T is an integer from 1 to 20, or a pharmaceutically acceptable salt thereof.

In some embodiments of any of the aspects described herein, E is an albumin protein having the sequence of any one of SEQ ID NOs: 96-98.

In some embodiments of any of the aspects described herein, T is 1, 2, 3, 4, or 5.

In another aspect, the invention provides a population of conjugates having the structure of any of the conjugates described herein (e.g., a population of conjugates having the formula of any one of formulas (1), (2), (D-I)-(D-IV), or (M-I)-(M-IV)), wherein the average value of T is 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20.

In another aspect, the invention provides a pharmaceutical composition including any of the conjugates described herein (e.g., a conjugate of any one of formulas (1), (2), (D-I)-(D-IV), or (M-I)-(M-IV)), or a pharmaceutically acceptable salt thereof, and a pharmaceutically acceptable excipient.

In another aspect, the invention provides a method for the treatment of a subject having a viral infection or presumed to have a viral infection, the method including administering to the subject an effective amount of any of the conjugates or compositions described herein (e.g., a conjugate of any one of formulas (1), (2), (D-I)-(D-IV), or (M-I)-(M-IV)).

In another aspect, the invention provides a method for the prophylactic treatment of a viral infection in a subject in need thereof, the method including administering to the subject an effective amount of any of the conjugates or compositions described herein (e.g., a conjugate of any one of formulas (1), (2), (D-I)-(D-IV), or (M-I)-(M-IV)).

In some embodiments, the viral infection is caused by RSV. In some embodiments, the viral infection is RSV A or RSV B.

In some embodiments, the subject is immunocompromised.

In some embodiments, the subject has been diagnosed with humoral immune deficiency, T cell deficiency, neutropenia, asplenia, or complement deficiency.

In some embodiments, the subject is being treated or is about to be treated with an immunosuppressive therapy.

In some embodiments, the subject has been diagnosed with a disease which causes immunosuppression. In some embodiments, the disease is cancer or acquired immunodeficiency syndrome. In some embodiments, the cancer is leukemia, lymphoma, or multiple myeloma.

In some embodiments, the subject has undergone or is about to undergo hematopoietic stem cell transplantation.

In some embodiments, the subject has undergone or is about to undergo an organ transplant.

In some embodiments, the subject is less than 60 months old. In some embodiments, the subject is less than 24 months old. In some embodiments, wherein the subject is a premature infant.

In some embodiments, the conjugate of composition is administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravascularly, mucosally, intrapericardially, intraumbilically, intraocularally, orally, locally, by inhalation, by injection, or by infusion.

In some embodiments, the subject is treated with a second therapeutic agent. In some embodiments, the second therapeutic agent is an antiviral agent. In some embodiments, the second therapeutic agent is a viral vaccine. In some embodiments, the viral vaccine elicits an immune response in the subject against RSV (e.g., RSV A or RSV B).

In some embodiments, an Fc-domain-containing composition may be substituted for an Fc domain and an Fc-domain-monomer-containing composition may be substituted for an Fc domain monomer in any one of formulas (1), (2), (D-I)-(D-IV), or (M-I)-(M-IV) (e.g., any one of formulas (1), (2), (D-I), (D-II), (D-II-1), (D-II-2), (D-II-3), (D-II-4), (D-II-5), (D-II-6), (D-II-7), (D-II-8), (D-II-9), (D-II-10), (D-II-11), (D-II-12), (D-II-13), (D-II-14), (D-II-15), (D-II-16), (D-II-17), (D-III), (D-III-1), (D-III-2), (D-III-3), (D-IV), (D-IV-1), (D-IV-2), (D-IV-3), (D-IV-4), (D-IV-5), (D-IV-6), (D-IV-7), (D-IV-8), (D-IV-9), (D-IV-10), (D-IV-11), (D-IV-12), (D-IV-13), (D-IV-14), (D-IV-15), (D-IV-16), (D-IV-17), (D-IV-18), (M-I), (M-II), (M-II-17), (M-III), (M-III-1), (M-III-2), (M-III-3), (M-IV), (M-IV-1), (M-IV-2), (M-IV-3), (M-IV-4), (M-IV-5), (M-IV-6), (M-IV-7), (M-IV-8), (M-IV-9), (M-IV-10), (M-IV-11), (M-IV-12), (M-IV-13), (M-IV-14), (M-IV-15), (M-IV-16), (M-IV-17), (M-IV-18). In any of the formulas described herein (e.g., any one of formulas (1), (2), (D-I)-(D-IV), or (M-I)-(M-IV)), when n is 1, E is an Fc-domain-monomer-containing composition. In any of the formulas described herein (e.g., any one of formulas (1), (2), (D-I)-(D-IV), or (M-I)-(M-IV)), when n is 2, E is an Fc-domain-containing composition.

In certain embodiments, the Fc-domain-containing composition is an antibody or an antibody fragment. An antibody may include any form of immunoglobulin, heavy chain antibody, light chain antibody, LRR-based antibody, or other protein scaffold with antibody-like properties, as well as any other immunological binding moiety known in the art, including antibody fragments (e.g., a Fab, Fab′, Fab′2, F(ab′)2, Fd, Fv, Feb, scFv, or SMIP). The subunit structures and three-dimensional configurations of different classes of antibodies are known in the art. An antibody fragment may include a binding moiety that includes a portion derived from or having significant homology to an antibody, such as the antigen-determining region of an antibody. Exemplary antibody fragments include Fab, Fab′, Fab′2, F(ab′)2, Fd, Fv, Feb, scFv, and SMIP.

In particular embodiments, the antibody or antibody fragment is a human, mouse, camelid (e.g., llama, alpaca, or camel), goat, sheep, rabbit, chicken, guinea pig, hamster, horse, or rat antibody or antibody fragment. In specific embodiments, the antibody is an IgG, IgA, IgD, IgE, IgM, or intrabody. In certain embodiments, the antibody fragment includes an scFv, sdAb, dAb, Fab, Fab′, Fab′2, F(ab′)2, Fd, Fv, Feb, or SMIP.

In some embodiments, the Fc-domain-containing composition (e.g., an antibody or antibody fragment) confers binding specificity to a one or more targets (e.g., an antigen such as an antigen associated with RSV). RSV targeting antibodies are known in the art, for example, as described in Gilman et al. Sci. Immunol. 1(6), (2006), which is incorporated herein by reference in its entirety.

In some embodiments, the one or more targets (e.g., an antigen) bound by the Fc-domain-containing composition (e.g., an antibody or antibody fragment) is a viral (e.g., RSV) protein such as RSV F protein. In some embodiments, the antibody or antibody fragment recognizes a viral surface antigen.

In some embodiments of any of the aspects described herein, E (e.g., each E) includes the amino acid sequence of SEQ ID NO: 1. In some embodiments, E includes an amino acid sequence that is at least 70%, 75%, 80%, 85%, 90%, 95%, 96%, 97%, 98%, 99%, or 100% identical to the amino acid sequence of SEQ ID NO: 1.

In some embodiments of any of the aspects described herein, E (e.g., each E) includes the amino acid sequence of SEQ ID NO: 2. In some embodiments, E includes an amino acid sequence that is at least 70%, 75%, 80%, 85%, 90%, 95%, 96%, 97%, 98%, 99%, or 100% identical to the amino acid sequence of SEQ ID NO: 2.

In some embodiments of any of the aspects described herein, E (e.g., each E) includes the amino acid sequence of SEQ ID NO: 3. In some embodiments, E includes an amino acid sequence that is at least 70%, 75%, 80%, 85%, 90%, 95%, 96%, 97%, 98%, 99%, or 100% identical to the amino acid sequence of SEQ ID NO: 3.

In some embodiments of any of the aspects described herein, E (e.g., each E) includes the amino acid sequence of SEQ ID NO: 4. In some embodiments, E includes an amino acid sequence that is at least 70%, 75%, 80%, 85%, 90%, 95%, 96%, 97%, 98%, 99%, or 100% identical to the amino acid sequence of SEQ ID NO: 4.

In some embodiments of any of the aspects described herein, E (e.g., each E) includes the amino acid sequence of SEQ ID NO: 5. In some embodiments, E includes an amino acid sequence that is at least 70%, 75%, 80%, 85%, 90%, 95%, 96%, 97%, 98%, 99%, or 100% identical to the amino acid sequence of SEQ ID NO: 5.

In some embodiments of any of the aspects described herein, E (e.g., each E) includes the amino acid sequence of SEQ ID NO: 6. In some embodiments, E includes an amino acid sequence that is at least 70%, 75%, 80%, 85%, 90%, 95%, 96%, 97%, 98%, 99%, or 100% identical to the amino acid sequence of SEQ ID NO: 6.

In some embodiments of any of the aspects described herein, E (e.g., each E) includes the amino acid sequence of SEQ ID NO: 7. In some embodiments, E includes an amino acid sequence that is at least 70%, 75%, 80%, 85%, 90%, 95%, 96%, 97%, 98%, 99%, or 100% identical to the amino acid sequence of SEQ ID NO: 7.

In some embodiments of any of the aspects described herein, E (e.g., each E) includes the amino acid sequence of SEQ ID NO: 8. In some embodiments, E includes an amino acid sequence that is at least 70%, 75%, 80%, 85%, 90%, 95%, 96%, 97%, 98%, 99%, or 100% identical to the amino acid sequence of SEQ ID NO: 8.

In some embodiments of any of the aspects described herein, E (e.g., each E) includes the amino acid sequence of SEQ ID NO: 9. In some embodiments, E includes an amino acid sequence that is at least 70%, 75%, 80%, 85%, 90%, 95%, 96%, 97%, 98%, 99%, or 100% identical to the amino acid sequence of SEQ ID NO: 9.

In some embodiments of any of the aspects described herein, E (e.g., each E) includes the amino acid sequence of SEQ ID NO: 10. In some embodiments, E includes an amino acid sequence that is at least 70%, 75%, 80%, 85%, 90%, 95%, 96%, 97%, 98%, 99%, or 100% identical to the amino acid sequence of SEQ ID NO: 10.

In some embodiments of any of the aspects described herein, E (e.g., each E) includes the amino acid sequence of SEQ ID NO: 11. In some embodiments, E includes an amino acid sequence that is at least 70%, 75%, 80%, 85%, 90%, 95%, 96%, 97%, 98%, 99%, or 100% identical to the amino acid sequence of SEQ ID NO: 11.

In some embodiments of any of the aspects described herein, E (e.g., each E) includes the amino acid sequence of SEQ ID NO: 12. In some embodiments, E includes an amino acid sequence that is at least 70%, 75%, 80%, 85%, 90%, 95%, 96%, 97%, 98%, 99%, or 100% identical to the amino acid sequence of SEQ ID NO: 12.

In some embodiments of any of the aspects described herein, E (e.g., each E) includes the amino acid sequence of SEQ ID NO: 13. In some embodiments, E includes an amino acid sequence that is at least 70%, 75%, 80%, 85%, 90%, 95%, 96%, 97%, 98%, 99%, or 100% identical to the amino acid sequence of SEQ ID NO: 13.

In some embodiments of any of the aspects described herein, E (e.g., each E) includes the amino acid sequence of SEQ ID NO: 14. In some embodiments, E includes an amino acid sequence that is at least 70%, 75%, 80%, 85%, 90%, 95%, 96%, 97%, 98%, 99%, or 100% identical to the amino acid sequence of SEQ ID NO: 14.

In some embodiments of any of the aspects described herein, E (e.g., each E) includes the amino acid sequence of SEQ ID NO: 15. In some embodiments, E includes an amino acid sequence that is at least 70%, 75%, 80%, 85%, 90%, 95%, 96%, 97%, 98%, 99%, or 100% identical to the amino acid sequence of SEQ ID NO: 15.

In some embodiments of any of the aspects described herein, E (e.g., each E) includes the amino acid sequence of SEQ ID NO: 16. In some embodiments, E includes an amino acid sequence that is at least 70%, 75%, 80%, 85%, 90%, 95%, 96%, 97%, 98%, 99%, or 100% identical to the amino acid sequence of SEQ ID NO: 16.

In some embodiments of any of the aspects described herein, E (e.g., each E) includes the amino acid sequence of SEQ ID NO: 17. In some embodiments, E includes an amino acid sequence that is at least 70%, 75%, 80%, 85%, 90%, 95%, 96%, 97%, 98%, 99%, or 100% identical to the amino acid sequence of SEQ ID NO: 17.

In some embodiments of any of the aspects described herein, E (e.g., each E) includes the amino acid sequence of SEQ ID NO: 18. In some embodiments, E includes an amino acid sequence that is at least 70%, 75%, 80%, 85%, 90%, 95%, 96%, 97%, 98%, 99%, or 100% identical to the amino acid sequence of SEQ ID NO: 18.

In some embodiments of any of the aspects described herein, E (e.g., each E) includes the amino acid sequence of SEQ ID NO: 19. In some embodiments, E includes an amino acid sequence that is at least 70%, 75%, 80%, 85%, 90%, 95%, 96%, 97%, 98%, 99%, or 100% identical to the amino acid sequence of SEQ ID NO: 19.

In some embodiments of any of the aspects described herein, E (e.g., each E) includes the amino acid sequence of SEQ ID NO: 20. In some embodiments, E includes an amino acid sequence that is at least 70%, 75%, 80%, 85%, 90%, 95%, 96%, 97%, 98%, 99%, or 100% identical to the amino acid sequence of SEQ ID NO: 20.

In some embodiments of any of the aspects described herein, E (e.g., each E) includes the amino acid sequence of SEQ ID NO: 21. In some embodiments, E includes an amino acid sequence that is at least 70%, 75%, 80%, 85%, 90%, 95%, 96%, 97%, 98%, 99%, or 100% identical to the amino acid sequence of SEQ ID NO: 21.

In some embodiments of any of the aspects described herein, E (e.g., each E) includes the amino acid sequence of SEQ ID NO: 22. In some embodiments, E includes an amino acid sequence that is at least 70%, 75%, 80%, 85%, 90%, 95%, 96%, 97%, 98%, 99%, or 100% identical to the amino acid sequence of SEQ ID NO: 22.

In some embodiments of any of the aspects described herein, E (e.g., each E) includes the amino acid sequence of SEQ ID NO: 23. In some embodiments, E includes an amino acid sequence that is at least 70%, 75%, 80%, 85%, 90%, 95%, 96%, 97%, 98%, 99%, or 100% identical to the amino acid sequence of SEQ ID NO: 23.

In some embodiments of any of the aspects described herein, E (e.g., each E) includes the amino acid sequence of SEQ ID NO: 24. In some embodiments, E includes an amino acid sequence that is at least 70%, 75%, 80%, 85%, 90%, 95%, 96%, 97%, 98%, 99%, or 100% identical to the amino acid sequence of SEQ ID NO: 24.

In some embodiments of any of the aspects described herein, E (e.g., each E) includes the amino acid sequence of SEQ ID NO: 25. In some embodiments, E includes an amino acid sequence that is at least 70%, 75%, 80%, 85%, 90%, 95%, 96%, 97%, 98%, 99%, or 100% identical to the amino acid sequence of SEQ ID NO: 25.

In some embodiments of any of the aspects described herein, E (e.g., each E) includes the amino acid sequence of SEQ ID NO: 26. In some embodiments, E includes an amino acid sequence that is at least 70%, 75%, 80%, 85%, 90%, 95%, 96%, 97%, 98%, 99%, or 100% identical to the amino acid sequence of SEQ ID NO: 26.

In some embodiments of any of the aspects described herein, E (e.g., each E) includes the amino acid sequence of SEQ ID NO: 27. In some embodiments, E includes an amino acid sequence that is at least 70%, 75%, 80%, 85%, 90%, 95%, 96%, 97%, 98%, 99%, or 100% identical to the amino acid sequence of SEQ ID NO: 27.

In some embodiments of any of the aspects described herein, E (e.g., each E) includes the amino acid sequence of SEQ ID NO: 28. In some embodiments, E includes an amino acid sequence that is at least 70%, 75%, 80%, 85%, 90%, 95%, 96%, 97%, 98%, 99%, or 100% identical to the amino acid sequence of SEQ ID NO: 28.

In some embodiments of any of the aspects described herein, E (e.g., each E) includes the amino acid sequence of SEQ ID NO: 29. In some embodiments, E includes an amino acid sequence that is at least 70%, 75%, 80%, 85%, 90%, 95%, 96%, 97%, 98%, 99%, or 100% identical to the amino acid sequence of SEQ ID NO: 29.

In some embodiments of any of the aspects described herein, E (e.g., each E) includes the amino acid sequence of SEQ ID NO: 30. In some embodiments, E includes an amino acid sequence that is at least 70%, 75%, 80%, 85%, 90%, 95%, 96%, 97%, 98%, 99%, or 100% identical to the amino acid sequence of SEQ ID NO: 30.

In some embodiments of any of the aspects described herein, E (e.g., each E) includes the amino acid sequence of SEQ ID NO: 31. In some embodiments, E includes an amino acid sequence that is at least 70%, 75%, 80%, 85%, 90%, 95%, 96%, 97%, 98%, 99%, or 100% identical to the amino acid sequence of SEQ ID NO: 31.

In some embodiments of any of the aspects described herein, E (e.g., each E) includes the amino acid sequence of SEQ ID NO: 32. In some embodiments, E includes an amino acid sequence that is at least 70%, 75%, 80%, 85%, 90%, 95%, 96%, 97%, 98%, 99%, or 100% identical to the amino acid sequence of SEQ ID NO: 32.

In some embodiments of any of the aspects described herein, E (e.g., each E) includes the amino acid sequence of SEQ ID NO: 33. In some embodiments, E includes an amino acid sequence that is at least 70%, 75%, 80%, 85%, 90%, 95%, 96%, 97%, 98%, 99%, or 100% identical to the amino acid sequence of SEQ ID NO: 33.

In some embodiments of any of the aspects described herein, E (e.g., each E) includes the amino acid sequence of SEQ ID NO: 34. In some embodiments, E includes an amino acid sequence that is at least 70%, 75%, 80%, 85%, 90%, 95%, 96%, 97%, 98%, 99%, or 100% identical to the amino acid sequence of SEQ ID NO: 34.

In some embodiments of any of the aspects described herein, E (e.g., each E) includes the amino acid sequence of SEQ ID NO: 35. In some embodiments, E includes an amino acid sequence that is at least 70%, 75%, 80%, 85%, 90%, 95%, 96%, 97%, 98%, 99%, or 100% identical to the amino acid sequence of SEQ ID NO: 35.

In some embodiments of any of the aspects described herein, E (e.g., each E) includes the amino acid sequence of SEQ ID NO: 36. In some embodiments, E includes an amino acid sequence that is at least 70%, 75%, 80%, 85%, 90%, 95%, 96%, 97%, 98%, 99%, or 100% identical to the amino acid sequence of SEQ ID NO: 36.

In some embodiments of any of the aspects described herein, E (e.g., each E) includes the amino acid sequence of SEQ ID NO: 37. In some embodiments, E includes an amino acid sequence that is at least 70%, 75%, 80%, 85%, 90%, 95%, 96%, 97%, 98%, 99%, or 100% identical to the amino acid sequence of SEQ ID NO: 37.

In some embodiments of any of the aspects described herein, E (e.g., each E) includes the amino acid sequence of SEQ ID NO: 38. In some embodiments, E includes an amino acid sequence that is at least 70%, 75%, 80%, 85%, 90%, 95%, 96%, 97%, 98%, 99%, or 100% identical to the amino acid sequence of SEQ ID NO: 38.

In some embodiments of any of the aspects described herein, E (e.g., each E) includes the amino acid sequence of SEQ ID NO: 39. In some embodiments, E includes an amino acid sequence that is at least 70%, 75%, 80%, 85%, 90%, 95%, 96%, 97%, 98%, 99%, or 100% identical to the amino acid sequence of SEQ ID NO: 39.

In some embodiments of any of the aspects described herein, E (e.g., each E) includes the amino acid sequence of SEQ ID NO: 40. In some embodiments, E includes an amino acid sequence that is at least 70%, 75%, 80%, 85%, 90%, 95%, 96%, 97%, 98%, 99%, or 100% identical to the amino acid sequence of SEQ ID NO: 40.

In some embodiments of any of the aspects described herein, E (e.g., each E) includes the amino acid sequence of SEQ ID NO: 41. In some embodiments, E includes an amino acid sequence that is at least 70%, 75%, 80%, 85%, 90%, 95%, 96%, 97%, 98%, 99%, or 100% identical to the amino acid sequence of SEQ ID NO: 41.

In some embodiments of any of the aspects described herein, E (e.g., each E) includes the amino acid sequence of SEQ ID NO: 42. In some embodiments, E includes an amino acid sequence that is at least 70%, 75%, 80%, 85%, 90%, 95%, 96%, 97%, 98%, 99%, or 100% identical to the amino acid sequence of SEQ ID NO: 42.

In some embodiments of any of the aspects described herein, E (e.g., each E) includes the amino acid sequence of SEQ ID NO: 43. In some embodiments, E includes an amino acid sequence that is at least 70%, 75%, 80%, 85%, 90%, 95%, 96%, 97%, 98%, 99%, or 100% identical to the amino acid sequence of SEQ ID NO: 43.

In some embodiments of any of the aspects described herein, E (e.g., each E) includes the amino acid sequence of SEQ ID NO: 44. In some embodiments, E includes an amino acid sequence that is at least 70%, 75%, 80%, 85%, 90%, 95%, 96%, 97%, 98%, 99%, or 100% identical to the amino acid sequence of SEQ ID NO: 45.

In some embodiments of any of the aspects described herein, E (e.g., each E) includes the amino acid sequence of SEQ ID NO: 46. In some embodiments, E includes an amino acid sequence that is at least 70%, 75%, 80%, 85%, 90%, 95%, 96%, 97%, 98%, 99%, or 100% identical to the amino acid sequence of SEQ ID NO: 46.

In some embodiments of any of the aspects described herein, E (e.g., each E) includes the amino acid sequence of SEQ ID NO: 47. In some embodiments, E includes an amino acid sequence that is at least 70%, 75%, 80%, 85%, 90%, 95%, 96%, 97%, 98%, 99%, or 100% identical to the amino acid sequence of SEQ ID NO: 47.

In some embodiments of any of the aspects described herein, E (e.g., each E) includes the amino acid sequence of SEQ ID NO: 48. In some embodiments, E includes an amino acid sequence that is at least 70%, 75%, 80%, 85%, 90%, 95%, 96%, 97%, 98%, 99%, or 100% identical to the amino acid sequence of SEQ ID NO: 48.

In some embodiments of any of the aspects described herein, E (e.g., each E) includes the amino acid sequence of SEQ ID NO: 49. In some embodiments, E includes an amino acid sequence that is at least 70%, 75%, 80%, 85%, 90%, 95%, 96%, 97%, 98%, 99%, or 100% identical to the amino acid sequence of SEQ ID NO: 49.

In some embodiments of any of the aspects described herein, E (e.g., each E) includes the amino acid sequence of SEQ ID NO: 50. In some embodiments, E includes an amino acid sequence that is at least 70%, 75%, 80%, 85%, 90%, 95%, 96%, 97%, 98%, 99%, or 100% identical to the amino acid sequence of SEQ ID NO: 50.

In some embodiments of any of the aspects described herein, E (e.g., each E) includes the amino acid sequence of SEQ ID NO: 51. In some embodiments, E includes an amino acid sequence that is at least 70%, 75%, 80%, 85%, 90%, 95%, 96%, 97%, 98%, 99%, or 100% identical to the amino acid sequence of SEQ ID NO: 51.

In some embodiments of any of the aspects described herein, E (e.g., each E) includes the amino acid sequence of SEQ ID NO: 52. In some embodiments, E includes an amino acid sequence that is at least 70%, 75%, 80%, 85%, 90%, 95%, 96%, 97%, 98%, 99%, or 100% identical to the amino acid sequence of SEQ ID NO: 52.

In some embodiments of any of the aspects described herein, E (e.g., each E) includes the amino acid sequence of SEQ ID NO: 53. In some embodiments, E includes an amino acid sequence that is at least 70%, 75%, 80%, 85%, 90%, 95%, 96%, 97%, 98%, 99%, or 100% identical to the amino acid sequence of SEQ ID NO: 53.

In some embodiments of any of the aspects described herein, E (e.g., each E) includes the amino acid sequence of SEQ ID NO: 54. In some embodiments, E includes an amino acid sequence that is at least 70%, 75%, 80%, 85%, 90%, 95%, 96%, 97%, 98%, 99%, or 100% identical to the amino acid sequence of SEQ ID NO: 54.

In some embodiments of any of the aspects described herein, E (e.g., each E) includes the amino acid sequence of SEQ ID NO: 55. In some embodiments, E includes an amino acid sequence that is at least 70%, 75%, 80%, 85%, 90%, 95%, 96%, 97%, 98%, 99%, or 100% identical to the amino acid sequence of SEQ ID NO: 55.

In some embodiments of any of the aspects described herein, E (e.g., each E) includes the amino acid sequence of SEQ ID NO: 56. In some embodiments, E includes an amino acid sequence that is at least 70%, 75%, 80%, 85%, 90%, 95%, 96%, 97%, 98%, 99%, or 100% identical to the amino acid sequence of SEQ ID NO: 56.

In some embodiments of any of the aspects described herein, E (e.g., each E) includes the amino acid sequence of SEQ ID NO: 57. In some embodiments, E includes an amino acid sequence that is at least 70%, 75%, 80%, 85%, 90%, 95%, 96%, 97%, 98%, 99%, or 100% identical to the amino acid sequence of SEQ ID NO: 57.

In some embodiments of any of the aspects described herein, E (e.g., each E) includes the amino acid sequence of SEQ ID NO: 58. In some embodiments, E includes an amino acid sequence that is at least 70%, 75%, 80%, 85%, 90%, 95%, 96%, 97%, 98%, 99%, or 100% identical to the amino acid sequence of SEQ ID NO: 58.

In some embodiments of any of the aspects described herein, E (e.g., each E) includes the amino acid sequence of SEQ ID NO: 59. In some embodiments, E includes an amino acid sequence that is at least 70%, 75%, 80%, 85%, 90%, 95%, 96%, 97%, 98%, 99%, or 100% identical to the amino acid sequence of SEQ ID NO: 59.

In some embodiments of any of the aspects described herein, E (e.g., each E) includes the amino acid sequence of SEQ ID NO: 60. In some embodiments, E includes an amino acid sequence that is at least 70%, 75%, 80%, 85%, 90%, 95%, 96%, 97%, 98%, 99%, or 100% identical to the amino acid sequence of SEQ ID NO: 60.

In some embodiments of any of the aspects described herein, E (e.g., each E) includes the amino acid sequence of SEQ ID NO: 61. In some embodiments, E includes an amino acid sequence that is at least 70%, 75%, 80%, 85%, 90%, 95%, 96%, 97%, 98%, 99%, or 100% identical to the amino acid sequence of SEQ ID NO: 61.

In some embodiments of any of the aspects described herein, E (e.g., each E) includes the amino acid sequence of SEQ ID NO: 62. In some embodiments, E includes an amino acid sequence that is at least 70%, 75%, 80%, 85%, 90%, 95%, 96%, 97%, 98%, 99%, or 100% identical to the amino acid sequence of SEQ ID NO: 62.

In some embodiments of any of the aspects described herein, E (e.g., each E) includes the amino acid sequence of SEQ ID NO: 63. In some embodiments, E includes an amino acid sequence that is at least 70%, 75%, 80%, 85%, 90%, 95%, 96%, 97%, 98%, 99%, or 100% identical to the amino acid sequence of SEQ ID NO: 63.

In some embodiments of any of the aspects described herein, E (e.g., each E) includes the amino acid sequence of SEQ ID NO: 64. In some embodiments, E includes an amino acid sequence that is at least 70%, 75%, 80%, 85%, 90%, 95%, 96%, 97%, 98%, 99%, or 100% identical to the amino acid sequence of SEQ ID NO: 64.

In some embodiments of any of the aspects described herein, E (e.g., each E) includes the amino acid sequence of SEQ ID NO: 65. In some embodiments, E includes an amino acid sequence that is at least 70%, 75%, 80%, 85%, 90%, 95%, 96%, 97%, 98%, 99%, or 100% identical to the amino acid sequence of SEQ ID NO: 65.

In some embodiments of any of the aspects described herein, E (e.g., each E) includes the amino acid sequence of SEQ ID NO: 66. In some embodiments, E includes an amino acid sequence that is at least 70%, 75%, 80%, 85%, 90%, 95%, 96%, 97%, 98%, 99%, or 100% identical to the amino acid sequence of SEQ ID NO: 66.

In some embodiments of any of the aspects described herein, E (e.g., each E) includes the amino acid sequence of SEQ ID NO: 67. In some embodiments, E includes an amino acid sequence that is at least 70%, 75%, 80%, 85%, 90%, 95%, 96%, 97%, 98%, 99%, or 100% identical to the amino acid sequence of SEQ ID NO: 67.

In some embodiments of any of the aspects described herein, E (e.g., each E) includes the amino acid sequence of SEQ ID NO: 68. In some embodiments, E includes an amino acid sequence that is at least 70%, 75%, 80%, 85%, 90%, 95%, 96%, 97%, 98%, 99%, or 100% identical to the amino acid sequence of SEQ ID NO: 68.

In some embodiments of any of the aspects described herein, E (e.g., each E) includes the amino acid sequence of SEQ ID NO: 69. In some embodiments, E includes an amino acid sequence that is at least 70%, 75%, 80%, 85%, 90%, 95%, 96%, 97%, 98%, 99%, or 100% identical to the amino acid sequence of SEQ ID NO: 69.

In some embodiments of any of the aspects described herein, E (e.g., each E) includes the amino acid sequence of SEQ ID NO: 70. In some embodiments, E includes an amino acid sequence that is at least 70%, 75%, 80%, 85%, 90%, 95%, 96%, 97%, 98%, 99%, or 100% identical to the amino acid sequence of SEQ ID NO: 70.

In some embodiments of any of the aspects described herein, E (e.g., each E) includes the amino acid sequence of SEQ ID NO: 71. In some embodiments, E includes an amino acid sequence that is at least 70%, 75%, 80%, 85%, 90%, 95%, 96%, 97%, 98%, 99%, or 100% identical to the amino acid sequence of SEQ ID NO: 71.

In some embodiments of any of the aspects described herein, E (e.g., each E) includes the amino acid sequence of SEQ ID NO: 72. In some embodiments, E includes an amino acid sequence that is at least 70%, 75%, 80%, 85%, 90%, 95%, 96%, 97%, 98%, 99%, or 100% identical to the amino acid sequence of SEQ ID NO: 72.

In some embodiments of any of the aspects described herein, E (e.g., each E) includes the amino acid sequence of SEQ ID NO: 73. In some embodiments, E includes an amino acid sequence that is at least 70%, 75%, 80%, 85%, 90%, 95%, 96%, 97%, 98%, 99%, or 100% identical to the amino acid sequence of SEQ ID NO: 73.

In some embodiments of any of the aspects described herein, E (e.g., each E) includes the amino acid sequence of SEQ ID NO: 74. In some embodiments, E includes an amino acid sequence that is at least 70%, 75%, 80%, 85%, 90%, 95%, 96%, 97%, 98%, 99%, or 100% identical to the amino acid sequence of SEQ ID NO: 74.

In some embodiments of any of the aspects described herein, E (e.g., each E) includes the amino acid sequence of SEQ ID NO: 75. In some embodiments, E includes an amino acid sequence that is at least 70%, 75%, 80%, 85%, 90%, 95%, 96%, 97%, 98%, 99%, or 100% identical to the amino acid sequence of SEQ ID NO: 75.

In some embodiments of any of the aspects described herein, E (e.g., each E) includes the amino acid sequence of SEQ ID NO: 76. In some embodiments, E includes an amino acid sequence that is at least 70%, 75%, 80%, 85%, 90%, 95%, 96%, 97%, 98%, 99%, or 100% identical to the amino acid sequence of SEQ ID NO: 76.

In some embodiments of any of the aspects described herein, E (e.g., each E) includes the amino acid sequence of SEQ ID NO: 77. In some embodiments, E includes an amino acid sequence that is at least 70%, 75%, 80%, 85%, 90%, 95%, 96%, 97%, 98%, 99%, or 100% identical to the amino acid sequence of SEQ ID NO: 77.

In some embodiments of any of the aspects described herein, E (e.g., each E) includes the amino acid sequence of SEQ ID NO: 78. In some embodiments, E includes an amino acid sequence that is at least 70%, 75%, 80%, 85%, 90%, 95%, 96%, 97%, 98%, 99%, or 100% identical to the amino acid sequence of SEQ ID NO: 78.

In some embodiments of any of the aspects described herein, E (e.g., each E) includes the amino acid sequence of SEQ ID NO: 79. In some embodiments, E includes an amino acid sequence that is at least 70%, 75%, 80%, 85%, 90%, 95%, 96%, 97%, 98%, 99%, or 100% identical to the amino acid sequence of SEQ ID NO: 79.

In some embodiments of any of the aspects described herein, E (e.g., each E) includes the amino acid sequence of SEQ ID NO: 80. In some embodiments, E includes an amino acid sequence that is at least 70%, 75%, 80%, 85%, 90%, 95%, 96%, 97%, 98%, 99%, or 100% identical to the amino acid sequence of SEQ ID NO: 80.

In some embodiments of any of the aspects described herein, E (e.g., each E) includes the amino acid sequence of SEQ ID NO: 81. In some embodiments, E includes an amino acid sequence that is at least 70%, 75%, 80%, 85%, 90%, 95%, 96%, 97%, 98%, 99%, or 100% identical to the amino acid sequence of SEQ ID NO: 81.

In some embodiments of any of the aspects described herein, E (e.g., each E) includes the amino acid sequence of SEQ ID NO: 82. In some embodiments, E includes an amino acid sequence that is at least 70%, 75%, 80%, 85%, 90%, 95%, 96%, 97%, 98%, 99%, or 100% identical to the amino acid sequence of SEQ ID NO: 82.

In some embodiments of any of the aspects described herein, E (e.g., each E) includes the amino acid sequence of SEQ ID NO: 83. In some embodiments, E includes an amino acid sequence that is at least 70%, 75%, 80%, 85%, 90%, 95%, 96%, 97%, 98%, 99%, or 100% identical to the amino acid sequence of SEQ ID NO: 83.

In some embodiments of any of the aspects described herein, E (e.g., each E) includes the amino acid sequence of SEQ ID NO: 84. In some embodiments, E includes an amino acid sequence that is at least 70%, 75%, 80%, 85%, 90%, 95%, 96%, 97%, 98%, 99%, or 100% identical to the amino acid sequence of SEQ ID NO: 84.

In some embodiments of any of the aspects described herein, E (e.g., each E) includes the amino acid sequence of SEQ ID NO: 85. In some embodiments, E includes an amino acid sequence that is at least 70%, 75%, 80%, 85%, 90%, 95%, 96%, 97%, 98%, 99%, or 100% identical to the amino acid sequence of SEQ ID NO: 85.

In some embodiments of any of the aspects described herein, E (e.g., each E) includes the amino acid sequence of SEQ ID NO: 86. In some embodiments, E includes an amino acid sequence that is at least 70%, 75%, 80%, 85%, 90%, 95%, 96%, 97%, 98%, 99%, or 100% identical to the amino acid sequence of SEQ ID NO: 86.

In some embodiments of any of the aspects described herein, E (e.g., each E) includes the amino acid sequence of SEQ ID NO: 87. In some embodiments, E includes an amino acid sequence that is at least 70%, 75%, 80%, 85%, 90%, 95%, 96%, 97%, 98%, 99%, or 100% identical to the amino acid sequence of SEQ ID NO: 87.

In some embodiments of any of the aspects described herein, E (e.g., each E) includes the amino acid sequence of SEQ ID NO: 88. In some embodiments, E includes an amino acid sequence that is at least 70%, 75%, 80%, 85%, 90%, 95%, 96%, 97%, 98%, 99%, or 100% identical to the amino acid sequence of SEQ ID NO: 88.

In some embodiments of any of the aspects described herein, E (e.g., each E) includes the amino acid sequence of SEQ ID NO: 89. In some embodiments, E includes an amino acid sequence that is at least 70%, 75%, 80%, 85%, 90%, 95%, 96%, 97%, 98%, 99%, or 100% identical to the amino acid sequence of SEQ ID NO: 89.

In some embodiments of any of the aspects described herein, E (e.g., each E) includes the amino acid sequence of SEQ ID NO: 90. In some embodiments, E includes an amino acid sequence that is at least 70%, 75%, 80%, 85%, 90%, 95%, 96%, 97%, 98%, 99%, or 100% identical to the amino acid sequence of SEQ ID NO: 90.

In some embodiments of any of the aspects described herein, E (e.g., each E) includes the amino acid sequence of SEQ ID NO: 91. In some embodiments, E includes an amino acid sequence that is at least 70%, 75%, 80%, 85%, 90%, 95%, 96%, 97%, 98%, 99%, or 100% identical to the amino acid sequence of SEQ ID NO: 91.

In some embodiments of any of the aspects described herein, E (e.g., each E) includes the amino acid sequence of SEQ ID NO: 92. In some embodiments, E includes an amino acid sequence that is at least 70%, 75%, 80%, 85%, 90%, 95%, 96%, 97%, 98%, 99%, or 100% identical to the amino acid sequence of SEQ ID NO: 92.

In some embodiments of any of the aspects described herein, E (e.g., each E) includes the amino acid sequence of SEQ ID NO: 93. In some embodiments, E includes an amino acid sequence that is at least 70%, 75%, 80%, 85%, 90%, 95%, 96%, 97%, 98%, 99%, or 100% identical to the amino acid sequence of SEQ ID NO: 93.

In some embodiments of any of the aspects described herein, E (e.g., each E) includes the amino acid sequence of SEQ ID NO: 94. In some embodiments, E includes an amino acid sequence that is at least 70%, 75%, 80%, 85%, 90%, 95%, 96%, 97%, 98%, 99%, or 100% identical to the amino acid sequence of SEQ ID NO: 94.

In some embodiments of any of the aspects described herein, E (e.g., each E) includes the amino acid sequence of SEQ ID NO: 95. In some embodiments, E includes an amino acid sequence that is at least 70%, 75%, 80%, 85%, 90%, 95%, 96%, 97%, 98%, 99%, or 100% identical to the amino acid sequence of SEQ ID NO: 95.

In some embodiments of any of the aspects described herein, E (e.g., each E) includes the amino acid sequence of SEQ ID NO: 96. In some embodiments, E includes an amino acid sequence that is at least 70%, 75%, 80%, 85%, 90%, 95%, 96%, 97%, 98%, 99%, or 100% identical to the amino acid sequence of SEQ ID NO: 96.

In some embodiments of any of the aspects described herein, E (e.g., each E) includes the amino acid sequence of SEQ ID NO: 97. In some embodiments, E includes an amino acid sequence that is at least 70%, 75%, 80%, 85%, 90%, 95%, 96%, 97%, 98%, 99%, or 100% identical to the amino acid sequence of SEQ ID NO: 97.

In some embodiments of any of the aspects described herein, E (e.g., each E) includes the amino acid sequence of SEQ ID NO: 98. In some embodiments, E includes an amino acid sequence that is at least 70%, 75%, 80%, 85%, 90%, 95%, 96%, 97%, 98%, 99%, or 100% identical to the amino acid sequence of SEQ ID NO: 98.

In some embodiments of any of the aspects described herein, wherein E includes an Fc domain monomer, the Fc domain monomer (e.g., the Fc domain monomer having the sequence of any one of SEQ ID NOs: 1-95) includes a triple mutation corresponding to M252Y/S254T/T256E (YTE). As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-95 may be mutated to include a YTE mutation.

In some embodiments of any of the aspects described herein, wherein E includes an Fc domain monomer, the Fc domain monomer (e.g., the Fc domain monomer having the sequence of any one of SEQ ID NOs: 1-95) includes a double mutant corresponding to M428L/N434S (LS). As used herein, an amino acid “corresponding to” a particular amino acid residue (e.g., or a particular SEQ ID NO.) should be understood to include any amino acid residue that one of skill in the art would understand to align to the particular residue (e.g., of the particular sequence). For example, any one of SEQ ID NOs: 1-95 may be mutated to include a LS mutation.

In some embodiments of any of the aspects described herein, wherein E includes an Fc domain monomer, the Fc domain monomer (e.g., the Fc domain monomer having the sequence of any one of SEQ ID NOs: 1-95) includes a mutant corresponding to N434H.

In some embodiments of any of the aspects described herein, wherein E includes an Fc domain monomer, the Fc domain monomer (e.g., the Fc domain monomer having the sequence of any one of SEQ ID NOs: 1-95) includes a mutant corresponding to C220S. As used herein, an amino acid “corresponding to” a particular amino acid residue (e.g., or a particular SEQ ID NO.) should be understood to include any amino acid residue that one of skill in the art would understand to align to the particular residue (e.g., of the particular sequence). For example, any one of SEQ ID NOs: 1-95 may be mutated to include a C220S mutation.

As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-95 may be mutated to include an N434H mutation.

In some embodiments of any of the aspects described herein, wherein E includes an Fc domain monomer, the Fc domain monomer (e.g., the Fc domain monomer having the sequence of any one of SEQ ID NOs: 1-95) is a fragment of the Fc domain monomer (e.g., a fragment of at least 25 (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more), at least 50 (e.g., 51, 52, 53, 54, 55, 56, 57, 58, 59, 60, 61, 62, 63, 64, 65, 66, 67, 68, 69, 70, 71, 72, 73, 74, 75 or more), at least 75 (e.g., 75, 76, 77, 78, 79, 80, 81, 82, 83, 84, 85, 86, 87, 88, 89, 90, 91, 92, 93, 94, 95, 96, 97, 98, 99, 100 or more) consecutive amino acids in length from SEQ ID NOs: 1-95.

In some embodiments of any of the aspects described herein (e.g., a conjugate of any one of formulas (1), (2), (D-I)-(D-IV), or (M-I)-(M-IV)), one or more nitrogen atoms of one or more surface exposed lysine residues of E or one or more sulfur atoms of one or more surface exposed cysteines in E is covalently conjugated to a linker (e.g., a PEG₂-PEG₂₀ linker). The linker conjugated to E may be functionalized such that it may react to form a covalent bond with the L of any A₁-L or any A₂-L-A₁ described herein. In preferred embodiments, E is conjugated to a linker functionalized with an azido group and the L of A₁-L or any A₂-L-A₁ is functionalized with an alkyne group. Conjugation (e.g., by click chemistry) of the linker-azido of E and the linker-alkyne of A₁-L or A₂-L-A₁ forms a conjugate of the invention, for example a conjugate described by any one of formulas (1), (2), (D-I)-(D-IV), or (M-I)-(M-IV). In yet other embodiments, E is conjugated to a linker functionalized with an alkyne group and L of an A₁-L or of any A₂-L-A₁ is functionalized with an azido group. Conjugate (e.g., by click chemistry) of the linker-alkyne of E and linker-azido of A₁-L or of any A₂-L-A₁ forms a conjugate of the invention, for example a conjugate described by any one of formulas (1), (2), (D-I)-(D-IV), or (M-I)-(M-IV).

In some embodiments of any of the aspects described herein, the squiggly line of any one of formulas (1), (2), (D-I)-(D-IV), or (M-I)-(M-IV) represents a covalent bond between the L of A₁-L or A₂-L-A₁.

In some embodiments of any of the aspects described herein, the squiggly line of any one of formulas (1), (2), (D-I)-(D-IV), or (M-I)-(M-IV) represents that one or more amino acid side chains of E (e.g., one or more nitrogen atoms of one or more surface exposed lysine residues of E or one or more sulfur atoms of one or more surface exposed cysteines in E) have been conjugated to a linker (e.g., a PEG₂-PEG₂₀ linker) wherein the linker has been functionalized with a reactive moiety, such that the reactive moiety forms a covalent bond with the L of any A₁-L or any A₂-L-A₁ described herein (e.g., by click chemistry between an azido functionalized linker and an alkyne functionalized linker, as described above).

In some embodiments of any of the aspects described herein, each A₁ and/or A₂ have the structure described by (A-I):

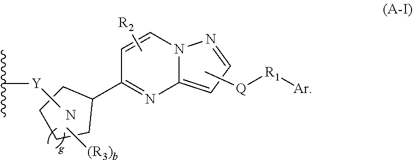

In preferred embodiments, each A₁ and/or A₂ have the structure described by:

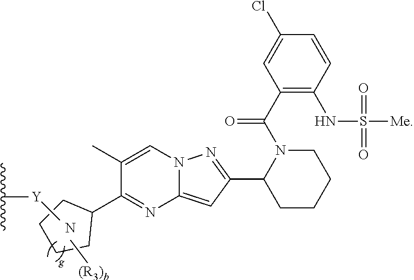

In some embodiments of any of the aspects described herein, each A₁ and/or A₂ have the structure described by (A-II):

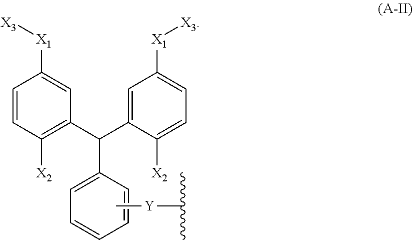

In preferred embodiments, each A₁ and/or A₂ have the structure described by:

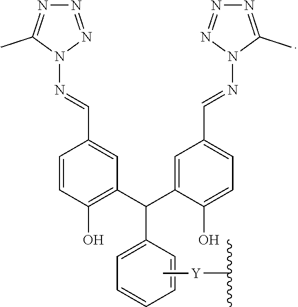

In some embodiments of any of the aspects described herein, each A₁ and/or A₂ have the structure described by (A-III):

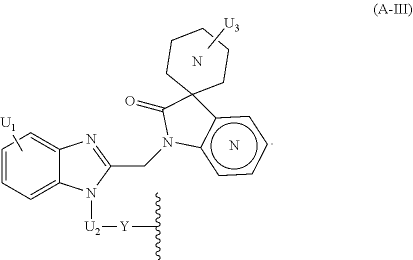

In preferred embodiments, each A₁ and/or A₂ have the structure described by:

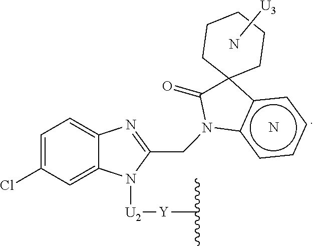

In some embodiments, U₂ is C₁-C₆ alkyl.

In some embodiments, the conjugate is conjugate 1, or any regioisomer thereof, and the drug-to-antibody ratio (DAR) (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 2, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 3, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 4, or any regioisomer thereof, and the drug-to-antibody ratio (DAR) (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 5, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 6, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 7, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 8, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 9, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 11, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 11, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 12, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 13, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 14, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 15, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 16, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 17, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 18, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, the conjugate is conjugate 19, or any regioisomer thereof, and the DAR (e.g., T) is between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In some embodiments the DAR is between 0.5 and 2.0, between 2.0 and 4.0, between 4.0 and 6.0 between 6.0 and 8.0, or between 8.0 and 10.0.

In some embodiments, a population of conjugates described herein has a DAR (e.g., T) of between 1 and 2, 2 and 4, 4 and 6, 6 and 8, 8 and 10, 1 and 10, 1 and 20, 1 and 5, 3 and 7, 5 and 10, or 10 and 20.

In some embodiments, the Fc domain monomer includes less than about 300 amino acid residues (e.g., less than about 300, less than about 295, less than about 290, less than about 285, less than about 280, less than about 275, less than about 270, less than about 265, less than about 260, less than about 255, less than about 250, less than about 245, less than about 240, less than about 235, less than about 230, less than about 225, or less than about 220 amino acid residues). In some embodiments, the Fc domain monomer is less than about 40 kDa (e.g., less than about 35 kDa, less than about 30 kDa, less than about 25 kDa).

In some embodiments, the Fc domain monomer includes at least 200 amino acid residues (e.g., at least 210, at least 220, at least 230, at least 240, at least 250, at least 260, at least 270, at least 280, at least 290, or at least 300 amino residues). In some embodiments, the Fc domain monomer is at least 20 kDa (e.g., at least 25 kDa, at least 30 kDa, or at least 35 kDa).

In some embodiments, the Fc domain monomer includes 200 to 400 amino acid residues (e.g., 200 to 250, 250 to 300, 300 to 350, 350 to 400, 200 to 300, 250 to 350, or 300 to 400 amino acid residues). In some embodiments, the Fc domain monomer is 20 to 40 kDa (e.g., 20 to 25 kDa, 25 to 30 kDa, 35 to 40 kDa, 20 to 30 kDa, 25 to 35 kDa, or 30 to 40 KDa).

In some embodiments, the Fc domain monomer includes an amino acid sequence at least 90% identical (e.g., at least 95%, at least 98%) to the sequence of any one of SEQ ID NOs: 1-95, or a region thereof. In some embodiments, the Fc domain monomer includes the amino acid sequence of any one of SEQ ID NOs: 1-95, or a region thereof.

In some embodiments, the Fc domain monomer includes a region of any one of SEQ ID NOs: 1-95, wherein the region includes positions 220, 252, 254, and 256. In some embodiments, the region includes at least 40 amino acid residues, at least 50 amino acid residues, at least 60 amino acid residues, at least 70 amino acids residues, at least 80 amino acids residues, at least 90 amino acid residues, at least 100 amino acid residues, at least 110 amino acid residues, at least 120 amino residues, at least 130 amino acid residues, at least 140 amino acid residues, at least 150 amino acid residues, at least 160 amino acid residues, at least 170 amino acid residues, at least 180 amino acid residues, at least 190 amino acid residues, or at least 200 amino acid residues.

### Definitions

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

By “viral infection” is meant the pathogenic growth of a virus (e.g., RSV such as RSV A or RSV B) in a host organism (e.g., a human subject). A viral infection can be any situation in which the presence of a viral population(s) is damaging to a host body. Thus, a subject is “suffering” from a viral infection when an excessive amount of a viral population is present in or on the subject's body, or when the presence of a viral population(s) is damaging the cells or other tissue of the subject.

As used herein, the term “Fc domain monomer” refers to a polypeptide chain that includes at least a hinge domain and second and third antibody constant domains (C_{H}2 and C_{H}3) or functional fragments thereof (e.g., fragments that that capable of (i) dimerizing with another Fc domain monomer to form an Fc domain, and (ii) binding to an Fc receptor. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., IgG1). An Fc domain monomer does not include any portion of an immunoglobulin that is capable of acting as an antigen-recognition region, e.g., a variable domain or a complementarity determining region (CDR). Fc domain monomers in the conjugates as described herein can contain one or more changes from a wild-type Fc domain monomer sequence (e.g., 1-10, 1-8, 1-6, 1-4 amino acid substitutions, additions, or deletions) that alter the interaction between an Fc domain and an Fc receptor. Examples of suitable changes are known in the art. In certain embodiments, a human Fc domain monomer (e.g., an IgG heavy chain, such as IgG1) includes a region that extends from any of Asn208, Glu216, Asp221, Lys222, or Cys226 to the carboxyl-terminus of the heavy chain at Lys447. C-terminal Lys447 of the Fc region may or may not be present, without affecting the structure or stability of the Fc region. C-terminal Lys 447 may be proteolytically cleaved upon expression of the polypeptide. In some embodiments of any of the Fc domain monomers described herein, C-terminal Lys 447 is optionally present or absent. The disclosure specifically contemplates any of SEQ ID NOs: 1-4, 11, 16, 19, 20, 32-37, 48-53, and 60-68 that do not include the C-terminal Lys corresponding to Lys447. The N-terminal N (Asn) of the Fc region (e.g., of any one of SEQ ID NOs: 60-77) may or may not be present, without affecting the structure of stability of the Fc region. N-terminal Asn may be deamidated upon expression of the polypeptide. In some embodiments of any of the Fc domain monomers described herein, N-terminal Asn is optionally present or absent. The disclosure specifically contemplates any of SEQ ID NOs: 60-77 that do not include the N-terminal Asn. Unless otherwise specified herein, numbering of amino acid residues in the IgG or Fc domain monomer is according to the EU numbering system for antibodies, also called the Kabat EU index, as described, for example, in Kabat et al., Sequences of Proteins of Immunological Interest, 5th Ed. Public Health Service, National Institutes of Health, Bethesda, Md., 1991.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, in some embodiments, one or more disulfide bonds form between the hinge domains of the two dimerizing Fc domain monomers.

The term “covalently attached” refers to two parts of a conjugate that are linked to each other by a covalent bond formed between two atoms in the two parts of the conjugate.

As used herein, the term “Fc-binding peptide” refers to refers to a polypeptide having an amino acid sequence of 5 to 50 (e.g., 5 to 40, 5 to 30, 5 to 20, 5 to 15, 5 to 10, 10 to 50, 10 to 30, or 10 to 20) amino acid residues that has affinity for and functions to bind an Fc domain, such as any of the Fc domain described herein. An Fc-binding peptide can be of different origins, e.g., synthetic, human, mouse, or rat. Fc-binding peptides of the invention include Fc-binding peptides which have been engineered to include one or more (e.g., two, three, four, or five) solvent-exposed cysteine or lysine residues, which may provide a site for conjugation to a compound of the invention (e.g., conjugation to a RSV F protein inhibitor monomer or dimer, including by way of a linker). Most preferably, the Fc-binding peptide will contain a single solvent-exposed cysteine or lysine, thus enabling site-specific conjugation of a compound of the invention. Fc-binding peptides may include only naturally occurring amino acid residues, or may include one or more non-naturally occurring amino acid residues. Where included, a non-naturally occurring amino acid residue (e.g., the side chain of a non-naturally occurring amino acid residue) may be used as the point of attachment for a compound of the invention (e.g., a RSV F protein inhibitor monomer or dimer, including by way of a linker). Fc-binding peptides of the invention may be linear or cyclic. Fc-binding peptides of the invention include any Fc-binding peptides known to one of skill in the art.

As used here, the term “albumin protein” refers to a polypeptide including an amino acid sequence corresponding to a naturally-occurring albumin protein (e.g., human serum albumin) or a variant thereof, such as an engineered variant of a naturally-occurring albumin protein. Variants of albumin proteins include polymorphisms, fragments such as domains and sub-domains, and fusion proteins (e.g., an albumin protein having a C-terminal or N-terminal fusion, such as a polypeptide linker). Preferably the albumin protein has the amino acid sequence of human serum albumin (HSA) or a variant or fragment thereof, most preferably a functional variant or fragment thereof. Albumin proteins of the invention include proteins having at least 70%, 75%, 80%, 85%, 90%, 91%, 92%, 93%, 94%, 95%, 96%, 97%, 98%, or 99% identity to any one of SEQ ID NOs: 96-98. Albumin proteins of the invention include albumin proteins which have been engineered to include one or more (e.g., two, three, four, or five) solvent-exposed cysteine or lysine residues, which may provide a site for conjugation to a compound of the invention (e.g., conjugation to a RSV F protein inhibitor monomer or dimer, including by way of a linker). Most preferably, the albumin protein will contain a single solvent-exposed cysteine or lysine, thus enabling site-specific conjugation of a compound of the invention. Albumin proteins may include only naturally occurring amino acid residues, or may include one or more non-naturally occurring amino acid residues. Where included, a non-naturally occurring amino acid residue (e.g., the side chain of a non-naturally occurring amino acid residue) may be used as the point of attachment for a compound of the invention (e.g., a RSV F protein inhibitor monomer or dimer, including by way of a linker).

As used herein, the term “albumin protein-binding peptide” refers to a polypeptide having an amino acid sequence of 5 to 50 (e.g., 5 to 40, 5 to 30, 5 to 20, 5 to 15, 5 to 10, 10 to 50, 10 to 30, or 10 to 20) amino acid residues that has affinity for and functions to bind an albumin protein, such as any of the albumin proteins described herein. Preferably, the albumin protein-binding peptide binds to a naturally-occurring serum albumin, most preferably human serum albumin. An albumin protein-binding peptide can be of different origins, e.g., synthetic, human, mouse, or rat. Albumin protein-binding peptides of the invention include albumin protein-binding peptides which have been engineered to include one or more (e.g., two, three, four, or five) solvent-exposed cysteine or lysine residues, which may provide a site for conjugation to a compound of the invention (e.g., conjugation to a RSV F protein inhibitor monomer or dimer, including by way of a linker). Most preferably, the albumin protein-binding peptide will contain a single solvent-exposed cysteine or lysine, thus enabling site-specific conjugation of a compound of the invention. Albumin protein-binding peptides may include only naturally occurring amino acid residues, or may include one or more non-naturally occurring amino acid residues. Where included, a non-naturally occurring amino acid residue (e.g., the side chain of a non-naturally occurring amino acid residue) may be used as the point of attachment for a compound of the invention (e.g., a RSV F protein inhibitor monomer or dimer, including by way of a linker). Albumin protein-binding peptides of the invention may be linear or cyclic. Albumin protein-binding peptide of the invention include any albumin protein-binding peptides known to one of skill in the art, examples of which, are provided herein. Further exemplary albumin protein-binding peptides are provided in U.S. Patent Application No. 2005/0287153, which is incorporated herein by reference in its entirety.

As used-herein, a “surface exposed amino acid” or “solvent-exposed amino acid,” such as a surface exposed cysteine or a surface exposed lysine refers to an amino acid that is accessible to the solvent surrounding the protein. A surface exposed amino acid may be a naturally-occurring or an engineered variant (e.g., a substitution or insertion) of the protein. In some embodiments, a surface exposed amino acid is an amino acid that when substituted does not substantially change the three-dimensional structure of the protein.

The terms “linker,” “L,” and “L′,” as used herein, refer to a covalent linkage or connection between two or more components in a conjugate (e.g., between two RSV F protein inhibitors in a conjugate described herein, between a RSV F protein inhibitor and an Fc domain or albumin protein in a conjugate described herein, and between a dimer of two RSV F protein inhibitors and an Fc domain or an albumin protein in a conjugate described herein). In some embodiments, a conjugate described herein may contain a linker that has a trivalent structure (e.g., a trivalent linker). A trivalent linker has three arms, in which each arm is covalently linked to a component of the conjugate (e.g., a first arm conjugated to a first RSV F protein inhibitor, a second arm conjugated to a second RSV F protein inhibitor, and a third arm conjugated to an Fc domain or an albumin protein).

Molecules that may be used as linkers include at least two functional groups, which may be the same or different, e.g., two carboxylic acid groups, two amine groups, two sulfonic acid groups, a carboxylic acid group and a maleimide group, a carboxylic acid group and an alkyne group, a carboxylic acid group and an amine group, a carboxylic acid group and a sulfonic acid group, an amine group and a maleimide group, an amine group and an alkyne group, or an amine group and a sulfonic acid group. The first functional group may form a covalent linkage with a first component in the conjugate and the second functional group may form a covalent linkage with the second component in the conjugate. In some embodiments of a trivalent linker, two arms of a linker may contain two dicarboxylic acids, in which the first carboxylic acid may form a covalent linkage with the first RSV F protein inhibitor in the conjugate and the second carboxylic acid may form a covalent linkage with the second RSV F protein inhibitor in the conjugate, and the third arm of the linker may for a covalent linkage with an Fc domain or albumin protein in the conjugate. Examples of dicarboxylic acids are described further herein. In some embodiments, a molecule containing one or more maleimide groups may be used as a linker, in which the maleimide group may form a carbon-sulfur linkage with a cysteine in a component (e.g., an Fc domain or an albumin protein) in the conjugate. In some embodiments, a molecule containing one or more alkyne groups may be used as a linker, in which the alkyne group may form a 1,2,3-triazole linkage with an azide in a component (e.g., an Fc domain or an albumin protein) in the conjugate. In some embodiments, a molecule containing one or more azide groups may be used as a linker, in which the azide group may form a 1,2,3-triazole linkage with an alkyne in a component (e.g., an Fc domain or an albumin protein) in the conjugate. In some embodiments, a molecule containing one or more bis-sulfone groups may be used as a linker, in which the bis-sulfone group may form a linkage with an amine group a component (e.g., an Fc domain or an albumin protein) in the conjugate. In some embodiments, a molecule containing one or more sulfonic acid groups may be used as a linker, in which the sulfonic acid group may form a sulfonamide linkage with a component in the conjugate. In some embodiments, a molecule containing one or more isocyanate groups may be used as a linker, in which the isocyanate group may form a urea linkage with a component in the conjugate. In some embodiments, a molecule containing one or more haloalkyl groups may be used as a linker, in which the haloalkyl group may form a covalent linkage, e.g., C—N and C—O linkages, with a component in the conjugate.

In some embodiments, a linker provides space, rigidity, and/or flexibility between the two or more components. In some embodiments, a linker may be a bond, e.g., a covalent bond. The term “bond” refers to a chemical bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms. In some embodiments, a linker includes no more than 250 non-hydrogen atoms. In some embodiments, the backbone of a linker includes no more than 250 atoms. The “backbone” of a linker refers to the atoms in the linker that together form the shortest path from one part of a conjugate to another part of the conjugate (e.g., the shortest path linking a first RSV F protein inhibitor and a second RSV F protein inhibitor). The atoms in the backbone of the linker are directly involved in linking one part of a conjugate to another part of the conjugate (e.g., linking a first RSV F protein inhibitor and a second RSV F protein inhibitor). For examples, hydrogen atoms attached to carbons in the backbone of the linker are not considered as directly involved in linking one part of the conjugate to another part of the conjugate.

In some embodiments, a linker may comprise a synthetic group derived from, e.g., a synthetic polymer (e.g., a polyethylene glycol (PEG) polymer). In some embodiments, a linker may comprise one or more amino acid residues, such as D- or L-amino acid residues. In some embodiments, a linker may be a residue of an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may comprise one or more, e.g., 1-100, 1-50, 1-25, 1-10, 1-5, or 1-3, optionally substituted alkylene, optionally substituted heteroalkylene (e.g., a PEG unit), optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted cycloalkenylene, optionally substituted heterocycloalkenylene, optionally substituted cycloalkynylene, optionally substituted heterocycloalkynylene, optionally substituted arylene, optionally substituted heteroarylene (e.g., pyridine), O, S, NRⁱ (Rⁱ is H, optionally substituted alkyl, optionally substituted heteroalkyl, optionally substituted alkenyl, optionally substituted heteroalkenyl, optionally substituted alkynyl, optionally substituted heteroalkynyl, optionally substituted cycloalkyl, optionally substituted heterocycloalkyl, optionally substituted cycloalkenyl, optionally substituted heterocycloalkenyl, optionally substituted cycloalkynyl, optionally substituted heterocycloalkynyl, optionally substituted aryl, or optionally substituted heteroaryl), P, carbonyl, thiocarbonyl, sulfonyl, phosphate, phosphoryl, or imino. For example, a linker may comprise one or more optionally substituted C₁-C₂₀ alkylene, optionally substituted C₁-C₂₀ heteroalkylene (e.g., a PEG unit), optionally substituted C₂-C₂₀ alkenylene (e.g., C₂ alkenylene), optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene (e.g., cyclopropylene, cyclobutylene), optionally substituted C₂-C₂₀ heterocycloalkylene, optionally substituted C₄-C₂₀ cycloalkenylene, optionally substituted C₄-C₂₀ heterocycloalkenylene, optionally substituted C₅-C₂₀ cycloalkynylene, optionally substituted C₅-C₂₀ heterocycloalkynylene, optionally substituted C₅-C₁₅ arylene (e.g., C₆ arylene), optionally substituted C₃-C₁₅ heteroarylene (e.g., imidazole, pyridine), O, S, NRⁱ (Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₅-C₂₀ cycloalkynyl, optionally substituted C₈-C₂₀ heterocycloalkynyl, optionally substituted C₅-C₁₅ aryl, or optionally substituted C₃-C₁₅ heteroaryl), P, carbonyl, thiocarbonyl, sulfonyl, phosphate, phosphoryl, or imino.

The terms “alkyl,” “alkenyl,” and “alkynyl,” as used herein, include straight-chain and branched-chain monovalent substituents, as well as combinations of these, containing only C and H when unsubstituted. When the alkyl group includes at least one carbon-carbon double bond or carbon-carbon triple bond, the alkyl group can be referred to as an “alkenyl” or “alkynyl” group respectively. The monovalency of an alkyl, alkenyl, or alkynyl group does not include the optional substituents on the alkyl, alkenyl, or alkynyl group. For example, if an alkyl, alkenyl, or alkynyl group is attached to a compound, monovalency of the alkyl, alkenyl, or alkynyl group refers to its attachment to the compound and does not include any additional substituents that may be present on the alkyl, alkenyl, or alkynyl group. In some embodiments, the alkyl or heteroalkyl group may contain, e.g., 1-20. 1-18, 1-16, 1-14, 1-12, 1-10, 1-8, 1-6, 1-4, or 1-2 carbon atoms (e.g., C₁-C₂₀, C₁-C₁₈, C₁-C₁₆, C₁-C₁₄, C₁-C₁₂, C₁-C₁₀, C₁-C₈, C₁-C₆, C₁-C₄, or C₁-C₂). In some embodiments, the alkenyl, heteroalkenyl, alkynyl, or heteroalkynyl group may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Examples include, but are not limited to, methyl, ethyl, isobutyl, sec-butyl, tert-butyl, 2-propenyl, and 3-butynyl.

The term “cycloalkyl,” as used herein, represents a monovalent saturated or unsaturated non-aromatic cyclic alkyl group. A cycloalkyl may have, e.g., three to twenty carbons (e.g., a C₃-C₇, C₃-C₈, C₃-C₉, C₃-C₁₀, C₃-C₁₁, C₃-C₁₂, C₃-C₁₄, C₃-C₁₆, C₃-C₁₈, or C₃-C₂₀ cycloalkyl). Examples of cycloalkyls include, but are not limited to, cyclopropyl, cyclobutyl, cyclopentyl, cyclohexyl, and cycloheptyl. When the cycloalkyl group includes at least one carbon-carbon double bond, the cycloalkyl group can be referred to as a “cycloalkenyl” group. A cycloalkenyl may have, e.g., four to twenty carbons (e.g., a C₄-C₇, C₄-C₈, C₄-C₉, C₄-C₁₀, C₄-C₁₁, C₄-C₁₂, C₄-C₁₄, C₄-C₁₆, C₄-C₁₈, or C₄-C₂₀ cycloalkenyl). Exemplary cycloalkenyl groups include, but are not limited to, cyclopentenyl, cyclohexenyl, and cycloheptenyl. When the cycloalkyl group includes at least one carbon-carbon triple bond, the cycloalkyl group can be referred to as a “cycloalkynyl” group. A cycloalkynyl may have, e.g., eight to twenty carbons (e.g., a C₈-C₉, C₈-C₁₀, C₈-C₁₂, C₈-C₁₄, C₈-C₁₆, C₈-C₁₈, or C₈-C₂₀ cycloalkynyl). The term “cycloalkyl” also includes a cyclic compound having a bridged multicyclic structure in which one or more carbons bridges two non-adjacent members of a monocyclic ring, e.g., bicyclo[2.2.1]heptyl and adamantane. The term “cycloalkyl” also includes bicyclic, tricyclic, and tetracyclic fused ring structures, e.g., decalin and spiro cyclic compounds. A “heterocycloalkyl,” “heterocycloalkenyl,” or “heterocycloalkynyl” group refers to a cycloalkyl, cycloalkenyl, or cycloalkynyl group having one or more rings (e.g., 1, 2, 3, 4 or more rings) that has one or more heteroatoms independently selected from, e.g., N, O, and S. Exemplary heterocycloalkyl groups include pyrrolidine, thiophene, thiolane, tetrahydrofuran, piperidine, tetrahydropyran, quinoline, isoquinoline, cinnoline, phthalazine, quinazoline, quinoxaline, indole, benzothiophene, benzofuran, isoindole, benzo[c]thiophene, isobenzofuran, benzimidazole, benzoxazole, benzothiazole, 1H-indazole, 1,2,benzisoxazole, 1,2-benzisothiazole, 2,1-benzisothiazole, 2,1-benzisoxazole, purine, pyrrolizidine, indene, fluorene, carbazole, dibenzofuran, acridine, phenazine, and phenoxazine.

The term “aryl,” as used herein, refers to any monocyclic or fused ring bicyclic or tricyclic system which has the characteristics of aromaticity in terms of electron distribution throughout the ring system, e.g., phenyl, naphthyl, or phenanthrene. In some embodiments, a ring system contains 5-15 ring member atoms or 5-10 ring member atoms. An aryl group may have, e.g., five to fifteen carbons (e.g., a C₅-C₆, C₅-C₇, C₅-C₈, C₅-C₉, C₅-C₁₀, C₅-C₁₁, C₅-C₁₂, C₅-C₁₃, C₅-C₁₄, or C₅-C₁₅ aryl). The term “heteroaryl” also refers to such monocyclic or fused bicyclic ring systems containing one or more, e.g., 1-4, 1-3, 1, 2, 3, or 4, heteroatoms selected from O, S and N. A heteroaryl group may have, e.g., two to fifteen ring member atoms (e.g., a C₂-C₃, C₂-C₄, C₂-C₅, C₂-C₆, C₂-C₇, C₂-C₈, C₂-C₉, C₂-C₁₀, C₂-C₁₁, C₂-C₁₂, C₂-C₁₃, C₂-C₁₄, or C₃-C₁₅ heteroaryl). The inclusion of a heteroatom permits inclusion of 5-membered rings to be considered aromatic as well as 6-membered rings. Thus, typical heteroaryl systems include, e.g., pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, triazolyl (e.g., 1,2,3- or 1,2,4-triazolyl) oxazolyl, isoxazolyl, benzoxazolyl, benzoisoxazolyl, and imidazolyl. Because tautomers are possible, a group such as phthalimido is also considered heteroaryl. In some embodiments, the aryl or heteroaryl group is a 5- or 6-membered aromatic rings system optionally containing 1-2 nitrogen atoms. In some embodiments, the aryl or heteroaryl group is an optionally substituted phenyl, pyridyl, indolyl, pyrimidyl, pyridazinyl, benzothiazolyl, benzimidazolyl, pyrazolyl, imidazolyl, isoxazolyl, thiazolyl, or imidazopyridinyl. In some embodiments, the aryl group is phenyl. In some embodiments, an aryl group may be optionally substituted with a substituent such an aryl substituent, e.g., biphenyl.

The term “alkaryl,” refers to an aryl group that is connected to an alkylene, alkenylene, or alkynylene group. In general, if a compound is attached to an alkaryl group, the alkylene, alkenylene, or alkynylene portion of the alkaryl is attached to the compound. In some embodiments, an alkaryl is C₆-C₃₅ alkaryl (e.g., C₆-C₁₆, C₆-C₁₄, C₆-C₁₂, C₆-C₁₀, C₆-C₉, C₆-C₈, C₇, or C₆ alkaryl), in which the number of carbons indicates the total number of carbons in both the aryl portion and the alkylene, alkenylene, or alkynylene portion of the alkaryl. Examples of alkaryls include, but are not limited to, (C₁-C₈)alkylene(C₆-C₁₂)aryl, (C₂-C₈)alkenylene(C₆-C₁₂)aryl, or (C₂-C₈)alkynylene(C₆-C₁₂)aryl. In some embodiments, an alkaryl is benzyl or phenethyl. In a heteroalkaryl, one or more heteroatoms selected from N, O, and S may be present in the alkylene, alkenylene, or alkynylene portion of the alkaryl group and/or may be present in the aryl portion of the alkaryl group. In an optionally substituted alkaryl, the substituent may be present on the alkylene, alkenylene, or alkynylene portion of the alkaryl group and/or may be present on the aryl portion of the alkaryl group.

The term “amino,” as used herein, represents —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino group is —NH₂.

The term “alkamino,” as used herein, refers to an amino group, described herein, that is attached to an alkylene (e.g., C1-C5 alkylene), alkenylene (e.g., C2-C5 alkenylene), or alkynylene group (e.g., C2-C5 alkenylene). In general, if a compound is attached to an alkamino group, the alkylene, alkenylene, or alkynylene portion of the alkamino is attached to the compound. The amino portion of an alkamino refers to —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino portion of an alkamino is —NH₂. An example of an alkamino group is C1-C5 alkamino, e.g., C2 alkamino (e.g., CH₂CH₂NH₂ or CH₂CH₂N(CH₃)₂). In a heteroalkamino group, one or more, e.g., 1-4, 1-3, 1, 2, 3, or 4, heteroatoms selected from N, O, and S may be present in the alkylene, alkenylene, or alkynylene portion of the heteroalkamino group. In some embodiments, an alkamino group may be optionally substituted. In a substituted alkamino group, the substituent may be present on the alkylene, alkenylene, or alkynylene portion of the alkamino group and/or may be present on the amino portion of the alkamino group.

The term “alkamide,” as used herein, refers to an amide group that is attached to an alkylene (e.g., C1-C5 alkylene), alkenylene (e.g., C2-C5 alkenylene), or alkynylene (e.g., C2-C5 alkenylene) group. In general, if a compound is attached to an alkamide group, the alkylene, alkenylene, or alkynylene portion of the alkamide is attached to the compound. The amide portion of an alkamide refers to —C(O)—N(R^{x})₂, where each R^{x} is, independently, H, alkyl, alkenyl, alkynyl, aryl, alkaryl, cycloalkyl, or two R^{x} combine to form a heterocycloalkyl. In some embodiment, the amide portion of an alkamide is —C(O)NH₂. An alkamide group may be —(CH₂)₂—C(O)NH₂ or —CH₂—C(O)NH₂. In a heteroalkamide group, one or more, e.g., 1-4, 1-3, 1, 2, 3, or 4, heteroatoms selected from N, O, and S may be present in the alkylene, alkenylene, or alkynylene portion of the heteroalkamide group. In some embodiments, an alkamide group may be optionally substituted. In a substituted alkamide group, the substituent may be present on the alkylene, alkenylene, or alkynylene portion of the alkamide group and/or may be present on the amide portion of the alkamide group.

The terms “alkylene,” “alkenylene,” and “alkynylene,” as used herein, refer to divalent groups having a specified size. In some embodiments, an alkylene may contain, e.g., 1-20, 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, an alkenylene or alkynylene may contain, e.g., 2-20, 2-18, 2-16, 2-14, 2-12, 2-10, 2-8, 2-6, or 2-4 carbon atoms (e.g., C2-C20, C2-C18, C2-C16, C2-C14, C2-C12, C2-C10, C2-C8, C2-C6, or C2-C4). Alkylene, alkenylene, and/or alkynylene includes straight-chain and branched-chain forms, as well as combinations of these. The divalency of an alkylene, alkenylene, or alkynylene group does not include the optional substituents on the alkylene, alkenylene, or alkynylene group. For example, two RSV F protein inhibitors may be attached to each other by way of a linker that includes alkylene, alkenylene, and/or alkynylene, or combinations thereof. Each of the alkylene, alkenylene, and/or alkynylene groups in the linker is considered divalent with respect to the two attachments on either end of alkylene, alkenylene, and/or alkynylene group. For example, if a linker includes -(optionally substituted alkylene)-(optionally substituted alkenylene)-(optionally substituted alkylene)-, the alkenylene is considered divalent with respect to its attachments to the two alkylenes at the ends of the linker. The optional substituents on the alkenylene are not included in the divalency of the alkenylene. The divalent nature of an alkylene, alkenylene, or alkynylene group (e.g., an alkylene, alkenylene, or alkynylene group in a linker) refers to both of the ends of the group and does not include optional substituents that may be present in an alkylene, alkenylene, or alkynylene group. Because they are divalent, they can link together multiple (e.g., two) parts of a conjugate, e.g., a first RSV F protein inhibitor and a second RSV F protein inhibitor. Alkylene, alkenylene, and/or alkynylene groups can be substituted by the groups typically suitable as substituents for alkyl, alkenyl and alkynyl groups as set forth herein. For example, C═O is a C1 alkylene that is substituted by an oxo (═O). For example, —HCR—C≡C— may be considered as an optionally substituted alkynylene and is considered a divalent group even though it has an optional substituent, R. Heteroalkylene, heteroalkenylene, and/or heteroalkynylene groups refer to alkylene, alkenylene, and/or alkynylene groups including one or more, e.g., 1-4, 1-3, 1, 2, 3, or 4, heteroatoms, e.g., N, O, and S. For example, a polyethylene glycol (PEG) polymer or a PEG unit —(CH₂)₂—O— in a PEG polymer is considered a heteroalkylene containing one or more oxygen atoms.

The term “cycloalkylene,” as used herein, refers to a divalent cyclic group linking together two parts of a compound. For example, one carbon within the cycloalkylene group may be linked to one part of the compound, while another carbon within the cycloalkylene group may be linked to another part of the compound. A cycloalkylene group may include saturated or unsaturated non-aromatic cyclic groups. A cycloalkylene may have, e.g., three to twenty carbons in the cyclic portion of the cycloalkylene (e.g., a C3-C7, C3-C8, C3-C9, C3-C10, C3-C11, C3-C12, C3-C14, C3-C16, C3-C18, or C3-C20 cycloalkylene). When the cycloalkylene group includes at least one carbon-carbon double bond, the cycloalkylene group can be referred to as a “cycloalkenylene” group. A cycloalkenylene may have, e.g., four to twenty carbons in the cyclic portion of the cycloalkenylene (e.g., a C4-C7, C4-C8, C4-C9. C4-C10, C4-C11, C4-C12, C4-C14, C4-C16, C4-C18, or C4-C20 cycloalkenylene). When the cycloalkylene group includes at least one carbon-carbon triple bond, the cycloalkylene group can be referred to as a “cycloalkynylene” group. A cycloalkynylene may have, e.g., four to twenty carbons in the cyclic portion of the cycloalkynylene (e.g., a C4-C7, C4-C8, C4-C9. C4-C10, C4-C11, C4-C12, C4-C14, C4-C16, C4-C18, or C8-C20 cycloalkynylene). A cycloalkylene group can be substituted by the groups typically suitable as substituents for alkyl, alkenyl and alkynyl groups as set forth herein. Heterocycloalkylene refers to a cycloalkylene group including one or more, e.g., 1-4, 1-3, 1, 2, 3, or 4, heteroatoms, e.g., N, O, and S. Examples of cycloalkylenes include, but are not limited to, cyclopropylene and cyclobutylene. A tetrahydrofuran may be considered as a heterocycloalkylene.

The term “arylene,” as used herein, refers to a multivalent (e.g., divalent or trivalent) aryl group linking together multiple (e.g., two or three) parts of a compound. For example, one carbon within the arylene group may be linked to one part of the compound, while another carbon within the arylene group may be linked to another part of the compound. An arylene may have, e.g., five to fifteen carbons in the aryl portion of the arylene (e.g., a C5-C6, C5-C7, C5-C8, C5-C9. C5-C10, C5-C11, C5-C12, C5-C13, C5-C14, or C5-C15 arylene). An arylene group can be substituted by the groups typically suitable as substituents for alkyl, alkenyl and alkynyl groups as set forth herein. Heteroarylene refers to an aromatic group including one or more, e.g., 1-4, 1-3, 1, 2, 3, or 4, heteroatoms, e.g., N, O, and S. A heteroarylene group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroarylene).

The term “optionally substituted,” as used herein, refers to having 0, 1, or more substituents, such as 0-25, 0-20, 0-10 or 0-5 substituents. Substituents include, but are not limited to, alkyl, alkenyl, alkynyl, aryl, alkaryl, acyl, heteroaryl, heteroalkyl, heteroalkenyl, heteroalkynyl, heteroalkaryl, halogen, oxo, cyano, nitro, amino, alkamino, hydroxy, alkoxy, alkanoyl, carbonyl, carbamoyl, guanidinyl, ureido, amidinyl, any of the groups or moieties described above, and hetero versions of any of the groups or moieties described above. Substituents include, but are not limited to, F, Cl, methyl, phenyl, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, alkenyl, aryl, heteroalkyl, heteroalkenyl, or heteroaryl, and wherein two of the optional substituents on the same or adjacent atoms can be joined to form a fused, optionally substituted aromatic or nonaromatic, saturated or unsaturated ring which contains 3-8 members, or two of the optional substituents on the same atom can be joined to form an optionally substituted aromatic or nonaromatic, saturated or unsaturated ring which contains 3-8 members.

An optionally substituted group or moiety refers to a group or moiety (e.g., any one of the groups or moieties described above) in which one of the atoms (e.g., a hydrogen atom) is optionally replaced with another substituent. For example, an optionally substituted alkyl may be an optionally substituted methyl, in which a hydrogen atom of the methyl group is replaced by, e.g., OH. As another example, a substituent on a heteroalkyl or its divalent counterpart, heteroalkylene, may replace a hydrogen on a carbon or a hydrogen on a heteroatom such as N. For example, the hydrogen atom in the group —R—NH—R— may be substituted with an alkamide substituent, e.g., —R—N[(CH₂C(O)N(CH₃)₂]—R.

Generally, an optional substituent is a noninterfering substituent. A “noninterfering substituent” refers to a substituent that leaves the ability of the conjugates described herein (e.g., conjugates of any one of formulas (1), (2), (D-I)-(D-IV), or (M-I)-(M-IV)) to either bind to RSV F protein or to inhibit the proliferation of RSV. Thus, in some embodiments, the substituent may alter the degree of such activity. However, as long as the conjugate retains the ability to bind to RSV F protein or to inhibit RSV proliferation, the substituent will be classified as “noninterfering.” For example, the noninterfering substituent would leave the ability of the compound to provide antiviral efficacy based on an IC50 value of 10 μM or less in a viral plaque reduction assay. Thus, the substituent may alter the degree of inhibition based on plaque reduction or RSV F protein inhibition. However, as long as the compounds herein such as compounds of formulas (A-I), (A-II), and (A-III) retain the ability to inhibit RSV F protein, the substituent will be classified as “non interfering.” A number of assays for determining viral plaque reduction or the ability of any compound to inhibit RSV F protein are available in the art, and some are exemplified in the Examples below.

The term “hetero,” when used to describe a chemical group or moiety, refers to having at least one heteroatom that is not a carbon or a hydrogen, e.g., N, O, and S. Any one of the groups or moieties described above may be referred to as hetero if it contains at least one heteroatom. For example, a heterocycloalkyl, heterocycloalkenyl, or heterocycloalkynyl group refers to a cycloalkyl, cycloalkenyl, or cycloalkynyl group that has one or more heteroatoms independently selected from, e.g., N, O, and S. An example of a heterocycloalkenyl group is a maleimido. For example, a heteroaryl group refers to an aromatic group that has one or more heteroatoms independently selected from, e.g., N, O, and S. One or more heteroatoms may also be included in a substituent that replaced a hydrogen atom in a group or moiety as described herein. For example, in an optionally substituted heteroaryl group, if one of the hydrogen atoms in the heteroaryl group is replaced with a substituent (e.g., methyl), the substituent may also contain one or more heteroatoms (e.g., methanol).

The term “acyl,” as used herein, refers to a group having the structure:

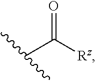

wherein R^{z} is an optionally substituted alkyl, alkenyl, alkynyl, cycloalkyl, cycloalkenyl, cycloalkynyl, aryl, alkaryl, alkamino, heteroalkyl, heteroalkenyl, heteroalkynyl, heterocycloalkyl, heterocycloalkenyl, heterocycloalkynyl, heteroaryl, heteroalkaryl, or heteroalkamino.

The term “halo” or “halogen,” as used herein, refers to any halogen atom, e.g., F, Cl, Br, or I. Any one of the groups or moieties described herein may be referred to as a “halo moiety” if it contains at least one halogen atom, such as haloalkyl.

The term “hydroxyl,” as used herein, represents an —OH group.

The term “oxo,” as used herein, refers to a substituent having the structure ═O, where there is a double bond between an atom and an oxygen atom.

The term “carbonyl,” as used herein, refers to a group having the structure:

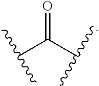 [SMILES: *C(*)=O]

The term “thiocarbonyl,” as used herein, refers to a group having the structure:

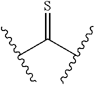 [SMILES: *C(*)=S]

The term “phosphate,” as used herein, represents the group having the structure:

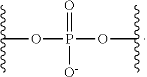 [SMILES: *OP(=O)([O-])O*]

The term “phosphoryl,” as used herein, represents the group having the structure:

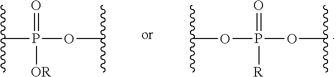

The term “sulfonyl,” as used herein, represents the group having the structure:

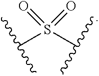 [SMILES: *S(*)(=O)=O]

The term “imino,” as used herein, represents the group having the structure:

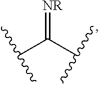

wherein R is an optional substituent.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 5th Edition (John Wiley & Sons, New York, 2014), which is incorporated herein by reference. N-protecting groups include, e.g., acyl, aryloyl, and carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, carboxybenzyl (CBz), 4-chlorobenzoyl, 4-bromobenzoyl, 4-nitrobenzoyl, and chiral auxiliaries such as protected or unprotected D, L or D, L-amino acid residues such as alanine, leucine, phenylalanine; sulfonyl-containing groups such as benzenesulfonyl and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl (BOC), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl (Fmoc), cyclopentyloxycarbonyl, adamantyloxycarbonyl, cyclohexyloxycarbonyl, and phenylthiocarbonyl; alkaryl groups such as benzyl, triphenylmethyl, and benzyloxymethyl; and silyl groups such as trimethylsilyl.

The term “amino acid,” as used herein, means naturally occurring amino acids and non-naturally occurring amino acids.

The term “naturally occurring amino acids,” as used herein, means amino acids including Ala, Arg, Asn, Asp, Cys, Gln, Glu, Gly, His, Ile, Leu, Lys, Met, Phe, Pro, Ser, Thr, Trp, Tyr, and Val.

The term “non-naturally occurring amino acid,” as used herein, means an alpha amino acid that is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxomethionine; phenylglycine; citrulline; methionine sulfoxide; cysteic acid; ornithine; diaminobutyric acid; 3-aminoalanine; 3-hydroxy-D-proline; 2,4-diaminobutyric acid; 2-aminopentanoic acid; 2-aminooctanoic acid, 2-carboxy piperazine; piperazine-2-carboxylic acid, 2-amino-4-phenylbutanoic acid; 3-(2-naphthyl)alanine, and hydroxyproline. Other amino acids are α-aminobutyric acid, α-amino-α-methylbutyrate, aminocyclopropane-carboxylate, aminoisobutyric acid, aminonorbornyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-ornithine, L-N-methylethylglycine, L-norleucine, α-methyl-aminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N—(N-(2,2-diphenylethyl) carbamylmethylglycine, 1-carboxy-1-(2,2-diphenyl-ethylamino) cyclopropane, 4-hydroxyproline, ornithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, O-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)₄-hydroxypiperidine-2-carboxylic acid, (2S,4S) and (2S,4R)-4-(2-naphthylmethoxy)-pyrolidine-2-carboxylic acid, (2S,4S) and (2S,4R)4-phenoxy-pyrrolidine-2-carboxylic acid, (2R,5S) and (2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisoleucine, L-N-methyllysine, L-N-methylnorleucine, L-N-methylornithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcyclopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cyclododecylglycine, N-cyclooctylglycine, N-cyclopropylglycine, N-cyclododecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorleucine, L-α-methylomithine, L-α-methylproline, L-α-methylthreonine, L-α-methyltyrosine, L-N-methylhomophenylalanine, N—(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenylalanine, L-3-pyridylalanine, L-histidine (benzoyloxymethyl), N-cycloheptylglycine, L-diphenylalanine, O-methyl-L-homotyrosine, L-β-homolysine, O-glycan-threonine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(benzothiazol-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R) and (2S,4S)-4-(4-phenylbenzyl) pyrrolidine-2-carboxylic acid, (3S)-1-pyrrolidine-3-carboxylic acid, (2S,4S)-4-tritylmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine. In some embodiments, amino acid residues may be charged or polar. Charged amino acids include alanine, lysine, aspartic acid, or glutamic acid, or non-naturally occurring analogs thereof. Polar amino acids include glutamine, asparagine, histidine, serine, threonine, tyrosine, methionine, or tryptophan, or non-naturally occurring analogs thereof. It is specifically contemplated that in some embodiments, a terminal amino group in the amino acid may be an amido group or a carbamate group.

As used herein, the term “percent (%) identity” refers to the percentage of amino acid residues of a candidate sequence, e.g., an Fc-IgG, or fragment thereof, that are identical to the amino acid residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid sequence identity of a given candidate sequence to, with, or against a given reference sequence (which can alternatively be phrased as a given candidate sequence that has or includes a certain percent amino acid sequence identity to, with, or against a given reference sequence) is calculated as follows:

100×(fraction of A/B)

where A is the number of amino acid residues scored as identical in the alignment of the candidate sequence and the reference sequence, and where B is the total number of amino acid residues in the reference sequence. In some embodiments where the length of the candidate sequence does not equal to the length of the reference sequence, the percent amino acid sequence identity of the candidate sequence to the reference sequence would not equal to the percent amino acid sequence identity of the reference sequence to the candidate sequence.

Two polynucleotide or polypeptide sequences are said to be “identical” if the sequence of nucleotides or amino acids in the two sequences is the same when aligned for maximum correspondence as described above. Comparisons between two sequences are typically performed by comparing the sequences over a comparison window to identify and compare local regions of sequence similarity. A “comparison window” as used herein, refers to a segment of at least about 15 contiguous positions, about 20 contiguous positions, about 25 contiguous positions, or more (e.g., about 30 to about 75 contiguous positions, or about 40 to about 50 contiguous positions), in which a sequence may be compared to a reference sequence of the same number of contiguous positions after the two sequences are optimally aligned.

The term “treating” or “to treat,” as used herein, refers to a therapeutic treatment of a viral infection (e.g., a viral infection such as an RSV infection) in a subject. In some embodiments, a therapeutic treatment may slow the progression of the viral infection, improve the subject's outcome, and/or eliminate the infection. In some embodiments, a therapeutic treatment of a viral infection in a subject may alleviate or ameliorate of one or more symptoms or conditions associated with the viral infection, diminish the extent of the viral, stabilize (i.e., not worsening) the state of the viral infection, prevent the spread of the viral infection, and/or delay or slow the progress of the viral infection, as compare the state and/or the condition of the viral infection in the absence of the therapeutic treatment.

The term “average value of T,” as used herein, refers to the mean number of monomers of RSV F protein inhibitor or dimers of RSV F protein inhibitors conjugated to an Fc domain or an albumin protein within a population of conjugates. In some embodiments, within a population of conjugates, the average number of monomers of RSV F protein inhibitor or dimers of RSV F protein inhibitors conjugated to an Fc domain monomer may be from 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20.

The term “subject,” as used herein, can be a human or non-human primate.

The term “therapeutically effective amount,” as used herein, refers to an amount, e.g., pharmaceutical dose, effective in inducing a desired effect in a subject or in treating a subject having a condition or disorder described herein (e.g., a viral infection, such as an RSV infection). It is also to be understood herein that a “therapeutically effective amount” may be interpreted as an amount giving a desired therapeutic and/or preventative effect, taken in one or more doses or in any dosage or route, and/or taken alone or in combination with other therapeutic agents (e.g., an antiviral agent described herein). For example, in the context of administering a conjugate described herein (e.g., a conjugate of any one of formulas (1), (2), (D-I)-(D-IV), or (M-I)-(M-IV)) that is used for the treatment of a viral infection, an effective amount of a conjugate is, for example, an amount sufficient to prevent, slow down, or reverse the progression of the viral infection as compared to the response obtained without administration of the conjugate.

As used herein, the term “pharmaceutical composition” refers to a medicinal or pharmaceutical formulation that contains at least one active ingredient (e.g., a conjugate of any one of formulas (1), (2), (D-I)-(D-IV), or (M-I)-(M-IV)) as well as one or more excipients and diluents to enable the active ingredient suitable for the method of administration. The pharmaceutical composition of the present disclosure includes pharmaceutically acceptable components that are compatible with a conjugate described herein (e.g., a conjugate of any one of formulas (1), (2), (D-I)-(D-IV), or (M-I)-(M-IV)).

As used herein, the term “pharmaceutically acceptable carrier” refers to an excipient or diluent in a pharmaceutical composition. For example, a pharmaceutically acceptable carrier may be a vehicle capable of suspending or dissolving the active conjugate (e.g., a conjugate of any one of formulas (1), (2), (D-I)-(D-IV), or (M-I)-(M-IV)). The pharmaceutically acceptable carrier must be compatible with the other ingredients of the formulation and not deleterious to the recipient. In the present disclosure, the pharmaceutically acceptable carrier must provide adequate pharmaceutical stability to a conjugate described herein. The nature of the carrier differs with the mode of administration. For example, for oral administration, a solid carrier is preferred; for intravenous administration, an aqueous solution carrier (e.g., WFI, and/or a buffered solution) is generally used.

The term “pharmaceutically acceptable salt,” as used herein, represents salts of the conjugates described herein (e.g., conjugates of any one of formulas (1), (2), (D-I)-(D-IV), or (M-I)-(M-IV)) that are, within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of the conjugates described herein or separately by reacting the free base group with a suitable organic acid.

The term “about,” as used herein, indicates a deviation of ±5%. For example, about 10% refers to from 9.5% to 10.5%.

Any values provided in a range of values include both the upper and lower bounds, and any values contained within the upper and lower bounds.

Other features and advantages of the conjugates described herein will be apparent from the following Detailed Description and the claims.

The term “(1), (2), (D-I)-(D-IV), or (M-I)-(M-IV)”, as used herein, represents the formulas of any one of (1), (2), (D-I)-(D-IV), or (M-I)-(M-IV) (e.g., any one of formulas (1), (2), (D-I), (D-II), (D-II-1), (D-II-2), (D-II-3), (D-II-4), (D-II-5), (D-II-6), (D-II-7), (D-II-8), (D-II-9), (D-II-10), (D-II-11), (D-II-12), (D-II-13), (D-II-14), (D-II-15), (D-II-16), (D-II-17), (D-III), (D-III-1), (D-III-2), (D-III-3), (D-IV), (D-IV-1), (D-IV-2), (D-IV-3), (D-IV-4), (D-IV-5), (D-IV-6), (D-IV-7), (D-IV-8), (D-IV-9), (D-IV-10), (D-IV-11), (D-IV-12), (D-IV-13), (D-IV-14), (D-IV-15), (D-IV-16), (D-IV-17), (D-IV-18), (M-I), (M-II), (M-II-17), (M-III), (M-III-1), (M-III-2), (M-III-3), (M-IV), (M-IV-1), (M-IV-2), (M-IV-3), (M-IV-4), (M-IV-5), (M-IV-6), (M-IV-7), (M-IV-8), (M-IV-9), (M-IV-10), (M-IV-11), (M-IV-12), (M-IV-13), (M-IV-14), (M-IV-15), (M-IV-16), (M-IV-17), (M-IV-18).

### DESCRIPTION OF THE DRAWINGS

FIG. 1 is an image depicting exemplary methods of conjugating a RSV F protein inhibitor monomer or dimer, e.g., by way of a linker, to an Fc domain monomer, an Fc domain, an Fc-binding peptide, an albumin protein, or an albumin protein-binding peptide.

FIG. 2 is an image depicting a method of conjugating a RSV F protein inhibitor monomer or dimer, e.g., by way of a linker, to an Fc domain monomer, an Fc domain, an Fc-binding peptide, an albumin protein, or an albumin protein-binding peptide by oxime conjugation to an amino acid residue, e.g., a nitrogen atom of a surface exposed lysine.

FIG. 3 is an image depicting a method of conjugating a RSV F protein inhibitor monomer or dimer, e.g., by way of a linker, to an Fc domain monomer, an Fc domain, an Fc-binding peptide, an albumin protein, or an albumin protein-binding peptide by thioether conjugation to an amino acid residue, e.g., a nitrogen atom of a surface exposed lysine.

FIG. 4 is an image depicting a method of conjugating a RSV F protein inhibitor monomer or dimer, e.g., by way of a linker, to an Fc domain monomer, an Fc domain, an Fc-binding peptide, an albumin protein, or an albumin protein-binding peptide by rebridged cysteine conjugation, e.g., rebridged cysteine conjugation to a pair of sulfur atoms of two hinge cysteines in an Fc domain monomer or Fc domain.

FIG. 5 shows non-reducing and reducing SDS-PAGE and a schematic illustration of an Fc domain formed from Fc domain monomers having the sequence of SEQ ID NO: 1.

FIG. 6 shows non-reducing and reducing SDS-PAGE and a schematic illustration of an Fc domain formed from Fc domain monomers having the sequence of SEQ ID NO: 3.

FIG. 7 shows non-reducing and reducing SDS-PAGE and a schematic illustration of an Fc domain formed from Fc domain monomers having the sequence of SEQ ID NO: 5.

FIG. 8 shows non-reducing and reducing SDS-PAGE and a schematic illustration of an Fc domain formed from Fc domain monomers having the sequence of SEQ ID NO: 7.

FIG. 9 shows non-reducing and reducing SDS-PAGE and a schematic illustration of an Fc domain formed from Fc domain monomers having the sequence of SEQ ID NO: 9.

FIG. 10 shows non-reducing and reducing SDS-PAGE and a schematic illustration of an Fc domain formed from Fc domain monomers having the sequence of SEQ ID NO: 12.

FIG. 11 shows non-reducing and reducing SDS-PAGE and a schematic illustration of an Fc domain formed from Fc domain monomers having the sequence of SEQ ID NO: 14.

FIG. 12 is a graph showing the binding of conjugate 6 to the RSV F protein compared to an unconjugated Fc molecule negative control.

FIG. 13 is a graph showing plasma levels of a conjugate including an Fc domain having a C220S mutation (SEQ ID NO: 64) (2 mpk IV) compared to a conjugate including an Fc domain having a C220S mutation and a YTE triple mutation (SEQ ID NO: 67) (2 mpk IV) in non-human primate PK studies determined by Fc capture. This study was performed as described in Example 56.

FIG. 14 is a graph showing plasma concentration levels of a conjugate including an Fc domain having a C220S mutation (SEQ ID NO: 64) compared to epithelial lining fluid (ELF) levels of the conjugate in mice. This study was performed as described in Example 57.

FIG. 15 is an image depicting exemplary conjugates of an RSV F protein inhibitor monomer or dimer and an Fc domain monomer or an Fc domain. “T” is representative of the drug-to-antibody ratio (DAR) and depicts that multiple monomers or dimers can be conjugated to each Fc domain monomer or Fc domain.

FIG. 16 is an image depicting exemplary conjugates of an RSV F protein inhibitor monomer or dimer and an Fc domain monomer or an Fc domain. “T” is representative of the drug-to-antibody ratio (DAR) and depicts that multiple monomers or dimers can be conjugated to each Fc domain monomer or Fc domain.

### DETAILED DESCRIPTION

The disclosure features conjugates, compositions, and methods for the treatment of viral infections (e.g., RSV such as RSV A or RSV B). The conjugates disclosed herein include monomers or dimers of viral RSV F protein inhibitors (e.g., Presatovir, MDT 637, JNJ 179, or an analog thereof) conjugated to Fc monomers, Fc domains, Fc-binding peptides, albumin proteins, or albumin protein-binding peptides. The RSV F protein inhibitor (e.g., Presatovir, MDT 637, JNJ 179, or an analog thereof) in the conjugates targets RSV F protein on the surface of the viral particle. The Fc monomers or Fc domains in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC), thus leading to the engulfment and destruction of viral particles by immune cells and further enhancing the antiviral activity of the conjugates. The albumin or albumin-binding peptide may extend the half-life of the conjugate, for example, by binding of albumin to the recycling neonatal Fc receptor. Such compositions are useful in methods for the inhibition of viral growth and in methods for the treatment of viral infections, such as those caused by an RSV A and RSV B.

The featured conjugates exhibit desirable tissue distribution (e.g., lung distribution). Such compositions are therefore useful in methods for the treatment of disorders (e.g., respiratory disorders, inhibition of infection growth, and in methods for the treatment of infections (e.g., viral infections (e.g., RSV such as RSV A or RSV B).

### I. Viral Infections

The compounds and pharmaceutical compositions described herein (e.g., a conjugate of any one of formulas (1), (2), (D-I)-(D-IV), or (M-I)-(M-IV)) can be used to treat a viral infection (e.g., an RSV A or RSV B viral infection).

Viral infection refers to the pathogenic growth of a virus (e.g., RSV such as RSV A or RSV B) in a host organism (e.g., a human subject). A viral infection can be any situation in which the presence of a viral population(s) is damaging to a host body. Thus, a subject is suffering from a viral infection when an excessive amount of a viral population is present in or on the subject's body, or when the presence of a viral population(s) is damaging the cells or other tissue of the subject.

Human respiratory syncytial virus (RSV) is a medium-sized (120-200 nm) enveloped virus that contains a lipoprotein coat and a linear negative-sense RNA genome (must be converted to a positive RNA prior to translation). The former contains virally encoded F, G, and SH lipoproteins. The F and G lipoproteins are the only two that target the cell membrane, and are highly conserved among RSV isolates. Human RSV (HRSV) is divided into two antigenic subgroups, A and B, on the basis of the reactivity of the virus with monoclonal antibodies against the attachment (G) and fusion (F) glycoproteins. Subtype B is characterized as the asymptomatic strains of the virus that the majority of the population experiences. The more severe clinical illnesses involve subtype A strains, which tend to predominate in most outbreaks.

Four of the viral genes code for intracellular proteins that are involved in genome transcription, replication, and particle budding, namely N (nucleoprotein), P (phosphoprotein), M (matrix protein), and L (“large” protein, containing the RNA polymerase catalytic motifs). The RSV genomic RNA forms a helical ribonucleoprotein (RNP) complex with the N protein, termed nucleocapsid, which is used as template for RNA synthesis by the viral polymerase complex. The three-dimensional crystal structure of a decameric, annular ribonucleoprotein complex of the RSV nucleoprotein (N) bound to RNA has been determined at 3.3 Å resolution. This complex mimics one turn of the viral helical nucleocapsid complex. Its crystal structure was combined with electron microscopy data to provide a detailed model for the RSV nucleocapsid.

### II. Conjugates of the Disclosure

Provided herein are synthetic conjugates useful in the treatment of viral infections (e.g., RSV infections). The conjugates disclosed herein include an Fc domain or an albumin protein conjugated to one or more monomers RSV F protein inhibitors or one or more dimers of two RSV F protein inhibitors (e.g., RSV F protein inhibitors selected from Presatovir, MDT 637, JNJ 179, or an analog thereof). The dimers of two RSV F protein inhibitors include a RSV F protein inhibitor (e.g., a first RSV F protein inhibitor of formula (A-I), (A-II), or (A-III)) and a second RSV F protein inhibitor (e.g., a second RSV F protein inhibitor of formula (A-I), (A-II), or (A-III)). The first and second RSV F protein inhibitors are linked to each other by way of a linker.

Without being bound by theory, in some aspects, conjugates described herein bind to the surface of a viral particle (e.g., bind to viral RSV F protein on the surface an RSV particle) through the interactions between the RSV F protein inhibitor moieties in the conjugates and proteins on the surface of the viral particle. The RSV F protein inhibitor disrupts RSV F protein-mediated fusion with the host cell membrane, preventing viral entry. The process of membrane fusion begins once prefusion RSV F protein is triggered by an unknown mechanism to initiate a dramatic conformational change, refolding and bringing the RSV and host cell membrane together.

Conjugates of the invention include RSV F protein inhibitor monomers and dimers conjugated to an Fc domain, Fc monomer, or Fc-binding peptide. The Fc domain in the conjugates described herein binds to the FcγRs (e.g., FcRn, FcγRI, FcγRIIa, FcγRIIc, FcγRIIIa, and FcγRIIIb) on immune cells. The binding of the Fc domain in the conjugates described herein to the FcγRs on immune cells activates phagocytosis and effector functions, such as antibody-dependent cell-mediated cytotoxicity (ADCC), thus leading to the engulfment and destruction of viral particles by immune cells and further enhancing the antiviral activity of the conjugates.

Conjugates of the invention include RSV F protein inhibitor monomers and dimers conjugated to an albumin protein or an albumin protein-binding peptide. The albumin protein or albumin protein-binding peptide may extend the half-life of the conjugate, for example, by binding of albumin to the recycling neonatal Fc receptor.

Conjugates provided herein are described by any one of formulas (1), (2), (D-I)-(D-IV), or (M-I)-(M-IV). In some embodiments, the conjugates described herein include one or more monomers of RSV F protein inhibitors conjugated to an Fc domain or an albumin protein. In some embodiments, the conjugates described herein include one or more dimers of RSV F protein inhibitors conjugated to an Fc domain or an albumin protein. In some embodiments, when n is 2, E (an Fc domain monomer) dimerizes to form an Fc domain.

Conjugates described herein may be synthesized using available chemical synthesis techniques in the art. In cases where a functional group is not available for conjugation, a molecule may be derivatized using conventional chemical synthesis techniques that are well known in the art. In some embodiments, the conjugates described herein contain one or more chiral centers. The conjugates include each of the isolated stereoisomeric forms as well as mixtures of stereoisomers in varying degrees of chiral purity, including racemic mixtures. It also encompasses the various diastereomers, enantiomers, and tautomers that can be formed.

### RSV F Protein Inhibitors

A component of the conjugates described herein is an RSV F protein inhibitor moiety. The RSV F protein inhibitor disrupts RSV F protein, an envelope glycoprotein that causes the virion membrane to fuse with a target cell membrane. The functional F protein trimer in the virion membrane is in a metastable, prefusion form. It is not yet clear what causes the F protein to trigger, but the result is a major refolding into its post fusion form. At the N-terminus of each F1 subunit is the fusion peptide (FP), a stretch of hydrophobic residues that insert into the target membrane. The FP is mirrored by the transmembrane (TM) domain near the C-terminus of F1, and each is connected to a heptad repeat (HR) in this order: FP-HRA-HRB-TM. Upon triggering the pre-HRA refolds into the long HRA helix and trimerizes. The RSV F protein folds in the center as the target and viral membranes approach each other, enabling HRB to bind to the grooves in the HRA trimer, forming a hairpin 6-helix bundle (6HB). Examples of RSV F protein inhibitors include Presatovir, MDT 637, JNJ 179. In addition, derivatives of Presatovir, MDT 637, JNJ 179, such as those found in the literature, have RSV F protein inhibitor activity and are useful as RSV F protein inhibitor moieties of the compounds herein (see, for example, Cockerill et al. J. Med. Chem. 62(7): 3206-3227, 2018).

Conjugates described herein are separated into two types: (1) one or more dimers of RSV F protein inhibitors conjugated to an Fc domain or an albumin protein and (2) one or more monomers of RSV F protein inhibitors conjugated to an Fc domain or an albumin protein. The dimers of RSV F protein inhibitors are linked to each other by way of a linker, such as the linkers described herein.

Viral RSV F protein inhibitors of the invention include Presatovir, MDT 637, JNJ 179, and analogs thereof, such as the viral RSV F protein inhibitors of formulas (A-I)-(A-III):

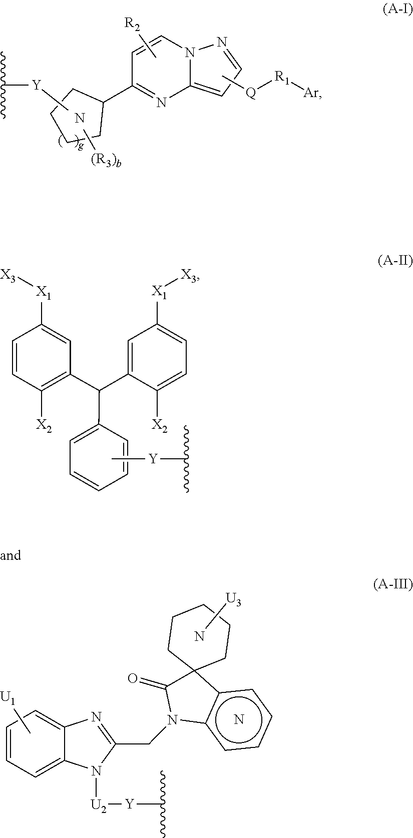

wherein

Q is selected from optionally substituted C₁-C₂₀ alkyl, optionally substituted C₃-C₂₀ cycloalkyl, optionally substituted C₂-C₂₀ heterocycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;

R₁, each X₁, and Y are each independently selected from —O—, —S—, —NR₅—, —CH═N—, —C(C═O)O—, —(C═O)NH—, —(C═O)—, —O(C═O)NR₅—, —O(C═S)NR₅—, —O(C═O)O—, —O(C═O)—, —NH(C═O)O—, —NH(C═O)—, —NH(C═NH)—, —NH(C═O)NR₅—, —NH(C═NH)NR₅—, —NH(C═S)NR₅—, —NH(C═S)—, —OCH₂(C═O)NR₅—, —R₅OR₆C(═O)NH—, —R₅NH(C═O)—, —R₅N—, —NH(SO₂)—, —NH(SO₂)NR₅—, —OR₆—, —NHR₆—, —SO₂— and —SR₆—;

R₂, each R₃, each X₂, and U₁, are each independently selected from OH, halogen, nitrile, nitro, optionally substituted amine, optionally substituted imine, optionally substituted C₁-C₂₀ alkamino, optionally substituted sulfhydryl, optionally substituted carboxyl, optionally substituted cyano, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;

each X₃ is independently selected from optionally substituted C₁-C₂₀ alkyl, optionally substituted C₃-C₂₀ cycloalkyl, optionally substituted C₂-C₂₀ heterocycloalkyl; optionally substituted C₅-C₁₅ aryl, and optionally substituted C₁-C₁₅ heteroaryl;

U₂ is a substituent of the ring nitrogen atom and is selected from optionally substituted C₁-C₂₀ alkyl, optionally substituted C₃-C₂₀ cycloalkyl, optionally substituted C₂-C₂₀ heterocycloalkyl, optionally substituted C₅-C₁₅ aryl, optionally substituted C₃-C₁₅ heteroaryl, and a bond;

U₃ is a substituent of ring nitrogen atom and is selected from H, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy, optionally substituted C₁-C₂₀ alkamino, optionally substituted carboxyl, optionally substituted cyano;

Ar is selected from optionally substituted C₃-C₂₀ cycloalkyl, optionally substituted C₂-C₂₀ heterocycloalkyl, optionally substituted C₅-C₁₅ aryl, and optionally substituted C₁-C₁₅ heteroaryl;

R₅ and R₆ are each independently selected from H, optionally substituted C₁-C₂₀ alkyl, optionally substituted C₃-C₂₀ cycloalkyl, optionally substituted C₂-C₂₀ heterocycloalkyl; optionally substituted C₅-C₁₅ aryl, and optionally substituted C₂-C₁₅ heteroaryl.

Preferably the RSV F protein inhibitor is selected from Presatovir (described, for example, as Compound 202 in U.S. Pat. No. 8,486,938), MDT 637 (described, for example, in Example 13 of U.S. Pat. No. 6,495,580), JNJ 179 (described, for example, as Compound 179 of International Patent Publication No. WO 2014/060411):

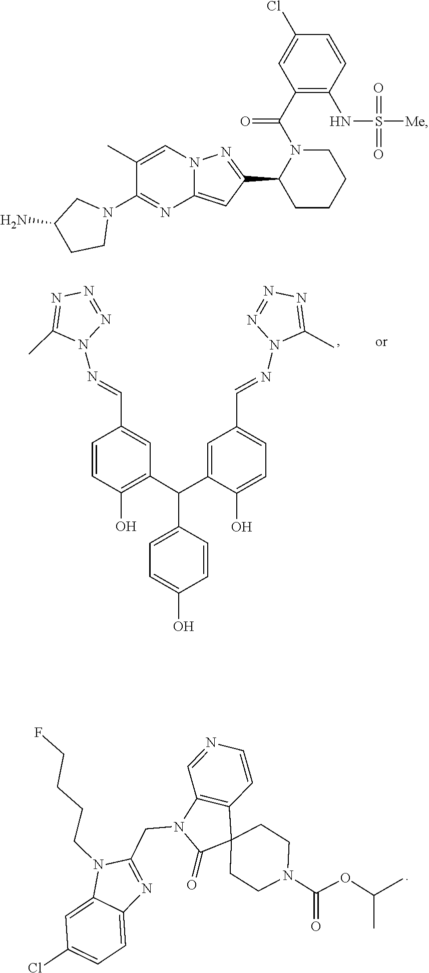

### Conjugates of Dimers of RSV F Protein Inhibitors Linked to an Fc Domain or an Albumin Protein

The conjugates described herein include an Fc domain, and Fc monomer, an Fc-binding peptide, and albumin protein, or an albumin protein-binding peptide covalently linked to one or more dimers of RSV F protein inhibitors. The dimers of two RSV F protein inhibitors include a first RSV F protein inhibitor (e.g., a first viral RSV F protein inhibitor of formulas (A-I)-(A-III)) and a second RSV F protein inhibitor (e.g., a second viral RSV F protein inhibitor of formulas (A-I)-(A-III)). The first and second RSV F protein inhibitors are linked to each other by way of a linker, such as a linker described herein. In some embodiments of the dimers of RSV F protein inhibitors, the first and second RSV F protein inhibitors are the same. In some embodiments, the first and second RSV F protein inhibitors are different.

In some embodiments, when T is greater than 1 (e.g., T is 2, 3, 4, 5, 6, 7, 8, 9, 10, 11, 12, 13, 14, 15, 16, 17, 18, 19, or 20), each A₁-L-A₂ may be independently selected (e.g., independently selected from any of the A₁-L-A₂ structures described herein). In some embodiments, E may be conjugated to 2, 3, 4, 5, 6, 7, 8, 9, 10, or more different A₁-L-A₂ moieties. In some embodiments, E is conjugated to a first A₁-L-A₂ moiety, and a second A₁-L-A₂, moiety. In some embodiments, each of A₁ and A₂ of the first A₁-L-A₂ moiety and of the second A₁-L-A₂ moiety are independently selected from any one of formulas (A-I)-(A-III):

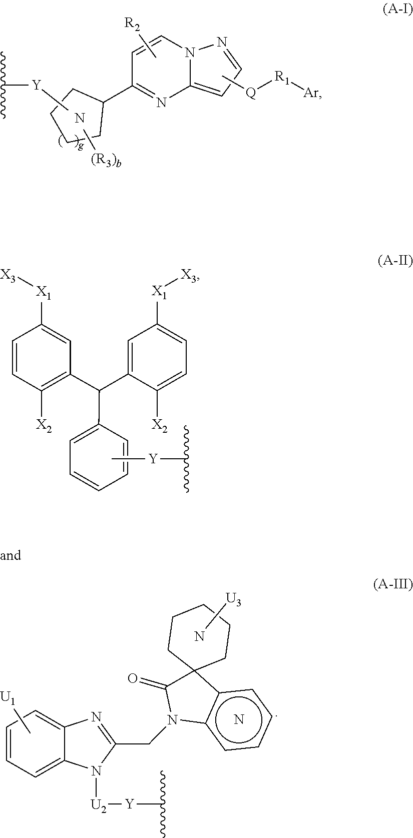

In some embodiments, the first A₁-L-A₂ moiety is conjugated specifically to lysine residues of E (e.g., the nitrogen atoms of surface exposed lysine residues of E), and the second A₁-L-A₂ moiety is conjugated specifically to cysteine residues of E (e.g., the sulfur atoms of surface exposed cysteine residues of E). In some embodiments, the first A₁-L-A₂ moiety is conjugated specifically to cysteine residues of E (e.g., the sulfur atoms of surface exposed cysteine residues of E), and the second A₁-L-A₂ moiety is conjugated specifically to lysine residues of E (e.g., the nitrogen atoms of surface exposed lysine residues of E).

In some embodiments, the disclosure provides a conjugate, or a pharmaceutically acceptable salt thereof, described by the formulae below:

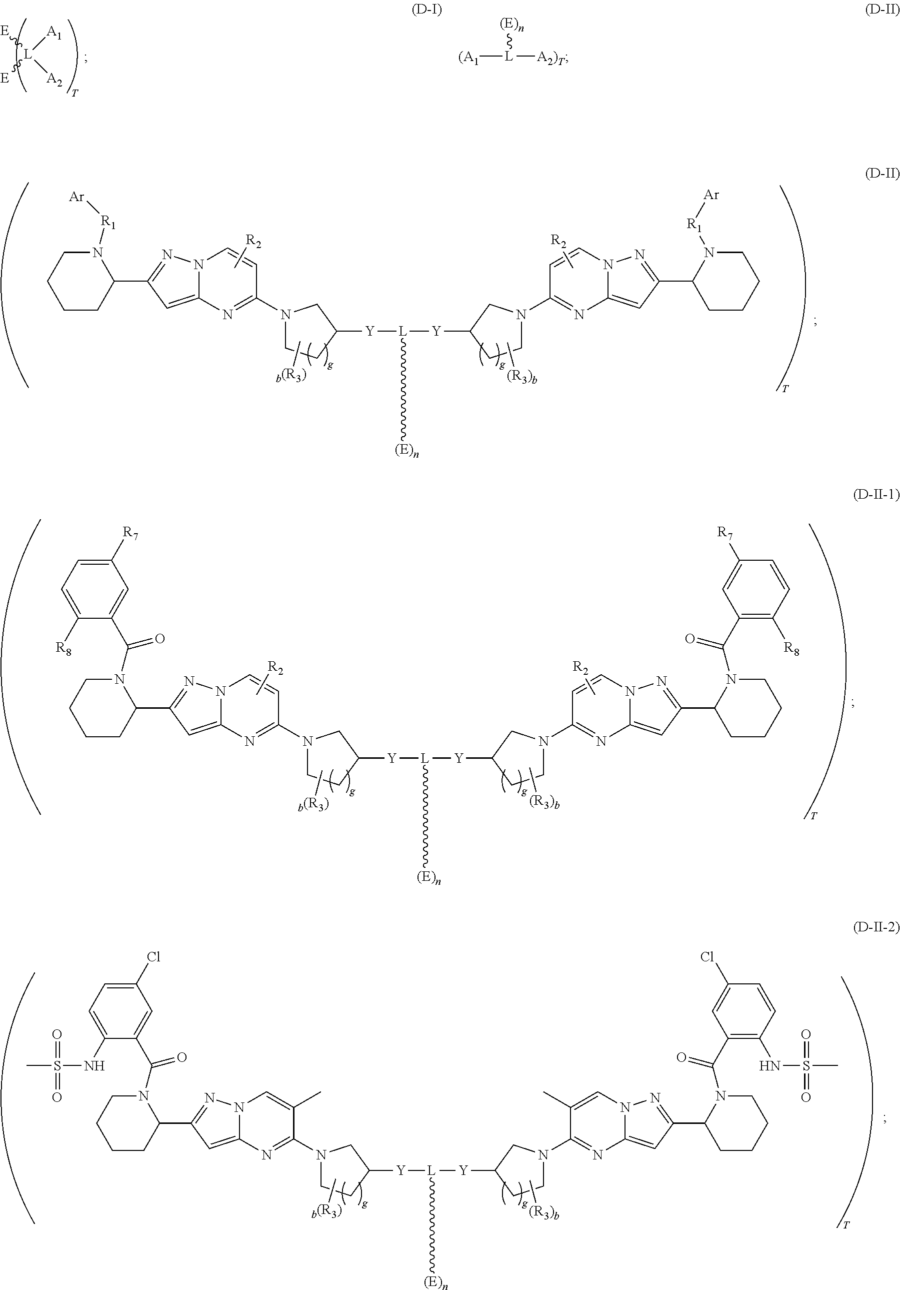

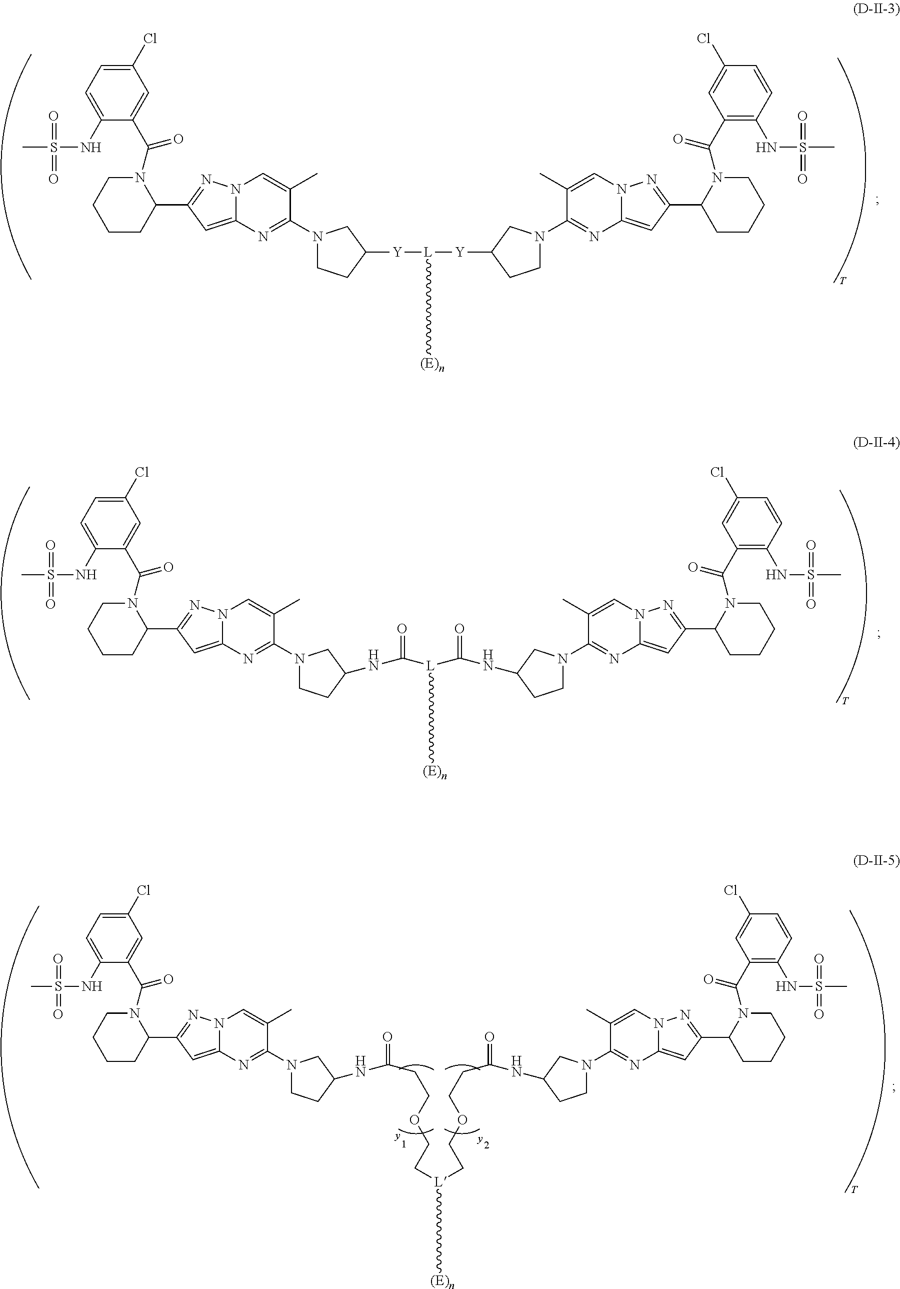

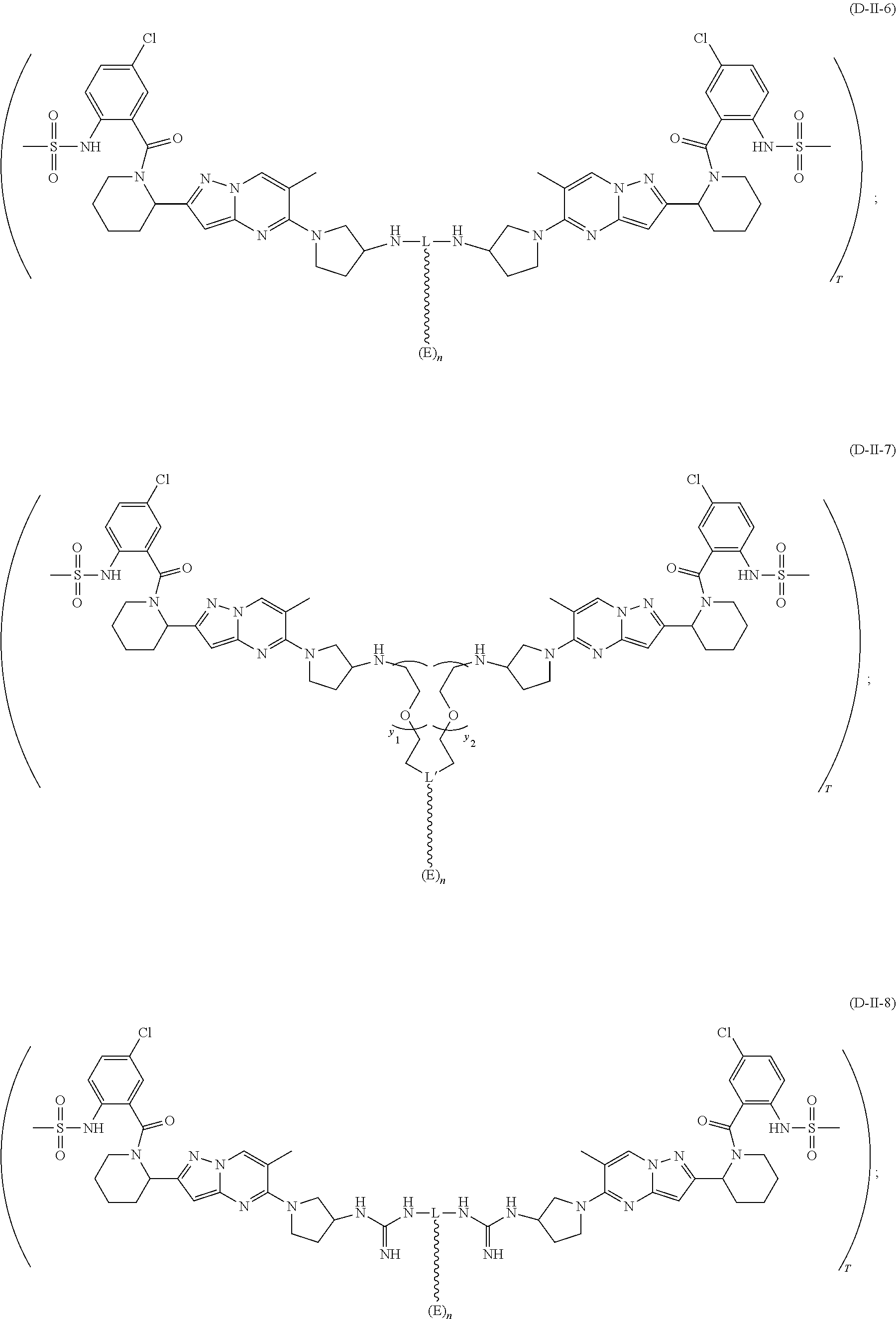

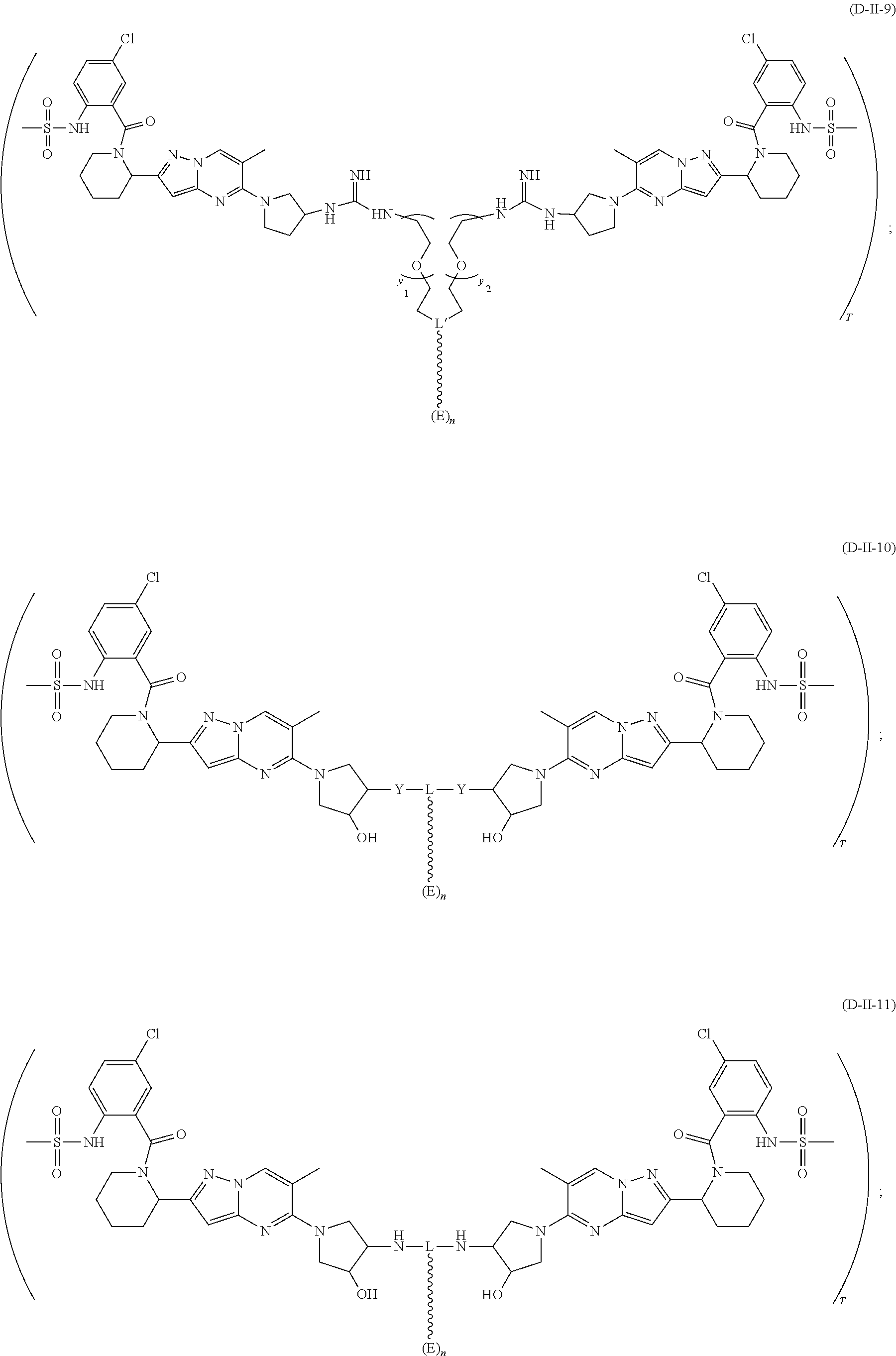

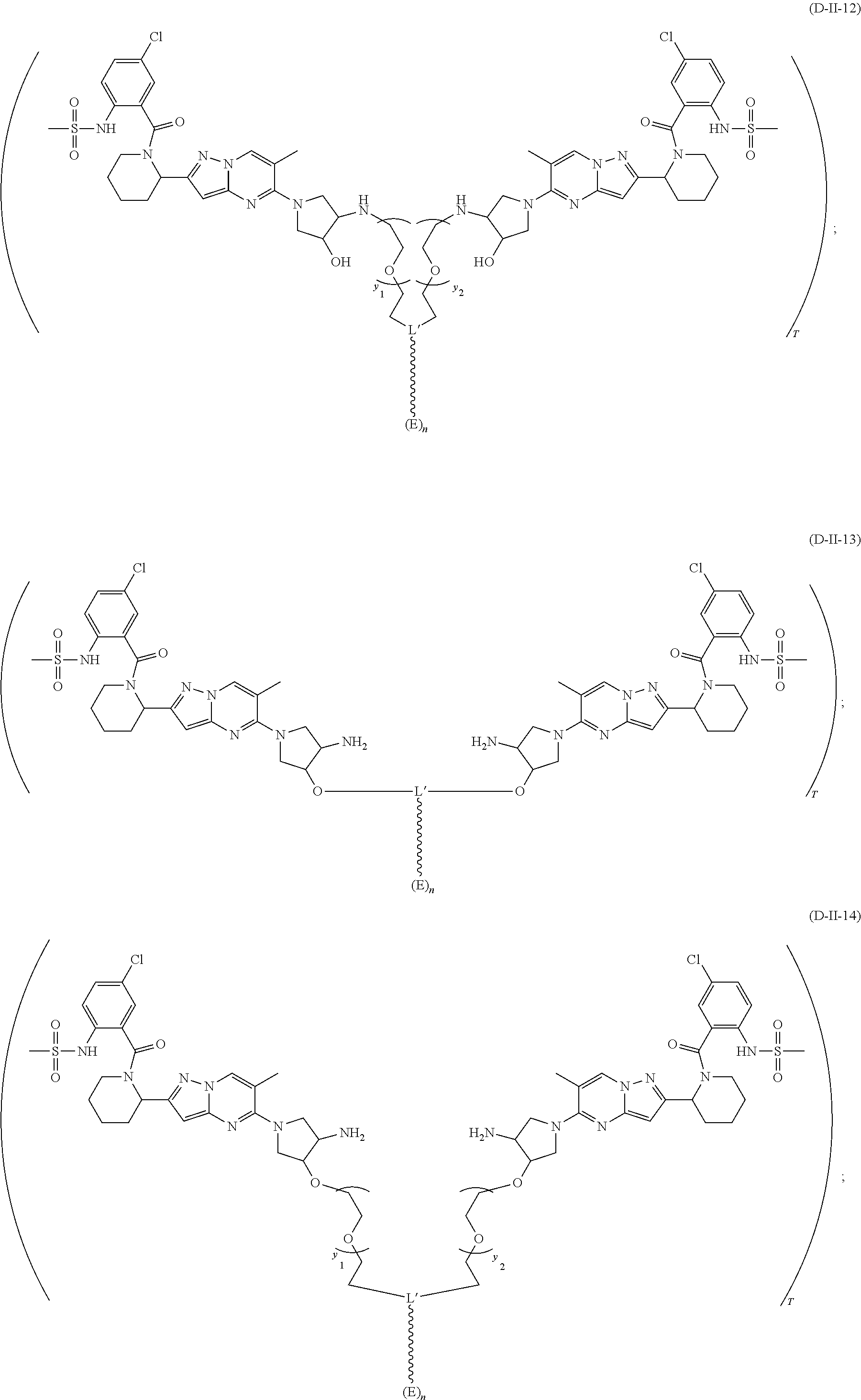

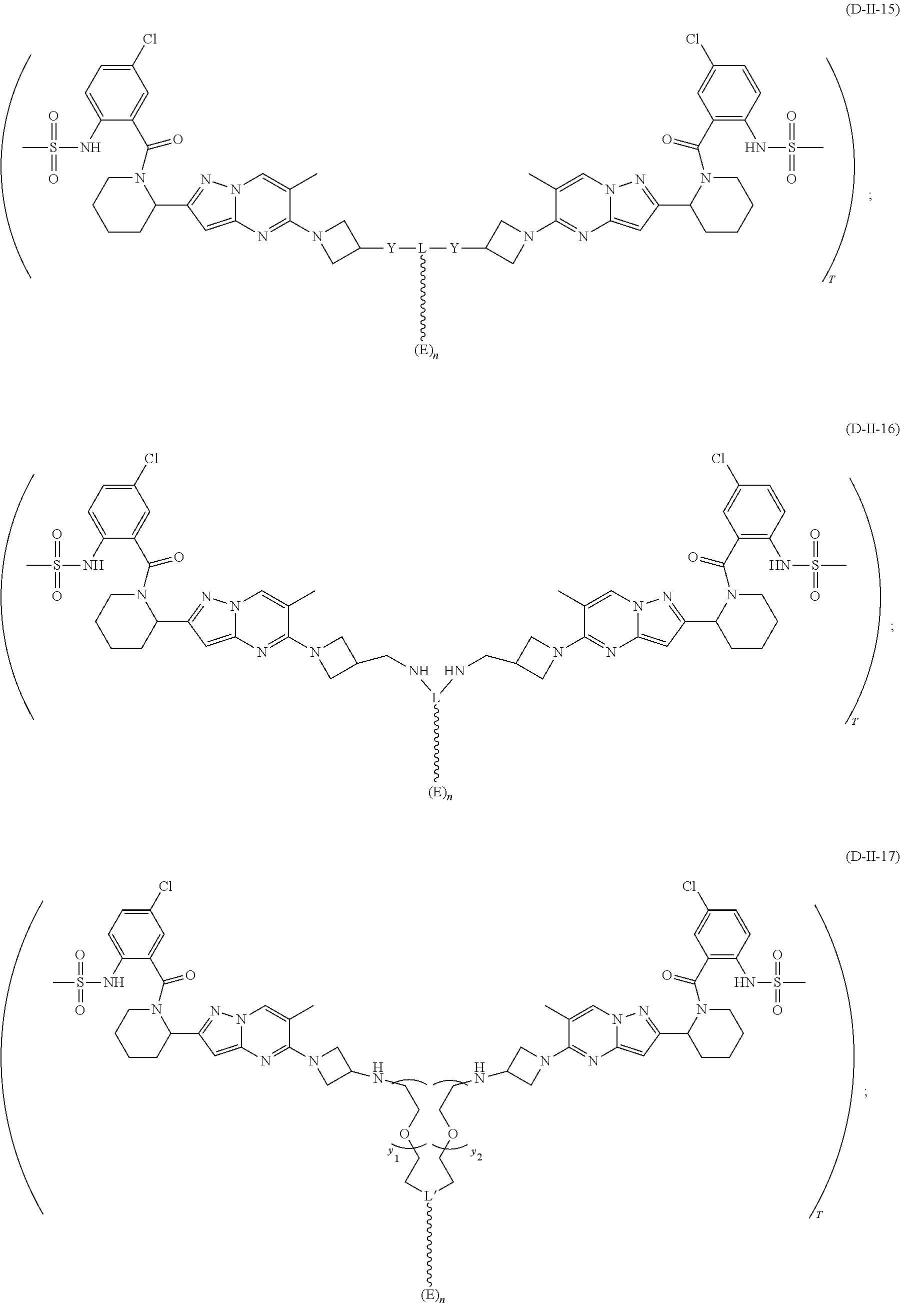

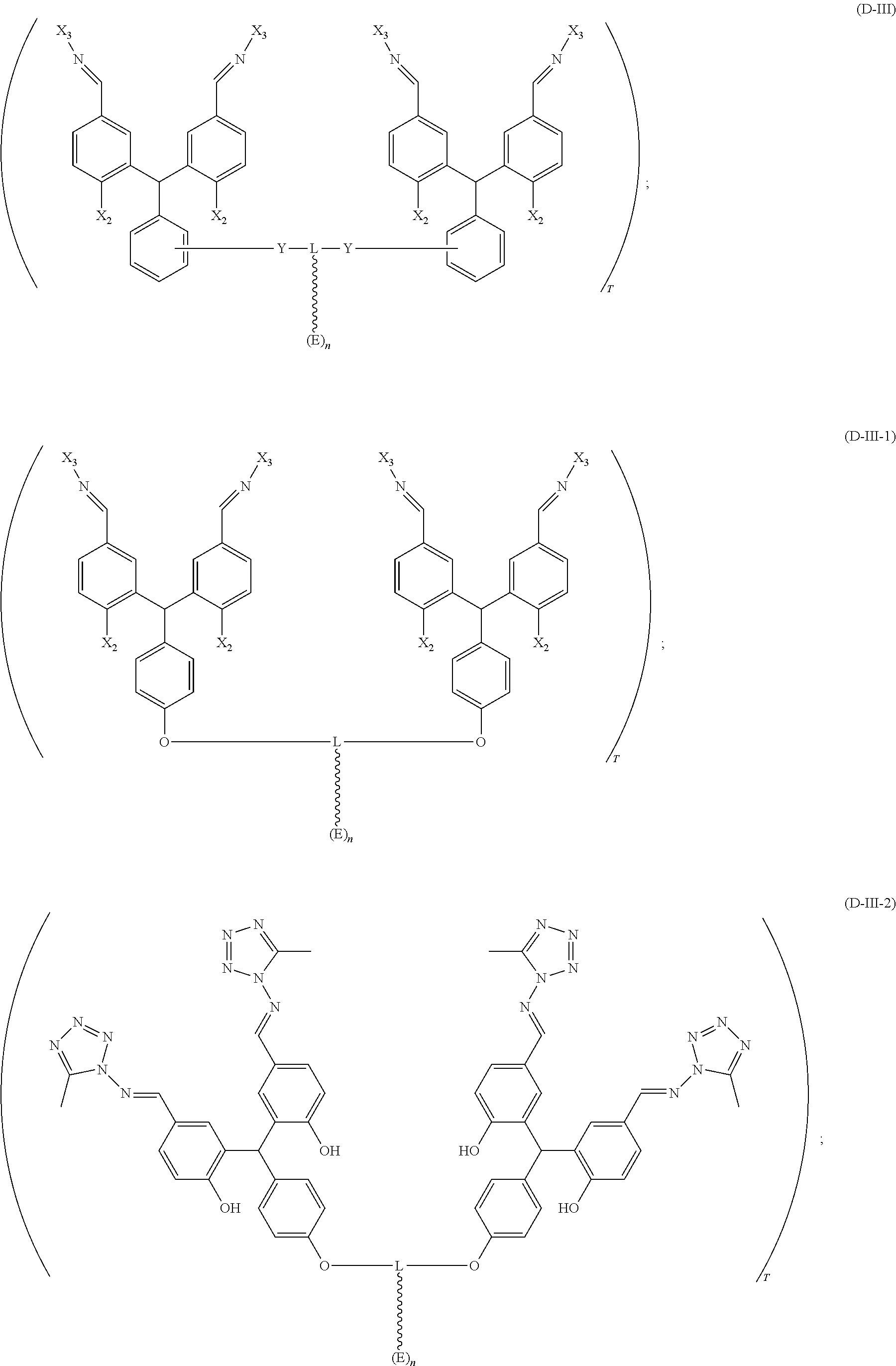

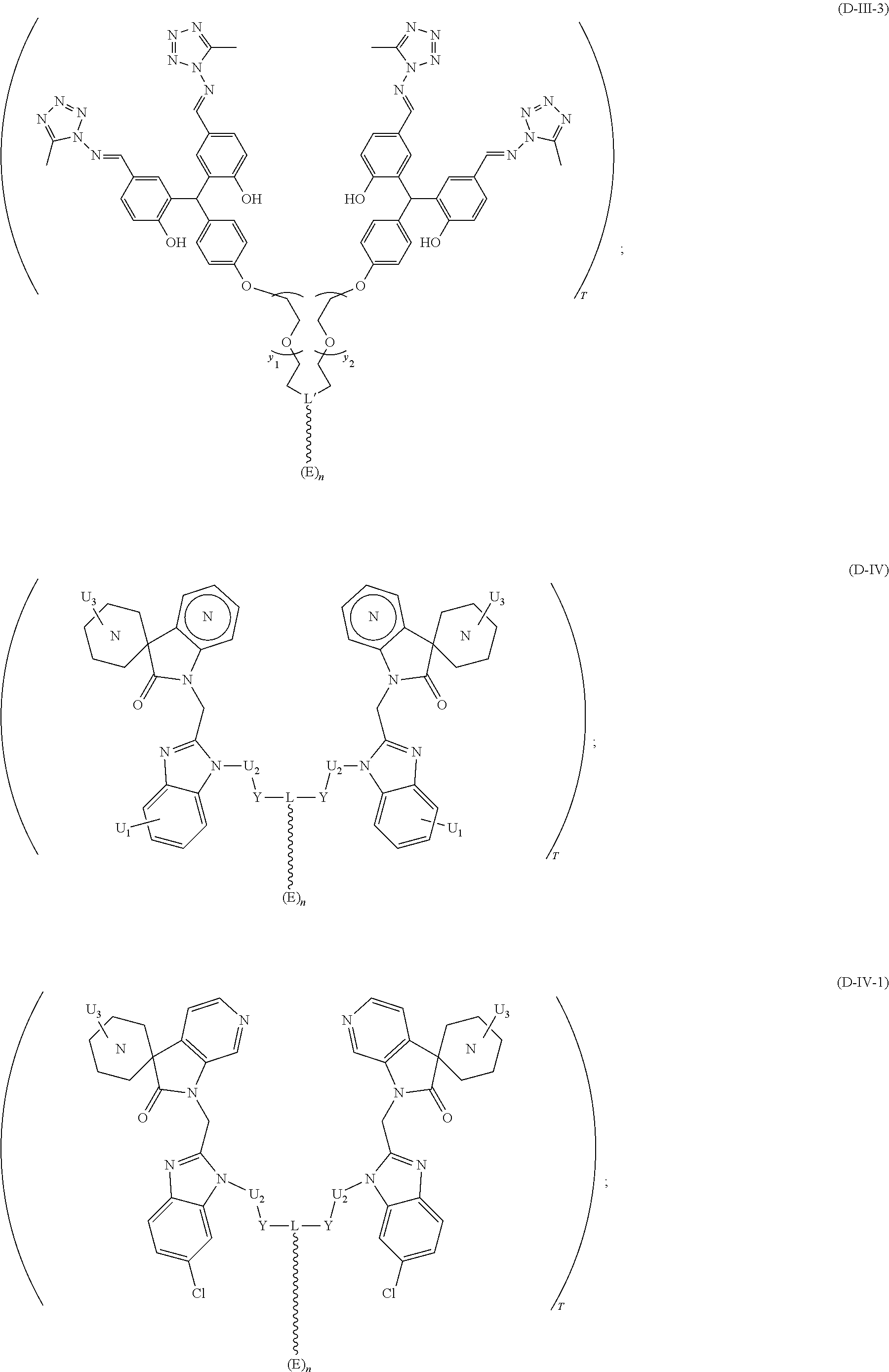

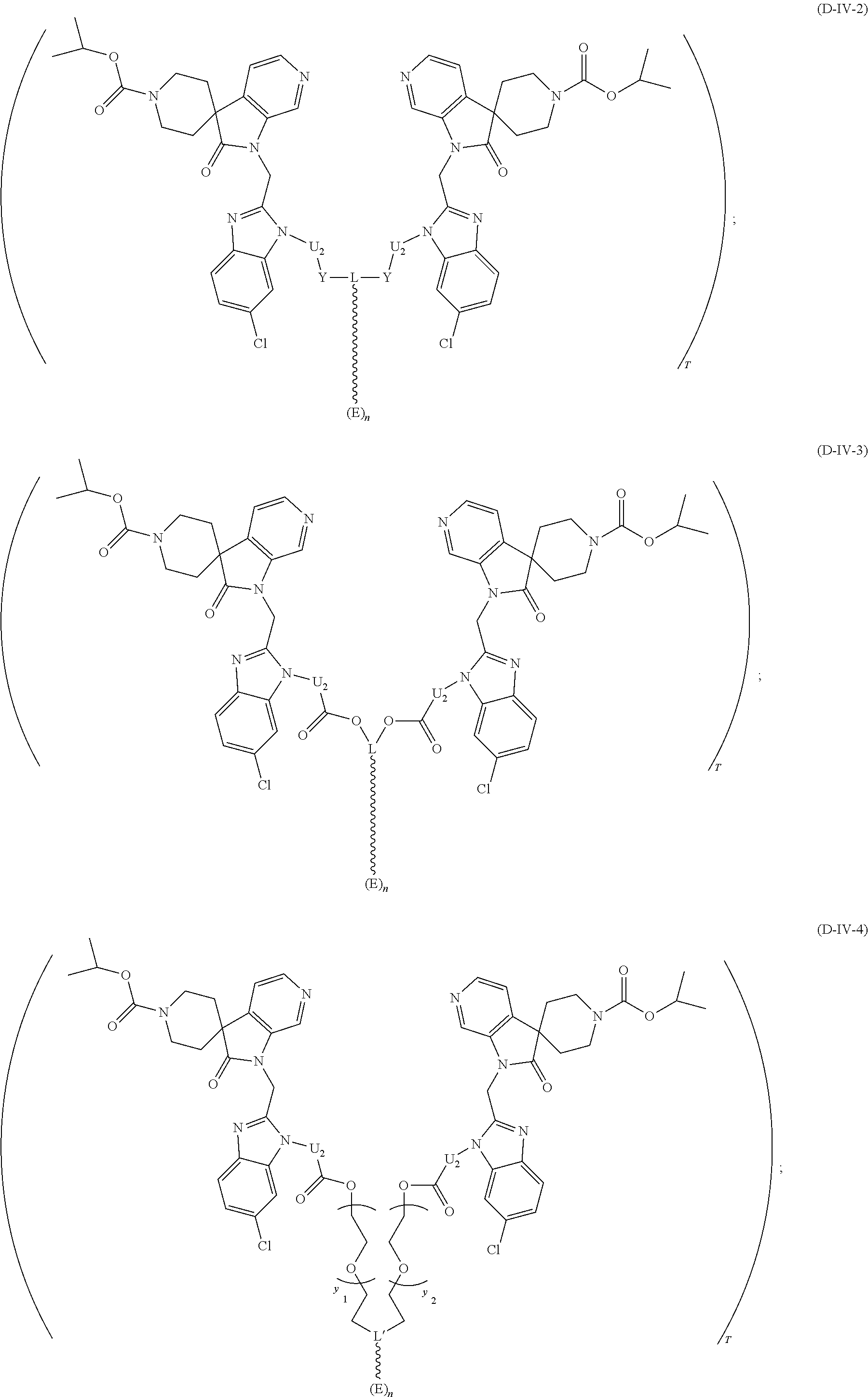

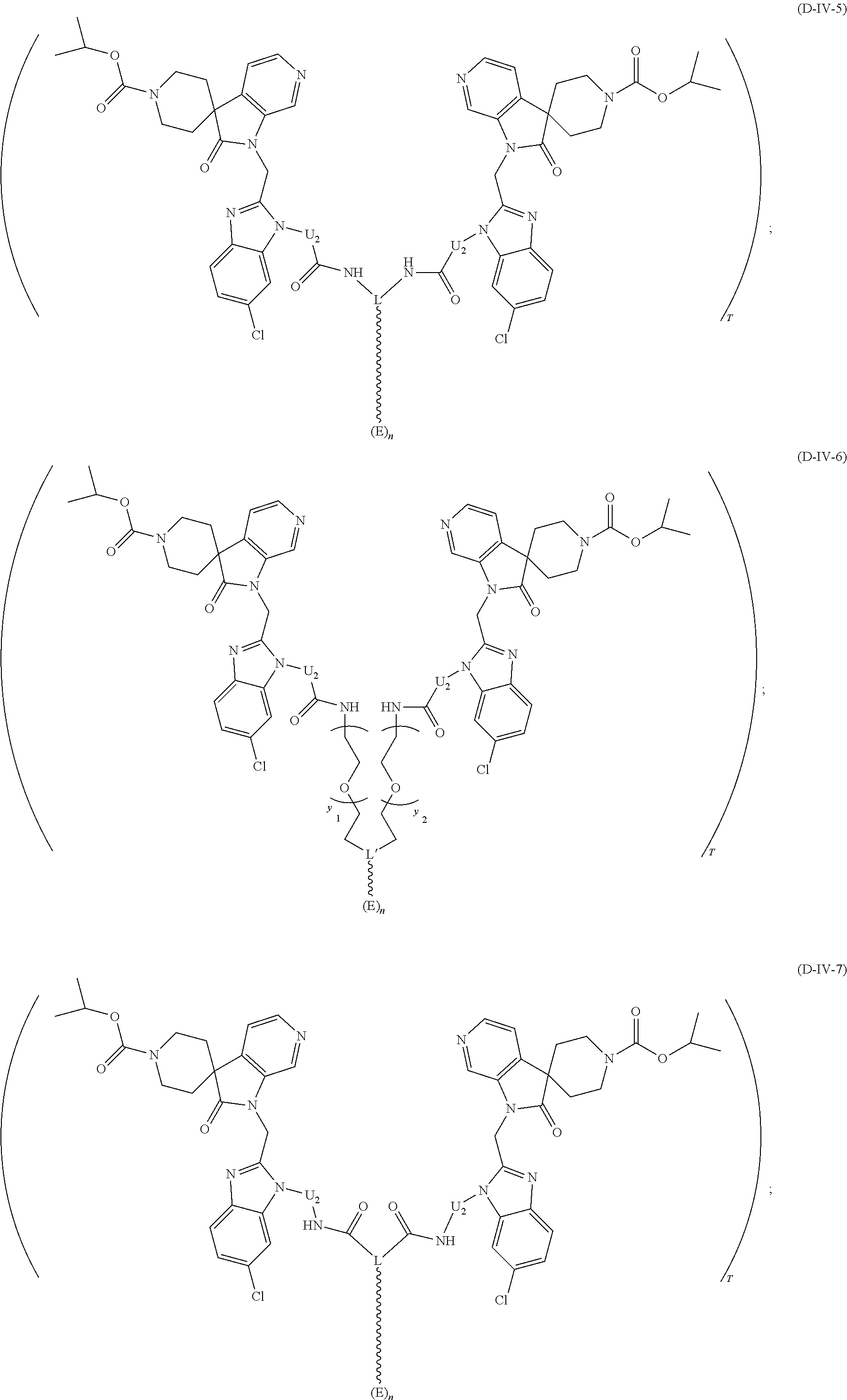

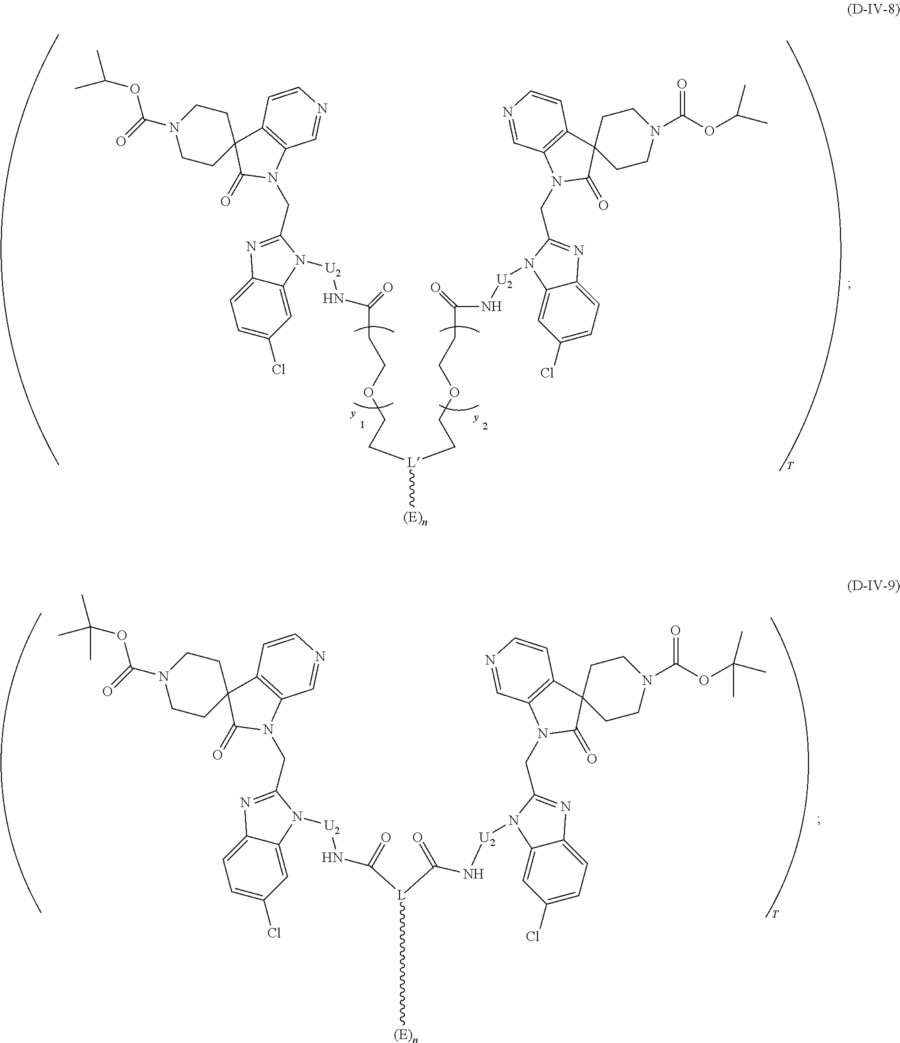

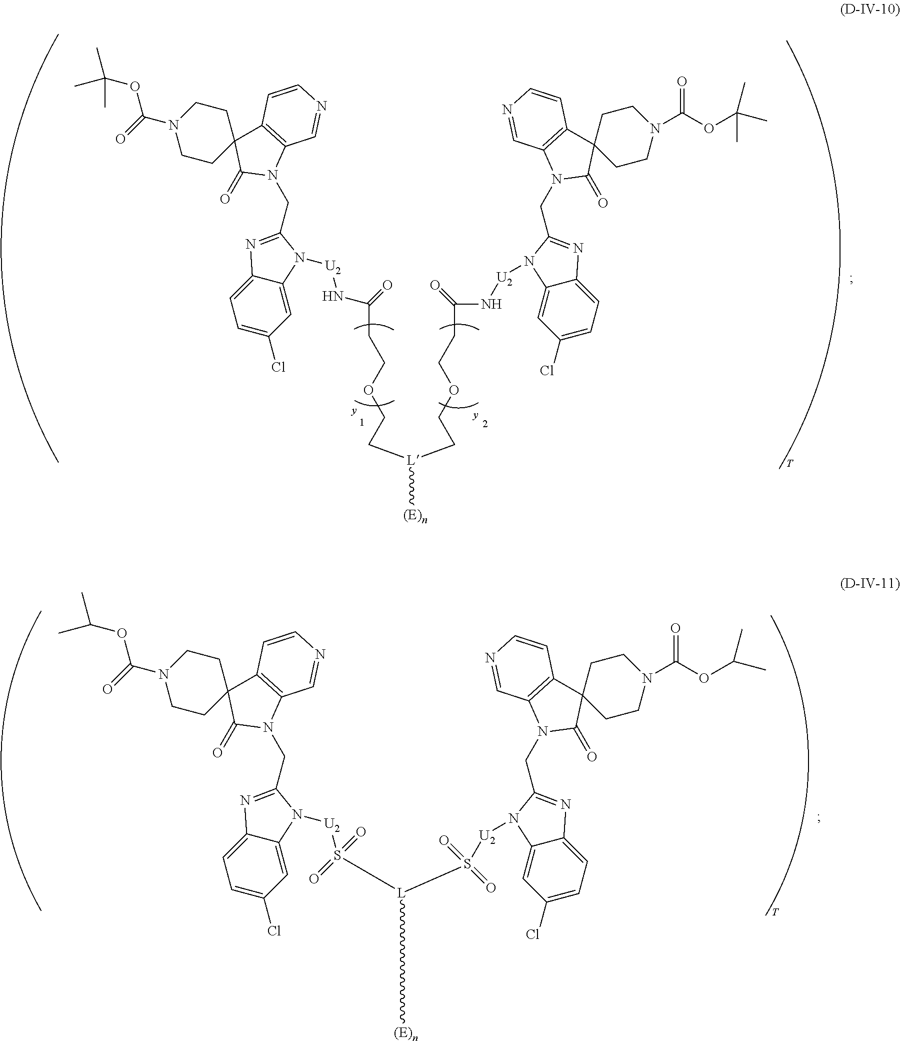

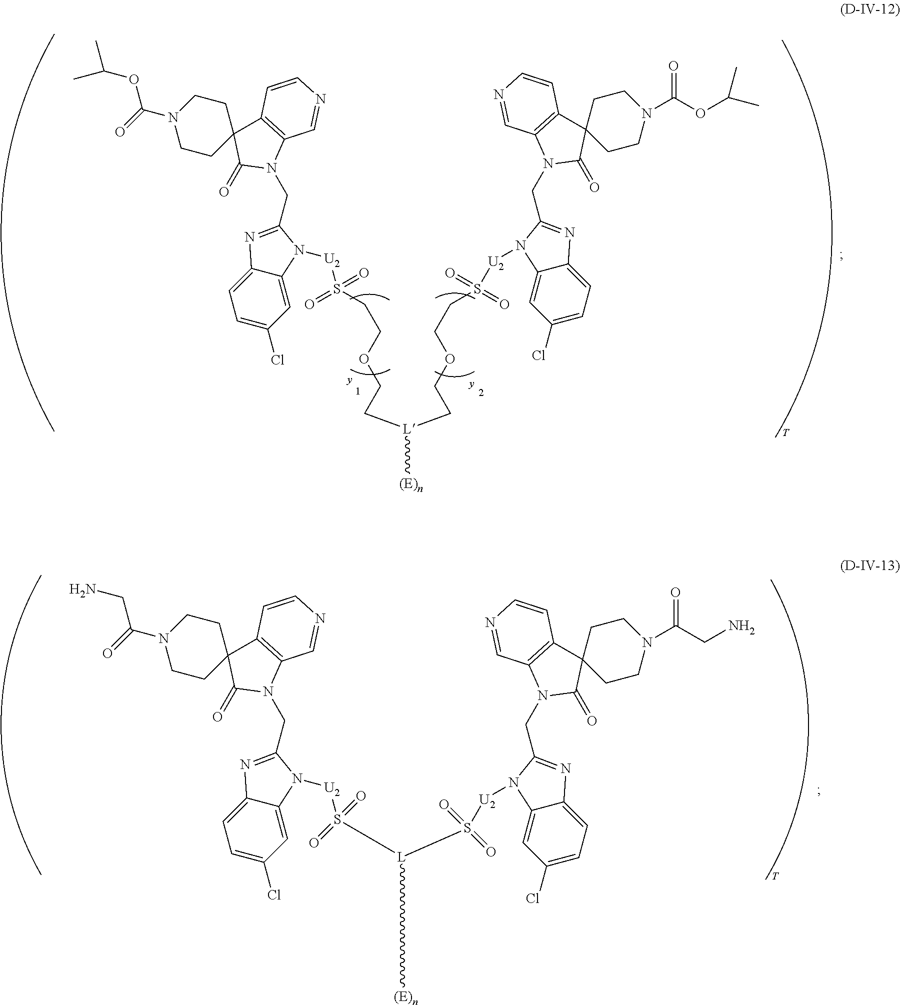

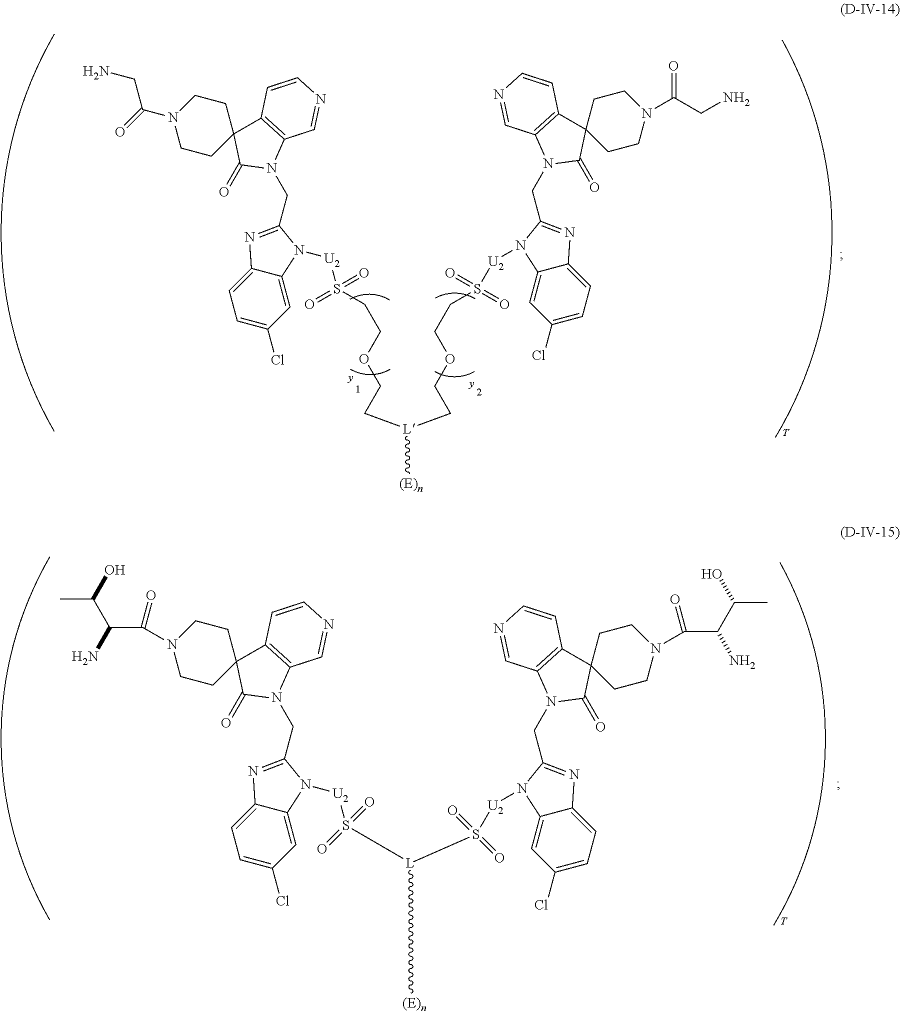

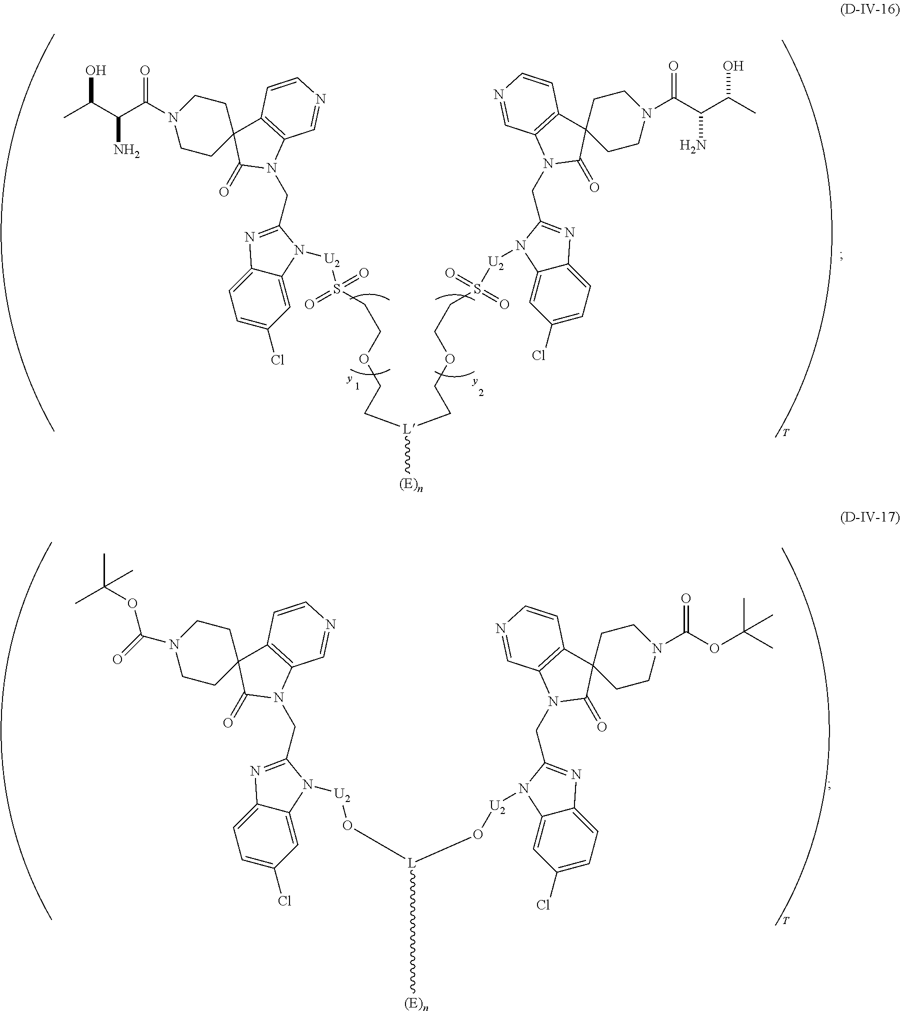

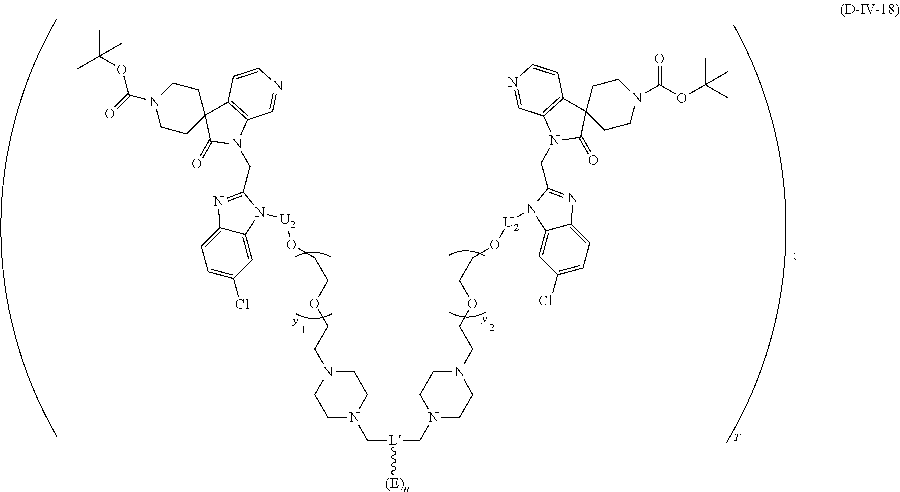

or a pharmaceutically acceptable salt thereof.

In the conjugates described herein, the squiggly line connected to E indicates that one or more (e.g., 1, 2, 3, 4, 5, 6, 7, 8, 9, 10, 11, 12, 13, 14, 15, 16, 17, 18, 19, or 20) dimers of RSV F protein inhibitors may be attached to an Fc domain monomer, Fc domain, Fc-binding peptide, albumin protein, or albumin protein-binding peptide. In some embodiments, when n is 1, one or more (e.g., 1, 2, 3, 4, 5, 6, 7, 8, 9, or 10) dimers of RSV F protein inhibitors may be attached to an Fc domain monomer, Fc domain, Fc-binding peptide, albumin protein, or albumin protein-binding peptide. In some embodiments, when n is 2, one or more (e.g., 1, 2, 3, 4, 5, 6, 7, 8, 9, 10, 11, 12, 13, 14, 15, 16, 17, 18, 19, or 20) dimers of RSV F protein inhibitors may be attached to an Fc domain. The squiggly line in the conjugates described herein is not to be construed as a single bond between one or more dimers of RSV F protein inhibitors and an atom in the Fc domain or albumin protein. In some embodiments, when T is 1, one dimer of RSV F protein inhibitors may be attached to an atom in the Fc domain monomer, Fc domain, Fc-binding peptide, albumin protein, or albumin protein-binding peptide. In some embodiments, when T is 2, two dimers of RSV F protein inhibitors may be attached to an atom in the Fc domain monomer, Fc domain, Fc-binding peptide, albumin protein, or albumin protein-binding peptide.

As described further herein, a linker in a conjugate described herein (e.g., L or L′) may be a branched structure. As described further herein, a linker in a conjugate described herein (e.g., L or L′) may be a multivalent structure, e.g., a divalent or trivalent structure having two or three arms, respectively. In some embodiments when the linker has three arms, two of the arms may be attached to the first and second RSV F protein inhibitors and the third arm may be attached to the Fc domain monomer, Fc domain, Fc-binding peptide, albumin protein, or albumin protein-binding peptide.

In conjugates having an Fc domain covalently linked to one or more dimers of RSV F protein inhibitors, as represented by the formulae above, when n is 2, two Fc domain monomers (each Fc domain monomer is represented by E) dimerize to form an Fc domain.

### Conjugates of Monomers of RSV F Protein Inhibitors Linked to an Fc Domain or an Albumin Protein

In some embodiments, the conjugates described herein include an Fc domain monomer, Fc domain, Fc-binding peptide, albumin protein, or albumin protein-binding peptide covalently linked to one or more monomers of RSV F protein inhibitors. Conjugates of an Fc domain monomer or albumin protein and one or more monomers of RSV F protein inhibitors may be formed by linking the Fc domain or albumin protein to each of the monomers of RSV F protein inhibitors through a linker, such as any of the linkers described herein.

In the conjugates having an Fc domain or albumin protein covalently linked to one or more monomers of RSV F protein inhibitors described herein, the squiggly line connected to E indicates that one or more (e.g., 1, 2, 3, 4, 5, 6, 7, 8, 9, 10, 11, 12, 13, 14, 15, 16, 17, 18, 19, or 20) monomers of RSV F protein inhibitors may be attached to an Fc domain monomer, Fc domain, Fc-binding peptide, albumin protein, or albumin protein-binding peptide. In some embodiments, when n is 1, one or more (e.g., 1, 2, 3, 4, 5, 6, 7, 8, 9, or 10) monomers of RSV F protein inhibitors may be attached to an Fc domain monomer or an albumin protein. In some embodiments, when n is 2, one or more (e.g., 1, 2, 3, 4, 5, 6, 7, 8, 9, 10, 11, 12, 13, 14, 15, 16, 17, 18, 19, or 20) monomers of RSV F protein inhibitors may be attached to an Fc domain. The squiggly line in the conjugates described herein is not to be construed as a single bond between one or more monomers of RSV F protein inhibitors and an atom in the Fc domain monomer, Fc domain, Fc-binding peptide, albumin protein, or albumin protein-binding peptide. In some embodiments, when T is 1, one monomer of RSV F protein inhibitor may be attached to an atom in the Fc domain monomer, Fc domain, Fc-binding peptide, albumin protein, or albumin protein-binding peptide. In some embodiments, when T is 2, two monomers of RSV F protein inhibitors may be attached to an atom in the Fc domain monomer, Fc domain, Fc-binding peptide, albumin protein, or albumin protein-binding peptide.

In some embodiments, when T is greater than 1 (e.g., T is 2, 3, 4, 5, 6, 7, 8, 9, 10, 11, 12, 13, 14, 15, 16, 17, 18, 19, or 20), each A₁-L may be independently selected (e.g., independently selected from any of the A₁-L structures described herein). In some embodiments, E may be conjugated to 2, 3, 4, 5, 6, 7, 8, 9, 10, or more different A₁-L moieties. In some embodiments, E is conjugated to a first A₁-L moiety, and a second A₁-L, moiety. In some embodiments, A₁ of each of the first A₁-L moiety and the second A₁-L moiety is independently selected from any one of formulas (A-I)-(A-III):

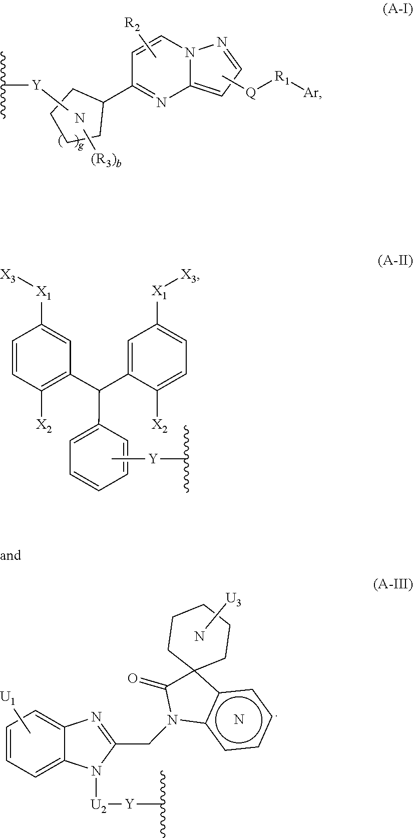

In some embodiments, the first A₁-L moiety is conjugated specifically to lysine residues of E (e.g., the nitrogen atoms of surface exposed lysine residues of E), and the second A₁-L moiety is conjugated specifically to cysteine residues of E (e.g., the sulfur atoms of surface exposed cysteine residues of E). In some embodiments, the first A₁-L moiety is conjugated specifically to cysteine residues of E (e.g., the sulfur atoms of surface exposed cysteine residues of E), and the second A₁-L moiety is conjugated specifically to lysine residues of E (e.g., the nitrogen atoms of surface exposed lysine residues of E).

As described further herein, a linker in a conjugate having an Fc domain monomer, Fc domain, Fc-binding peptide, albumin protein, or albumin protein-binding peptide covalently linked to one or more monomers of the RSV F protein inhibitors described herein (e.g., L or L′) may be a divalent structure having two arms. One arm in a divalent linker may be attached to the monomer of the RSV F protein inhibitor and the other arm may be attached to the Fc domain monomer, Fc domain, Fc-binding peptide, albumin protein, or albumin protein-binding peptide.

In some embodiments, a conjugate containing an Fc domain monomer, Fc domain, Fc-binding peptide, albumin protein, or albumin protein-binding peptide covalently linked to one or more monomers of RSV F protein inhibitor provided herein is described by any one of formulae below:

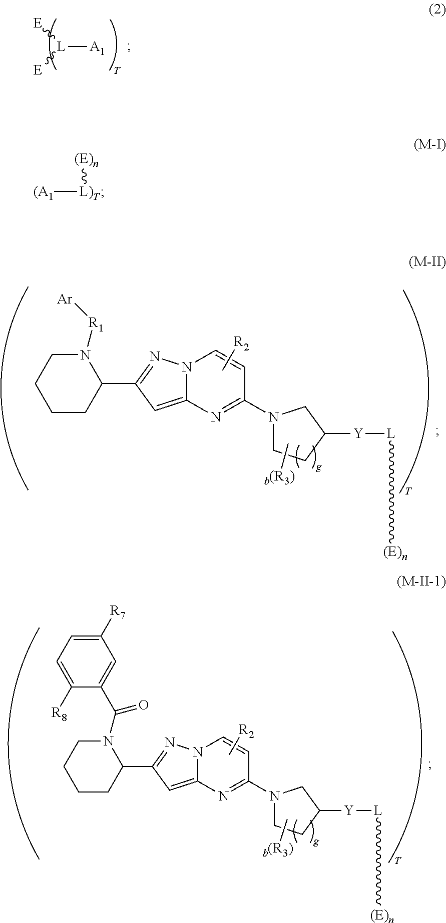

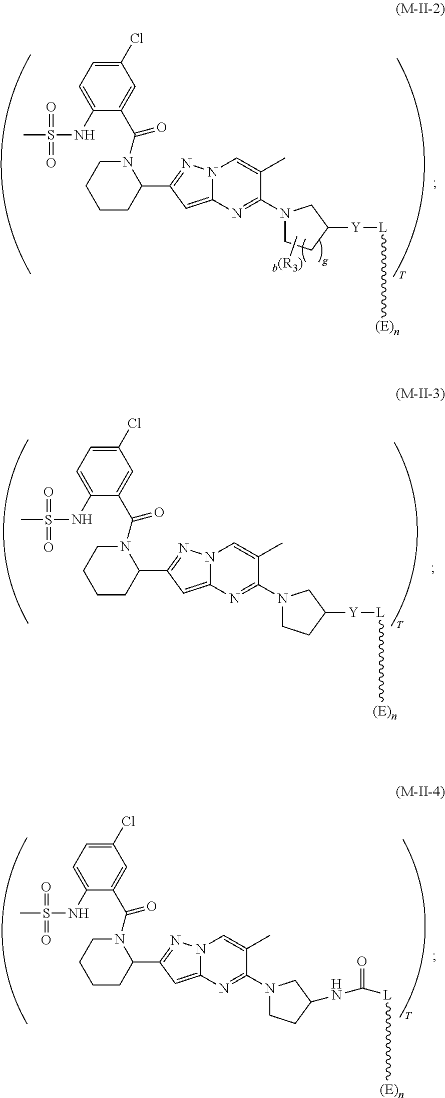

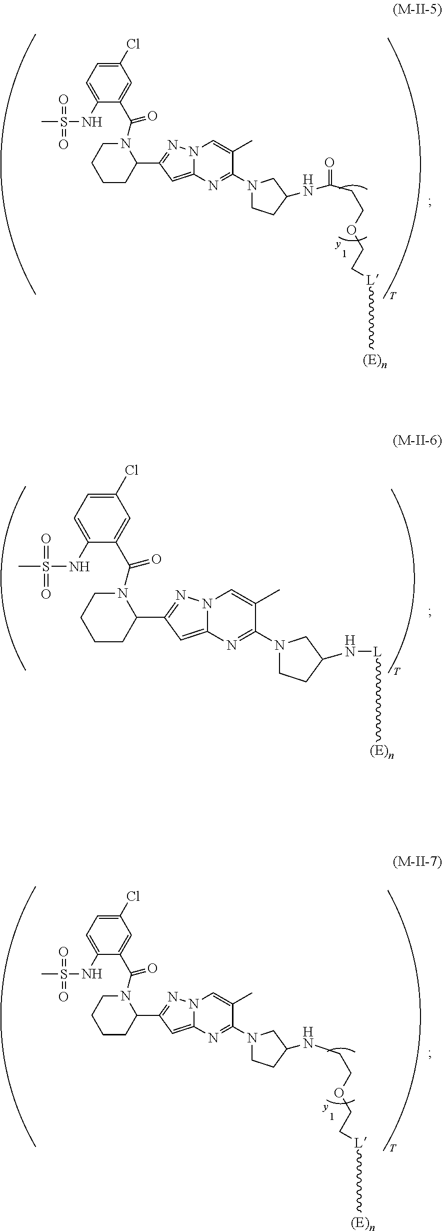

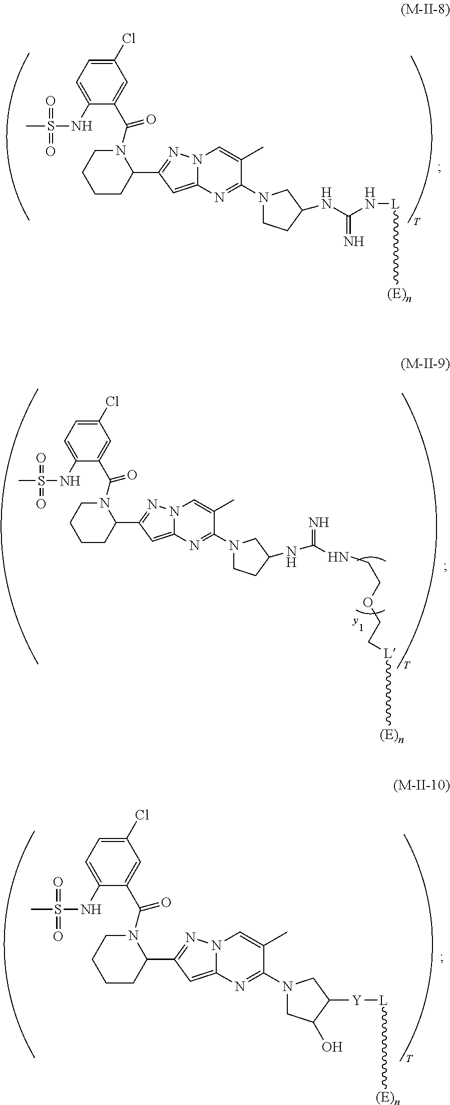

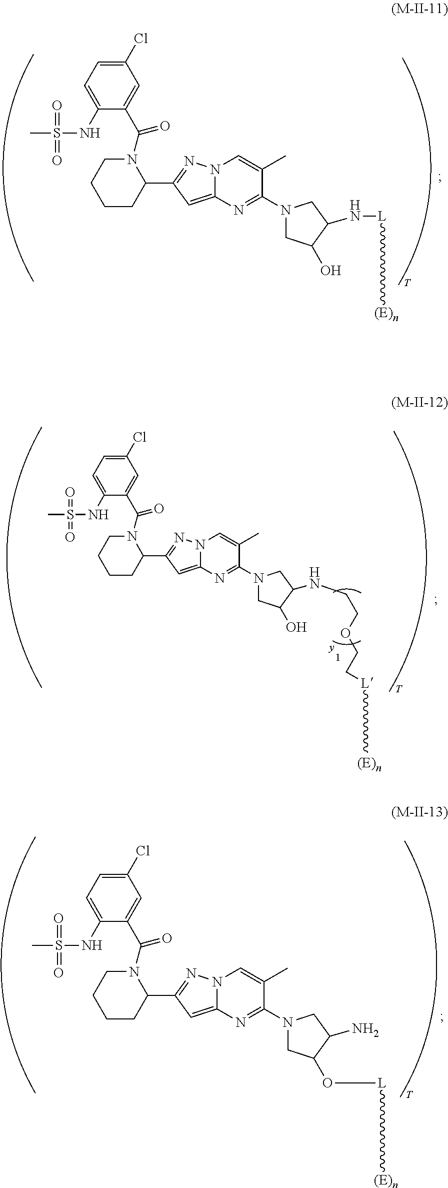

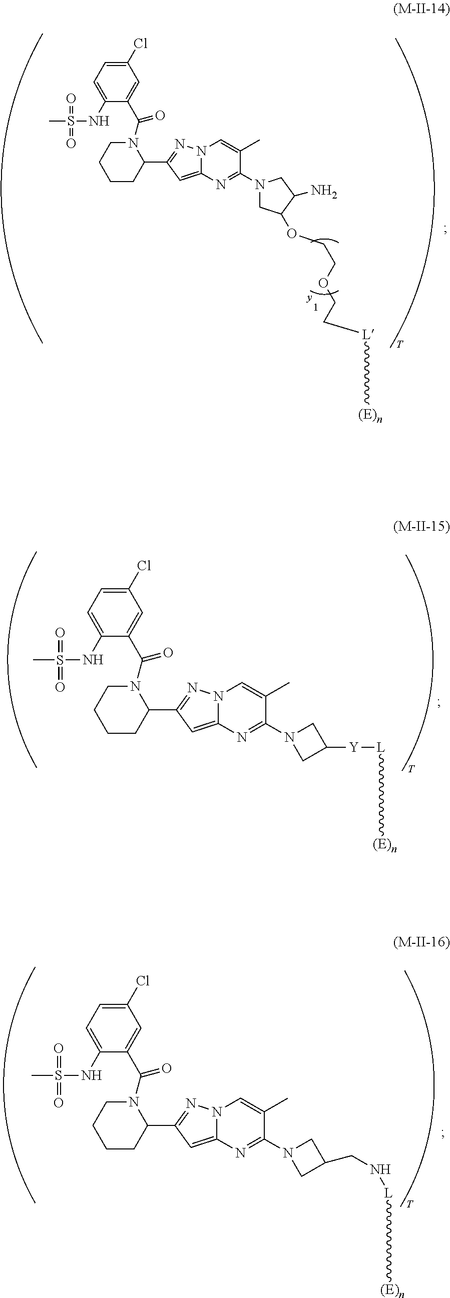

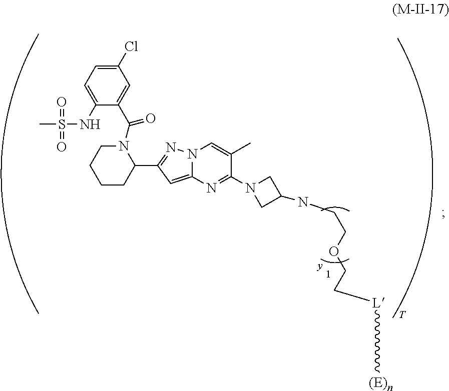

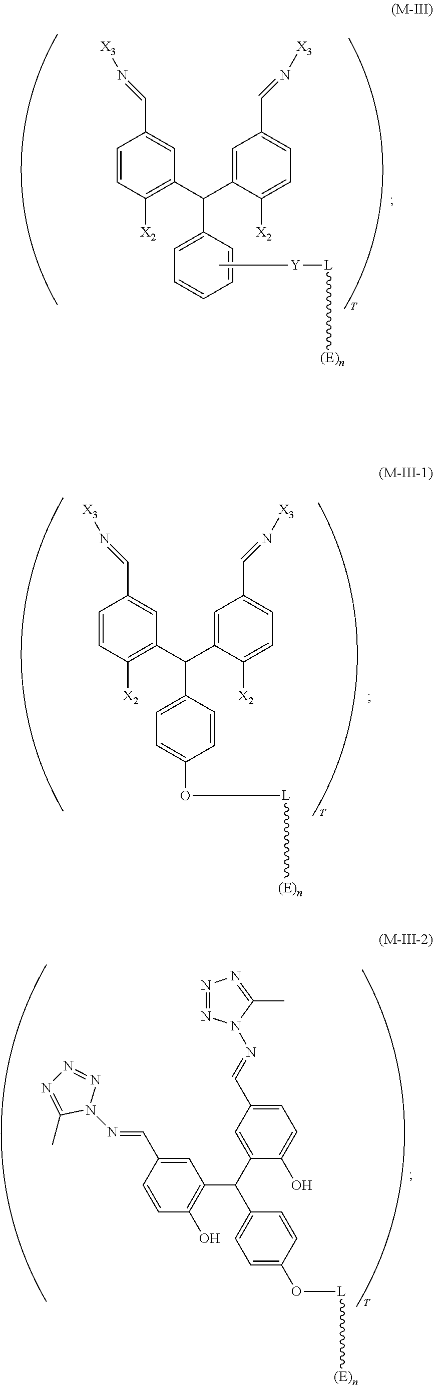

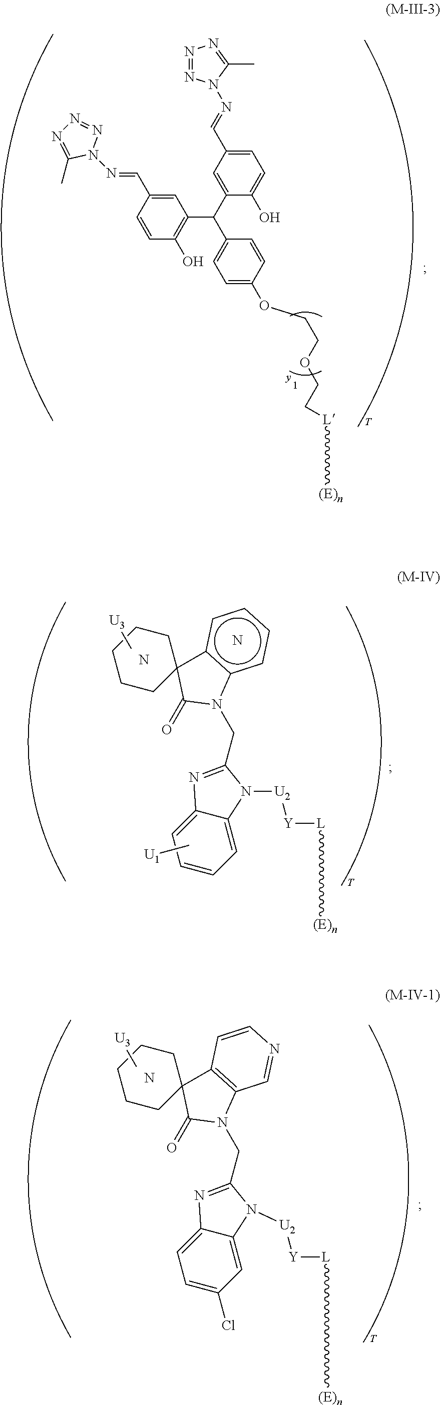

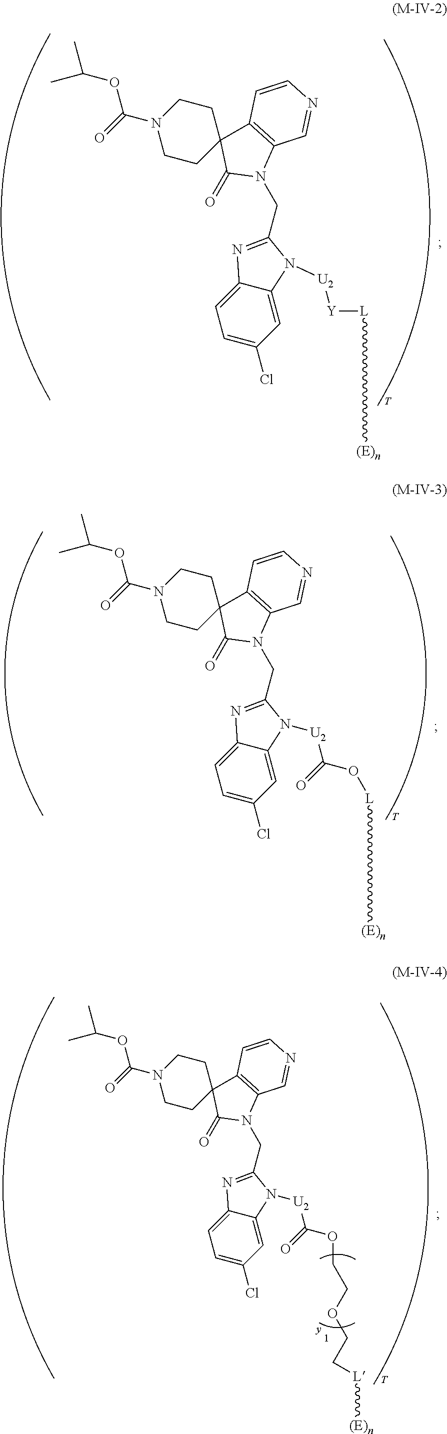

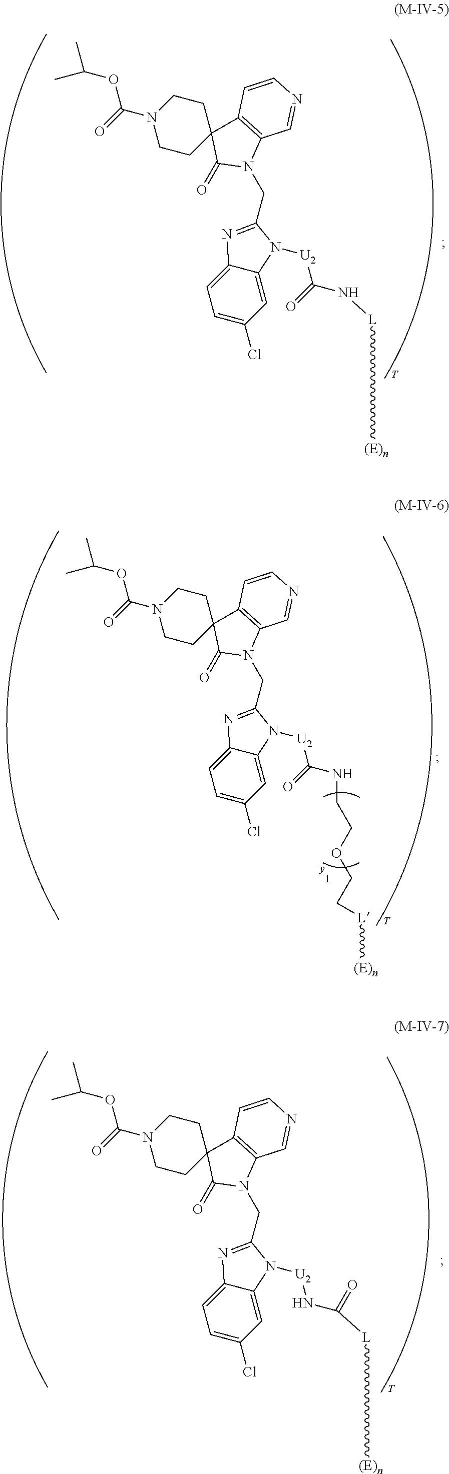

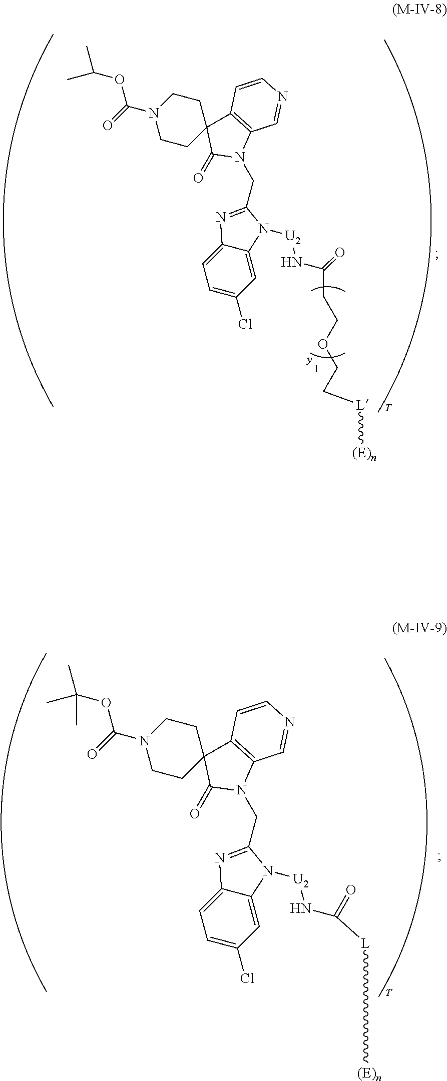

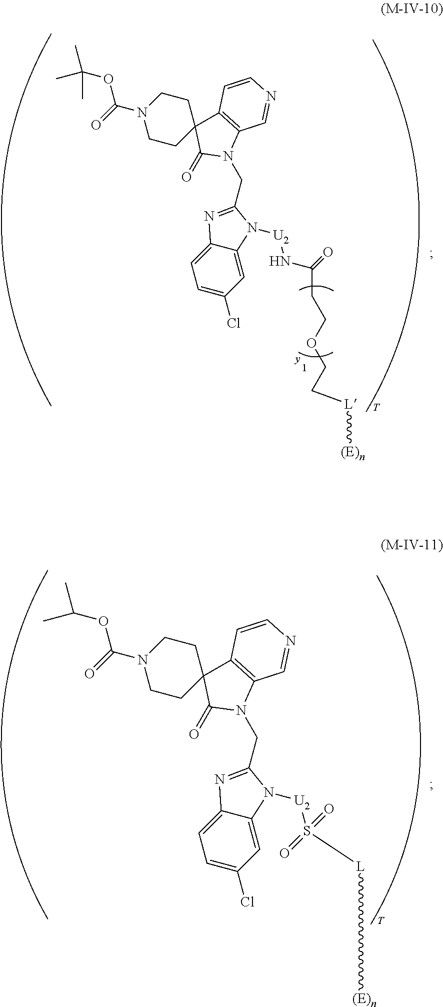

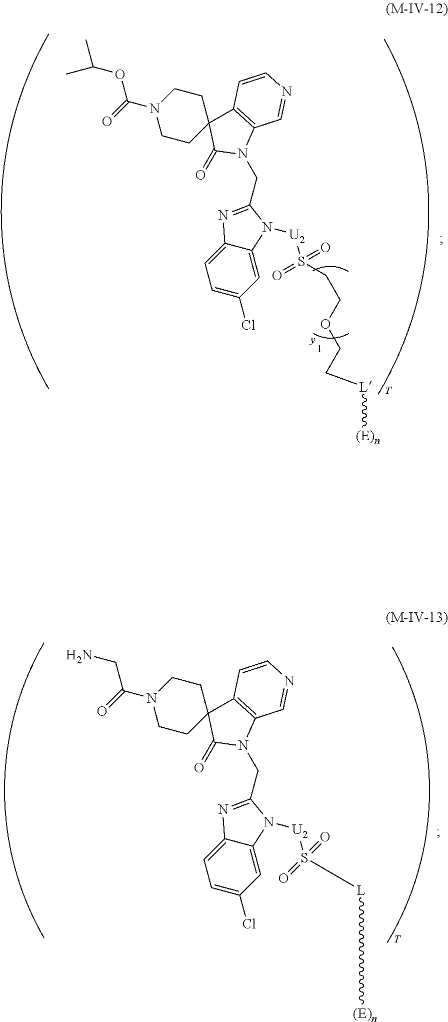

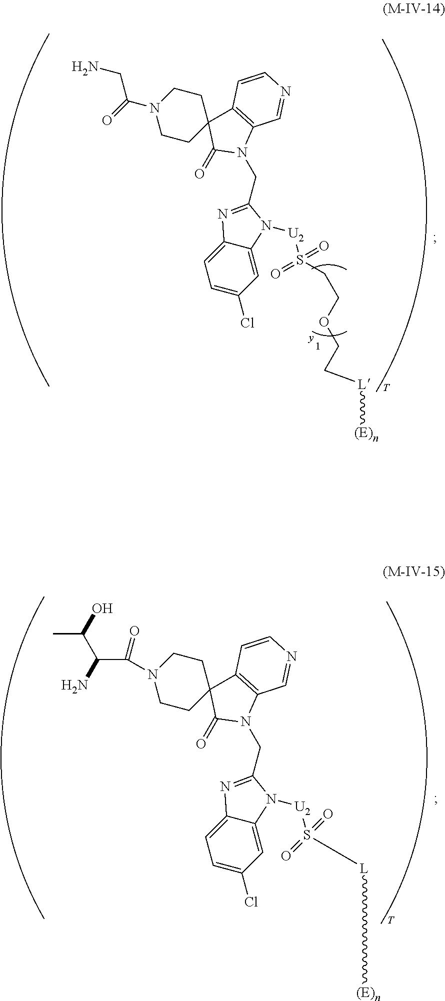

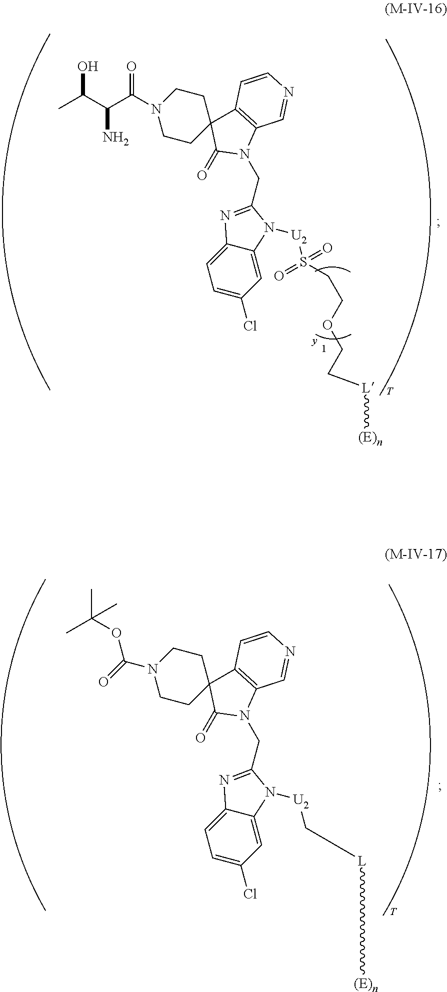

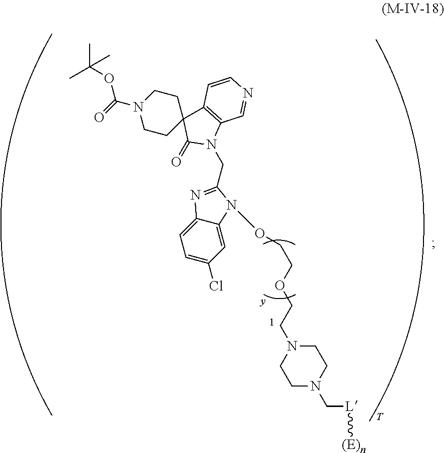

or a pharmaceutically acceptable salt thereof.

In conjugates having an Fc domain covalently linked to one or more monomers of RSV F protein inhibitors, as represented by the formulae above, when n is 2, two Fc domain monomers (each Fc domain monomer is represented by E) dimerize to form an Fc domain.

### III. Fc Domain Monomers and Fc Domains

An Fc domain monomer includes a hinge domain, a C_{H}2 antibody constant domain, and a C_{H}3 antibody constant domain. The Fc domain monomer can be of immunoglobulin antibody isotype IgG, IgE, IgM, IgA, or IgD. The Fc domain monomer can also be of any immunoglobulin antibody isotype (e.g., IgG1, IgG2a, IgG2b, IgG3, or IgG4). The Fc domain monomer can be of any immunoglobulin antibody allotype (e.g., IGHG1*01 (i.e., G1m(za)), IGHG1*07 (i.e., G1m(zax)), IGHG1*04 (i.e., G1m(zav)), IGHG1*03 (G1m(f)), IGHG1*08 (i.e., G1m(fa)), IGHG2*01, IGHG2*06, IGHG2*02, IGHG3*01, IGHG3*05, IGHG3*10, IGHG3*04, IGHG3*09, IGHG3*11, IGHG3*12, IGHG3*06, IGHG3*07, IGHG3*08, IGHG3*13, IGHG3*03, IGHG3*14, IGHG3*15, IGHG3*16, IGHG3*17, IGHG3*18, IGHG3*19, IGHG2*04, IGHG4*01, IGHG4*03, or IGHG4*02) (as described in, for example, in Vidarsson et al. IgG subclasses and allotypes: from structure to effector function. Frontiers in Immunology. 5(520):1-17 (2014)). The Fc domain monomer can also be of any species, e.g., human, murine, or mouse. A dimer of Fc domain monomers is an Fc domain that can bind to an Fc receptor, which is a receptor located on the surface of leukocytes.

In some embodiments, an Fc domain monomer in the conjugates described herein may contain one or more amino acid substitutions, additions, and/or deletion relative to an Fc domain monomer having a sequence of any one of SEQ ID NOs: 1-95. In some embodiments, an Asn in an Fc domain monomer in the conjugates as described herein may be replaced by Ala in order to prevent N-linked glycosylation (see, e.g., SEQ ID NOs: 12-15, where Asn to Ala substitution is labeled with *). In some embodiments, an Fc domain monomer in the conjugates described herein may also containing additional Cys additions (see, e.g., SEQ ID NOs: 9, 10, and 11, where Cys additions are labeled with *).

In some embodiments, an Fc domain monomer in the conjugates as described herein includes an additional moiety, e.g., an albumin-binding peptide, a purification peptide (e.g., a hexa-histidine peptide (HHHHHH (SEQ ID NO: 99)), or a signal sequence (e.g., IL2 signal sequence MYRMQLLSCIALSLALVTNS (SEQ ID NO: 100)) attached to the N- or C-terminus of the Fc domain monomer. In some embodiments, an Fc domain monomer in the conjugate does not contain any type of antibody variable region, e.g., V_{H}, V_{L}, a complementarity determining region (CDR), or a hypervariable region (HVR).

In some embodiments, an Fc domain monomer in the conjugates as described herein may have a sequence that is at least 95% identical (e.g., 97%, 99%, or 99.5% identical) to the sequence of any one of SEQ ID NOs: 1-95 shown below. In some embodiments, an Fc domain monomer in the conjugates as described herein may have a sequence of any one of SEQ ID NOs: 1-95 shown below.

SEQ ID NO: 1: murine Fc-IgG2a with IL2 signal sequence at the N-terminus (bold)
MYRMQLLSCIALSLALVTNSPRGPTIKPCPPCKCPAPNLLGGPSVFIFPPKIKDVLMISLSPIVTCVVVDVS
EDDPDVQISWFVNNVEVHTAQTQTHREDYNSTLRVVSALPIQHQDWMSGKEFKCKVNNKDLPAPIERTI
SKPKGSVRAPQVYVLPPPEEEMTKKQVTLTCMVTDFMPEDIYVEWTNNGKTELNYKNTEPVLDSDGSYF
MYSKLRVEKKNWVERNSYSCSVVHEGLHNHHTTKSFSRTPGK
SEQ ID NO: 2: mature murine Fc-IgG2a
PRGPTIKPCPPCKCPAPNLLGGPSVFIFPPKIKDVLMISLSPIVTCVVVDVSEDDPDVQISWFVNNVEVHTA
QTQTHREDYNSTLRVVSALPIQHQDWMSGKEFKCKVNNKDLPAPIERTISKPKGSVRAPQVYVLPPPEEE
MTKKQVTLTCMVTDFMPEDIYVEWTNNGKTELNYKNTEPVLDSDGSYFMYSKLRVEKKNWVERNSYSC
SVVHEGLHNHHTTKSFSRTPGK
SEQ ID NO: 3: human Fc-IgG1 with IL2 signal sequence at the N-terminus (bold) and N-terminal MVRS
amino acid residues added (underlined)
MYRMQLLSCIALSLALVTNSMVRSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 4: mature human Fc-IgG1 with N-terminal MVRS amino acid residues added (underlined)
MVRSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKSRWQQGNVF
SCSVMHEALHNHYTQKSLSLSPGK
SEQ ID NO: 5: murine Fc-IgG2a with IL2 signal sequence (bold) at the N-terminus and hexa-histidine
peptide (italicized) at the C-terminus
MYRMQLLSCIALSLALVTNSPRGPTIKPCPPCKCPAPNLLGGPSVFIFPPKIKDVLMISLSPIVTCVVVDVS
EDDPDVQISWFVNNVEVHTAQTQTHREDYNSTLRVVSALPIQHQDWMSGKEFKCKVNNKDLPAPIERTI
SKPKGSVRAPQVYVLPPPEEEMTKKQVTLTCMVTDFMPEDIYVEWTNNGKTELNYKNTEPVLDSDGSYF
MYSKLRVEKKNWVERNSYSCSVVHEGLHNHHTTKSFSRTPGKHHHHHH
SEQ ID NO: 6: mature murine Fc-IgG2a with hexa-histidine peptide (italicized) at the C-terminus
PRGPTIKPCPPCKCPAPNLLGGPSVFIFPPKIKDVLMISLSPIVTCVVVDVSEDDPDVQISWFVNNVEVHTA
QTQTHREDYNSTLRVVSALPIQHQDWMSGKEFKCKVNNKDLPAPIERTISKPKGSVRAPQVYVLPPPEEE
MTKKQVTLTCMVTDFMPEDIYVEWTNNGKTELNYKNTEPVLDSDGSYFMYSKLRVEKKNWVERNSYSC
SVVHEGLHNHHTTKSFSRTPGKHHHHHH
SEQ ID NO: 7: human Fc-IgG1 with IL2 signal sequence (bold) at the N-terminus, N-terminal MVRS
amino acid residues added (underlined), and hexa-histidine peptide (italicized) at the C-terminus
MYRMQLLSCIALSLALVTNSMVRSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGKHHHHHH
SEQ ID NO: 8: mature human Fc-IgG1 with hexa-histidine peptide (italicized) at the C-terminus and N-
terminal MVRS amino acid residues added (underlined)
MVRSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKSRWQQGNVF
SCSVMHEALHNHYTQKSLSLSPGKHHHHHH
SEQ ID NO: 9: human Fc-IgG1 with IL2 signal sequence (bold) at the N-terminus, N-terminal MVRS
amino acid residues added (underlined), two additional cysteines in the hinge region (*), and hexa-
histidine peptide (italicized) at the C-terminus
MYRMQLLSCIALSLALVTNSMVRSDKTHTCPPCPPC*KC*PAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGKHHHHHH
SEQ ID NO: 10: mature human Fc-IgG1 with N-terminal MVRS amino acid residues added (underlined),
two additional cysteines in the hinge region (*), and hexa-histidine peptide (italicized) at the C-
terminus
MVRSDKTHTCPPCPPC*KC*PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKHHHHHH
SEQ ID NO: 11: mature human Fc-IgG1 with N-terminal MVRS amino acid residues added (underlined)
and two additional cysteines in the hinge region (*)
MVRSDKTHTCPPCPPC*KC*PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID NO: 12: murine Fc-IgG2a with IL2 signal sequence (bold) at the N-terminus, Asn to Ala
substitution (*), and hexa-histidine peptide (italicized) at the C-terminus
MYRMQLLSCIALSLALVTNSPRGPTIKPCPPCKCPAPNLLGGPSVFIFPPKIKDVLMISLSPIVTCVVVDVS
EDDPDVQISWFVNNVEVHTAQTQTHREDYA*STLRVVSALPIQHQDWMSGKEFKCKVNNKDLPAPIERTI
SKPKGSVRAPQVYVLPPPEEEMTKKQVTLTCMVTDFMPEDIYVEWTNNGKTELNYKNTEPVLDSDGSYF
MYSKLRVEKKNWVERNSYSCSVVHEGLHNHHTTKSFSRTPGKHHHHHH
SEQ ID NO: 13: mature murine Fc-IgG2a with Asn to Ala substitution (*) and hexa-histidine peptide
(italicized) at the C-terminus
PRGPTIKPCPPCKCPAPNLLGGPSVFIFPPKIKDVLMISLSPIVTCVVVDVSEDDPDVQISWFVNNVEVHTA
QTQTHREDYA*STLRVVSALPIQHQDWMSGKEFKCKVNNKDLPAPIERTISKPKGSVRAPQVYVLPPPEE
EMTKKQVTLTCMVTDFMPEDIYVEWTNNGKTELNYKNTEPVLDSDGSYFMYSKLRVEKKNWVERNSYS
CSVVHEGLHNHHTTKSFSRTPGKHHHHHH
SEQ ID NO: 14: human Fc-IgG1 with IL2 signal sequence (bold) at the N-terminus, N-terminal MVRS
amino acid residues added (underlined), Asn to Ala substitution (*), and hexa-histidine peptide
(italicized) at the C-terminus
MYRMQLLSCIALSLALVTNSMVRSDKTHTCPPCPAPELLGGPSVFLFPPKPKDTLMISRTPEVTCVVVDV
SHEDPEVKFNWYVDGVEVHNAKTKPREEQYA*STYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGKHHHHHH
SEQ ID NO: 15: mature human Fc-IgG1 with Asn to Ala substitution (*), N-terminal MVRS amino acid
residues added (underlined), and hexa-histidine peptide (italicized) at the C-terminus
MVRSDKTHTCPPCPAPELLGGPSVFLFPPKPKDTLMISRTPEVTCVVVDVSHEDPEVKFNWYVDGVEVH
NAKTKPREEQYA*STYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDKSRWQQGNV
FSCSVMHEALHNHYTQKSLSLSPGKHHHHHH
SEQ ID NO: 16: human IgG1 Fc with Human Serum Albumin Signal Sequence (bold) at the N-terminus
and N-terminal ISAMVRS amino acid residues added (underlined)
MKWVTFISLLFLFSSAYSISAMVRSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 17: human IgG1 Fc with Human Serum Albumin Signal Sequence (bold) at the N-terminus,
N-terminal ISAMVRS amino acid residues added (underlined), C-terminal G4S linker (italicized), and C-
terminal c-Myc tag (underlined, italicized)
MKWVTFISLLFLFSSAYSISAMVRSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGGGGGSEQKLISEEDL
SEQ ID NO: 18: mature human IgG1 Fc with N-terminal ISAMVRS amino acid residues added
(underlined), C-terminal G4S linker (italicized), and C-terminal c-Myc tag (underlined, italicized)
ISAMVRSDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSEQKLISEEDL
SEQ ID NO: 19: human IgG1 Fc with Human Serum Albumin Signal Sequence (bold), N-terminal
ISAMVRS amino acid residues added (underlined), and lysine to serine modification (*) to prevent lysine
conjugation at this site
MKWVTFISLLFLFSSAYSISAMVRSDKTHTCPPCPAPELLGGPSVFLFPPKPS*DTLMISRTPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 20: mature human IgG1 Fc with N-terminal ISAMVRS amino acid residues added
(underlined) and lysine to serine modification (*) to prevent lysine conjugation at this site
ISAMVRSDKTHTCPPCPAPELLGGPSVFLFPPKPS*DTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
SEQ ID NO: 21: human IgG1 Fc with Human Serum Albumin Signal Sequence (bold) at the N-terminus,
N-terminal ISAMVRS amino acid residues added (underlined), lysine to serine modification (*) to prevent
lysine conjugation at this site, C-terminal G4S linker (italicized), and C-terminal C-Myc tag
(underlined, italicized)
MKWVTFISLLFLFSSAYSISAMVRSDKTHTCPPCPAPELLGGPSVFLFPPKPS(*)DTLMISRTPEVTCVVV
DVSHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGGGGGSEQKLISEEDL
SEQ ID NO: 22: mature human IgG1 Fc with N-terminal ISAMVRS amino acid residues added
(underlined), lysine to serine modification (*) to prevent lysine conjugation at this site, C-terminal
G4S linker (italicized), and C-terminal C-Myc tag (underlined, italicized)
ISAMVRSDKTHTCPPCPAPELLGGPSVFLFPPKPS(*)DTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSEQKLISEEDL
SEQ ID NO: 23: human IgG1 Fc with Human Serum Albumin Signal Sequence (bold) at the N-terminus,
N-terminal ISAMVRS amino acid residues added (underlined), Asn to Ala substitution (*), C-terminal G4S
linker (italicized), and C-terminal C-myc tag (underlined, italicized)
MKWVTFISLLFLFSSAYSISAMVRSDKTHTCPPCPAPELLGGPSVFLFPPKPKDTLMISRTPEVTCVVVDV
SHEDPEVKFNWYVDGVEVHNAKTKPREEQYA(*)STYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGGGGGSEQKLISEEDL
SEQ ID NO: 24: mature human IgG1 Fc with N-terminal ISAMVRS amino acid residues added
(underlined), Asn to Ala substitution (*), C-terminal G4S linker (italicized), and C-terminal C-myc tag
(underlined, italicized)
ISAMVRSDKTHTCPPCPAPELLGGPSVFLFPPKPKDTLMISRTPEVTCVVVDVSHEDPEVKFNWYVDGVE
VHNAKTKPREEQYA(*)STYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSEQKLISEEDL
SEQ ID NO: 25: human IgG1 Fc with Human Serum Albumin Signal Sequence (bold) at the N-terminus,
N-terminal ISAMVRS amino acid residues added (underlined), H310A (*) and H435A (*) mutations to
impede FcRn binding, C-terminal G4S (italicized), and C-terminal C-myc tag (underlined, italicized)
MKWVTFISLLFLFSSAYSISAMVRSDKTHTCPPCPAPELLGGPSVFLFPPKPKDTLMISRTPEVTCVVVDV
SHEDPEVKFNWYVDGVEVHNAKTKPREEQYNSTYRVVSVLTVLA(*)QDWLNGKEYKCKVSNKALPAPIE
KTISKA(*)KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNAYTQKSLSLSPGGGGGSEQKLISEEDL
SEQ ID NO: 26: mature human IgG1 Fc with Human Serum Albumin Signal Sequence (bold) at the N-
terminus, N-terminal ISAMVRS amino acid residues added (underlined), with H310A (*) and H435A (*)
mutations to impede FcRn binding, C-terminal G4S (italicized), and C-terminal C-myc tag (underlined,
italicized)
ISAMVRSDKTHTCPPCPAPELLGGPSVFLFPPKPKDTLMISRTPEVTCVVVDVSHEDPEVKFNWYVDGVE
VHNAKTKPREEQYNSTYRVVSVLTVLA(*)QDWLNGKEYKCKVSNKALPAPIEKTISKA(*)KGQPREPQVY
TLPPSREEMTKNQVSLTCLVKGFYPSDIAVEWESNGQPENNYKTTPPVLDSDGSFFLYSKLTVDKSRWQ
QGNVFSCSVMHEALHNAYTQKSLSLSPGGGGGSEQKLISEEDL
SEQ ID NO: 27: human IgG1 Fc with Human Serum Albumin Signal Sequence (bold) at the N-terminus,
N-terminal ISAMVRS amino acid residues added (underlined), C-terminal G4S linker (italicized), and C-
terminal mutated (lysine to phenylalanine, bold) C-myc tag (underlined, italicized)
MKWVTFISLLFLFSSAYSISAMVRSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGGGGGSEQ [symbol] LISEEDL
SEQ ID NO: 28: mature human IgG1 Fc with N-terminal ISAMVRS amino acid residues added
(underlined), C-terminal G4S linker (italicized), and C-terminal mutated (lysine to phenylalanine, bold)
C-myc tag (underlined, italicized)
ISAMVRSDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSEQ [symbol] LISEEDL
SEQ ID NO: 29: human IgG1 Fc with Human Serum Albumin Signal Sequence (bold) at the N-terminus,
N-terminal ISAMVRS amino acid residues added (underlined), Asn to Ala substitution (*), C-terminal G4S
linker (italicized), and C-terminal mutated (lysine to phenylalanine, bold) C-myc tag (underlined,
italicized)
MKWVTFISLLFLFSSAYSISAMVRSDKTHTCPPCPAPELLGGPSVFLFPPKPKDTLMISRTPEVTCVVVDV
SHEDPEVKFNWYVDGVEVHNAKTKPREEQYA(*)STYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGGGGGSEQFLISEEDL
SEQ ID NO: 30: mature human IgG1 Fc with N-terminal MVRS amino acid residues added (underlined),
Asn to Ala substitution (*), C-terminal G4S linker (italicized), and C-terminal mutated (lysine to
phenylalanine, bold) C-myc tag (underlined, italicized)
ISAMVRSDKTHTCPPCPAPELLGGPSVFLFPPKPKDTLMISRTPEVTCVVVDVSHEDPEVKFNWYVDGVE
VHNAKTKPREEQYA(*)STYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSEQ [symbol] LISEEDL
SEQ ID NO: 31: human IgG1 Fc with Human Serum Albumin Signal Sequence (bold) at the N-terminus,
allotype G1m(fa)(bold italics), C-terminal G4S linker (italicized), and C-terminal mutated (lysine to
phenylalanine, bold) C-myc tag (underlined)
MKWVTFISLLFLFSSAYSDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVYTLPPSR [symbol] E [symbol] TKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSEQFLISEEDL
SEQ ID NO: 32: human IgG1 Fc with Human Serum Albumin Signal Sequence (bold) at the N-terminus,
allotype G1m(fa)(bold italics)
MKWVTFISLLFLFSSAYSDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVYTLPPSR [symbol] E [symbol] TKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 33: mature human IgG1 Fc with a YTE triple mutation (bold and underlined) with N-terminal
MVRS amino acid residues added (underlined)
MVRSDKTHTCPPCPAPELLGGPSVFLFPPKPKDTLYITREPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKSRWQQGNVF
SCSVMHEALHNHYTQKSLSLSPGK
SEQ ID NO: 34: human IgG1 Fc with Human Serum Albumin Signal Sequence (bold) at the N-terminus,
contains residues EPKSS including the full hinge region on the N-terminus of mature human IgG1 Fc
(underlined), Cys to Ser substitution (#), allotype G1m(fa)(bold italics)
MKWVTFISLLFLFSSAYSEPKSS(#)DKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSR [symbol] E [symbol] TKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 35: human IgG1 Fc with murine IgG signal sequence (bold) at the N-terminus, with removal
of EPKSSD hinge residues from the N-terminus of the mature human IgG1 Fc, allotype G1m(fa)(bold
italics)
MGWSCIILFLVATATGVHSKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVYTLPPSR [symbol] E [symbol] TKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 36: mature human IgG1 Fc with a YTE triple mutation (bold and underlined), with removal of
EPKSSD hinge residues from the N-terminus of the mature human IgG1 Fc, allotype G1m(fa)(bold
italics)
KTHTCPPCPAPELLGGPSVFLFPPKPKDTLYITREPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSR [symbol] E [symbol]
TKNQVSLTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 37: mature human IgG1 Fc with an LS double mutation (bold and underlined), with removal
of EPKSSD hinge residues from the N-terminus of the mature human IgG1 Fc, allotype G1m(fa)(bold
italics)
KTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSR [symbol] E [symbol]
TKNQVSLTCLVKGFYPSDIAVEWESNGQPENNYKTTPPVLDSDGSFFLYSKLTVDKSRWQQGNVFSCS
VLHEALHSHYTQKSLSLSPGK
SEQ ID NO: 38: mature human IgG1 Fc with Human Serum Albumin Signal Sequence (bold) at the N-
terminus, a YTE triple mutation (bold and underlined), allotype G1m(fa)(bold italics), C-terminal G4S
linker (italicized), and C-terminal C-myc tag (underlined)
MKWVTFISLLFLFSSAYSDKTHTCPPCPAPELLGGPSVFLFPPKPKDTLYITREPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSR [symbol] E [symbol] TKNQVSLTCLVKGFYPSDIAVEWESNGQPENNYKTTPPVLDSDGSFFLYSKLTV
DKSRWQQGNVFSCSVMHEALHNHYTQKSLSLSPGGGGGSEQKLISEEDL
SEQ ID NO: 39: mature human Fc IgG1, wherein X₁ is Met or Trp, X₂ is Ser or Thr, X₃ is Thr or Glu, X₄ is
Asp or Glu, and X₅ is Leu or Met, X₆ is Met or Leu, and X₇ is Asn or Ser
DKTHTCPPCPAPELLGGPSVFLFPPKPKDTL [symbol] R [symbol] PEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSR [symbol] E [symbol]
TKNQVSLTCLVKGFYPSDIAVEWESNGQPENNYKTTPPVLDSDGSFFLYSKLTVDKSRWQQGNVFSC
SV [symbol] HEALH [symbol] HYTQKSLSLSPG
SEQ ID NO: 40: mature human Fc IgG1 wherein X₄ is Asp or Glu, and X₅ is Leu or Met
DKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSR [symbol] E [symbol]
TKNQVSLTCLVKGFYPSDIAVEWESNGQPENNYKTTPPVLDSDGSFFLYSKLTVDKSRWQQGNVFSCSV
MHEALHNHYTQKSLSLSPG
SEQ ID NO: 41: mature human Fc IgG1 with a YTE triple mutation (bold and underlined), and wherein X₄
is Asp or Glu, and X₅ is Leu or Met
DKTHTCPPCPAPELLGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSR [symbol] E [symbol] T
KNQVSLTCLVKGFYPSDIAVEWESNGQPENNYKTTPPVLDSDGSFFLYSKLTVDKSRWQQGNVFSCSV
MHEALHNHYTQKSLSLSPG
SEQ ID NO: 42: mature human Fc IgG1 with a YTE triple mutation (bold and underlined), allotype
G1m(fa)(bold italics)
DKTHTCPPCPAPELLGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSR [symbol] E [symbol] TK
NQVSLTCLVKGFYPSDIAVEWESNGQPENNYKTTPPVLDSDGSFFLYSKLTVDKSRWQQGNVFSCSVM
HEALHNHYTQKSLSLSPG
SEQ ID NO: 43: mature human Fc IgG1 with a YTE triple mutation (bold and underlined), allotype G1m(f)
(bold italics)
DKTHTCPPCPAPELLGGPSVFLFPPKPKDTLYITREPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSR [symbol] E [symbol] TK
NQVSLTCLVKGFYPSDIAVEWESNGQPENNYKTTPPVLDSDGSFFLYSKLTVDKSRWQQGNVFSCSVM
HEALHNHYTQKSLSLSPG
SEQ ID NO: 44: mature human Fc IgG1 with a LS double mutation (bold and underlined), and wherein X₄
is Asp or Glu, and X₅ is Leu or Met
DKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSR [symbol] E [symbol]
TKNQVSLTCLVKGFYPSDIAVEWESNGQPENNYKTTPPVLDSDGSFFLYSKLTVDKSRWQQGNVFSCSV
LHEALHSHYTQKSLSLSPG
SEQ ID NO: 45: mature human Fc IgG1 with a LS double mutation (bold and underlined), allotype
G1m(fa)(bold italics)
DKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSR [symbol] E [symbol] T
KNQVSLTCLVKGFYPSDIAVEWESNGQPENNYKTTPPVLDSDGSFFLYSKLTVDKSRWQQGNVFSCSVL
HEALHSHYTQKSLSLSPG
SEQ ID NO: 46: mature human Fc IgG1 with a LS double mutation (bold and underlined), allotype G1m(f)
(bold italics)
DKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSR [symbol] E [symbol] T
KNQVSLTCLVKGFYPSDIAVEWESNGQPENNYKTTPPVLDSDGSFFLYSKLTVDKSRWQQGNVFSCSVL
HEALHSHYTQKSLSLSPG
SEQ ID NO: 47: mature human Fc IgG1 with mouse heavy chain MIgG Vh signal sequence (bold),
deletion of Asp (Cys to Ser substitution (#), and wherein X₁ is Met or Trp, X₂ is Ser or Thr, X₃ is Thr
or Glu, X₄ is Asp or Glu, and X₅ is Leu or Met, X₆ is Met or Leu, and X₇ is Asn or Ser
MGWSCIILFLVATATGVHSNVNHKPSNTKVDKKVEPKSS(#)DKTHTCPPCPAPELLGGPSVFLFPPKPKD
TL [symbol] R [symbol] PEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSR [symbol] E [symbol] TKNQVSLTCLVKGFYPSDIAVEWESNG
QPENNYKTTPPVLDSDGSFFLYSKLTVDKSRWQQGNVFSCSV [symbol] HEALH [symbol] HYTQKSLSLSPG
SEQ ID NO: 48: mature human IgG1 Fc with mouse heavy chain MIgG Vh signal sequence (bold), Cys to
Ser substitution (#), allotype G1m(fa)(bold italics)
MGWSCIILFLVATATGVHSNVNHKPSNTKVDKKVEPKSS(#)DKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSR [symbol] E [symbol] TKNQVSLTCLVKGFYPSDIAVEWESNGQP
ENNYKTTPPVLDSDGSFFLYSKLTVDKSRWQQGNVFSCSVMHEALHNHYTQKSLSLSPGK
SEQ ID NO: 49: mature human IgG1 Fc with mouse heavy chain MIgG Vh signal sequence (bold), Cys to
Ser substitution (#), allotype G1m(f)(bold italics)
MGWSCIILFLVATATGVHSNVNHKPSNTKVDKKVEPKSS(#)DKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSR [symbol] E [symbol] TKNQVSLTCLVKGFYPSDIAVEWESNGQP
ENNYKTTPPVLDSDGSFFLYSKLTVDKSRWQQGNVFSCSVMHEALHNHYTQKSLSLSPGK
SEQ ID NO: 50: mature human IgG1 Fc with mouse heavy chain MIgG Vh signal sequence (bold), Cys to
Ser substitution (#), M428L, N434S mutations (Bold/Underlined), allotype G1m(fa)(bold italics)
MGWSCIILFLVATATGVHSNVNHKPSNTKVDKKVEPKSS(#)DKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSR [symbol] E [symbol] TKNQVSLTCLVKGFYPSDIAVEWESNGQP
ENNYKTTPPVLDSDGSFFLYSKLTVDKSRWQQGNVFSCSVLHEALHSHYTQKSLSLSPGK
SEQ ID NO: 51: mature human IgG1 Fc with mouse heavy chain MIgG Vh signal sequence (bold), Cys
to Ser substitution (#), M428L, N434S mutations (Bold/Underlined), allotype G1m(f)(bold italics)
MGWSCIILFLVATATGVHSNVNHKPSNTKVDKKVEPKSS(#)DKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSR [symbol] E [symbol] TKNQVSLTCLVKGFYPSDIAVEWESNGQP
ENNYKTTPPVLDSDGSFFLYSKLTVDKSRWQQGNVFSCSVLHEALHSHYTQKSLSLSPGK
SEQ ID NO: 52: mature human IgG1 Fc with mouse heavy chain MIgG Vh signal sequence (bold), Cys to
Ser substitution (#), YTE triple mutation (bold and underlined), allotype G1m(fa)(bold italics)
MGWSCIILFLVATATGVHSNVNHKPSNTKVDKKVEPKSS(#)DKTHTCPPCPAPELLGGPSVFLFPPKPKD
TLYITREPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSR [symbol] E [symbol] TKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 53: mature human IgG1 Fc with mouse heavy chain MIgG Vh signal sequence (bold), Cys to
Ser substitution (#), YTE triple mutation (bold and underlined), allotype G1m(f)(bold italics)
MGWSCIILFLVATATGVHSNVNHKPSNTKVDKKVEPKSS(#)DKTHTCPPCPAPELLGGPSVFLFPPKPKD
TLYITREPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSR [symbol] E [symbol] TKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 54: mature human IgG1 Fc with mouse heavy chain MIgG Vh signal sequence (bold), N-
terminal ISAMVRS amino acid residues added (italicized), M428L, N434S mutations (bold/underlined),
G4S linker (italicized), and C-terminal C-myc-tag (underlined), allotype G1m(f)(bold italics)
MGWSCIILFLVATATGVHSISAMVRSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPSR [symbol] E [symbol] TKNQVSLTCLVKGFYPSDIAVEWESNGQPENNYKTTPPVLDSDGS
FFLYSKLTVDKSRWQQGNVFSCSVLHEALHSHYTQKSLSLSPGGGGGSEQKLISEEDL
SEQ ID NO: 55: mature human IgG1 Fc with mouse heavy chain MIgG Vh signal sequence (bold), N-
terminal ISAMVRS amino acid residues added (italicized), M428L, N434S mutations (bold/underlined),
G4S linker (italicized), C-terminal C-myc-tag (underlined), allotype G1m(fa)(bold italics)
MGWSCIILFLVATATGVHSISAMVRSDKTHTCPPCPAPELLGGPSVFLFPPKPKDTLMISRTPEVTCVVVD
VSHED PEVKFNWYVDGVEVHNAKTKPREEQYNSTYRVVSVLTVLHQDWLNGKEYKCKVSNKALPAPIEK
TISKAKGQPREPQVYTLPPSR [symbol] E [symbol] TKNQVSLTCLVKGFYPSDIAVEWESNGQPENNYKTTPPVLDSDGSF
FLYSKLTVDKSRWQQGNVFSCSVLHEALHSHYTQKSLSLSPGGGGGSEQKLISEEDL
SEQ ID NO: 56: mature human IgG1 Fc with mouse heavy chain MIgG Vh signal sequence (bold), N-
terminal ISAMVRS amino acid residues added (italicized), YTE triple mutant (bold/underlined), G4S linker
(italicized), and C-terminal C-myc-tag (underlined), allotype G1m(f)(bold italics)
MGWSCIILFLVATATGVHSISAMVRSDKTHTCPPCPAPELLGGPSVFLFPPKPKDTLYITREPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPSR [symbol] E [symbol] TKNQVSLTCLVKGFYPSDIAVEWESNGQPENNYKTTPPVLDSDGS
FFLYSKLTVDKSRWQQGNVFSCSVMHEALHNHYTQKSLSLSPGGGGGSEQKLISEEDL
SEQ ID NO: 57: mature human IgG1 Fc with mouse heavy chain MIgG Vh signal sequence (bold), N-
terminal ISAMVRS amino acid residues added (italicized), YTE triple mutant (bold/underlined), G4S linker
(italicized), C-terminal C-myc-tag (underlined), allotype G1m(fa)(bold italics)
MGWSCIILFLVATATGVHSISAMVRSDKTHTCPPCPAPELLGGPSVFLFPPKPKDTLYITREPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPSR [symbol] E [symbol] TKNQVSLTCLVKGFYPSDIAVEWESNGQPENNYKTTPPVLDSDGSF
FLYSKLTVDKSRWQQGNVFSCSVMHEALHNHYTQKSLSLSPGGGGGSEQKLISEEDL
SEQ ID NO: 58: mature human IgG1 with mouse heavy chain MIgG1 signal sequence (bold), Cys to Ser
substitution (#), C-terminal G4S (italics), and C-terminal IgA peptide (underline), allotype G1m(fa)(bold
italics)
MGWSCIILFLVATATGVHSEPKSS(#)DKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPSR [symbol] E [symbol] TKNQVSLTCLVKGFYPSDIAVEWESNGQPENNYKTTPPVLDSDGSF
FLYSKLTVDKSRWQQGNVFSCSVMHEALHNHYTQKSLSLSPGGGGGSQRNPRLRLIRRHPTLRIPPI
SEQ ID NO: 59: mature human IgG1 with mouse heavy chain MIgG1 signal sequence (bold), Cys to Ser
substitution (#), M428L, N434S mutations (bold/underlined), C-terminal G4S (italics), and C-terminal IgA
peptide (underline), allotype G1m(fa)(bold italics)
MGWSCIILFLVATATGVHSEPKSS(#)DKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPSR [symbol] E [symbol] TKNQVSLTCLVKGFYPSDIAVEWESNGQPENNYKTTPPVLDSDGSF
FLYSKLTVDKSRWQQGNVFSCSVLHEALHSHYTQKSLSLSPGGGGGSQRNPRLRLIRRHPTLRIPPI
SEQ ID NO: 60: mature human Fc IgG1, Z₁ is Cys or Ser, and wherein X₁ is Met or Trp, X₂ is Ser or Thr,
X₃ is Thr or Glu, X₄ is Asp or Glu, and X₅ is Leu or Met, X₆ is Met or Leu, and X₇ is Asn or Ser
NVNHKPSNTKVDKKVEPKS [symbol] DKTHTCPPCPAPELLGGPSVFLFPPKPKDTL [symbol] R [symbol] PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFF
LYSKLTVDKSRWQQGNVFSCSV [symbol] HEALH [symbol] HYTQKSLSLSPGK
SEQ ID NO: 61: mature human Fc IgG1, Cys to Ser substitution (#), and wherein X₁ is Met or Trp, X₂ is
Ser or Thr, X₃ is Thr or Glu, X4 is Asp or Glu, and X₅ is Leu or Met, X₆ is Met or Leu, and X₇ is Asn or
Ser
NVNHKPSNTKVDKKVEPKSS(#)DKTHTCPPCPAPELLGGPSVFLFPPKPKDTL [symbol] R [symbol] PEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSR [symbol] E [symbol] TKNQVSLTCLVKGFYPSDIAVEWESNGQPENNYKTTPPVLDSDGSF
FLYSKLTVDKSRWQQGNVFSCSV [symbol] HEALH [symbol] HYTQKSLSLSPGK
SEQ ID NO: 62: mature human IgG1 Fc, Cys to Ser substitution (#), X₄ is Asp or Glu, and X₅ is Leu or
Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 63: mature human IgG1 Fc, Cys to Ser substitution (#), allotype G1m(f)(bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 64: mature human IgG1 Fc, Cys to Ser substitution (#), allotype G1m(fa)(bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 65: mature human IgG1 Fc, Cys to Ser substitution (#), M428L, N434S mutations
(Bold/Underlined), allotype G1m(fa)(bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVLHEALHSHYTQKSLSLSPGK
SEQ ID NO: 66: mature human IgG1 Fc, Cys to Ser substitution (#), M428L, N434S mutations
(Bold/Underlined), allotype G1m(f)(bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVLHEALHSHYTQKSLSLSPGK
SEQ ID NO: 67: mature human IgG1 Fc, Cys to Ser substitution (#), YTE triple mutation (bold and
underlined), allotype G1m(fa)(bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 68: mature human IgG1 Fc, Cys to Ser substitution (#), YTE triple mutation (bold and
underlined), allotype G1m(f)(bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 69: mature human Fc IgG1, Z₁ is Cys or Ser, and wherein X₁ is Met or Trp, X₂ is Ser or Thr,
X₃ is Thr or Glu, X₄ is Asp or Glu, and X₅ is Leu or Met, X₆ is Met or Leu, and X₇ is Asn or Ser
NVNHKPSNTKVDKKVEPKS [symbol] DKTHTCPPCPAPELLGGPSVFLFPPKPKDTL [symbol] R [symbol] PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFF
LYSKLTVDKSRWQQGNVFSCSV [symbol] HEALH [symbol] HYTQKSLSLSPG
SEQ ID NO: 70: mature human Fc IgG1, Cys to Ser substitution (#), and wherein X₁ is Met or Trp, X₂ is
Ser or Thr, X₃ is Thr or Glu, X₄ is Asp or Glu, and X₅ is Leu or Met, X₆ is Met or Leu, and X₇ is Asn or
Ser
NVNHKPSNTKVDKKVEPKSS(#)DKTHTCPPCPAPELLGGPSVFLFPPKPKDTL [symbol] R [symbol] PEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSR [symbol] E [symbol] TKNQVSLTCLVKGFYPSDIAVEWESNGQPENNYKTTPPVLDSDGSF
FLYSKLTVDKSRWQQGNVFSCSV [symbol] HEALH [symbol] HYTQKSLSLSPG
SEQ ID NO: 71: mature human IgG1 Fc, Cys to Ser substitution (#), X₄ is Asp or Glu, and X₅ is Leu or
Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 72: mature human IgG1 Fc, Cys to Ser substitution (#), allotype G1m(f)(bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 73: mature human IgG1 Fc, Cys to Ser substitution (#), allotype G1m(fa)(bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 74: mature human IgG1 Fc, Cys to Ser substitution (#), M428L, N434S mutations
(Bold/Underlined), allotype G1m(fa)(bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVLHEALHSHYTQKSLSLSPG
SEQ ID NO: 75: mature human IgG1 Fc, Cys to Ser substitution (#), M428L, N434S mutations
(Bold/Underlined), allotype G1m(f)(bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVLHEALHSHYTQKSLSLSPG
SEQ ID NO: 76: mature human IgG1 Fc, Cys to Ser substitution (#), YTE triple mutation (bold and
underlined), allotype G1m(fa)(bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 77: mature human IgG1 Fc, Cys to Ser substitution (#), YTE triple mutation (bold and
underlined), allotype G1m(f)(bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 78: mature human Fc IgG1, Z₁ is Cys or Ser, and wherein X₁ is Met or Trp, X₂ is Ser or Thr,
X₃ is Thr or Glu, X₄ is Asp or Glu, and X₅ is Leu or Met, X₆ is Met or Leu, and X₇ is Asn or Ser
VNHKPSNTKVDKKVEPKS [symbol] DKTHTCPPCPAPELLGGPSVFLFPPKPKDTL [symbol] R [symbol] PEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSV [symbol] HEALH [symbol] HYTQKSLSLSPGK
SEQ ID NO: 79: mature human Fc IgG1, Cys to Ser substitution (#), and wherein X₁ is Met or Trp, X₂ is
Ser or Thr, X₃ is Thr or Glu, X₄ is Asp or Glu, and X₅ is Leu or Met, X₆ is Met or Leu, and X₇ is Asn or
Ser
VNHKPSNTKVDKKVEPKSS(#)DKTHTCPPCPAPELLGGPSVFLFPPKPKDTL [symbol] R [symbol] PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFF
LYSKLTVDKSRWQQGNVFSCSV [symbol] HEALH [symbol] HYTQKSLSLSPGK
SEQ ID NO: 80: mature human IgG1 Fc, Cys to Ser substitution (#), X₄ is Asp or Glu, and X₅ is Leu or
Met
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 81: mature human IgG1 Fc, Cys to Ser substitution (#), allotype G1m(f)(bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 82: mature human IgG1 Fc, Cys to Ser substitution (#), allotype G1m(fa)(bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 83: mature human IgG1 Fc, Cys to Ser substitution (#), M428L, N434S mutations
(Bold/Underlined), allotype G1m(fa)(bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVLHEALHSHYTQKSLSLSPGK
SEQ ID NO: 84: mature human IgG1 Fc, Cys to Ser substitution (#), M428L, N434S mutations
(Bold/Underlined), allotype G1m(f)(bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVLHEALHSHYTQKSLSLSPGK
SEQ ID NO: 85: mature human IgG1 Fc, Cys to Ser substitution (#), YTE triple mutation (bold and
underlined), allotype G1m(fa)(bold italics)
VNHKPSNTKVDKKVEPKSS(#)DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 86: mature human IgG1 Fc, Cys to Ser substitution (#), YTE triple mutation (bold and
underlined), allotype G1m(f)(bold italics)
VNHKPSNTKVDKKVEPKSS(#)DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 87: mature human Fc IgG1, Z₁ is Cys or Ser, and wherein X₁ is Met or Trp, X₂ is Ser or Thr,
X₃ is Thr or Glu, X₄ is Asp or Glu, and X₅ is Leu or Met, X₆ is Met or Leu, and X₇ is Asn or Ser
VNHKPSNTKVDKKVEPKS [symbol] DKTHTCPPCPAPELLGGPSVFLFPPKPKDTL [symbol] R [symbol] PEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSV [symbol] HEALH [symbol] HYTQKSLSLSPG
SEQ ID NO: 88: mature human Fc IgG1, Cys to Ser substitution (#), and wherein X₁ is Met or Trp, X₂ is
Ser or Thr, X₃ is Thr or Glu, X₄ is Asp or Glu, and X₅ is Leu or Met, X₆ is Met or Leu, and X₇ is Asn or
Ser
VNHKPSNTKVDKKVEPKSS(#)DKTHTCPPCPAPELLGGPSVFLFPPKPKDTL [symbol] R [symbol] PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFF
LYSKLTVDKSRWQQGNVFSCSV [symbol] HEALH [symbol] HYTQKSLSLSPG
SEQ ID NO: 89: mature human IgG1 Fc, Cys to Ser substitution (#), X₄ is Asp or Glu, and X₅ is Leu or
Met
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 90: mature human IgG1 Fc, Cys to Ser substitution (#), allotype G1m(f)(bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 91: mature human IgG1 Fc, Cys to Ser substitution (#), allotype G1m(fa)(bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 92: mature human IgG1 Fc, Cys to Ser substitution (#), M428L, N434S mutations
(Bold/Underlined), allotype G1m(fa)(bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVLHEALHSHYTQKSLSLSPG
SEQ ID NO: 93: mature human IgG1 Fc, Cys to Ser substitution (#), M428L, N434S mutations
(Bold/Underlined), allotype G1m(f)(bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVLHEALHSHYTQKSLSLSPG
SEQ ID NO: 94: mature human IgG1 Fc, Cys to Ser substitution (#), YTE triple mutation (bold and
underlined), allotype G1m(fa)(bold italics)
VNHKPSNTKVDKKVEPKSS(#)DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 95: mature human IgG1 Fc, Cys to Ser substitution (#), YTE triple mutation (bold and
underlined), allotype G1m(f)(bold italics)
VNHKPSNTKVDKKVEPKSS(#)DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSR [symbol] E [symbol] TKNQVSLTCLVKGFYPSDIAVEWESNGQPENNYKTTPPVLDSDGSFFLY
SKLTVDKSRWQQGNVFSCSVMHEALHNHYTQKSLSLSPG

As defined herein, an Fc domain includes two Fc domain monomers that are dimerized by the interaction between the C_{H}3 antibody constant domains, as well as one or more disulfide bonds that form between the hinge domains of the two dimerizing Fc domain monomers. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present invention binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

In some embodiments, the Fc domain monomer or Fc domain of the invention is an aglycosylated Fc domain monomer or Fc domain (e.g., an Fc domain monomer or and Fc domain that maintains engagement to an Fc receptor (e.g., FcRn). For example, the Fc domain is an aglycosylated IgG1 variants that maintains engagement to an Fc receptor (e.g., an IgG1 having an amino acid substitution at N297 and/or T299 of the glycosylation motif). Exemplary aglycosylated Fc domains and methods for making aglycosylated Fc domains are known in the art, for example, as described in Sazinsky S. L. et al., Aglycosylated immunoglobulin G1 variants productively engage activating Fc receptors, PNAS, 2008, 105(51):20167-20172, which is incorporated herein in its entirety.

In some embodiments, the Fc domain or Fc domain monomer of the invention is engineered to enhance binding to the neonatal Fc receptor (FcRn). For example, the Fc domain may include the triple mutation corresponding to M252Y/S254T/T256E (YTE) (e.g., an IgG1, such as a human or humanized IgG1 having a YTE mutation, for example SEQ ID NO: 33, SEQ ID NO: 36, SEQ ID NO: 38, SEQ ID NO: 39, SEQ ID NO: 41, SEQ ID NO: 42, SEQ ID NO: 43, SEQ ID NO: 52, SEQ ID NO: 53, SEQ ID NO: 56, or SEQ ID NO: 57). The Fc domain may include the double mutant corresponding to M428L/N434S (LS) (e.g., an IgG1, such as a human or humanized IgG1 having an LS mutation, such as SEQ ID NO: 37, SEQ ID NO: 39, SEQ ID NO: 44, SEQ ID NO: 45, SEQ ID NO: 46, SEQ ID NO: 50, SEQ ID NO: 51, SEQ ID NO: 54, SEQ ID NO: 55, or SEQ ID NO: 59). The Fc domain may include the single mutant corresponding to N434H (e.g., an IgG1, such as a human or humanized IgG1 having an N434H mutation). The Fc domain may include the single mutant corresponding to C220S (e.g., and IgG1, such as a human or humanized IgG1 having a C220S mutation, such as SEQ ID NO: 34, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68). The Fc domain may include a combination of one or more of the above-described mutations that enhance binding to the FcRn. Enhanced binding to the FcRn may increase the half-life Fc domain-containing conjugate. For example, incorporation of one or more amino acid mutations that increase binding to the FcRn (e.g., a YTE mutation, an LS mutation, or an N434H mutation) may increase the half-life of the conjugate by 5%, 10%, 15%, 20%, 30%, 40%, 50%, 60%, 70%, 80%, 90%, 100%, 200%, 300%, 400%, 500% or more relative to a conjugate having an the corresponding Fc domain without the mutation that enhances FcRn binding. Exemplary Fc domains with enhanced binding to the FcRN and methods for making Fc domains having enhanced binding to the FcRN are known in the art, for example, as described in Maeda, A. et al., Identification of human IgG1 variant with enhanced FcRn binding and without increased binding to rheumatoid factor autoantibody, MABS, 2017, 9(5):844-853, which is incorporated herein in its entirety. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-95 may be mutated to include a YTE mutation, an LS mutation, and/or an N434H mutation by mutating the “corresponding residues” of the amino acid sequence.

As used herein, a sulfur atom “corresponding to” a particular cysteine residue of a particular SEQ ID NO. should be understood to include the sulfur atom of any cysteine residue that one of skill in the art would understand to align to the particular cysteine of the particular sequence. The protein sequence alignment of human IgG1 (UniProtKB: P01857; SEQ ID NO: 121), human IgG2 (UniProtKB: P01859; SEQ ID NO: 122), human IgG3 (UniProtKB: P01860; SEQ ID NO: 123), and human IgG4 (UniProtKB: P01861; SEQ ID NO: 124) is provided below (aligned with Clustal Omega Multiple Pairwise Alignment). The alignment indicates cysteine residues (e.g., sulfur atoms of cysteine residues) that “correspond to” one another (in boxes and indicated by the symbol). One of skill in the art would readily be able to perform such an alignment with any IgG variant of the invention to determine the sulfur atom of a cysteine that corresponds to any sulfur atom of a particular cysteine of a particular SEQ ID NO. described herein (e.g., any one of SEQ ID NOs: 1-95). For example, one of skill in the art would readily be able to determine that Cys10 of SEQ ID NO: 10 (the first cysteine of the conserved CPPC motif of the hinge region of the Fc domain) corresponds to, for example, Cys109 of IgG1, Cys106 of IgG2, Cys156 of IgG3, Cys29 of SEQ ID NO: 1, Cys9 of SEQ ID NO: 2, Cys30 of SEQ ID NO: 3, or Cys10 of SEQ ID NO: 10.

In some embodiments, the Fc domain or Fc domain monomer of the invention has the sequence of any one of SEQ ID NOs: 39-95 may further include additional amino acids at the N-terminus (Xaa)x and/or additional amino acids at the C-terminus (Xaa)z, wherein Xaa is any amino acid and x and z are a whole number greater than or equal to zero, generally less than 100, preferably less than 10 and more preferably 0, 1, 2, 3, 4, or 5. In some embodiments, the additional amino acids are least 70% (e.g., 70%, 75%, 80%, 85%, 90%, 95%, 96%, 97%, 98%, 99%, or 100%) identical to one or more consecutive amino acids of SEQ ID NO: 94. For example, the additional amino acids may be a single amino acid on the C-terminus corresponding to Lys330 of IgG1 (SEQ ID NO: 121).

As used herein, a nitrogen atom “corresponding to” a particular lysine residue of a particular SEQ ID NO. should be understood to include the nitrogen atom of any lysine residue that one of skill in the art would understand to align to the particular lysine of the particular sequence. The protein sequence alignment of human IgG1 (UniProtKB: P01857; SEQ ID NO: 121), human IgG2 (UniProtKB: P01859; SEQ ID NO: 122), human IgG3 (UniProtKB: P01860; SEQ ID NO: 123), and human IgG4 (UniProtKB: P01861; SEQ ID NO: 124) is provided below (aligned with Clustal Omega Multiple Pairwise Alignment). The alignment indicates lysine residues (e.g., nitrogen atoms of lysine residues) that “correspond to” one another (in boxes and indicated by the * symbol). One of skill in the art would readily be able to perform such an alignment with any IgG variant of the invention to determine the nitrogen atom of a lysine that corresponds to any nitrogen atom of a particular lysine of a particular SEQ ID NO. described herein (e.g., any one of SEQ ID NOs: 1-95). For example, one of skill in the art would readily be able to determine that Lys35 of SEQ ID NO: 10 corresponds to, for example, Lys129 of IgG1, Lys126 of IgG2, Lys176 of IgG3, Lys51 of SEQ ID NO: 1, Lys31 of SEQ ID NO: 2, Lys50 of SEQ ID NO: 3, or Lys30 of SEQ ID NO: 10.

Protein sequence alignment of IgG1 (SEQ ID NO: 121), IgG2 (SEQ ID NO: 122),
IgG3 (SEQ ID NO: 123), and IgG4 (SEQ ID NO: 124)

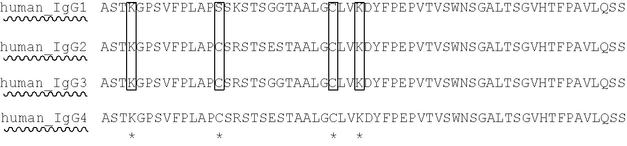

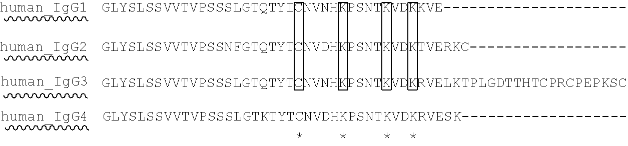

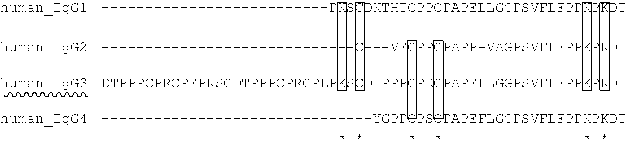

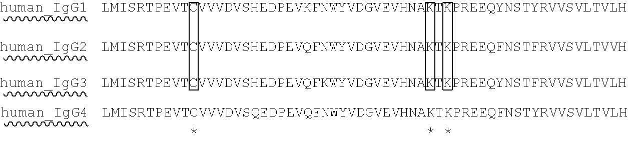

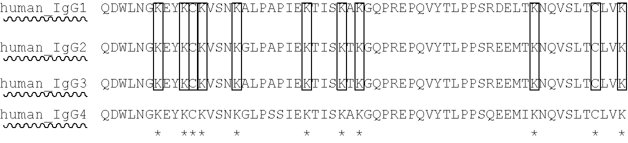

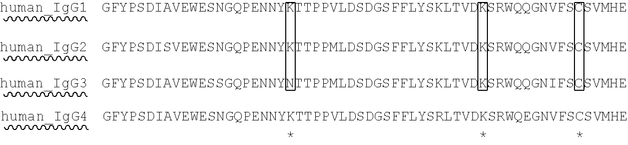

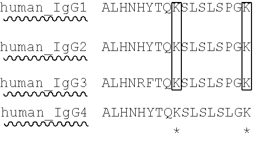

In some embodiments, the Fc domain monomer includes less than about 300 amino acid residues (e.g., less than about 300, less than about 295, less than about 290, less than about 285, less than about 280, less than about 275, less than about 270, less than about 265, less than about 260, less than about 255, less than about 250, less than about 245, less than about 240, less than about 235, less than about 230, less than about 225, or less than about 220 amino acid residues). In some embodiments, the Fc domain monomer is less than about 40 kDa (e.g., less than about 35 kDa, less than about 30 kDa, less than about 25 kDa).

In some embodiments, the Fc domain monomer includes at least 200 amino acid residues (e.g., at least 210, at least 220, at least 230, at least 240, at least 250, at least 260, at least 270, at least 280, at least 290, or at least 300 amino residues). In some embodiments, the Fc domain monomer is at least 20 kDa (e.g., at least 25 kDa, at least 30 kDa, or at least 35 kDa).

In some embodiments, the Fc domain monomer includes 200 to 400 amino acid residues (e.g., 200 to 250, 250 to 300, 300 to 350, 350 to 400, 200 to 300, 250 to 350, or 300 to 400 amino acid residues). In some embodiments, the Fc domain monomer is 20 to 40 kDa (e.g., 20 to 25 kDa, 25 to 30 kDa, 35 to 40 kDa, 20 to 30 kDa, 25 to 35 kDa, or 30 to 40 KDa).

In some embodiments, the Fc domain monomer includes an amino acid sequence at least 90% identical (e.g., at least 95%, at least 98%) to the sequence of any one of SEQ ID NOs: 1-95, or a region thereof. In some embodiments, the Fc domain monomer includes the amino acid sequence of any one of SEQ ID NOs: 1-95, or a region thereof.

In some embodiments, the Fc domain monomer includes a region of any one of SEQ ID NOs: 1-95, wherein the region includes positions 220, 252, 254, and 256. In some embodiments, the region includes at least 40 amino acid residues, at least 50 amino acid residues, at least 60 amino acid residues, at least 70 amino acids residues, at least 80 amino acids residues, at least 90 amino acid residues, at least 100 amino acid residues, at least 110 amino acid residues, at least 120 amino residues, at least 130 amino acid residues, at least 140 amino acid residues, at least 150 amino acid residues, at least 160 amino acid residues, at least 170 amino acid residues, at least 180 amino acid residues, at least 190 amino acid residues, or at least 200 amino acid residues.

### Activation of Immune Cells

Fc-gamma receptors (FcγRs) bind the Fc portion of immunoglobulin G (IgG) and play important roles in immune activation and regulation. For example, the IgG Fc domains in immune complexes (ICs) engage FcγRs with high avidity, thus triggering signaling cascades that regulate immune cell activation. The human FcγR family contains several activating receptors (FcγRI, FcγRIIa, FcγRIIc, FcγRIIIa, and FcγRIIIb) and one inhibitory receptor (FcγRIIb). FcγR signaling is mediated by intracellular domains that contain immune tyrosine activating motifs (ITAMs) for activating FcγRs and immune tyrosine inhibitory motifs (ITIM) for inhibitory receptor FcγRIIb. In some embodiments, FcγR binding by Fc domains results in ITAM phosphorylation by Src family kinases; this activates Syk family kinases and induces downstream signaling networks, which include PI3K and Ras pathways.

In the conjugates described herein, the portion of the conjugates including monomers or dimers of RSV F protein inhibitors bind to and inhibits viral RSV F protein leading to inhibition of viral replication, while the Fc domain portion of the conjugates bind to FcγRs (e.g., FcRn, FcγRI, FcγRIIa, FcγRIIc, FcγRIIIa, and FcγRIIIb) on immune cells and activate phagocytosis and effector functions, such as antibody-dependent cell-mediated cytotoxicity (ADCC), thus leading to the engulfment and destruction of viral particles by immune cells and further enhancing the antiviral activity of the conjugates. Examples of immune cells that may be activated by the conjugates described herein include, but are not limited to, macrophages, neutrophils, eosinophils, basophils, lymphocytes, follicular dendritic cells, natural killer cells, and mast cells.

### Tissue Distribution

After a therapeutic enters the systemic circulation, it is distributed to the body's tissues. Distribution is generally uneven because of different in blood perfusion, tissue binding, regional pH, and permeability of cell membranes. The entry rate of a drug into a tissue depends on the rate of blood flow to the tissue, tissue mass, and partition characteristics between blood and tissue. Distribution equilibrium (when the entry and exit rates are the same) between blood and tissue is reached more rapidly in richly vascularized areas, unless diffusion across cell membranes is the rate-limiting step. The size, shape, charge, target binding, FcRn and target binding mechanisms, route of administration, and formulation affect tissue distribution.

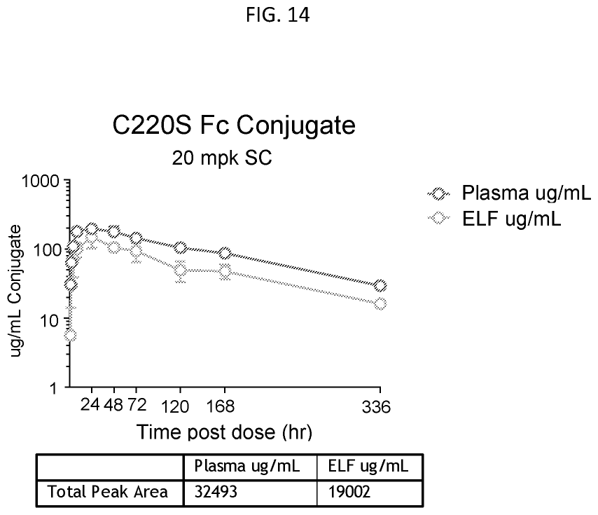

In some instances, the conjugates described herein may be optimized to distribute to lung tissue. In some instances, the conjugates have a concentration ratio of distribution in epithelial lining fluid of at least 30% the concentration of the conjugate in plasma within 2 hours after administration. In certain embodiments, ratio of the concentration is at least 45% within 2 hours after administration. In some embodiments, the ratio of concentration is at least 55% within 2 hours after administration. In particular, the ratio of concentration is at least 60% within 2 hours after administration. As shown in Example 33 and FIG. 14, by 2 hours post injection, a conjugate having an Fc domain (SEQ ID NO: 64) decorated with one or more small molecule antiviral inhibitors ELF levels are surprisingly ˜60% of plasma exposure levels as measured by AUC across the rest of the time course indicating nearly immediate partitioning of the conjugate from plasma to the ELF in the lung. This demonstrates that an Fc containing conjugate rapidly distributes to lung, and maintains high concentrations in lung relative to levels in plasma.

### IV. Albumin Proteins and Albumin Protein-Binding Peptides

### Albumin Proteins

An albumin protein of the invention may be a naturally-occurring albumin or a variant thereof, such as an engineered variant of a naturally-occurring albumin protein. Variants include polymorphisms, fragments such as domains and sub-domains, and fusion proteins. An albumin protein may include the sequence of an albumin protein obtained from any source. Preferably the source is mammalian, such as human or bovine. Most preferably, the albumin protein is human serum albumin (HSA), or a variant thereof. Human serum albumins includes any albumin protein having an amino acid sequence naturally occurring in humans, and variants thereof. An albumin protein coding sequence is obtainable by methods know to those of skill in the art for isolating and sequencing cDNA corresponding to human genes. An albumin protein of the invention may include the amino acid sequence of human serum albumin (HSA), provided in SEQ ID NO: 96 or SEQ ID NO: 97, or the amino acid sequence of mouse serum albumin (MSA), provided in SEQ ID NO: 98, or a variant or fragment thereof, preferably a functional variant or fragment thereof. A fragment or variant may or may not be functional, or may retain the function of albumin to some degree. For example, a fragment or variant may retain the ability to bind to an albumin receptor, such as HSA or MSA, by at least 10%, 20%, 30%, 40%, 50%, 60%, 70%, 80%, 90%, 100%, or 105% of the ability of the parent albumin (e.g., the parent albumin from which the fragment or variant is derived). Relative binding ability may be determined by methods known in the art, such as by surface plasmon resonance.

The albumin protein may be a naturally-occurring polymorphic variant of an albumin protein, such as human serum albumin. Generally, variants or fragments of human serum albumin will have at least 5%, 10%, 15%, 20%, 30%, 40%, 50%, 60%, or 70%, and preferably 80%, 90%, 95%, 100%, or 105% or more of human serum albumin or mouse serum albumin's ligand binding activity.

The albumin protein may include the amino acid sequence of bovine serum albumin. Bovine serum albumin proteins include any albumin having an amino acid sequence naturally occurring in cows, for example, as described by Swissprot accession number P02769, and variants thereof as defined herein. Bovine serum albumin proteins also includes fragments of full-length bovine serum albumin or variants thereof, as defined herein.

The albumin protein may include the sequence of an albumin derived from one of serum albumin from dog (e.g., Swissprot accession number P49822-1), pig (e.g., Swissprot accession number P08835-1), goat (e.g., Sigma product no. A2514 or A4164), cat (e.g., Swissprot accession number P49064-1), chicken (e.g., Swissprot accession number P19121-1), ovalbumin (e.g., chicken ovalbumin) (e.g., Swissprot accession number P01012-1), turkey ovalbumin (e.g., Swissprot accession number O73860-1), donkey (e.g., Swissprot accession number Q5XLE4-1), guinea pig (e.g., Swissprot accession number Q6WDN9-1), hamster (e.g., as described in DeMarco et al. International Journal for Parasitology 37(11): 1201-1208 (2007)), horse (e.g., Swissprot accession number P35747-1), rhesus monkey (e.g., Swissprot accession number Q28522-1), mouse (e.g., Swissprot accession number P07724-1), pigeon (e.g., as defined by Khan et al. Int. J. Biol. Macromol. 30(3-4), 171-8 (2002)), rabbit (e.g., Swissprot accession number P49065-1), rat (e.g., Swissprot accession number P02770-1) or sheep (e.g., Swissprot accession number P14639-1), and includes variants and fragments thereof as defined herein.

Many naturally-occurring mutant forms of albumin are known to those skilled in the art. Naturally-occurring mutant forms of albumin are described in, for example, Peters, et al. All About Albumin: Biochemistry, Genetics and Medical Applications, Academic Press, Inc., San Diego, Calif., p. 170-181 (1996).

Albumin proteins of the invention include variants of naturally-occurring albumin proteins. A variant albumin refers to an albumin protein having at least one amino acid mutation, such as an amino acid mutation generated by an insertion, deletion, or substitution, either conservative or non-conservative, provided that such changes result in an albumin protein for which at least one basic property has not been significantly altered (e.g., has not been altered by more than 5%, 10%, 15%, 20%, 25%, 30%, 35%, or 40%). Exemplary properties which may define the activity of an albumin protein include binding activity (e.g., including binding specificity or affinity to bilirubin, or a fatty acid such as a long-chain fatty acid), osmolarity, or behavior in a certain pH-range.

Typically an albumin protein variant will have at least 40%, at least 50%, at least 60%, and preferably at least 70%, at least 80%, at least 90%, at least 95%, at least 96%, at least 97%, at least 98%, or at least 99% amino acid sequence identity with a naturally-occurring albumin protein, such as the albumin protein of any one of SEQ ID NOs: 96-98.

Methods for the production and purification of recombinant human albumins are well-established (Sleep et al. Biotechnology, 8(1):42-6 (1990)), and include the production of recombinant human albumin for pharmaceutical applications (Bosse et al. J Clin Pharmacol 45(1):57-67 (2005)). The three-dimensional structure of HSA has been elucidated by X-ray crystallography (Carter et al. Science. 244(4909): 1195-8(1998)); Sugio et al. Protein Eng. 12(6):439-46 (1999)). The HSA polypeptide chain has 35 cysteine residues, which form 17 disulfide bonds, and one unpaired (e.g., free) cysteine at position 34 of the mature protein. Cys-34 of HSA has been used for conjugation of molecules to albumin (Leger et al. Bioorg Med Chem Lett 14(17):4395-8 (2004); Thibaudeau et al. Bioconjug Chem 16(4):1000-8 (2005)), and provides a site for site-specific conjugation.

SEQ ID NO: 96
(Human serum albumin (HSA), variant 1)
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL
SEQ ID NO: 97
(Human serum albumin (HSA), variant 2)
RGVFRRDAHKSEVAHRFKDLGEENFKALVLIAFAQYLQQCPFEDHVKLVN
EVTEFAKTCVADESAENCDKSLHTLFGDKLCTVATLRETYGEMADCCAKQ
EPERNECFLQHKDDNPNLPRLVRPEVDVMCTAFHDNEETFLKKYLYEIAR
RHPYFYAPELLFFAKRYKAAFTECCQAADKAACLLPKLDELRDEGKASSA
KQRLKCASLQKFGERAFKAWAVARLSQRFPKAEFAEVSKLVTDLTKVHTE
CCHGDLLECADDRADLAKYICENQDSISSKLKECCEKPLLEKSHCIAEVE
NDEMPADLPSLAADFVESKDVCKNYAEAKDVFLGMFLYEYARRHPDYSVV
LLLRLAKTYETTLEKCCAAADPHECYAKVFDEFKPLVEEPQNLIKQNCEL
FEQLGEYKFQNALLVRYTKKVPQVSTPTLVEVSRNLGKVGSKCCKHPEAK
RMPCAEDYLSVVLNQLCVLHEKTPVSDRVTKCCTESLVNRRPCFSALEVD
ETYVPKEFNAETFTFHADICTLSEKERQIKKQTALVELVKHKPKATKEQL
KAVMDDFAAFVEKCCKADDKETCFAEEGKKLVAASQAALGL
SEQ ID NO: 98
(Mouse serum albumin (MSA))
RGVFRREAHKSEIAHRYNDLGEQHFKGLVLIAFSQYLQKCSYDEHAKLVQ
EVTDFAKTCVADESAANCDKSLHTLFGDKLCAIPNLRENYGELADCCTKQ
EPERNECFLQHKDDNPSLPPFERPEAEAMCTSFKENPTTFMGHYLHEVAR
RHPYFYAPELLYYAEQYNEILTQCCAEADKESCLTPKLDGVKEKALVSSV
RQRMKCSSMQKFGERAFKAWAVARLSQTFPNADFAEITKLATDLTKVNKE
CCHGDLLECADDRAELAKYMCENQATISSKLQTCCDKPLLKKAHCLSEVE
HDTMPADLPAIAADFVEDQEVCKNYAEAKDVFLGTFLYEYSRRHPDYSVS
LLLRLAKKYEATLEKCCAEANPPACYGTVLAEFQPLVEEPKNLVKTNCDL
YEKLGEYGFQNAILVRYTQKAPQVSTPTLVEAARNLGRVGTKCCTLPEDQ
RLPCVEDYLSAILNRVCLLHEKTPVSEHVTKCCSGSLVERRPCFSALTVD
ETYVPKEFKAETFTFHSDICTLPEKEKQIKKQTALAELVKHKPKATAEQL
KTVMDDFAQFLDTCCKAADKDTCFSTEGPNLVTRCKDALA

### Conjugation of Albumin Proteins

An albumin protein of the invention may be conjugated to (e.g., by way of a covalent bond) to any compound of the invention (e.g., by way of the linker portion of a RSV F protein inhibitor monomer or dimer). The albumin protein may be conjugated to any compound of the invention by any method well-known to those of skill in the art for producing small-molecule-protein conjugates. This may include covalent conjugation to a solvent-exposed amino acid, such as a solvent exposed cysteine or lysine. For example, human serum albumin may be conjugated to a compound of the invention by covalent linkage to the sulfur atom corresponding to Cys34 of SEQ ID NO: 96 or Cys40 of SEQ ID NO: 97.

An albumin protein of the invention may be conjugated to any compound of the invention by way of an amino acid located within 10 amino acid residues of the C-terminal or N-terminal end of the albumin protein. An albumin protein may include a C-terminal or N-terminal polypeptide fusion of 1, 2, 3, 4, 5, 6, 7, 8, 9, 10, 15, or 20 or more amino acid. The C-terminal or N-terminal polypeptide fusion may include one or more solvent-exposed cysteine or lysine residues, which may be used for covalent conjugation of a compound of the invention (e.g., conjugation to a RSV F protein inhibitor monomer or dimer, including by way of a linker).

Albumin proteins of the invention include any albumin protein which has been engineered to include one or more solvent-exposed cysteine or lysine residues, which may provide a site for conjugation to a compound of the invention (e.g., conjugation to a RSV F protein inhibitor monomer or dimer, including by way of a linker). Most preferably, the albumin protein will contain a single solvent-exposed cysteine or lysine, thus enabling site-specific conjugation of a compound of the invention.

Exemplary methods for the production of engineered variants of albumin proteins that include one or more conjugation-competent cysteine residues are provided in U.S. Patent Application No. 2017/0081389, which is incorporated herein by reference in its entirety. Briefly, preferred albumin protein variants are those including a single, solvent-exposed, unpaired (e.g., free) cysteine residue, thus enabling site-specific conjugation of a linker to the cysteine residue.

Albumin proteins which have been engineered to enable chemical conjugation to a solvent-exposed, unpaired cysteine residue include the following albumin protein variants:
(a) an albumin protein having a substitution of a non-cysteine amino acid residue with a cysteine at an amino acid residue corresponding to any of L585, D1, A2, D562, A364, A504, E505, T79, E86, D129, D549, A581, D121, E82, S270, Q397, and A578 of SEQ ID NO: 96;
(b) an albumin protein having an insertion of a cysteine at a position adjacent the N- or C-terminal side of an amino acid residue corresponding to any of L585, D1, A2, D562, A364, A504, E505, T79, E86, D129, D549, A581, D121, E82, S270, Q397, and A578 of SEQ ID NO: 96;
(c) an albumin protein engineered to have an unpaired cysteine having a free thiol group at a residue corresponding to any of C369, C361, C91, C177, C567, C316, C75, C169, C124, or C558 of SEQ ID NO: 96, and which may or may not be generated by deletion or substitution of a residue corresponding to C360, C316, C75, C168, C558, C361, C91, C124, C169, or C567 of SEQ ID NO: 96; and/or
(d) addition of a cysteine to the N- or C-terminus of an albumin protein.

In some embodiments of the invention, the net result of the substitution, deletion, addition, or insertion events of (a), (b), (c) and/or (d) is that the number of conjugation competent cysteine residues of the polypeptide sequence is increased relative to the parent albumin sequence. In some embodiments of the invention, the net result of the substitution, deletion, addition, or insertion events of (a), (b), (c) and/or (d) is that the number of conjugation competent-cysteine residues of the polypeptide sequence is one, thus enabling site-specific conjugation.

Preferred albumin protein variants also include albumin proteins having a single solvent-exposed lysine residue, thus enabling site-specific conjugation of a linker to the lysine residue. Such variants may be generated by engineering an albumin protein, including any of the methods previously described (e.g., insertion, deletion, substitution, or C-terminal or N-terminal fusion).

### Albumin Protein-Binding Peptides

Conjugation of a biologically-active compound to an albumin protein-binding peptide can alter the pharmacodynamics of the biologically-active compound, including the alteration of tissue uptake, penetration, and diffusion. In a preferred embodiment, conjugation of an albumin protein-binding peptide to a compound of the invention (e.g., a RSV F protein inhibitor monomer or dimer, by way of a linker) increases the efficacy or decreases the toxicity of the compound, as compared to the compound alone.

Albumin protein-binding peptides of the invention include any polypeptide having an amino acid sequence of 5 to 50 (e.g., 5 to 40, 5 to 30, 5 to 20, 5 to 15, 5 to 10, 10 to 50, 10 to 30, or 10 to 20) amino acid residues that has affinity for and functions to bind an albumin protein, such as any of the albumin proteins described herein. Preferably, the albumin protein-binding peptide binds to a naturally occurring serum albumin, most preferably human serum albumin. An albumin protein-binding peptide can be of different origins, e.g., synthetic, human, mouse, or rat. Albumin protein-binding peptides of the invention include albumin protein-binding peptides which have been engineered to include one or more (e.g., two, three, four, or five) solvent-exposed cysteine or lysine residues, which may provide a site for conjugation to a compound of the invention (e.g., conjugation to a RSV F protein inhibitor monomer or dimer, including by way of a linker). Most preferably, the albumin protein-binding peptide will contain a single solvent-exposed cysteine or lysine, thus enabling site-specific conjugation of a compound of the invention. Albumin protein-binding peptides may include only naturally occurring amino acid residues, or may include one or more non-naturally occurring amino acid residues. Where included, a non-naturally occurring amino acid residue (e.g., the side chain of a non-naturally occurring amino acid residue) may be used as the point of attachment fora compound of the invention (e.g., a RSV F protein inhibitor monomer or dimer, including by way of a linker). Albumin protein-binding peptides of the invention may be linear or cyclic. Albumin protein-binding peptides of the invention include any albumin protein-binding peptides known to one of skill in the art, examples of which, are provided herein.

Albumin protein-binding peptide, and conjugates including an albumin protein-binding peptide, preferably bind an albumin protein (e.g., human serum albumin) with an affinity characterized by a dissociation constant, Kd, that is less than about 100 μM, preferably less than about 100 nM, and most preferably do not substantially bind other plasma proteins. Specific examples of such compounds are linear or cyclic peptides, preferably between about 10 and 20 amino acid residues in length, optionally modified at the N-terminus or C-terminus or both.

Albumin protein-binding peptides include linear and cyclic peptides including the following general formulae, wherein Xaa is any amino acid:

SEQ ID NO: 101
Xaa-Xaa-Cys-Xaa-Xaa-Xaa-Xaa-Xaa-Cys-Xaa-Xaa-Phe-
Cys-Xaa-Asp-Trp-Pro-Xaa-Xaa-Xaa-Ser-Cys
SEQ ID NO: 102
Val-Cys-Tyr-Xaa-Xaa-Xaa-Ile-Cys-Phe
SEQ ID NO: 103
Cys-Tyr-Xaa-Pro-Gly-Xaa-Cys
SEQ ID NO: 104
Asp-Xaa-Cys-Leu-Pro-Xaa-Trp-Gly-Cys-Leu-Trp
SEQ ID NO: 105
Trp-Cys-Asp-Xaa-Xaa-Leu-Xaa-Ala-Xaa-Asp-Leu-Cys
SEQ ID NO: 106
Asp-Leu-Val-Xaa-Leu-Gly-Leu-Glu-Cys-Trp

Albumin protein-binding peptides of the invention further include any of the following peptide sequences, which may be linear or cyclic:

SEQ ID NO: 107
DLCLRDWGCLW
SEQ ID NO: 108
DICLPRWGCLW
SEQ ID NO: 109
MEDICLPRWGCLWGD
SEQ ID NO: 110
QRLMEDICLPRWGCLWEDDE
SEQ ID NO: 111
QGLIGDICLPRWGCLWGRSV
SEQ ID NO: 112
QGLIGDICLPRWGCLWGRSVK
SEQ ID NO: 113
EDICLPRWGCLWEDD
SEQ ID NO: 114
RLMEDICLPRWGCLWEDD
SEQ ID NO: 115
MEDICLPRWGCLWEDD
SEQ ID NO: 116
MEDICLPRWGCLWED
SEQ ID NO: 117
RLMEDICLARWGCLWEDD
SEQ ID NO: 118
EVRSFCTRWPAEKSCKPLRG
SEQ ID NO: 119
RAPESFVCYWETICFERSEQ
SEQ ID NO: 120
EMCYFPGICWM

Albumin protein-binding peptides of SEQ ID NOs: 101-120 may further include additional amino acids at the N-terminus (Xaa)x and/or additional amino acids at the C-terminus (Xaa)z, wherein Xaa is any amino acid and x and z are a whole number greater or equal to zero, generally less than 100, preferably less than 10 and more preferably 0, 1, 2, 3, 4 or 5.

Further exemplary albumin protein-binding peptides are provided in U.S. Patent Application No. 2005/0287153, which is incorporated herein by reference in its entirety.

### Conjugation of Albumin Protein-Binding Peptides

An albumin protein-binding peptide of the invention may be conjugated to (e.g., by way of a covalent bond) to any compound of the invention (e.g., by way of the linker portion of a RSV F protein inhibitor monomer or dimer). The albumin protein-binding peptide may be conjugated to any compound of the invention by any method known to those of skill in the art for producing peptide-small molecule conjugates. This may include covalent conjugation to the side chain group of an amino acid residue, such as a cysteine, a lysine, or a non-natural amino acid. Alternately, covalent conjugation may occur at the C-terminus (e.g., to the C-terminal carboxylic acid, or to the side chain group of the C-terminal residue) or at the N-terminus (e.g., to the N-terminal amino group, or to the side chain group of the N-terminal amino acid).

### V. Linkers

A linker refers to a linkage or connection between two or more components in a conjugate described herein (e.g., between two RSV F protein inhibitors in a conjugate described herein, between a RSV F protein inhibitor and an Fc domain or an albumin protein in a conjugate described herein, and between a dimer of two RSV F protein inhibitors and an Fc domain or an albumin protein in a conjugate described herein).

### Linkers in Conjugates Having an Fc Domain or an Albumin Protein Covalently Linked to Dimers of RSV F Protein Inhibitors

In a conjugate containing an Fc domain monomer, and Fc domain, an Fc-binding peptide, an albumin protein, or an albumin protein-binding peptide covalently linked to one or more dimers of RSV F protein inhibitors as described herein, a linker in the conjugate (e.g., L or L′) may be a branched structure. As described further herein, a linker in a conjugate described herein (e.g., L or L′) may be a multivalent structure, e.g., a divalent or trivalent structure having two or three arms, respectively. In some embodiments when the linker has three arms, two of the arms may be attached to the first and second RSV F protein inhibitors and the third arm may be attached to the Fc domain monomer, and Fc domain, an Fc-binding peptide, an albumin protein, or an albumin protein-binding peptide. In some embodiments when the linker has two arms, one arm may be attached to an Fc domain or an albumin protein and the other arm may be attached to one of the two RSV F protein inhibitors. In other embodiments, a linker with two arms may be used to attach the two RSV F protein inhibitors on a conjugate containing an Fc domain or albumin protein covalently linked to one or more dimers of RSV F protein inhibitors.

In some embodiments, a linker in a conjugate having an Fc domain or an albumin protein covalently linked to one or more dimers of RSV F protein inhibitors is described by formula (D-L-I):

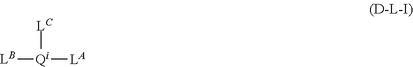

wherein L^{A} is described by formula G^{A1}-(Z^{A1})_{g1}—(Y^{A1})ₕ₁—(Z^{A2})ᵢ₁—(Y^{A2})ⱼ₁—(Z^{A3})ₖ₁—(Y^{A3})ₗ₁—(Z^{A4})ₘ₁—(Y^{A4})ₙ₁—(Z^{A5})_{O1}-G^{A2}; L^{B} is described by formula G^{B1}-(Z^{B1})_{g2}—(Y^{B1})ₕ₂—(Z^{B2})ᵢ₂—(Y^{B2})ⱼ₂—(Z^{B3})ₖ₂—(Y^{B3})ₗ₂—(Z^{B4})ₘ₂—(Y^{B4})ₙ₂—(Z^{B5})_{O2}-G^{B2}; L^{C} is described by formula G^{C1}-(Z^{C1})_{g3}—(Y^{C1})ₕ₃—(Z^{C2})ᵢ₃—(Y^{C2})ⱼ₃—(Z^{C3})ₖ₃—(Y^{C3})ₗ₃—(Z^{C4})ₘ₃—(Y^{C4})ₙ₃—(Z^{C5})_{O3}-G^{C2}; G^{A1} is a bond attached to Qⁱ in formula (D-L-I); G^{A2} is a bond attached to the first RSV F protein inhibitor (e.g., A₁); G^{B1} is a bond attached to Qⁱ in formula (D-L-I); G^{B2} is a bond attached to the second RSV F protein inhibitor (e.g., A2); G^{C1} is a bond attached to Qⁱ in formula (D-L-I); G^{C2} is a bond attached to an Fc domain monomer, and Fc domain, an Fc-binding peptide, an albumin protein, or an albumin protein-binding peptide, or a functional group capable of reacting with a functional group conjugated to E (e.g., maleimide and cysteine, amine and activated carboxylic acid, thiol and maleimide, activated sulfonic acid and amine, isocyanate and amine, azide and alkyne, and alkene and tetrazine); each of Z^{A1}, Z^{A2}, Z^{A3}, Z^{A4}, Z^{A5}, Z^{B1}, Z^{B2}, Z^{B3}, Z^{B4}, Z^{B5}, Z^{C1}, Z^{C2}, Z^{C3}, Z^{C4}, and Z^{C5} is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of Y^{A1}, Y^{A2}, Y^{A3}, Y^{A4}, Y^{B1}, Y^{B2}, Y^{B3}, Y^{B4}, Y^{C1}, Y^{C2}, Y^{C3}, and Y^{C4} is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C2-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; each of g1, h1, i1, j1, k1, l1, m1, n1, o1, g2, h2, i2, j2, k2, l2, m2, n2, o2, g3, h3, i3, j3, k3, l3, m3, n3, and o3 is, independently, 0 or 1; Q is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C2-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

In some embodiments, optionally substituted includes substitution with a PEG. A PEG has a repeating unit structure (—CH₂CH₂O—)ₙ, wherein n is an integer from 2 to 100. A polyethylene glycol may selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀).

In some embodiments, L^{C} may have two points of attachment to the Fc domain (e.g., two G^{C2}).

In some embodiments, L includes a polyethylene glycol (PEG) linker. A PEG linker includes a linker having the repeating unit structure (—CH₂CH₂O—)ₙ, where n is an integer from 2 to 100. A polyethylene glycol linker may covalently join a RSV F protein inhibitor and E (e.g., in a conjugate of any one of formulas (M-I)-(M-IV)). A polyethylene glycol linker may covalently join a first RSV F protein inhibitor and a second RSV F protein inhibitor (e.g., in a conjugate of any one of formulas (D-I)-(D-IV)). A polyethylene glycol linker may covalently join a RSV F protein inhibitor dimer and E (e.g., in a conjugate of any one of formulas (D-I)-(D-IV)). A polyethylene glycol linker may selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀). In some embodiments, L^{c} includes a PEG linker, where L^{C} is covalently attached to each of Qⁱ and E.

Linkers of formula (D-L-I) that may be used in conjugates described herein include, but are not limited to

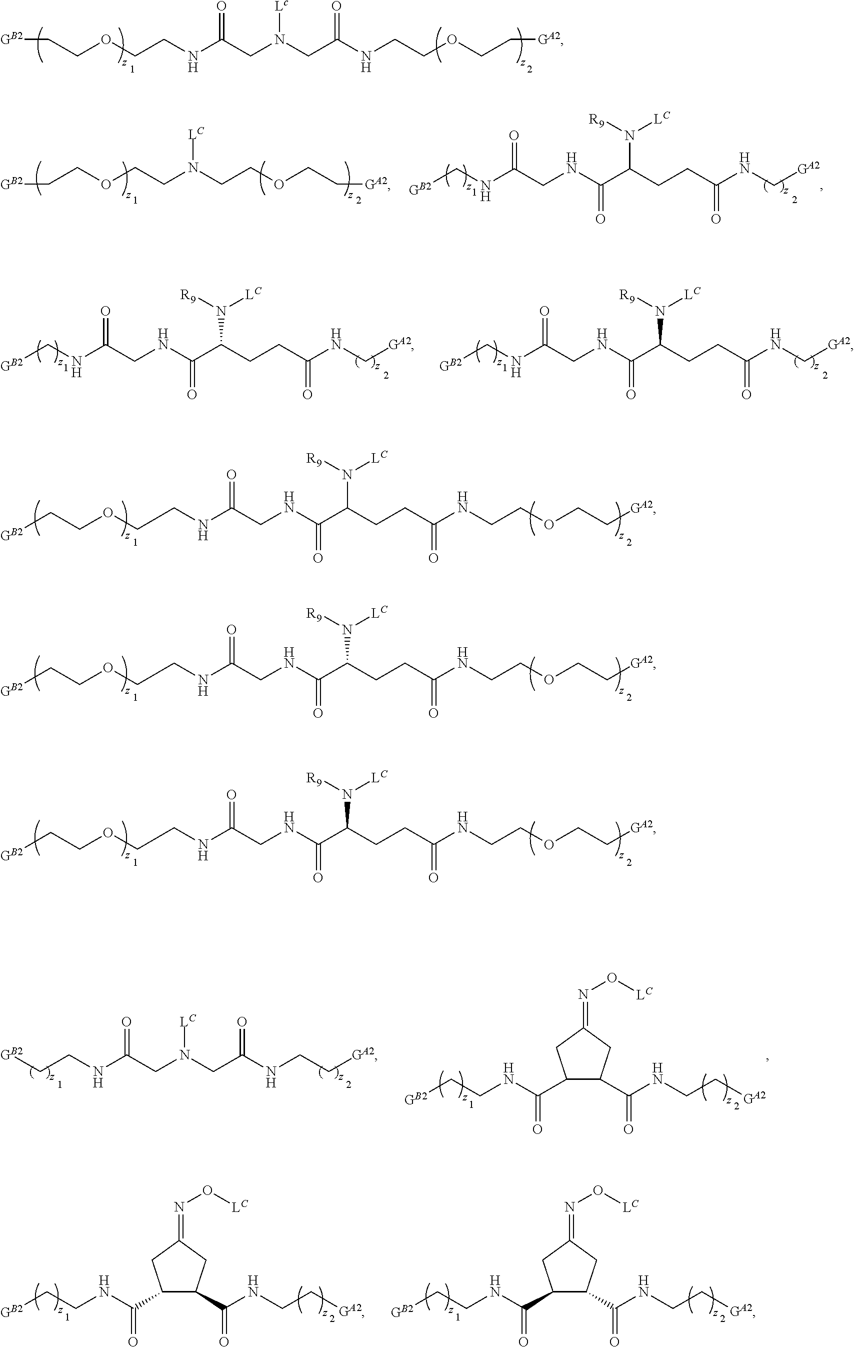

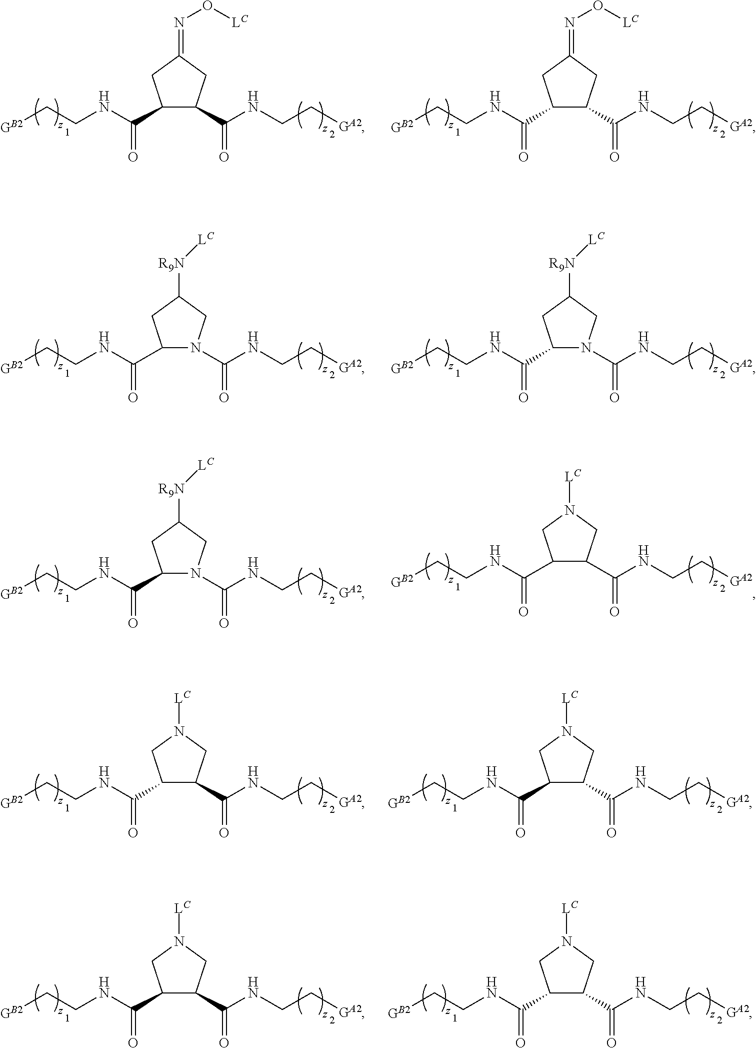

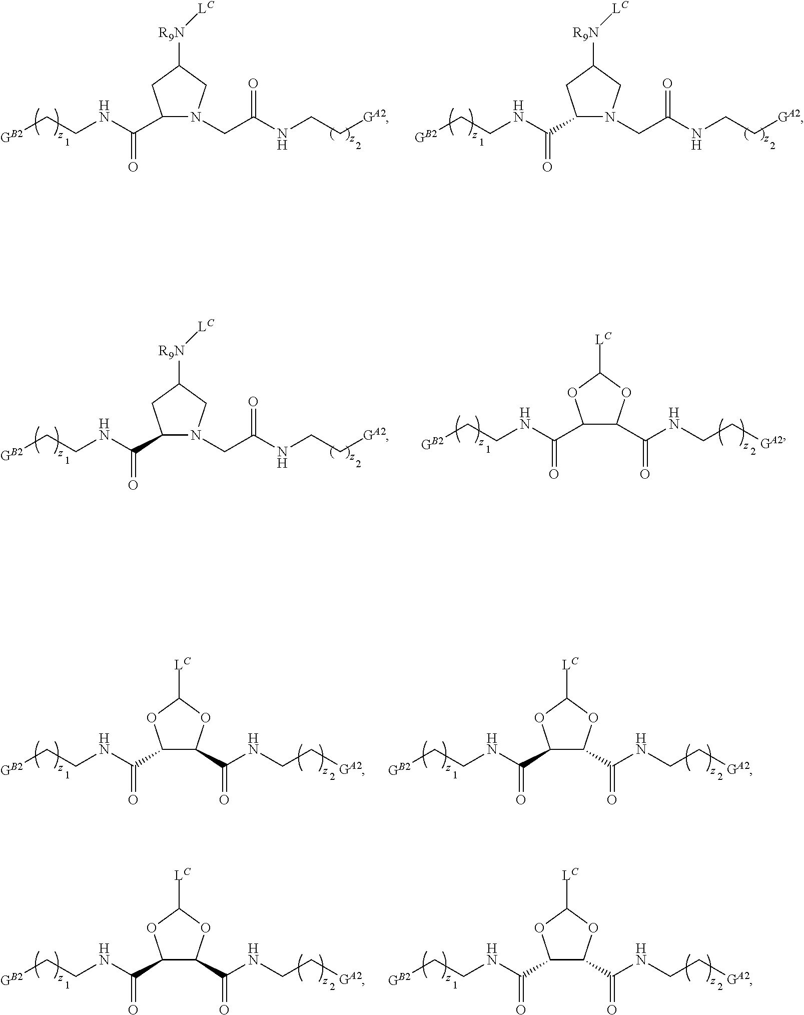

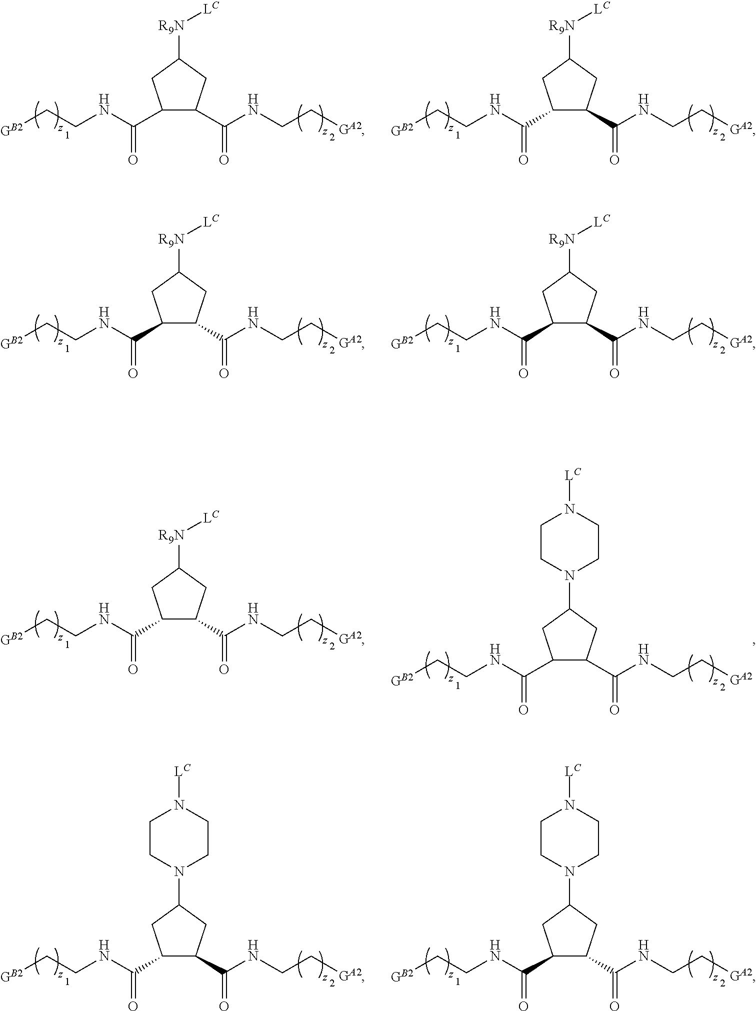

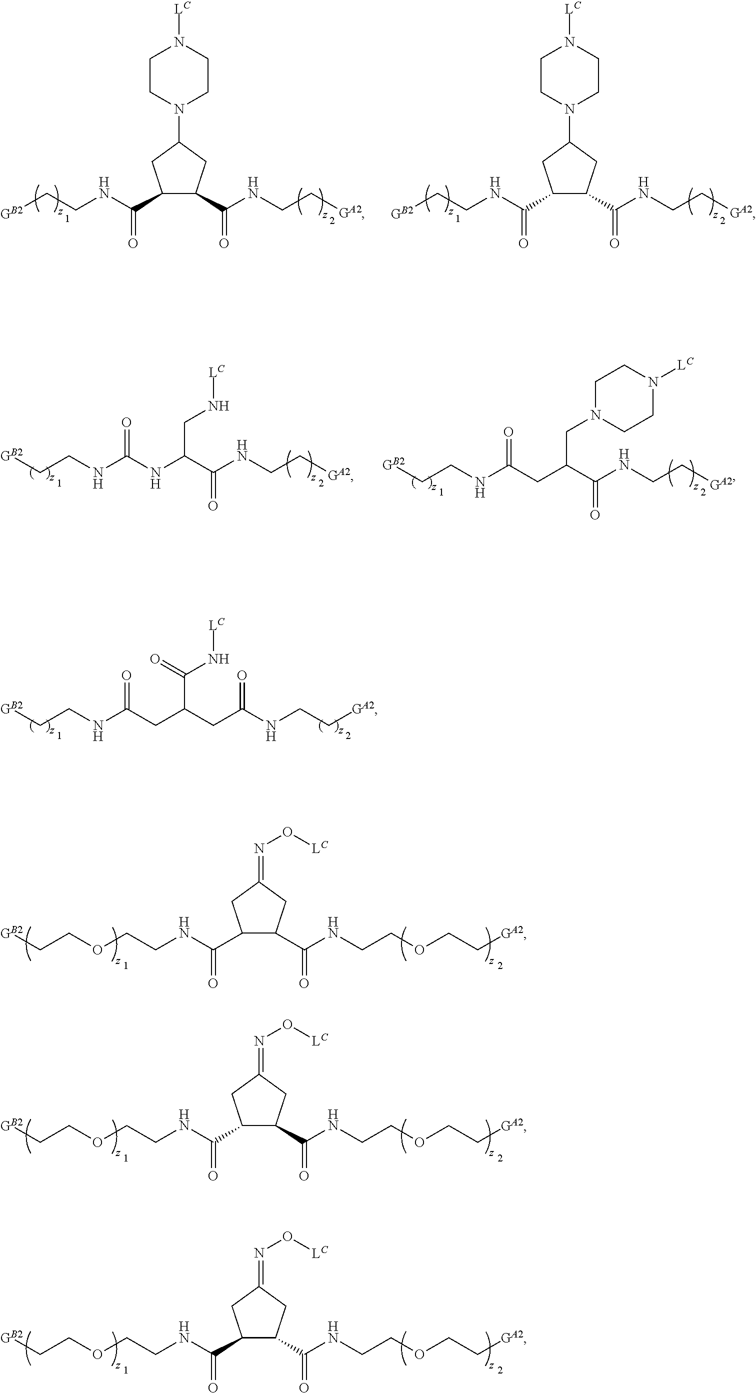

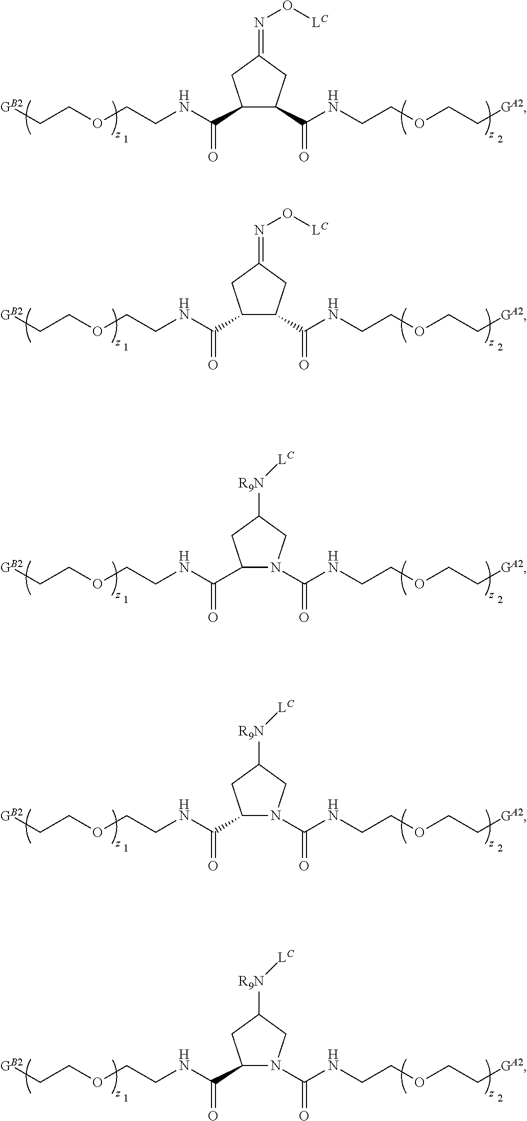

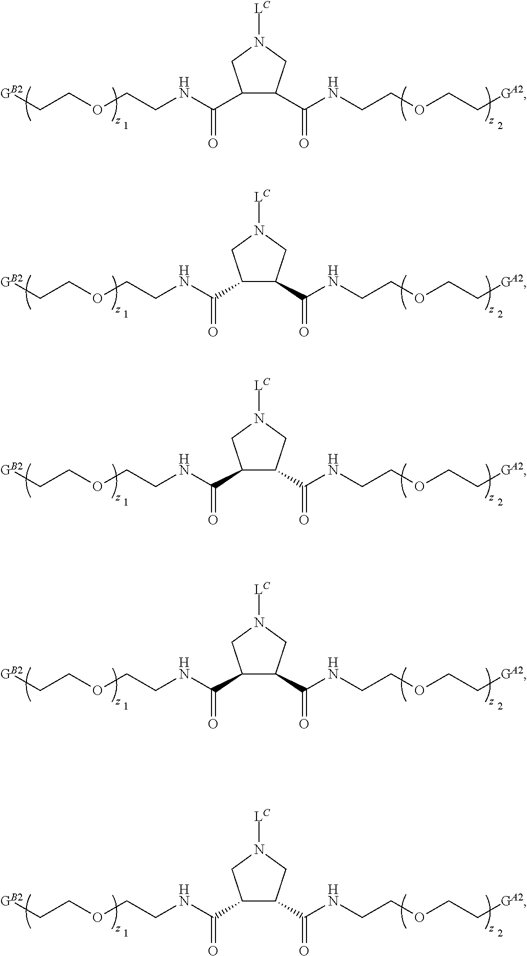

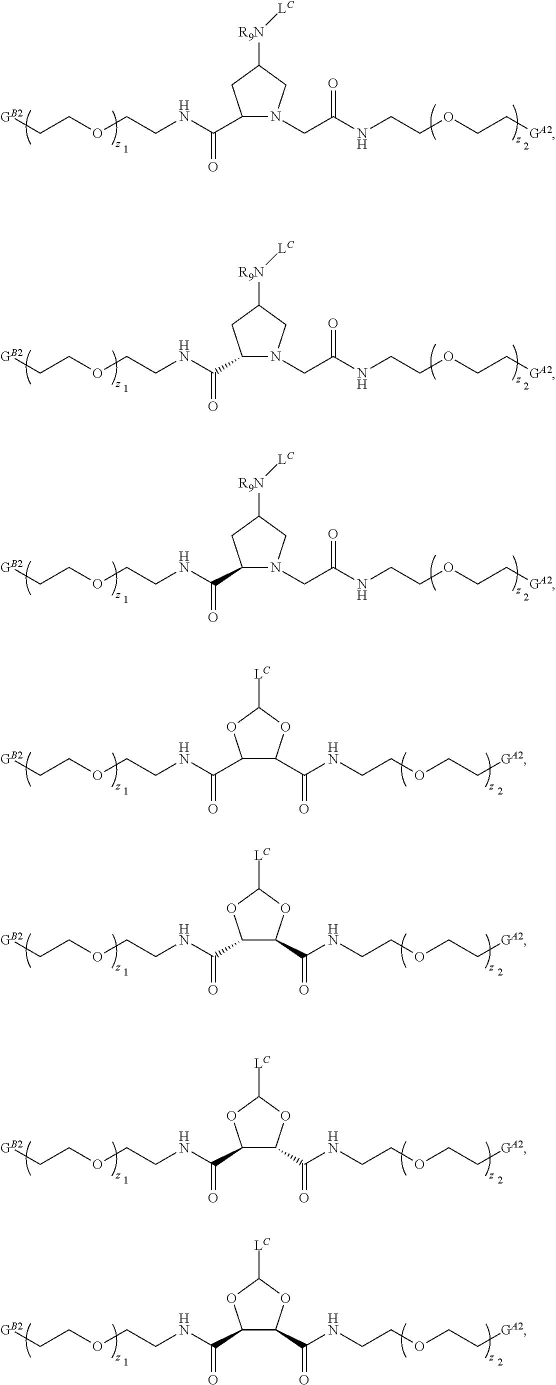

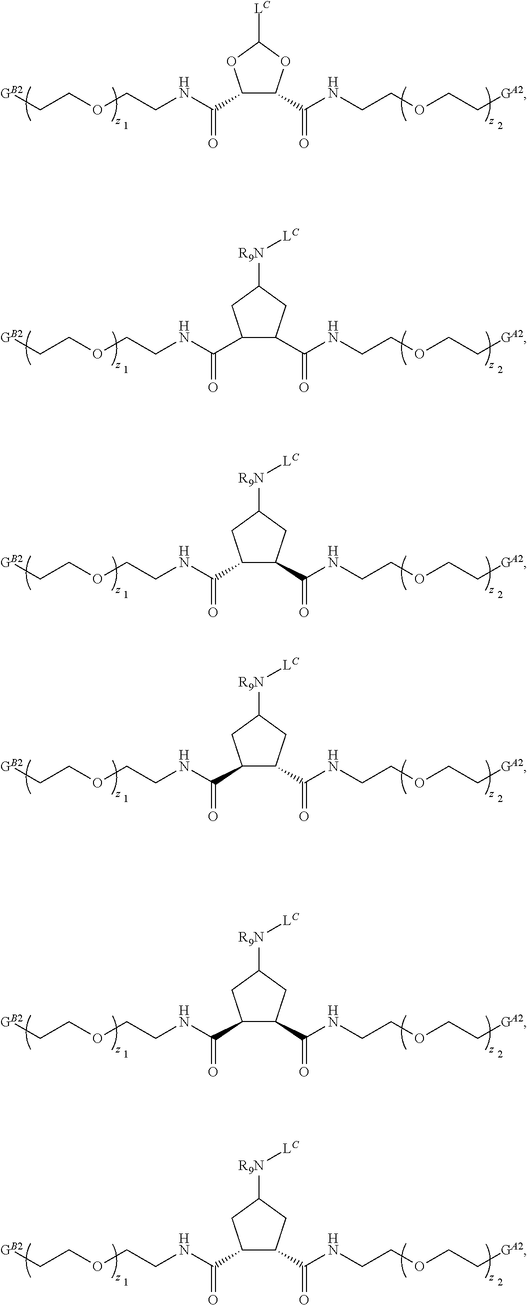

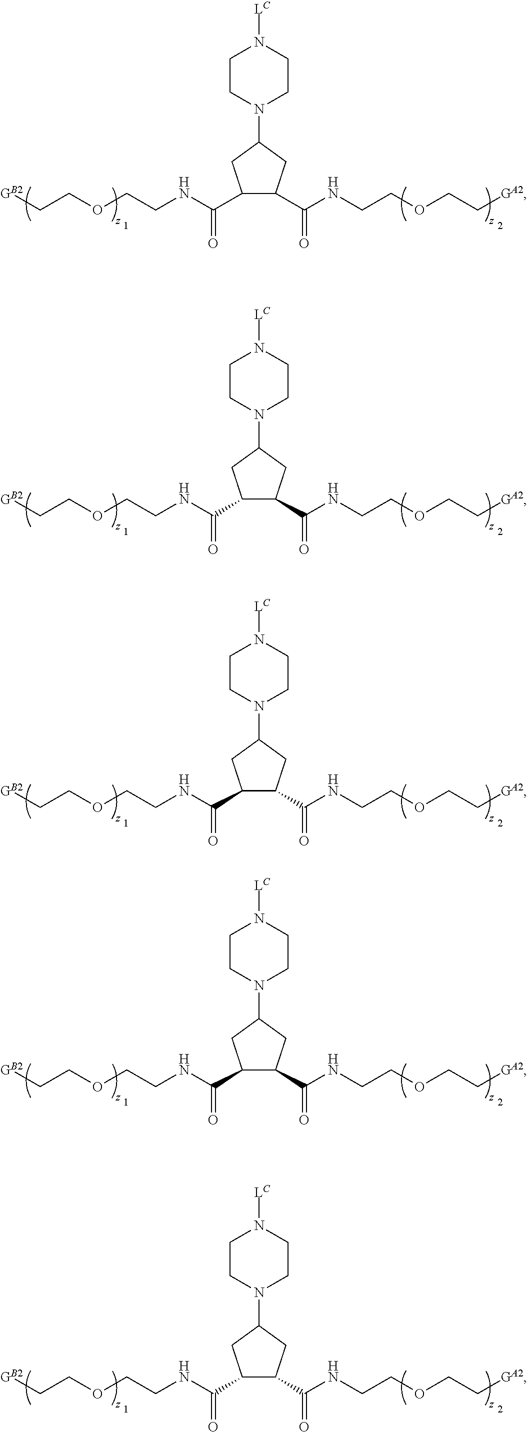

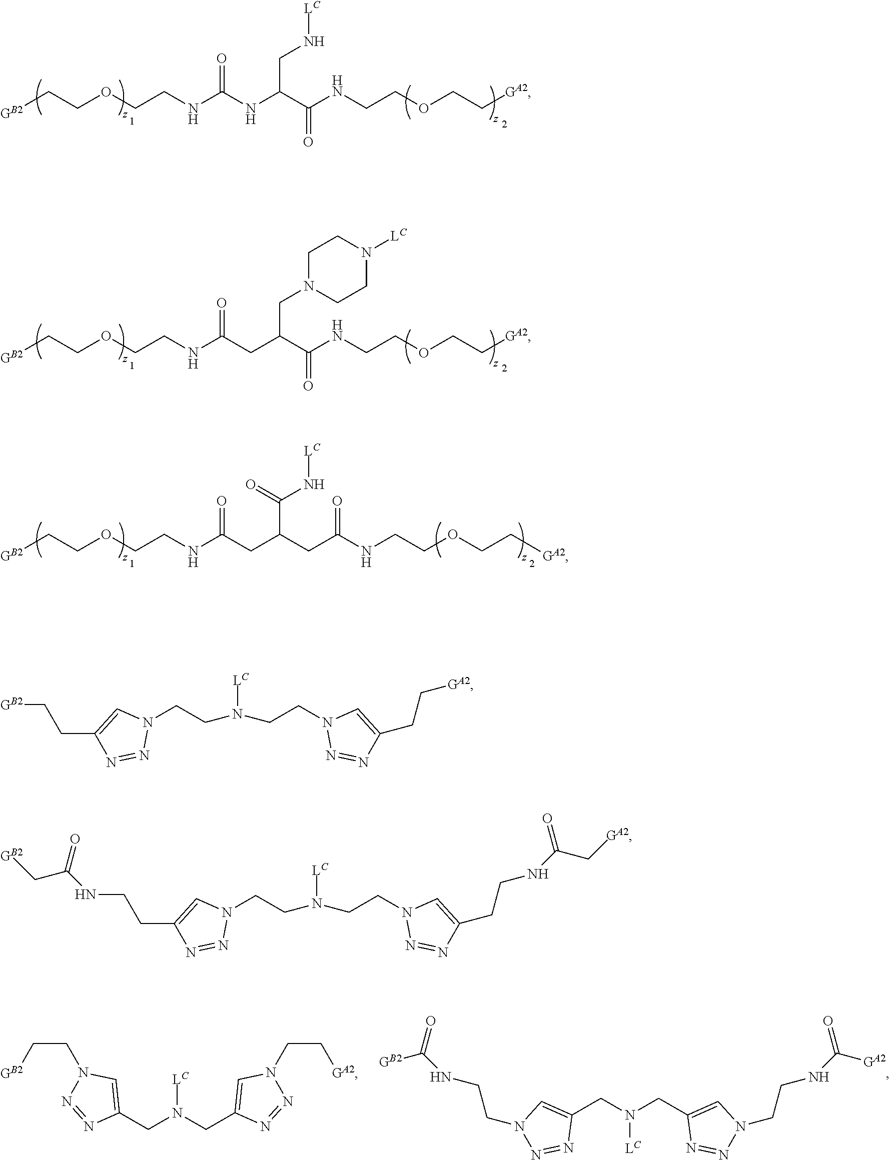

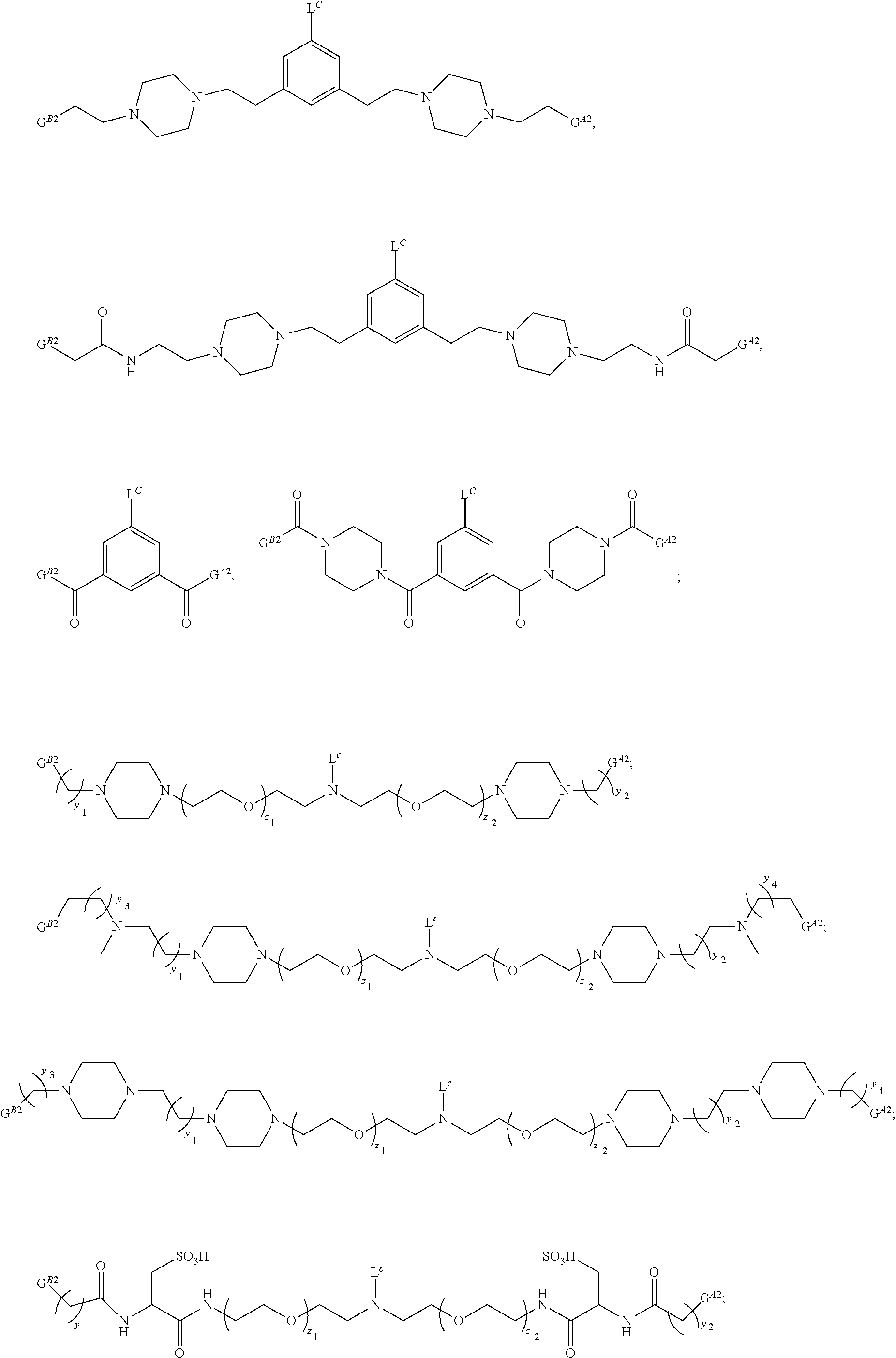

where z₁ and z₂ are each, independently, and integer from 1 to 20; and R₉ is selected from H, C1-C20 alkyl, C3-C20 cycloalkyl, C₂-C₂₀ heterocycloalkyl; C5-C15 aryl, and C2-C15 heteroaryl.

Linkers of the formula (D-L-I) may also include any of

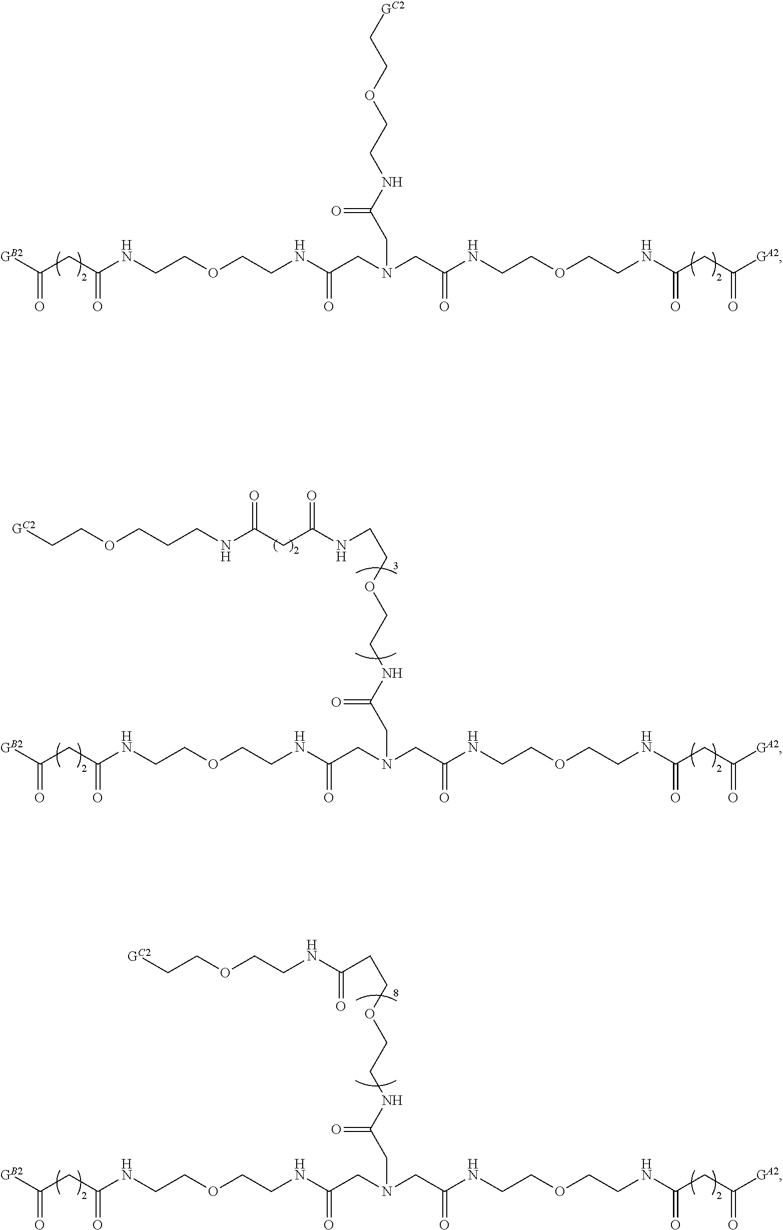

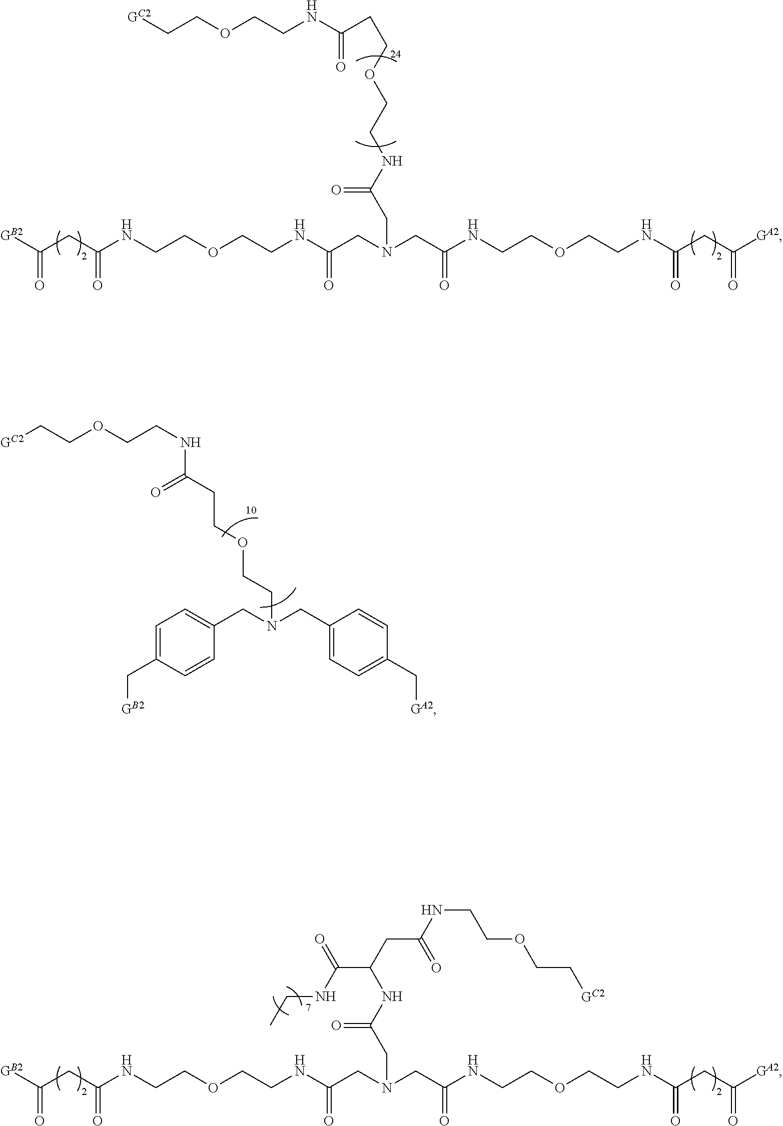

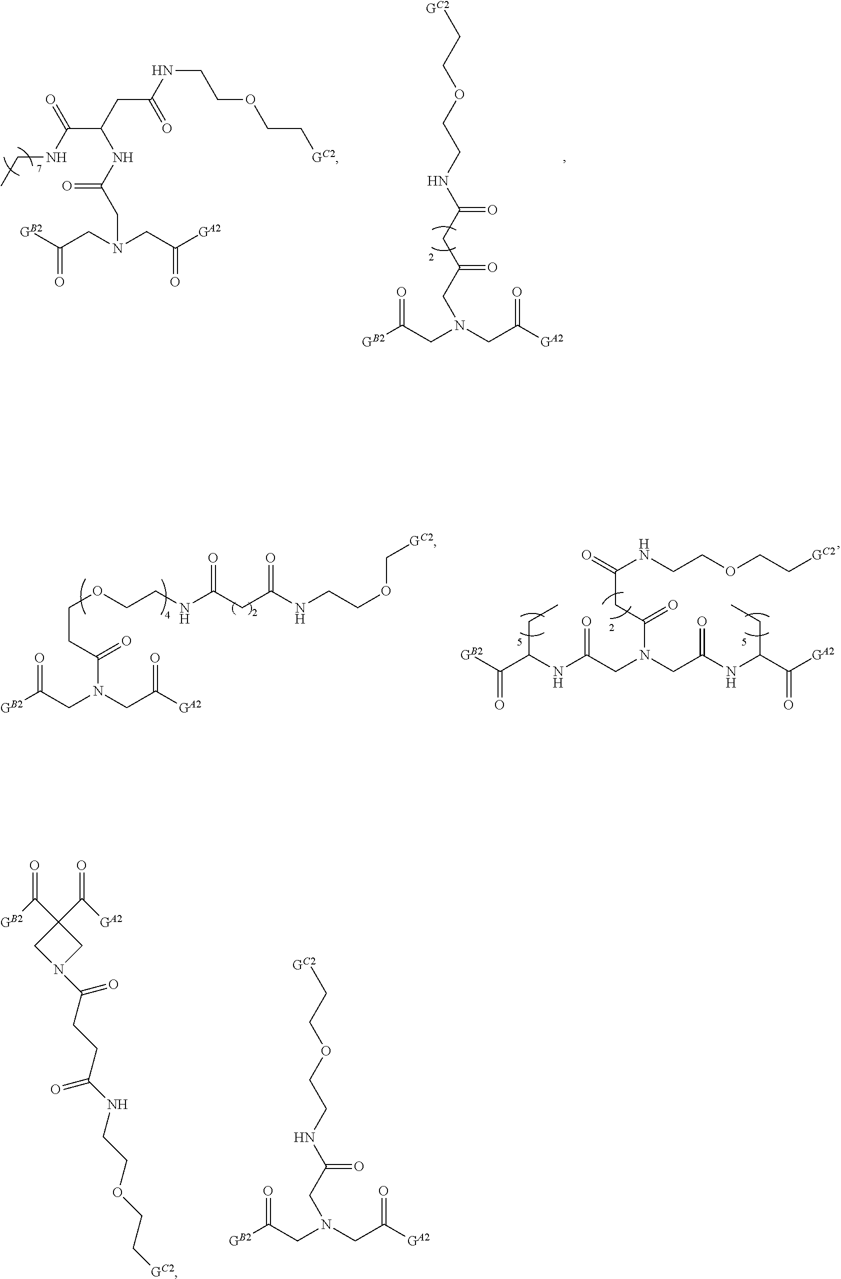

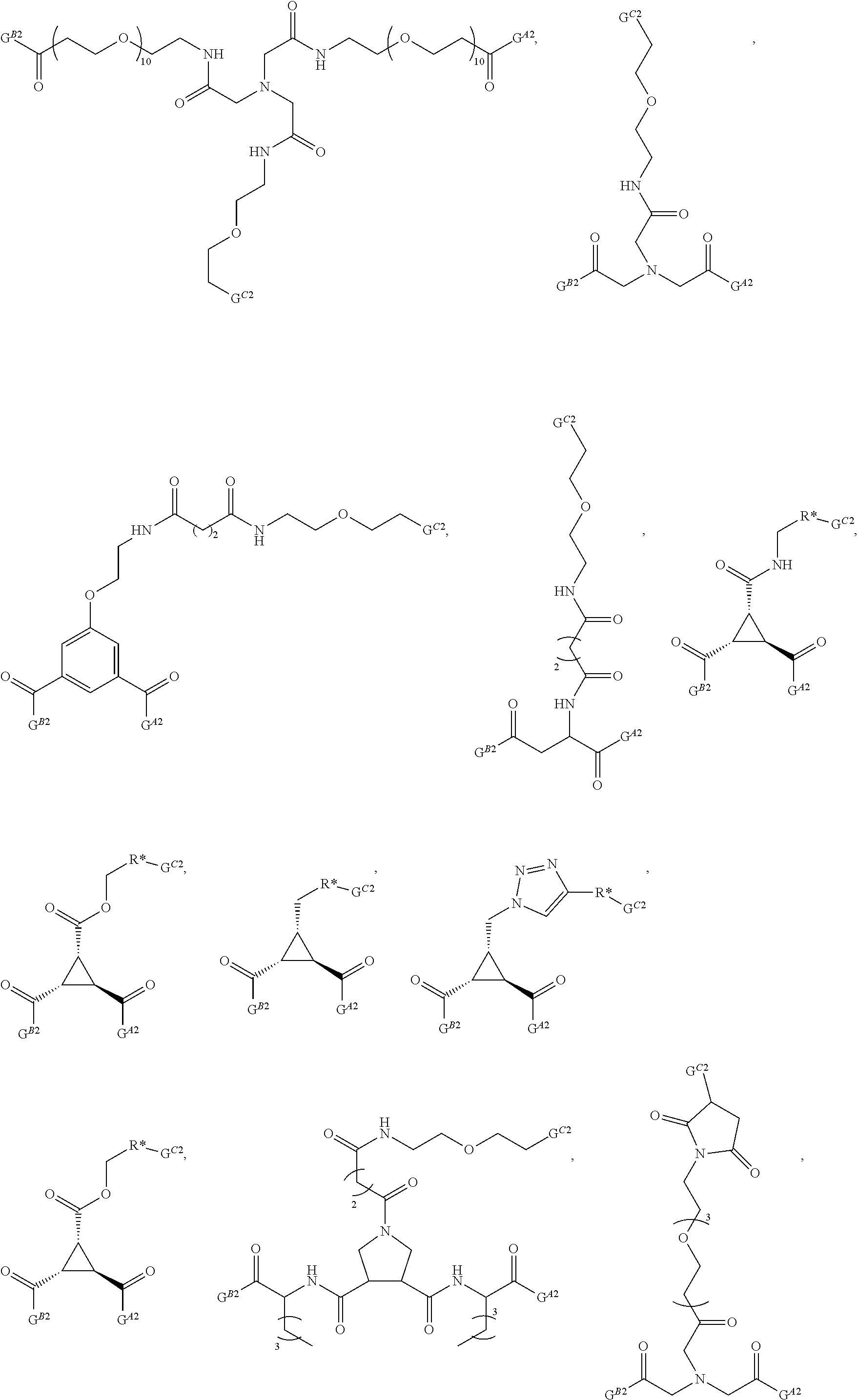

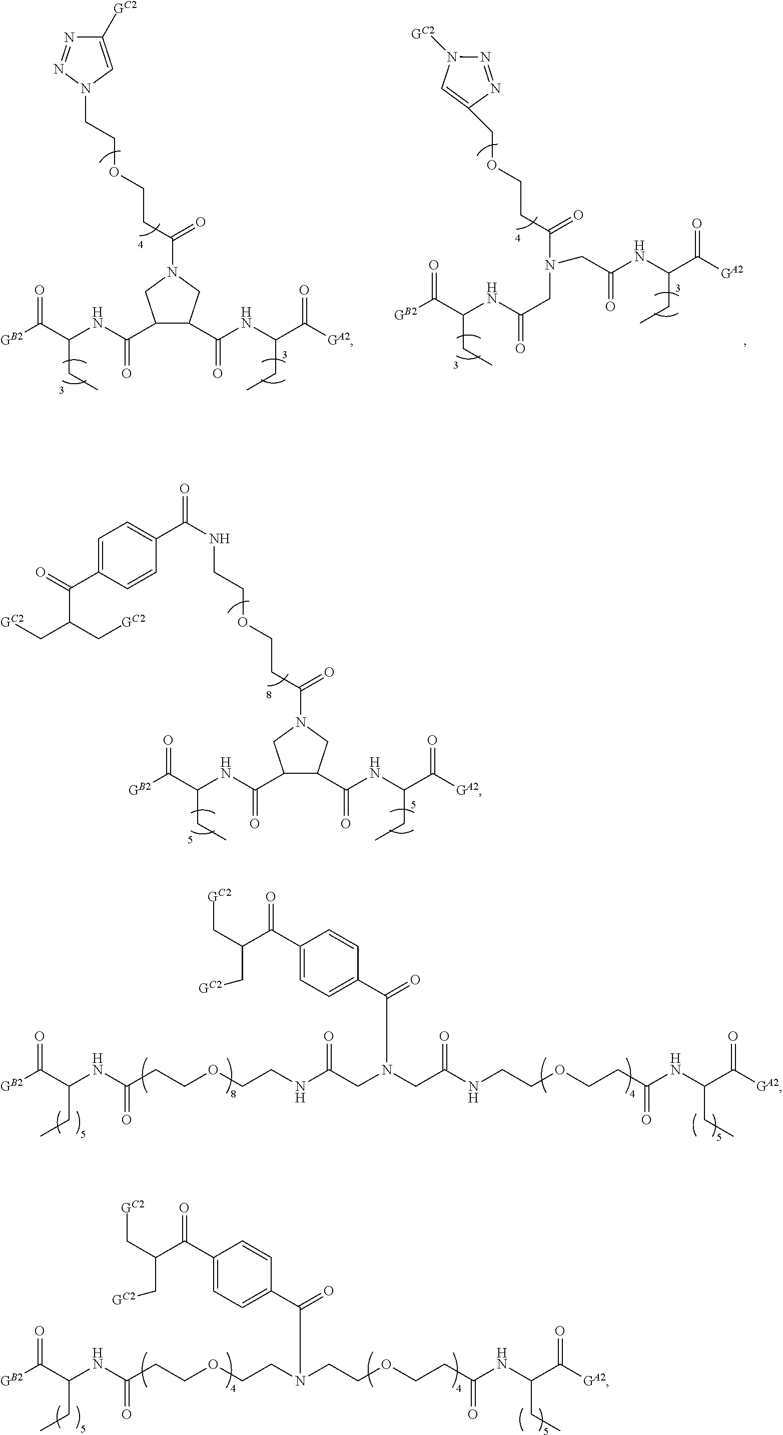

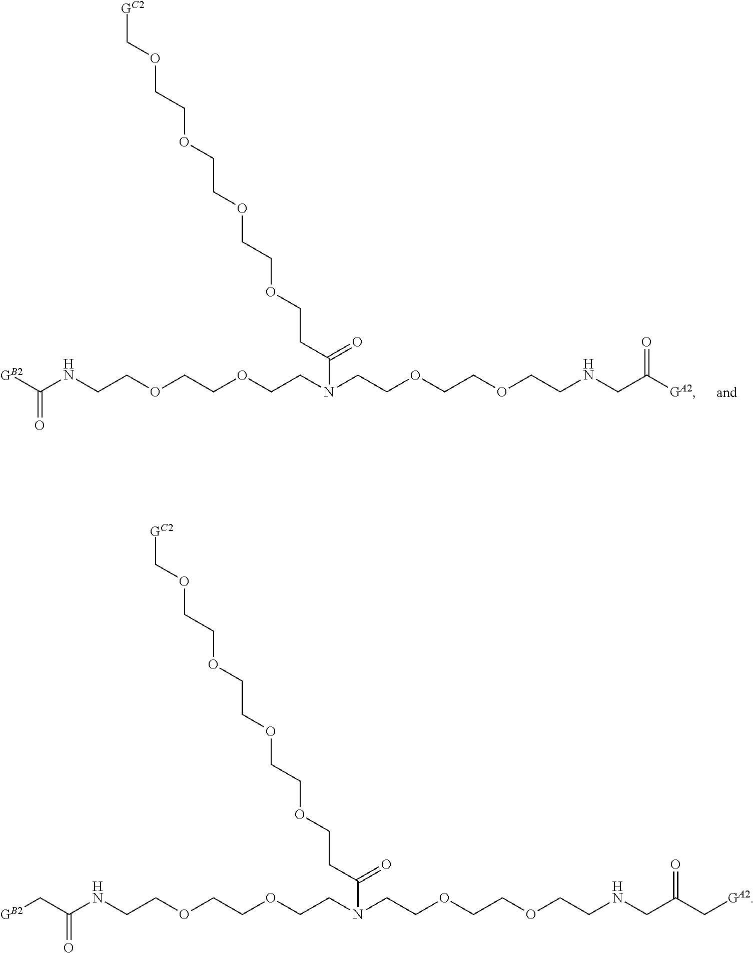

### Linkers in Conjugates Having an Fc Domain or an Albumin Protein Covalently Linked to Monomers of RSV F Protein Inhibitors

In a conjugate containing an Fc domain monomer, and Fc domain, an Fc-binding peptide, an albumin protein, or an albumin protein-binding peptide covalently linked to one or more monomers of RSV F protein inhibitors as described herein, a linker in the conjugate (e.g., L, or L′) may be a divalent structure having two arms. One arm in a divalent linker may be attached to the monomer of RSV F protein inhibitor and the other arm may be attached to the Fc domain monomer, and Fc domain, an Fc-binding peptide, an albumin protein, or an albumin protein-binding peptide. In some embodiments, the one or more monomers of RSV F protein inhibitors in the conjugates described herein may each be, independently, connected to an atom in the Fc domain monomer, and Fc domain, an Fc-binding peptide, an albumin protein, or an albumin protein-binding peptide.

In some embodiments, a linker is described by formula (M-L-I):

J¹-(Q¹)_{g}-(T¹)ₕ-(Q²)ᵢ-(T²)ⱼ-(Q³)ₖ-(T³)ₗ-(Q⁴)ₘ-(T⁴)ₙ-(Q⁵)ₒ-J²

wherein J¹ is a bond attached to a RSV F protein inhibitor; J² is a bond attached to an Fc domain monomer, and Fc domain, an Fc-binding peptide, an albumin protein, or an albumin protein-binding peptide, or a functional group capable of reacting with a functional group conjugated to an Fc domain monomer, an Fc domain, an Fc-binding peptide, an albumin protein, or an albumin protein-binding peptide (e.g., maleimide and cysteine, amine and activated carboxylic acid, thiol and maleimide, activated sulfonic acid and amine, isocyanate and amine, azide and alkyne, and alkene and tetrazene); each of Q¹, Q², Q³, Q⁴, and Q⁵ is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of T¹, T², T³, T⁴ is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and each of g, h, i, j, k, l, m, n, and o is, independently, 0 or 1.

In some embodiments, optionally substituted includes substitution with a polyethylene glycol (PEG). A PEG has a repeating unit structure (—CH₂CH₂O—)ₙ, wherein n is an integer from 2 to 100. A polyethylene glycol may selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀).

In some embodiments, J² may have two points of attachment to the Fc domain monomer, and Fc domain, an Fc-binding peptide, an albumin protein, or an albumin protein-binding peptide (e.g., two J²).

Linkers of formula (M-L-I) that may be used in conjugates described herein include, but are not limited to,

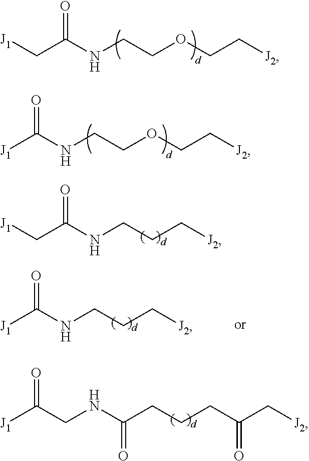

wherein d is an integer from 1 to 20 (e.g., d is 1, 2, 3, 4, 5, 6, 7, 8, 9, 10, 11, 12, 13, 14, 15, 16, 17, 18, 19, or 20).

Linkers of formula (M-L-I) that may be used in conjugates described herein include, but are not limited to,

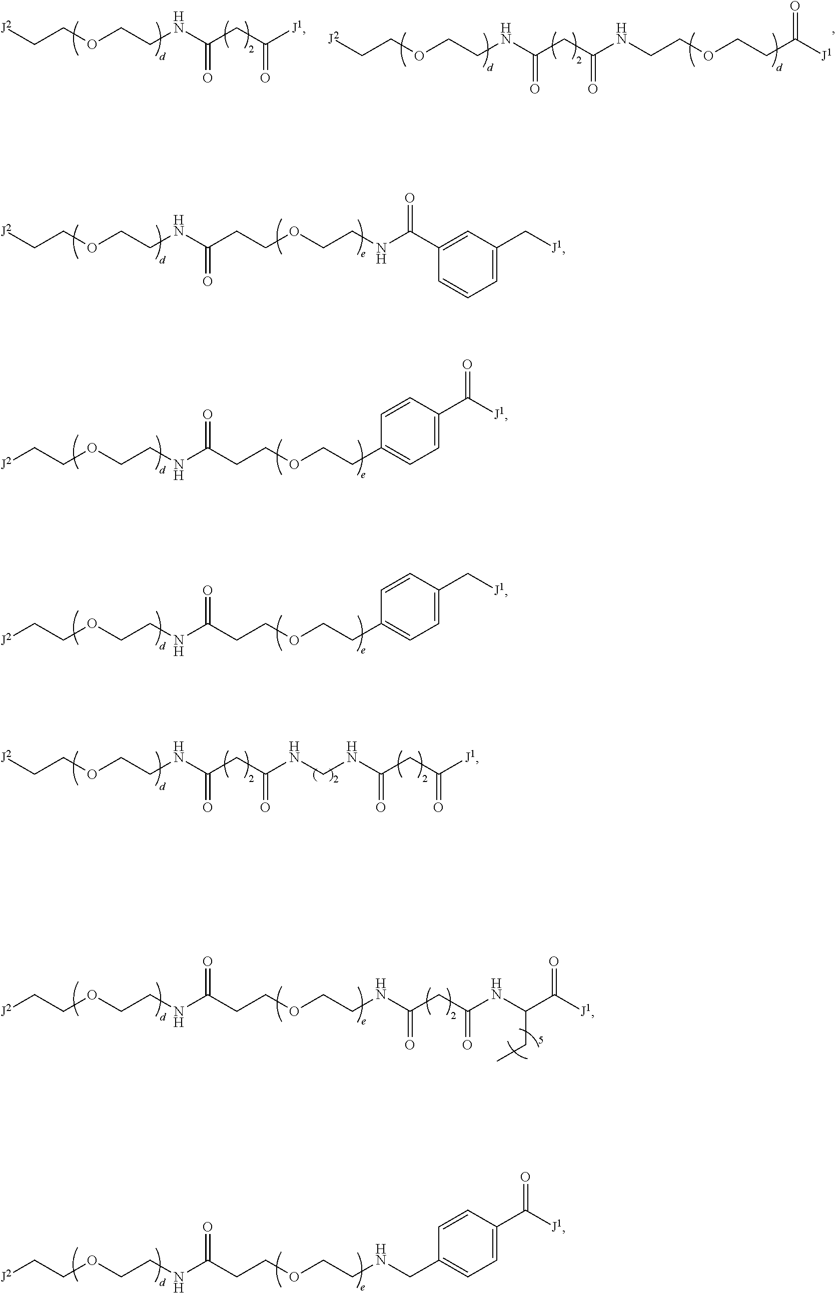

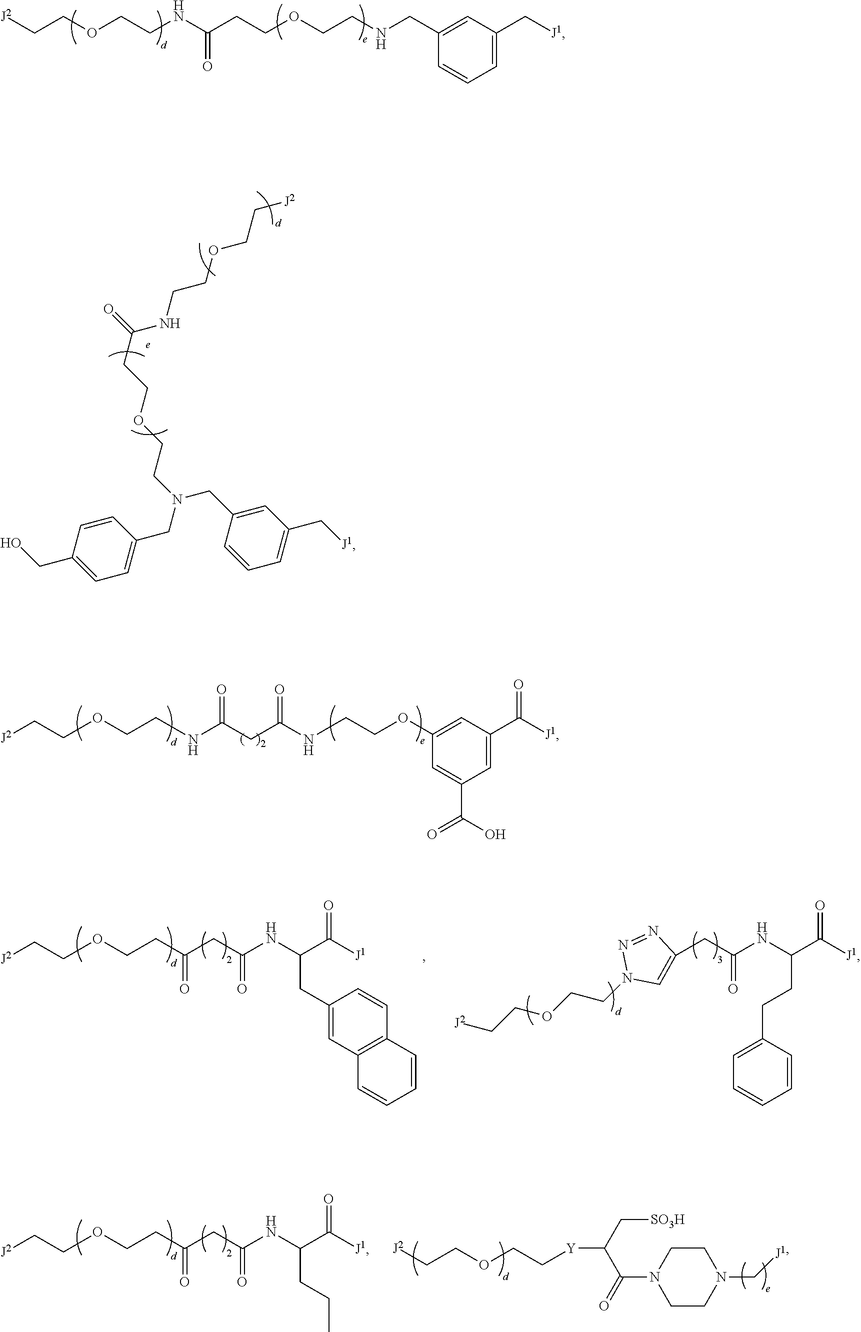

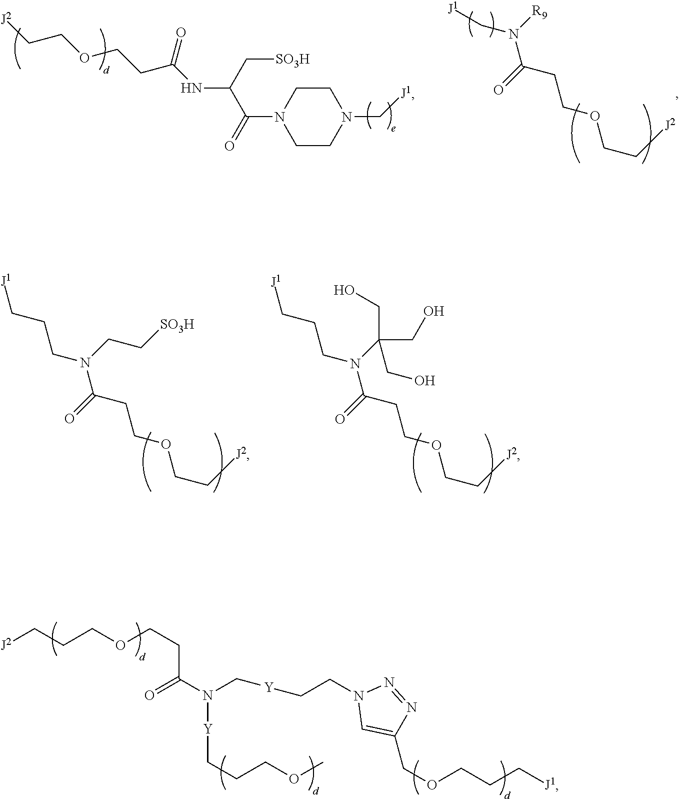

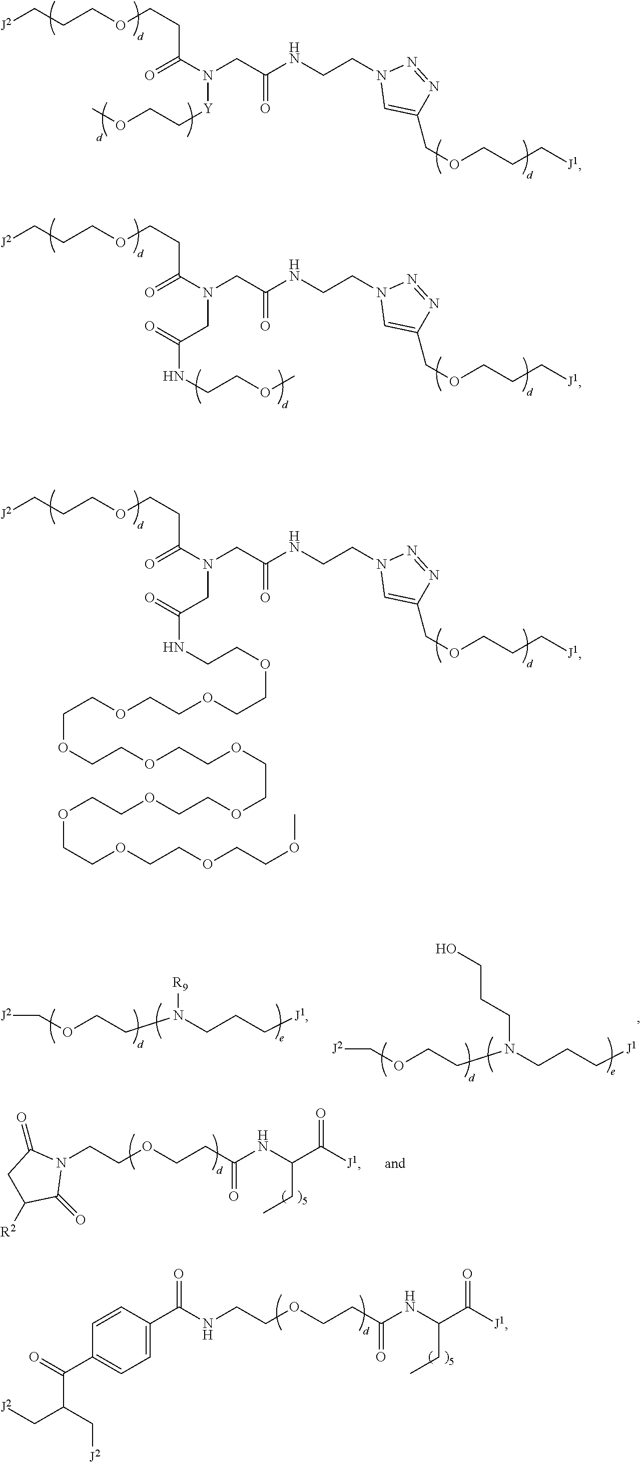

wherein each Y is independently selected from —O—, —S—, —R₈—, (—O(C═O)NR₈—), (—O(C═S)NR₈—), (—O(C═O)O—), (—O(C═O)—), (—NH(C═O)O—), (—NH(C═O)—), (—NH(C═NH)—), (—NH(C═O)NR₈—), (—NH(C═NH)NR₈—), (—NH(C═S)NR₈—), (—NH(C═S)—), (—OCH₂(C═O)NR₈—), (—NH(SO₂)—), (—NH(SO₂)NR₈—), (—OR₉—), (—NR₉—), (—SR₉—), (—R₉NH(C═O)—), (—R₉OR₉C(═O)NH—), (—CH₂NH(C═O)—), (—CH₂OCH₂(C═O)NH—), (—(C═NR₈)NH—), (—NH(SO₂)—), (—(C═O)NH—), (—C(═O)—), (—C(NR₈)—), or (—R₉C(═O)—);

each R₈ is independently selected from H, optionally substituted C₁-C₂₀ alkyl, optionally substituted C₁-C₂₀ alkylene, optionally substituted C₃-C₂₀ cycloalkyl, optionally substituted C₂-C₂₀ heterocycloalkyl, optionally substituted C₅-C₁₅ aryl, and optionally substituted C₂-C₁₅ heteroaryl; each Rs is independently selected from optionally substituted C₁-C₂₀ alkylene, optionally substituted C₃-C₂₀ cycloalkyl, optionally substituted C₂-C₂₀ heterocycloalkyl, optionally substituted C₅-C₁₅ aryl, and optionally substituted C₂-C₁₅ heteroaryl; and

each of d, e, y₁, and x₁ is, independently, an integer from 1 to 26 (e.g., d is 1, 2, 3, 4, 5, 6, 7, 8, 9, 10, 11, 12, 13, 14, 15, 16, 17, 18, 19, 20, 21, 22, 23, 24, 25, or 26).

### Linking Groups

In some embodiments, a linker provides space, rigidity, and/or flexibility between the RSV F protein inhibitors and the Fc domain monomer, and Fc domain, an Fc-binding peptide, an albumin protein, or an albumin protein-binding peptide in the conjugates described here or between two RSV F protein inhibitors in the conjugates described herein. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation. In some embodiments, a linker (L or L′ as shown in any one of formulas (1), (2), (D-I)-(D-IV), or (M-I)-(M-IV) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker (L or L) includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker (L or L) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). The “backbone” of a linker refers to the atoms in the linker that together form the shortest path from one part of the conjugate to another part of the conjugate. The atoms in the backbone of the linker are directly involved in linking one part of the conjugate to another part of the conjugate. For examples, hydrogen atoms attached to carbons in the backbone of the linker are not considered as directly involved in linking one part of the conjugate to another part of the conjugate.

Molecules that may be used to make linkers (L or L′) include at least two functional groups, e.g., two carboxylic acid groups. In some embodiments of a trivalent linker, two arms of a linker may contain two dicarboxylic acids, in which the first carboxylic acid may form a covalent linkage with the first RSV F protein inhibitor in the conjugate and the second carboxylic acid may form a covalent linkage with the second RSV F protein inhibitor in the conjugate, and the third arm of the linker may for a covalent linkage (e.g., a C—O bond) with an Fc domain monomer, and Fc domain, an Fc-binding peptide, an albumin protein, or an albumin protein-binding peptide in the conjugate. In some embodiments of a divalent linker, the divalent linker may contain two carboxylic acids, in which the first carboxylic acid may form a covalent linkage with one component (e.g., a RSV F protein inhibitor) in the conjugate and the second carboxylic acid may form a covalent linkage (e.g., a C—S bond or a C—N bond) with another component (e.g., an Fc domain monomer, and Fc domain, an Fc-binding peptide, an albumin protein, or an albumin protein-binding peptide) in the conjugate.

In some embodiments, dicarboxylic acid molecules may be used as linkers (e.g., a dicarboxylic acid linker). For example, in a conjugate containing an Fc domain monomer, and Fc domain, an Fc-binding peptide, an albumin protein, or an albumin protein-binding peptide covalently linked to one or more dimers of RSV F protein inhibitors, the first carboxylic acid in a dicarboxylic acid molecule may form a covalent linkage with a hydroxyl or amine group of the first RSV F protein inhibitor and the second carboxylic acid may form a covalent linkage with a hydroxyl or amine group of the second RSV F protein inhibitor.

Examples of dicarboxylic acids molecules that may be used to form linkers include, but are not limited to,

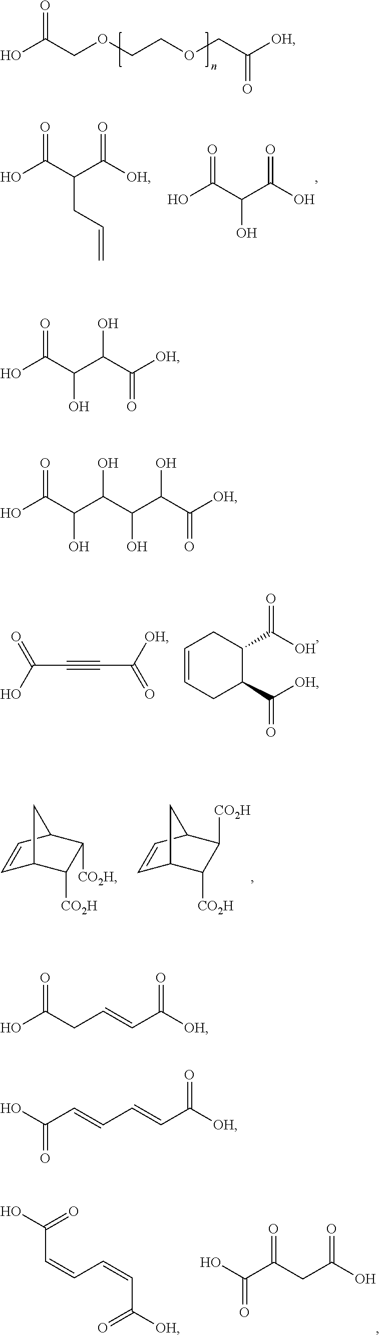

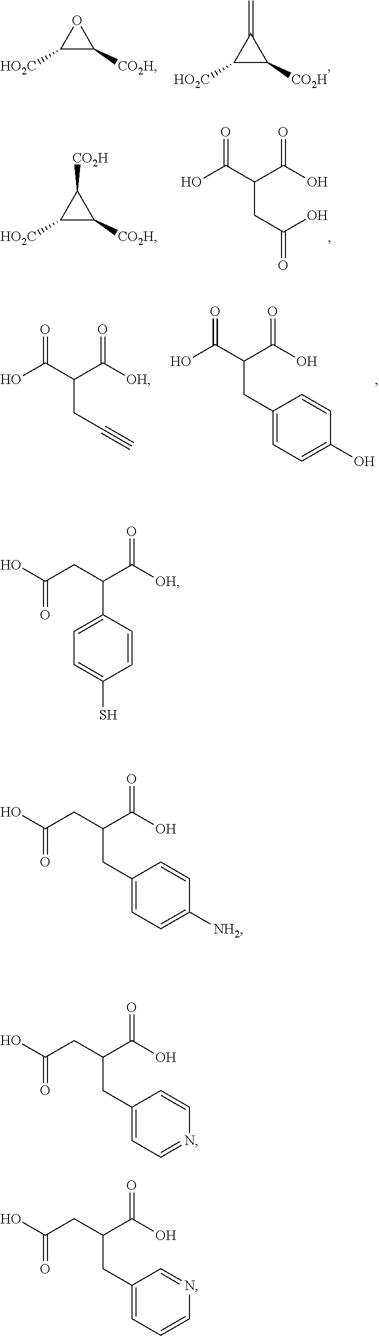

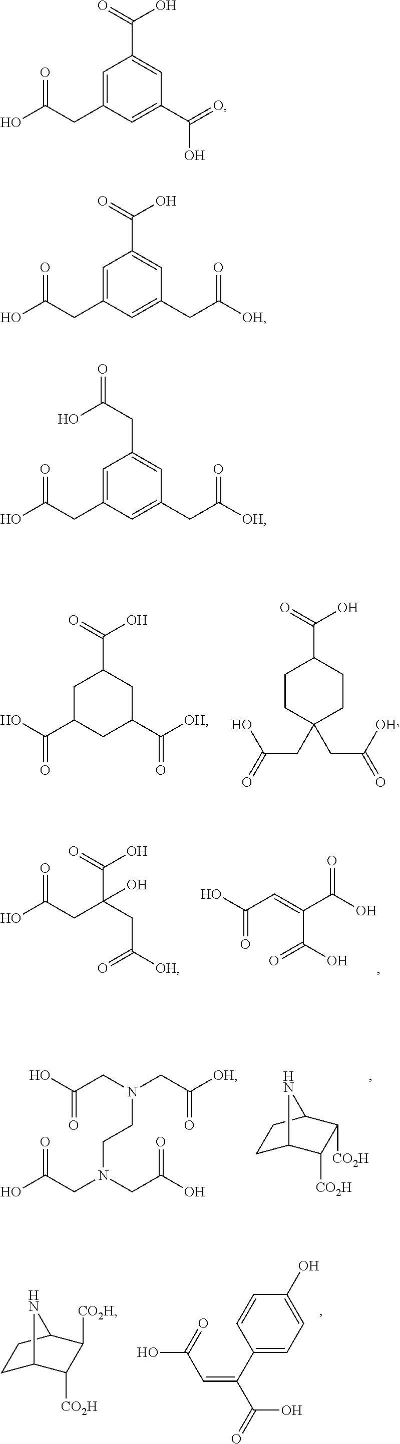

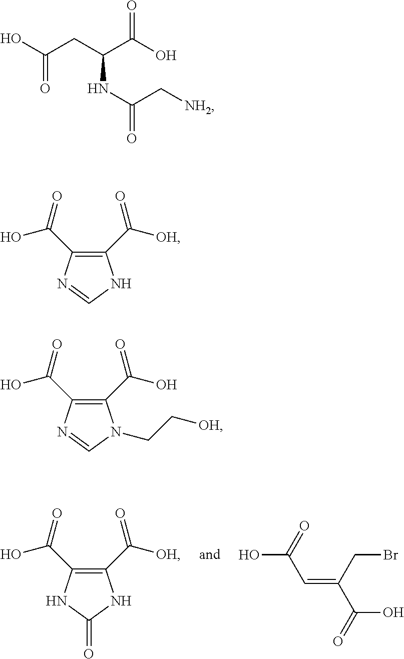

wherein n is an integer from 1 to 20 (e.g., n is 1, 2, 3, 4, 5, 6, 7, 8, 9, 10, 11, 12, 13, 14, 15, 16, 17, 18, 19, or 20).

Other examples of dicarboxylic acids molecules that may be used to form linkers include, but are not limited to,

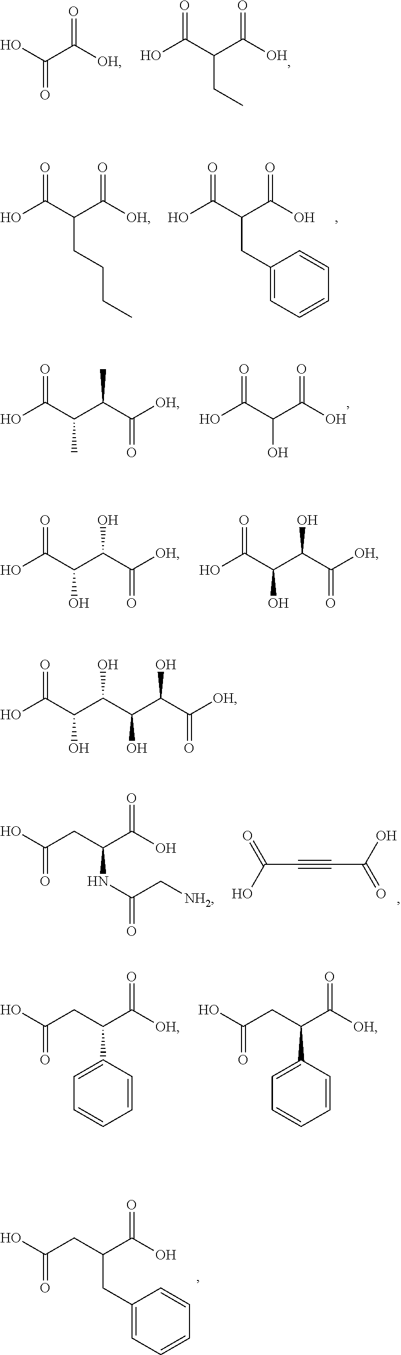

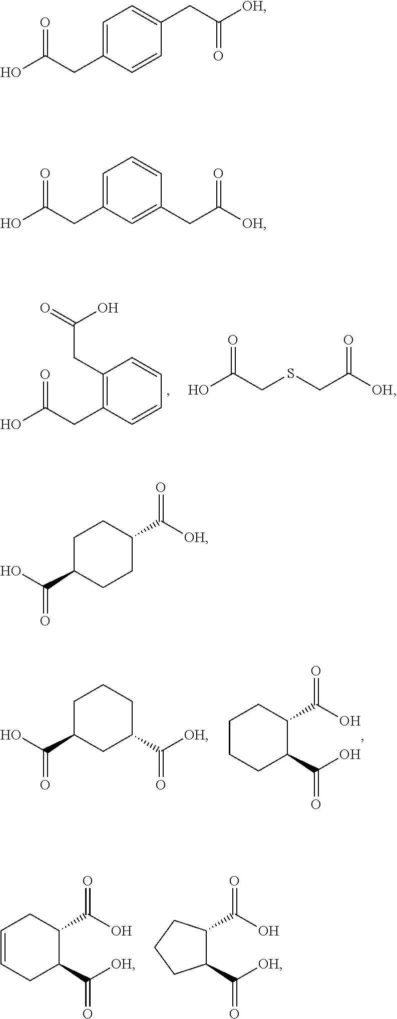

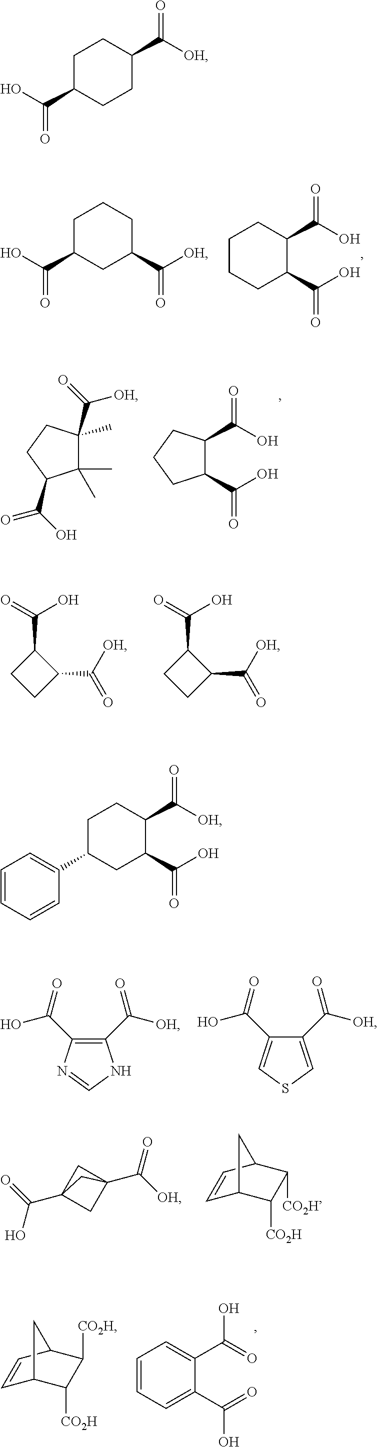

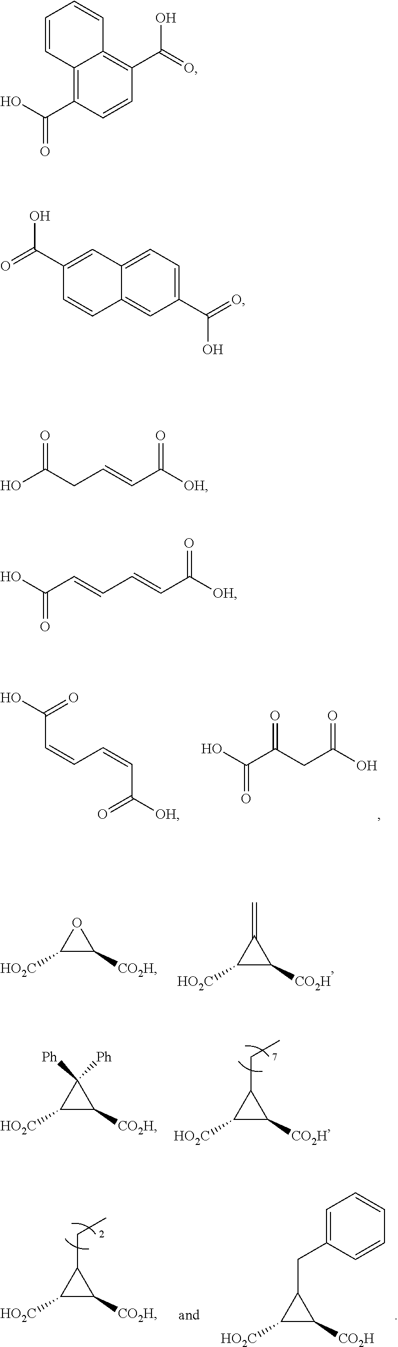

In some embodiments, dicarboxylic acid molecules, such as the ones described herein, may be further functionalized to contain one or more additional functional groups. Dicarboxylic acids may be further functionalized, for example, to provide an attachment point to an Fc domain monomer, an Fc domain, an Fc-binding peptide, an albumin protein, or an albumin protein-binding peptide (e.g., by way of a linker, such as a PEG linker).

In some embodiments, when the RSV F protein inhibitor is attached to Fc domain monomer, an Fc domain, an Fc-binding peptide, an albumin protein, or an albumin protein-binding peptide, the linking group may include a moiety including a carboxylic acid moiety and an amino moiety that are spaced by from 1 to 25 atoms. Examples of such linking groups include, but are not limited to,

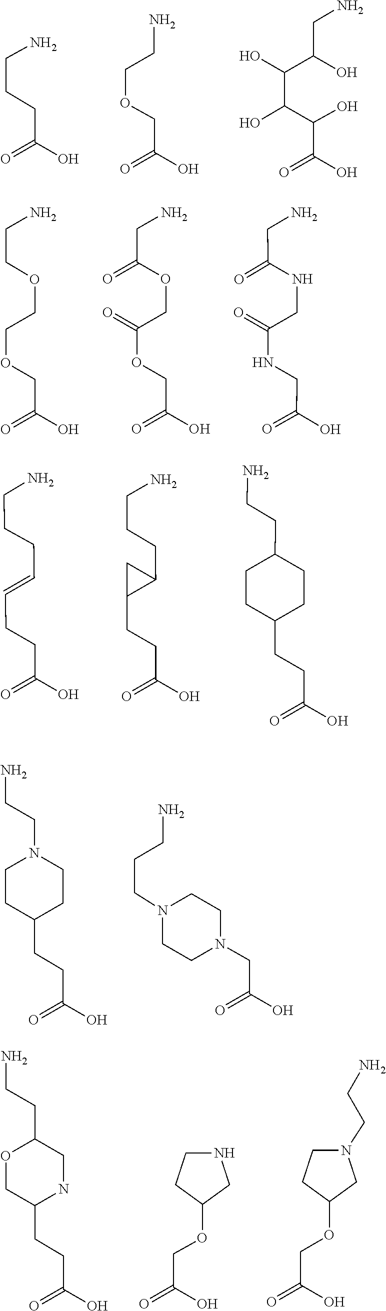

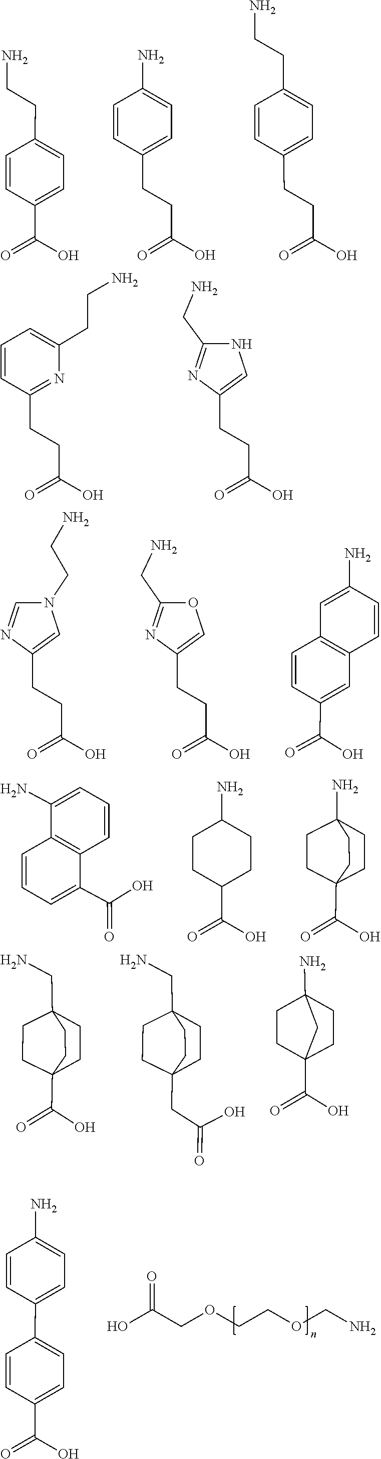

wherein n is an integer from 1 to 20 (e.g., n is 1, 2, 3, 4, 5, 6, 7, 8, 9, 10, 11, 12, 13, 14, 15, 16, 17, 18, 19, or 20).

In some embodiments, a linking group may including a moiety including a carboxylic acid moiety and an amino moiety, such as the ones described herein, may be further functionalized to contain one or more additional functional groups. Such linking groups may be further functionalized, for example, to provide an attachment point to an Fc domain monomer, an Fc domain, an Fc-binding peptide, an albumin protein, or an albumin protein-binding peptide (e.g., by way of a linker, such as a PEG linker).

In some embodiments, when the RSV F protein inhibitor is attached to Fc domain monomer, an Fc domain, an Fc-binding peptide, an albumin protein, or an albumin protein-binding peptide, the linking group may include a moiety including two or amino moieties (e.g., a diamino moiety) that are spaced by from 1 to 25 atoms. Examples of such linking groups include, but are not limited to,

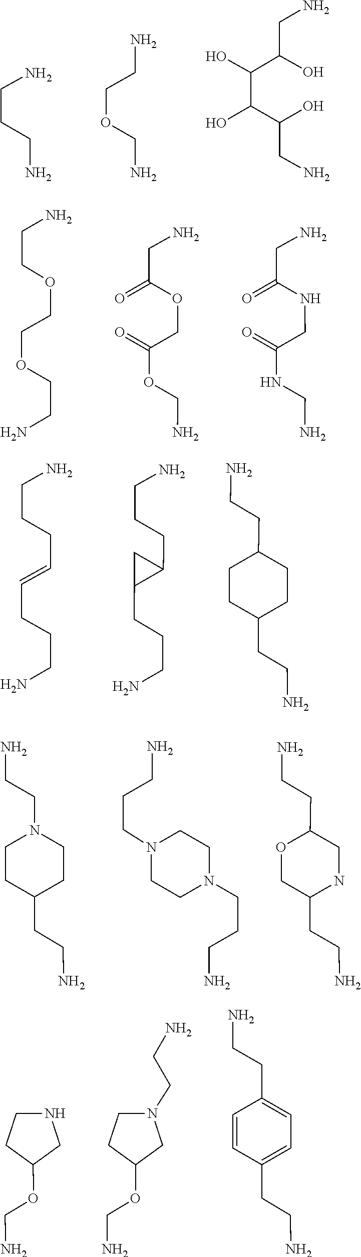

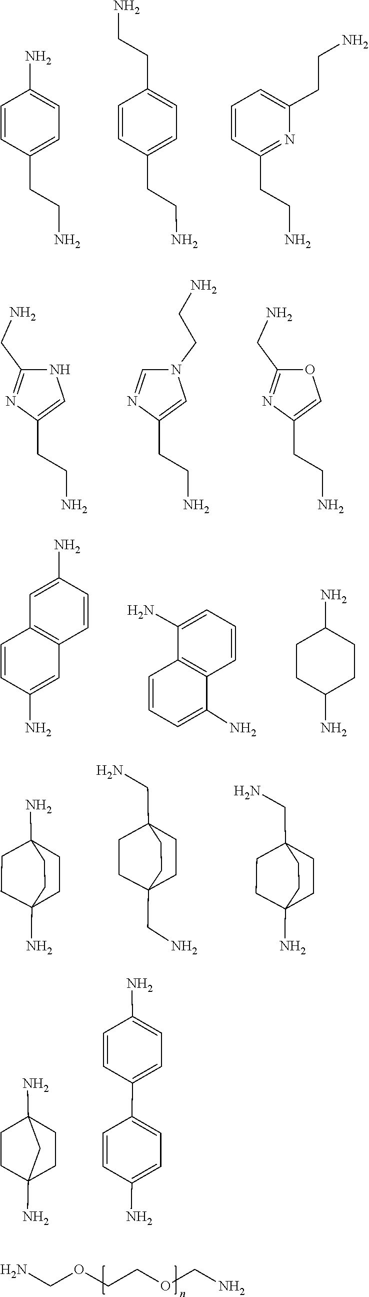

wherein n is an integer from 1 to 20 (e.g., n is 1, 2, 3, 4, 5, 6, 7, 8, 9, 10, 11, 12, 13, 14, 15, 16, 17, 18, 19, or 20).

In some embodiments, a linking group may including a diamino moiety, such as the ones described herein, may be further functionalized to contain one or more additional functional groups. Such diamino linking groups may be further functionalized, for example, to provide an attachment point to an Fc domain monomer, an Fc domain, an Fc-binding peptide, an albumin protein, or an albumin protein-binding peptide (e.g., by way of a linker, such as a PEG linker).

In some embodiments, a molecule containing an azide group may be used to form a linker, in which the azide group may undergo cycloaddition with an alkyne to form a 1,2,3-triazole linkage. In some embodiments, a molecule containing an alkyne group may be used to form a linker, in which the alkyne group may undergo cycloaddition with an azide to form a 1,2,3-triazole linkage. In some embodiments, a molecule containing a maleimide group may be used to form a linker, in which the maleimide group may react with a cysteine to form a C—S linkage. In some embodiments, a molecule containing one or more haloalkyl groups may be used to form a linker, in which the haloalkyl group may form a covalent linkage, e.g., C—N and C—O linkages, with a RSV F protein inhibitor.

In some embodiments, a linker (L or L′) may include a synthetic group derived from, e.g., a synthetic polymer (e.g., a polyethylene glycol (PEG) polymer). In some embodiments, a linker may include one or more amino acid residues. In some embodiments, a linker may be an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker (L or L′) may include one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

### Conjugation Chemistries

RSV F protein inhibitor monomers or dimers (e.g., in a conjugate of any one of formulas (1), (2), (D-I)-(D-IV), or (M-I)-(M-IV)) may be conjugated to an Fc domain monomer, an Fc domain, an Fc-binding peptide, an albumin protein, or an albumin protein-binding peptide, e.g., by way of a linker, by any standard conjugation chemistries known to those of skill in the art. The following conjugation chemistries are specifically contemplated, e.g., for conjugation of a PEG linker (e.g., a functionalized PEG linker) to an Fc domain monomer, an Fc domain, an Fc-binding peptide, an albumin protein, or an albumin protein-binding peptide.

Covalent conjugation of two or more components in a conjugate using a linker may be accomplished using well-known organic chemical synthesis techniques and methods. Complementary functional groups on two components may react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine. Site-specific conjugation to a polypeptide (e.g., an Fc domain monomer, and Fc domain, an Fc-binding peptide, an albumin protein, or an albumin protein-binding peptide) may accomplished using techniques known in the art. Exemplary techniques for site-specific conjugation of a small molecule to an Fc domain are provided in Agarwall. P., et al. Bioconjugate Chem. 26:176-192 (2015).

Other examples of functional groups capable of reacting with amino groups include, e.g., alkylating and acylating agents. Representative alkylating agents include: (i) an α-haloacetyl group, e.g., XCH₂CO— (where X=Br, Cl, or I); (ii) a N-maleimide group, which may react with amino groups either through a Michael type reaction or through acylation by addition to the ring carbonyl group; (iii) an aryl halide, e.g., a nitrohaloaromatic group; (iv) an alkyl halide; (v) an aldehyde or ketone capable of Schiff's base formation with amino groups; (vi) an epoxide, e.g., an epichlorohydrin and a bisoxirane, which may react with amino, sulfhydryl, or phenolic hydroxyl groups; (vii) a chlorine-containing of s-triazine, which is reactive towards nucleophiles such as amino, sulfhydryl, and hydroxyl groups; (viii) an aziridine, which is reactive towards nucleophiles such as amino groups by ring opening; (ix) a squaric acid diethyl ester; and (x) an α-haloalkyl ether.

Examples of amino-reactive acylating groups include, e.g., (i) an isocyanate and an isothiocyanate; (ii) a sulfonyl chloride; (iii) an acid halide; (iv) an active ester, e.g., a nitrophenylester or N-hydroxysuccinimidyl ester; (v) an acid anhydride, e.g., a mixed, symmetrical, or N-carboxyanhydride; (vi) an acylazide; and (vii) an imidoester. Aldehydes and ketones may be reacted with amines to form Schiff's bases, which may be stabilized through reductive amination.

It will be appreciated that certain functional groups may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

In some embodiments, a linker of the invention (e.g., L or L′, such as L^{C} of D-L-I), is conjugated (e.g., by any of the methods described herein) to E (e.g., an Fc domain or albumin protein). In preferred embodiments of the invention, the linker is conjugated by way of: (a) a thiourea linkage (i.e., —NH(C═S)NH—) to a lysine of E; (b) a carbamate linkage (i.e., —NH(C═O)—O) to a lysine of E; (c) an amine linkage by reductive amination (i.e., —NHCH₂) between a lysine and E; (d) an amide (i.e., —NH—(C═O)CH₂) to a lysine of E; (e) a cysteine-maleimide conjugate between a maleimide of the linker to a cysteine of E; (f) an amine linkage by reductive amination (i.e., —NHCH₂) between the linker and a carbohydrate of E (e.g., a glycosyl group of an Fc domain monomer or an Fc domain); (g) a rebridged cysteine conjugate, wherein the linker is conjugated to two cysteines of E; (h) an oxime linkage between the linker and a carbohydrate of E (e.g., a glycosyl group of an Fc domain monomer or an Fc domain); (i) an oxime linkage between the linker and an amino acid residue of E; (j) an azido linkage between the linker and E; (k) direct acylation of a linker to E; or (I) a thioether linkage between the linker and E.

In some embodiments, a linker is conjugated to E, wherein the linkage includes the structure —NH(C═NH)X—, wherein X is O, HN, or a bond. In some embodiments, a linker is conjugated to E, wherein the linkage between the remainder of the linker and E includes the structure —NH(C═O)NH—.

In some embodiments, a linker is conjugated to E, wherein the linkage includes the structure —R₉OR₉C(═O)NH—, wherein Rs is H, optionally substituted C₁-C₂₀ alkyl, optionally substituted C₃-C₂₀ cycloalkyl, optionally substituted C₂-C₂₀ heterocycloalkyl; optionally substituted C₅-C₁₅ aryl, or optionally substituted C₂-C₁₅ heteroaryl. In some embodiments, the linker is conjugated to E, wherein the linkage between the remainder of the linker and E includes the structure —CH₂OCH₂C(═O)NH—.

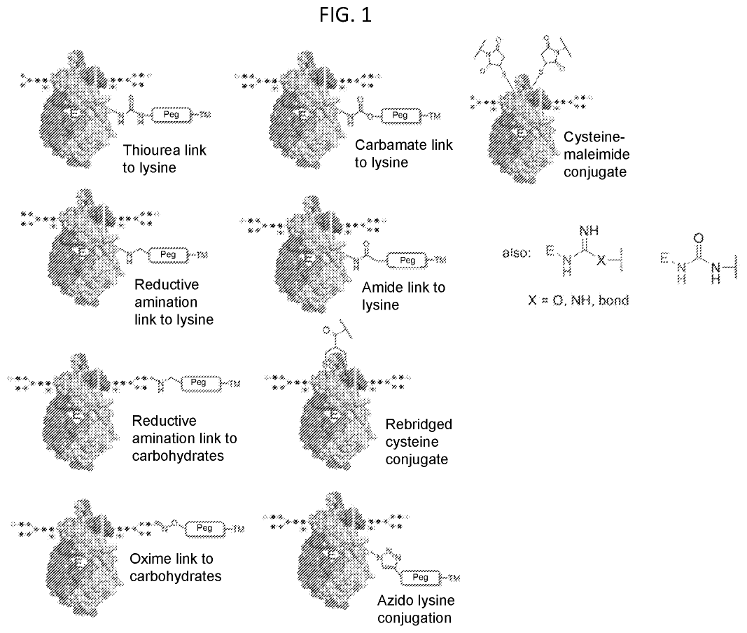

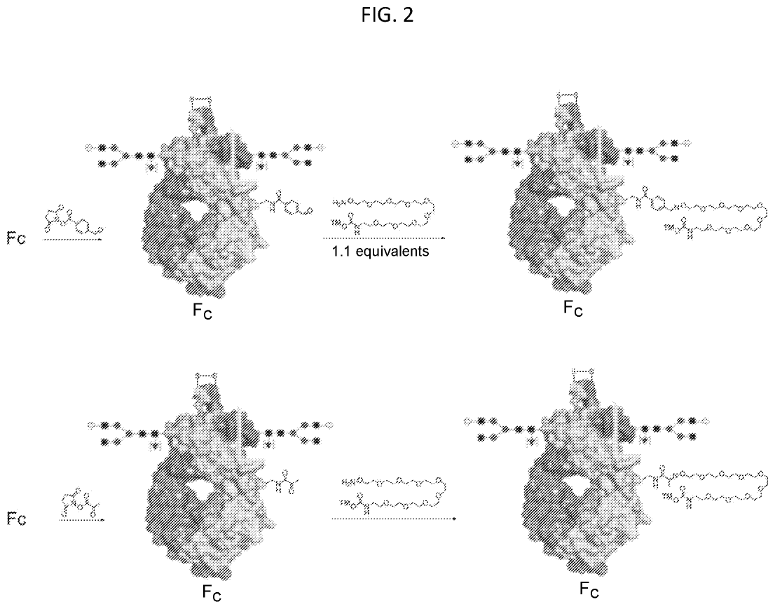

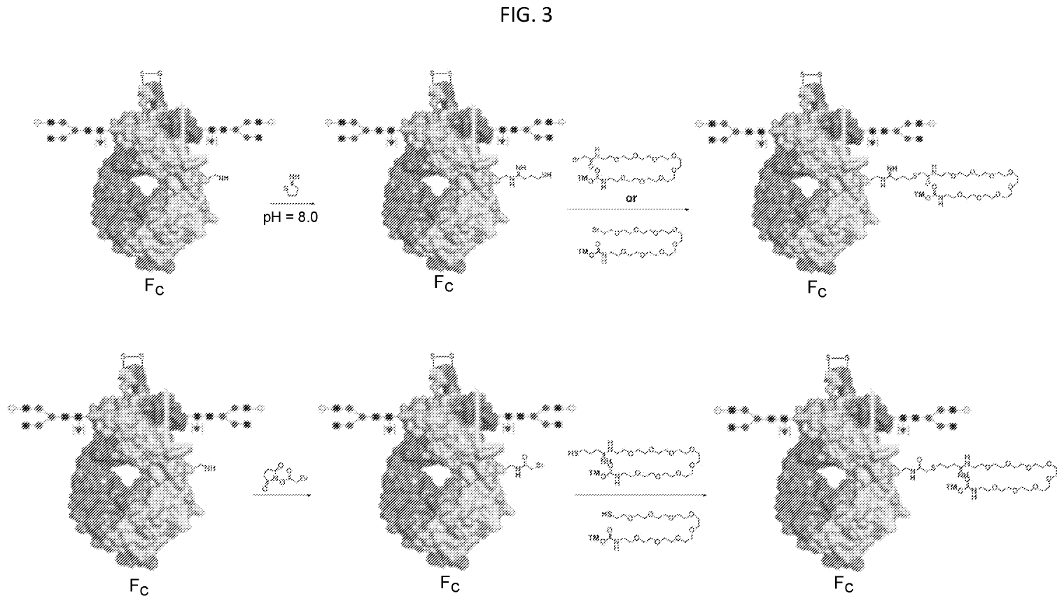

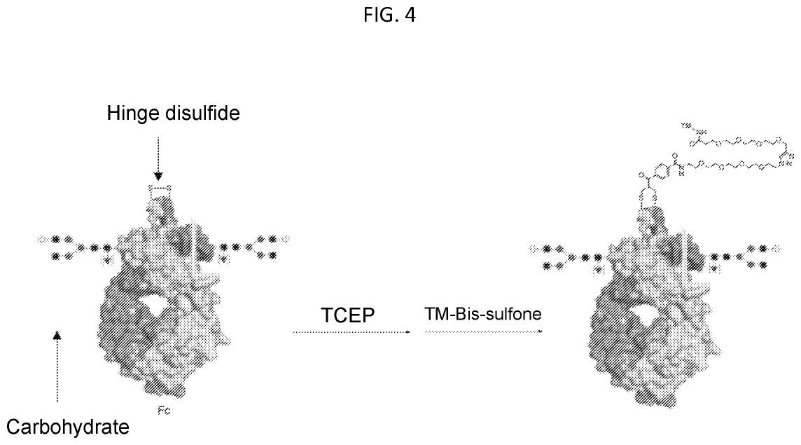

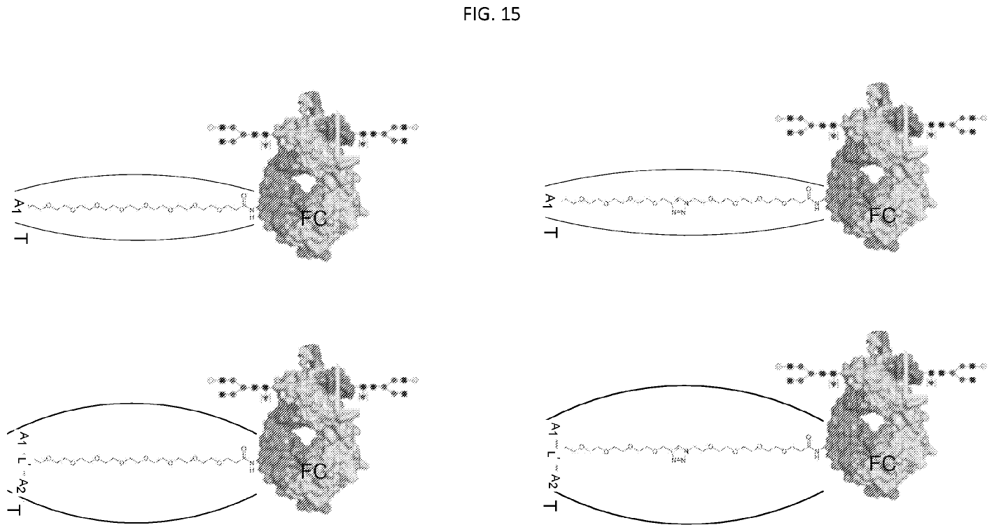

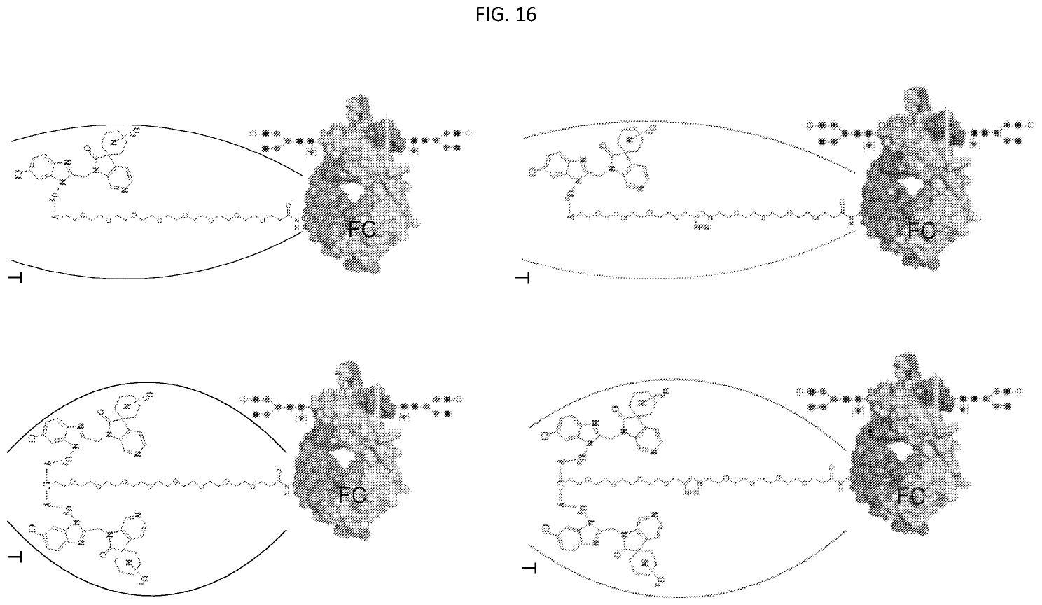

Exemplary linking strategies (e.g., methods for linking a monomer or a dimer of a RSV F protein inhibitor to E, such as, by way of a linker) are further depicted in FIGS. 1-4, 15, and 16.

In some embodiments, a linker (e.g., an active ester, e.g., a nitrophenylester or N-hydroxysuccinimidyl ester, or derivatives thereof (e.g., a functionalized PEG linker (e.g., azido-PEG₂-PEG₄₀-NHS ester), is conjugated to E, with a T of (e.g., DAR) of between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In these instances, the E-(PEG₂-PEG₄₀)-azide can react with an Int having a terminal alkyne linker (e.g., L, or L′, such as L^{C} of D-L-I) through click conjugation. During click conjugation, the copper-catalyzed reaction of the an azide (e.g., the Fc-(PEG₂-PEG₄₀)-azide) with the alkyne (e.g., the Int having a terminal alkyne linker (e.g., L or L′, such as L^{C} of D-L-I) forming a 5-membered heteroatom ring. In some embodiments, the linker conjugated to E is a terminal alkyne and is conjugated to an Int having a terminal azide. Exemplary preparations of preparations of E-(PEG₂-PEG₄₀)-azide are described in Examples 2, 3, and 12. One of skill in the art would readily understand the final product from a click chemistry conjugation.

Exemplary linking strategies (e.g., methods for linking a monomer or a dimer of a neuraminidase inhibitor to E, such as, by way of a linker) are further depicted in FIGS. 1-4, 15, and 16.

### VI. Combination Therapies

### Antiviral Agents

In some embodiments, one or more antiviral agents may be administered in combination (e.g., administered substantially simultaneously (e.g., in the same pharmaceutical composition or in separate pharmaceutical compositions, or administered separately at different times) with a conjugate described herein (e.g., a conjugate of any one of formulas (1), (2), (D-I)-(D-IV), or (M-I)-(M-IV)).

In some embodiments, the antiviral agent is an antiviral agent for the treatment of RSV. For example, the antiviral agent may be a viral replication inhibitor, a RSV F protein inhibitor, a polymerase inhibitor, or a fusion protein inhibitor. The antiviral agent may target either the virus or the host subject. The antiviral agent for the treatment of RSV used in combination with a conjugate described herein (e.g., a conjugate of any one of formulas (1), (2), (D-I)-(D-IV), or (M-I)-(M-IV)) may be selected from Presatovir, MDT 637, JNJ 179. In addition, derivatives of Presatovir, MDT 637, JNJ 179, such as those found in the literature, have RSV F protein inhibitor activity and are useful as RSV F protein inhibitors in combination with the compounds herein (see, for example, Cockerill et al. J. Med. Chem. 62(7): 3206-3227, 2018).

### Antiviral Vaccines

In some embodiments, any one of conjugates described herein (e.g., a conjugate of any one of formulas (1), (2), (D-I)-(D-IV), or (M-I)-(M-IV)) is administered in combination with an antiviral vaccine (e.g., a composition that elicits an immune response in a subject directed against a virus). The antiviral vaccine may be administered substantially simultaneously (e.g., in the same pharmaceutical composition or in separate pharmaceutical compositions) as the conjugates, or may be administered prior to or following the conjugates (e.g., within a period of 1 day, 2, days, 5, days, 1 week, 2 weeks, 3 weeks, 1 month, 2 months, 6 months, or 12 months, or more).

In some embodiments the viral vaccine includes an immunogen that elicits an immune response in the subject against RSV A or RSV B. In some embodiments the vaccine is administered as a nasal spray.

### VII. Methods

Methods described herein include, e.g., methods of protecting against or treating a viral infection (e.g., an RSV infection) in a subject and methods of preventing, stabilizing, or inhibiting the growth of viral particles. A method of treating a viral infection (e.g., an RSV infection) in a subject includes administering to the subject a conjugate described herein (e.g., a conjugate of any one of formulas (1), (2), (D-I)-(D-IV), or (M-I)-(M-IV)) or a pharmaceutical composition thereof. In some embodiments, the viral infection is cause by the respiratory syncytial virus (e.g., RSV A or RSV B). In some embodiments, the viral infection is caused by a resistant strain of virus. A method of preventing, stabilizing, or inhibiting the growth of viral particles or preventing the replication and spread of the virus includes contacting the virus or a site susceptible to viral growth with a conjugate described herein (e.g., a conjugate of any one of formulas (1), (2), (D-I)-(D-IV), or (M-I)-(M-IV)) or a pharmaceutical composition thereof.

Moreover, methods described herein also include methods of protecting against or treating viral infection in a subject by administering to the subject a conjugate described herein (e.g., a conjugate of any one of formulas (1), (2), (D-I)-(D-IV), or (M-I)-(M-IV)). In some embodiments, the method further includes administering to the subject an antiviral agent or an antiviral vaccine.

Methods described herein also include methods of protecting against or treating a viral infection in a subject by administering to said subject (1) a conjugate described herein (e.g., a conjugate of any one of formulas (1), (2), (D-I)-(D-IV), or (M-I)-(M-IV)) and (2) an antiviral agent or an antiviral vaccine. Methods described herein also include methods of preventing, stabilizing, or inhibiting the growth of viral particles or preventing the replication or spread of a virus, by contacting the virus or a site susceptible to viral growth with (1) a conjugate described herein (e.g., a conjugate of any one of formulas (1), (2), (D-I)-(D-IV), or (M-I)-(M-IV)) and (2) an antiviral agent or an antiviral vaccine.

In some embodiments, the conjugate described herein (e.g., a conjugate of any one of formulas (1), (2), (D-I)-(D-IV), or (M-I)-(M-IV)) is administered first, followed by administering of the antiviral agent or antiviral vaccine alone. In some embodiments, the antiviral agent or antiviral vaccine is administered first, followed by administering of the conjugate described herein alone. In some embodiments, the conjugate described herein and the antiviral agent or antiviral vaccine are administered substantially simultaneously (e.g., in the same pharmaceutical composition or in separate pharmaceutical compositions). In some embodiments, the conjugate described herein or the antiviral agent or antiviral vaccine is administered first, followed by administering of the conjugate described herein and the antiviral agent or antiviral vaccine substantially simultaneously (e.g., in the same pharmaceutical composition or in separate pharmaceutical compositions). In some embodiments, the conjugate described herein and the antiviral agent or antiviral vaccine are administered first substantially simultaneously (e.g., in the same pharmaceutical composition or in separate pharmaceutical compositions), followed by administering of the conjugate described herein or the antiviral agent or antiviral vaccine alone. In some embodiments, when a conjugate described herein (e.g., a conjugate of any one of formulas (1), (2), (D-I)-(D-IV), or (M-I)-(M-IV)) and an antiviral agent or antiviral vaccine are administered together (e.g., substantially simultaneously in the same or separate pharmaceutical compositions, or separately in the same treatment regimen), inhibition of viral replication of each of the conjugate and the antiviral agent or antiviral vaccine may be greater (e.g., occur at a lower concentration) than inhibition of viral replication of each of the conjugate and the antiviral agent or antiviral vaccine when each is used alone in a treatment regimen.

### VIII. Pharmaceutical Compositions and Preparations

A conjugate described herein may be formulated in a pharmaceutical composition for use in the methods described herein. In some embodiments, a conjugate described herein may be formulated in a pharmaceutical composition alone. In some embodiments, a conjugate described herein may be formulated in combination with an antiviral agent or antiviral vaccine in a pharmaceutical composition. In some embodiments, the pharmaceutical composition includes a conjugate described herein (e.g., a conjugate described by any one of formulas (1), (2), (D-I)-(D-IV), or (M-I)-(M-IV)) and pharmaceutically acceptable carriers and excipients.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acid residues such as glycine, glutamine, histidine, and lysine, and carbohydrates such as glucose, mannose, sucrose, and sorbitol.

Examples of other excipients include, but are not limited to, antiadherents, binders, coatings, compression aids, disintegrants, dyes, emollients, emulsifiers, fillers (diluents), film formers or coatings, flavors, fragrances, glidants (flow enhancers), lubricants, sorbents, suspending or dispersing agents, or sweeteners. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The conjugates herein may have ionizable groups so as to be capable of preparation as pharmaceutically acceptable salts. These salts may be acid addition salts involving inorganic or organic acids or the salts may, in the case of acidic forms of the conjugates herein be prepared from inorganic or organic bases. Frequently, the conjugates are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include, but are not limited to, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include, but are not limited to, sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Depending on the route of administration and the dosage, a conjugate herein or a pharmaceutical composition thereof used in the methods described herein will be formulated into suitable pharmaceutical compositions to permit facile delivery. A conjugate (e.g., a conjugate of any one of formulas (1), (2), (D-I)-(D-IV), or (M-I)-(M-IV)) or a pharmaceutical composition thereof may be formulated to be administered intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravascularly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). Depending on the route of administration, a conjugate herein or a pharmaceutical composition thereof may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

A conjugate described herein may be formulated in a variety of ways that are known in the art. For use as treatment of human and animal subjects, a conjugate described herein can be formulated as pharmaceutical or veterinary compositions. Depending on the subject (e.g., a human) to be treated, the mode of administration, and the type of treatment desired, e.g., prophylaxis or therapy, a conjugate described herein is formulated in ways consonant with these parameters. A summary of such techniques is found in Remington: The Science and Practice of Pharmacy, 22nd Edition, Lippincott Williams & Wilkins (2012); and Encyclopedia of Pharmaceutical Technology, 4th Edition, J. Swarbrick and J. C. Boylan, Marcel Dekker, New York (2013), each of which is incorporated herein by reference.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, and preservatives. The conjugates can be administered also in liposomal compositions or as microemulsions. Systemic administration may also include relatively noninvasive methods such as the use of suppositories, transdermal patches, transmucosal delivery and intranasal administration. Oral administration is also suitable for conjugates herein. Suitable forms include syrups, capsules, and tablets, as is understood in the art.

The pharmaceutical compositions can be administered parenterally in the form of an injectable formulation. Pharmaceutical compositions for injection can be formulated using a sterile solution or any pharmaceutically acceptable liquid as a vehicle. Formulations may be prepared as solid forms suitable for solution or suspension in liquid prior to injection or as emulsions. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Such injectable compositions may also contain amounts of nontoxic auxiliary substances such as wetting or emulsifying agents, pH buffering agents, such as sodium acetate and sorbitan monolaurate. Formulation methods are known in the art, see e.g., Pharmaceutical Preformulation and Formulation, 2nd Edition, M. Gibson, Taylor & Francis Group, CRC Press (2009).

The pharmaceutical compositions can be prepared in the form of an oral formulation. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Other pharmaceutically acceptable excipients for oral formulations include, but are not limited to, colorants, flavoring agents, plasticizers, humectants, and buffering agents. Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release of a conjugate described herein (e.g., a conjugate of any one of formulas (1), (2), (D-I)-(D-IV), or (M-I)-(M-IV)) or a pharmaceutical composition thereof can be achieved by appropriate coating of a tablet, capsule, pellet, or granulate formulation of the conjugate, or by incorporating the conjugate into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The pharmaceutical composition may be formed in a unit dose form as needed. The amount of active component, e.g., a conjugate described herein (e.g., a conjugate of any one of formulas (1), (2), (D-I)-(D-IV), or (M-I)-(M-IV)), included in the pharmaceutical compositions are such that a suitable dose within the designated range is provided (e.g., a dose within the range of 0.01-100 mg/kg of body weight).

### IX. Routes of Administration and Dosages

In any of the methods described herein, conjugates herein may be administered by any appropriate route for treating or protecting against a viral infection (e.g., an RSV infection), or for preventing, stabilizing, or inhibiting the proliferation or spread of a virus (e.g., an RSV virus). Conjugates described herein may be administered to humans, domestic pets, livestock, or other animals with a pharmaceutically acceptable diluent, carrier, or excipient. In some embodiments, administering includes administration of any of the conjugates described herein (e.g., conjugates of any one of formulas (1), (2), (D-I)-(D-IV), or (M-I)-(M-IV)) or compositions intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravascularly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). In some embodiments, if an antiviral agent is also administered in addition to a conjugate described herein, the antiviral agent or a pharmaceutical composition thereof may also be administered in any of the routes of administration described herein.

The dosage of a conjugate described herein (e.g., a conjugate of any one of formulas (1), (2), (D-I)-(D-IV), or (M-I)-(M-IV)) or pharmaceutical compositions thereof depends on factors including the route of administration, the disease to be treated (e.g., the extent and/or condition of the viral infection), and physical characteristics, e.g., age, weight, general health, of the subject. Typically, the amount of the conjugate or the pharmaceutical composition thereof contained within a single dose may be an amount that effectively prevents, delays, or treats the viral infection without inducing significant toxicity. A pharmaceutical composition may include a dosage of a conjugate described herein ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 1 to about 30 mg/kg. In some embodiments, when a conjugate described herein (e.g., a conjugate of any one of formulas (1), (2), (D-I)-(D-IV), or (M-I)-(M-IV)) and an antiviral agent or antiviral vaccine are administered in combination (e.g., substantially simultaneously in the same or separate pharmaceutical compositions, or separately in the same treatment regimen), the dosage needed of the conjugate described herein may be lower than the dosage needed of the conjugate if the conjugate was used alone in a treatment regimen.

A conjugate described herein (e.g., a conjugate of any one of formulas (1), (2), (D-I)-(D-IV), or (M-I)-(M-IV)) or a pharmaceutical composition thereof may be administered to a subject in need thereof, for example, one or more times (e.g., 1-10 times or more; 1, 2, 3, 4, 5, 6, 7, 8, 9, or 10 times) daily, weekly, monthly, biannually, annually, or as medically necessary. Dosages may be provided in either a single or multiple dosage regimens. The timing between administrations may decrease as the medical condition improves or increase as the health of the patient declines. The dosage and frequency of administration may be adapted by the physician in accordance with conventional factors such as the extent of the infection and different parameters of the subject.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Preparation of Fc Constructs

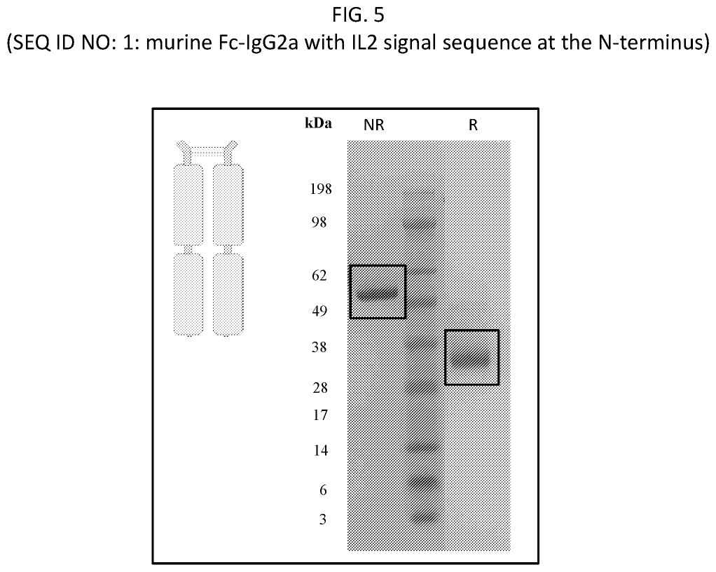

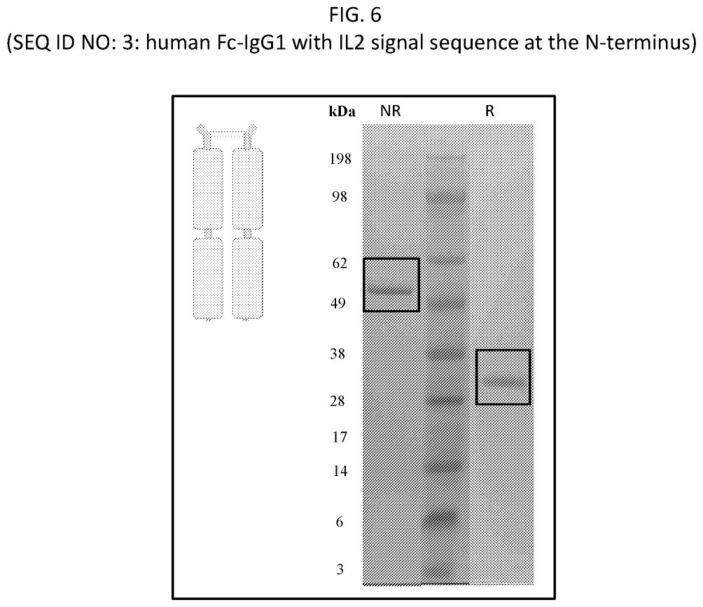

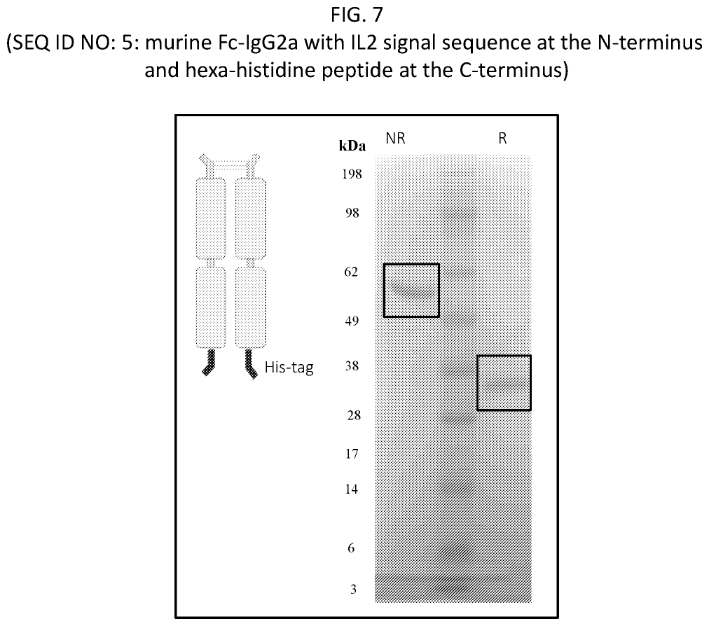

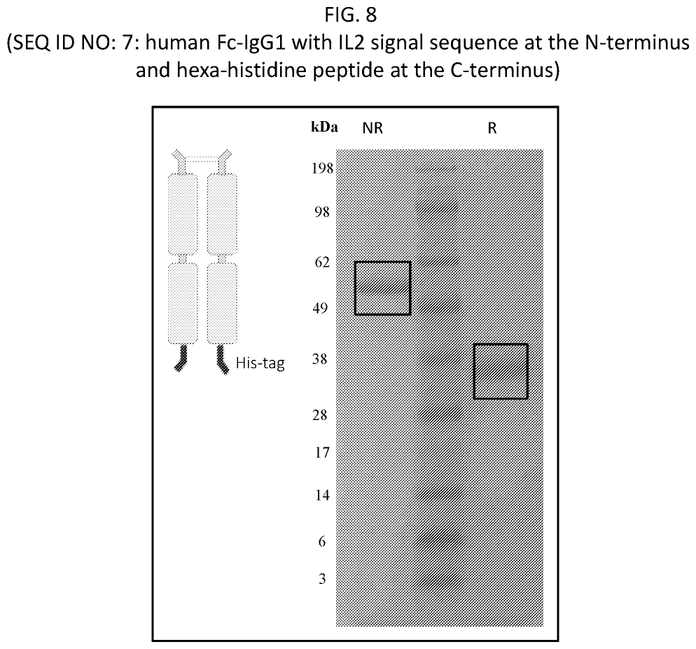

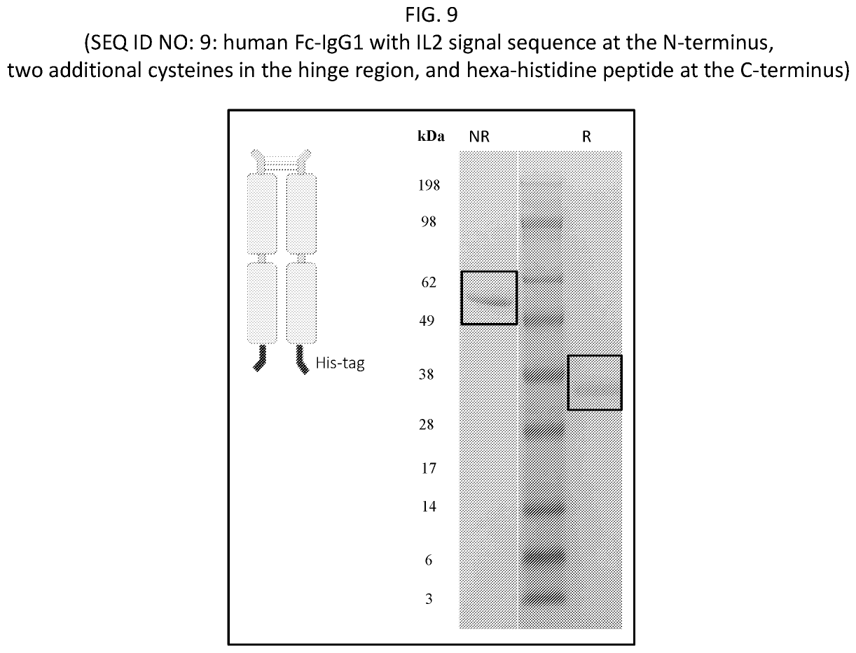

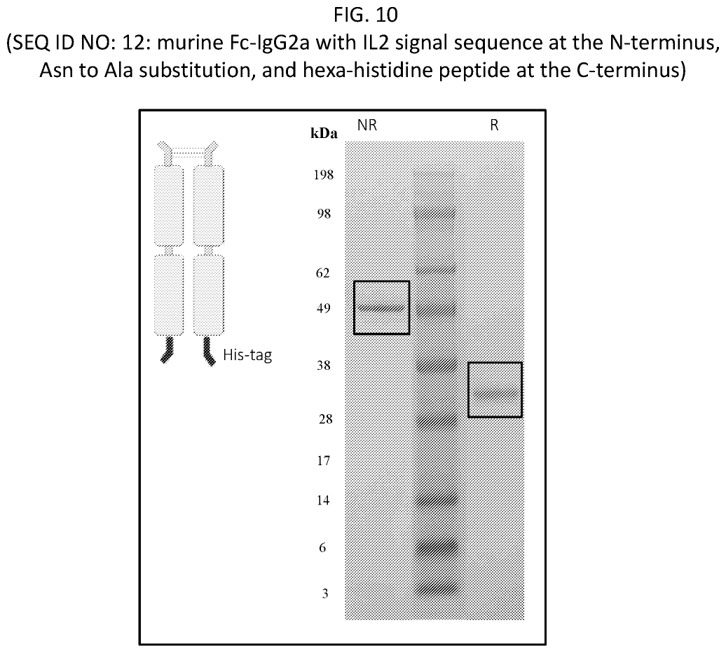

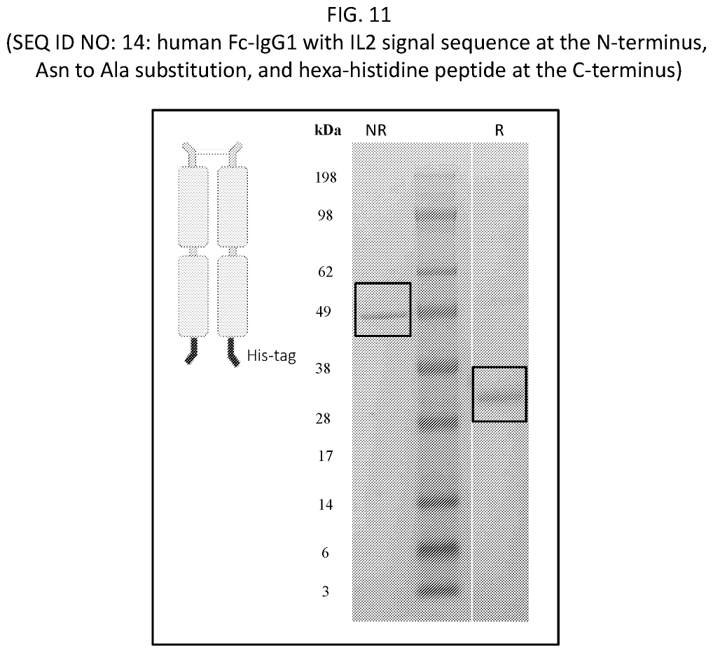

Reverse translations of the amino acids including the protein constructs (SEQ ID NOs: 1, 3, 5, 7, 9, 12, and 14) were synthesized by solid-phase synthesis. The oligonucleotide templates were cloned into pcDNA3.1 (Life Technologies, Carlsbad, Calif., USA) at the cloning sites BamHI and XhoI (New England Biolabs, Ipswich, Mass., USA) and included signal sequences derived from the human Interleukin-2 or human albumin. The pcDNA3.1 plasmids were transformed into Top10 E. coli cells (LifeTech). DNA was amplified, extracted, and purified using the PURELINK® HiPure Plasmid Filter Maxiprep Kit (LifeTech). The plasmid DNA is delivered, using the EXPIFECTAMINE™ 293 Transfection Kit (LifeTech), into HEK-293 cells per the manufacturer's protocol. Cells were centrifuged, filtered, and the supernatants were purified using MabSelect Sure Resin (GE Healthcare, Chicago, Ill., USA). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards. Reduced and non-reduced lanes are denoted by “R” and “NR”. FIGS. 5-11 show non-reducing and reducing SDS-PAGE of an Fc domain formed from Fc domain monomers having the sequences of SEQ ID NOs: 1, 3, 5, 7, 9, 12, and 14, respectively.

### Example 2. General Procedure for Synthesis of Azido Fc

Preparation of PEG4-azido NHS ester solution (0.050 M) in DMF/PBS: 91.85 mg of PEG4-azido NHS ester was dissolved in 0.500 mL of DMF at 0° C. and diluted to 4.635 mL by adding PBS 1× buffer at 0° C. This solution was used for preparing other PEG4-azido Fc with variety of DAR values by adjusting the equivalents of this PEG4-azido NHS ester PBS solution.

Preparation of PEG4-azido Fc: 0.050M PEG4-azidoNHS ester PBS buffer solution (1.803 mL, 90.13 μmol, 9.5 equivalents) was added to a solution of h-IgG1 Fc (SEQ ID NO: 4) (552.2 mg in 33.75 mL of pH 7.4 PBS, MW-58,200 Da, 9.487 μmol) and the mixture was shaken gently for 2 hours at ambient temperature. The solution was concentrated by using six centrifugal concentrators (30,000 MWCO, 15 mL) to a volume of ˜1.5 mL. The crude mixture was diluted 1:10 in PBS pH 7.4, and concentrated again. This wash procedure was repeated for total of three times. The concentrated Fc-PEG4-azide was diluted to 33.75 mL with pH 7.4 PBS 1× buffer and ready for Click conjugation. The purified material was quantified using a NANODROP™ UV visible spectrophotometer (using a calculated extinction coefficient based on the amino acid sequence of h-IgG1 (SEQ ID NO: 4). Yield is quantitative after purification.

Preparation of 0.05M azido-acetate NHS ester solution in DMF/PBS: 50.01 mg of 2-azidoacetic acid NHS ester was dissolved in DMF (2.445 mL) at 0° C. and diluted with PBS (4.946 mL) buffer at 0° C. This solution was used for preparing other azido Fc's with variety of DAR values by adjusting the equivalents of this azido acetate NHS ester solution.

Preparation of azido-acetate Fc: 0.05M azido-acetate NHS ester PBS buffer solution (4.723 mL, 236.1 μmol, 12.6 equivalents) was added to a solution of h-IgG1 Fc (552.2 mg in 47.92 mL of pH 7.4 PBS, MW-53360 Da, 18.74 μmol) then the mixture was shaken rotated for 2 hours at ambient temperature. The solution was concentrated by using six centrifugal concentrators (30,000 MWCO, 15 mL) to a volume of ˜1.5 mL. The crude mixture was diluted 1:10 in PBS pH 7.4, and concentrated again. This wash procedure was repeated for total of three times. The concentrated azido acetate Fc was diluted to 47.92 mL with pH 7.4 PBS buffer. This material was quantified using a NANODROP™ UV visible spectrophotometer using a calculated extinction coefficient based on the amino acid sequence of h-IgG1 (SEQ ID NO: 4). Yield was quantitative after purification.

### Example 3. Synthesis of Recombinant Mouse Serum Albumin (MSA)-PEG4-azide

PEG4-azidoNHS ester (98%, 81.7 μmol, 4.5 equivalents, 32.4 mg in 0.3 mL of DMF and diluted to 1.63 mL with pH 7.4 PBS 1× buffer solution) was added to a solution of recombinant mouse serum albumin (SEQ ID NO: 71) (1200 mg in 75.0 mL of pH 7.4 PBS, MW-66,000 Da, 18.2 μmol) and the mixture was shaken gently for 12 hours at ambient temperature. The solution was concentrated using a centrifugal concentrator (30,000 MWCO) to a volume of ˜1.5 mL. The crude mixture was diluted 1:10 in PBS pH 7.4, and concentrated again. This wash procedure was repeated for total of three times. The small molecule reagent was removed with this wash procedure. The concentrated MSA-PEG4-azide was diluted to 75.0 mL with pH 7.4 PBS 1× buffer and ready for Click conjugation. The purified material was quantified using a NANODROP™ UV visible spectrophotometer (using a calculated extinction coefficient based on the amino acid sequence of h-IgG1). Yield is quantitative after purification. DAR=3.5 determined by MALDI. The DAR value can be adjusted by altering the equivalents of PEG4-azido NHS ester similar to h-IgG1 Fc (Example 2).

### Example 4. Synthesis of Int-1

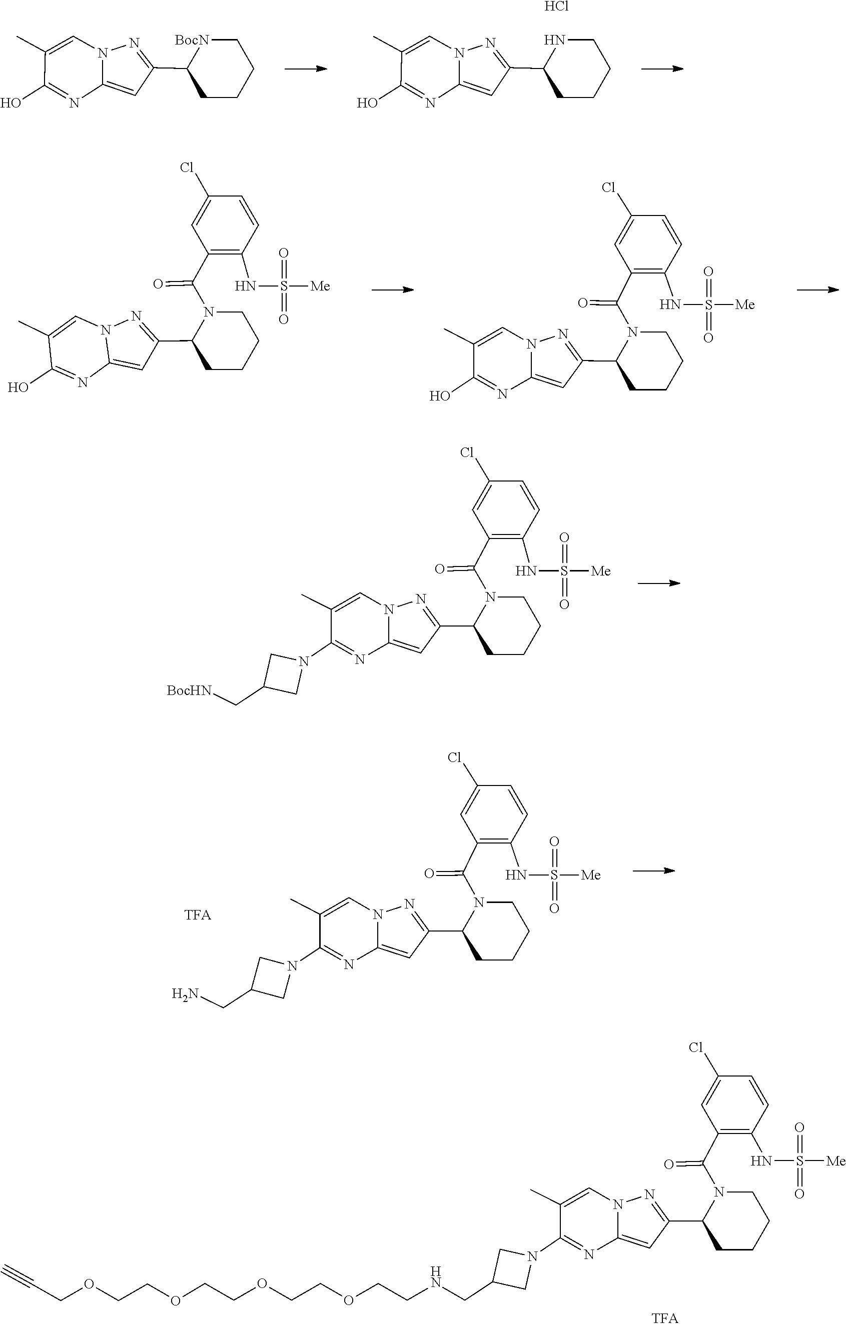

Step a.

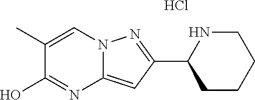 [SMILES: Cl ; Cc1cn2nc([C@@H]3CCCCN3)cc2nc1O]

To a solution of pyrazolopyrimidine intermediate (3.32 g, 10 mmol, described in PCT publication WO2011163518A1) in anhydrous THF (10 ml) was added 4N HCl solution in dioxane (10 ml). The reaction became gel-like. It was stirred at room temperature for 1 day, then concentrated by rotary evaporation. The residue was dissolved in MeOH (20 ml) and heated at 45° C. for 30 minutes to drive the reaction to completion. The mixture was then concentrated again and further dried under high vacuum. The crude product was carried to the subsequent step without further purification. Yield 2.7 g, quantitative yield. Ion found by LCMS: [M+H]⁺=233.

Step b.

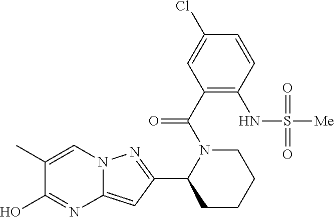 [SMILES: Cc1cn2nc([C@@H]3CCCCN3C(=O)c3cc(Cl)ccc3NS(C)(=O)=O)cc2nc1O]

To a solution of 5-chloro-2-methanesulfonamidobenzoic acid (499.4 mg, 2 mmol) and HATU (798.4 mg, 2.1 mmol) in anhydrous DMF (4 ml) was added DIPEA (520 mg, 4 mmol) and the step-a product (537.6 mg, 2 mmol). The reaction mixture was stirred for 30 minutes, then dripped into water (50 ml). The white product was filtered, washed with water, and further dried under high vacuum. The crude product was carried to the subsequent step without further purification. Yield 738.7 mg, 82.8%. Ion found by LCMS: [M+H]⁺=464.0.

Step c.

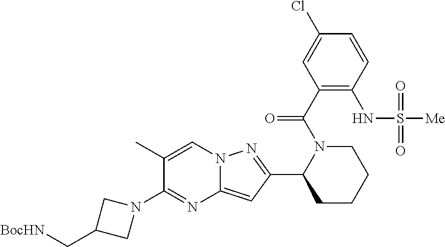 [SMILES: Cc1cn2nc([C@@H]3CCCCN3C(=O)c3cc(Cl)ccc3NS(C)(=O)=O)cc2nc1N1CC(CNC(=O)OC(C)(C)C)C1]

To a solution of the step-b product (104.6 mg, 0.226 mmol) in anhydrous DMF (1 ml), DIPEA (65 mg, 0.5 mmol) and BOP reagent (104.9 mg, 0.238 mmol) was added. The resulting mixture was heated at 70° C. for 10 minutes, then 3-Boc-aminomethylazetedine (63 mg, 0.27 mmol) was added. The reaction was continued at 70° C. for 1 hour. The product was purified by RPLC (25 to 75% acetonitrile and water, using 0.1% TFA as modifier). Yield 124.8 mg, 87.3%. Ion found by LCMS: [M+H]⁺=6332.2.

Step d.

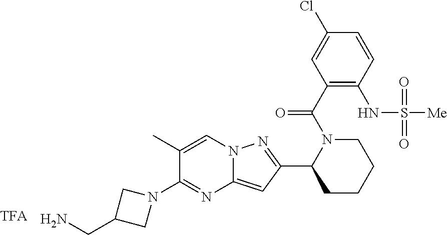 [SMILES: Cc1cn2nc([C@@H]3CCCCN3C(=O)c3cc(Cl)ccc3NS(C)(=O)=O)cc2nc1N1CC(CN)C1]

The step-c product in DCM (1.5 ml) was added TFA (0.5 ml). The mixture was stirred for 40 minutes, then concentrated and purified by RPLC (5% to 60% acetonitrile and water, using 0.1% TFA as modifier). Yield 113.2 mg, 89.3%. Ion found by LCMS: [M+H]⁺=532.2.

Step e.

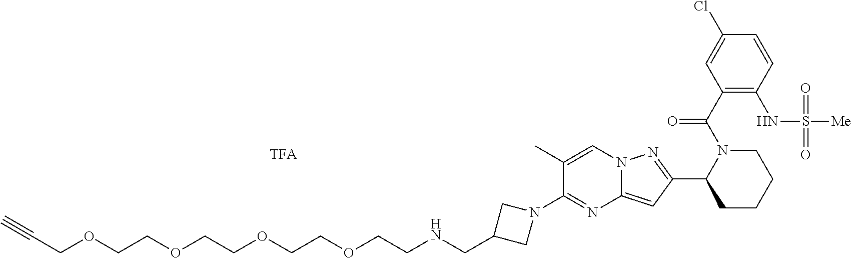 [SMILES: C#CCOCCOCCOCCOCCNCC1CN(c2nc3cc([C@@H]4CCCCN4C(=O)c4cc(Cl)ccc4NS(C)(=O)=O)nn3cc2C)C1]

To a solution of the step-d product (111.3 mg, 0.173 mmol) in anhydrous THF (1 ml) was added propargyl-PEG4-bromide (62 mg, 0.21 mmol) and sodium carbonate (20 mg, 0.19 mmol). The resulting mixture was heated at 50° C. overnight. It was then purified by HPLC (5% to 60% acetonitrile and water, using 0.1% TFA as modifier). Yield 23.3 mg, 15.7%. Ion found by LCMS: [M+H]⁺=746.2.

### Example 5. Synthesis of Int-2

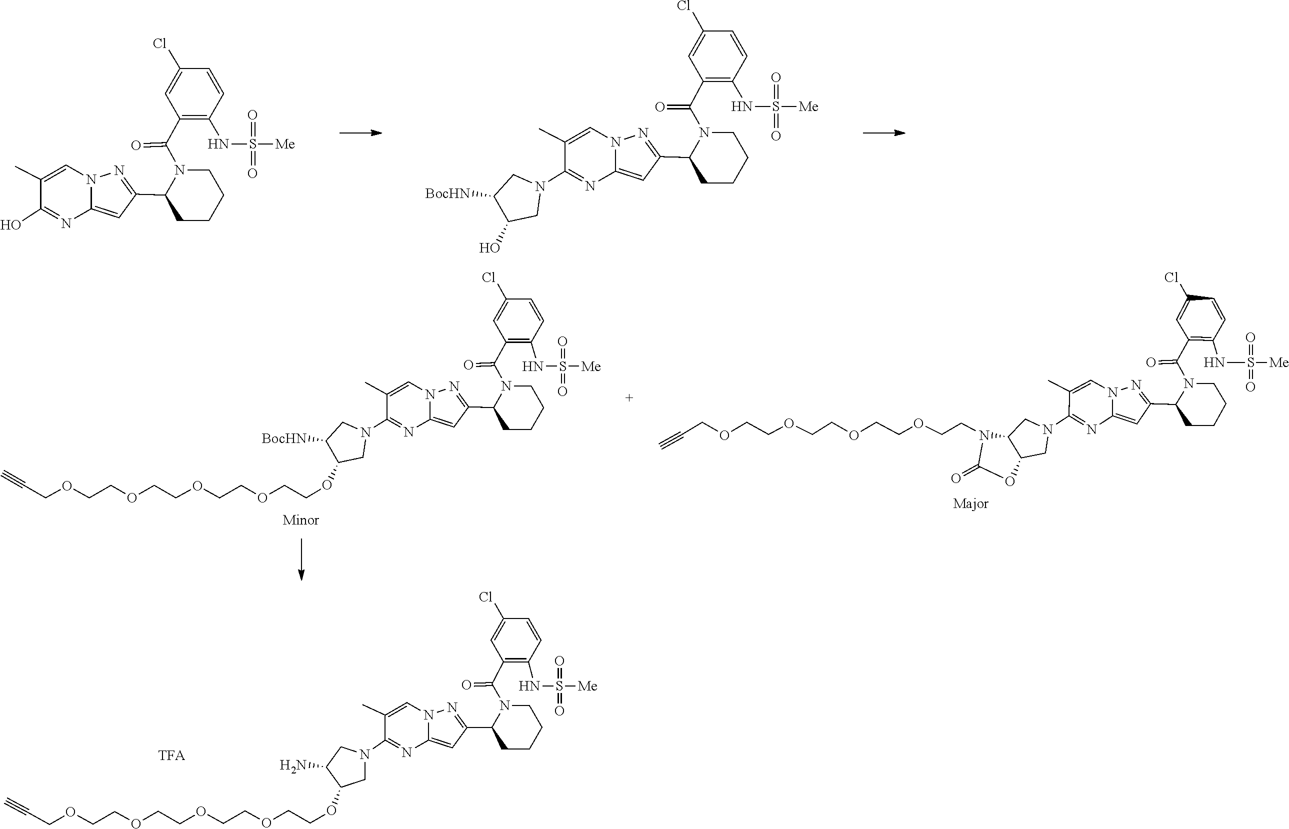

Step a.

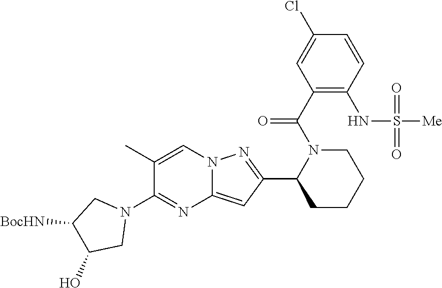 [SMILES: Cc1cn2nc([C@@H]3CCCCN3C(=O)c3cc(Cl)ccc3NS(C)(=O)=O)cc2nc1N1C[C@H](O)[C@H](NC(=O)OC(C)(C)C)C1]

To a solution of starting material (231.5 mg, 0.5 mmol, described in Example 4, Int-1) in anhydrous DMF (1 ml) was added DIPEA (129.2 mg, 1 mmol) and BOP reagent (243.3 mg, 0.55 mmol). The resulting mixture was heated at 70° C. for 10 minutes, then (3R,4S)-(4-hydroxy-pyrrolidin-3-yl)-carbamic acid tert-butyl ester (132 mg, 0.65 mmol) was added. The reaction was continued at 70° C. for 1 hour. It was purified by RPLC (25% to 65% acetonitrile and water, using 0.1% TFA as modifier). Yield 207.8 mg, 64.2%. Ion found by LCMS: [M+H]⁺=648.2.

Step b.

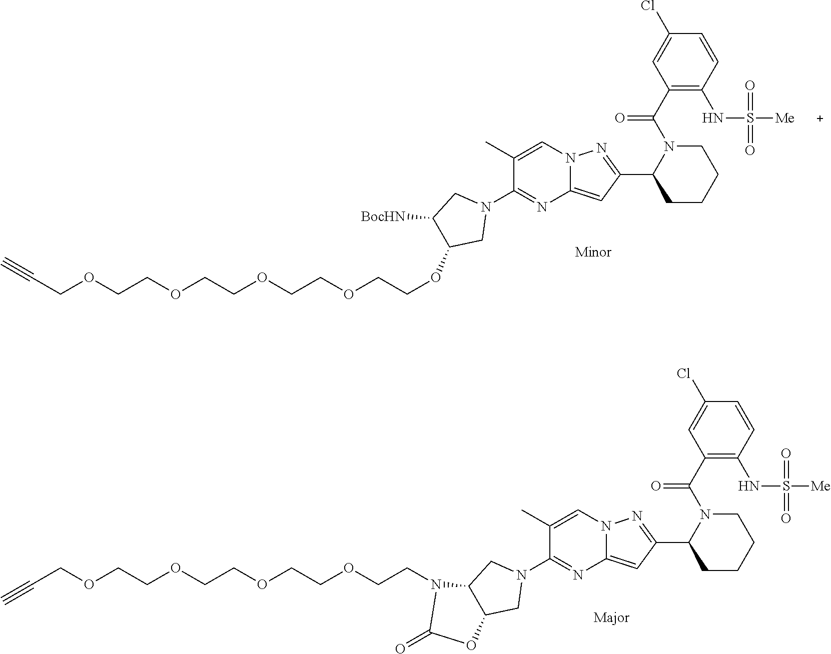 [SMILES: C#CCOCCOCCOCCOCCO[C@H]1CN(c2nc3cc([C@@H]4CCCCN4C(=O)c4cc(Cl)ccc4NS(C)(=O)=O)nn3cc2C)C[C@H]1NC(=O)OC(C)(C)C ; C#CCOCCOCCOCCOCCN1C(=O)O[C@H]2CN(c3nc4cc([C@@H]5CCCCN5C(=O)c5cc(Cl)ccc5NS(C)(=O)=O)nn4cc3C)C[C@H]21]

A flame-dried reaction flask was purged with nitrogen and charged with the step-a product (50 mg, 0.0772 mmol) and anhydrous DMF (1 ml). After the solution was cooled in an ice-water bath, it was treated with sodium hydride (60% suspension in oil, 12.5 mg, 0.31 mmol) followed by propargyl-PEG4-bromide (45.6 mg, 0.154 mmol). The ice-water bath was removed, and the reaction was stirred for 3 hours. It was then purified by RPLC (5% to 80% acetonitrile and water, using 0.1% TFA as modifier). Yield 14.2 mg of the minor desired product, 21.4% (Ion found by LCMS: [M+H]⁺=862.2) and 23.4 mg of the major side product, 38.5% (Ion found by LCMS: [M+H]⁺=788.2).

Step c.

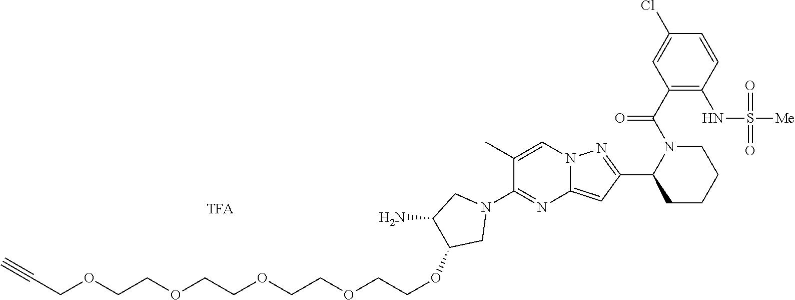 [SMILES: C#CCOCCOCCOCCOCCO[C@H]1CN(c2nc3cc([C@@H]4CCCCN4C(=O)c4cc(Cl)ccc4NS(C)(=O)=O)nn3cc2C)C[C@H]1N]

The minor product in step-b (14.2 mg, 0.0165 mmol) was dissolved in TFA (0.5 ml), and the solution was stirred for 20 minutes. It was then purified by HPLC (5 to 50% acetonitrile and water, using 0.1% TFA as modifier). Yield 11.9 mg, 82.3%. Ion found by LCMS: [M+H]⁺=762.2.

### Example 6. Synthesis of Int-3

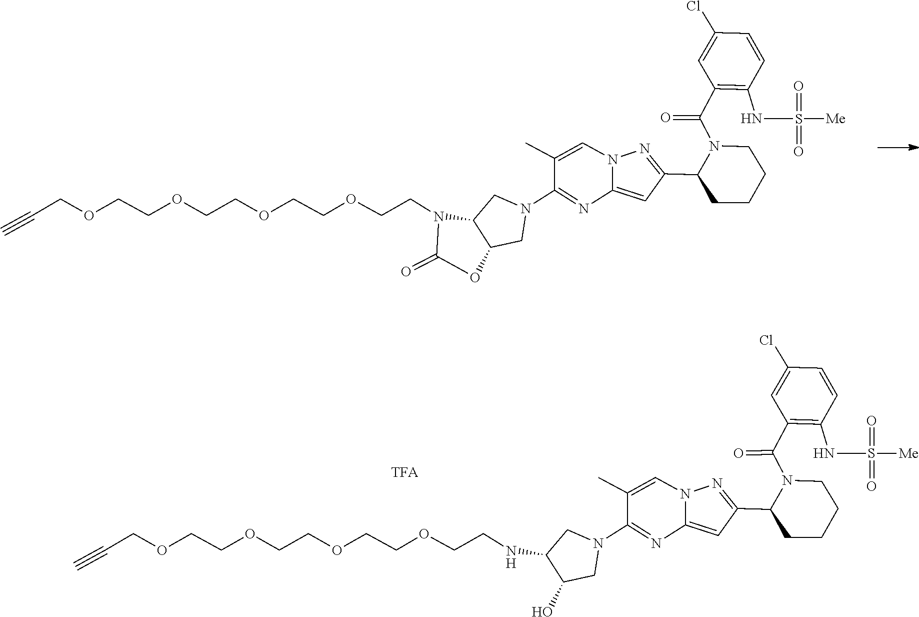 [SMILES: C#CCOCCOCCOCCOCCN1C(=O)O[C@H]2CN(c3nc4cc([C@@H]5CCCCN5C(=O)c5cc(Cl)ccc5NS(C)(=O)=O)nn4cc3C)C[C@H]21 ; C#CCOCCOCCOCCOCCN[C@@H]1CN(c2nc3cc([C@@H]4CCCCN4C(=O)c4cc(Cl)ccc4NS(C)(=O)=O)nn3cc2C)C[C@@H]1O]

The major product from step-b of Example 5 (Int-2) (23.4 mg, 0.0297 mmol) was dissolved in MeOH (2 ml), and the solution was treated with 30% NaOH in water (0.5 ml) and 1 ml of water. The resulting mixture was heated at 70° C. for 3 hours. It was then cooled to room temperature and acidified with 4N HCl solution in dioxane (0.2 ml). After concentrating by rotary evaporation, the reaction mixture was purified by preparative HPLC (5 to 50% acetonitrile and water, using 0.1% TFA as modifier). Yield 12.9 mg, 49.6%. Ion found by LCMS: [M+H]⁺=762.2.

### Example 7. Synthesis of Int-4

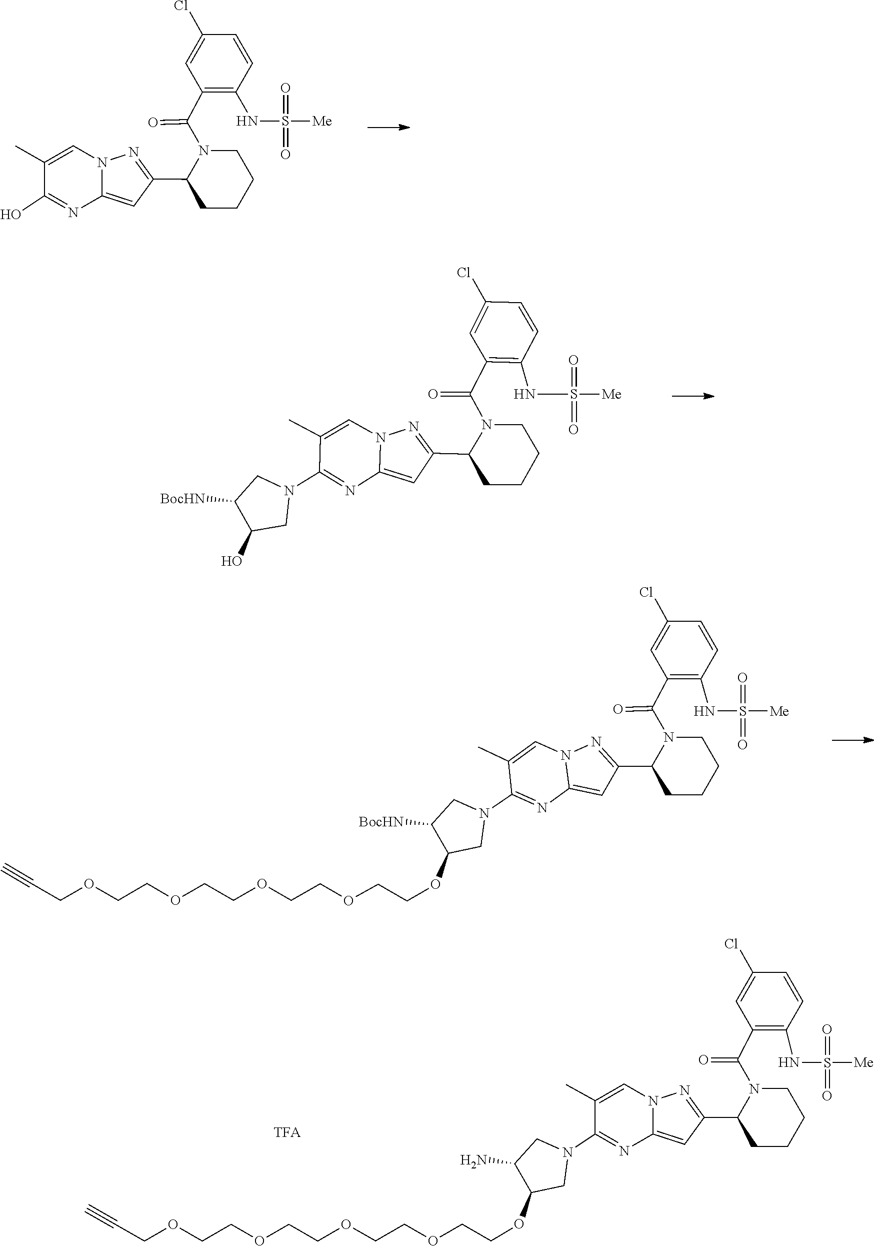 [SMILES: Cc1cn2nc([C@@H]3CCCCN3C(=O)c3cc(Cl)ccc3NS(C)(=O)=O)cc2nc1O ; Cc1cn2nc([C@@H]3CCCCN3C(=O)c3cc(Cl)ccc3NS(C)(=O)=O)cc2nc1N1C[C@@H](O)[C@H](NC(=O)OC(C)(C)C)C1 ; C#CCOCCOCCOCCOCCO[C@@H]1CN(c2nc3cc([C@@H]4CCCCN4C(=O)c4cc(Cl)ccc4NS(C)(=O)=O)nn3cc2C)C[C@H]1NC(=O)OC(C)(C)C ; C#CCOCCOCCOCCOCCO[C@@H]1CN(c2nc3cc([C@@H]4CCCCN4C(=O)c4cc(Cl)ccc4NS(C)(=O)=O)nn3cc2C)C[C@H]1N]

Step a.

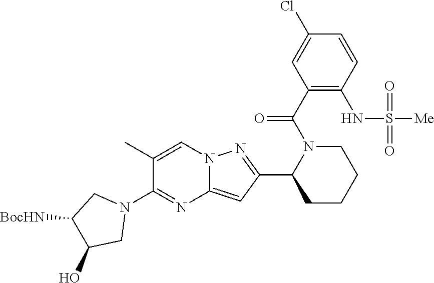 [SMILES: Cc1cn2nc([C@@H]3CCCCN3C(=O)c3cc(Cl)ccc3NS(C)(=O)=O)cc2nc1N1C[C@@H](O)[C@H](NC(=O)OC(C)(C)C)C1]

To a solution of starting material (231.5 mg, 0.5 mmol, described in Example 4, Int-1) in anhydrous DMF (1 ml) was added DIPEA (129.2 mg, 1 mmol) and BOP reagent (243.3 mg, 0.55 mmol). The resulting mixture was heated at 70° C. for 10 minutes, then (3R,4S)-(4-hydroxy-pyrrolidin-3-yl)-carbamic acid tert-butyl ester (132 mg, 0.65 mmol) was added. The reaction was continued at 70° C. for 1 hour. It was directly purified by RPLC (50 g, 25 to 65% acetonitrile and water, using 0.1% TFA as modifier). Yield 264.4 mg, 81.7%. Ion found by LCMS: [M+H]⁺=648.2.

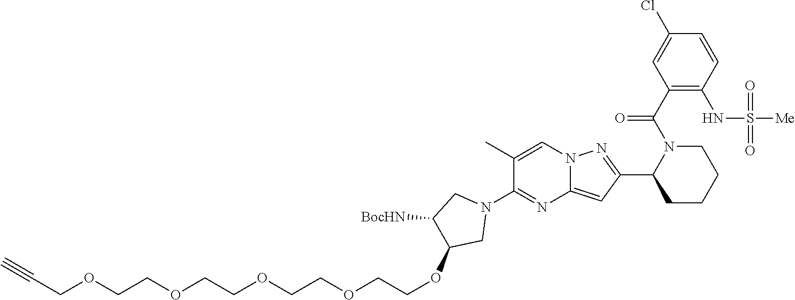 [SMILES: C#CCOCCOCCOCCOCCO[C@@H]1CN(c2nc3cc([C@@H]4CCCCN4C(=O)c4cc(Cl)ccc4NS(C)(=O)=O)nn3cc2C)C[C@H]1NC(=O)OC(C)(C)C]

Step b.

To a solution of the step-a product (104 mg, 0.162 mmol) in anhydrous DMF (1 ml) was added propargyl-PEG4-bromide (95.6 mg, 0.324 mmol) and potassium carbonate (44.8 mg, 0.324 mmol). The resulting mixture was heated at 100° C. for 5 hours. It was then purified by preparative HPLC (5% to 60% acetonitrile and water, using 0.1% TFA as modifier). Yield 48.9 mg, 35.1%. Ion found by LCMS: [M+H]⁺=862.2.

Step c.

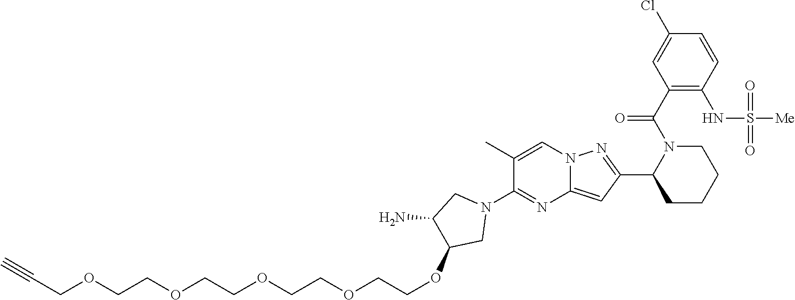 [SMILES: C#CCOCCOCCOCCOCCO[C@@H]1CN(c2nc3cc([C@@H]4CCCCN4C(=O)c4cc(Cl)ccc4NS(C)(=O)=O)nn3cc2C)C[C@H]1N]

The step-b product (48.9 mg, 0.0568 mmol) was dissolved in TFA (0.5 ml), and the solution was stirred at room temperature for 20 minutes. It was concentrated and purified by preparative HPLC (5% to 50% acetonitrile and water, using 0.1% TFA as modifier). Yield 28.5 mg, 57.3%. Ion found by LCMS: [M+H]⁺=762.2.

### Example 8. Synthesis of Int-5

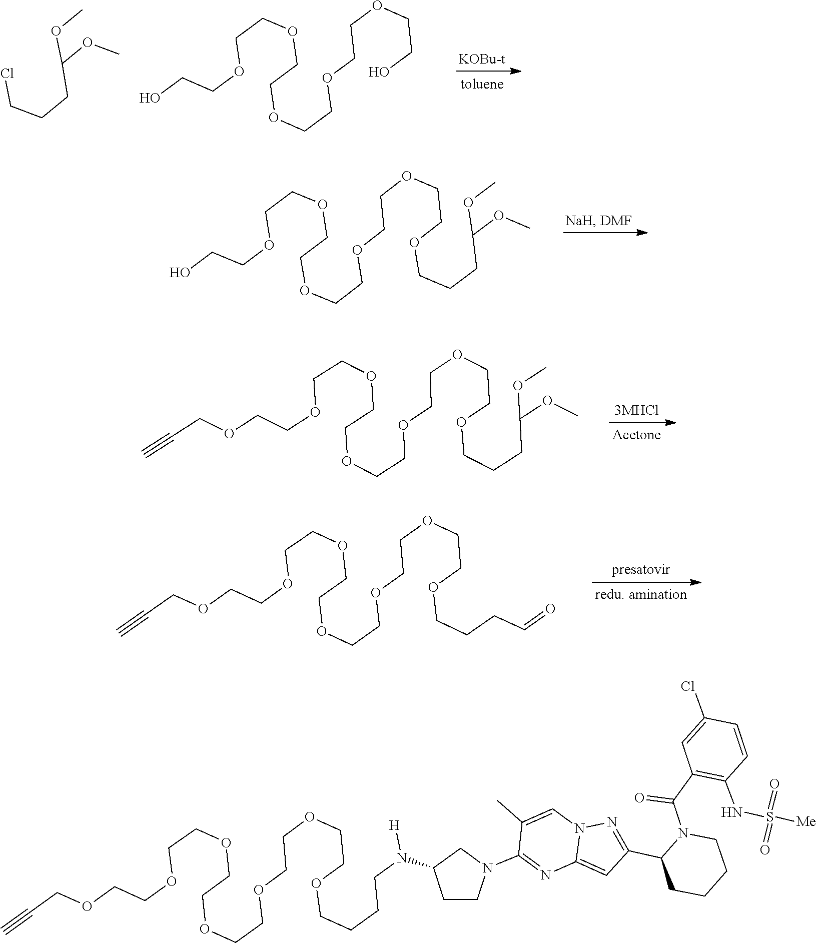

Step a.

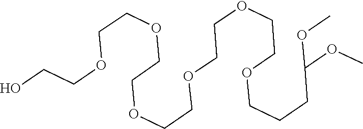 [SMILES: COC(CCCOCCOCCOCCOCCOCCOCCO)OC]

To a solution of hexaethylene glycol (5.6 g, 20 mmol) in anhydrous toluene (60 ml) was added potassium tert-butoxide (1.0 M, 22 ml, 22 mmol) in THF and 4-chlorobutyraldehyde diethyl acetal (3.6 g, 20 mmol). The mixture was stirred at 95-100° C. overnight under nitrogen atmosphere. After cooling to room temperature, the mixture was concentrated and purified by purified by column chromatography over silica eluted with 0% to 30% ethyl acetate and methanol. Yield of 6.1 g, 77%. Ion(s) found by LCMS: M+H=398.3.

Step b.

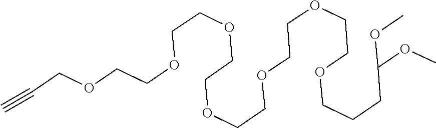 [SMILES: C#CCOCCOCCOCCOCCOCCOCCOCCCC(OC)OC]

To a solution of product from the previous step (0.8 g, 2.0 mmol) in anhydrous DMF was added sodium hydride (200 mg, 60% in oil, 5 mmol) and propargyl bromide (80 wt. % in toluene, 0.45 ml, 4 mmol) under the ice-water bath. The reaction was stirred for 4 hrs at room temperature under nitrogen atmosphere, then the mixture was concentrated and purified by column chromatography over silica eluted with 0% to 20% ethyl acetate and methanol. Yield of 0.7 g, 80%. Ion(s) found by LCMS: M+H=437.3.

Step c.

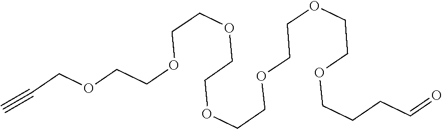 [SMILES: C#CCOCCOCCOCCOCCOCCOCCOCCCC=O]

To a solution of product from the previous step (0.7 g, 1.6 mmol) in 10 ml acetone was added HCl solution (3.0 M, 3.0 ml, 9.0 mmol). The progress of reaction was monitored by LCMS and the reaction was complete after 4 hours, and the reaction solution was concentrated to dryness and used in next step without further purification. Yield was quantitative for this step. Ion(s) found by LCMS: M/2+H=391.3.

Step d.

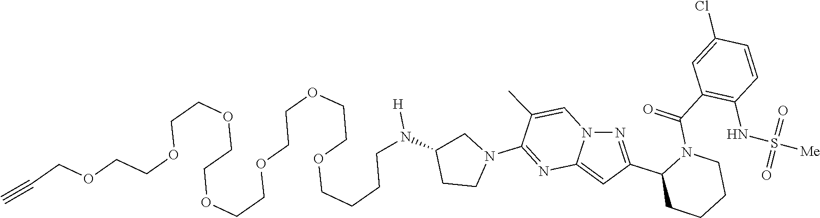 [SMILES: C#CCOCCOCCOCCOCCOCCOCCOCCCCN[C@H]1CCN(c2nc3cc([C@@H]4CCCCN4C(=O)c4cc(Cl)ccc4NS(C)(=O)=O)nn3cc2C)C1]

To a solution of products from previous step (0.4 g, 1 mmol) and presatovir (0.53 g, 1 mmol) in DCM (10 mL) was acetic acid (0.12 g, 2 mmol). The resulting solution was stirred for 1 hour at room temperature, then treated under vigorous stirring with sodium triacetoxyborohydride (0.42 g, 2 mmol). This mixture was stirred for overnight, then concentrated and purified by reverse phase liquid chromatography (RPLC) using an Isco COMBIFLASH® liquid chromatograph eluted with 5% to 80% acetonitrile and water with 0.1% TFA as modifier. Yield of the products 700 mg, 77.5%. Ion(s) found by LCMS: M/2+H=906.4.

### Example 9. Synthesis of Conjugate 4

Prepared the Click reagent solution: 0.0050 M CuSO₄ in PBS buffer solution: 10.0 mg CuSO₄ was dissolved in 12.53 mL PBS, then took 6.00 mL this CuSO₄ solution and added BTTAA (51.7 mg, CAS #1334179-85-9) and sodium ascorbate (297.2 mg) to give the Click reagent solution (0.0050M CuSO4, 0.020M BTTAA and 0.25M sodium ascorbate). This Click reagent solution was used for all of conjugations.

A solution of azido functionalized Fc (50.0 mg, 3.056 mL, 0.859 μmol, SEQ ID NO: 18 functionalized with PEG4-azide) was added to a 15 mL centrifuge tube containing alkyne derivatized small molecule (4.62 mg, 5.67 μmol, Int-6, 2 equivalents for each azido on the Fc). After gently shaking to dissolve all solids, the mixture was treated with the Click reagent solution (1.37 mL of L-ascorbic acid sodium, 0.25 M, 344 μmol, copper (II) sulfate 0.0050M, 6.87 μmol, and BTTAA 0.020M, 27.5 μmol). The resulting mixture was gently rotated for 6 hours at ambient temperature. It was purified by affinity chromatography over a protein A column, followed size exclusion chromatography (see general conjugate purification protocol). Maldi TOF analysis of the purified final product gave an average mass of 62021 Da (DAR=3.9). Yield 8.59 mg, 17% yield.

### Example 10. Syntheses of Conjugates

The preparation of the remaining conjugates was carried out analogously to Example 9, conjugate 4. The equivalents of TM were adjusted based on DAR values (2 equivalent per azido on Fc) and the Click reagent solution was used accordingly. The DAR values, molecular weight and yield are listed in Table 3.

TABLE 3
Conjugates and properties

| | | | | | MALDI | |
|---|---|---|---|---|---|---|
| | Azido | | Fc SEQ ID | DAR | mass Da | Yield |
| Conjugate | reagent | Int | NO: | (Average) | (Average) | (%) |
| 1 | PEG4-azide | 7 | 18 | 2.2 | 56971 | 43% |
| 2 | PEG4-azide | 7 | 35 | 7.8 | 55913 | 23% |
| 3a | PEG4-azide | 8 | 35 | 4.1 | 57278 | 51% |
| 3b | PEG4-azide | 8 | 35 | 7.4 | 62279 | 41% |
| 4 | PEG4-azide | 6 | 18 | 3.9 | 58908 | 17% |
| 5 | PEG4-azido | 8 | 35 | 5.2 | 60149 | 40% |
| | acetate | | | | | |
| 6 | PEG4-azide | 5 | 35 | 6.1 | 61726 | 25% |
| 7 | PEG4-azide | 25 | 35 | 5.5 | 61201 | 31% |
| 8 | PEG4-azido | 25 | 35 | 3.9 | 58289 | 44% |
| | acetate | | | | | |
| 9 | PEG4-azide | 27 | 35 | 7.6 | 69887 | 75% |
| 11 | PEG4-azide | 5 | 35 | 6.1 | 67096 | 13% |
| 12 | PEG4-azide | 10 | 35 | 3.2 | 56566 | 52% |
| 13 | PEG4-azide | 23 | 35 | 3.2 | 57089 | 58% |
| 14 | PEG4-azide | 40 | 35 | 4.8 | 59655 | 6% |
| 15a | PEG4-azide | 10 | 35 | 4.9 | 59074 | 5% |
| 15b | PEG4-azide | 10 | 35 | 5.3 | 59505 | 11% |
| 16 | PEG4-azide | 24 | 35 | 4.3 | 58524 | 4% |
| 17 | PEG4-azide | 26 | 35 | 4.5 | 58408 | 64% |
| 18 | PEG4-azide | 29 | 63 | 6.4 | 66101 | 55% |
| 19 | PEG4-azide | 42 | 64 | 3.0 | 61283 | 18% |

### Example 11. Activity of Pre-Conjugation Intermediates (Ints) and Conjugates in an RSV Cytopathic Effects (CPE) Assay

The activity of anti-RSV compounds and conjugates was determined using an in vitro cell-based assay following a standard protocol in the field. Briefly, ten four-fold serial dilutions of each test article (TA) starting at 10 μM were prepared in duplicate and incubated with RSV (Respiratory Syncytial Virus strain A2) at a multiplicity of infection (MOI) of 0.01, for one hour. The RSV-TA mix was then added to HEp-2 cells seeded in 96-well plates and incubated for one hour. On day four post incubation, cells were stained with crystal violet and optical density was read for calculation of 50 percent effective concentration (EC₅₀) of each TA using the XLfit dose response model.

In parallel with the CPE assay, the inherent cytotoxicity of several test articles was also determined. Briefly, ten four-fold serial dilutions of each TA starting at 10 μM were prepared in triplicate for inoculation with HEp-2 cells in 96-well culture plates. The cell viability was determined four days post treatment using CellTiter-Glo kit. 50% of cytotoxicity concentration (CC₅₀) was calculated using XLfit dose response model.

The results of the assays are summarized in Table 4. Test articles were compared to two benchmark compounds, Ribavirin and Presatovir. Importantly, all test articles had EC₅₀ values superior to the FDA approved drug Ribavirin. Two compounds, Int-7 and Int-8, were significantly more potent than the rest of the panel and approached the activity of Presatovir. Also of significance is that Conjugate 2 retains activity within 4-fold of the TM by itself. Lastly, the cytotoxicity (CC₅₀) of compounds Int-7 and Int-9 was greater than 180-fold, relative to their EC₅₀ values.

TABLE 4
RSV CPE Assay

| Test | EC50 | CC50 |
|---|---|---|
| article | (μM) | (μM or %) |
| DMSO | nt | 1.17% |
| Ribavirin | 25.85 | 173.6 |
| Presatovir | 0.0000769 | 25.89 |
| Int-6 | 2.199 | nt |
| Int-7 | 0.000353 | 10.4 |
| Int-8 | 0.00229 | nt |
| Int-9 | 0.2723 | 49.66 |
| Conjugate 1 | 0.3225 | nt |
| Conjugate 2 | 0.00121 | nt |
| Conjugate 3 | 0.1166 | nt |

nt = not tested. Values are an average of duplicate wells

### Example 12. RSV F Protein Binding Assay

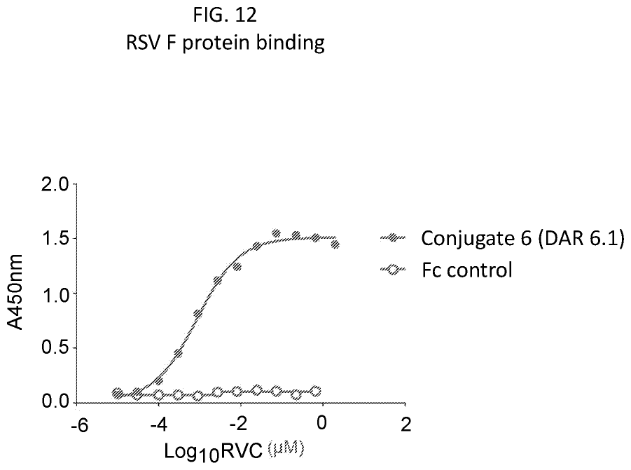

Nunc MaxiSorp flat-bottom 96-well plates (12-565-136, Fisher Scientific) were coated with recombinant RSV F protein (11049-V08B, Sino Biological) at 1 μg/mL in Seracare KPL coating solution (50-674-4, Fisher Scientific) at room temp for 1 h (100 μL, 0.1 μg/well) on an orbital microplate shaker at 500 rpm (BT908, BT LabSystems). Plates were washed (5×300 μL) with wash buffer (PBS 0.05% Tween 20) and blocked with 5% non-fat dry milk (9999S, Cell Signaling Technology) in wash buffer for 1 h at room temp with shaking. The blocking agent was removed and wells incubated with 3-fold serial dilutions of RVC in sample diluent (2.5% non-fat milk in PBS 0.025% Tween 20) starting at 2 μM for 2 h with shaking at room temp. After 5×300 μL washes, the plates were incubated with HRP conjugated donkey anti-human IgG Fc F(ab′)₂ (709-036-098, Jackson ImmunoResearch) secondary antibody diluted 1:1,000 in sample diluent for 1 h with shaking at room temp. Plates were then washed (8×300 μL) and developed with TMB substrate (BD555214, Fisher Scientific) for 3-5 minutes at room temp. The reaction was stopped with 1N H₂SO₄ and the absorbance read at 450 nm using the EnSpire multimode plate reader (PerkinElmer). Half maximal effective concentration (EC₅₀) was calculated with GraphPad Prism version 8 using nonlinear regression analysis (Sigmoidal, 4PL) of binding curves. Unconjugated Fc molecule was run as the negative control in each binding assay. The results are provided in FIG. 12 and Table 5.

TABLE 5
RSV F protein binding EC₅₀ (nM)

| | | Fc Carrier | | | Average |
|---|---|---|---|---|---|
| | | SEQ | | | EC₅₀ |
| Conjugate | Int | ID NO | Cross Linker | DAR | (nM) |
| n/a | n/a | 18 | | n/a | >2.0E+03 |
| n/a | n/a | 35 | | n/a | 6.2E+02 |
| Conjugate 1 | Int-7 | 18 | PEG4-PEG4 | 2.2 | 5.2E+02 |
| Conjugate 2 | Int-7 | 35 | PEG4-PEG4 | 7.8 | 1.2E+02 |
| Conjugate 3a | Int-8 | 35 | PEG8-PEG4 | 4.1 | 1.5E+02 |
| Conjugate 3b | Int-8 | 35 | PEG8-PEG4 | 7.4 | 4.9E+01 |
| Conjugate 4 | Int-6 | 18 | PEG3-PEG4 | 3.9 | 5.9E+00 |
| Conjugate 5 | Int-8 | 35 | PEG8-PEG0 | 5.2 | 6.1E+01 |
| Conjugate 6 | Int-5 | 35 | PEG7-PEG4 | 6.1 | 9.0E−01 |
| Conjugate 7 | Int-25 | 35 | PEG4-piperazine-C3-PEG4 | 5.5 | 5.2E+02 |
| Conjugate 8 | Int-25 | 35 | PEG4-piperazine-C3-PEG4 | 6.4 | 7.0E+02 |
| Conjugate 11 | Int-5 | 18 | PEG7-PEG4 | 7.0 | 1.6E+00 |
| Conjugate 12 | Int-10 | 35 | PEG4-piperazine-C3-PEG0 | 3.2 | 1.5E+00 |
| Conjugate 13 | Int-23 | 35 | PEG4-cysteric acid- | 3.2 | 1.2E+02 |
| | | | piperazine-C3-PEG1 | | |
| Conjugate 14 | Int-40 | 35 | PEG4-cysteric acid-hexanoic acid | 4.8 | 8.1E+01 |
| Conjugate 15a | Int-10 | 35 | PEG4-piperazine-C3-PEG4 | 4.9 | 3.0E+00 |
| Conjugate 15b | Int-10 | 35 | PEG4-piperazine-C3-PEG4 | 5.3 | 1.6E+00 |
| Conjugate 16 | Int-24 | 35 | PEG4-piperazine-C4-PEG4 | 4.3 | 2.2E+00 |
| Conjugate 17 | Int-26 | 35 | PEG8-laterial COOH-C4-PEG4 | 4.5 | 4.1E+02 |

### Example 13. General Procedure for Synthesis of Azido Fc

Preparation of PEG4-azido NHS ester solution (0.050 M) in DMF/PBS: 16.75 mg of PEG4-azido NHS ester was dissolved in 0.100 mL of DMF at 0° C. and diluted to 0.837 mL by adding PBS 1× buffer at 0° C. This solution was used for preparing other PEG4-azido Fc with a variety of DAR values by adjusting the equivalents of this PEG4-azido NHS ester PBS solution.

Pretreatment of h-IgG1 Fc, SEQ ID NO: 48 (107.2 mg in 8.800 mL of pH 7.4 PBS, MW-57891 Da, 1.852 μmol): The Fc solution was transferred into four centrifugal concentrators (30,000 MWCO, 15 mL) and diluted to 15 mL with PBS x1 buffer and concentrated to a volume of ˜1.5 mL. The residue was diluted 1:10 in PBS pH 7.4, and concentrated again. This wash procedure was repeated for total of four times followed by dilution to 8.80 mL.

Preparation of PEG4-azido Fc: 0.050M PEG4-azidoNHS ester PBS buffer solution (0.593 mL, 29.6 μmol, 16 equivalents) was added to above solution of h-IgG1 Fc (SEQ ID NO: 48) and the mixture was shaken rotated for 2 hours at ambient temperature. The solution was concentrated by using four centrifugal concentrators (30,000 MWCO, 15 mL) to a volume of ˜1.5 mL. The crude mixture was diluted 1:10 in PBS pH 7.4, and concentrated again. This wash procedure was repeated for total of three times. The concentrated Fc-PEG4-azide was diluted to 8.80 mL with pH 7.4 PBS buffer and ready for Click conjugation. The purified material was quantified using a NANODROP™ UV visible spectrophotometer (using a calculated extinction coefficient based on the amino acid sequence of h-IgG1). Yield was quantitative after purification.

### Example 14. Synthesis of Conjugate 6

A solution of azido functionalized Fc (75 mg, 7.5 mL, 1.406 umol, Example 13; SEQ ID NO: 35 functionalized with PEG4-azide) was added to a 40 mL centrifuge tube containing alkyne functionalized small molecule (12.2 mg, 13.49 umol, described in Example 8, Int-5). After gently agitating to dissolve all solids, the solution was added to a solution of L-ascorbic acid sodium salt (22.2 mg, 112.4 umol), copper (II) sulfate (4.3 mg, 27.0 umol), and THPTA (46.9 mg, 108.0 umol) in PBS 7.4 buffer (15.5 mL, 1×). The resulting solution was gently rotated overnight. It was purified by affinity chromatography over a protein A column, followed size exclusion chromatography (See conjugate purification protocol). Maldi TOF analysis of the purified final product gave an average mass of 60,658 Da (DAR=6.1). Yield 15.0 mg, 25% yield.

### Example 15. Synthesis of Int-8

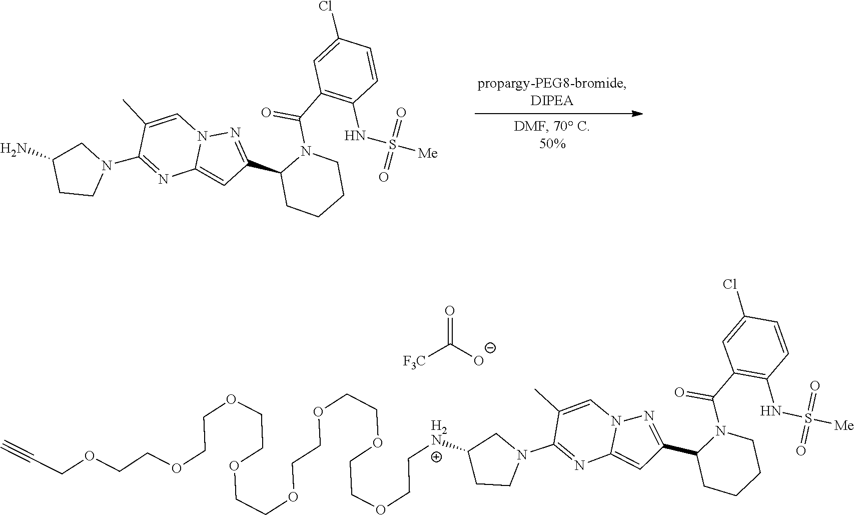

A stirring solution of presatovir (100 mg, 0.212 mmol), propargyl-PEG8-bromide (169 mg, 0.318 mmol), and N,N-diisopropylethylamine (74 uL, 0.424 mmol), was heated to 70° C. for 12 h. Additional propargyl-PEG8-bromide (169 mg, 0.318 mmol) and N,N-diisopropylethylamine (148 uL, 0.848 mmol) were added and heated at 50° C. was continued for 24 h. All the volatiles were removed per vacuum techniques. The residue was purified by HPLC (0 to 90% methanol and water). Yield 109 mg, 50%. Ions found by LCMS: [(M+H)]+=922.2.

### Example 16. Synthesis of Conjugate 3a

A solution of azido functionalized Fc (100 mg, 10 mL, 1.790 umol, Example 13; SEQ ID NO: 35 functionalized with PEG4-azide) was added to a 40 mL centrifuge tube containing alkyne functionalized small molecule (11.7 mg, 11.24 umol, described in Example 15, Int-8). After gently agitating to dissolve all solids, the solution was treated with a solution of L-ascorbic acid sodium salt (37.1 mg, 187.4 umol), copper (II) sulfate (4.0 mg, 25.0 umol), and BTTAA (38.73 mg, 89.95 umol) in PBS 7.4 buffer (15.0 mL). The resulting mixture was gently rotated overnight. It was purified by affinity chromatography over a protein A column, followed size exclusion chromatography (See conjugate purification protocol). Maldi TOF analysis of the purified final product gave an average mass of 58,395 Da (DAR=4.1). Yield 25.5 mg, 51% yield.

### Example 17. Synthesis of Conjugate 3b

A solution of azido functionalized Fc (75 mg, 7.5 mL, 1.406 umol, Example 13; SEQ ID NO: 35 functionalized with PEG4-azide) was added to a 40 mL centrifuge tube containing alkyne functionalized small molecule (17.5 mg, 16.87 umol, described in Example 15, Int-8). After gently agitating to dissolve all solids, the solution was added to a solution of L-ascorbic acid sodium salt (27.8 mg, 140.6 umol), copper (II) sulfate (5.4 mg, 33.7 umol), and THPTA (58.6 mg, 134.9 umol) in PBS 7.4 buffer (15.5 mL, 1×). The resulting solution was gently rotated overnight. It was purified by affinity chromatography over a protein A column, followed size exclusion chromatography (See conjugate purification protocol). Maldi TOF analysis of the purified final product gave an average mass of 62,279 Da (DAR=7.4). Yield 30.6 mg, 41% yield.

### Example 18. Synthesis of Conjugate 5

A solution of azido functionalized Fc (75 mg, 7.5 mL, 1.406 umol, Example 2; SEQ ID NO: 35 functionalized with PEG4-azido acetate) was added to a 40 mL centrifuge tube containing alkyne functionalized small molecule (17.5 mg, 16.87 umol, described in Example 15, Int-8). After gently agitating to dissolve all solids, the solution was added to a solution of L-ascorbic acid sodium salt (27.8 mg, 140.6 umol), copper (II) sulfate (5.4 mg, 33.7 umol), and THPTA (58.6 mg, 134.9 umol) in PBS 7.4 buffer (15.5 mL, 1×). The resulting solution was gently rotated overnight. It was purified by affinity chromatography over a protein A column, followed size exclusion chromatography (See conjugate purification protocol). Maldi TOF analysis of the purified final product gave an average mass of 58,660 Da (DAR=5.2). Yield 29.7 mg, 40% yield.

### Example 19. Synthesis of Int-10

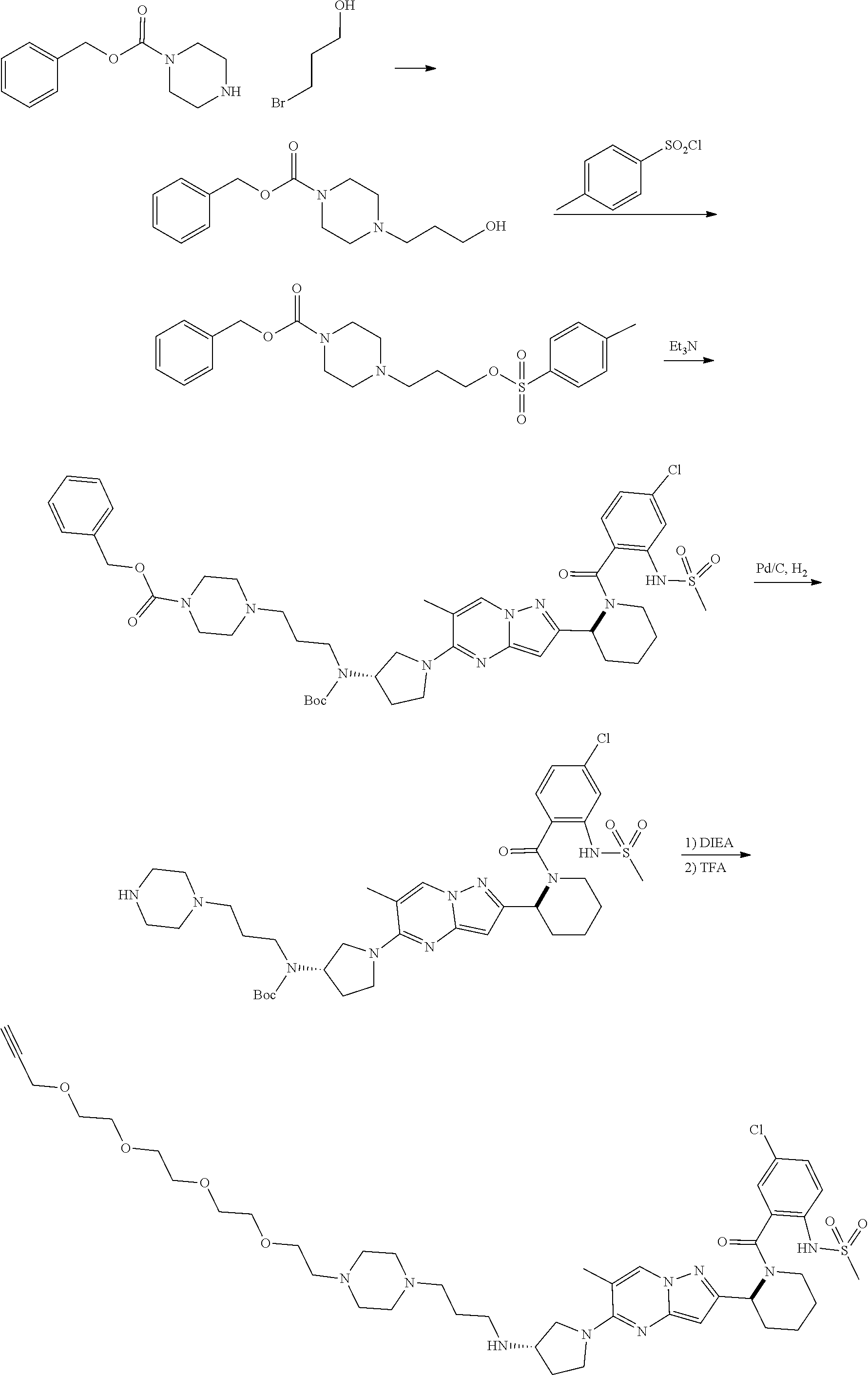

Step a.

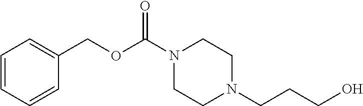 [SMILES: O=C(OCc1ccccc1)N1CCN(CCCO)CC1]

Cbz-Piperazine (3.5 g, 15.9 mmol), 3-bromo-1-propanol (2.7 g, 19.1 mmol), and triethylamine (1.9 g, 19.1 mmol) were stirred in DMF (15 mL) at room temperature for 12 hours. Half of the solvent was removed by rotary evaporator. DI water was added and the mixture was extracted with ethyl acetate (3×30 mL). The combined organic extracts were washed with brine and dried over sodium sulfate. The solvent was removed and the residue was purified by silica gel chromatography (0% to 80% ethyl acetate and hexanes) to afford the product as a thick clear oil. Yield 87%. LC/MS [M+H]+=279.2.

Step b.

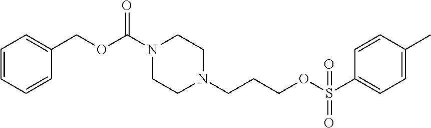 [SMILES: Cc1ccc(S(=O)(=O)OCCCN2CCN(C(=O)OCc3ccccc3)CC2)cc1]

The intermediate from step a (4 g, 14.4 mmol), tosyl chloride (3.6 g, 18.7 mmol), and triethylamine (2.6 g, 25.9 mmol) were stirred in DCM (50 mL) at room temperature for 12 hours. DI water was added and the mixture was extracted with DCM (3×30 mL). The combined organic extracts were washed with brine and dried over sodium sulfate. The solvent was removed and the residue was purified by silica gel chromatography (0% to 10% methanol in DCM) to afford the product as a white solid. Yield 67%. LC/MS [M+H]+=433.2.

Step c.

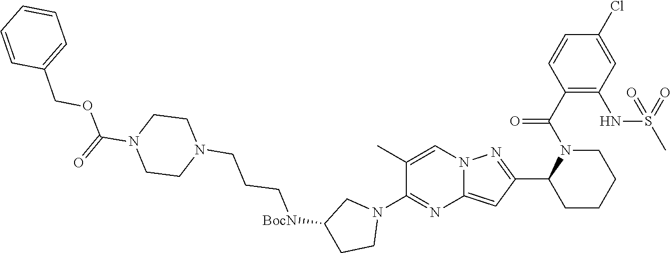 [SMILES: Cc1cn2nc([C@@H]3CCCCN3C(=O)c3ccc(Cl)cc3NS(C)(=O)=O)cc2nc1N1CC[C@H](N(CCCN2CCN(C(=O)OCc3ccccc3)CC2)C(=O)OC(C)(C)C)C1]

The intermediate from step b (0.35 g, 0.81 mmol), Presatovir TFA salt (0.26 g, 0.41 mmol), and triethylamine (0.16 g, 1.6 mmol) were stirred in DMF (3 mL) at room temperature for 80° C. for 45 minutes. The product was observed by LC/MS [M+H]+=792.2. The mixture was cooled to room temperature and Boc anhydride (57 mg, 0.81 mmol) was added. The mixture stirred at room temperature for 1 hour, then solvent was removed, and the residue was purified by reversed phase Isco COMBIFLASH® chromatography (20% to 95% acetonitrile in DI water, 0.1% TFA, 30 minute gradient). The pure fractions were pooled and lyophilized to afford the product as a light pink solid. Yield 81%. LC/MS [M+H]+=892.2.

Step d.

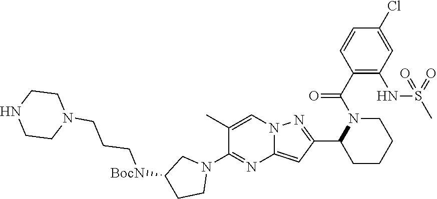

The intermediate from step c (0.21 g, 0.21 mmol) was stirred in methanol under 1 atm of hydrogen in the presence of 5% Pd/C (50 mg) for 30 minutes. The mixture was filtered through celite, concentrated and dried under high vacuum. Yield=99%. LC/MS [M+H]+=758.2.

Step e.

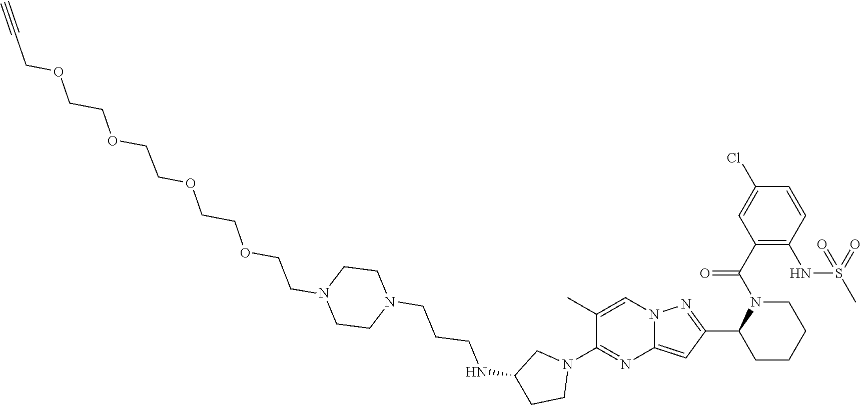 [SMILES: C#CCOCCOCCOCCOCCN1CCN(CCCN[C@H]2CCN(c3nc4cc([C@@H]5CCCCN5C(=O)c5cc(Cl)ccc5NS(C)(=O)=O)nn4cc3C)C2)CC1]

The intermediate from step d of this example was dissolved in DMF (2 mL), mixed with propargyl-PEG4-bromide (91 mg, 0.28 mmol), and N,N-diisopropylethylamine (71 mg, 0.55 mmol then stirred at 80° C. for 3 hours. The mixture was cooled and purified directly by reversed phase Isco COMBIFLASH® chromatography (20% to 95% acetonitrile in DI water, 0.1% TFA, 30 minute gradient). The pure fractions were pooled and lyophilized to afford the product as a light pink solid. LC/MS [M+H]+=972.2.

This boc-protected intermediate was stirred in TFA (3 mL) at room temperature for 30 minutes. The solvent was removed by rotovap and purified by reversed phase HPLC chromatography ISCO ACQ semi prep (20-95% acetonitrile in DI water, 0.1% TFA, 30 minute gradient). The pure fractions were pooled and lyophilized to afford the product as a light pink solid. Yield 41%. LC/MS [M+H]+=872.2.

### Example 20. Synthesis of Int-23

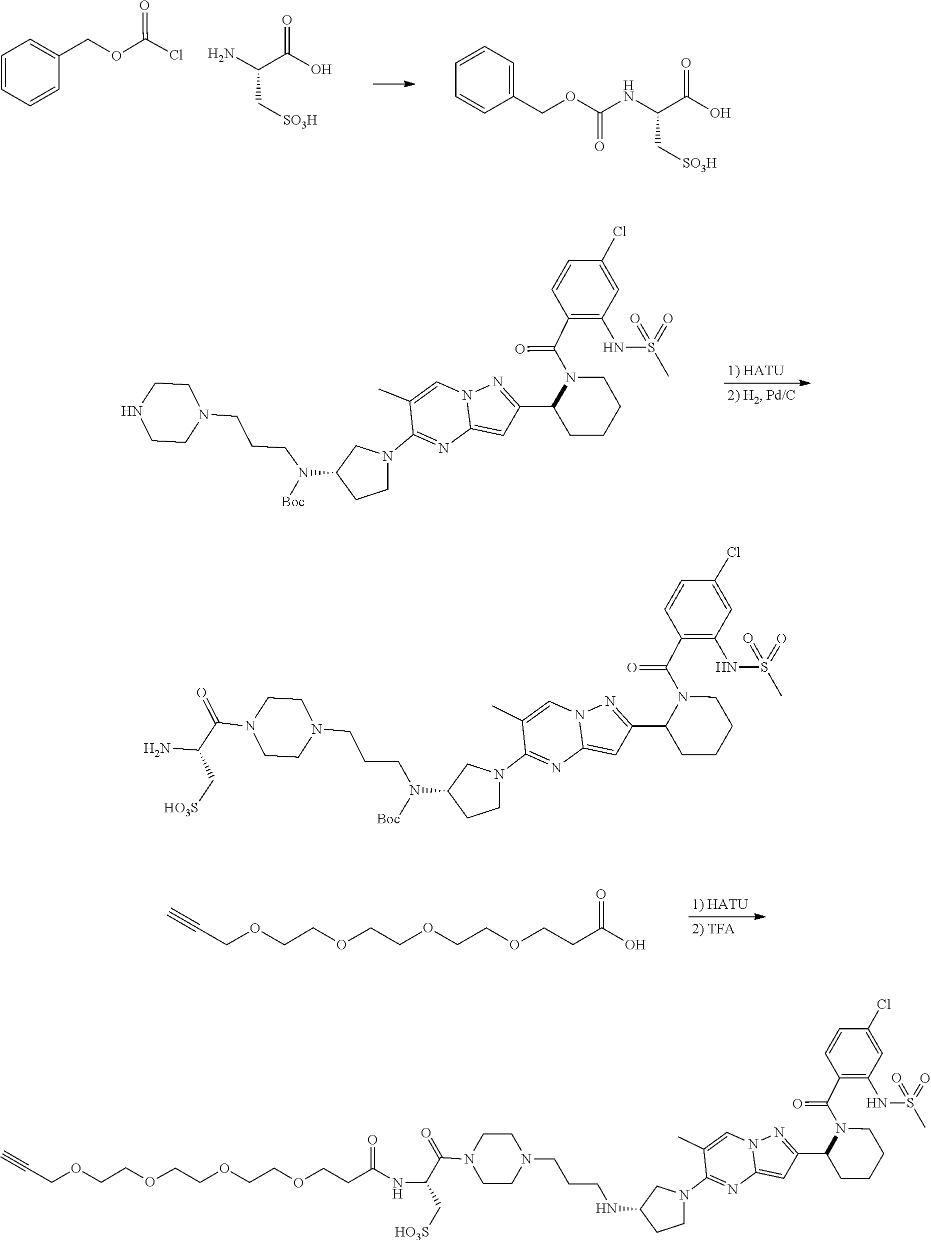

Step a.

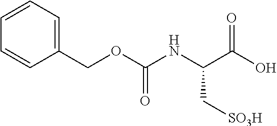 [SMILES: O=C(N[C@@H](CS(=O)(=O)O)C(=O)O)OCc1ccccc1]

Benzyl chloroformate was added dropwise to a stirred mixture of cysteic acid and triethylamine in ACN/saturated aqueous sodium bicarbonate (1/1) cooled to 0° C. The reaction was stirred at 0° C. for 40 minutes and most of the solvent was removed on the rotary evaporator. The crude material was purified by reversed phase Isco COMBIFLASH® HPLC chromatography (5% to 80% ACN in DI water, 0.1% TFA, 30 minute gradient). The pure fractions were cooled and concentrated to afford the crude product as a clear, viscous oil. Yield 58%. Ion found by LC/MS [M−H]⁻=302.2

Step b.

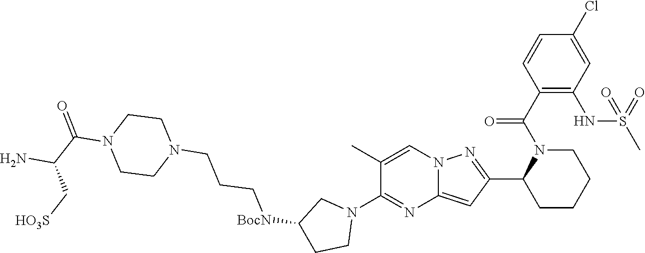 [SMILES: Cc1cn2nc([C@@H]3CCCCN3C(=O)c3ccc(Cl)cc3NS(C)(=O)=O)cc2nc1N1CC[C@H](N(CCCN2CCN(C(=O)[C@@H](N)CS(=O)(=O)O)CC2)C(=O)OC(C)(C)C)C1]

HATU (156 mg, 0.41 mmol) was added to a stirring mixture of the intermediate described in step a of this example (125 mg, 0.41 mmol), the intermediate described in step d of Example 19, Int-10 (240 mg, 0.32 mmol), and triethylamine (127 mg, 1.27 mmol) in DMF (4 mL). The reaction was stirred for 4 hours at room temperature. The solvent was reduced by half on the rotary evaporator and purified by reversed phase chromatography using an Isco COMBIFLASH® (5-80% ACN in DI water, 0.1% TFA, 30 minute gradient). The pure fractions were pooled and concentrated to afford the product as an oil, LC/MS ([M/2)=H]+=522.2.

This intermediate was stirred in methanol (15 ml) under 1 atm of hydrogen gas, in the presence of 5% Pd/C (30 mg) for 2 hours. The mixture was filtered through celite and concentrated to afford the product as a viscous oil. Yield 43%, 2 steps. Ion found by LC/MS [M+H]+=909.2.

Step c.

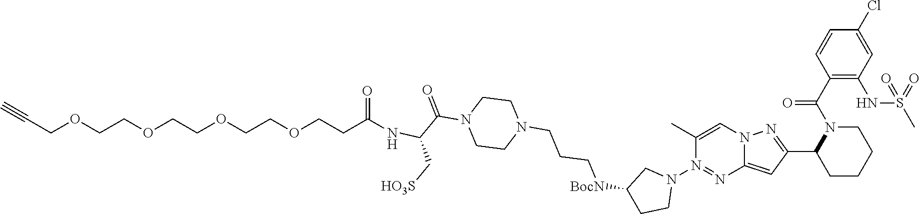

HATU (92 mg, 0.24 mmol) was added to a stirring mixture of the intermediate described in step b. of this example (170 mg, 0.19 mmol), propargyl-peg4-carboxylic acid (63 mg, 0.24 mmol), and triethylamine (94 mg, 0.93 mmol) in DMF (3 mL). The reaction was stirred at ambient temperature for 4 hours. The solvent was reduced by half on a rotary evaporator and the residue was purified by reversed phase chromatography Isco COMBIFLASH® HPLC (10% to 90% acetonitrile in DI water, 0.1% TFA, 30 minute gradient). Ion found by LC/MS [(M/2)+H]+=576.2.

The boc protected intermediate was stirred in a 1/1 mixture of DCM/TFA (5 mL) for 45 minutes. The solvent was removed by rotary evaporator and purified by reversed phase HPLC on an ISCO ACCQ semi prep (5% to 75% CAN in DI water, 0.1% TFA, 30 minute gradient). The pure fractions were pooled and lyophilized to afford the product as an off white solid. Yield 48%, 2 steps. Ion found by LC/MS [(M/2)+H]+=526.2.

### Example 21. Synthesis of Int-24

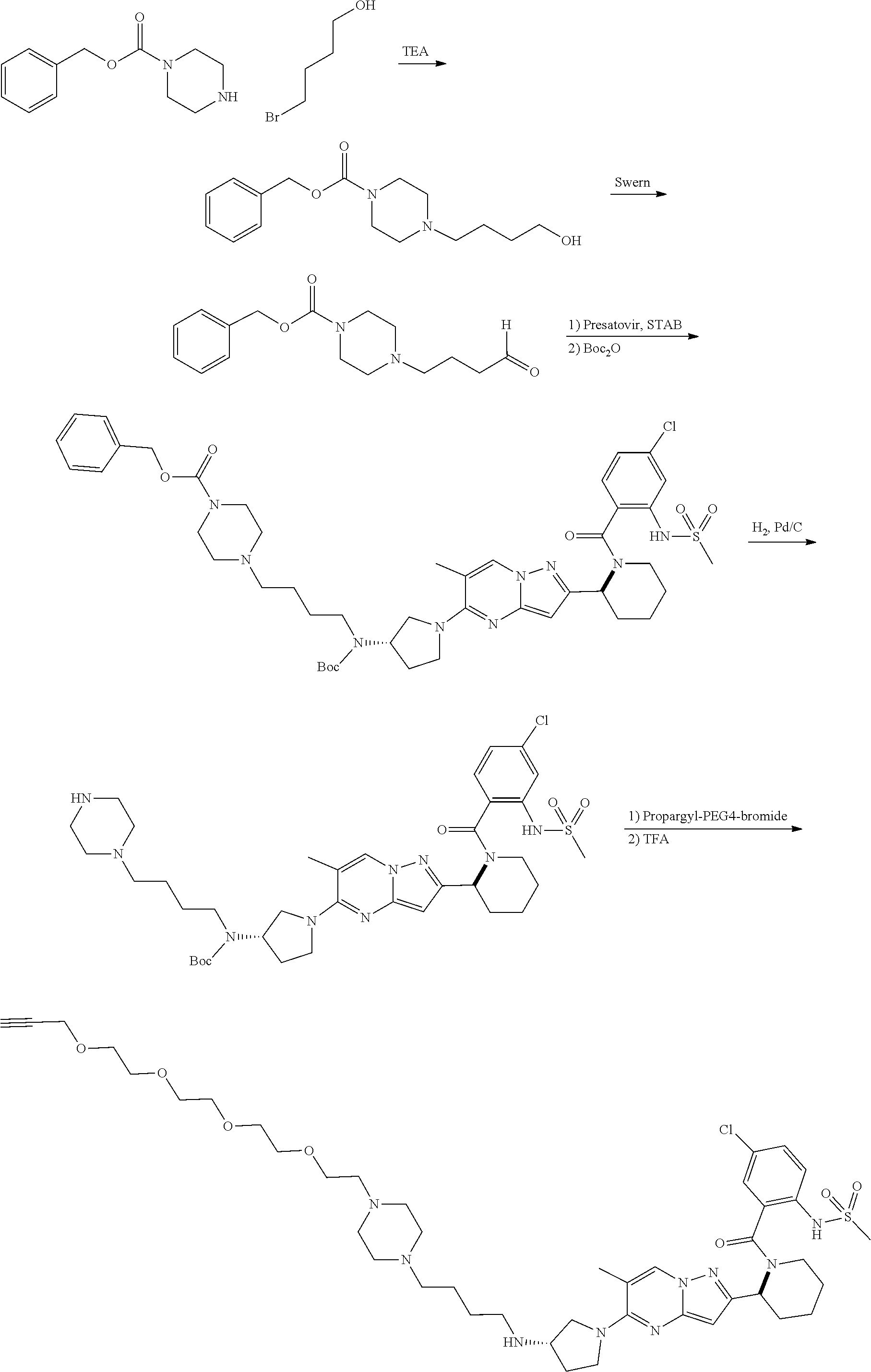

Step a.

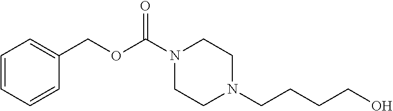 [SMILES: O=C(OCc1ccccc1)N1CCN(CCCCO)CC1]

CBZ-Piperazine (4 g, 18.2 mmol), n-bromo-butanol (4.2 g, 27.2 mmol), and triethylamine (3.7 g, 36.3 mmol) were stirred in DMF (15 mL) at 60° C. for 12 hours. Half of the solvent was removed by rotary evaporator. DI water was added and the mixture was extracted with ethyl acetate (3×, 30 mL). The combined organic extracts were washed with brine and dried over sodium sulfate. The solvent was removed and the residue was purified by silica gel chromatography (0% to 5% methanol in DCM) to afford the product as a thick clear oil. Yield 77%. LC/MS [M+H]+=293.2.

Step b.

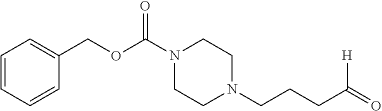 [SMILES: O=CCCCN1CCN(C(=O)OCc2ccccc2)CC1]

Oxalyl chloride was added to DCM (15 mL) cooled to −78° C. (dry ice/acetone bath) under an atmosphere of nitrogen. DMSO (801 mg, 10.26 mmol in 5 m of DCM was added to the oxalyl chloride solution dropwise via syringe over a period of 5 minutes. The mixture was stirred at −78° C. for 10 minutes at which point the intermediate alcohol described in step a of this example (1.5 g, 5.13 mmol, in 5 mL DCM) was added dropwise via syringe over a period of 5 minutes. The mixture was stirred at −78° C. for 30 minutes. Triethylamine (2.6 g, 25.7 mmol, in 10 mL of DCM) was added dropwise via syringe over a 10 minute period. The reaction was stirred for an additional 10 minutes at −78° C. and then gradually warmed to ambient temperature over a period of 1 hour. The mixture was diluted with DI water and extracted into DCM (3×30 mL). The combined organic extracts were washed with brine, dried over sodium sulfate, filtered and concentrated to afford the product as a viscous oil which was taken forward without further purification. Yield 91%. LC/MS [M+H]+=291.2.

Step c.

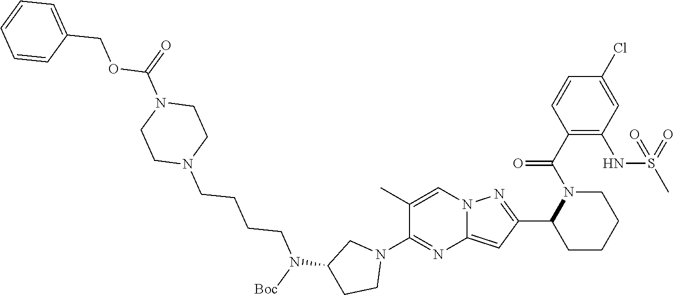

The intermediate from step b of this example (327 mg, 0.75 mmol), Presatovir TFA salt (400 mg, 0.75 mmol), and triethylamine (151 mg, 1.5 mmol), and sodium triacetoxy borohydride (397 mg, 1.88 mmol) were stirred in methanol (25 mL) at room temperature for 1 hour. Glacial acetic acid was added (˜1 mL) and the solvent was removed by rotary evaporator. The residue was purified by Isco COMBIFLASH® reversed phase chromatography (5% to 95% ACN in DI water, 0.1% TFA). The solvent was removed by rotary evaporator. Ions found by LC/MS [M+H]+=806.4.

This intermediate was dissolved in methanol (20 mL), triethylamine (81 mg, 0.81 mmol), and boc anhydride (328 mg, 0.81 mmol), then stirred at room temperature for 1 hour. The solvent was removed, and then the resulting residue was purified by reversed phase chromatography Isco COMBIFLASH® (20% to 95% acetonitrile in DI water, 0.1% TFA, 30 minute gradient). The pure fractions were pooled and lyophilized to afford the desired product as a white solid. Yield 490 mg, 72%, 2 steps. Ion found by LC/MS [M+H]+=906.4.

Step d.

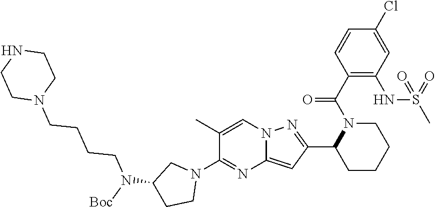

The intermediate from step c of this example (490 mg, 0.54 mmol) was stirred in methanol under 1 atm of hydrogen in the presence of 5% Pd/C (90 mg) for 30 minutes. The mixture was filtered through celite, concentrated and dried under high vacuum. Crude yield 391 mg, 94%. Ion found by LC/MS [M+H]+=772.2.

Step e.

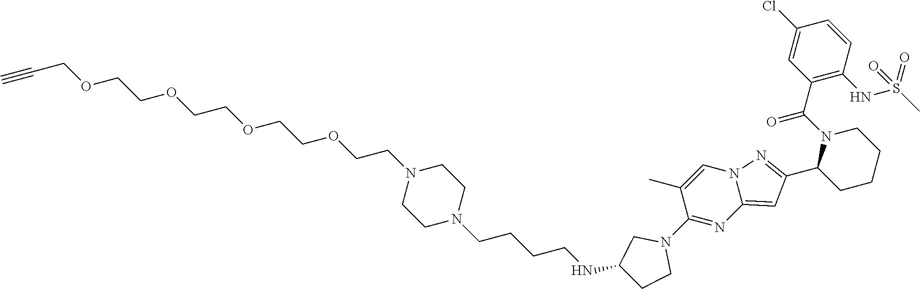 [SMILES: C#CCOCCOCCOCCOCCN1CCN(CCCCN[C@H]2CCN(c3nc4cc([C@@H]5CCCCN5C(=O)c5cc(Cl)ccc5NS(C)(=O)=O)nn4cc3C)C2)CC1]

The intermediate from step d (282 mg, 0.37 mmol) was dissolved in DMF (2 mL), then treated with propargyl-peg4-bromide (140 mg, 0.47 mmol), and N,N-diisopropylethylamine (188 mg, 1.5 mmol) and stirred at 80° C. for 3 hours. The mixture was cooled and purified directly by reversed phase Isco COMBIFLASH® chromatography (10% to 95% acetonitrile in DI water, 0.1% TFA, 30 minute gradient). The pure fractions were pooled and concentrated. Ion found by LC/MS [(M/2)+H]+=493.8.

The boc-protected intermediate was stirred in TFA (3 mL) at room temperature for 30 minutes. The solvent was removed by the rotary evaporator and purified by reversed phase HPLC chromatography ISCO ACCQ semi prep (20-95% acetonitrile in DI water, 0.1% TFA, 30 minute gradient). The pure fractions were pooled and lyophilized to afford the desired product as an off white solid. Yield 137 mg, 42%, 2 steps. Ion found by LC/MS [M+H]+=886.4.

### Example 22. Synthesis of Int-25

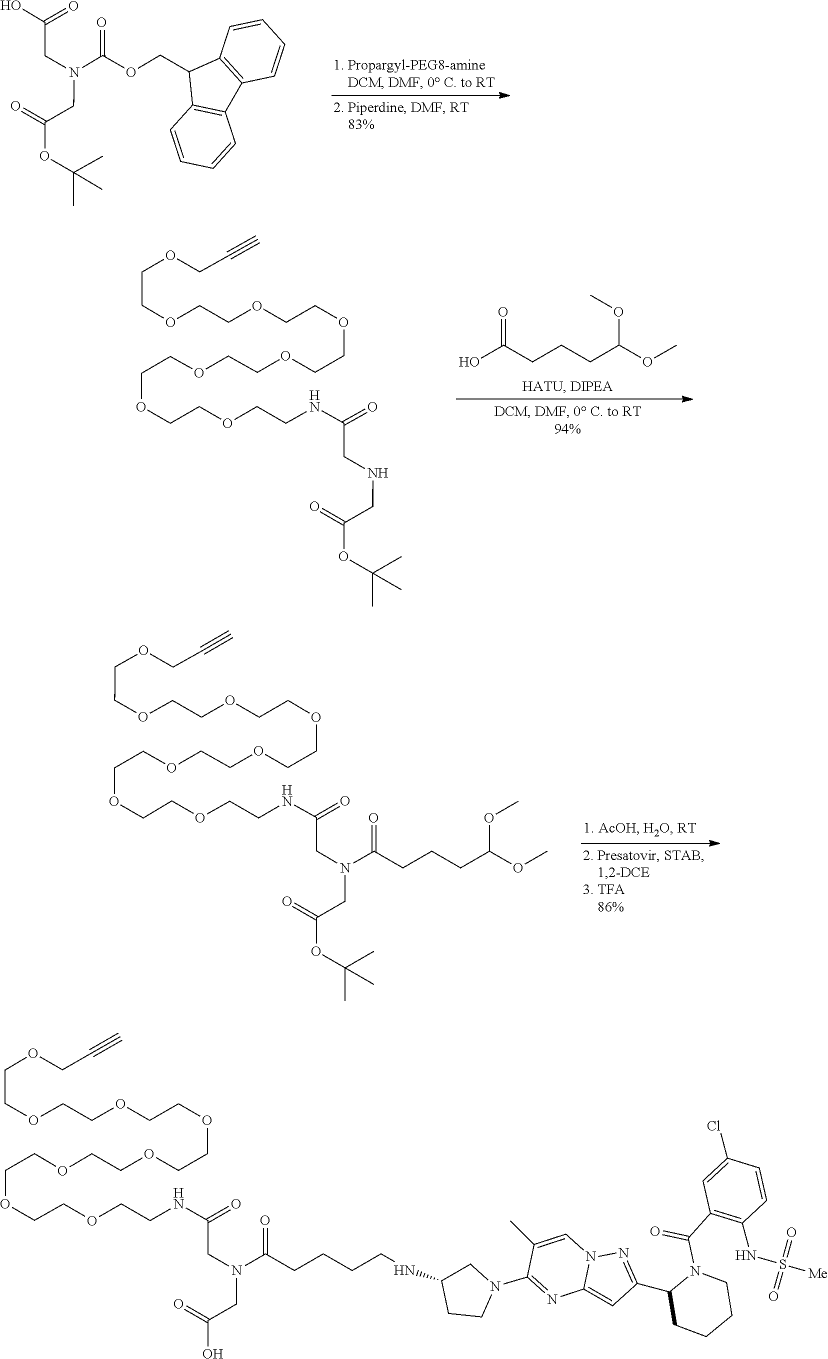 [SMILES: CC(C)(C)OC(=O)CN(CC(=O)O)C(=O)OCC1c2ccccc2-c2ccccc21 ; C#CCOCCOCCOCCOCCOCCOCCOCCOCCNC(=O)CNCC(=O)OC(C)(C)C ; COC(CCCC(=O)O)OC ; C#CCOCCOCCOCCOCCOCCOCCOCCOCCNC(=O)CN(CC(=O)OC(C)(C)C)C(=O)CCCC(OC)OC ; C#CCOCCOCCOCCOCCOCCOCCOCCOCCNC(=O)CN(CC(=O)O)C(=O)CCCCN[C@H]1CCN(c2nc3cc([C@@H]4CCCCN4C(=O)c4cc(Cl)ccc4NS(C)(=O)=O)nn3cc2C)C1]

Step a.

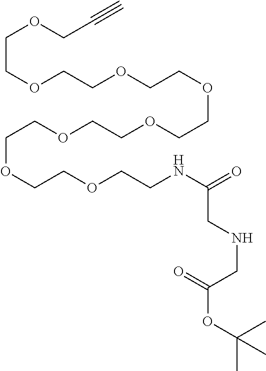 [SMILES: C#CCOCCOCCOCCOCCOCCOCCOCCOCCNC(=O)CNCC(=O)OC(C)(C)C]

To a 0° C. stirring solution of Fmoc-N-(tert-butyloxycarbonylmethyl)-glycine (336 mg, 0.818 mmol), propargyl-PEG8-amine (333 mg, 0.818 mmol) and DIPEA (356 uL, 2.045 mmol) in DMF (2.0 mL) and DCM (2.0 mL), was added HATU (317 mg, 0.834 mmol). The temperature was raised to ambient and stirring was continued until the reaction was complete as determined by LCMS. All the volatiles were removed per rotatory evaporation. The residue was purified by RP-C18 column using an Isco COMBIFLASH® liquid chromatography eluted with 0% to 100% water and methanol. The fractions containing the desired product were evaporated per rotatory evaporation, affording 720 mg of a material containing the desired product, but not in high purity (main observed ion found by LCMS: [(M+H)]⁺=801.2). This material was taken up in 5% piperidine in DMF (10 mL). All the volatiles were removed per rotatory evaporation. The residue was purified by RP-C18 column using an Isco COMBIFLASH® liquid chromatography eluted with 0% to 100% water and methanol. Yield 0.392 g, 83%. Ions found by LCMS: [(M+H)]⁺=579.2.

Step b.

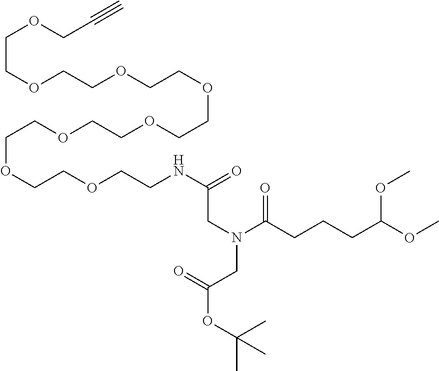 [SMILES: C#CCOCCOCCOCCOCCOCCOCCOCCOCCNC(=O)CN(CC(=O)OC(C)(C)C)C(=O)CCCC(OC)OC]

To a 0° C. stirring solution of the product from step a (250 mg, 0.432 mmol), 5,5-dimethoxyvaleric acid (77 mg, 0.475 mmol) and DIPEA (226 uL, 1.296 mmol) in DMF (3.0 mL) and DCM (0.5 mL), was added HATU (168 mg, 0.440 mmol). The temperature was raised to ambient and stirring was continued overnight. All the volatiles were removed per rotatory evaporation. The residue was purified by RP-C18 column using an Isco COMBIFLASH® liquid chromatography eluted with 0% to 100% water and methanol. Yield 0.294 g, 94%. Ions found by LCMS: [(M+H)−MeOH]⁺=692.1.

Step c.

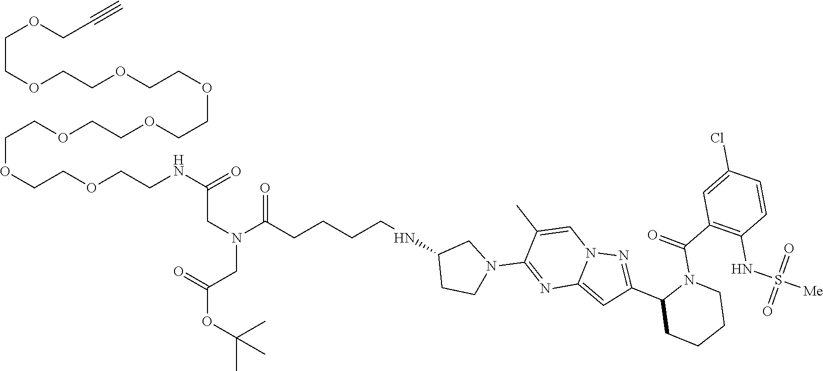 [SMILES: C#CCOCCOCCOCCOCCOCCOCCOCCOCCNC(=O)CN(CC(=O)OC(C)(C)C)C(=O)CCCCN[C@H]1CCN(c2nc3cc([C@@H]4CCCCN4C(=O)c4cc(Cl)ccc4NS(C)(=O)=O)nn3cc2C)C1]

The product from step b (294 mg, 0.407 mmol) dissolved in acetic acid (3.0 mL) and water (1.5 mL), and stirred until full conversion to the desired aldehyde was observed by analytical HPLC. All the volatiles were evaporated. The aldehyde was used in the next step without further purification. Yield 0.275 g, quant. Ions found by LCMS: [(M+H)]⁺=677.2.

To a stirring solution of the aldehyde (64 mg, 0.094 mmol), and presatovir (50 mg, 0.094 mmol) in 1,2-dichloroethane (3.0 mL), it was added sodium triacetoxyborohydride (30 mg, 0.141 mmol). LC-MS analysis after 12 hr showed the correct mass the desired product ([(M+2H)/2]+=596.8). All volatiles were removed per rotatory evaporation. The obtained residue was taken up in TFA (2.0 mL), and stirring was continued until full deprotection of the t-butyl ester was observed by HPLC. All the volatiles were removed per rotatory evaporation. The residue was purified by RP-C18 column using an Isco COMBIFLASH® liquid chromatography eluted with 0% to 100% water and methanol. Yield 115 mg, 86%. Ions found by LCMS: [(M+2H)/2]+=568.8

### Example 23. Synthesis of Conjugate 7

A solution of azido functionalized Fc (100 mg, 10 mL, 1.874 umol, Example 13; SEQ ID NO: 35 functionalized with PEG4-azide) was added to a 40 mL centrifuge tube containing alkyne functionalized small molecule (28.1 mg, 22.49 umol, described in Example 22, Int-25). After gently agitating to dissolve all solids, the solution was added to a solution of L-ascorbic acid sodium salt (37.1 mg, 187.4 umol), copper (II) sulfate (7.2 mg, 45.0 umol), and THPTA (78.2 mg, 179.9 umol) in PBS 7.4 buffer (20.62 mL, 1×). The resulting solution was gently rotated overnight. It was purified by affinity chromatography over a protein A column, followed size exclusion chromatography (See conjugate purification protocol). Maldi TOF analysis of the purified final product gave an average mass of 61,201 Da (DAR=5.5). Yield 39.2 mg, 31% yield.

### Example 24. Synthesis of Conjugate 8

A solution of azido functionalized Fc (100 mg, 10 mL, 1.874 umol, Example 2; SEQ ID NO: 35 functionalized with PEG4-azido acetate) was added to a 40 mL centrifuge tube containing alkyne functionalized small molecule (28.1 mg, 22.49 umol, described in Example 22, Int-25). After gently agitating to dissolve all solids, the solution was added to a solution of L-ascorbic acid sodium salt (37.1 mg, 187.4 umol), copper (II) sulfate (7.2 mg, 45.0 umol), and THPTA (78.2 mg, 179.9 umol) in PBS 7.4 buffer (20.62 mL, 1×). The resulting solution was gently rotated overnight. It was purified by affinity chromatography over a protein A column, followed size exclusion chromatography (See conjugate purification protocol). Maldi TOF analysis of the purified final product gave an average mass of 58,289 Da (DAR=3.9). Yield 44.1 mg, 44% yield.

### Example 25. Synthesis of Int-26

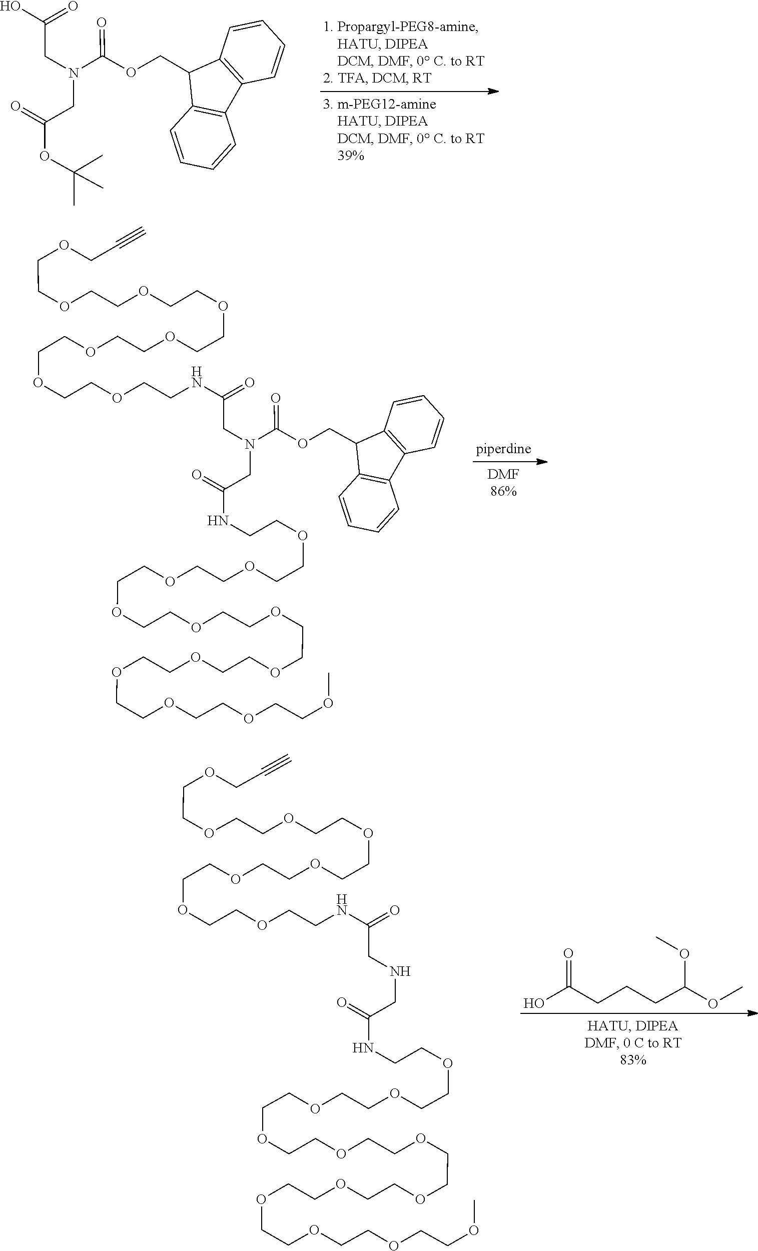

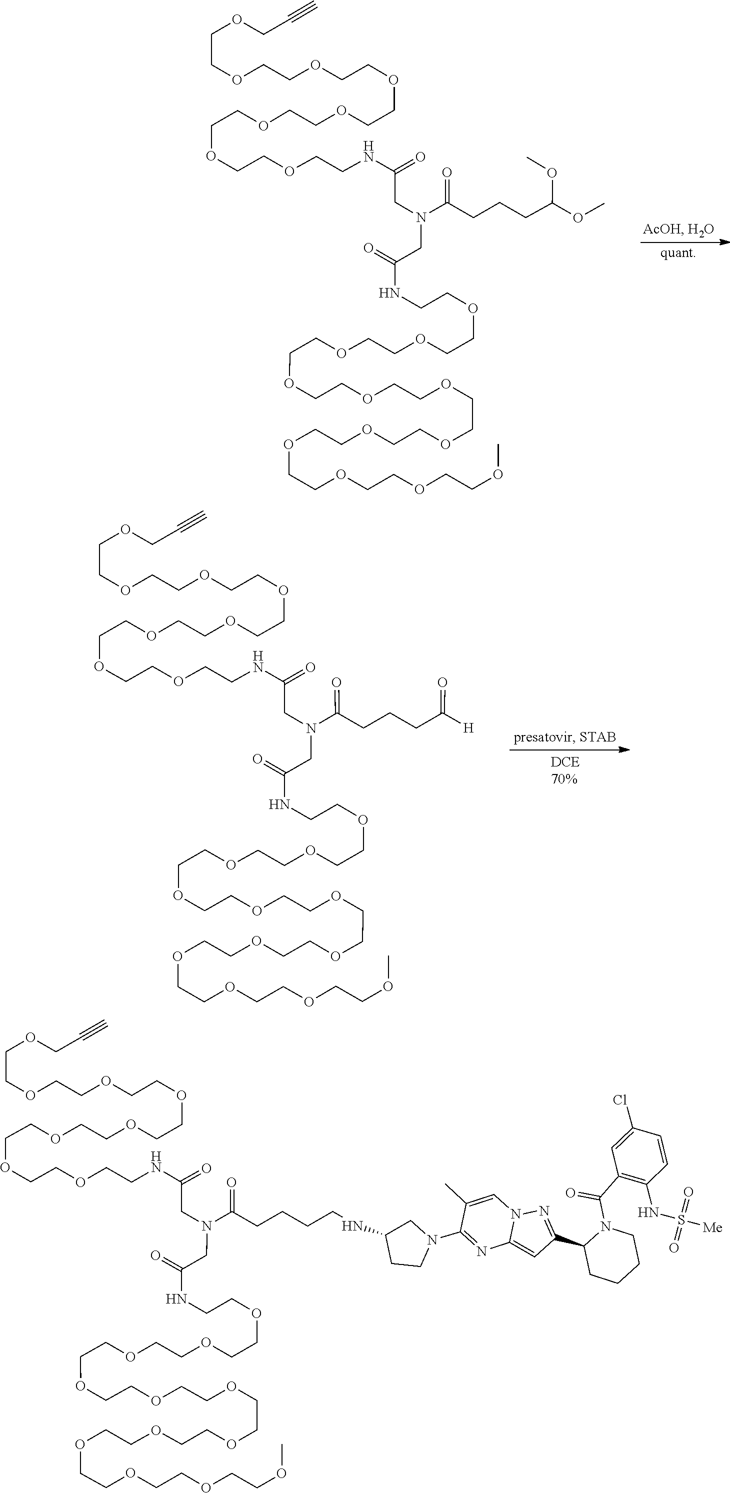 [SMILES: C#CCOCCOCCOCCOCCOCCOCCOCCOCCNC(=O)CN(CC(=O)NCCOCCOCCOCCOCCOCCOCCOCCOCCOCCOCCOCCOC)C(=O)CCCC(OC)OC ; C#CCOCCOCCOCCOCCOCCOCCOCCOCCNC(=O)CN(CC(=O)NCCOCCOCCOCCOCCOCCOCCOCCOCCOCCOCCOCCOC)C(=O)CCCC=O ; C#CCOCCOCCOCCOCCOCCOCCOCCOCCNC(=O)CN(CC(=O)NCCOCCOCCOCCOCCOCCOCCOCCOCCOCCOCCOCCOC)C(=O)CCCCN[C@H]1CCN(c2nc3cc([C@@H]4CCCCN4C(=O)c4cc(Cl)ccc4NS(C)(=O)=O)nn3cc2C)C1]

Step a.

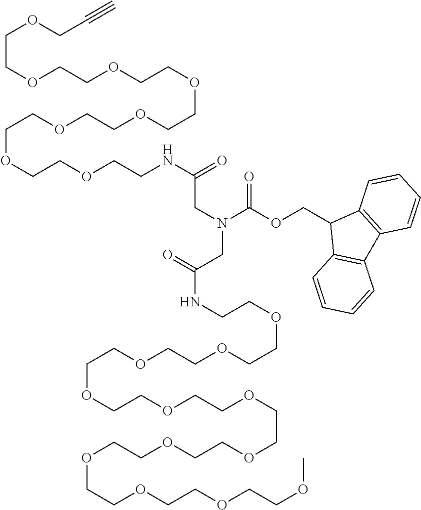 [SMILES: C#CCOCCOCCOCCOCCOCCOCCOCCOCCNC(=O)CN(CC(=O)NCCOCCOCCOCCOCCOCCOCCOCCOCCOCCOCCOCCOC)C(=O)OCC1c2ccccc2-c2ccccc21]

To a 0° C. stirring solution of Fmoc-N-(tert-butyloxycarbonylmethyl)-glycine (672 mg, 1.636 mmol), propargyl-PEG8-amine (667 mg, 1.636 mmol) and DIPEA (712 uL, 4.090 mmol) in DMF (4.0 mL) and DCM (4.0 mL), was added HATU (634 mg, 1.668 mmol). The temperature was raised to ambient and stirring was continued until complete as determined by LCMS. All the volatiles were removed per rotatory evaporation. The resulting residue was purified by RP-C18 column using an Isco COMBIFLASH® liquid chromatography eluted with 0% to 100% water and methanol. The fractions containing the desired product were evaporated per vacuum techniques, affording 1.44 g of a material containing the desired product, but not in high purity (main observed ion found by LCMS: [(M+H)]⁺=801.2).

This material was dissolved in TFA (5.0 mL). LCMS analysis after 2 h showed full conversion of the t-butyl ester to the corresponding acid [(M+H)]⁺=745.2. All the volatiles were evaporated per rotatory evaporation and the residue was taken up in DMF (5.0 mL) and DCM (5.0 mL). This solution was cooled to 0° C., where methyl-PEG12-amine (1.007 g, 1.799 mmol), DIPEA (2.507 mL, 14.39 mmol), and HATU (698 mg, 1.835 mmol) were added. Upon reaction completion as determined by LCMS, all the volatiles were removed via rotatory evaporation. The residue was purified by RP-C18 column using an Isco COMBIFLASH® liquid chromatography eluted with 0% to 100% water and methanol. Yield 0.915 g, 39%. Ions found by LCMS: [(M+2H)/2]⁺=643.8.

Step b.

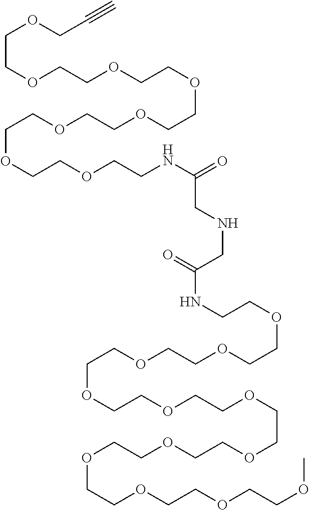 [SMILES: C#CCOCCOCCOCCOCCOCCOCCOCCOCCNC(=O)CNCC(=O)NCCOCCOCCOCCOCCOCCOCCOCCOCCOCCOCCOCCOC]

The product from step a (915 mg, 0.711 mmol) was dissolved in 5% piperidine in DMF (5 mL). after 10 minutes, all the volatiles were removed per rotatory evaporation. The residue was purified by RP-C18 column using an Isco COMBIFLASH® liquid chromatography eluted with 0% to 100% water and methanol. Yield 649 mg, 86%. Ions found by LCMS: [(M+2H)/2]+=532.8.

Step c.

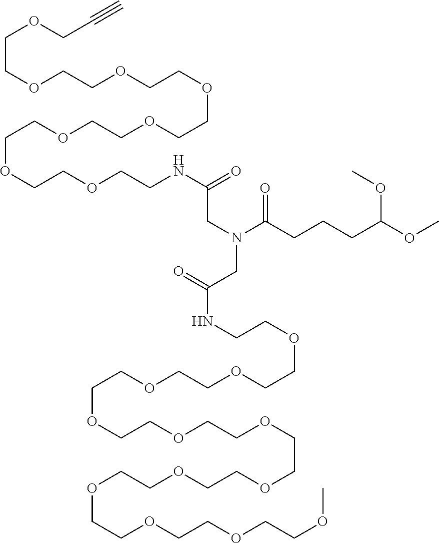 [SMILES: C#CCOCCOCCOCCOCCOCCOCCOCCOCCNC(=O)CN(CC(=O)NCCOCCOCCOCCOCCOCCOCCOCCOCCOCCOCCOCCOC)C(=O)CCCC(OC)OC]

To a 0° C. stirring solution of the product from step b (325 mg, 0.305 mmol), 5,5-dimethoxyvaleric acid (54 mg, 0.336 mmol) and DIPEA (160 uL, 0.916 mmol) in DMF (2.0 mL) and DCM (2.0 mL), was added HATU (118 mg, 0.311 mmol). The temperature was raised to ambient and stirring was continued overnight. All the volatiles were removed per rotatory evaporation. The resulting residue was purified by RP-C18 column using an Isco COMBIFLASH® liquid chromatography eluted with 0% to 100% water and methanol. Yield 305 mg, 83%. Ions found by LCMS: [((M+2H)−MeOH)/2]⁺=588.8.

Step d.

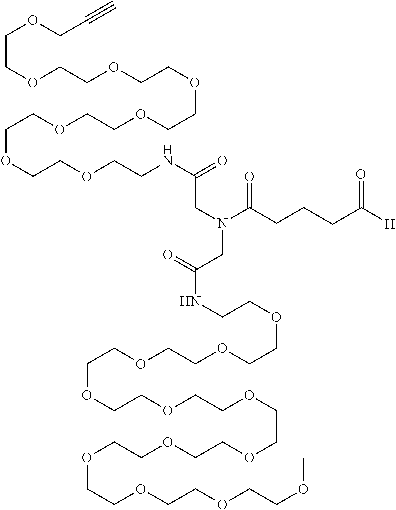 [SMILES: C#CCOCCOCCOCCOCCOCCOCCOCCOCCNC(=O)CN(CC(=O)NCCOCCOCCOCCOCCOCCOCCOCCOCCOCCOCCOCCOC)C(=O)CCCC=O]

The product from step c of this example (300 mg, 0.248 mmol) was dissolved in acetic acid (4.0 mL) and water (2.0 mL), and stirred until full conversion to the desired product was observed by analytical HPLC. All the volatiles were evaporated per rotatory evaporation. This material was used in the next step without further purification. Yield 290 mg, quant.

Step e.

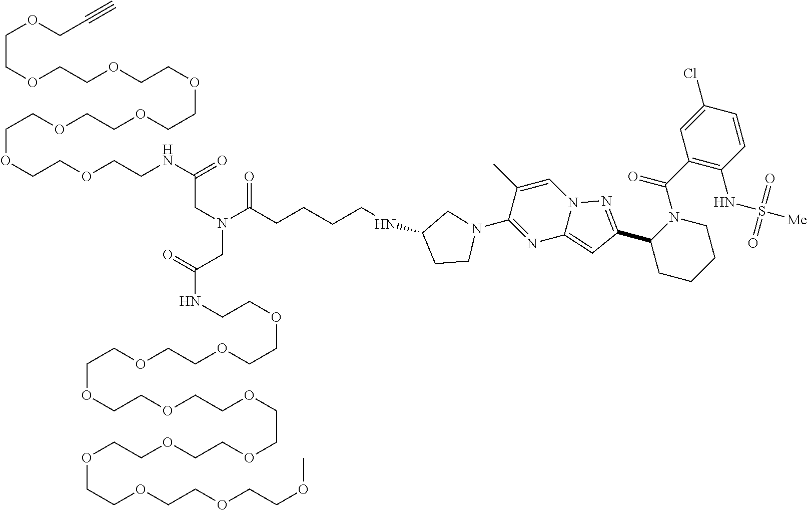

To a stirring solution of the product from step d (109 mg, 0.094 mmol) and presatovir (50 mg, 0.094 mmol) in 1,2-dichloroethane (3.0 mL), was added sodium triacetoxyborohydride (30 mg, 0.141 mmol), while stirring overnight. All the volatiles were removed per rotatory evaporation. The residue was purified by RP-C18 column using an Isco COMBIFLASH® liquid chromatography eluted with 0% to 100% water and methanol. Yield 115 mg, 73%. Ions found by LCMS: [(M+2H)/2]+=839.4.

### Example 26. Synthesis of Int-27

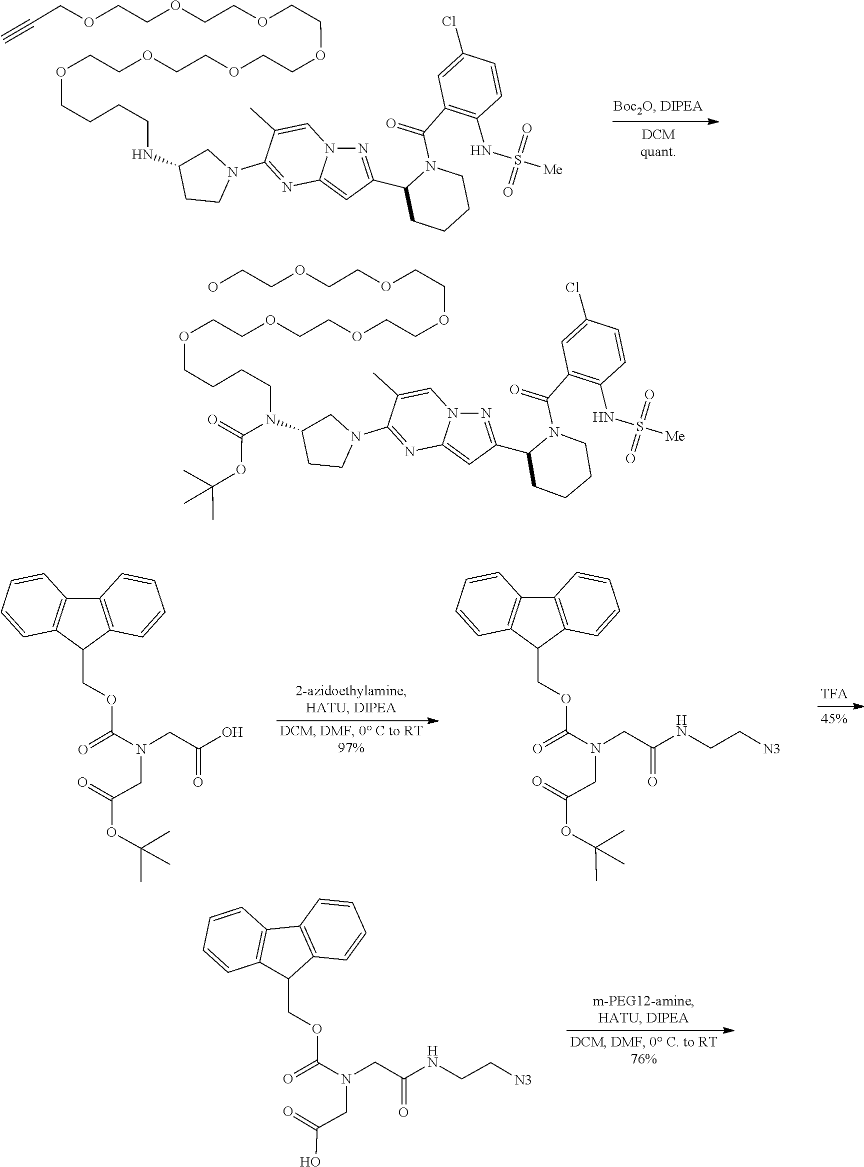

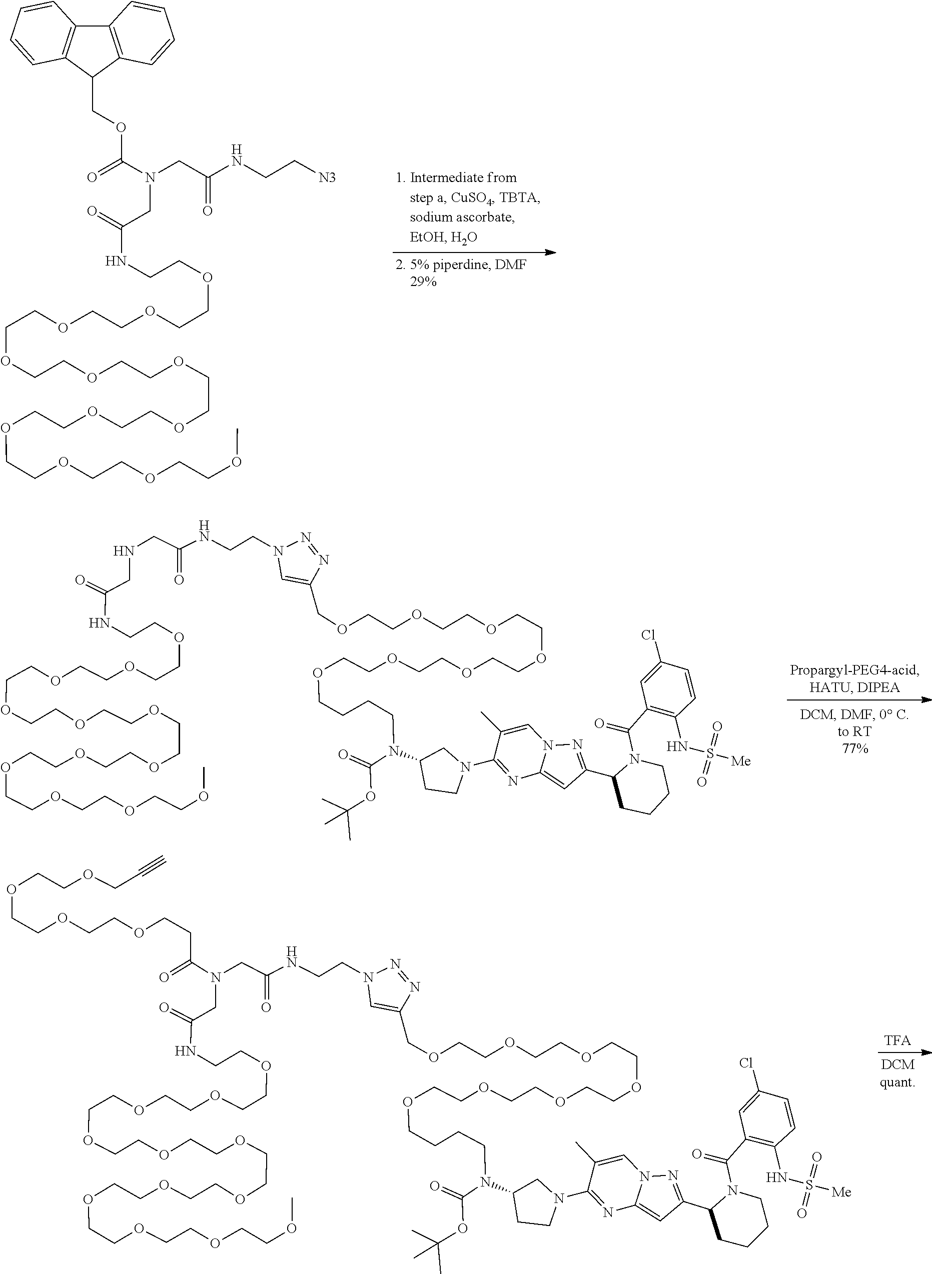 [SMILES: COCCOCCOCCOCCOCCOCCOCCOCCOCCOCCOCCOCCNC(=O)CN(CC(=O)NCCN=[N+]=[N-])C(=O)OCC1c2ccccc2-c2ccccc21 ; COCCOCCOCCOCCOCCOCCOCCOCCOCCOCCOCCOCCNC(=O)CNCC(=O)NCCn1cc(COCCOCCOCCOCCOCCOCCOCCCCN(C(=O)OC(C)(C)C)[C@H]2CCN(c3nc4cc([C@@H]5CCCCN5C(=O)c5cc(Cl)ccc5NS(C)(=O)=O)nn4cc3C)C2)nn1 ; C#CCOCCOCCOCCOCCC(=O)N(CC(=O)NCCOCCOCCOCCOCCOCCOCCOCCOCCOCCOCCOCCOC)CC(=O)NCCn1cc(COCCOCCOCCOCCOCCOCCOCCCCN(C(=O)OC(C)(C)C)[C@H]2CCN(c3nc4cc([C@@H]5CCCCN5C(=O)c5cc(Cl)ccc5NS(C)(=O)=O)nn4cc3C)C2)nn1]

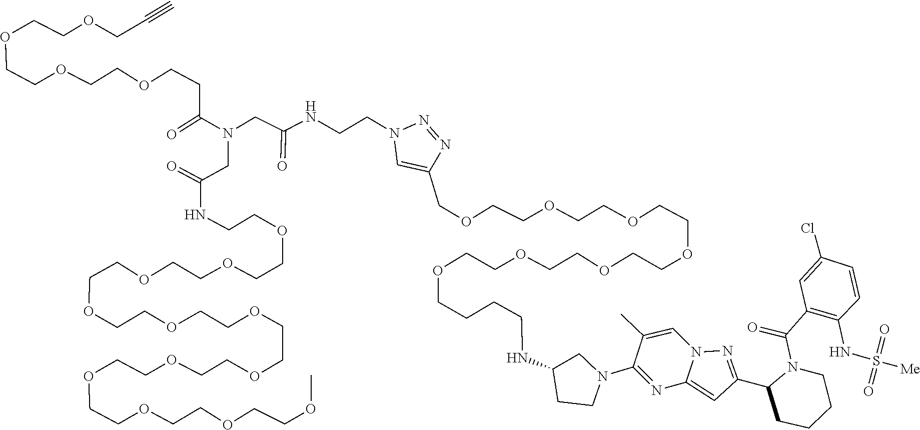

Step a.

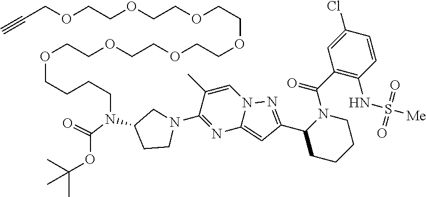 [SMILES: C#CCOCCOCCOCCOCCOCCOCCOCCCCN(C(=O)OC(C)(C)C)[C@H]1CCN(c2nc3cc([C@@H]4CCCCN4C(=O)c4cc(Cl)ccc4NS(C)(=O)=O)nn3cc2C)C1]

To a stirring solution of PEG-presatovir (200 mg, 0.221 mmol, described in Example 8, Int-5) and DIPEA (162 uL, 0.926 mmol) in dichloromethane (3.0 mL), was added di-tert-butyl dicarbonate (72 mg, 0.331 mmol). Stirring was continued until the reaction was complete as determined by HPLC, while gas evolution was allowed through a bubbler. All the volatiles were evaporated per rotatory evaporation. This material was used in the next step without further purification. Yield 224 mg, quant. Ions found by LCMS: [((M+2H)+Na)/2]⁺=514.9; [((M+2H)−Boc)/2]⁺=453.7.

Step b.

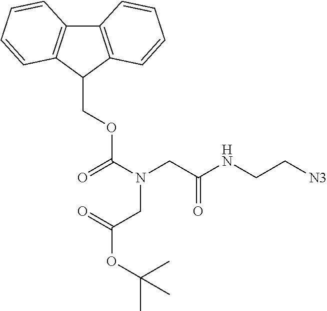 [SMILES: CC(C)(C)OC(=O)CN(CC(=O)NCCN=[N+]=[N-])C(=O)OCC1c2ccccc2-c2ccccc21]

To a 0° C. stirring solution of Fmoc-N-(tert-butyloxycarbonylmethyl)-glycine (2.00 g, 4.861 mmol), 2-azidoethylamine hydrochloride (626 mg, 5.104 mmol) and DIPEA (3.387 mL, 19.44 mmol) in DMF (10 mL) and DCM (10 mL), was added HATU (1.885 g, 4.958 mmol). The temperature was raised to ambient and stirring was continued until complete as determined by LCMS. All the volatiles were removed per rotatory evaporation. The residue was purified by silica column using an Isco COMBIFLASH® liquid chromatography eluted with 0% to 25% methanol in dichloromethane. Yield 2.260 g, 97% yield. Ions found by LCMS: Ions found by LCMS: [(M+H)−tBu]⁺=424.2.

Step c.

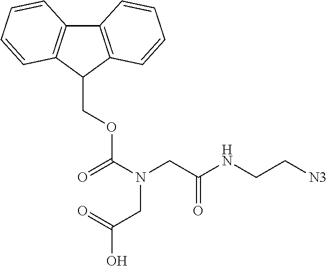 [SMILES: [N-]=[N+]=NCCNC(=O)CN(CC(=O)O)C(=O)OCC1c2ccccc2-c2ccccc21]

The product from step b (2.260 g, 4.713 mmol) was taken up in TFA (10 mL), and stirred until full conversion to product was observed by HPLC. All the volatiles were removed per rotatory evaporation. The resulting residue was purified by silica column using an Isco COMBIFLASH® liquid chromatography eluted with 0% to 20% methanol in dichloromethane, using 1% acetic acid as a modifier. Yield 990 mg, 45% yield. Ions found by LCMS: Ions found by LCMS: [(M+H)+Na]=446.2. [(M+H)]⁺=424.2.

Step d.

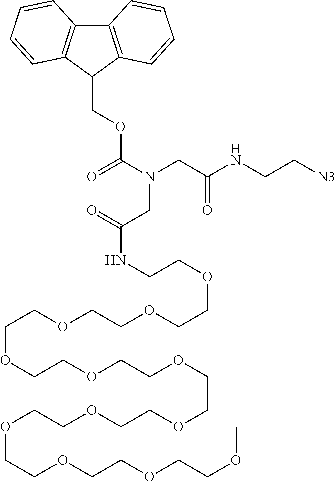 [SMILES: COCCOCCOCCOCCOCCOCCOCCOCCOCCOCCOCCOCCNC(=O)CN(CC(=O)NCCN=[N+]=[N-])C(=O)OCC1c2ccccc2-c2ccccc21]

To a 0° C. stirring solution of the product from step c (990 mg, 2.338 mmol), methyl-PEG12-amine (1.335 g, 2.385 mmol) and DIPEA (1.018 mL, 5.845 mmol) in DMF (5.0 mL) and DCM (5.0 mL), was added HATU (907 mg, 2.385 mmol). The temperature was raised to ambient and stirring was continued until complete by HPLC. All the volatiles were removed per rotatory evaporation. The residue was purified by silica column using an Isco COMBIFLASH® liquid chromatography eluted with 0% to 15% methanol in dichloromethane. Yield 1.704 g, 76% yield. Ions found by LCMS: Ions found by LCMS: [(M+H)]⁺=965.2; [(M+2H)/2]⁺=483.2.

Step e.

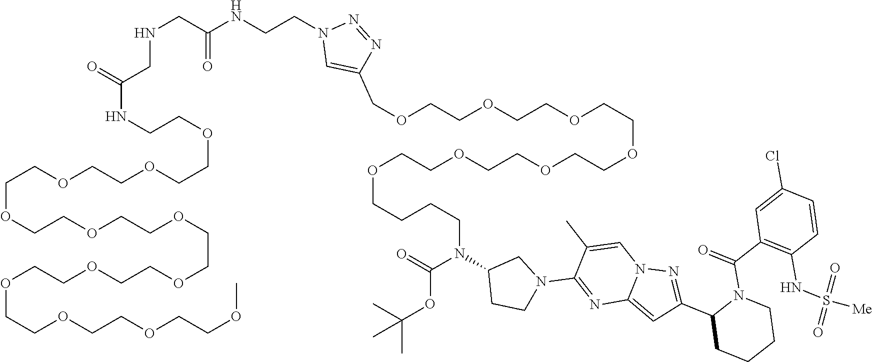 [SMILES: COCCOCCOCCOCCOCCOCCOCCOCCOCCOCCOCCOCCNC(=O)CNCC(=O)NCCn1cc(COCCOCCOCCOCCOCCOCCOCCCCN(C(=O)OC(C)(C)C)[C@H]2CCN(c3nc4cc([C@@H]5CCCCN5C(=O)c5cc(Cl)ccc5NS(C)(=O)=O)nn4cc3C)C2)nn1]

To a stirring solution of the product from step a (222 mg, 0.221 mmol), the product from step d (213 mg, 0.221 mmol), TBTA (12 mg, 0.022 mmol) and cupric sulfate (4 mg, 0.023 mmol) in ethanol (4.0 mL) and water (2.0 mL), was added sodium ascorbate (22 mg, 0.110 mmol). The desired product was formed as confirmed by LCMS [((M+2H)+Na)/2]+=996.9, [(M+2H)/2]+=986.0, [((M+3H)+Na)/3]+=665.2, [(M+3H)/3]+=657.8. Upon completion, copper scavenger SiliaMetS TAAcONa (200 mg, loading 0.45 mmol/g) was added and stirring was continued overnight. The mixture was filtered with the aid of ethanol and to the resulting solution (about 30 mL). The filtrate was concentrated per rotatory evaporation and the residue was taken up in 5% piperidine in DMF (7.0 mL). Upon reaction completion, all the volatiles were removed per rotatory evaporation. The residue was purified by RP-C18 column using an Isco COMBIFLASH® liquid chromatography eluted with 0% to 100% water and methanol. Yield 112 mg, 29%. Ions found by LCMS: [((M+2H)+Na)/2]+=885.8, [(M+2H)/2]+=874.8, [(M+3H)/3]+=583.8.

Step f.

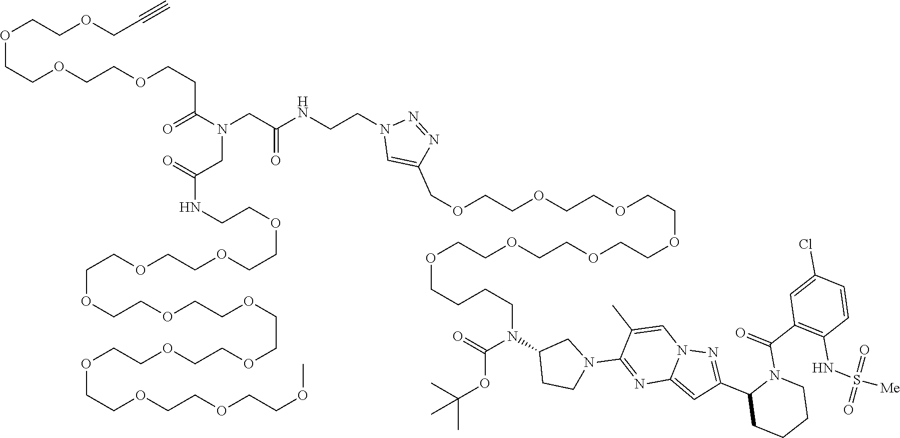 [SMILES: C#CCOCCOCCOCCOCCC(=O)N(CC(=O)NCCOCCOCCOCCOCCOCCOCCOCCOCCOCCOCCOCCOC)CC(=O)NCCn1cc(COCCOCCOCCOCCOCCOCCOCCCCN(C(=O)OC(C)(C)C)[C@H]2CCN(c3nc4cc([C@@H]5CCCCN5C(=O)c5cc(Cl)ccc5NS(C)(=O)=O)nn4cc3C)C2)nn1]

To a 0° C. stirring solution of the product from step e (112 mg, 0.064 mmol), propargyl-PEG4-acid (17 mg, 0.064 mmol) and DIPEA (33 uL, 0.192 mmol) in DMF (3.0 mL) and DCM (0.5 mL), was added HATU (25 mg, 0.065 mmol). The temperature of the reaction was raised to ambient and stirring was continued until complete by HPLC. All the volatiles were removed per rotatory evaporation. The residue was purified by RP-C18 column using an Isco COMBIFLASH® liquid chromatography eluted with 0% to 100% water and methanol. Yield 98 mg, 77%. Ions found by LCMS: [(M+2H)/2]+=996.4, [((M+3H)+2Na)/3]+=679.2, [((M+3H)+Na)/3]+=671.8, [(M+3H)/3]+=664.6.

Step g.

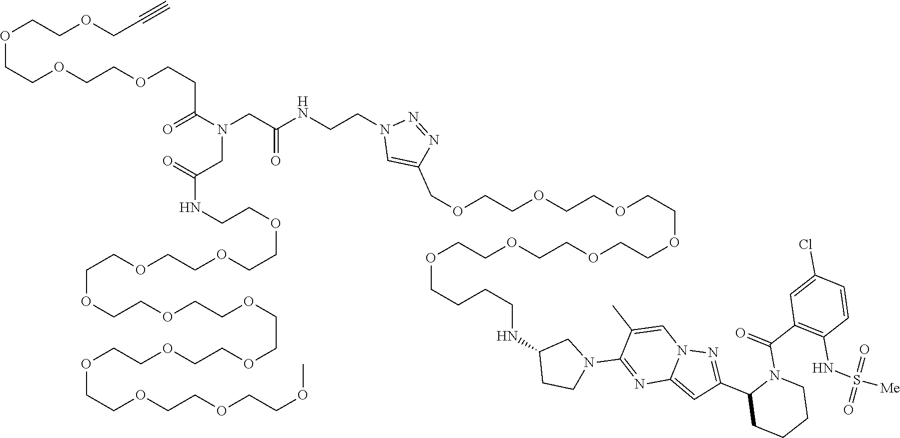 [SMILES: C#CCOCCOCCOCCOCCC(=O)N(CC(=O)NCCOCCOCCOCCOCCOCCOCCOCCOCCOCCOCCOCCOC)CC(=O)NCCn1cc(COCCOCCOCCOCCOCCOCCOCCCCN[C@H]2CCN(c3nc4cc([C@@H]5CCCCN5C(=O)c5cc(Cl)ccc5NS(C)(=O)=O)nn4cc3C)C2)nn1]

To a 0° C. stirring solution of the product from step f (98 mg, 0.049 mmol) in dichloromethane (4.0 mL), was added TFA (2.0 mL). Stirring was continued until full conversion of the starting material to product was observed by HPLC. All of the volatiles were removed per rotatory evaporation. The residue was purified by RP-C18 column using an Isco COMBIFLASH® liquid chromatography eluted with 0% to 100% water and methanol, using 0.1% TFA as a modifier. Yield 100 mg, quant. Ions found by LCMS: [((M+2H)+Na)/2]+=957.0, [(M+2H)/2]+=946.4, [(M+3H)/3]+=631.0.

### Example 27. Synthesis of Conjugate 9

A water solution of L-ascorbic acid sodium salt (4.685 mL, 100 mM in water), was added to a 50 mL centrifuge tube containing a solution of PEG4-azido functionalized Fc (100 mg, 10 mL of a pH 7.4 PBS buffer, 1.874 umol, Example 13; SEQ ID NO: 35 functionalized with PEG4-azide), alkyne functionalized small molecule (75.2 mg, 37.48 umol, described in Example 26, Int-27), 2-aminoguanidine hydrochloride (4.685 mL, 100 mM in water), THPTA (0.937 mL, 50 mM in water), and copper (II) sulfate (0.468 mL, 20 mM in water) in pH 8.5 EPPS buffer (40 mL, 10 mM). The resulting mixture was gently rotated overnight. It was then purified by affinity chromatography over a protein A column, followed size exclusion chromatography (See conjugate purification protocol). Maldi TOF analysis of the purified final product gave an average mass of 69,887 Da (DAR=7.6). Yield 75.2 mg, 75% yield.

### Example 28. Synthesis of Int-28

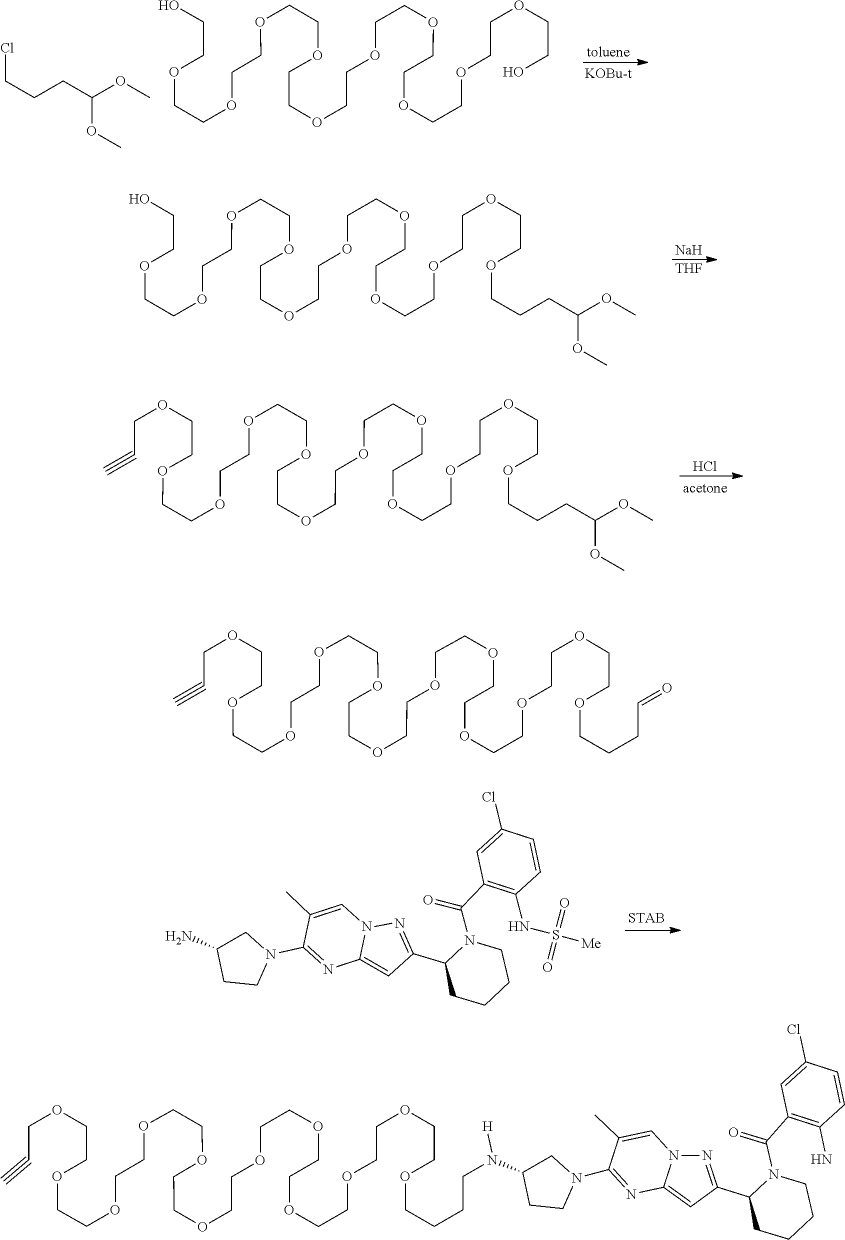

The title compound was prepared analogously to Example 8, Int-5, where the hexaethylene glycol was substituted with PEG12 glycol in step a of the sequence. Ion(s) found by LCMS: [M/2]+1=563.8.

### Example 29. Synthesis of Int-29

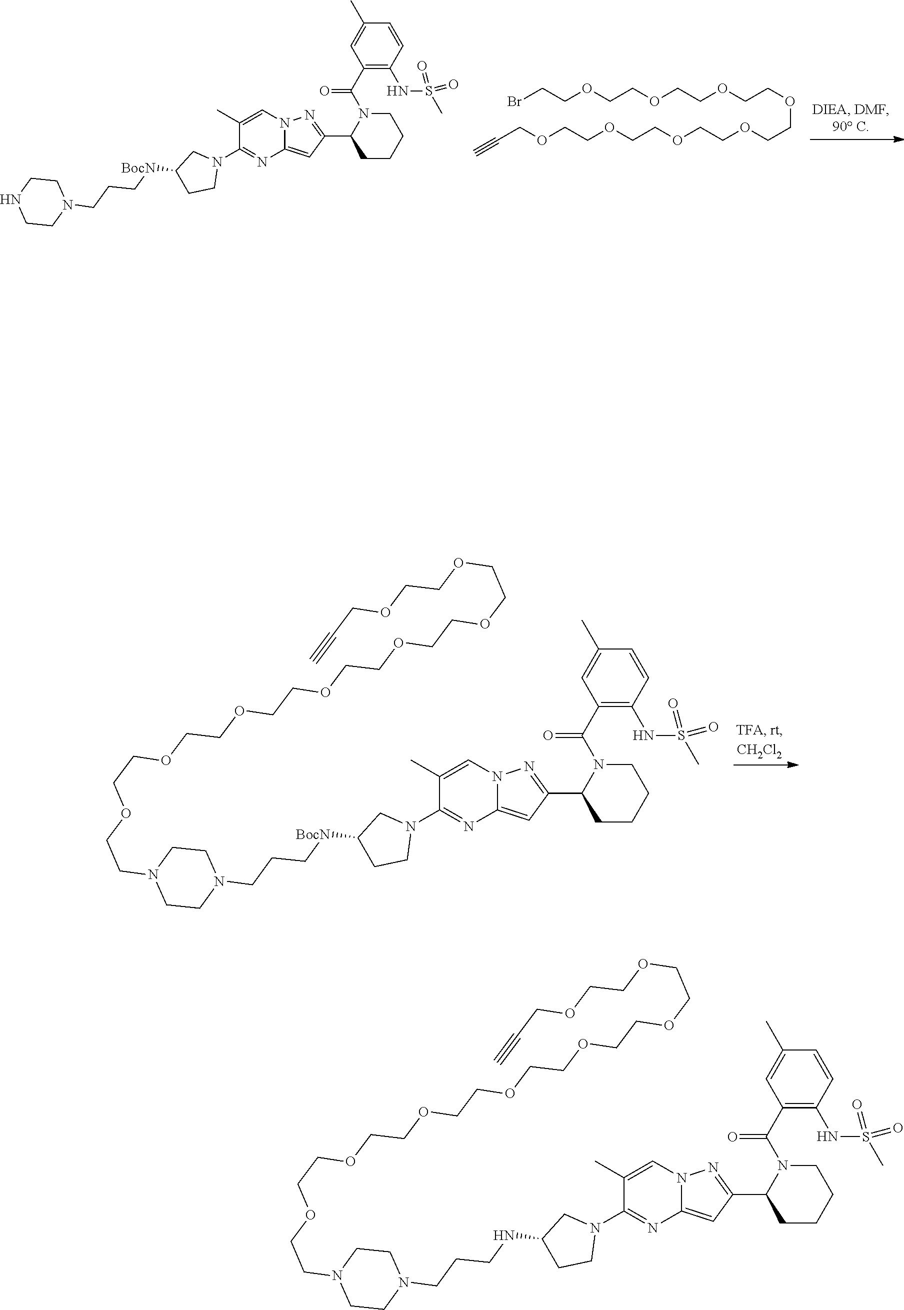 [SMILES: Cc1ccc(NS(C)(=O)=O)c(C(=O)N2CCCC[C@H]2c2cc3nc(N4CC[C@H](N(CCCN5CCNCC5)C(=O)OC(C)(C)C)C4)c(C)cn3n2)c1 ; C#CCOCCOCCOCCOCCOCCOCCOCCOCCBr ; C#CCOCCOCCOCCOCCOCCOCCOCCOCCN1CCN(CCCN(C(=O)OC(C)(C)C)[C@H]2CCN(c3nc4cc([C@@H]5CCCCN5C(=O)c5cc(C)ccc5NS(C)(=O)=O)nn4cc3C)C2)CC1 ; C#CCOCCOCCOCCOCCOCCOCCOCCOCCN1CCN(CCCN[C@H]2CCN(c3nc4cc([C@@H]5CCCCN5C(=O)c5cc(C)ccc5NS(C)(=O)=O)nn4cc3C)C2)CC1]

Step a.

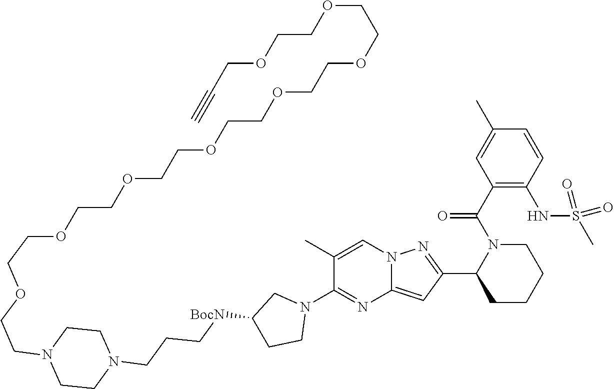 [SMILES: C#CCOCCOCCOCCOCCOCCOCCOCCOCCN1CCN(CCCN(C(=O)OC(C)(C)C)[C@H]2CCN(c3nc4cc([C@@H]5CCCCN5C(=O)c5cc(C)ccc5NS(C)(=O)=O)nn4cc3C)C2)CC1]

To a solution of amine TFA salt (0.25 g, 0.28 mmol, described in Example 19, Int-10) and propagyl-PEG-8-bromide (0.21 g, 0.43 mmol) in anhydrous DMF (2.8 mL) was added DIEA (0.20 mL), 1.13 mmol). The resulting solution was heated at 90° C. for 16 hours. After cooling down to room temperature, the reaction mixture was loaded onto the column directly and purified by reverse phase liquid chromatography (Isco, 10 to 80% acetonitrile and water, using 0.1% TFA as modifier). Yield 0.21 g, 48.8% as TFA salt. Ion found by LCMS: [M+18+H]+=574.8.

Step b.

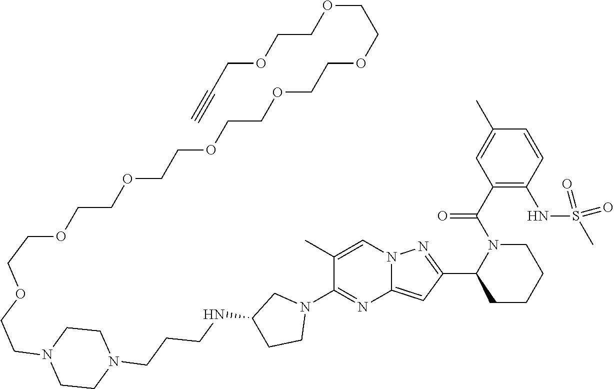 [SMILES: C#CCOCCOCCOCCOCCOCCOCCOCCOCCN1CCN(CCCN[C@H]2CCN(c3nc4cc([C@@H]5CCCCN5C(=O)c5cc(C)ccc5NS(C)(=O)=O)nn4cc3C)C2)CC1]

A solution of Boc-amine product in a previous step (0.21 g, 0.28 mmol) in CH2C12 (3 mL) was added TFA (3 mL). The resulting solution was stirred for 3 hours then concentrated. The residue was purified by reverse phase liquid chromatography (ACCQ, 5 to 50% acetonitrile and water, using 0.1% TFA as modifier). Yield 0.051 g, 22.7% as TFA salt. Ion found by LCMS: [(M+2H)/2]+=524.8.

### Example 30. Synthesis of 5-chloro-2-(chloromethyl)-1-[3-(methanesulfonyl)propyl]-1H-benzimidazole

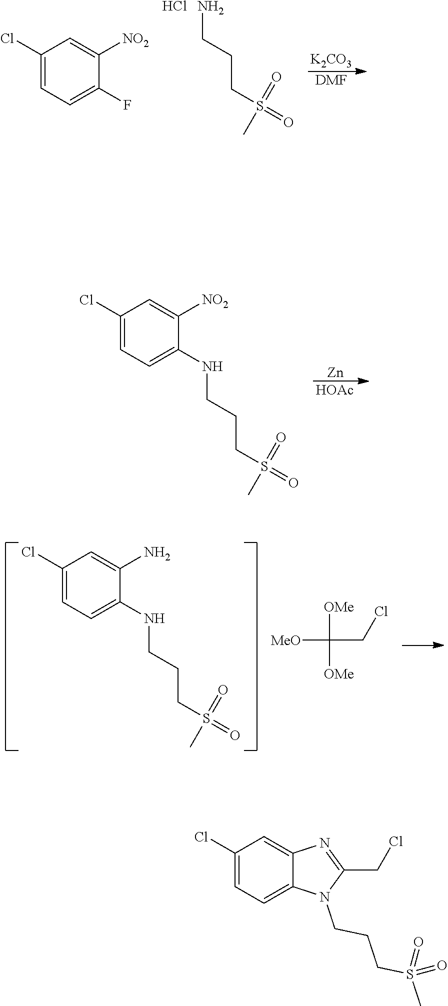

Step a.

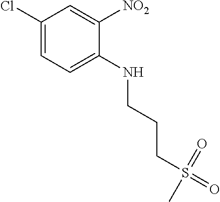 [SMILES: CS(=O)(=O)CCCNc1ccc(Cl)cc1[N+](=O)[O-]]

A solution of 5-Chloro-2-fluoronitrobenzene (0.918 g, 5.23 mmol), in DMF (10 mL) was treated with 3-Methanesulfonyl-propyl-ammonium chloride (1.000 g, 5.757 mmol), and powdered potassium carbonate (2.17 g 15.70 mmol), then heated in a 70 C oil bath. After 12 h LCMS shows complete conversion. The orange colored reaction was filtered to remove potassium carbonate and other insoluble material, concentrated by rotary evaporation, and taken on to the next step without further purification.

Step b.

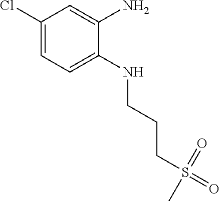 [SMILES: CS(=O)(=O)CCCNc1ccc(Cl)cc1N]

Crude product from the previous step was dissolved in acetic acid (10 mL), heated with a 70° C. oil bath, and treated with zinc powder (1.711 g, 26.17 mmol) in several portions, while rapidly stirring. After 10 minutes the orange color becomes colorless, and LCMS indicated that reaction was complete. The reaction mixture was filtered, giving a green colored solution, which was used immediately in the next step without further purification.

Step c.

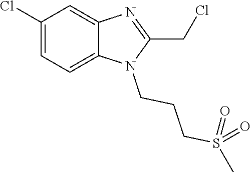 [SMILES: CS(=O)(=O)CCCn1c(CCl)nc2cc(Cl)ccc21]

Crude product from the previous step b, still dissolved in acetic acid, was treated with 2-Chloro-1,1,1-trimethoxyethane (4.85 g, 31.40 mmol), and heated in a 50° C. oil bath. LCMS after 1 hr shows complete conversion to the desired product. All volatiles were removed by rotary evaporation. The remaining oil was purified by flash chromatography (0% to 10% methanol/DCM). Yield 1.32 g, 78.5% yield for three steps. Ion found by LCMS: [M+H]⁺=321.0

### Example 31. Synthesis of 6-chloro-2-(chloromethyl)-3-[3-(methanesulfonyl)propyl]-3H-imidazo[4,5-b]pyridine

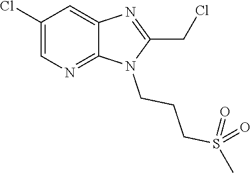 [SMILES: CS(=O)(=O)CCCn1c(CCl)nc2cc(Cl)cnc21]

The title compound was prepared analogously to the compound of Example 30, where 5-chloro-2-fluoro-3-nitropyridine was used in place of 5-Chloro-2-fluoronitrobenzene.

### Example 32. Synthesis of 5-chloro-2-(chloromethyl)-1-[2-(methanesulfonyl)ethyl]-1H-benzimidazole

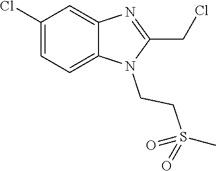 [SMILES: CS(=O)(=O)CCn1c(CCl)nc2cc(Cl)ccc21]

The title compound was prepared analogously to the compound of Example 30, where 2-(methanesulfonyl)ethan-1-amine-hydrogen chloride was used in place of 3-Methanesulfonyl-propyl-ammonium chloride.

### Example 33. Synthesis of 6-chloro-2-(chloromethyl)-3-[2-(methanesulfonyl)ethyl]-3H-imidazo[4,5-b]pyridine

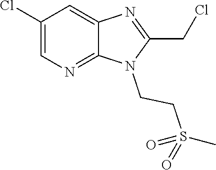 [SMILES: CS(=O)(=O)CCn1c(CCl)nc2cc(Cl)cnc21]

The title compound was prepared analogously to the compound of Example 32, where 5-chloro-2-fluoro-3-nitropyridine was used in place of 5-Chloro-2-fluoronitrobenzene.

### Example 34. Synthesis of 1-butyl-5-chloro-2-(chloromethyl)-1H-benzimidazole

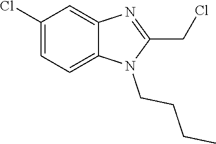 [SMILES: CCCCn1c(CCl)nc2cc(Cl)ccc21]

The title compound was prepared analogously to the compound of Example 30, where 1-butylamine was used in place of 3-Methanesulfonyl-propyl-ammonium chloride.

### Example 35. Synthesis of 3-butyl-6-chloro-2-(chloromethyl)-3H-imidazo[4,5-b]pyridine

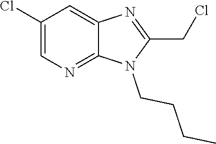 [SMILES: CCCCn1c(CCl)nc2cc(Cl)cnc21]

The title compound was prepared analogously to the compound of Example 34, where 5-chloro-2-fluoro-3-nitropyridine was used in place of 5-Chloro-2-fluoronitrobenzene.

### Example 36. Synthesis of 5-chloro-2-(chloromethyl)-1-(4,7,10,13,16-pentaoxaicos-1-yn-20-yl)-1H-benzimidazole

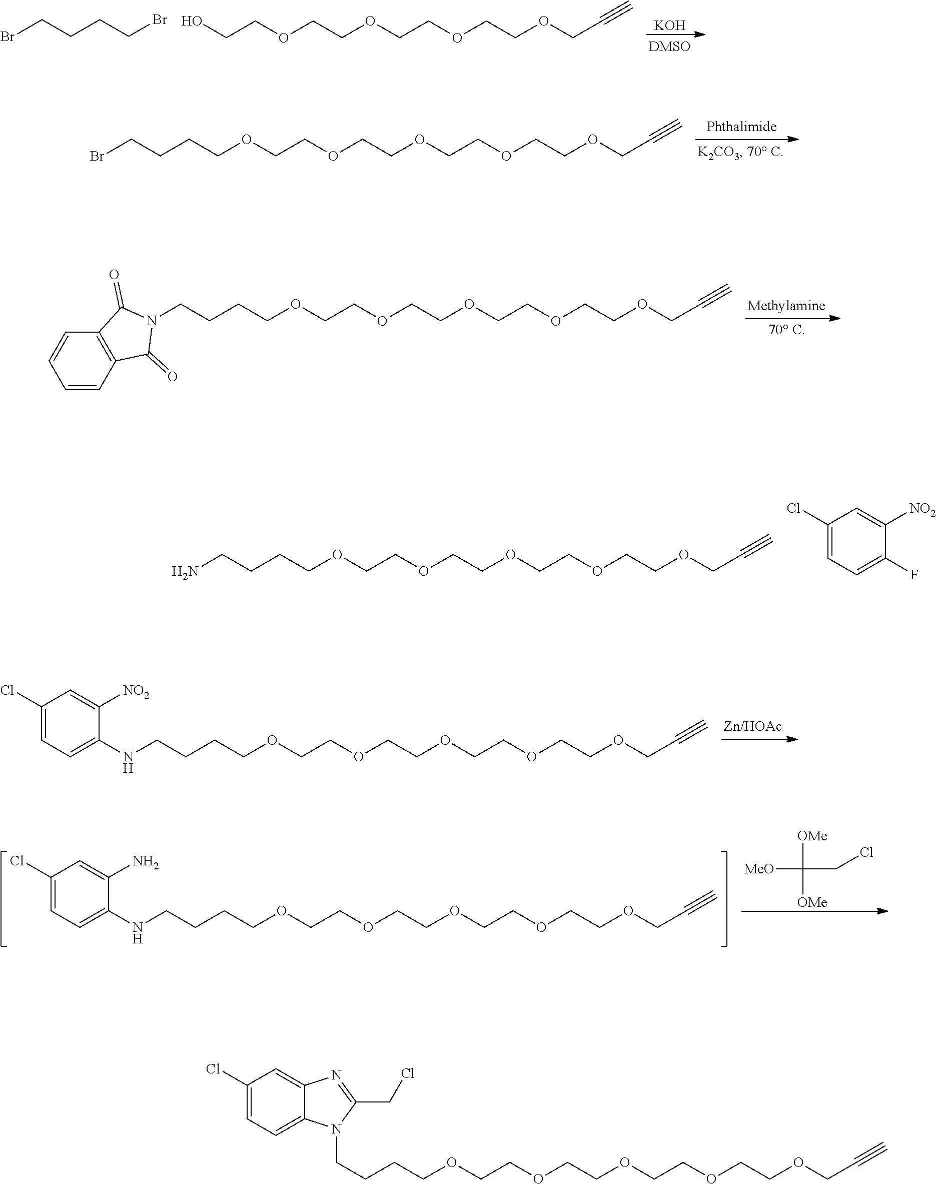

Step a.

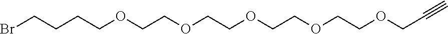 [SMILES: C#CCOCCOCCOCCOCCOCCCCBr]

A stirring solution of propargyl-PEG4-alcohol (2.00 g, 8.61 mmol) and 1,4-dibromobutane (5.57 g, 25.83 mmol), dissolved in DMSO (20 mL), at room temperature, was treated with powdered KOH (0.966 g, 17.22 mmol). The reaction initially became warm and turned dark yellow. After stirring for 1 h, LCMS shows complete consumption of alcohol. The reaction was filtered, diluted with ethylacetate, and extracted with water three times. The water washes were extracted with ethyl acetate three times. The combined ethyl acetate extracts were dried over sodium sulfate, concentrated, and purified by RPLC (10 to 100% ACN/water). Yield 1.10 g, 34.9%.

Step b.

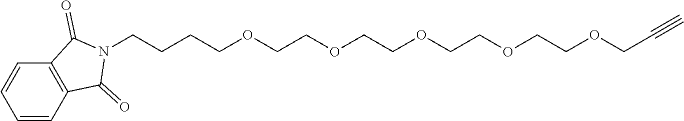 [SMILES: C#CCOCCOCCOCCOCCOCCCCN1C(=O)c2ccccc2C1=O]

To a stirring solution of product from the previous step a (1.100 g, 3.00 mmol) and phthalimide (0.881 g, 6.00 mmol) in DMF (7 mL), was added powdered potassium carbonate (1.66 g, 11.98 mmol). The mixture was stirred in a 70° C. oil bath for 1 h, at which time LCMS showed complete disappearance of starting bromide. The reaction mixture was filtered, concentrated and purified by RPLC (10 to 100% ACN/water). Yield 1.28 g, 96.6% yield. Ion(s) found by LCMS: [M+H]⁺=434.0

Step c.

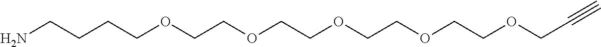 [SMILES: C#CCOCCOCCOCCOCCOCCCCN]

A solution of product from the previous step b (1.10 g, 2.54 mmol) dissolved in ethanol (3 mL), was treated with 40% aqueous methyl amine (3 mL) and heated in 70 C oil bath for 1 h, at which time LCMS show complete consumption of starting material. The reaction was concentrated by rotary evaporation, then stored under high vacuum overnight, and used as mixture of N-methyl-phthalimide and desired product in the next step without further purification.

Step d.

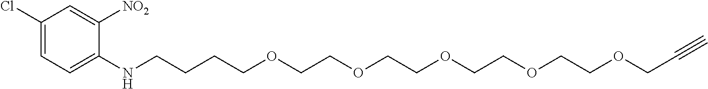 [SMILES: C#CCOCCOCCOCCOCCOCCCCNc1ccc(Cl)cc1[N+](=O)[O-]]

Crude product (2.538 mmol) from the previous step c was dissolved in DMF (5 mL), treated with DIEA (1.81 mL, 4 eq) and 5-Chloro-2-fluoronitrobenzene (0.535 g, 3.046 mmol), and heated in 50° C. oil bath. After stirring overnight LCMS showed complete consumption of amino-PEG starting material. The crude mixture was concentrated and purified by RPLC (10 to 100% ACN/water). Yield 0.62 g, 52% yield for two steps. Ion(s) found by LCMS: [M+H]⁺=459.0

Step e.

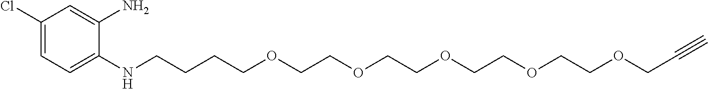 [SMILES: C#CCOCCOCCOCCOCCOCCCCNc1ccc(Cl)cc1N]

Product from the previous step d (0.620 g, 1.35 mmol), was dissolved in acetic acid (4 mL), heated in a 50° C. oil bath, and treated with zinc powder (1.77 g, 27.02 mmol), portion-wise over 15 minutes. After 20 min the reaction changes color from orange to colorless, and LCMS shows complete consumption of starting material. The reaction mixture was filtered to remove zinc powder and used in the next step as a solution in acetic acid.

Step f.

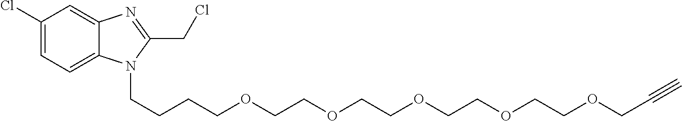 [SMILES: C#CCOCCOCCOCCOCCOCCCCn1c(CCl)nc2cc(Cl)ccc21]

Crude product from the previous step e (1.35 mmol) was heated in a 50° C. oil bath, and treated with 2-chloro-1,1,1-trimethoxyethane (1.25 g, 8.10 mmol). LCMS after 1 hr shows complete consumption of starting material. The reaction was concentrated by rotary evaporation, then purified by flash chromatography (0 to 10% MeOH/DCM). Yield 0.45 g, 68.3% yield for two steps. Ion(s) found by LCMS: [M+H]⁺=486.8.

### Example 37. tert-butyl T-oxo-1′,2′-dihydrospiro[piperidine-4,3′-pyrrolo[2,3-c]pyridine]-1-carboxylate

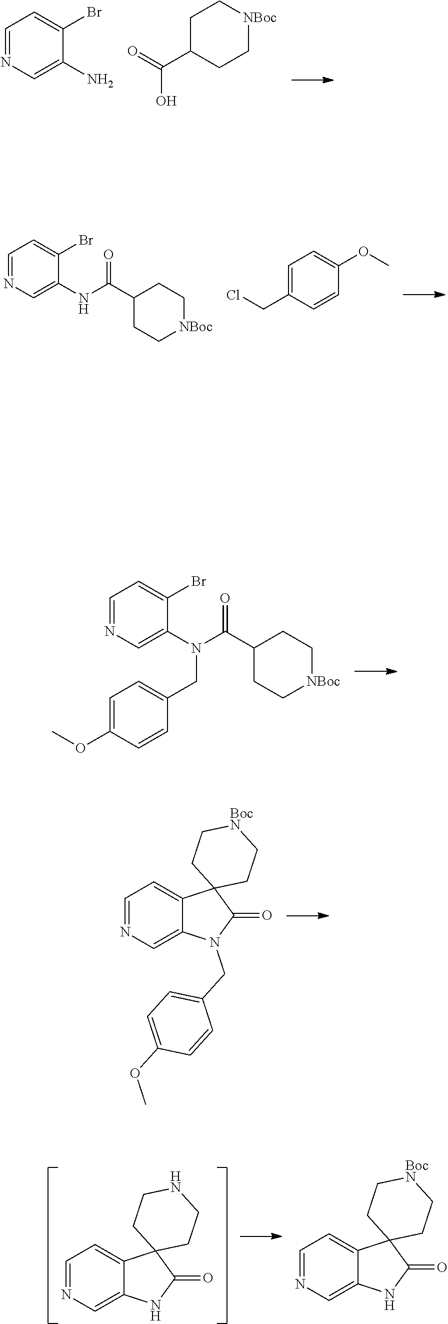

Step a.

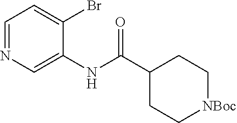 [SMILES: CC(C)(C)OC(=O)N1CCC(C(=O)Nc2cnccc2Br)CC1]

T3P (41.6 mL, 69.9 mmol, 50% by wt. in ethyl acetate) was added, dropwise over 10 minutes, to a stirring mixture of 2-amino-2-bromo-pyridine (11 g, 63.6 mmol), N-Boc-piperazine carboxylic acid (16 g, 69.9 mmol), and DIPEA (16.4 g, 127.2 mmoL) in ethyl acetated (75 mL) cooled to 0° C. The ice bath was removed and the reaction was stirred for 24 hours. The reaction mixture was diluted with water, extracted into ethyl acetate (3×, 25 mL). The combined organic extracts were dried over sodium sulfate, and concentrated on the rotary evaporator. The crude product was by silica gel chromatography on the ISCO COMBI FLASH (15% to 100% ethyl acetate in hexanes, 25 minutes). The pure fractions were pooled and concentrated to afford the intermediate as a light orange oil. Yield 61%. LC/MS [M+H]+=384.2.

Step b.

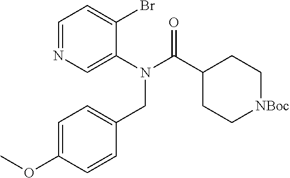 [SMILES: COc1ccc(CN(C(=O)C2CCN(C(=O)OC(C)(C)C)CC2)c2cnccc2Br)cc1]

p-Methoxy benzyl chloride (11.6 g, 74.2 mmol) was added to a mixture of the intermediate from step a. of this example (19.1 g, 49.4 mmol) and cesium carbonate (24.1 g, 74.1 mmol) in DMF (30 mL). The reaction was stirred at room temperature for 12 hours at which time it was diluted with water and extracted into ethyl acetate (3×, 30 ml). The combined organic extracts were washed with brine, dried over sodium sulfate, and concentrated on the rotary evaporator. The crude product was by silica gel chromatography on the ISCO COMBI FLASH (10% to 100% ethyl acetate in hexanes, 25 minutes). The pure fractions were pooled and concentrated to afford the intermediate as a light orange oil. Yield 68%. LC/MS [M+Na]⁺=526.0.

Step c.

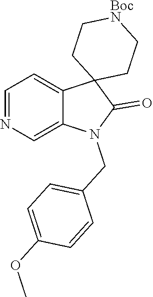 [SMILES: COc1ccc(CN2C(=O)C3(CCN(C(=O)OC(C)(C)C)CC3)c3ccncc32)cc1]

A mixture of intermediate b. (17.0 g, 33.7 mmol), described in this example, palladium(II)acetate (0.76 g, 3.4 mmol), tricyclohexyl phosphine (1.9 g, 6.7 mmol) were dissolved in dioxane (40 mL) in a sealed tube. Nitrogen was gently bubbled though the mixture for 10 minutes at which point sodium t-butoxide (4.9 g, 50.5 mmol) was added and nitrogen was bubbled through the reaction mixture for an additional 10 minutes. The tube was sealed and heated at 120° C. for 16 hours. The mixture was cooled and concentrated on the rotary evaporator. The dark, viscous product mixture was purified by silica gel chromatography on the ISCO COMBI FLASH (0% to 10% methanol in DCM, 25 minutes). The pure fractions were pooled and concentrated to afford the intermediate as a light yellow oil. Yield 84%. LC/MS [M+H]⁺=424.2.

Step d.

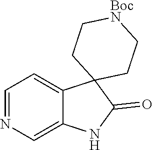 [SMILES: CC(C)(C)OC(=O)N1CCC2(CC1)C(=O)Nc1cnccc12]

The intermediate from step c of this example (3 g, 7.1 mmol) and anisole (3.8 g, 35.4 mmol) were stirred in a solution of 10% triflic acid in TFA (25 mL) at 70° C. for 12 hours (LC/MS [M+H]⁺=204.2). The mixture was cooled and concentrated on the rotary evaporator and azeotroped with toluene (3×). The dark, viscous product mixture was taken up in acetonitrile (50 mL) and cooled to 0° C. The pH was adjusted to 8 by the dropwise addition of DIPEA and boc anhydride (1.5 g, 7.1 mmoL) was added and the reaction was stirred for 40 minutes. The solvent was removed by the rotary evaporator and the crude product mixture was purified by RP HPLC (ISCO COMBI FLASH, 10-95% acetonitrile in DI water, 0.1% TFA, 40 minute gradient). The pure fractions were pooled and lyophilized to afford the product as a white solid. Yield 69%. LC/MS [M+H]⁺=304.2.

### Example 38. Synthesis of propan-2-yl 2′-oxo-1′,2′-dihydrospiro[piperidine-4,3′-pyrrolo[2,3-c]pyridine]-1-carboxylate

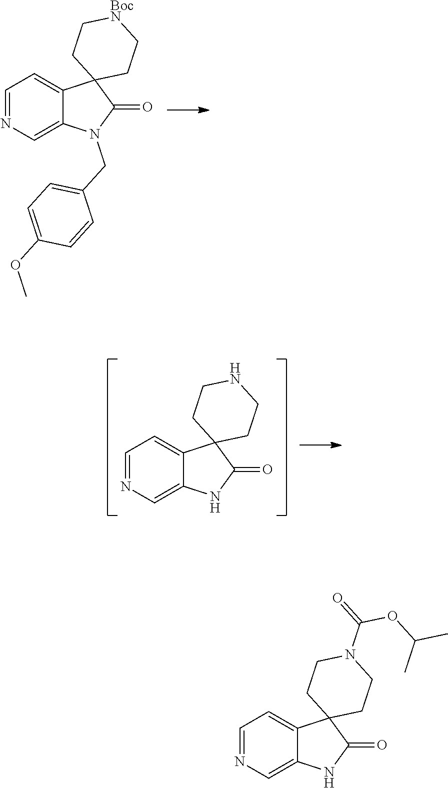

Step a.

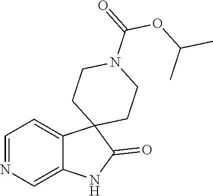 [SMILES: CC(C)OC(=O)N1CCC2(CC1)C(=O)Nc1cnccc12]

The intermediate from step c. of the product of example 36 (0.75 g, 1.8 mmol) and anisole (0.96 g, 8.9 mmol) were stirred in a solution of 10% triflic acid in TFA (10 mL) at 70° C. for 12 hours (LC/MS [M+H]+=204.2). The mixture was cooled and concentrated on the rotary evaporator and azeotroped with toluene (3×). The dark, viscous product mixture was taken up in acetonitrile (20 mL) and cooled to 0° C. The pH was adjusted to 8 by the dropwise addition of DIPEA and isopropyl chloroformate (0.25 g, 2 mmoL) was added and the reaction was stirred for 40 minutes. The solvent was removed by the rotary evaporator and the crude product mixture was purified by RP HPLC (ISCO COMBI FLASH, 10-95% acetonitrile in DI water, 0.1% TFA, 40 minute gradient). The pure fractions were pooled and lyophilized to afford the product as a white solid. Yield 69%. LC/MS [M+H]⁺=290.2.

### Example 39. Synthesis of 5-chloro-2-(chloromethyl)-1-(3,6,9,12-tetraoxapentadec-14-yn-1-yl)-1H-benzimidazole

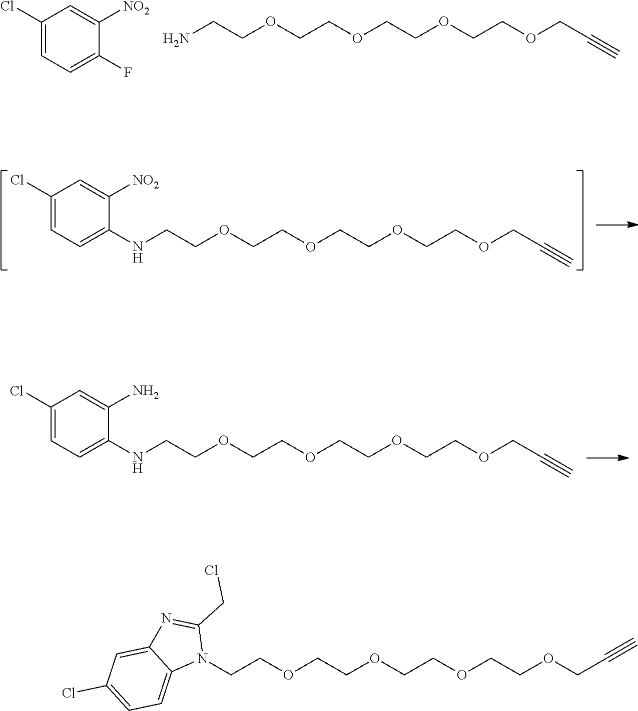

Step a.

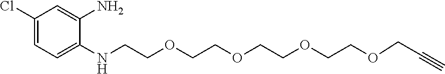 [SMILES: C#CCOCCOCCOCCOCCNc1ccc(Cl)cc1N]

Propargyl peg-4 amine (395 mg, 1.71 mmol) was added to a mixture of 2-fluoro-5-chloro-nitrobenzene (200 mg, 1.39 mmol) and triethylamine (230 mg, 2.28 mmol) in ethanol (10 mL) and the reaction was stirred at 90° C. for 12 hours at which point the starting material had been fully converted to product. The mixture was concentrated and purified by silica gel chromatography (ISCO COMBI FLASH, 0-100% ethyl acetate in hexanes, 25 minute gradient). The pure fractions were pooled and lyophilized to afford the product as a clear oil, (LC/MS[M+H]⁺=387.2). The nitro intermediate was taken up in glacial acetic acid (10 ml) and zinc (50 mg, 0.78 mmol) was added and the mixture was stirred for 1 hour. The reaction mixture was filtered and concentrated on the rotary evaporator. The product was taken forward without further purification. Yield 63%, 2 steps. LC/MS [M+H]⁺=357.2.

Step b.

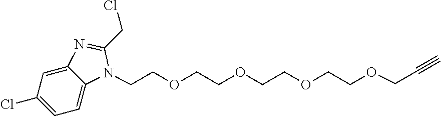 [SMILES: C#CCOCCOCCOCCOCCn1c(CCl)nc2cc(Cl)ccc21]

2-Chloromethyl-1,1,1-trimethoxy ethane (550 mg, 3.56 mmol) was added to the diamine intermediate, described in step a of this example, (254 mg, 0.71 mmol) in glacial acetic acid (5 mL) and the reaction was stirred at 70° C. for 2 hours. The mixture was concentrated on the rotary evaporator and purified by silica gel chromatography (ISCO COMBI FLASH, 0-8% methanol in DCM, 25 minute gradient). The pure fractions were pooled and concentrated to afford the product as a brown oil. Yield 69%. LC/MS [M+H]⁺=414.9.

### Example 40. Synthesis of 3-{2-[4-(3,6,9,12-tetraoxapentadec-14-yn-1-yl)piperazin-1-yl]ethoxy}propanoic acid

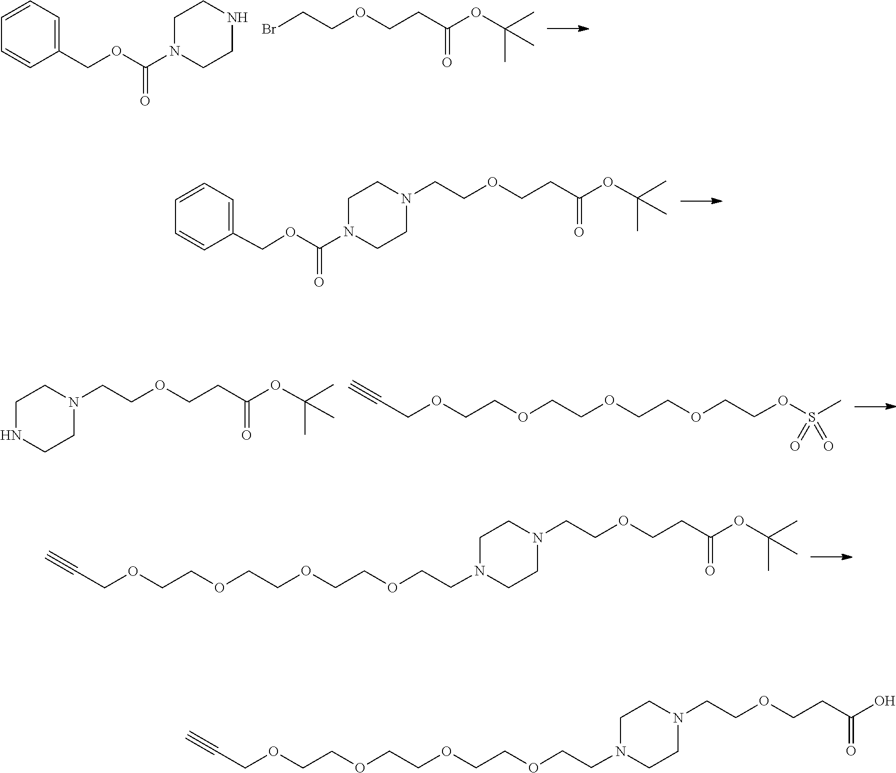

Step a.

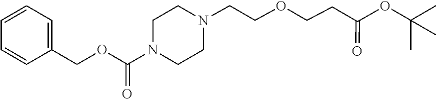 [SMILES: CC(C)(C)OC(=O)CCOCCN1CCN(C(=O)OCc2ccccc2)CC1]

A solution of cbz-piperazine (1.697 g, 7.703 mmol), tert-butyl 3-(2-bromoethoxy)propanoate (1.95 g, 7.703 mmol), and DIEA (2.68 mL, 15.41 mmol), dissolved in acetonitrile (20 mL) were heated in a 75° C. oil bath overnight. The crude mixture was concentrated by rotary evaporation, made slightly acid with TFA, and purified by RPLC (5-100% ACN/water with 0.1% TFA). Yield of mono TFA salt was 3.71 g, 95%. Ion(s) found by LCMS: [M+H]⁺=393.2

Step b.

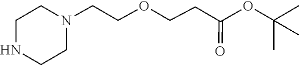 [SMILES: CC(C)(C)OC(=O)CCOCCN1CCNCC1]

A solution product from the previous step a (3.71 g, 7.33 mmol), and 5% Pd/C (1.5 g) was dissolved in methanol (30 mL), and vacuum flushed with hydrogen gas and stirred with hydrogen from a balloon for 1 hr. The reaction was filtered through celite. The filtrate was concentrated by rotary evaporation, and then used in the next step without further purification. Ion(s) found by LCMS: [M+H]⁺=259.2

Step c.

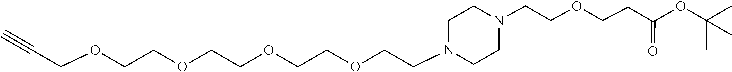 [SMILES: C#CCOCCOCCOCCOCCN1CCN(CCOCCC(=O)OC(C)(C)C)CC1]

Product from the previous step b (2.73 g, 7.33 mmol), and propargyl-PEG4-mesylate (2.50 g, 8.06 mmol), were dissolved in acetonitrile (20 mL), then treated with DIEA (3.831 mL, 22.0 mmol), and heated in a 75° C. oil bath for 24 h. The crude reaction was concentrated by rotary evaporation, made slightly acid with TFA, and purified by RPLC (10-100% ACN/water with 0.1% TFA). Yield 3.24 g, 63.1%. Ion(s) found by LCMS: [M+H]⁺=473.3

Step d.

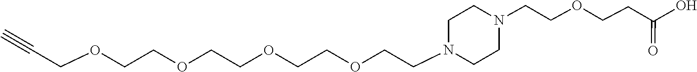 [SMILES: C#CCOCCOCCOCCOCCN1CCN(CCOCCC(=O)O)CC1]

Product from the previous step c (3.24 g, 4.62 mmol), was treated with TFA (15 mL) and stirred for 1 hr. The reaction was stripped of volatiles by rotary evaporation followed by storage under high vacuum for 12 h hr. The crude product was used without further purification. Ion(s) found by LCMS: [M+H]⁺=417.2

### Example 41. Synthesis of 1-[4-(prop-2-yn-1-yl)piperazin-1-yl]-3,6,9,12-tetraoxapentadecan-15-oic acid

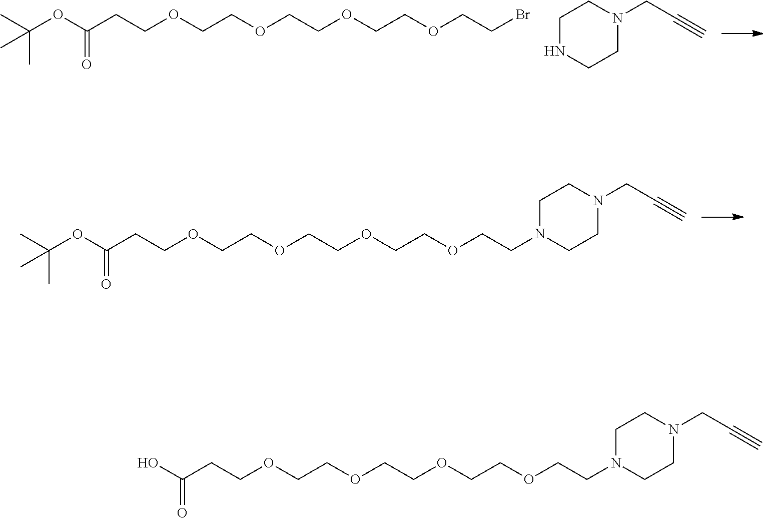

Step a.

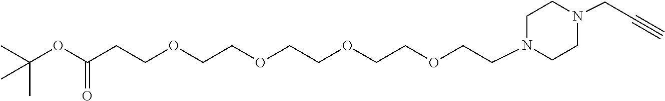 [SMILES: C#CCN1CCN(CCOCCOCCOCCOCCC(=O)OC(C)(C)C)CC1]

A solution of bromo-PEG4-t-butyl ester (1.00 g, 2.60 mmol), propargyl piperazine 8TFA salt (3.20 g, 3.89 mmol), was treated with powdered potassium carbonate (3.59 g, 25.9 mmol), then heated in an oil bath for 12 h at 75° C. The reaction was filtered, concentrated to an oil, and made slightly acidic with TFA, then purified by RPLC (0 to 100% ACN/water with 0.1% TFA). Yield 0.911 g double TFA salt, 53.5%. Ion(s) found by LCMS: [M+H]⁺=429.3

Step b.

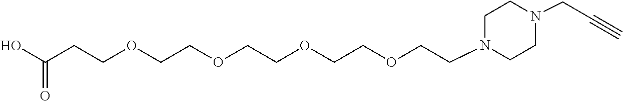 [SMILES: C#CCN1CCN(CCOCCOCCOCCOCCC(=O)O)CC1]

Product from the previous step a (0.911 g, 1.39 mmol) was dissolved in 4M HCL in dioxane and stirred for 1 hr. The reaction was concentrated by rotary evaporation, and stored under high vacuum overnight. Crude yield of 9 HCl salt was 0.92 g, 98%.

### Example 42. Synthesis of Int-39

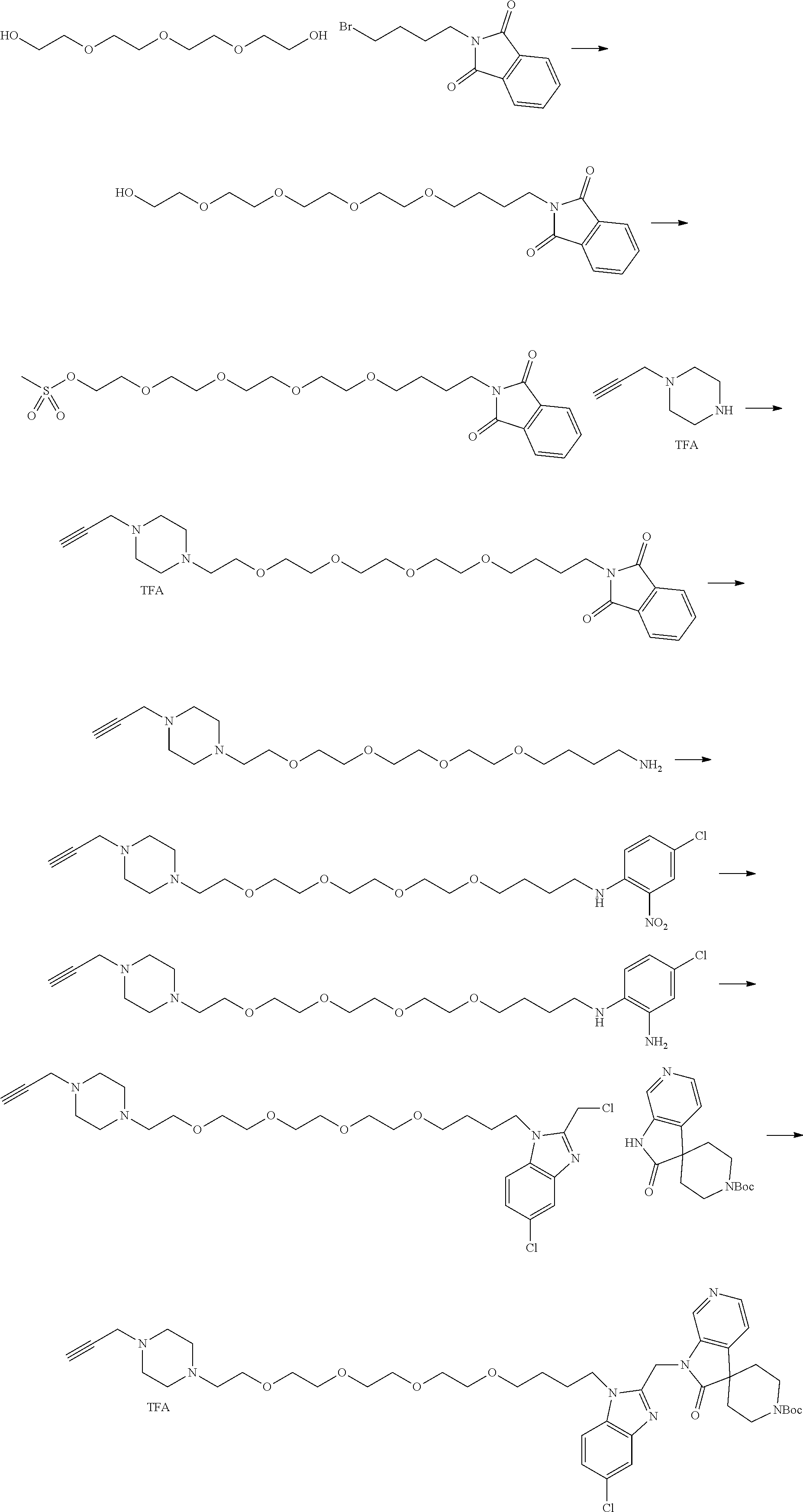

Step a.

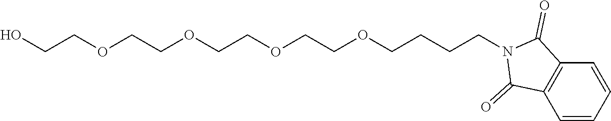 [SMILES: O=C1c2ccccc2C(=O)N1CCCCOCCOCCOCCOCCO]

To a solution of tetraethylene glycol (3.8 g, 20 mmol) in dry DMF was slowly added sodium hydride (0.48 g, 12 mmol, 60% in mineral oil). The solution was stirred on ice bath for 10 mins, and then 2-(4-bromobutyl)isoindoline-1,3-dione (2.8 g, 10 mmol) was added. The reaction solution was stirred at room temperature for 16 hours, quenched with tert-butanol (1 ml) and concentrated. The residue was dissolved in DCM (50 ml) and the solution was washed with water (3×, 10 ml), brine (10 ml), then dried over sodium sulfate, filtered and concentrated. The crude product was purified by silica gel chromatography on an Isco Combi Flash (15% to 100% ethyl acetate in hexanes). Yield 1.32 g, 35%. Ion found by LCMS: [M+H]⁺=396.0.

Step b.

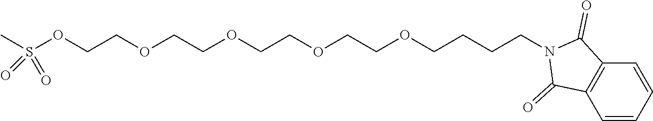 [SMILES: CS(=O)(=O)OCCOCCOCCOCCOCCCCN1C(=O)c2ccccc2C1=O]

To a solution of the product from step a (1 g, 2.5 mmol) in DCM (25 ml) was added triethylamine (0.7 ml, 5 mmol), followed by methanesulfonyl chloride (0.3 ml, 3.75 mmol). The reaction solution was stirred for 2 hours, and then washed with aq HCl (1N, 2×, 5 ml), water (10 ml), brine, and concentrated to give the crude product. Yield 1.12 g, 95%. Ion found by LCMS: [M+H]⁺=474.8.

Step c.

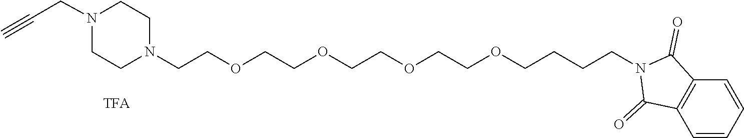 [SMILES: C#CCN1CCN(CCOCCOCCOCCOCCCCN2C(=O)c3ccccc3C2=O)CC1]

A mixture of the product from step b (1.12 g, 2.3 mmol), 1-(prop-2-yn-1-yl)piperazine (2.6 g, 2.5 mmol), and triethylamine (2.7 ml, 20 mmol) in dry DMF were heated at 90° C. for 3 hours. The solution was cooled, concentrated, and purified by RPLC (5% to 50% acetonitrile/water, using 0.1% TFA as modifier). Yield 617 mg, 51% for two steps. Ion found by LCMS: [M+H]⁺=502.3.

Step d.

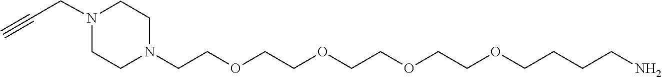 [SMILES: C#CCN1CCN(CCOCCOCCOCCOCCCCN)CC1]

The product from step c (450 mg, 0.9 mmol) was dissolved in concentrated NH₄OH (5 mL of 40% aqueous) and the solution was heated at 50° C. overnight. The solution was cooled, concentrated and the residue was dissolved in water, washed with DCM, concentrated and lyophilized to give the crude product which was used without further purification. Ion found by LCMS: [M+H]⁺=372.2.

Step e.

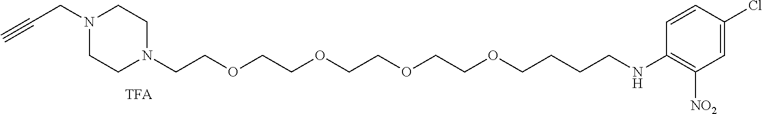 [SMILES: C#CCN1CCN(CCOCCOCCOCCOCCCCNc2ccc(Cl)cc2[N+](=O)[O-])CC1]

A mixture of the product from step d, 5-chloro-2-fluoronitrobenzene (210 mg, 1.2 mmol), and K₂CO₃ (496 mg, 3.6 mmol) in dry acetonitrile was heated at 70° C. for 2 hours. The solution was cooled, filtered, concentrated, and purified by RPLC (5% to 40% acetonitrile and water, using 0.1% TFA as modifier). Yield 160 mg, 34% for two steps. Ion found by LCMS: [M+H]⁺=527.2.

Step f.

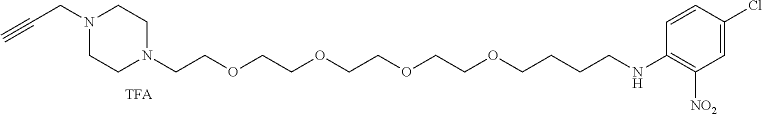 [SMILES: C#CCN1CCN(CCOCCOCCOCCOCCCCNc2ccc(Cl)cc2[N+](=O)[O-])CC1]

A solution of the product from step e (160 mg, 0.212 mmol) in acetic acid (5 ml) was heated at 70° C., and zinc (70 mg, 1.06 mmol) was added. The solution was stirred at 70° C. for 10 mins to give at which time the reaction was complete by LCMS. The crude mixture was filtered, and used in the next step without further purification. LC/MS [M+H]⁺=497.3.

Step g.

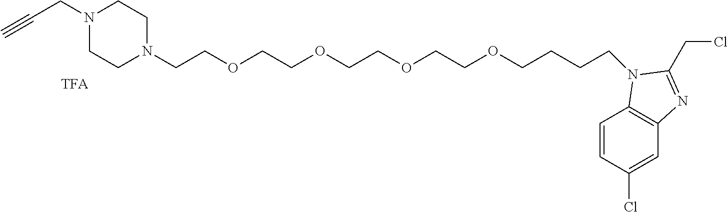 [SMILES: C#CCN1CCN(CCOCCOCCOCCOCCCCn2c(CCl)nc3cc(Cl)ccc32)CC1]

To a solution of the product from step fin acetic acid (5 ml) was added 2-chloro-1,1,1-trimethoxyethane (197 mg, 1.28 mmol). The reaction was stirred at 70° C. for 1 hour. The solution was cooled, concentrated and purified by RPLC (5% to 50% acetonitrile and water, using 0.1% TFA as modifier). Yield 38 mg, 32%. Ion found by LCMS: [M+H]⁺=555.2.

Step h.

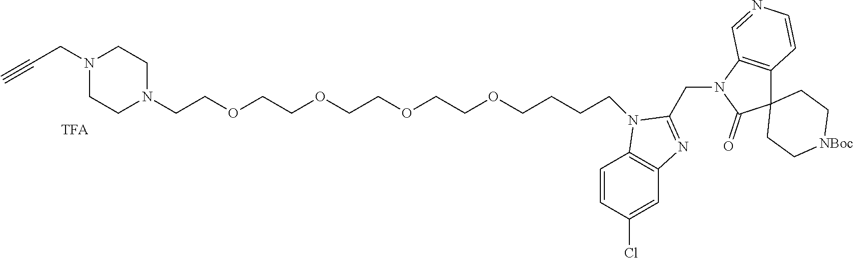 [SMILES: C#CCN1CCN(CCOCCOCCOCCOCCCCn2c(CN3C(=O)C4(CCN(C(=O)OC(C)(C)C)CC4)c4ccncc43)nc3cc(Cl)ccc32)CC1]

To a solution of the product from Example 37 (28 mg, 0.094 mmol) in dry acetonitrile was added Cs₂CO₃ (65.2 mg, 0.2 mmol), followed by the step-g product (38 mg, 0.047 mmol). The reaction solution was stirred at 80° C. for 20 min, cooled, concentrated and purified by RPLC (5% to 50% acetonitrile and water, using 0.1% TFA as modifier). Yield 25 mg, 64%. Ion found by LCMS: [M+H]⁺=822.2.

### Example 43. Synthesis of Int-29

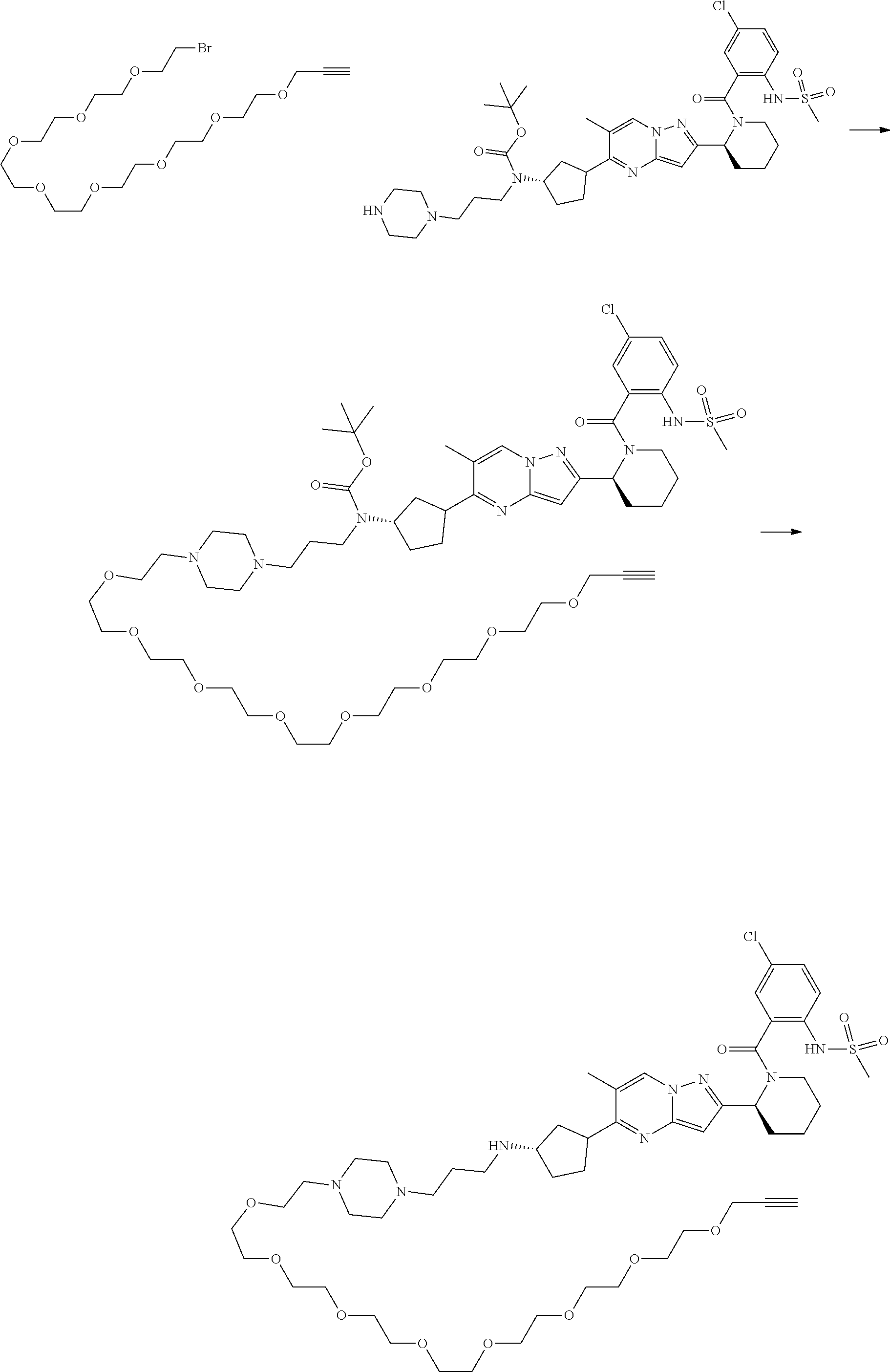 [SMILES: C#CCOCCOCCOCCOCCOCCOCCOCCOCCBr ; Cc1cn2nc([C@@H]3CCCCN3C(=O)c3cc(Cl)ccc3NS(C)(=O)=O)cc2nc1C1CC[C@H](N(CCCN2CCNCC2)C(=O)OC(C)(C)C)C1 ; C#CCOCCOCCOCCOCCOCCOCCOCCOCCN1CCN(CCCN(C(=O)OC(C)(C)C)[C@H]2CCC(c3nc4cc([C@@H]5CCCCN5C(=O)c5cc(Cl)ccc5NS(C)(=O)=O)nn4cc3C)C2)CC1 ; C#CCOCCOCCOCCOCCOCCOCCOCCOCCN1CCN(CCCN[C@H]2CCC(c3nc4cc([C@@H]5CCCCN5C(=O)c5cc(Cl)ccc5NS(C)(=O)=O)nn4cc3C)C2)CC1]

Step a.

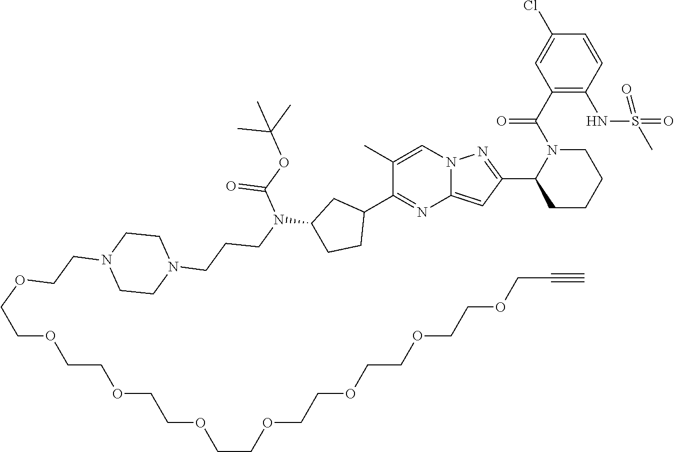 [SMILES: C#CCOCCOCCOCCOCCOCCOCCOCCOCCN1CCN(CCCN(C(=O)OC(C)(C)C)[C@H]2CCC(c3nc4cc([C@@H]5CCCCN5C(=O)c5cc(Cl)ccc5NS(C)(=O)=O)nn4cc3C)C2)CC1]

To a solution of amine TFA salt from Example 19, Int-10 (247.4 mg, 0.284 mmol) and propagyl-PEG8-bromide (206.7 mg, 0.425 mmol) in anhydrous DMF (2.8 mL) at room temperature was added DIEA (0.20 mL, 1.13 mmol). The resulting mixture was heated at 90° C. for 16 hours. It was then cooled down to room temperature, and purified by RPLC (ISCO, 10% to 80% acetonitrile and water, using 0.1% TFA as modifier). Yield 208.2 mg, 48.8%. Ion found by LCMS: [M+H]⁺=574.8.

Step b.

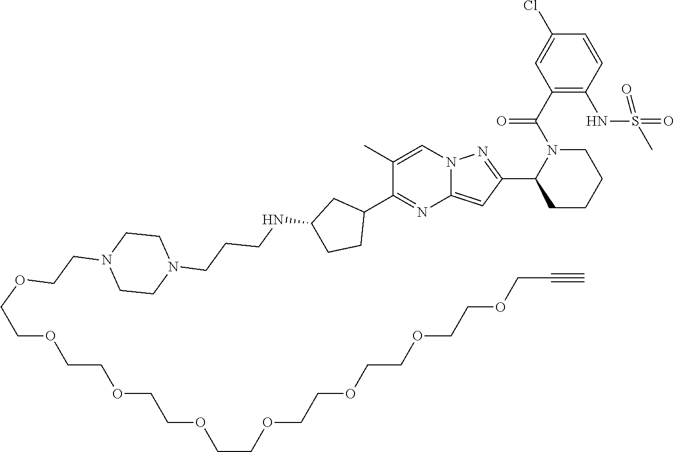 [SMILES: C#CCOCCOCCOCCOCCOCCOCCOCCOCCN1CCN(CCCN[C@H]2CCC(c3nc4cc([C@@H]5CCCCN5C(=O)c5cc(Cl)ccc5NS(C)(=O)=O)nn4cc3C)C2)CC1]

The product from the previous step (208.2 mg, 0.176 mmol) was stirred in TFA (3 mL) and dichloromethane (3 mL) at room temperature. After the reaction was completed, it was concentrated under reduced pressure. The residue was purified by RPLC (ACCQ, 5% to 50% acetonitrile and water, using 0.1% TFA as modifier). Yield 51.0 mg, 22.7% as TFA salt. Ion found by LCMS: [(M+2H)/2]⁺=524.8.

### Example 44. Synthesis of Int-43

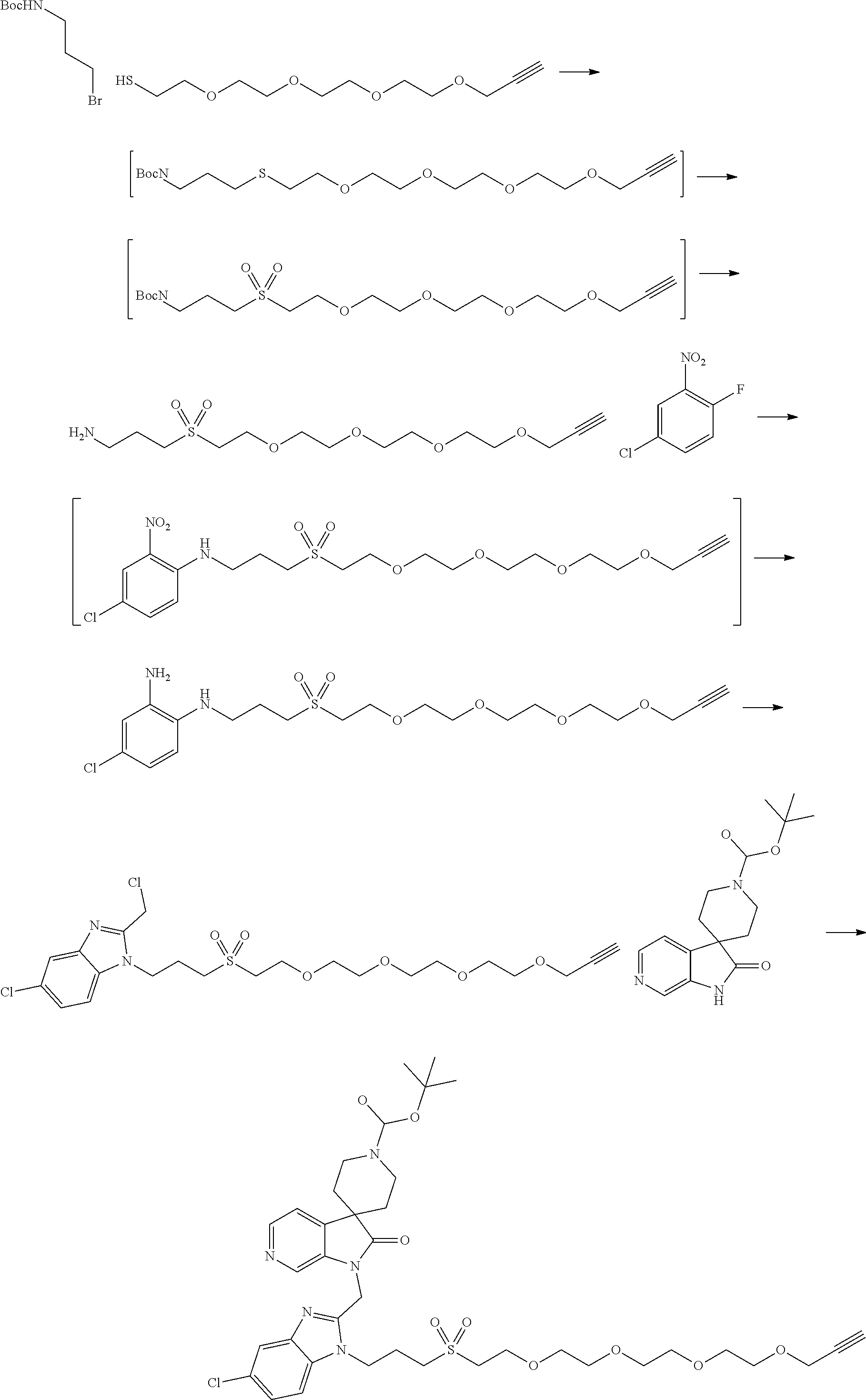

Step a.

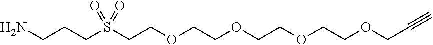 [SMILES: C#CCOCCOCCOCCOCCS(=O)(=O)CCCN]

Propargyl-peg4-thiol (1.0 g, 4.0 mmol) was added to N-boc-amino-propyl bromide (1.1 g, 4.4 mmol) and cesium carbonate (1.1 g, 4.4 mmol) were stirred in refluxing acetonitrile (10 mL) for 4 hours. The mixture was filtered and concentrated to afford the intermediate (LCMS [M+Na]⁺=428.2). The crude intermediate was dissolved in methanol (25 mL) and oxone (7.4 g, 12.1 mmol) was added and the reaction was stirred at 50° C. for 3 hours. The mixture was filtered and concentrated on the rotary evaporator. The sulfone intermediate was purified by RP HPLC (ISCO COMBI FLASH, 5-95% acetonitrile in DI water, 0.1% TFA, 40 minute gradient). The pure fractions were pooled and lyophilized to afford the intermediate as a clear oil (LCMS [M+Na]⁺=460.2). The Boc protected intermediate was stirred in 4N HCl in dioxane (25 mL) at ambient temperature for 30 minutes. The solvent was removed on the rotary evaporator, azeotroped with toluene (3×, 10 mL), and dried under high vacuum to afford the product as a yellow oil. Yield 69%, 3 steps. LC/MS [M+H]⁺=338.2.

Step b.

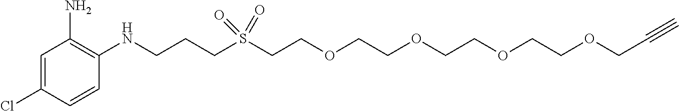 [SMILES: C#CCOCCOCCOCCOCCS(=O)(=O)CCCNc1ccc(Cl)cc1N]

The intermediate from step a. of this example (625 mg, 1.85 mmol) was added to a mixture of 2-fluoro-5-chloro-nitrobenzene (295 mg, 1.68 mmol) and DIPEA (434 mg, 3.3 mmol) in ethanol (10 mL) and the reaction was stirred at 90° C. for 12 hours at which point the starting material had been fully converted to product. The mixture was concentrated and purified by silica gel chromatography (Isco Combi Flash, 0-100% ethyl acetate in hexanes, 25 minute gradient). The pure fractions were pooled and lyophilized to afford the product as a clear oil, (LC/MS[M+H]⁺=493.2). The nitro intermediate was taken up in glacial acetic acid (20 ml) and zinc (150 mg, 2.30 mmol) was added and the mixture was stirred for 1 hour at 50° C. The reaction mixture was filtered and concentrated on the rotary evaporator. The product was taken forward without further purification. Yield 63%, 2 steps. LC/MS [M+H]⁺=463.2.

Step c.

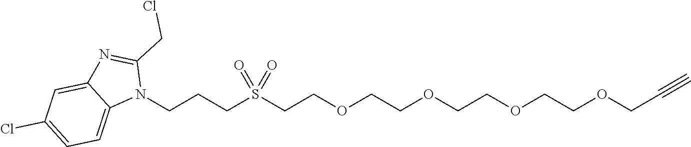 [SMILES: C#CCOCCOCCOCCOCCS(=O)(=O)CCCn1c(CCl)nc2cc(Cl)ccc21]

2-Chloromethyl-1,1,1-trimethoxy ethane (1.08 g, 7.02 mmol) was added to the diamine intermediate, described in step-b of this example, (650 mg, 1.40 mmol) dissolved in glacial acetic acid (15 mL) and the reaction was stirred at 70° C. for 2 hours. The mixture was concentrated on the rotary evaporator and purified by silica gel chromatography (Isco Combi Flash, 0 to 4% methanol/DCM, 25 minute gradient). The pure fractions were pooled and concentrated to afford the product as a brown oil. Yield 71%. LC/MS [M+H]⁺=521.2.

Step d.

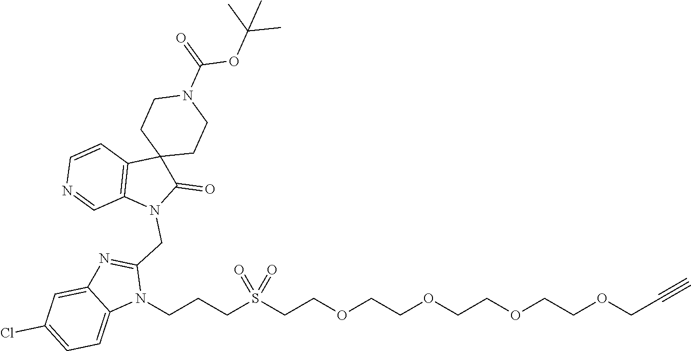 [SMILES: C#CCOCCOCCOCCOCCS(=O)(=O)CCCn1c(CN2C(=O)C3(CCN(C(=O)OC(C)(C)C)CC3)c3ccncc32)nc2cc(Cl)ccc21]

The product of Example 37 (149 mg, 0.49 mmol), the intermediate from step-c of this example (307 mg, 0.59 mmol), and cesium carbonate (192 mg, 0.59 mmol) were stirred together in DMF (2 mL) at ambient temperature for 12 hours. The reaction mixture was applied directly to by RP HPLC (ACCQ semi-prep, 5 to 95% acetonitrile/water, no modifier, 35 minute gradient). The pure fractions were pooled and lyophilized to afford the product as a yellow, hygroscopic solid. Yield 51%. LC/MS [M+H]⁺=788.4.

### Example 45. Synthesis of Int-36

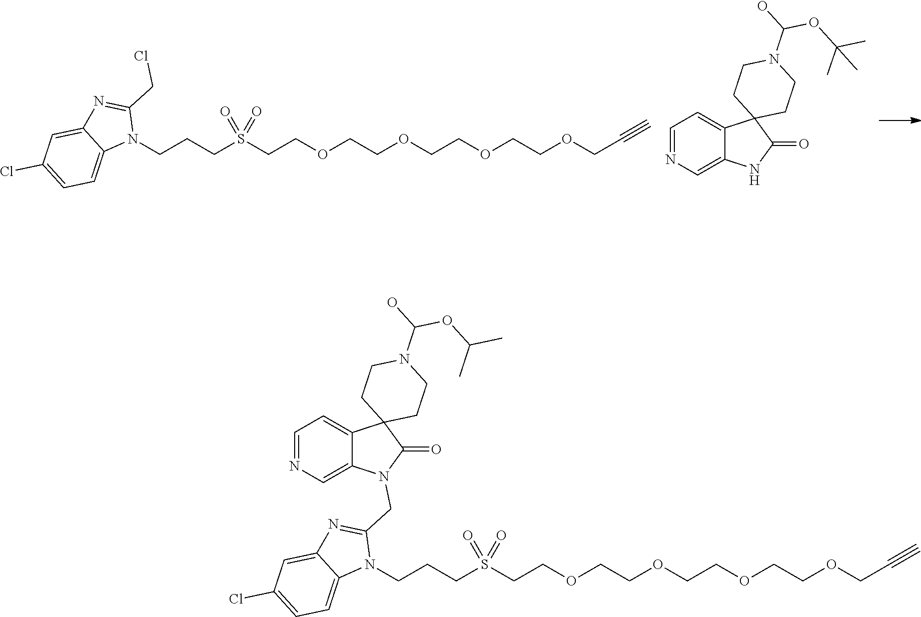

The product of Example 38 (66 mg, 0.23 mmol), the intermediate from step c. of Example 44 (Synthesis of Int-43) (120 mg, 0.23 mmol), and cesium carbonate (75 mg, 0.23 mmol) were stirred together in DMF (2 mL) at ambient temperature for 12 hours. The reaction mixture was applied directly to by RP HPLC (ACCQ semi-prep, 5-95% acetonitrile in DI water, no modifier, 35 minute gradient). The pure fractions were pooled and lyophilized to afford the product as a yellow, hygroscopic solid. Yield 39%. LC/MS [M+H]⁺=773.8.

### Example 46. Synthesis of Int-41

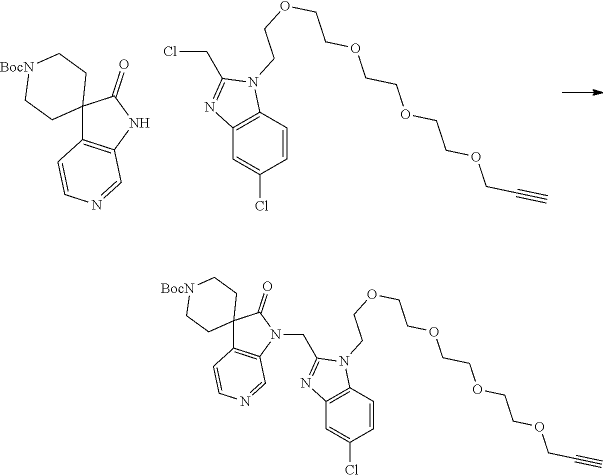 [SMILES: CC(C)(C)OC(=O)N1CCC2(CC1)C(=O)Nc1cnccc12 ; C#CCOCCOCCOCCOCCn1c(CCl)nc2cc(Cl)ccc21 ; C#CCOCCOCCOCCOCCn1c(CN2C(=O)C3(CCN(C(=O)OC(C)(C)C)CC3)c3ccncc32)nc2cc(Cl)ccc21]

To a solution of tert-butyl 2-oxospiro[2-pyrrolino[2,3-c]pyridine-3,4′-piperidine]-10-carboxylate (31 mg, 0.10 mmol prepared as described in Example 37) in CH₃CN (10 mL) was added Cs₂CO₃ (100 mg, 0.30 mmol). The solution was stirred for 20 min. To this was added 1-{2-[5-chloro-2-(chloromethyl)benzimidazolyl]ethoxy}-2-[2-(2-prop-2-ynyloxyethoxy)ethoxy]ethane (42 mg, 0.10 mmol prepared as described in Example 39) and the solution was stirred for 16 h. The excess CH₃CN was removed and the crude material was purified by reversed phase HPLC (0-100% CH₃CN/H₂O using 0.1% TFA). Yield 34 mg, 50.0%. Ion found by LCMS: [M+H]⁺=682.2.

### Example 47. Synthesis of Int-42

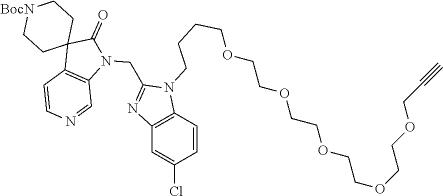 [SMILES: C#CCOCCOCCOCCOCCOCCCCn1c(CN2C(=O)C3(CCN(C(=O)OC(C)(C)C)CC3)c3ccncc32)nc2cc(Cl)ccc21]

The title compound was prepared analogously to Example 46 (Int-41) from tert-butyl 2-oxospiro[2-pyrrolino[2,3-c]pyridine-3,4′-piperidine]-10-carboxylate and 1-{4-[5-chloro-2-(chloromethyl)benzimidazolyl]butoxy}-2-{2-[2-(2-prop-2-ynyloxyethoxy)ethoxy]ethoxy}ethane (prepared as described in Example 36). Ion found by LCMS: [M+H]⁺=754.2.

### Example 48. Synthesis of Int-44

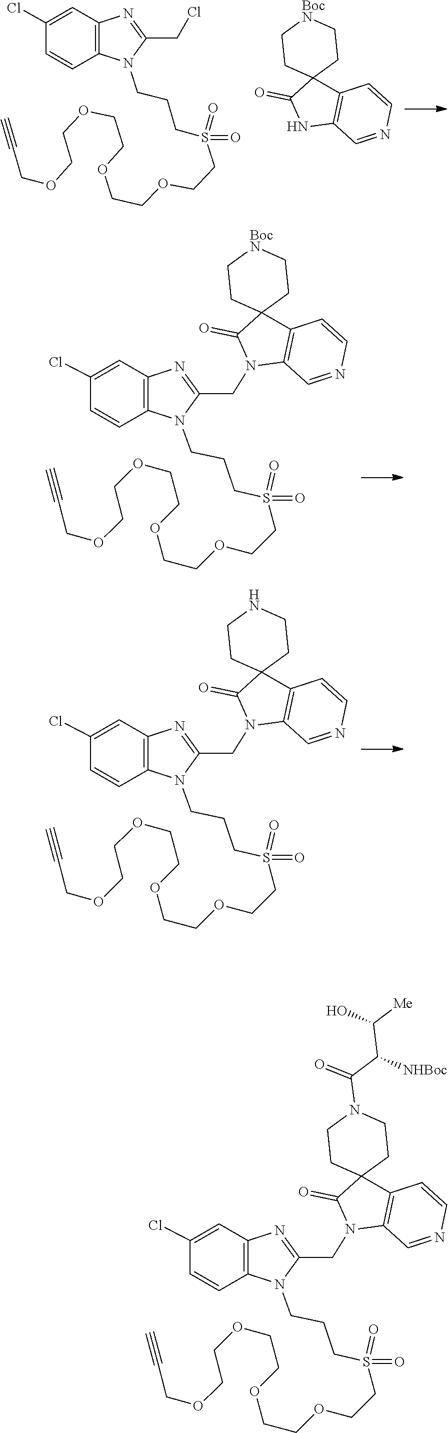 [SMILES: C#CCOCCOCCOCCOCCS(=O)(=O)CCCn1c(CCl)nc2cc(Cl)ccc21 ; CC(C)(C)OC(=O)N1CCC2(CC1)C(=O)Nc1cnccc12 ; C#CCOCCOCCOCCOCCS(=O)(=O)CCCn1c(CN2C(=O)C3(CCN(C(=O)OC(C)(C)C)CC3)c3ccncc32)nc2cc(Cl)ccc21 ; C#CCOCCOCCOCCOCCS(=O)(=O)CCCn1c(CN2C(=O)C3(CCNCC3)c3ccncc32)nc2cc(Cl)ccc21 ; C#CCOCCOCCOCCOCCS(=O)(=O)CCCn1c(CN2C(=O)C3(CCN(C(=O)[C@@H](NC(=O)OC(C)(C)C)[C@@H](C)O)CC3)c3ccncc32)nc2cc(Cl)ccc21]

Step a.

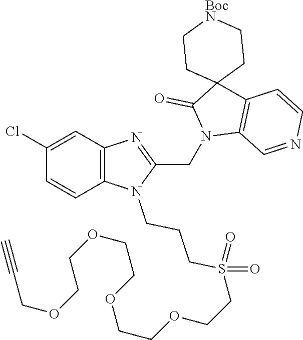 [SMILES: C#CCOCCOCCOCCOCCS(=O)(=O)CCCn1c(CN2C(=O)C3(CCN(C(=O)OC(C)(C)C)CC3)c3ccncc32)nc2cc(Cl)ccc21]

To a solution of tert-butyl 2-oxospiro[2-pyrrolino[2,3-c]pyridine-3,4′-piperidine]-10-carboxylate (52 mg, 0.17 mmol, prepared as described in Example 37) in CH₃CN (10 mL) was added Cs₂CO₃ (140 mg, 0.43 mmol). The solution was stirred for 20 min. To this was added 1-({3-[5-chloro-2-(chloromethyl)benzimidazolyl]propyl}sulfonyl)-2-{2-[2-(2-prop-2-ynyloxyethoxy)ethoxy]ethoxy}ethane (90 mg, 0.17 mmol prepared as described in Example 44, Int-43) and the solution was stirred for 16 h. The excess CH₃CN was removed and the crude mixture was adsorbed onto CELITE® and purified via flash chromatography (0-50% MeOH and Ethyl acetate). Yield 56 mg, 41%. Ion found by LCMS: [M+H]⁺=788.6.

Step b.

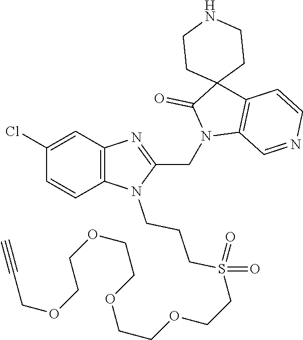 [SMILES: C#CCOCCOCCOCCOCCS(=O)(=O)CCCn1c(CN2C(=O)C3(CCNCC3)c3ccncc32)nc2cc(Cl)ccc21]

4N HCl in Dioxane (6 mL) was added to tert-butyl 1-[(5-chloro-1-{3-[(2-{2-[2-(2-prop-2-ynyloxyethoxy)ethoxy]ethoxy}ethyl)sulfonyl]propyl}benzimidazol-2-yl)methyl]-2-oxospiro[2-pyrrolino[2,3-c]pyridine-3,4′-piperidine]-10-carboxylate (28 mg, 0.04 mmol) and the solution was stirred for 3 h. The excess solvent was removed and the crude material was used in the next step without further purification. LCMS: [M+H]+=688.2.

Step c.

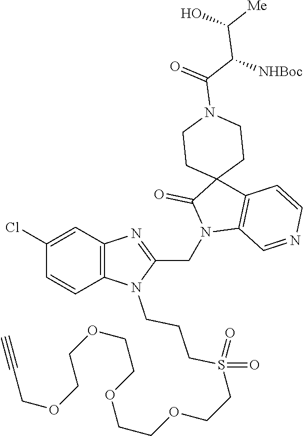 [SMILES: C#CCOCCOCCOCCOCCS(=O)(=O)CCCn1c(CN2C(=O)C3(CCN(C(=O)[C@@H](NC(=O)OC(C)(C)C)[C@@H](C)O)CC3)c3ccncc32)nc2cc(Cl)ccc21]

To crude 1-[(5-chloro-1-{3-[(2-{2-[2-(2-prop-2-ynyloxyethoxy)ethoxy]ethoxy}ethyl) sulfonyl]propyl}benzimidazol-2-yl)methyl]spiro[2-pyrrolino[2,3-c]pyridine-3,4′-piperidine]-2-one (25 mg, 0.040 mmol assuming 100% yield) in DMF (5 mL) was added Boc-Thr-OH (10 mg, 0.05 mmol) followed by Hünigs base (2 drops). EDCI (15 mg, 0.08 mmol) and HOBt (15 mg, 0.08 mmol) were then added and the solution was stirred for 16 h. The excess solvent was removed and the crude material was purified by reversed phase HPLC (0-100% CH₃CN/H₂O using 0.1% TFA). Yield 28 mg, 79%. Ion found by LCMS: [M+H]+=889.1.

### Example 49. Synthesis of Int-45

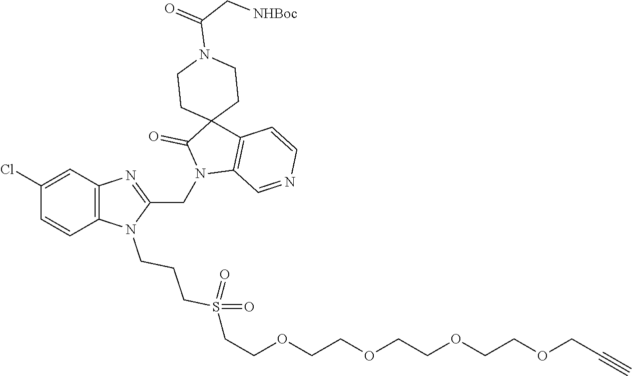 [SMILES: C#CCOCCOCCOCCOCCS(=O)(=O)CCCn1c(CN2C(=O)C3(CCN(C(=O)CNC(=O)OC(C)(C)C)CC3)c3ccncc32)nc2cc(Cl)ccc21]

The title compound was prepared analogously to Example 48 (Int-44) from crude 1-[(5-chloro-1-{3-[(2-{2-[2-(2-prop-2-ynyloxyethoxy)ethoxy]ethoxy}ethypsulfonyl]propyl}benzimidazol-2-yl)methyl]spiro[2-pyrrolino[2,3-c]pyridine-3,4′-piperidine]-2-one and Boc-Gly-OH. Ion found by LCMS: [M+H]⁺=845.2.

### Example 50. Synthesis of Int-37

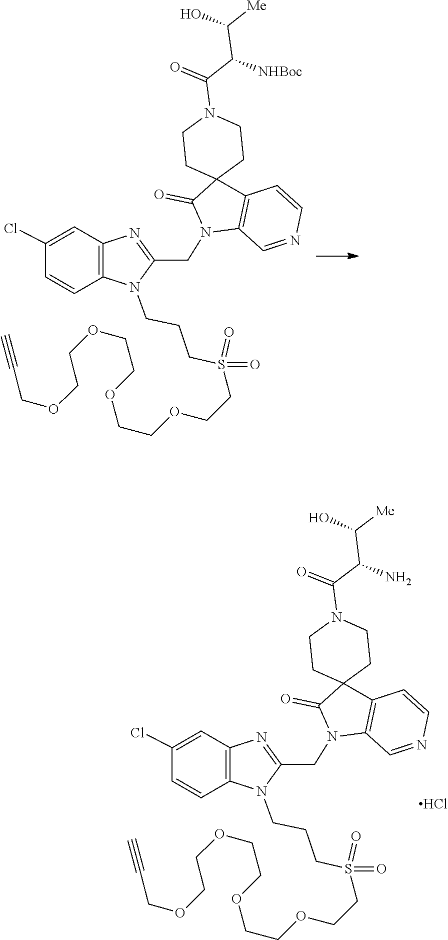 [SMILES: C#CCOCCOCCOCCOCCS(=O)(=O)CCCn1c(CN2C(=O)C3(CCN(C(=O)[C@@H](NC(=O)OC(C)(C)C)[C@@H](C)O)CC3)c3ccncc32)nc2cc(Cl)ccc21 ; C#CCOCCOCCOCCOCCS(=O)(=O)CCCn1c(CN2C(=O)C3(CCN(C(=O)[C@@H](N)[C@@H](C)O)CC3)c3ccncc32)nc2cc(Cl)ccc21 ; Cl]

4N HCl in Dioxane (6 mL) was added to N-(1-((1R)-1-hydroxyethyl)(1S)-2-{1-[(5-chloro-1-{3-[(2-{2-[2-(2-prop-2-ynyloxyethoxy)ethoxy]ethoxy}ethyl)sulfonyl]propyl}benzimidazol-2-yl)methyl]-2-oxospiro[2-pyrrolino[2,3-c]pyridine-3,4′-piperidine]-10-yl}-2-oxoethyl)(tert-butoxy)carboxamide (15 mg, 0.020 mmol, described in Example 48, Int-43) and the solution was stirred for 1 h. The excess solvent was removed to afford pure product. Yield 14 mg, quant. Ion found by LCMS: [M+H]+=789.2.

### Example 51. Synthesis of Int-38

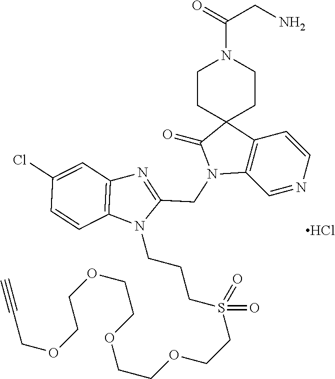 [SMILES: C#CCOCCOCCOCCOCCS(=O)(=O)CCCn1c(CN2C(=O)C3(CCN(C(=O)CN)CC3)c3ccncc32)nc2cc(Cl)ccc21 ; Cl]

The title compound was prepared analogously to Example 52 (Int-37) from crude (tert-butoxy)-N-(2-{1-[(5-chloro-1-{3-[(2-{2-[2-(2-prop-2-ynyloxyethoxy)ethoxy]ethoxy}ethyl) sulfonyl]propyl}benzimidazol-2-yl)methyl]-2-oxospiro[2-pyrrolino[2,3-c]pyridine-3,4′-piperidine]-10-yl}-2-oxoethyl)carboxamide (described in Example 49, Int-45). Ion found by LCMS: [M+H]+=745.2.

### Example 52. Synthesis of Conjugate 19

The title conjugate was prepared according to the general conjugation procedure using alkyne derivatized small molecule described in Example 47, Int-42 with azido functionalized Fc (SEQ ID NO: 63) DAR 6.0. Yield 21.0 mg, 18.0%. MALDI−TOF=61283.

### Example 53. Microneutralization Assay for Quantifying RSV Neutralizing Compounds

HEp-2 cells (ATCC #CCL-23) were seeded at 5E4/well/200 μL in DMEM (Fisher cat #11965118) supplemented with 10% heat-inactivated (HI) fetal bovine serum (HI-FBS; Fisher cat #10-082-147), 1× penicillin-streptomycin (P/S, 100 μg/mL; Fisher cat #MT30002C), and 1× L-glutamine (L-Gln, 2 mM; Fisher cat #25030-164) in 96-well tissue culture treated plates (Fisher cat #08-772-17) and incubated overnight at 37° C. and 5% CO₂. Duplicate 10-fold serial dilutions of compound in DMEM with 2% HI-FBS, 1× P/S, 2 mM L-Gln were prepared in 96-well tissue culture plates at 60 μL/well. The RSV Long strain (RetroVirox, San Diego), RSV A2 strain (Virapur, San Diego), RSV B1 strain (RetroVirox, San Diego), or RSV strain CH18537 (RetroVirox, San Diego), were diluted in DMEM with 2% HI-FBS, 1× P/S, 2 mM L-Gln and 60 μL/well added to 60 μL compound for a multiplicity of infection (MOI) of 0.002 (Long, A2), 0.003 (B1), or 0.004 (CH18537). Virus only (no drug) and cells only (no virus, no drug) controls were included on each plate. The compound-virus mix was incubated for 1 h at 37° C. and 5% CO₂. After 1 h, medium was removed from HEp-2 cells by aspiration and wells washed once with 100 μL/well 1×PBS pH 7.4 (Fisher cat #MT21040CM). The compound-virus mix (100 μL/well) was added to the cells and incubated for 1 h at 37° C. and 5% CO₂ prior to addition of 100 μL/well of DMEM with 2% HI-FBS, 1× P/S, 2 mM L-Gln (200 μL final vol) and incubation at 37° C. and 5% CO₂. On day 6 post infection, supernatant was aspirated and cells fixed with 100 μL/well ice-cold 80% acetone in 1×PBS for 20 min at 4° C. The acetone was aspirated and plates air-dried for 20 min at room temperature. The plates were washed 3× with 200 μL/well 1×PBS, 0.05% Tween-20 (PBST) and blocked with 200 μL/well 5% non-fat dry milk (Fisher cat #50-195-952) in PBST for 1 h with shaking (500 rpm) on an orbital plate shaker. The blocking solution was discarded and cells incubated with 100 μL/well mouse anti-F protein antibody (MilliporeSigma cat #MAB8599) diluted 1:2,000 in diluent (blocking solution diluted 1:1 in 1×PBS pH 7.4) for 2 h with shaking. Plates were washed 5× with 300 μL/well PBST and incubated with 100 μL/well HRP-conjugated goat anti-mouse IgG antibody (SouthernBiotech cat #1030-05) diluted 1:1,000 in diluent for 1 h with shaking. Plates were washed 5× with 300 μL/well PBST and incubated with 100 μL/well TMB substrate (Fisher cat #BDB555214) for ˜5 min. The reaction was stopped with 100 μL/well 1N H₂SO₄. Absorbance was read at 450 nm with an EnSpire multimode plate reader (PerkinElmer). The mean A450 for the cells only control was subtracted from all A450 values, and the percent virus neutralisation calculated for each compound concentration, relative to the virus only control. Half maximal effective concentration (EC₅₀) was calculated with GraphPad Prism version 8 using nonlinear regression analysis of % neutralisation vs. log₁₀ concentration plots.

The 50% cytotoxic concentration (CC₅₀) at day 6 post infection was determined for assays run in parallel with the microneutralization assay. Briefly, the medium was removed by aspiration and the cells fixed/stained for 1 h at room temperature with 60 μL/well crystal violet solution (0.1% crystal violet, 20% methanol, 3% paraformaldehyde). The stain was removed and wells washed 3× times with 100 μL/well 1×PBS pH 7.4. Plates were air-dried for 2 h at room temperature and the absorbance at 570 nm read with an EnSpire multimode plate reader (PerkinElmer). Compound concentration (μM) required for the reduction of cell viability by 50% (CC₅₀) was calculated with GraphPad Prism version 8 using nonlinear regression analysis.

In the microneutralization assay, Int-33 (a JNJ179 derivative) was more potent than benchmark compounds Presatovir (GS-5806), and VP-14637 but not parent compound JNJ179 (Table 6). Conjugate 19 is the most potent conjugate to date (EC₅₀ 1.5-2.3 nM).

TABLE 6
RSV microneutralization EC₅₀ at MOI = 0.002
(Long, A2), 0.003 (B1), or 0.004 (CH18537).

| | RSV Long | RSV A2 | RSV B1 | RSV CH18537 | Cytotoxicity |
|---|---|---|---|---|---|
| Compound | EC₅₀ (nM) | EC₅₀ (nM) | EC₅₀ (nM) | EC₅₀ (nM) | CC₅₀ (nM) |
| Presatovir/GS-5806 | 8.4E−03 | 1.4E−02 | 8.2E−01 | 3.2E−01 | nr |
| benchmark | | | | | |
| JNJ179 benchmark | <1.0E−06 | nr | nr | nr | nr |
| VP14637 benchmark | 9.0E−02 | nr | nr | nr | nr |
| Int-01 | 9.8E+01 | nr | nr | nr | nr |
| Int-02 | 1.1E+02 | nr | nr | nr | nr |
| Int-03 | >1.0E+03 | nr | nr | nr | nr |
| Int-04 | >1.0E+03 | nr | nr | nr | nr |
| Int-05 | >1.0E+03 | nr | nr | nr | nr |
| Int-06 | 1.5E+02 | nr | nr | nr | nr |
| Int-07 | 1.8E+01 | nr | nr | nr | nr |
| Int-08 | 4.3E+01 | nr | nr | nr | nr |
| Int-09 | 1.5E+03 | nr | nr | nr | nr |
| Int-10 | 9.7E+01 | nr | nr | nr | nr |
| Int-23 | >1.0E+03 | nr | nr | nr | nr |
| Int-24 | >1.0E+03 | nr | nr | nr | nr |
| Int-25 | 9.2E+01 | nr | nr | nr | nr |
| Int-26 | 1.7E+02 | nr | nr | nr | nr |
| Int-27 | 2.0E+02 | nr | nr | nr | nr |
| Int-28 | 1.5E+02 | nr | nr | nr | nr |
| Int-29 | 1.3E+02 | 1.7E+02 | nr | nr | nr |
| Int-30 | >1.0E+03 | nr | nr | nr | nr |
| Int-31 | 1.3E+02 | nr | nr | nr | nr |
| Int-32 | >1.0E+03 | nr | nr | nr | nr |
| Int-34 | 1.3E−01 | nr | nr | nr | nr |
| Int-35 | 3.4E−01 | 7.4E−01 | nr | nr | nr |
| Int-41 | 1.4E+01 | 1.5E+01 | 3.6E+00 | nr | nr |
| Int-42 | 2.2E−01 | 8.8E−01 | nr | nr | nr |
| Int-43 | <1.0E−06 | <1.0E−06 | nr | nr | nr |
| Synagis (palivizumab) | 1.7E+01 | 2.1E+01 | nr | nr | nr |
| Conjugate 1 | 4.5E+02 | nr | nr | nr | >2.0E+04 |
| Conjugate 2 | 4.6E+01 | nr | nr | nr | nr |
| Conjugate 3a | 2.7E+02 | nr | nr | nr | >2.0E+04 |
| Conjugate 3b | 1.3E+02 | nr | nr | nr | >2.0E+04 |
| Conjugate 4 | 5.9E+02 | nr | nr | nr | >2.0E+04 |
| Conjugate 5 | 1.8E+02 | nr | nr | nr | >2.0E+04 |
| Conjugate 6 | 3.5E+01 | nr | nr | nr | >2.0E+04 |
| Conjugate 7 | 2.3E+02 | nr | nr | nr | nr |
| Conjugate 8 | 1.3E+02 | nr | nr | nr | >2.0E+04 |
| Conjugate 9 | 9.8E+01 | nr | nr | nr | >2.0E+04 |
| Conjugate 11 | 1.5E+02 | nr | nr | nr | >2.0E+04 |
| Conjugate 12 | 8.7E+01 | nr | nr | nr | >2.0E+04 |
| Conjugate 13 | 6.3E+01 | nr | nr | nr | >2.0E+04 |
| Conjugate 14 | 7.5E+01 | nr | nr | nr | >2.0E+04 |
| Conjugate 15a | nr | nr | nr | nr | nr |
| Conjugate 15b | nr | nr | nr | nr | nr |
| Conjugate 16 | nr | nr | nr | nr | nr |
| Conjugate 17 | 1.4E+02 | nr | nr | nr | >2.0E+04 |
| Conjugate 18 | 3.1E+01 | 4.0E+01 | nr | nr | >2.0E+04 |
| Conjugate 19 | 1.5E+00 | 2.3E+00 | nr | nr | >2.0E+04 |

nr, not run.

### Example 54. RSV Plaque Reduction Assay

An RSV plaque reduction assay was performed according to the following protocol. On Day 1, HEP-2 cells were seeded in 24-well plates at 5×10E5 cells/well/500 ul. On Day 2, once the cells are at 100% confluency, the cells were infected at a viral dilution of approximately 30 plaques per well in DMEM+2% FBS infection buffer and treated with compound. The medium was aspirated and cells were infected with 90 ul of viral dilution per well. Then, cells were treated with 10 ul of 10× compound resulting in a final concentration of between 1 nM-10 uM of the compound. The cells were incubated for 2 h at room temperature, rocking every 15 minutes. After 2 h, the inoculum/compound mixture is removed. 450 ul of overlay media (1 part Avicel 2.5%; 1 part DMEM+2% FBS) was added to the well and the cells were treated with 50 ul of 10× compound resulting in a final concentration of 1 nM-10 uM of the compound. The cells were incubated for 6 days at 37 deg C.

For staining and quantification: The infected cell plates were fixed with 100% methanol for 10 to 30 minutes. The plates were washed three times with 5% milk/PBS, 1 mL/well. A primary antibody, goat anti-RSV polyclonal antibody (Chemicon Cat #AB1128), diluted in 5% milk/PBS at 1:1000 was put onto each well. The plate was placed in a shaker and incubated at room temperature for 1 hour, after which the plates were washed three times with 5% milk/PBS. A peroxidase conjugated secondary antibody, ImmunoPure rabbit anti-goat antibody IgG (H+L) (ThermoScientific, Cat #31402) at 1:1000, was diluted in 5% milk/PBS and added to the plate, after which the plate was placed on a shaker at room temperature for 1 hour. Plates were then washed with 1×PBS, three times, after which True Blue substrate (KPL), 0.3 ml/well for 12-well plates, was added for 10 minutes at room temperature. The plates were rinsed 3 times with deionized water, air-dried and the number of plaques was counted.

The results of the plaque reduction assay are presented in Table 7.

TABLE 7
RSV Plaque reduction assay

| | | EC₅₀ (nM) | |
|---|---|---|---|
| Compound | Targeting | RSV Long | RSV B1 |
| tested | moiety | MOI 0.0001 | MOI 0.0001 |
| Conjugate 19 | Int-42 | 15 | 7.7 |
| Int-42 | n/a | 17 | 13 |

### Example 55. Synthesis of a Conjugate Including an Fc Domain Having a C220S/YTE Quadruple Mutation

Preparation of the Click reagent solution: 0.0050M CuSO₄ in PBS buffer solution: 10.0 mg CuSO₄ was dissolved in 12.53 mL PBS, then took 5.00 mL this CuSO₄ solution and added 43.1 mg BTTAA (CAS #1334179-85-9) and 247.5 mg sodium ascorbate to give the Click reagent solution (0.0050M CuSO4, 0.020M BTTAA and 0.25M sodium ascorbate).

To a solution of azido functionalized Fc having a C220S mutation and a YTE mutation (65.5 mg, 10.0 mL, 1.13 μmol, azido DAR˜5.9, SEQ ID NO: 67) in a 15 mL centrifuge tube was added to an alkyne derivatized small molecule (3.0 equivalents per each azido of the Fc). After gently agitating to dissolve all solids, the mixture was treated with the Click reagent solution (1.80 mL). The resulting mixture was gently rotated for 12 hours at ambient temperature. It was purified by affinity chromatography over a protein A column, followed size exclusion chromatography (see general conjugate purification protocol). Maldi TOF analysis of the purified final product gave an average mass of 66,420 Da (DAR=5.8). Yield 57 mg with 98% purity.

### Example 56. 30-Day Comparative Non-Human Primate PK Study Following IV Administration of a Conjugate Including an Fc Domain Having a C220S/YTE Quadruple Mutation

A conjugate including an Fc domain having a C220S mutation and a YTE mutation (SEQ ID NO: 67) was synthesized as described in Example 55. A non-human primate PK study was performed to compare IV administration of the C220S/YTE Fc conjugate (SEQ ID NO: 67) to a conjugate including an Fc domain having a C220S mutation alone (SEQ ID NO: 64).

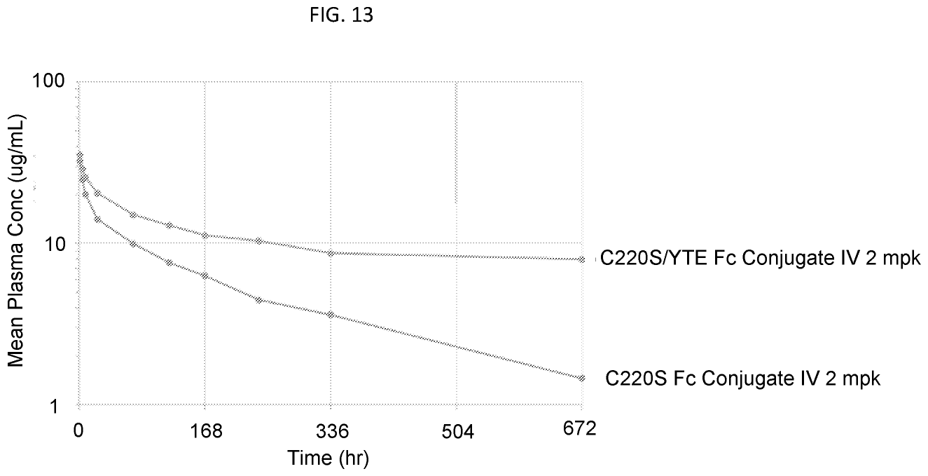

Non-human primate (NHP) PK studies were performed by BTS Research (San Diego, Calif.) using male and female cynomolgus monkeys 5-9 years old with body weights ranging from 3.5-8.5 kg. NHPs were injected IV with 2 mg/kg of test article (0.4 mL/kg dose volume). Animals were housed under standard IACUC approved housing conditions. At appropriate times animals were non-terminally bled (via femoral or cephalic veins) with blood collected in K₂EDTA tubes to prevent coagulation. Collected blood was centrifuged (2,000×g, for 10 minutes) and plasma withdrawn for analysis of test article concentrations over time. The plasma concentrations for the C220S/YTE Fc conjugate and the C220S conjugate at each time point were measured by sandwich ELISA. Briefly, test articles were captured on Fc-coated plates and then detected using a HRP-conjugated anti-human IgG-Fc antibody. Protein concentrations were calculated in GraphPad Prism using 4PL non-linear regression of the C220S/YTE Fc conjugate or C220S conjugate standard curves. A more detailed method description is provided above. The corresponding curves are shown in FIG. 13. The C220S/YTE Fc conjugate demonstrates a significantly improved terminal half-life of ˜45 days compared with ˜10 days for the C220S Fc conjugate. AUCs for the C220S/YTE Fc conjugate are 2× greater than the AUCs for The C220S conjugate (Table 8).

TABLE 8
Monkey PK, C220S/YTE Fc conjugate vs. C220S Fc conjugate

| | | | | Time (hr) | | | | | | | | | | | | | |
|---|---|---|---|---|---|---|---|---|---|---|---|---|---|---|---|---|---|
| | | | | 0.25 | 4 | 8 | 24 | 72 | 120 | 168 | 240 | 336 | 672 | | | | |
| Dose | | | | Conc | | | | | | | | | | Tmax | Cmax | AUClast | Half-life |
| (mg/kg) | Route | Conjugate | | (ug/mL) | | | | | | | | | | (hr) | (ug/mL) | (hr*ug/mL) | (hr) |
| 2 | IV | C220S | Mean | 32.6 | 24.8 | 20.1 | 14.1 | 9.97 | 7.61 | 6.33 | 4.47 | 3.62 | 1.47 | 0.25 | 32.6 | 3450 | 249 |
| 2 | IV | C220S/YTE | Mean | 35.4 | 29 | 25.7 | 20.5 | 15.1 | 13 | 11.2 | 10.4 | 8.71 | 7.97 | 0.25 | 35.4 | 7210 | 1080 |

### Example 57. 14-Day Mouse PK Study Comparing Plasma and Epithelial Lining Fluid (ELF) Concentrations of a Conjugate Including an Fc Domain

Female BALB/c mice from Charles River Laboratories were allowed to acclimate for 5 days prior to study commencement. Animals were housed 3-6 per cage with free access to food and water. All procedures were performed to NeoSome IACUC policies and guidelines. Mice were injected subcutaneously (SC) with 20 mg/kg of a conjugate having an Fc domain (SEQ ID NO: 64) decorated with one or more small molecule antiviral inhibitors (10 mL/kg dose volume). At selected time points, 3 mice were euthanized by CO₂ inhalation. Blood was collected through cardiac puncture into K₂EDTA tubes for plasma retention. Following blood collection, a bronchoalveolar lavage (BAL) was performed by exposing the trachea, inserting a 23G tubing adaptor, and performing 2×0.5 mL flushes with sterile 1×PBS pH 7.4. The recovered fluid volume was recorded and retained. Once the BAL procedure was complete, the lungs were removed, weighed and stored at −80° C. Aliquots of the plasma and BAL fluid (BALF) were decanted prior to −80° C. storage of the samples for use in a urea quantification assay. The collected BALF was centrifuged at 12,000 RPM for 5 minutes at room temperature to pellet the alveolar macrophages with both the pellet and supernatant stored at −80° C. until shipment to sponsor. The plasma concentrations for the conjugate at each time point were measured by indirect ELISA as described above. Briefly, the conjugate molecules were captured on neuraminidase (NA) coated plates and then detected using a HRP-conjugated anti-human IgG Fcγ specific F(ab′)₂. The same ELISA was performed on BALF harvested as described above. The conjugate plasma concentrations were calculated in GraphPad Prism using 4PL non-linear regression of the conjugate standard curves. ELF volume and conjugate concentration in ELF was determined using urea as a dilution marker as described previously (Rennard et al., 1986 J Appl Physiol 60:532-538). The curves comparing conjugate to ELF levels are shown in FIG. 14. By 2 h post injection, conjugate epithelial lining fluid (ELF) levels are ˜60% of plasma exposure levels (AUCs) across the rest of the time course indicating nearly immediate partitioning of conjugate 45 from plasma to the ELF in the lung (FIG. 14, Table 9).

TABLE 9
Conjugate plasma and ELF levels in mice over 2 weeks.

| | Time | | | | | | | | | | | | |
|---|---|---|---|---|---|---|---|---|---|---|---|---|---|
| | (hr) | | | | | | | | | | | | |
| | 1 | 2 | 4 | 8 | 24 | 48 | 72 | 120 | 168 | 336 | | | |
| | Conc | | | | | | | | | | Tmax | Cmax | AUClast |
| Group | (ug/mL) | | | | | | | | | | (hr) | (ug/mL) | (hr*ug/mL) |
| ELF | 5.61 | 29.9 | 70.6 | 98.4 | 149 | 105 | 94.2 | 49.5 | 47.4 | 16.1 | 24 | 149 | 19000 |
| Plasma | 30.7 | 63.9 | 110 | 180 | 197 | 178 | 144 | 104 | 87 | 29.4 | 24 | 197 | 32500 |

1. A conjugate described by any one of formulas (D-I), (M-I), (1), or (2): 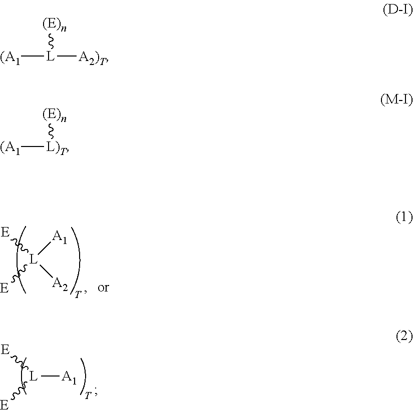
wherein each A₁ and each A₂ is independently selected from any one of formulas (A-I)-(A-III): 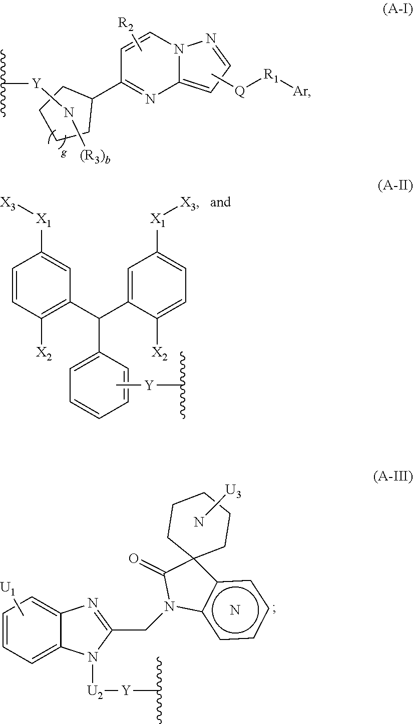
wherein
Q is selected from optionally substituted C₁-C₂₀ alkyl, optionally substituted C₃-C₂₀ cycloalkyl, optionally substituted C₂-C₂₀ heterocycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;
R₁, X₁, and Y are each independently selected from —O—, —S—, —NR₅—, —CH═N—, —C(C═O)O—, —(C═O)NH—, —(C═O)—, —O(C═O)NR₅—, —O(C═S)NR₅—, —O(C═O)O—, —O(C═O)—, —NH(C═O)O—, —NH(C═O)—, —NH(C═NH)—, —NH(C═O)NR₅—, —NH(C═NH)NR₅—, —NH(C═S)NR₅—, —NH(C═S)—, —OCH₂(C═O)NR₅—, —R₅OR₆C(═O)NH—, —R₅NH(C═O)—, —R₅N—, —NH(SO₂)—, —NH(SO₂)NR₅—, —OR₆—, —NHR₆—, —SO₂—, and —SR₆—;
R₂, R₃, X₂, and U₁ are each independently selected from OH, halogen, nitrile, nitro, optionally substituted amine, optionally substituted imine, optionally substituted C₁-C₂₀ alkamino, optionally substituted sulfhydryl, optionally substituted carboxyl, optionally substituted cyano, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;
each X₃ is independently selected from optionally substituted C₁-C₂₀ alkyl, optionally substituted C₃-C₂₀ cycloalkyl, optionally substituted C₂-C₂₀ heterocycloalkyl; optionally substituted C₅-C₁₅ aryl, and optionally substituted C₁-C₁₅ heteroaryl;
U₂ is a substituent of the ring nitrogen atom and is selected from optionally substituted C₁-C₂₀ alkyl, optionally substituted C₃-C₂₀ cycloalkyl, optionally substituted C₂-C₂₀ heterocycloalkyl, optionally substituted C₅-C₁₅ aryl, optionally substituted C₃-C₁₅ heteroaryl, and a bond;
U₃ is a substituent of ring nitrogen atom and is selected from H, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy, optionally substituted C₁-C₂₀ alkamino, optionally substituted carboxyl, optionally substituted cyano;
Ar is selected from optionally substituted C₃-C₂₀ cycloalkyl, optionally substituted C₂-C₂₀ heterocycloalkyl, optionally substituted C₅-C₁₅ aryl, and optionally substituted C₁-C₁₅ heteroaryl;
R₅ and R₆ are each independently selected from H, optionally substituted C₁-C₂₀ alkyl, optionally substituted C₃-C₂₀ cycloalkyl, optionally substituted C₂-C₂₀ heterocycloalkyl; optionally substituted C₅-C₁₅ aryl, and optionally substituted C₂-C₁₅ heteroaryl;
b and g are each independently 0, 1, 2, or 3;
n is 1 or 2;
each E independently comprises an Fc domain monomer, and albumin protein, an albumin protein-binding peptide, or an Fc-binding peptide;
L is a linker covalently attached to E and to each Y of each of A₁ or A₁ and A₂; and
T is an integer from 1 to 20;
each squiggly line in formulas (D-I), (M-I), (1), and (2) indicates a covalent linkage between each L and each E;
or a pharmaceutically acceptable salt thereof.

2. The conjugate of claim 1, wherein the conjugate is described by formula (D-I): 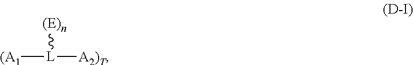
wherein each A₁ and each A₂ is independently selected from any one of formulas (A-I)-(A-III);
each E independently comprises an Fc domain monomer;
n is 1 or 2;
T is an integer from 1 to 20; and
the squiggly line connected to the E indicates that each A₁-L-A₂ is covalently attached to E;
or a pharmaceutically acceptable salt thereof.

3. The conjugate of claim 2, wherein the conjugate is described by formula (D-II): 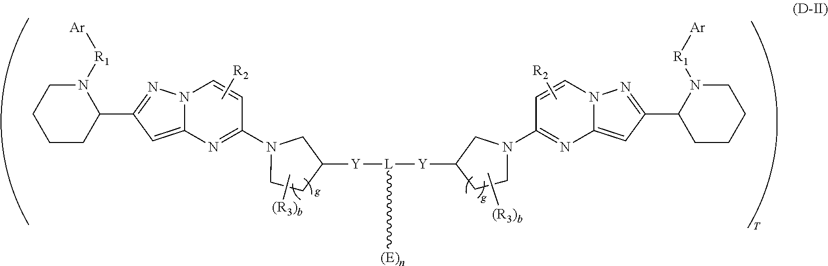
or a pharmaceutically acceptable salt thereof.

4. The conjugate of claim 3, wherein the conjugate is described by formula (D-II-1): 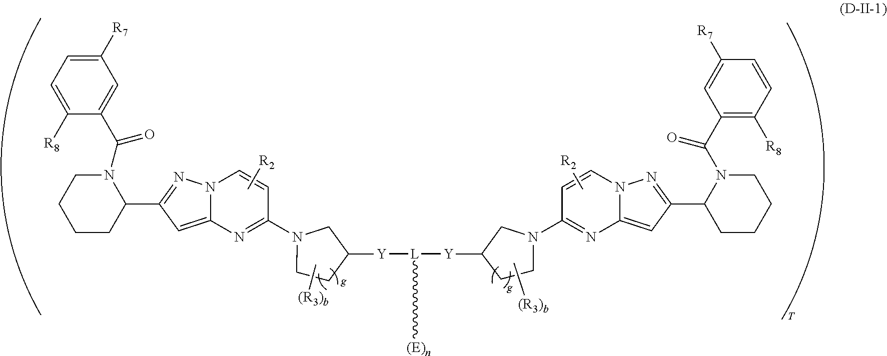
wherein R₇ and R₈ are each independently selected from OH, halogen, nitrile, nitro, optionally substituted amine, optionally substituted imine, optionally substituted C₁-C₂₀ alkamino, optionally substituted sulfhydryl, optionally substituted carboxyl, optionally substituted cyano, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;
or a pharmaceutically acceptable salt thereof.

5. The conjugate of claim 4, wherein the conjugate is described by formula (D-II-2): 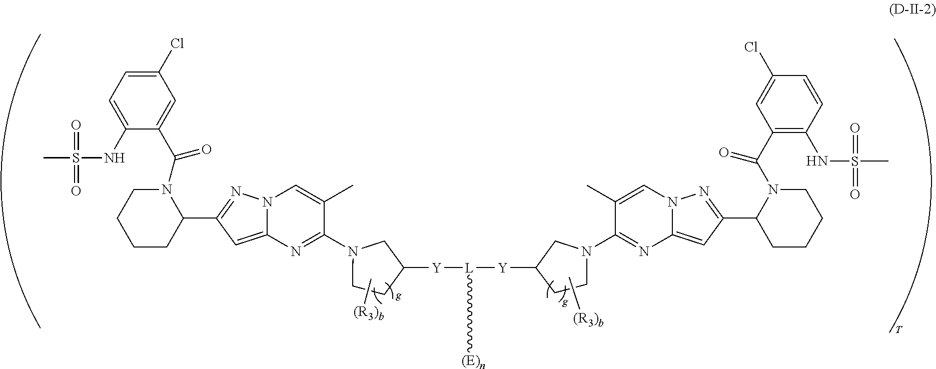
or a pharmaceutically acceptable salt thereof.

6. The conjugate of claim 5, wherein the conjugate is described by the formula (D-II-3) 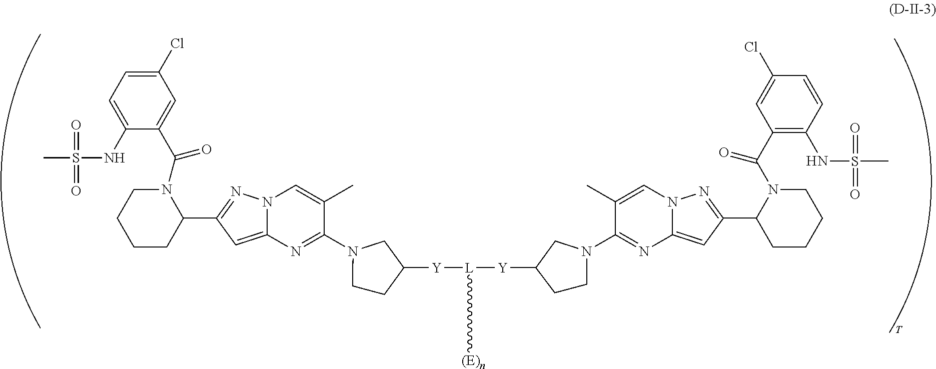
or a pharmaceutically acceptable salt thereof.

7. The conjugate of claim 6, wherein the conjugate is described by the formula (D-II-4): 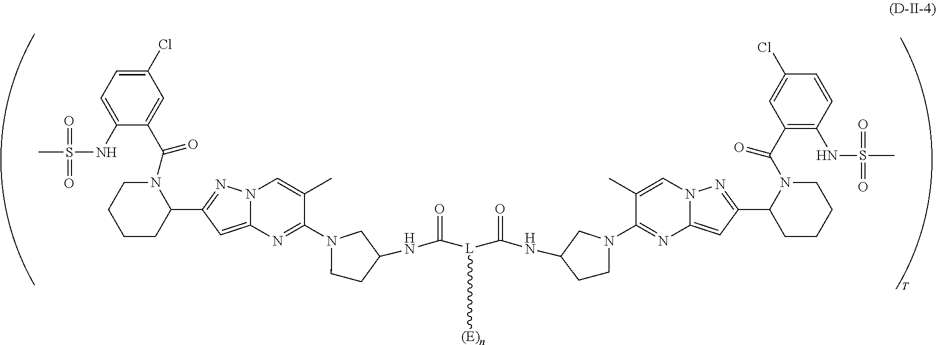
or a pharmaceutically acceptable salt thereof.

8. The conjugate of claim 7, wherein the conjugate is described by the formula (D-II-5): 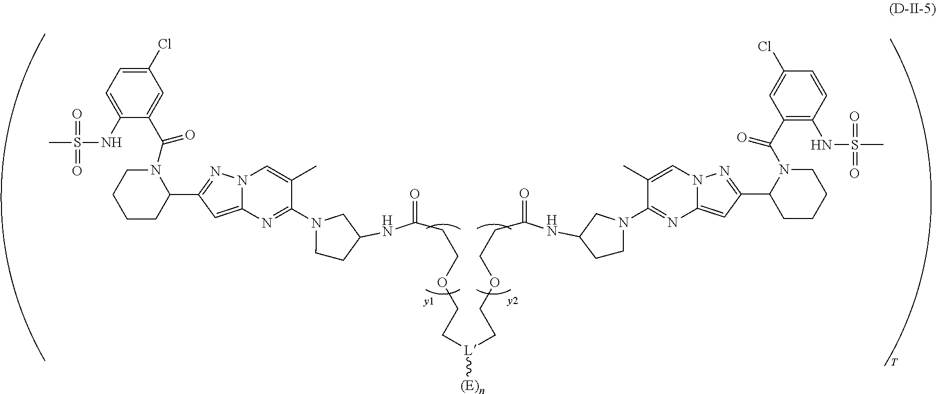
wherein L′ is the remainder of L, and
y₁ and y₂ are each independently an integer from 1-20,
or a pharmaceutically acceptable salt thereof.

9. The conjugate of claim 6, wherein the conjugate is described by formula (D-II-6): 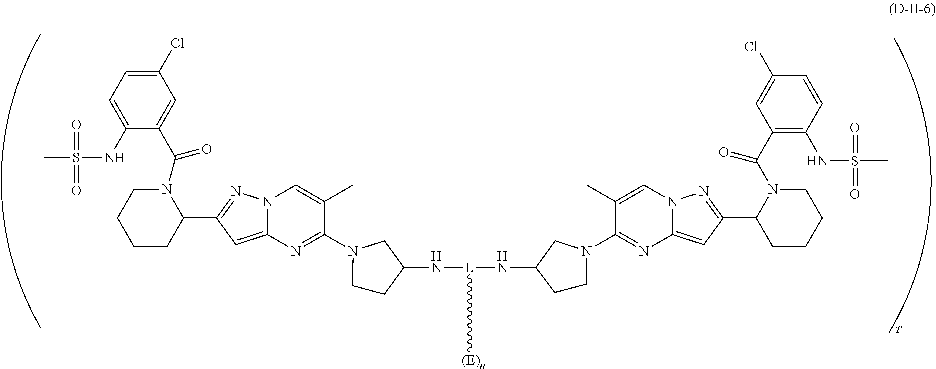
or a pharmaceutically acceptable salt thereof.

10. The conjugate of claim 9, wherein the conjugate is described by the formula (D-II-7): 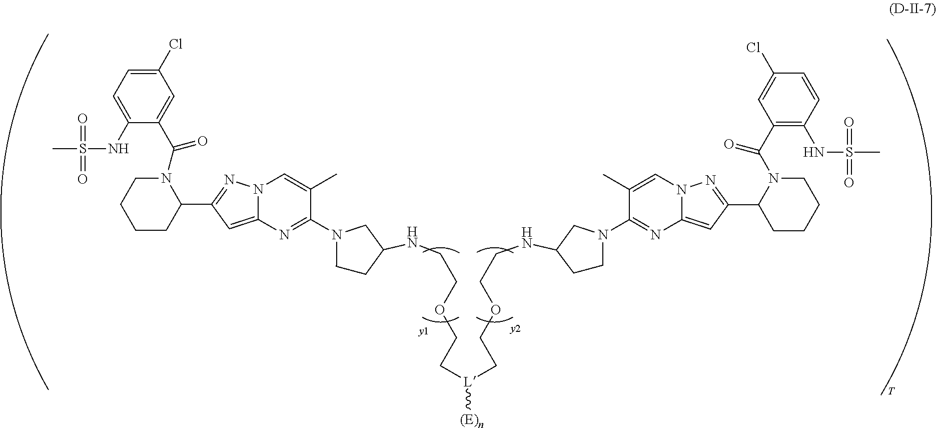
wherein L′ is the remainder of L, and
y₁ and y₂ are each independently an integer from 1-20,
or a pharmaceutically acceptable salt thereof.

11. The conjugate of 6, wherein the conjugate is described by formula (D-II-8): 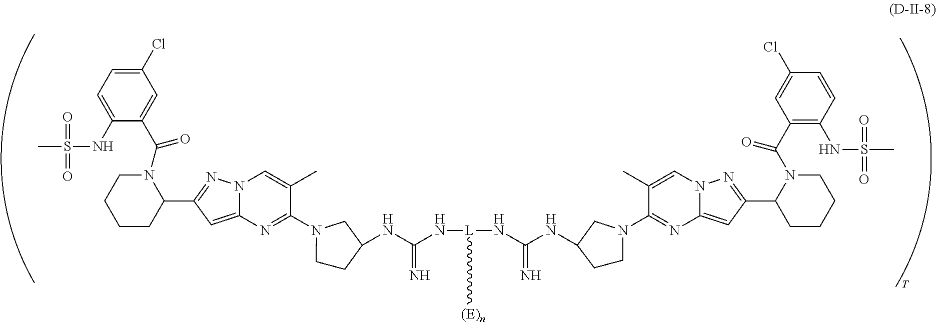
or a pharmaceutically acceptable salt thereof.

12. The conjugate of claim 11, wherein the conjugate is described by formula (D-II-9): 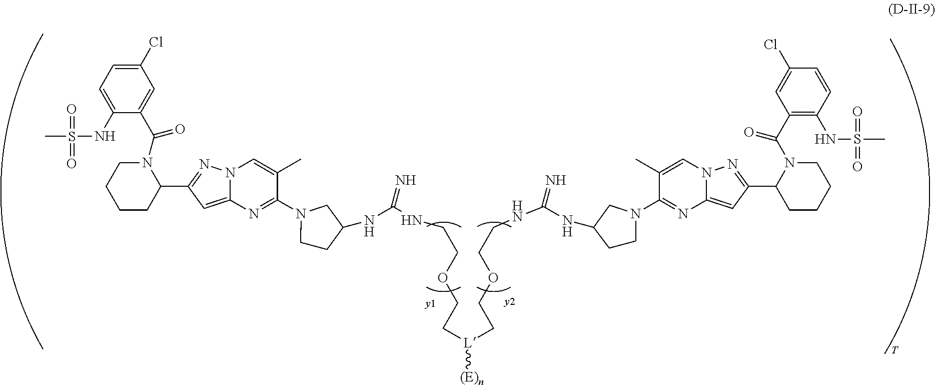
wherein L′ is the remainder of L, and
y₁ and y₂ are each independently an integer from 1-20,
or a pharmaceutically acceptable salt thereof.

13. The conjugate of claim 5, wherein the conjugate is described by the formula (D-II-10): 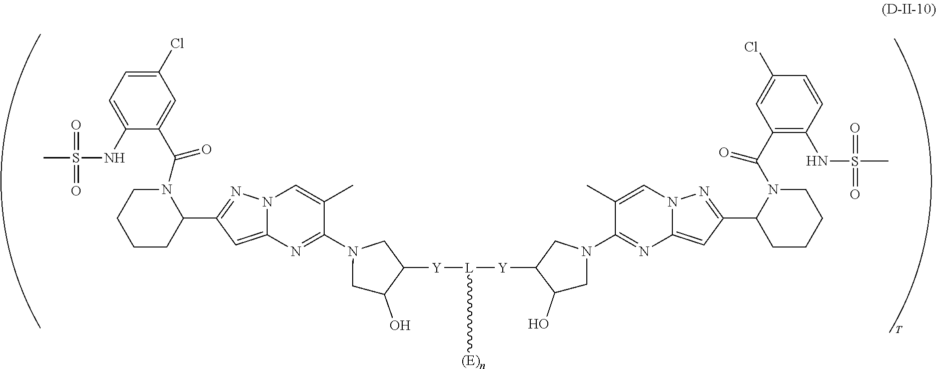
or a pharmaceutical acceptable salt thereof.

14. The conjugate of claim 13, wherein the conjugate is described by formula (D-II-11): 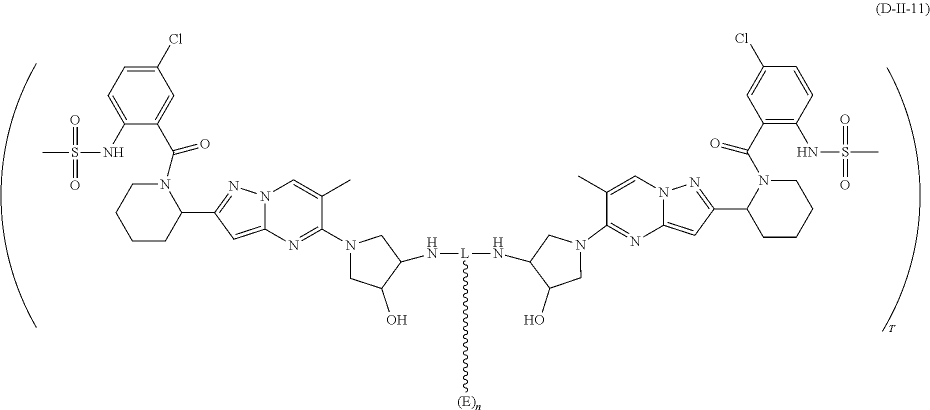
or a pharmaceutically acceptable salt thereof.

15. The conjugate of claim 14, wherein the conjugate is described by the formula (D-II-12): 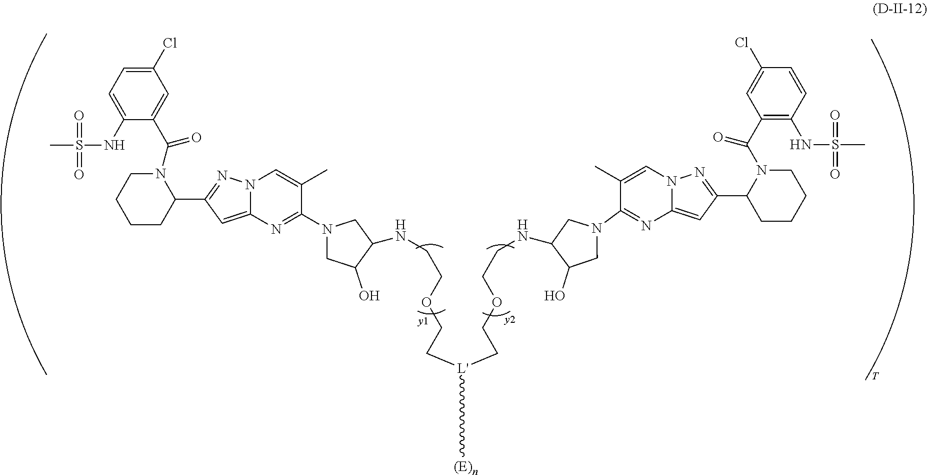
wherein L′ is the remainder of L, and
y₁ and y₂ are each independently an integer from 1-20,
or a pharmaceutically acceptable salt thereof.

16. The conjugate of claim 2, wherein the conjugate is described by formula (D-II-13): 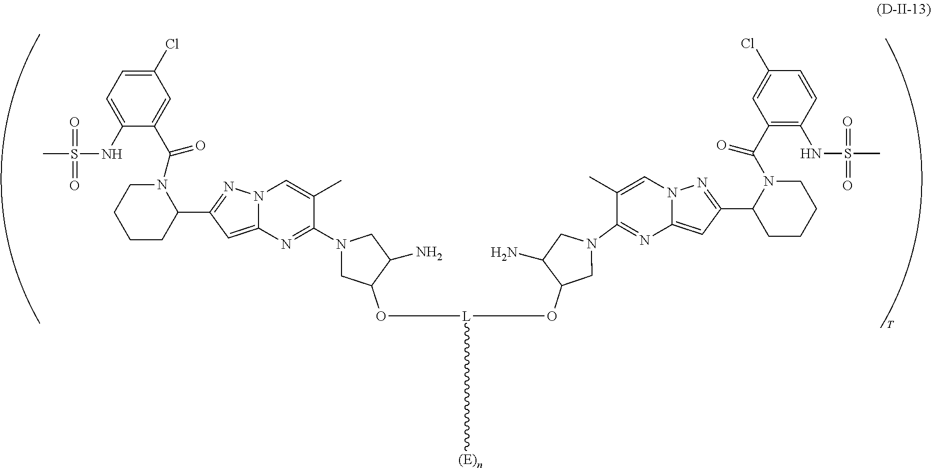
or a pharmaceutically acceptable salt thereof.

17. The conjugate of claim 16, wherein the conjugate is described by formula (D-II-14): 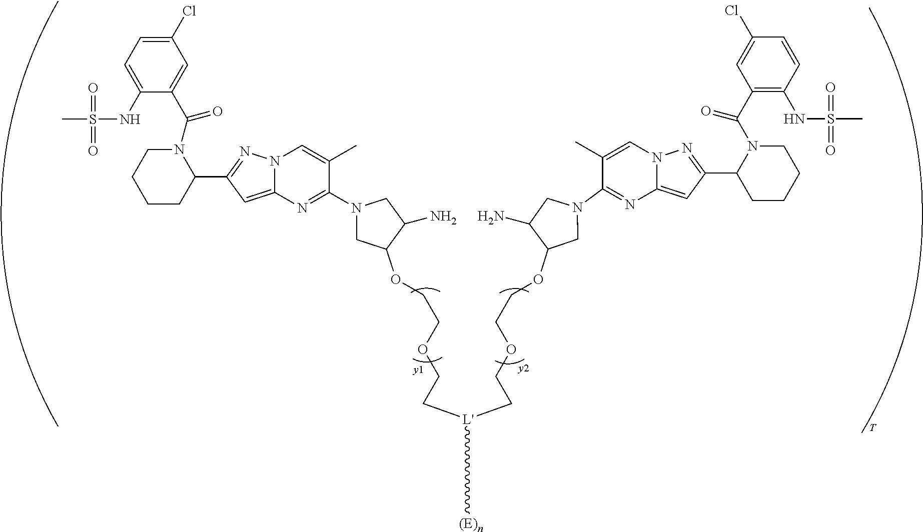
wherein L′ is the remainder of L, and
y₁ and y₂ are each independently an integer from 1-20,
or a pharmaceutically acceptable salt thereof.

18. The conjugate of claim 5, wherein the conjugate is described by formula (D-II-15): 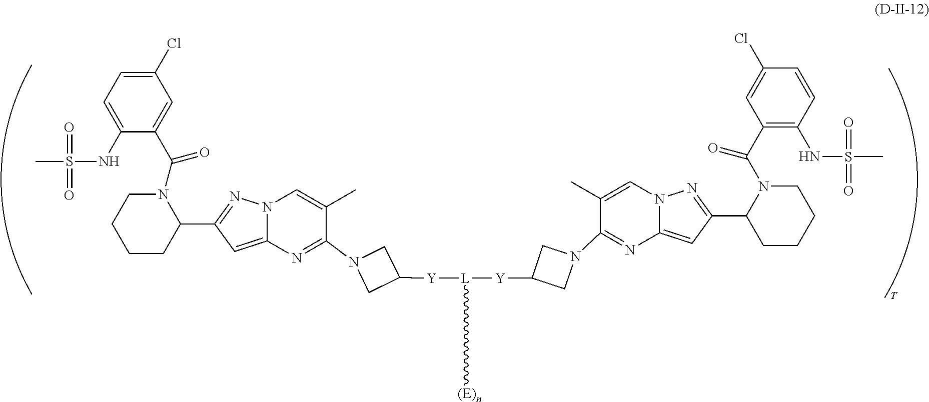
or a pharmaceutically acceptable salt thereof.

19. The conjugate of claim 18, wherein the conjugate is described by formula (D-II-16): 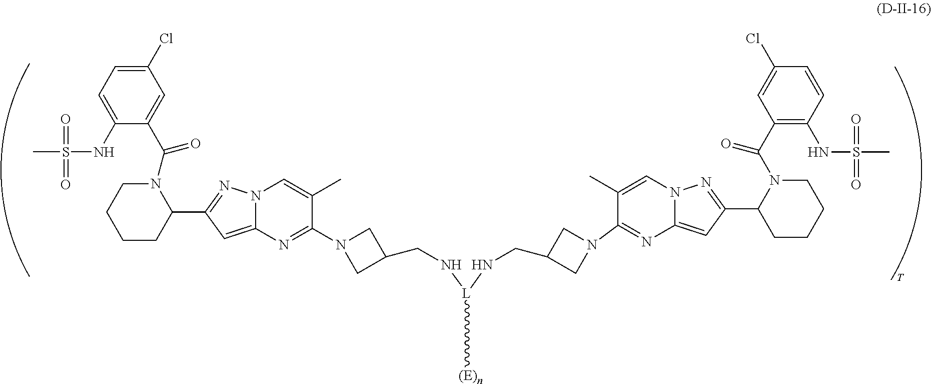
or a pharmaceutically acceptable salt thereof.

20. The conjugate of claim 19, wherein the conjugate is described by formula (D-II-17): 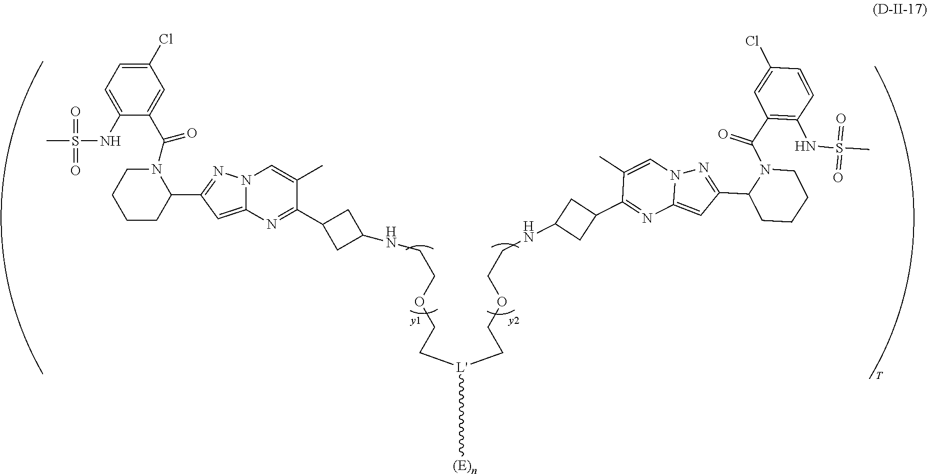
wherein L′ is the remainder of L, and
y₁ and y₂ are each independently an integer from 1-20,
or a pharmaceutically acceptable salt thereof.

21. The conjugate of claim 2, wherein the conjugate is described by formula (D-III): 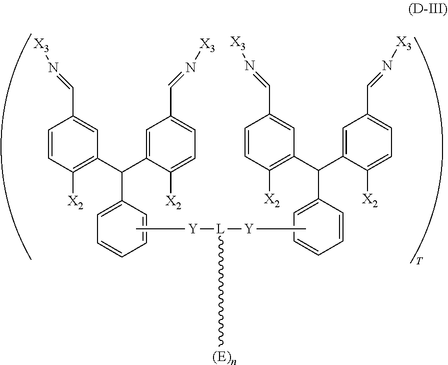
or a pharmaceutically acceptable salt thereof.

22. The conjugate of claim 21, wherein the conjugate is described by formula (D-III-1): 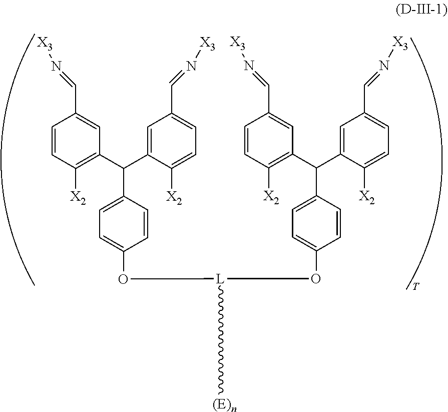
or a pharmaceutically acceptable salt thereof.

23. The conjugate of claim 22, wherein the conjugate is described by formula (D-III-2): 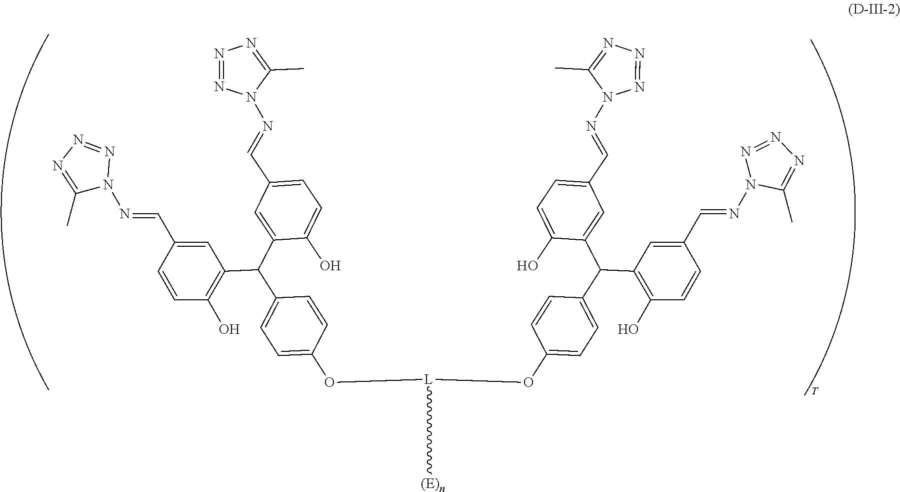
or a pharmaceutically acceptable salt thereof.

24. The conjugate of claim 23, wherein the conjugate is described by formula (D-III-3): 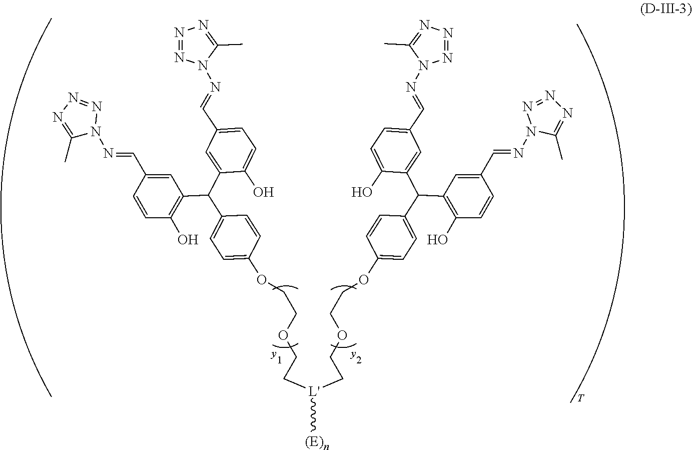
wherein L′ is the remainder of L, and
y₁ and y₂ are each independently an integer from 1-20,
or a pharmaceutically acceptable salt thereof.

25. The conjugate of claim 2, wherein the conjugate is described by formula (D-IV): 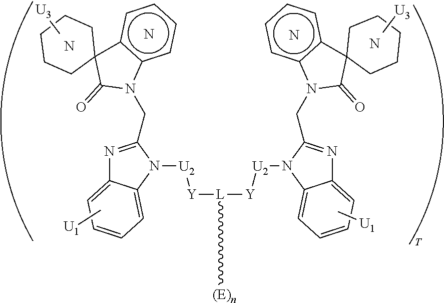
wherein U₂ is an optionally substituted C₁-C₈ alkyl,
or a pharmaceutically acceptable salt thereof.

26. The conjugate of claim 25, wherein the conjugate is described by formula (D-IV-1): 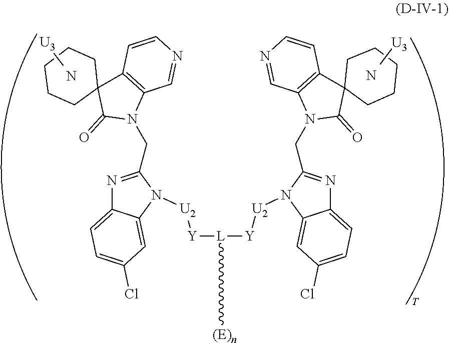
or a pharmaceutically acceptable salt thereof.

27. The conjugate of claim 26, wherein the conjugate is described by formula (D-IV-2): 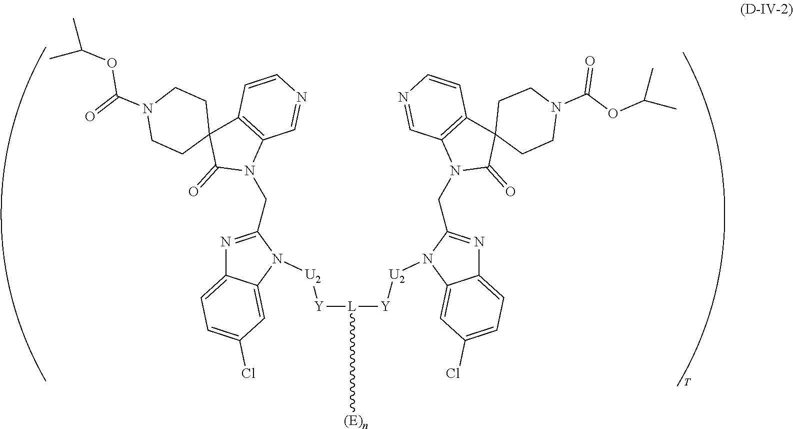
or a pharmaceutically acceptable salt thereof.

28. The conjugate of claim 27, wherein the conjugate is described by formula (D-IV-3): 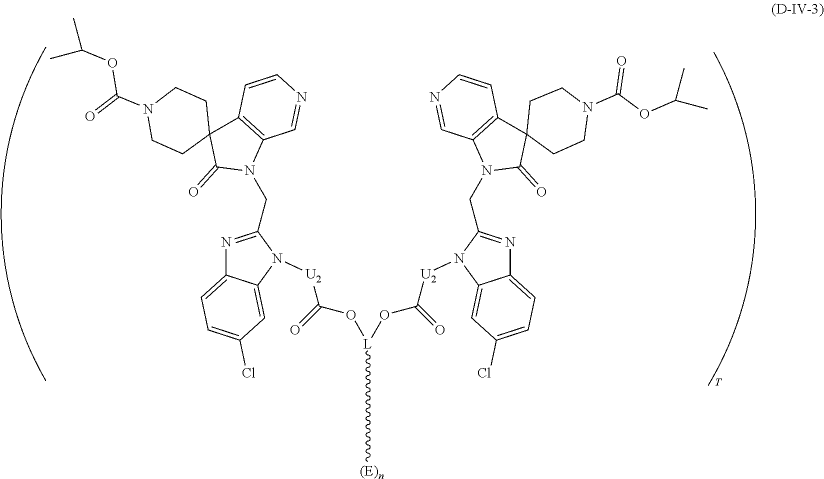
or a pharmaceutically acceptable salt thereof.

29. The conjugate of claim 28, wherein the conjugate is described by formula (D-IV-4): 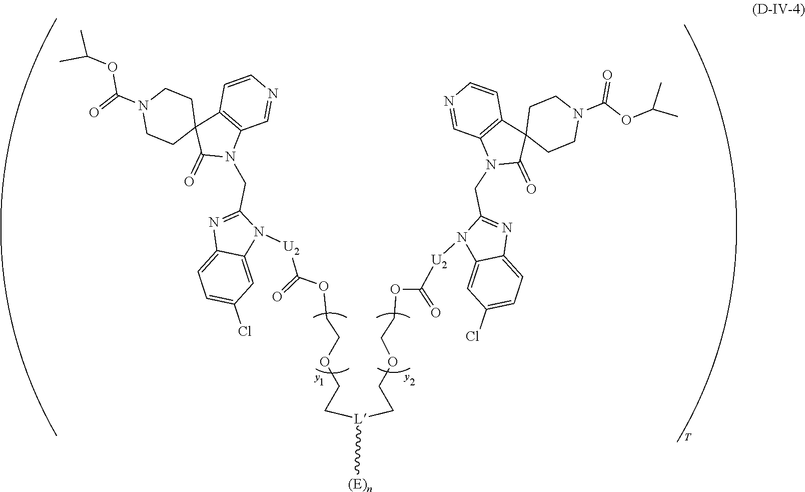
wherein L′ is the remainder of L, and
y₁ and y₂ are each independently an integer from 1-20,
or a pharmaceutically acceptable salt thereof.

30. The conjugate of claim 27, wherein the conjugate is described by formula (D-IV-5): 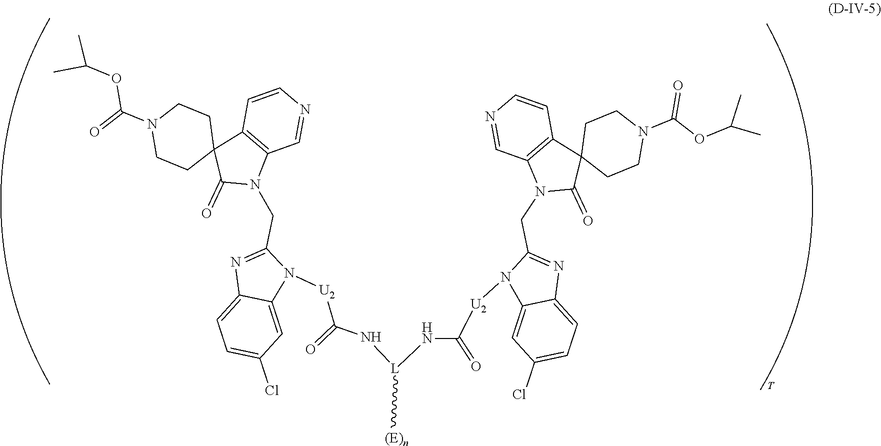
or a pharmaceutically acceptable salt thereof.

31. The conjugate of claim 30, wherein the conjugate is described by formula (D-IV-6): 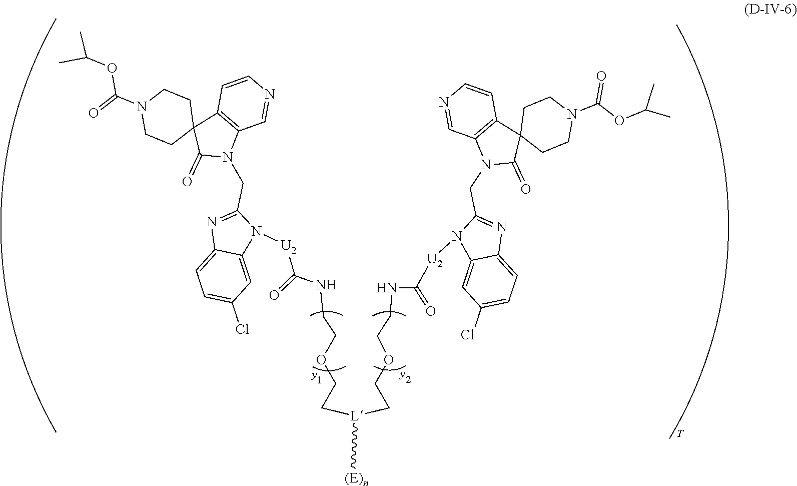
wherein L′ is the remainder of L, and
y₁ and y₂ are each independently an integer from 1-20,
or a pharmaceutically acceptable salt thereof.

32. The conjugate of claim 27, wherein the conjugate is described by formula (D-IV-7): 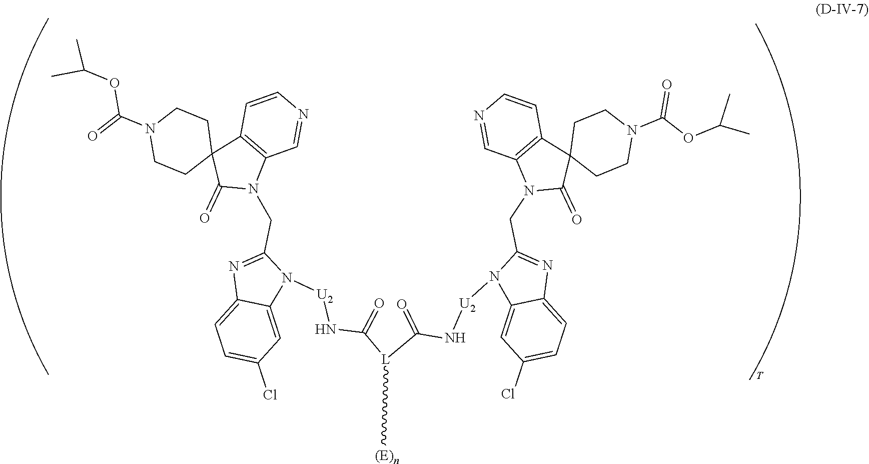
or a pharmaceutically acceptable salt thereof.

33. The conjugate of claim 32, wherein the conjugate is described by formula (D-IV-8): 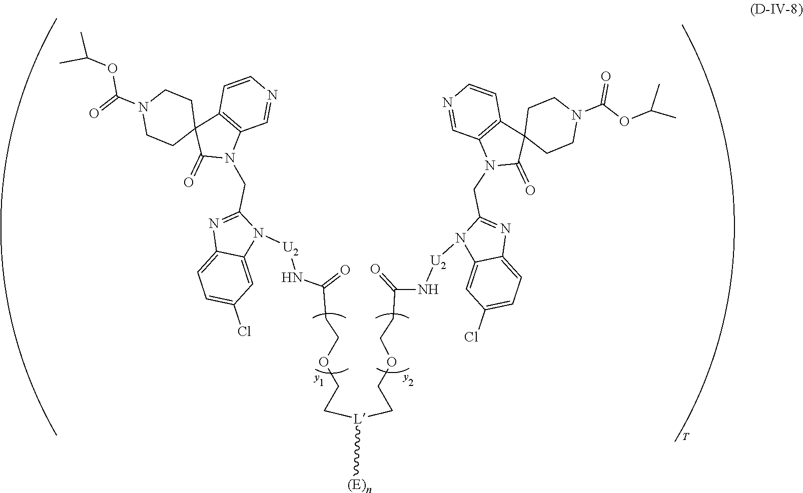
wherein L′ is the remainder of L, and
y₁ and y₂ are each independently an integer from 1-20,
or a pharmaceutically acceptable salt thereof.

34. The conjugate of claim 25, wherein the conjugate is described by formula (D-IV-9): 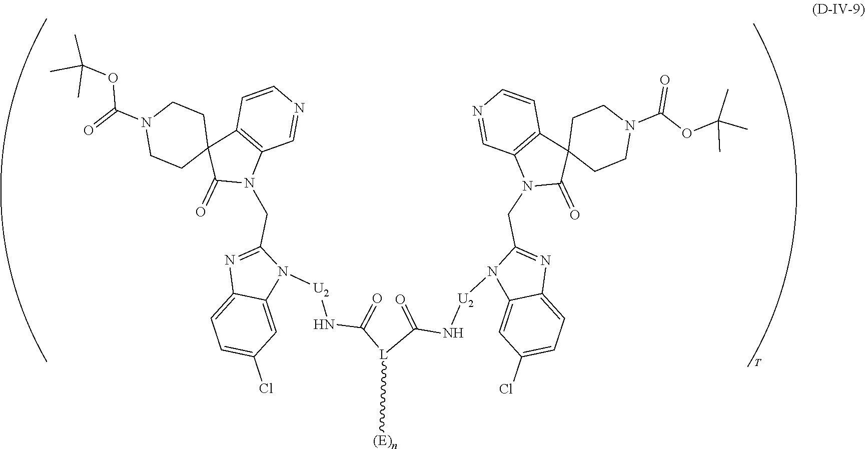
or a pharmaceutically acceptable salt thereof.

35. The conjugate of claim 34, wherein the conjugate is described by formula (D-IV-10): 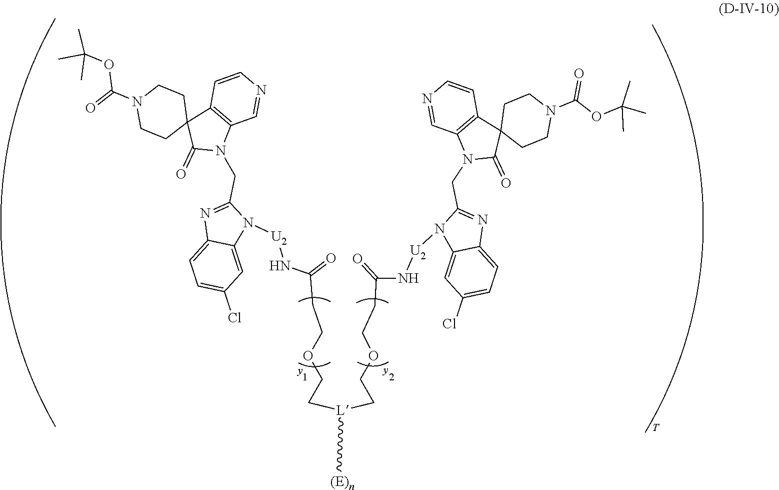
wherein L′ is the remainder of L, and
y₁ and y₂ are each independently an integer from 1-20,
or a pharmaceutically acceptable salt thereof.

36. The conjugate of claim 27, wherein the conjugate is described by formula (D-IV-11): 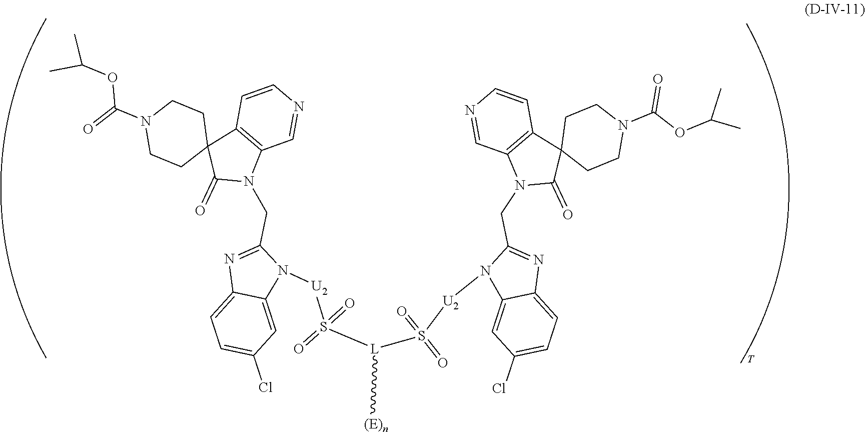
or a pharmaceutically acceptable salt thereof.

37. The conjugate of claim 36, wherein the conjugate is described by formula (D-IV-12): 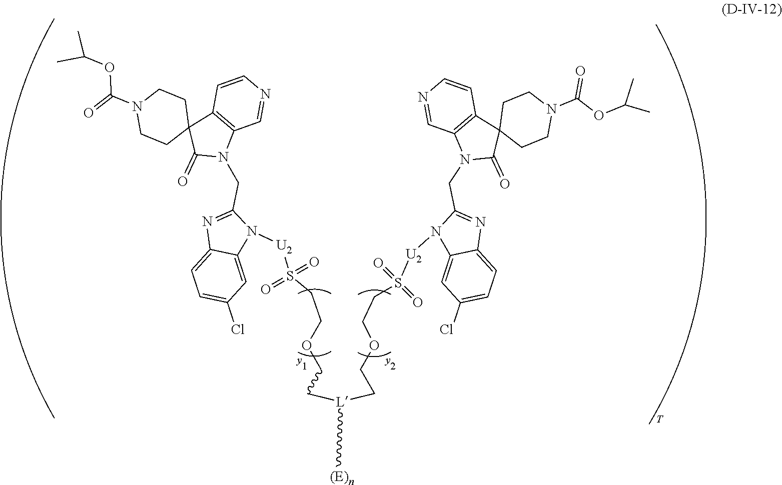
wherein L′ is the remainder of L, and
y₁ and y₂ are each independently an integer from 1-20,
or a pharmaceutically acceptable salt thereof.

38. The conjugate of claim 26, wherein the conjugate is described by formula (D-IV-13): 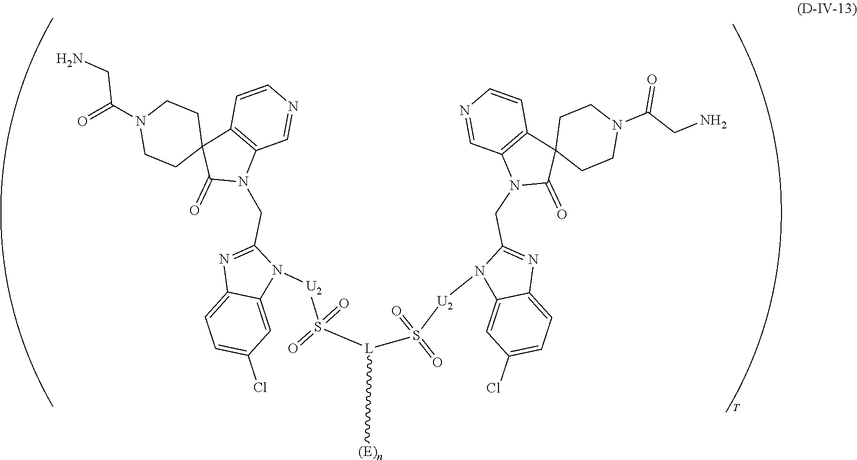
or a pharmaceutically acceptable salt thereof.

39. The conjugate of claim 38, wherein the conjugate is described by formula (D-IV-14): 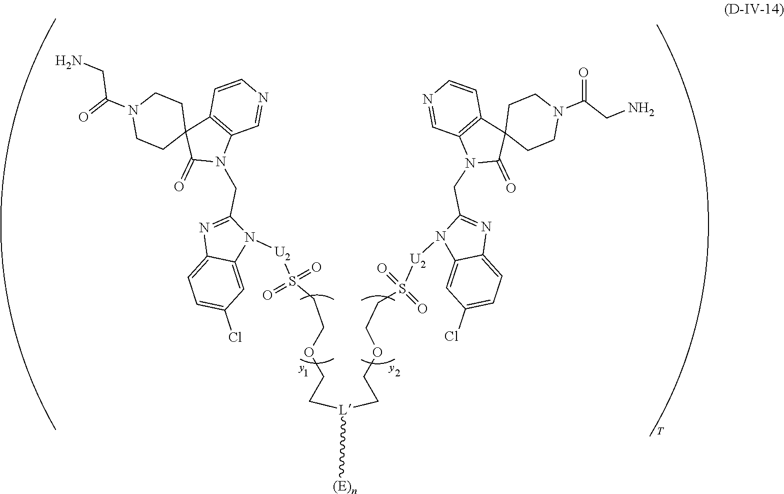
wherein L′ is the remainder of L, and
y₁ and y₂ are each independently an integer from 1-20,
or a pharmaceutically acceptable salt thereof.

40. The conjugate of claim 26, wherein the conjugate is described by formula (D-IV-15): 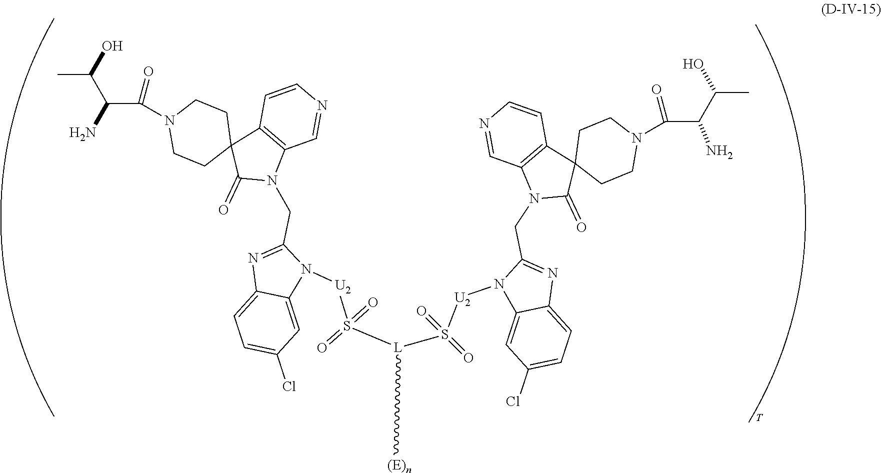
or a pharmaceutically acceptable salt thereof.

41. The conjugate of claim 40, wherein the conjugate is described by formula (D-IV-16): 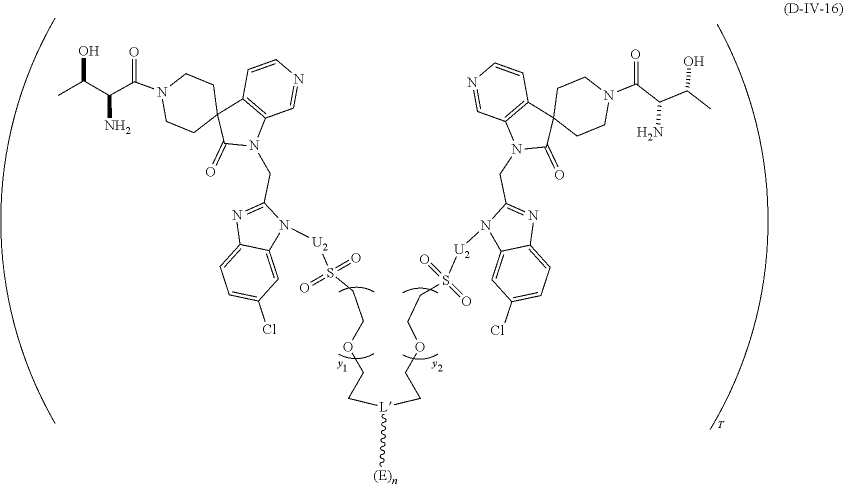
wherein L′ is the remainder of L, and
y₁ and y₂ are each independently an integer from 1-20,
or a pharmaceutically acceptable salt thereof.

42. The conjugate of claim 26, wherein the conjugate is described by formula (D-IV-17): 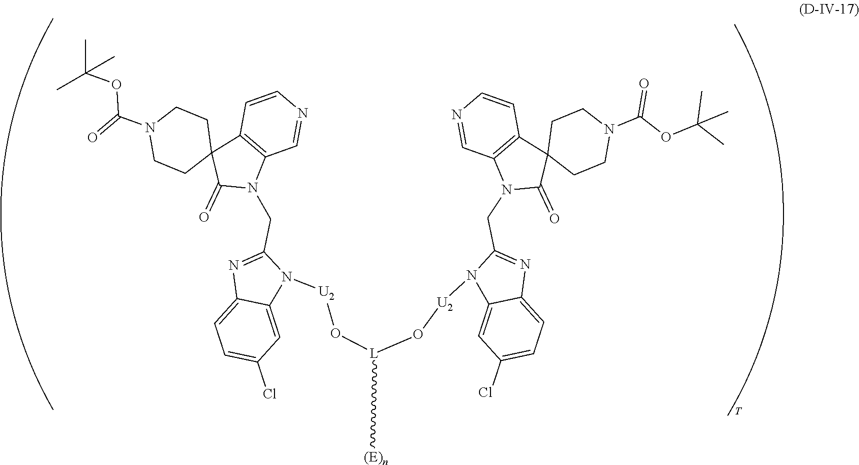
or a pharmaceutically acceptable salt thereof.

43. The conjugate of claim 42, wherein the conjugate is described by formula (D-IV-18): 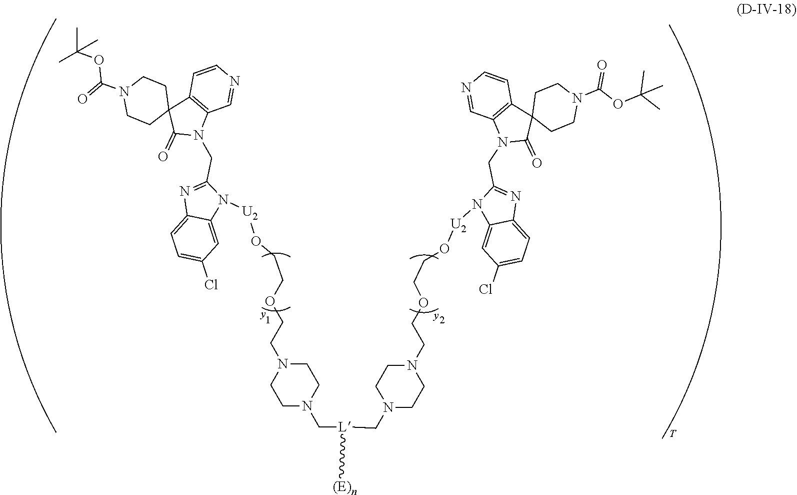
wherein L′ is the remainder of L, and
y₁ and y₂ are each independently an integer from 1-20,
or a pharmaceutically acceptable salt thereof.

44. The conjugate of any one of claims 26-43, wherein U₂ is C₂-C₆ alkyl.

45. The conjugate of any one of claims 1-44, wherein L or L′ comprises one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino,
wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

46. The conjugate of claim 45, wherein the backbone of L or L′ consists of one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino,
wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

47. The conjugate of claim 45 or 46, wherein L is oxo substituted.

48. The conjugate of any one of claims 1-47, wherein the backbone of L or L′ comprises no more than 250 atoms.

49. The conjugate of any one of claims 1-48, wherein L or L′ is capable of forming an amide, a carbamate, a sulfonyl, or a urea linkage.

50. The conjugate of any one of claims 1-44, wherein L or L′ is a bond.

51. The conjugate of any one of claims 1-44, wherein L or L′ is an atom.

52. The conjugate of any one of claims 1-51, wherein each L is described by formula (D-L-I): 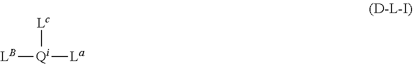
wherein
L^{A} is described by formula G^{A1}-(Z^{A1})_{g1}—(Y^{A1})ₕ₁—(Z^{A2})ᵢ₁—(Y^{A2})ⱼ₁—(Z^{A3})ₖ₁—(Y^{A3})ₗ₁—(Z^{A4})ₘ₁—(Y^{A4})ₙ₁—(Z^{A5})_{O1}-G^{A2};
L^{B} is described by formula G^{B1}-(Z^{B1})_{g2}—(Y^{B1})ₕ₂—(Z^{B2})ᵢ₂—(Y^{B2})ⱼ₂—(Z^{B3})ₖ₂—(Y^{B3})ₗ₂—(Z^{B4})ₘ₂—(Y^{B4})ₙ₂—(Z^{B5})_{O2}-G^{B2};
L^{C} is described by formula G^{C1}-(Z^{C1})_{g3}—(Y^{C1})ₕ₃—(Z^{C2})ᵢ₃—(Y^{C2})ⱼ₃—(Z^{C3})ₖ₃—(Y^{C3})ₗ₃—(Z^{C4})ₘ₃—(Y^{C4})ₙ₃—(Z^{C5})_{O3}-G^{C2};
G^{A1} is a bond attached to Qⁱ;
G^{A2} is a bond attached to A1;
G^{B1} is a bond attached to Qⁱ;
G^{B2} is a bond attached to A2;
G^{C1} is a bond attached to Qⁱ;
G² is a bond attached to E;
each of Z^{A1}, Z^{A2}, Z^{A3}, Z^{A4}, Z^{A5}, Z^{B1}, Z^{B2}, Z^{B3}, Z^{B4}, Z^{B5}, Z^{C1}, Z^{C2}, Z^{C3}, Z^{C4}, and Z^{C5} is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene;
each of Y^{A1}, Y^{A2}, Y^{A3}, Y^{A4}, Y^{B1} Y^{B2}, Y^{B3}, Y^{B4}, Y^{C1}, Y^{C2} Y^{C3}, and Y^{C4} is, independently, O, S, NRⁱ, P, carbonyl, thiocarbonyl, sulfonyl, phosphate, phosphoryl, or imino;
Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl;
each of g1, h1, i1, j1, k1, l1, m1, n1, o1, g2, h2, i2, j2, k2, l2, m2, n2, o2, g3, h3, i3, j3, k3, l3, m3, n3, and o3 is, independently, 0 or 1;
Qⁱ is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

53. The conjugate of claim 52, wherein L is selected from 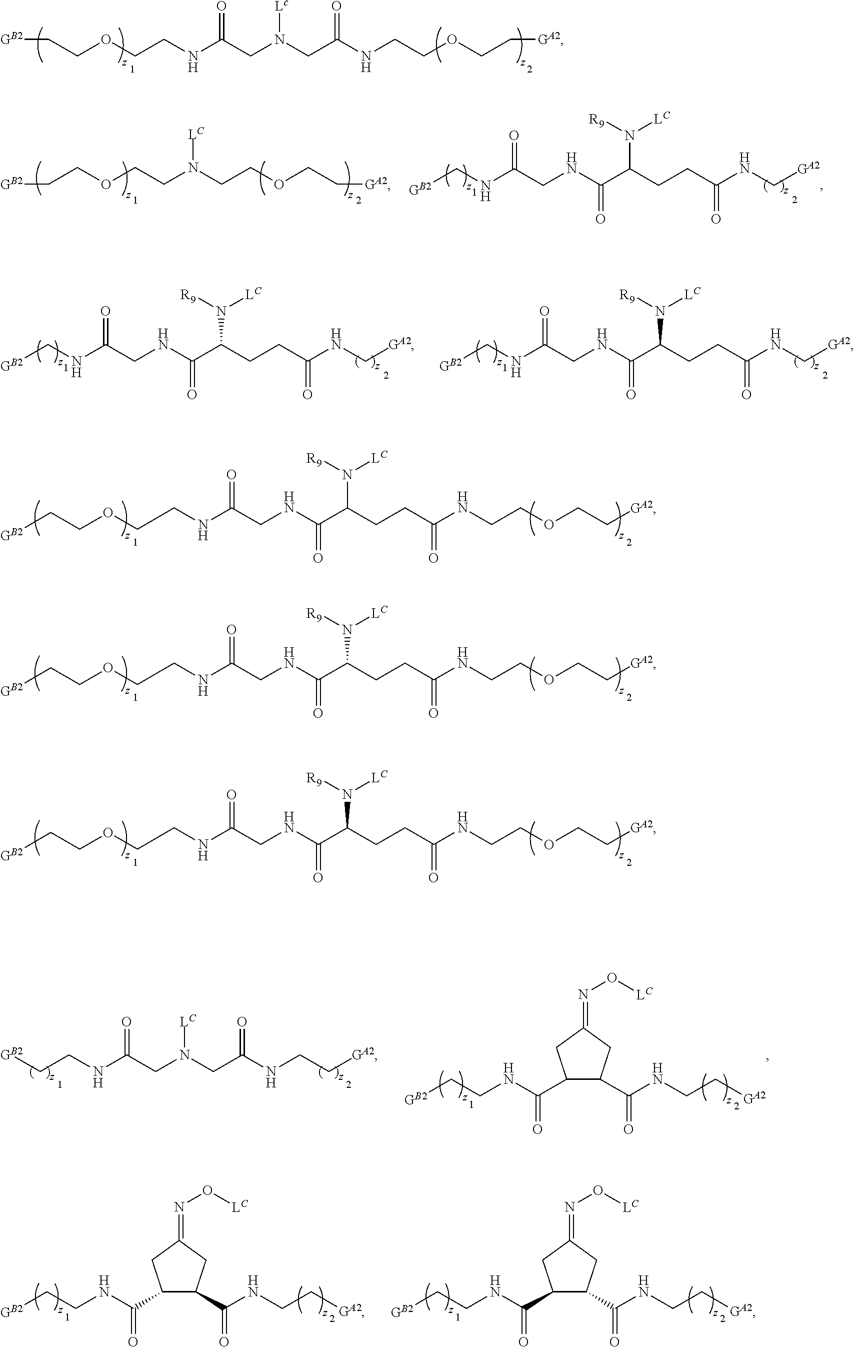 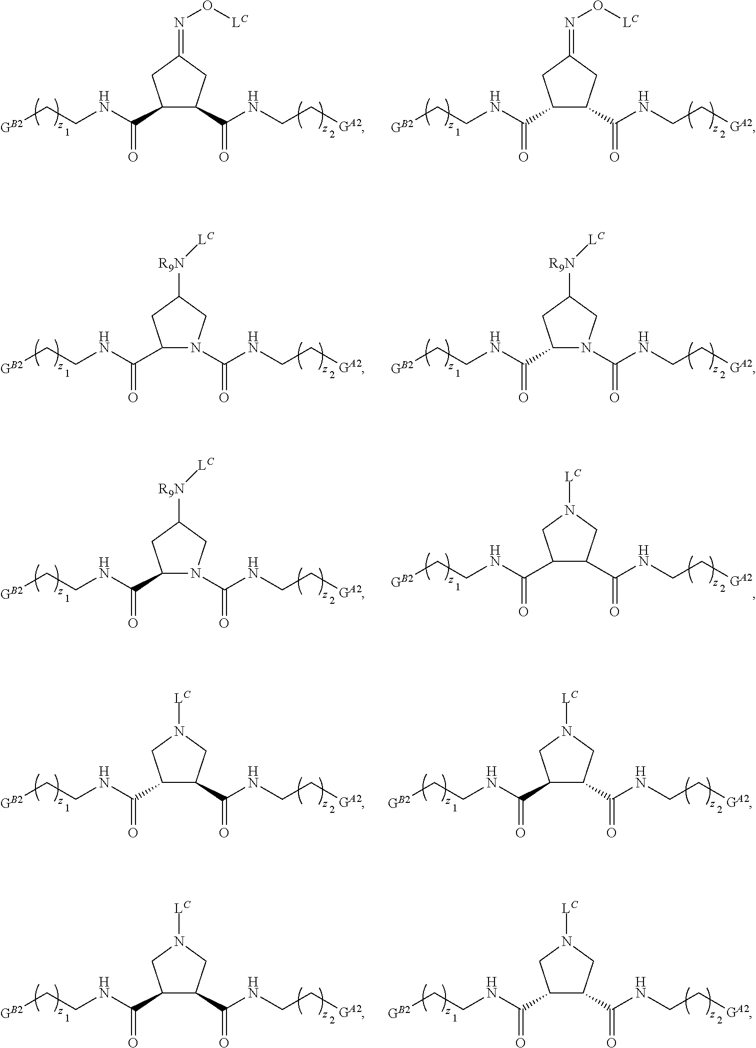 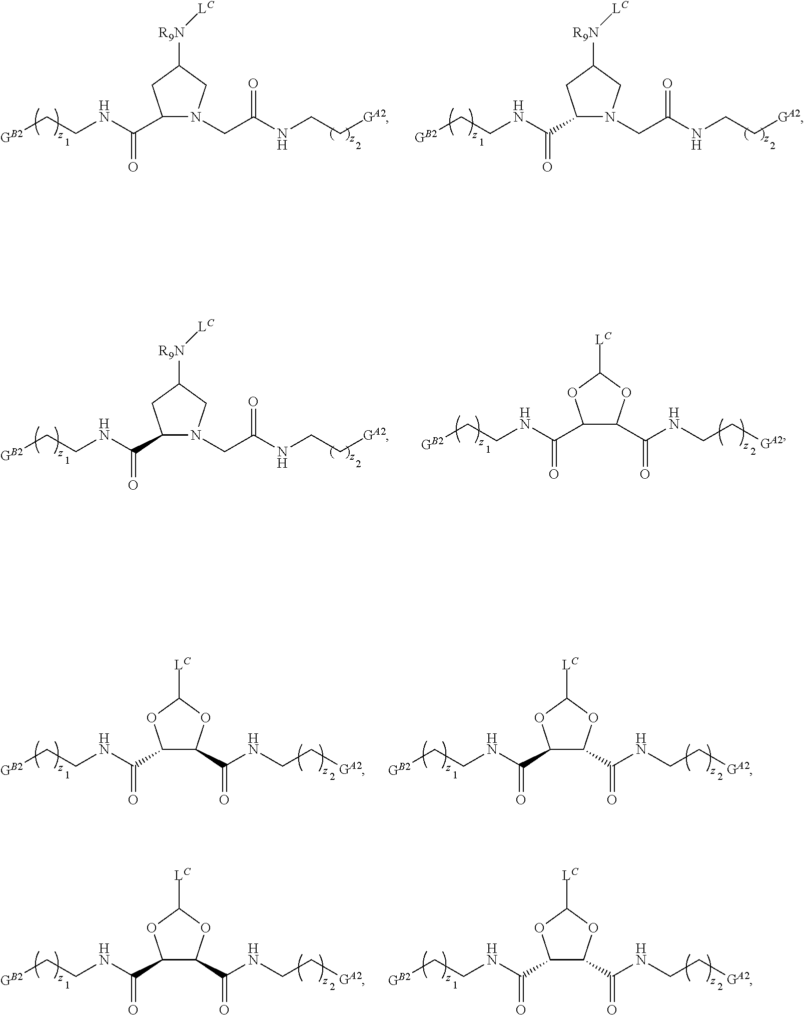 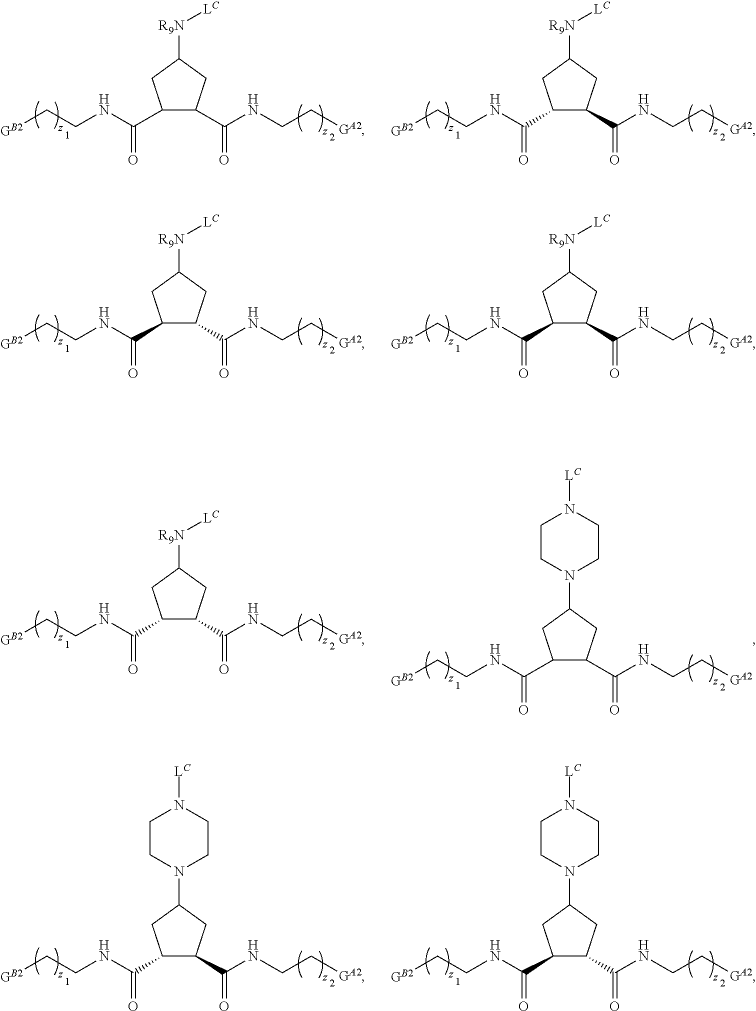 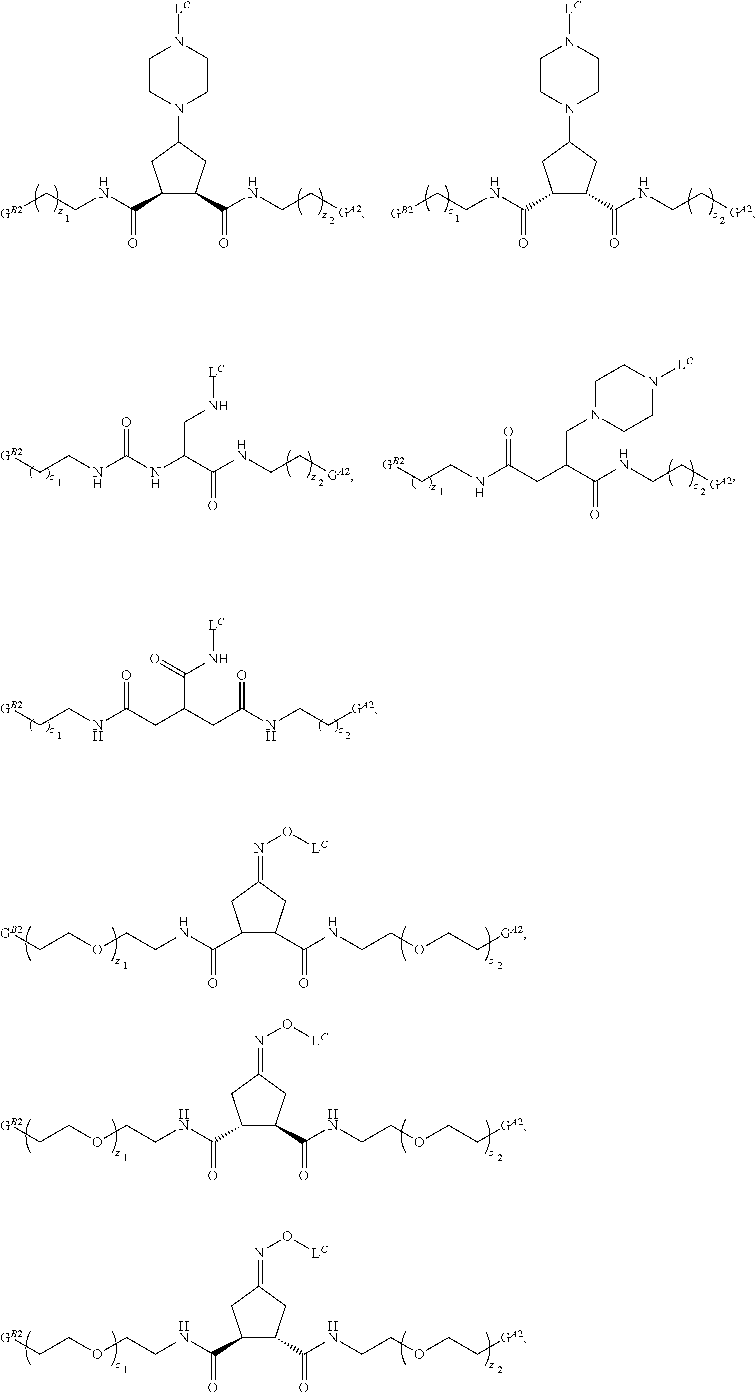 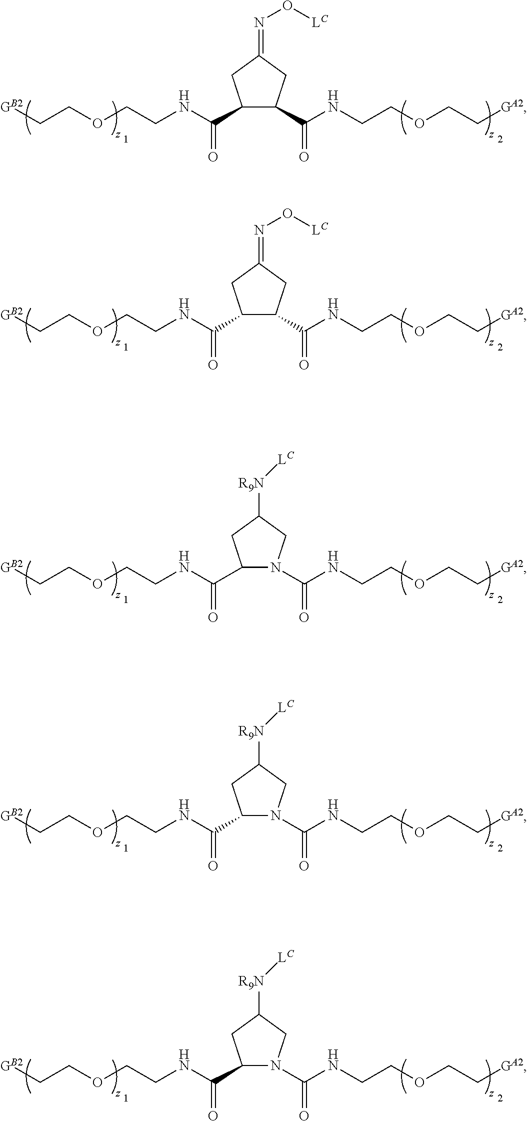 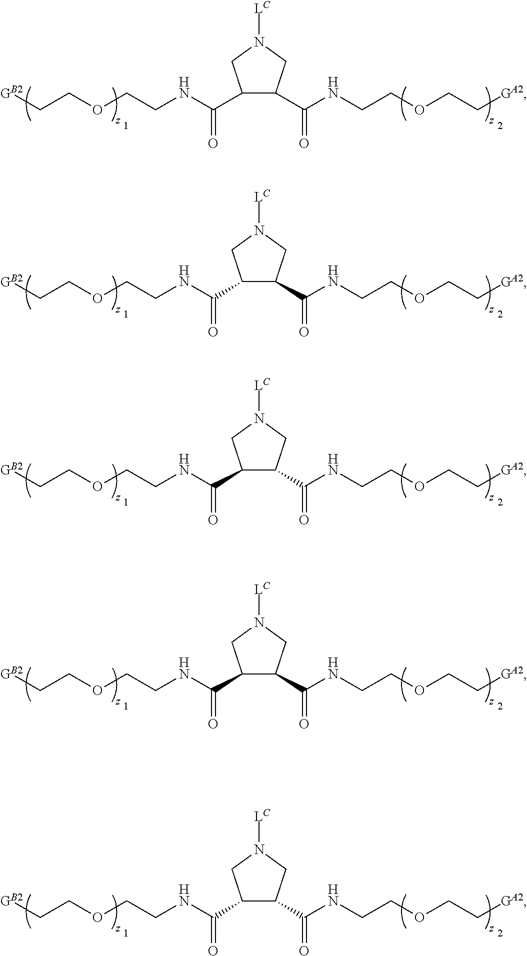 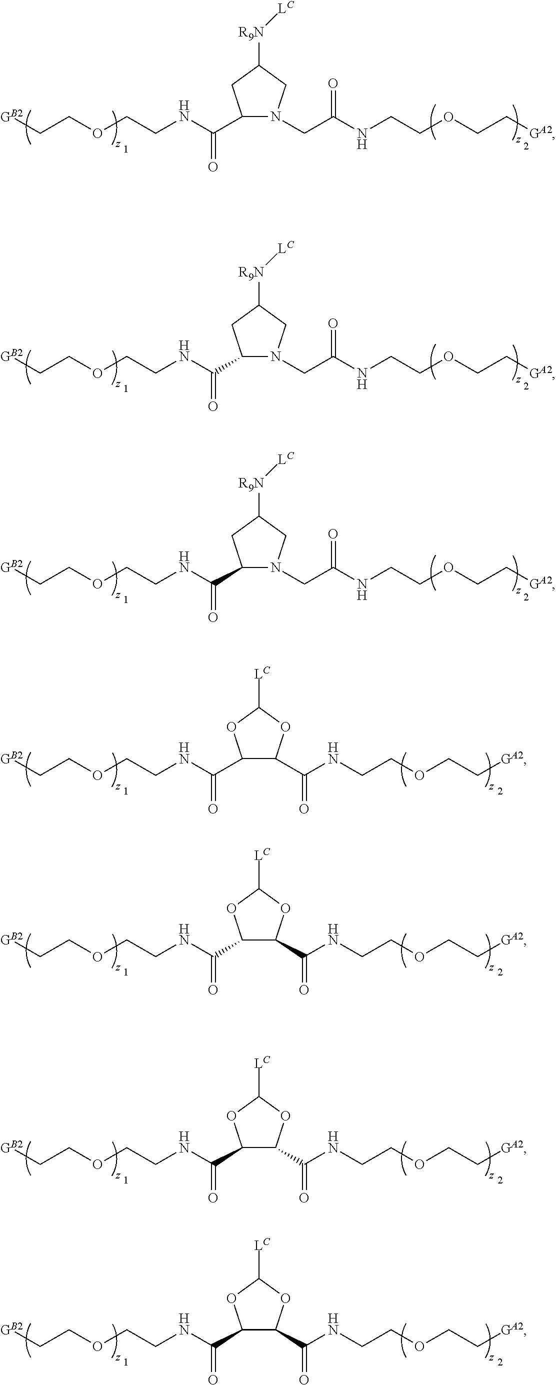 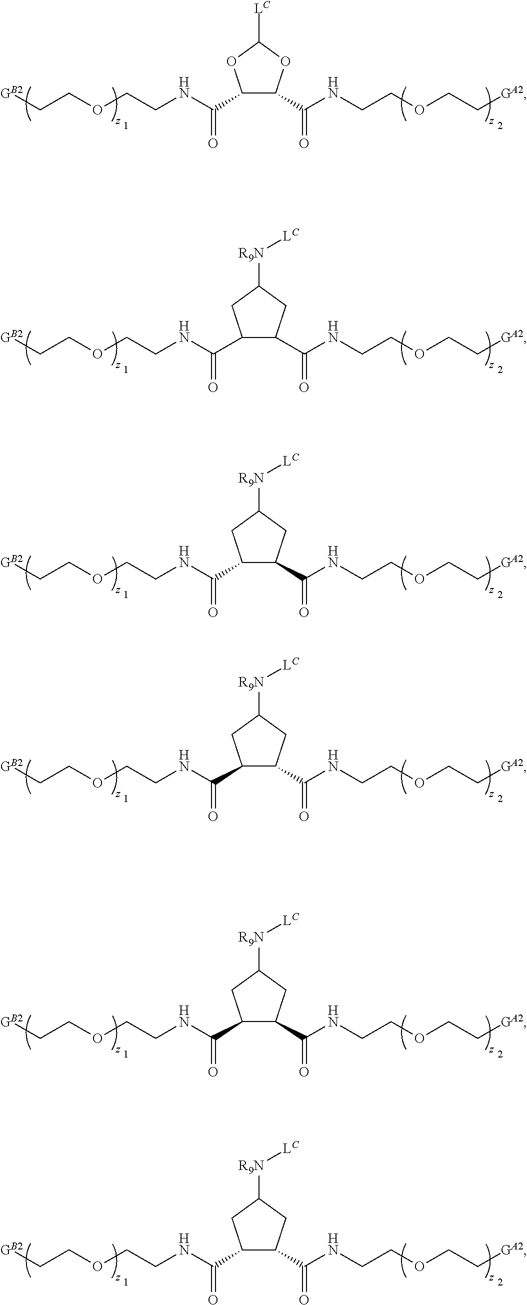 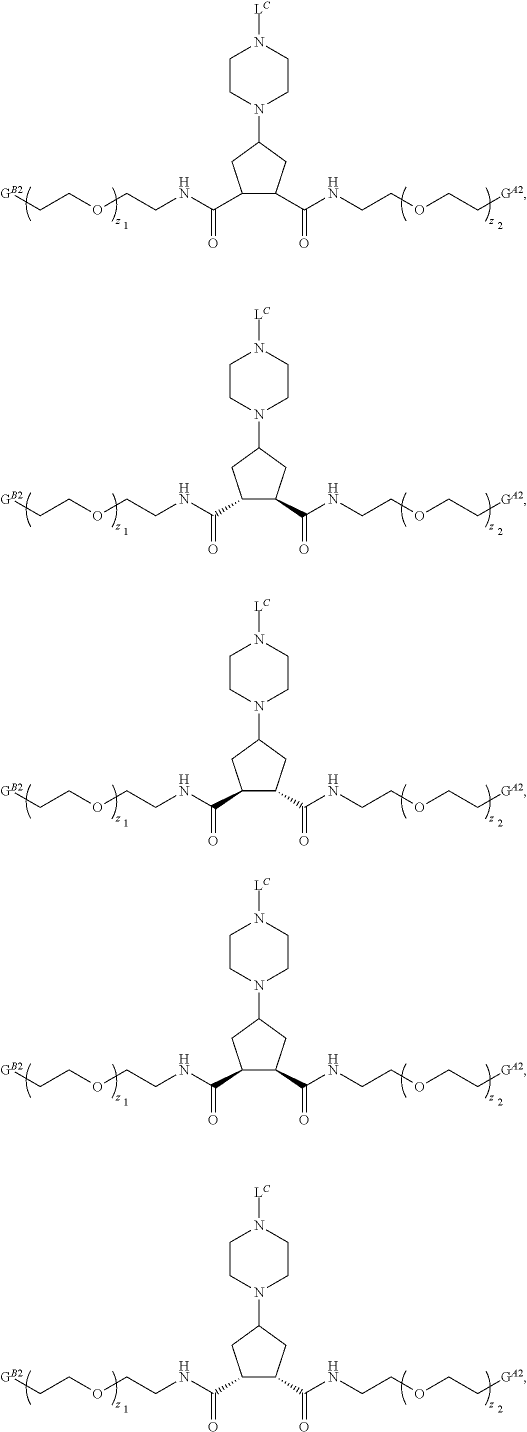 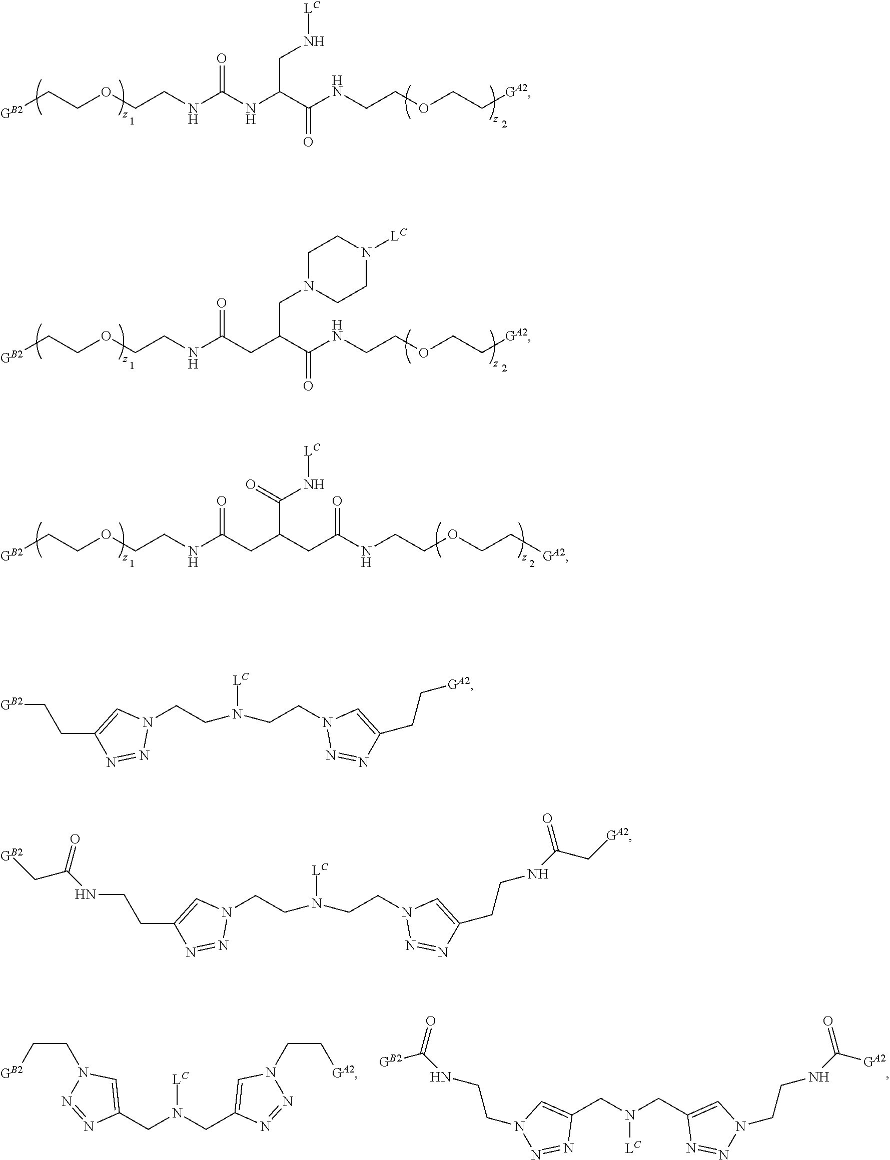 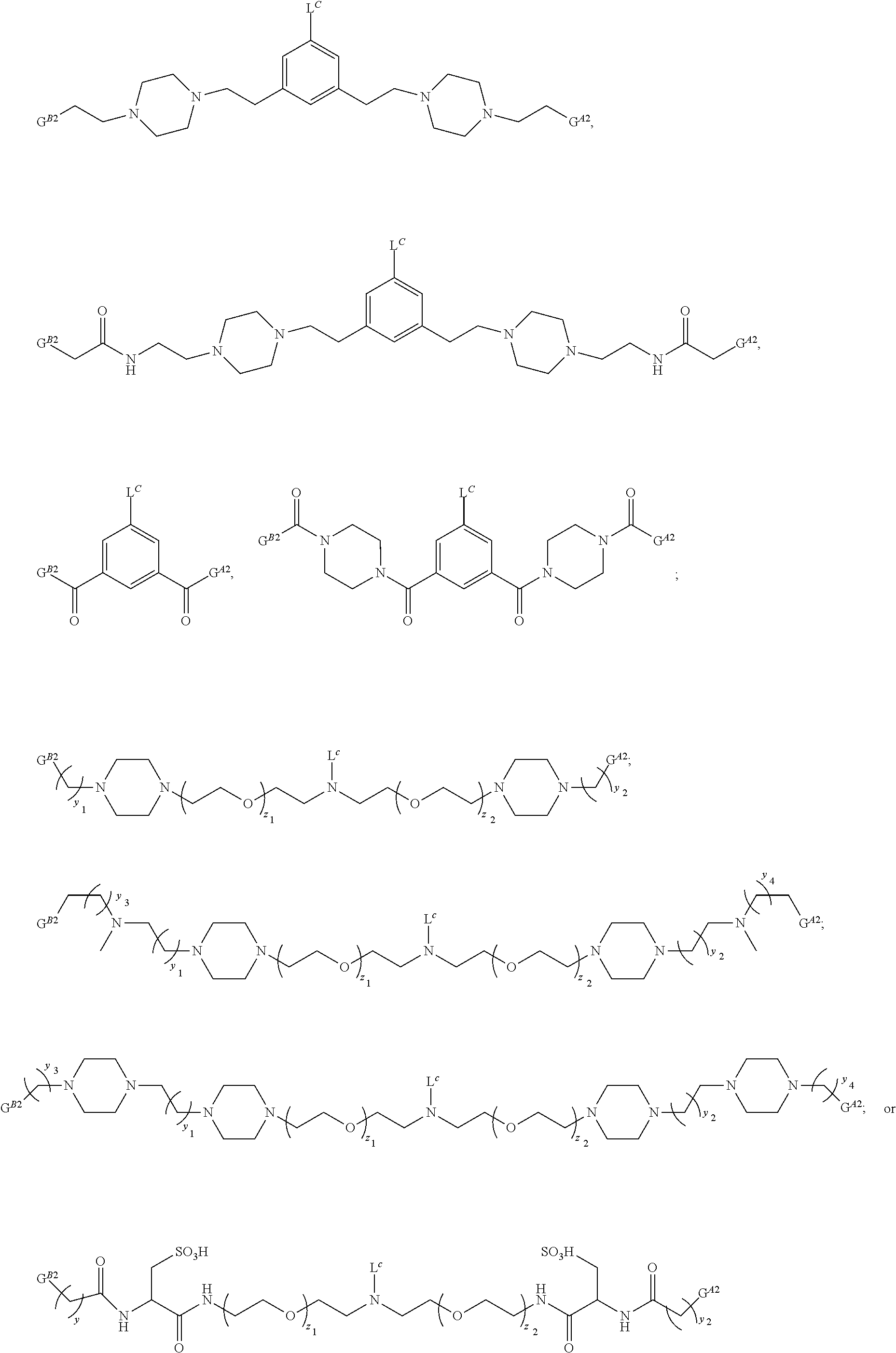
wherein z₁, z₂, y₁, y₂, y₃, and y₄ each, independently, and integer from 1 to 20; and
R₉ is selected from H, C₁-C₂₀ alkyl, C₃-C₂₀ cycloalkyl, C₂-C₂₀ heterocycloalkyl, optionally substituted C₅-C₁₅ aryl, and C₃-C₁₅ heteroaryl.

54. A conjugate described by formula (M-I): 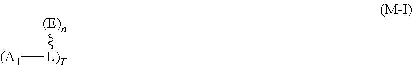
wherein each A₁ is independently selected from any one of formulas (A-I)-(A-III);
each E independently comprises an Fc domain monomer, an albumin protein, an albumin protein-binding peptide, or an Fc-binding peptide;
n is 1 or 2;
T is an integer from 1 to 20; and
L is a linker covalently attached to each of E and A₁,
or a pharmaceutically acceptable salt thereof.

55. The conjugate of claim 54, wherein the conjugate is described by formula (M-II): 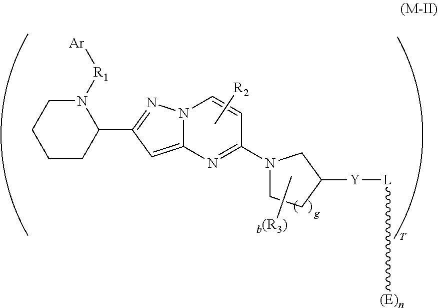
or a pharmaceutically acceptable salt thereof.

56. The conjugate of claim 55, wherein the conjugate is described by formula (M-II-1): 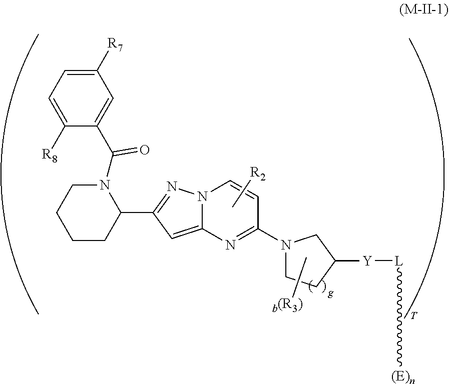
wherein R₇ and R₈ are each independently selected from OH, halogen, nitrile, nitro, optionally substituted amine, optionally substituted imine, optionally substituted C₁-C₂₀ alkamino, optionally substituted sulfhydryl, optionally substituted carboxyl, optionally substituted cyano, optionally substituted C₁-C₂₀ alkyl, optionally substituted C₃-C₂₀ cycloalkyl, optionally substituted C₂-C₂₀ alkenyl, optionally substituted C₃-C₂₀ cycloalkenyl, optionally substituted C₂-C₂₀ alkynyl, optionally substituted C₅-C₂₀ aryl, optionally substituted C₂-C₁₅ heteroaryl, and optionally substituted C₁-C₂₀ alkoxy;
or a pharmaceutically acceptable salt thereof.

57. The conjugate of claim 56, wherein the conjugate is described by formula (M-II-2): 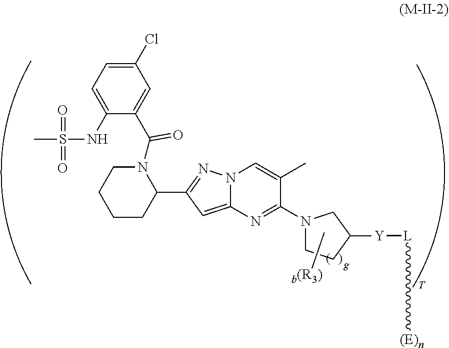
or a pharmaceutically acceptable salt thereof.

58. The conjugate of claim 57, wherein the conjugate is described by formula (M-II-3) 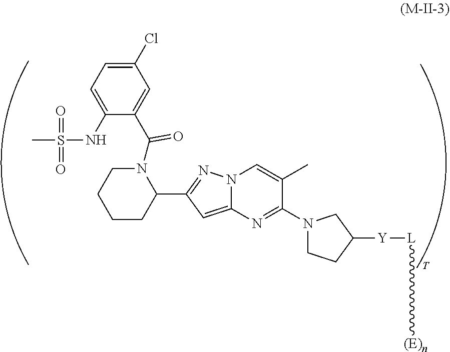
or a pharmaceutically acceptable salt thereof.

59. The conjugate of claim 58, wherein the conjugate is described by formula (M-II-4) 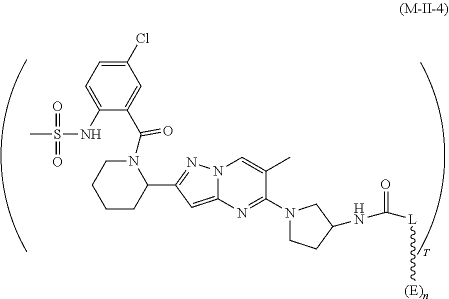
or a pharmaceutically acceptable salt thereof.

60. The conjugate of claim 59, wherein the conjugate is described by the formula (M-II-5): 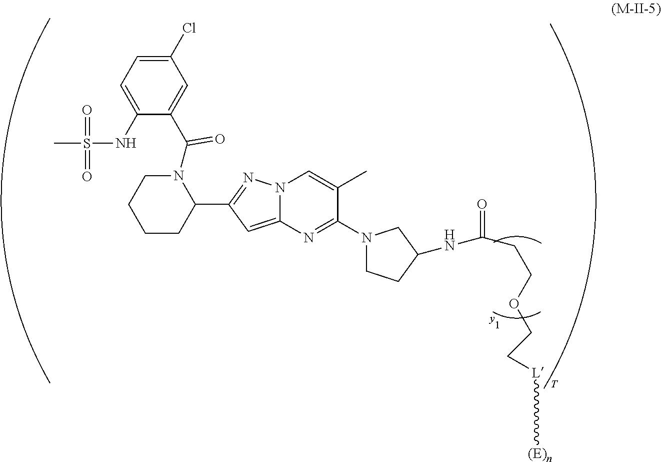
wherein L′ is the remainder of L, and
y₁ is an integer from 1-20,
or a pharmaceutically acceptable salt thereof.

61. The conjugate of claim 58, wherein the conjugate is described by formula (M-II-6): 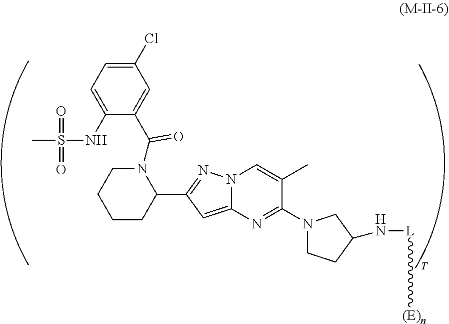
or a pharmaceutically acceptable salt thereof.

62. The conjugate of claim 61, wherein the conjugate is described by the formula (M-II-7): 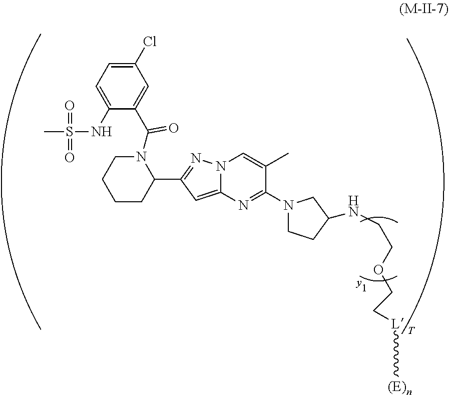
wherein L′ is the remainder of L, and
y₁ is an integer from 1-20,
or a pharmaceutically acceptable salt thereof.

63. The conjugate of claim 58, wherein the conjugate is described by formula (M-II-8): 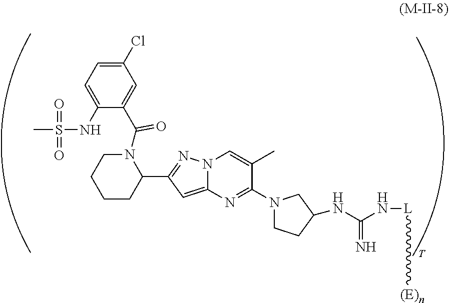
or a pharmaceutically acceptable salt thereof.

64. The conjugate of claim 63, wherein the conjugate is described by formula (M-II-9): 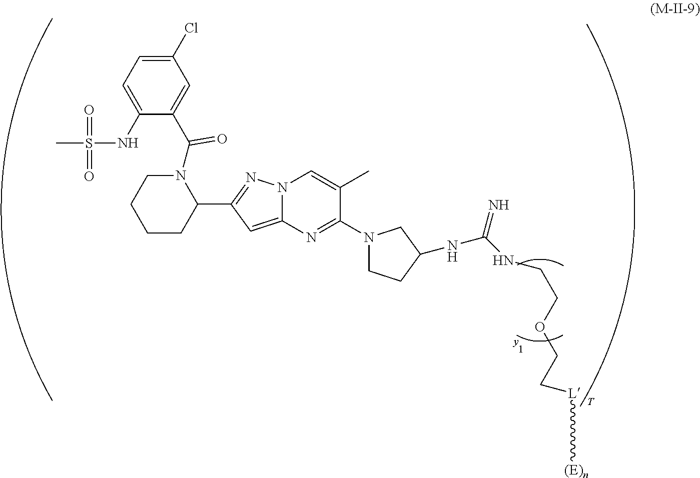
wherein L′ is the remainder of L, and
y₁ is an integer from 1-20,
or a pharmaceutically acceptable salt thereof.

65. The conjugate of claim 57, wherein the conjugate is described by the formula (M-II-10): 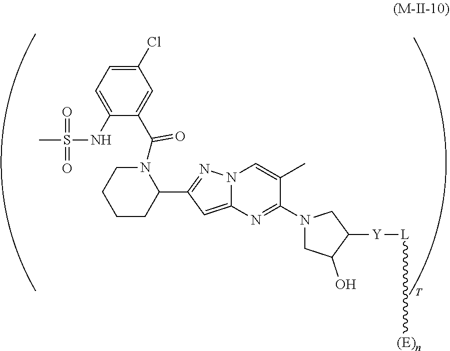
or a pharmaceutical acceptable salt thereof.

66. The conjugate of claim 65, wherein the conjugate is described by formula (M-II-11): 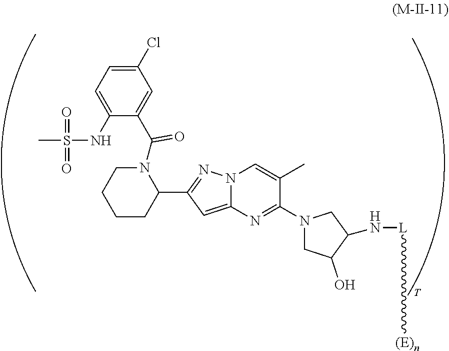
or a pharmaceutically acceptable salt thereof.

67. The conjugate of claim 66, wherein the conjugate is described by the formula (M-II-12): 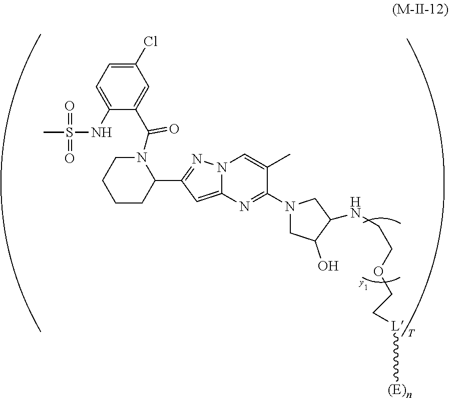
wherein L′ is the remainder of L, and
y₁ is an integer from 1-20,
or a pharmaceutically acceptable salt thereof.

68. The conjugate of claim 55, wherein the conjugate is described by formula (M-II-13): 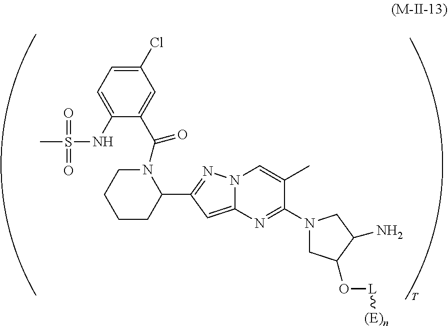
or a pharmaceutically acceptable salt thereof.

69. The conjugate of claim 68, wherein the conjugate is described by formula (M-II-14): 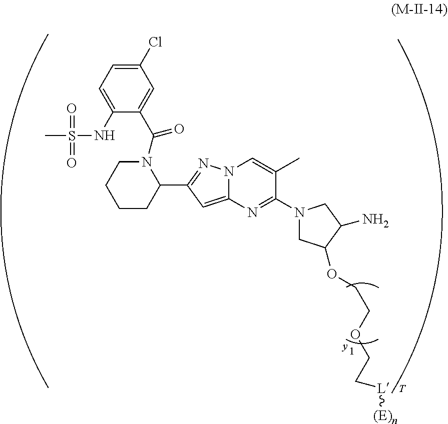
wherein L′ is the remainder of L, and
y₁ is an integer from 1-20,
or a pharmaceutically acceptable salt thereof.

70. The conjugate of claim 57, wherein the conjugate is described by formula (M-II-15): 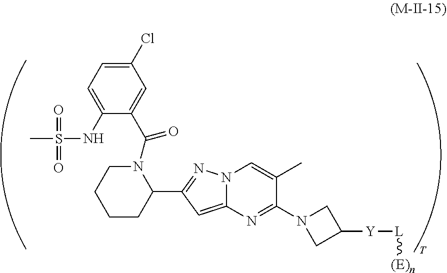
or a pharmaceutically acceptable salt thereof.

71. The conjugate of claim 70, wherein the conjugate is described by formula (M-II-16): 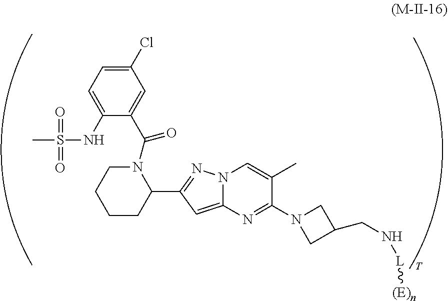
or a pharmaceutically acceptable salt thereof.

72. The conjugate of claim 71, wherein the conjugate is described by formula (M-II-17): 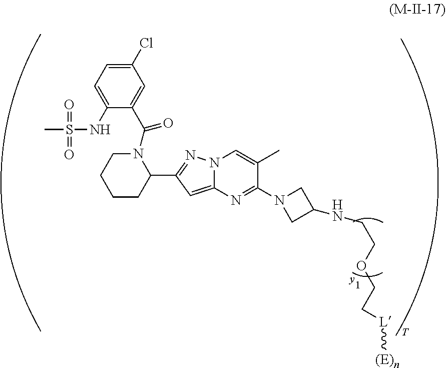
wherein L′ is the remainder of L, and
y₁ are each independently an integer from 1-20,
or a pharmaceutically acceptable salt thereof.

73. The conjugate of claim 54, wherein the conjugate is described by formula (M-III): 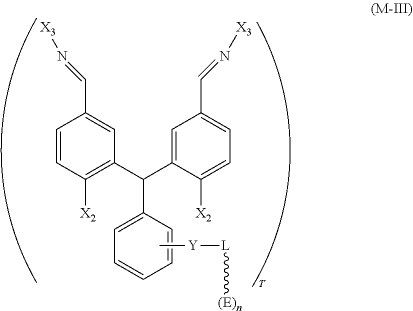
or a pharmaceutically acceptable salt thereof.

74. The conjugate of claim 73, wherein the conjugate is described by formula (M-III-1): 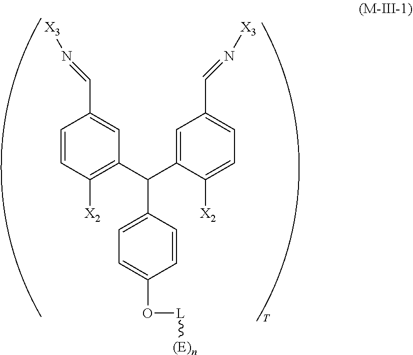
or a pharmaceutically acceptable salt thereof.

75. The conjugate of claim 74, wherein the conjugate is described by formula (M-III-2): 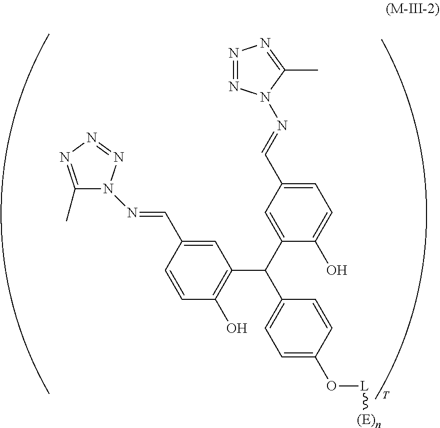
or a pharmaceutically acceptable salt thereof.

76. The conjugate of claim 75, wherein the conjugate is described by formula (M-III-3): 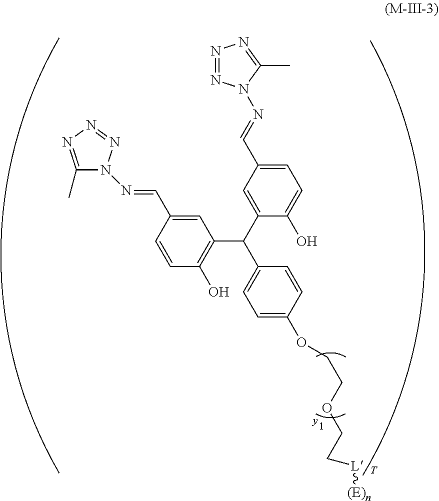
wherein L′ is the remainder of L, and
y₁ are each independently an integer from 1-20,
or a pharmaceutically acceptable salt thereof.

77. The conjugate of claim 54, wherein the conjugate is described by formula (M-IV): 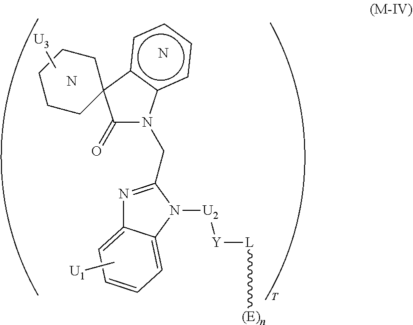
wherein U₂ is an optionally substituted C₁-C₆ alkyl,
or a pharmaceutically acceptable salt thereof.

78. The conjugate of claim 77, wherein the conjugate is described by formula (M-IV-1): 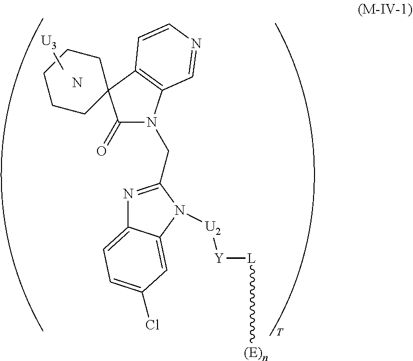
or a pharmaceutically acceptable salt thereof.

79. The conjugate of claim 78, wherein the conjugate is described by formula (M-IV-2): 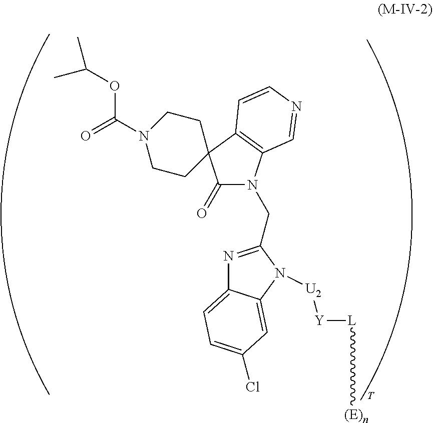
or a pharmaceutically acceptable salt thereof.

80. The conjugate of claim 79, wherein the conjugate is described by formula (M-IV-3): 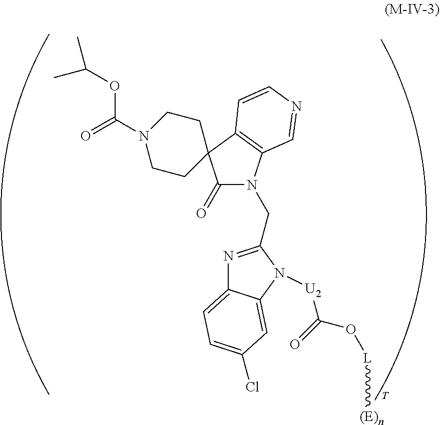
or a pharmaceutically acceptable salt thereof.

81. The conjugate of claim 80, wherein the conjugate is described by formula (M-IV-4): 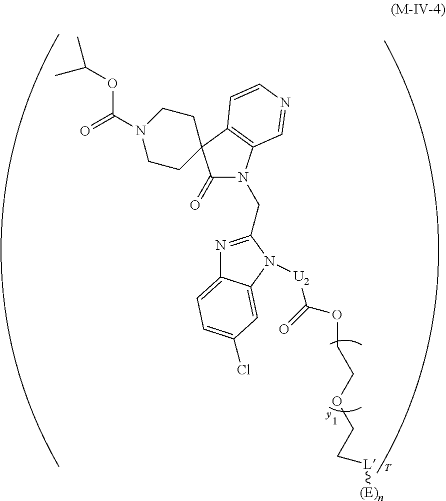
wherein L′ is the remainder of L, and
y₁ is an integer from 1-20,
or a pharmaceutically acceptable salt thereof.

82. The conjugate of claim 79, wherein the conjugate is described by formula (M-IV-5): 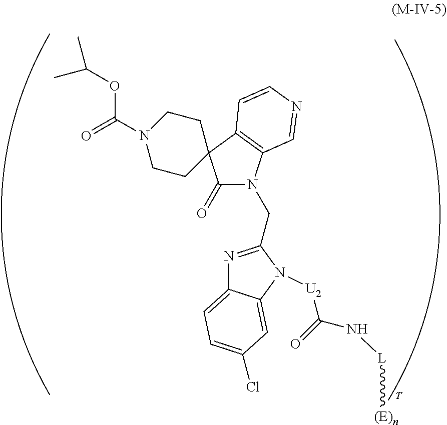
or a pharmaceutically acceptable salt thereof.

83. The conjugate of claim 82, wherein the conjugate is described by formula (M-IV-6): 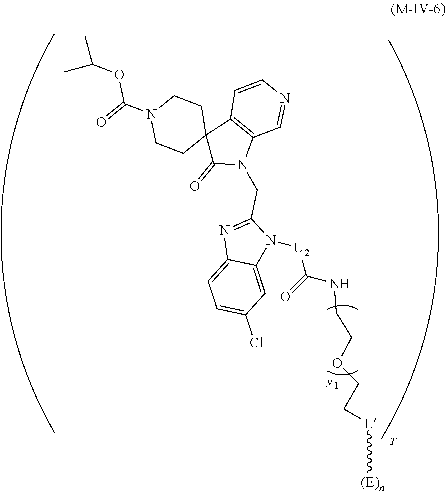
wherein L′ is the remainder of L, and
y₁ is an integer from 1-20,
or a pharmaceutically acceptable salt thereof.

84. The conjugate of claim 79, wherein the conjugate is described by formula (M-IV-7): 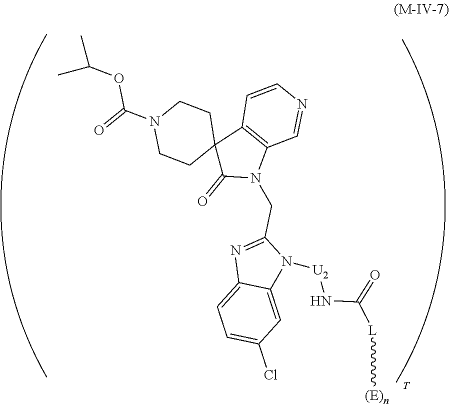
or a pharmaceutically acceptable salt thereof.

85. The conjugate of claim 84, wherein the conjugate is described by formula (M-IV-8): 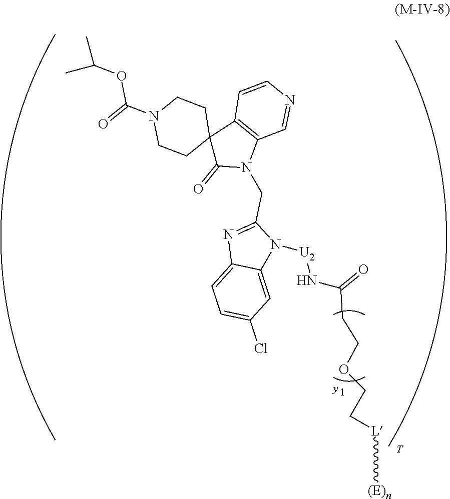
wherein L′ is the remainder of L, and
y₁ and y₂ are each independently an integer from 1-20,
or a pharmaceutically acceptable salt thereof.

86. The conjugate of claim 78, wherein the conjugate is described by formula (M-IV-9): 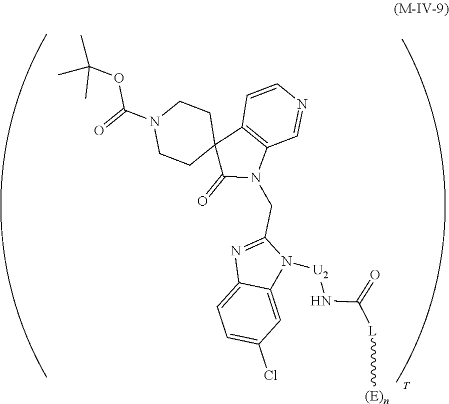
or a pharmaceutically acceptable salt thereof.

87. The conjugate of claim 86, wherein the conjugate is described by formula (M-IV-10): 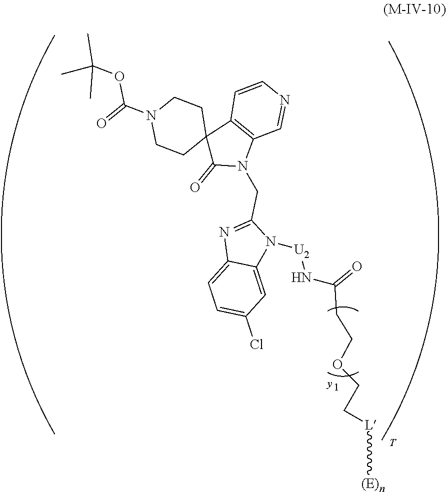
wherein L′ is the remainder of L, and
y₁ and y₂ are each independently an integer from 1-20,
or a pharmaceutically acceptable salt thereof.

88. The conjugate of claim 79, wherein the conjugate is described by formula (M-IV-11): 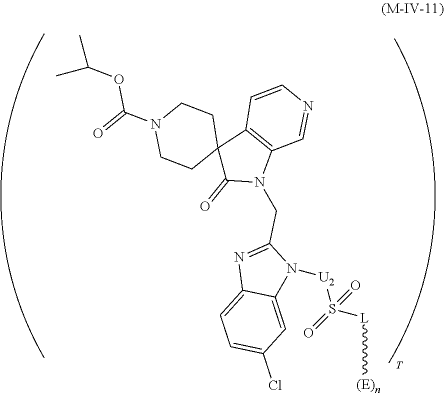
or a pharmaceutically acceptable salt thereof.

89. The conjugate of claim 88, wherein the conjugate is described by formula (M-IV-12): 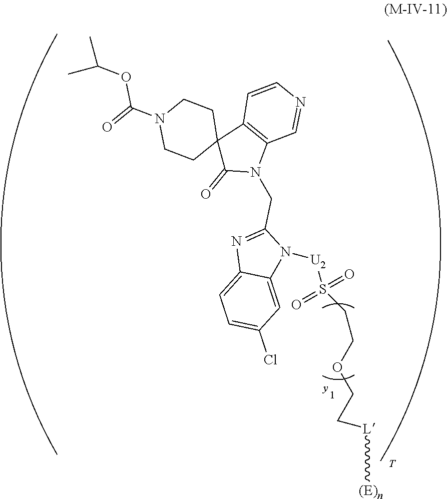
wherein L′ is the remainder of L, and
y₁ and y₂ are each independently an integer from 1-20,
or a pharmaceutically acceptable salt thereof.

90. The conjugate of claim 78, wherein the conjugate is described by formula (M-IV-13): 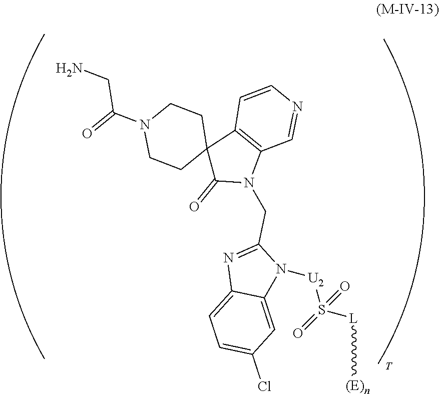
or a pharmaceutically acceptable salt thereof.

91. The conjugate of claim 90, wherein the conjugate is described by formula (M-IV-14): 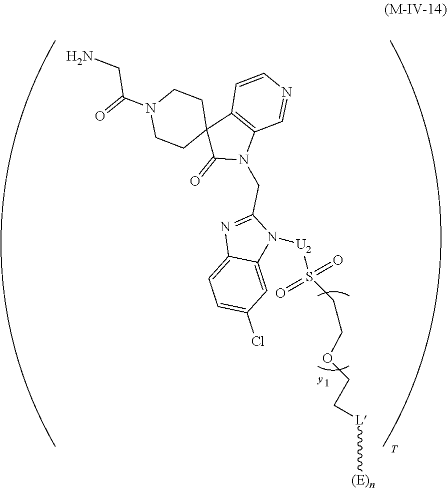
wherein L′ is the remainder of L, and
y₁ and y₂ are each independently an integer from 1-20,
or a pharmaceutically acceptable salt thereof.

92. The conjugate of claim 78, wherein the conjugate is described by formula (M-IV-15): 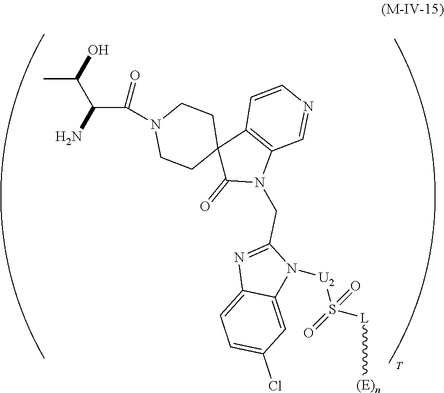
or a pharmaceutically acceptable salt thereof.

93. The conjugate of claim 92, wherein the conjugate is described by formula (M-IV-16): 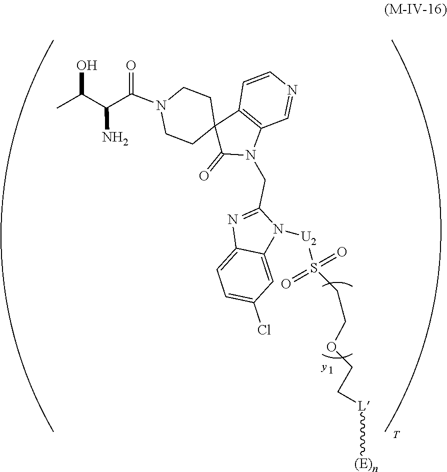
wherein L′ is the remainder of L, and
y₁ and y₂ are each independently an integer from 1-20,
or a pharmaceutically acceptable salt thereof.

94. The conjugate of claim 79, wherein the conjugate is described by formula (M-IV-17): 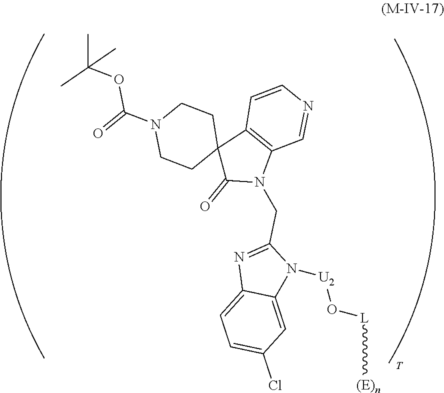
or a pharmaceutically acceptable salt thereof.

95. The conjugate of claim 94, wherein the conjugate is described by formula (M-IV-18): 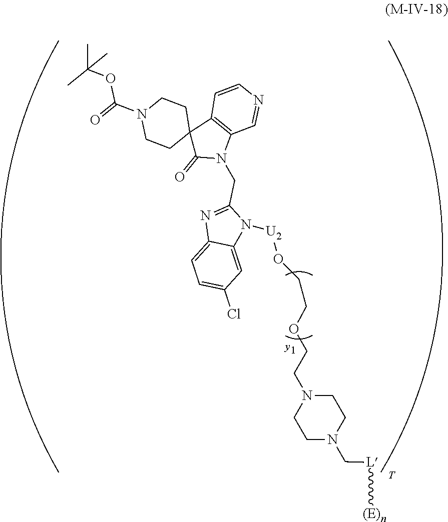
wherein L′ is the remainder of L, and
y₁ and y₂ are each independently an integer from 1-20,
or a pharmaceutically acceptable salt thereof.

96. The conjugate of any one of claims 78-95, wherein U₂ is C₂-C₆ alkyl.

97. The conjugate of any one of claims 54-96, wherein L or L′ comprises one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino, wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

98. The conjugate of claim 97, wherein the backbone of L or L′ consists of one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino, wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

99. The conjugate of claim 97 or 98, wherein L or L′ is oxo substituted.

100. The conjugate of any one of claims 54-99, wherein the backbone of L or L′ comprises no more than 250 atoms.

101. The conjugate of any one of claims 54-100, wherein L or L′ is capable of forming an amide, a carbamate, a sulfonyl, or a urea linkage.

102. The conjugate of any one of claims 54-96, wherein L or L′ is a bond.

103. The conjugate of any one of claims 54-96, wherein L or L′ is an atom.

104. The conjugate of any one of claims 54-103, wherein each L is described by formula (M-L-I):
J¹-(Q¹)_{g}-(T¹)ₕ-(Q²)ᵢ-(T²)ⱼ-(Q³)ₖ-(T³)ₗ-(Q⁴)ₘ-(T⁴)ₙ-(Q⁵)ₒ-J²
wherein J¹ is a bond attached A₁;
J² is a bond attached to E;
each of Q¹, Q², Q³, Q⁴, and Q⁵ is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene;
each of T¹, T², T³, T⁴ is, independently, O, S, NRⁱ, P, carbonyl, thiocarbonyl, sulfonyl, phosphate, phosphoryl, or imino;
Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and
each of g, h, i, j, k, l, m, n, and o is, independently, 0 or 1.

105. The conjugate of any one of claim 1-104, wherein the squiggly line connected to E indicates that the L of each A₁-L or each A₁-L-A₂ is covalently attached to a nitrogen atom of a solvent-exposed lysine of E.

106. The conjugate of any one of claim 1-104, wherein the squiggly line connected to E indicates that the L of each A₁-L or each A₁-L-A₂ is covalently attached to the sulfur atom of a solvent-exposed cysteine of E.

107. The conjugate of any one of claims 1-106, wherein each E is an Fc domain monomer.

108. The conjugate of claim 107, wherein n is 2, and each E dimerizes to form an Fc domain.

109. The conjugate of claim 2, wherein n is 2, each E is an Fc domain monomer, each E dimerizes to form an Fc domain, and the conjugate is described by formula (D-I-1): 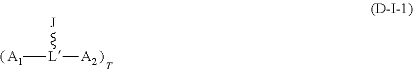
wherein J is an Fc domain; and
T is an integer from 1 to 20,
or a pharmaceutically acceptable salt thereof.

110. The conjugate of claim 54, wherein n is 2, each E is an Fc domain monomer, each E dimerizes to form an Fc domain, and the conjugate is described by formula (M-I-1): 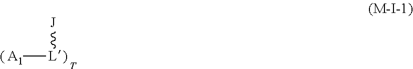
wherein J is an Fc domain; and
T is an integer from 1 to 20,
or a pharmaceutically acceptable salt thereof.

111. The conjugate of any one of claims 1-110, wherein each E independently has the sequence of any one of SEQ ID NOs: 1-95.

112. The conjugate of any one of claims 1-111, wherein T is 1, 2, 3, 4, 5, 6, 7, 8, 9, or 10.

113. A population of conjugates of any one of claims 1-111, wherein the average value of T is 1 to 10.

114. A population of conjugates of claim 113, wherein the average value of T is 1 to 5.

115. A pharmaceutical composition comprising a conjugate of any of claims 1-112, a population of conjugates of any one of claim 113 or 114, or a pharmaceutically acceptable salt thereof, and a pharmaceutically acceptable excipient.

116. A method for the treatment of a subject having a viral infection or presumed to have a viral infection, the method comprising administering to the subject an effective amount of a conjugate of any of claims 1-112, a population of conjugates of any one of claim 113 or 114, or a composition of claim 115.

117. A method for the prophylactic treatment of a viral infection in a subject in need thereof, the method comprising administering to the subject an effective amount of a conjugate of any of claims 1-112, a population of conjugates of any one of claim 113 or 114, or composition of claim 115.

118. The method of claim 116 or 117, wherein the viral infection is caused by respiratory syncytial virus.

119. The method of claim 118, wherein the RSV is RSV A or RSV B.

120. The method of any one of claims 116-119, wherein the subject is immunocompromised.

121. The method of any one of claims 116-120, wherein the subject has been diagnosed with humoral immune deficiency, T cell deficiency, neutropenia, asplenia, or complement deficiency.

122. The method of any one of claims 116-121, wherein the subject is being treated or is about to be treated with an immunosuppressive therapy.

123. The method of any one of claims 116-122, wherein said subject has been diagnosed with a disease which causes immunosuppression.

124. The method of claim 123, wherein the disease is cancer or acquired immunodeficiency syndrome.

125. The method of claim 124, wherein the cancer is leukemia, lymphoma, or multiple myeloma.

126. The method of any one of claims 116-125, wherein the subject has undergone or is about to undergo hematopoietic stem cell transplantation.

127. The method of any one of claims 116-125, wherein the subject has undergone or is about to undergo an organ transplant.

128. The method of any one of claims 116-127, wherein the subject is less than 60 months old.

129. The method of any one of claims 116-128, wherein the subject is less than 24 months old.

130. The method of claim 116-129, wherein the subject is a premature infant.

131. The method of any one of claims 116-130, wherein the conjugate of composition is administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravascularly, mucosally, intrapericardially, intraumbilically, intraocularally, orally, locally, by inhalation, by injection, or by infusion.

132. The method of any one of claims 116-131, wherein the subject is treated with a second therapeutic agent.

133. The method of claim 132, wherein the second therapeutic agent is an antiviral agent.

134. The method of claim 133, wherein the antiviral agent is selected from presatovir, lumcitabine, and ribavirin.

135. The method of claim 132, wherein the second therapeutic agent is an antiviral vaccine.

136. The method of claim 135, wherein the antiviral vaccine elicits an immune response in the subject against respiratory syncytial virus.
